(19) United States
(12) Patent Application Publication
(10) Pub. No.: US 2026/0325656 A1
(43) Pub. Date: Oct. 22, 2026

(54) NOVEL CO-DRUG, CO-ADMINISTRATION, AND SEQUENTIAL ADMINISTRATION OF SELECTIVE RBP4 ANTAGONIST AND C20-D3-RETINOL FOR ELIMINATION OF MECHANISM-BASED OCULAR ADVERSE EFFECTS IN TREATING MACULAR DEGENERATION, NON-ALCOHOLIC FATTY LIVER DISEASE (NAFLD), AND GOUTY ARTHRITIS (GOUT)

(71) Applicants: The Trustees of Columbia University in the City of New York, New York, NY (US); Rensselaer Polytechnic Institute, Troy, NY (US)

(72) Inventors: Konstantin Petrukhin, New Windsor, NY (US); Christopher L. Cioffi, Troy, NY (US)

(73) Assignees: The Trustees of Columbia University in the City of New York, New York, NY (US); Rensselaer Polytechnic Institute, Troy, NY (US)

(21) Appl. No.: 19/427,296

(22) Filed: Dec. 19, 2025

Related U.S. Application Data
(63) Continuation of application No. PCT/US2024/034834, filed on Jun. 20, 2024.
(60) Provisional application No. 63/509,182, filed on Jun. 20, 2023.

(51) Int. Cl. A61K 47/55; A61K 47/54; A61P 27/02

(52) U.S. Cl. CPC A61K 47/551; A61K 47/545; A61P 27/02

(57) ABSTRACT

New therapies for macular degeneration, non-alcoholic fatty liver disease (NAFLD), and gouty arthritis (gout) are provided based on a co-drug that represents a conjugate of two distinct chemical entities as well as co-administration and sequential administration of the two chemical entities. The first component (“selective RBP4 antagonist”) is a chemical entity that engages RBP4 (retinol binding protein 4) of the RBP4-TTR (transthyretin) complex, which is involved in delivery of retinol to the retina. This component reduces traffic of retinol from circulation to the retina. The second component (“C20-D3-visual-chromophore-producing compound”) is a C20-D3-modified retinoid or carotenoid that upon metabolism in a mammal can eventually produce a C20-D3 visual chromophore that represents C20-D3-9-cis-retinaldehyde or C20-D3-11-cis-retinaldehyde in the retina. Deuteration at the C20 position reduces the formation of lipofuscin bisretinoid while other functions (such as providing a precursor for in vivo synthesis of the visual chromophore, 11-cis-retinaldehyde) are not reduced.

This application is a continuation of PCT International Application No. PCT/US2024/034834, filed Jun. 20, 2025, claiming the benefit of U.S. Provisional Application No. 63/509,182, filed Jun. 20, 2023, the contents of each of which are hereby incorporated by reference into the subject application.

This invention was made with government support under grant number EY028549 awarded by the National Institutes of Health. The government has certain rights in the invention.

Throughout the present disclosure, certain publications are referenced in parentheses. Full citations for these publications may be found immediately preceding the claims. The disclosures of these publications in their entireties are hereby incorporated by reference into the present disclosure in order to describe more fully the state of the art to which the present disclosure relates.

### BACKGROUND OF THE INVENTION

### A. Macular Degeneration

Macular degenerations affect millions in the United States alone, with the loss of central vision greatly affecting living conditions. Many forms of the disease exist, with the most common being age-related macular degeneration (AMD). Although some treatments exist, particularly for the subset known as wet AMD, treatment is still limited for dry AMD to supportive care to delay the onset of the disease where the supportive care relates to the use of AREDS (Age-Related Eye Disease Study) formula of vitamins and nutrients. While the FDA (Food and Drug Administration) recently approved intravitreal pegcetacoplan (SYFOVRE™; Apellis Pharmaceuticals, Inc.) for treating geographic atrophy (GA) secondary to dry AMD, the treatment is based on a pegylated peptide that is a complement inhibitor. Specifically, pegcetacoplan binds to complement protein C3 and its activation fragment C3b with high affinity, thereby regulating the cleavage of C3 and the generation of downstream effectors of complement activation. The treatment is also associated with inconvenient intraocular delivery, and its efficacy is not optimal. Oral therapies, such as co-drug therapies, will have a significant advantage over pegcetacoplan in terms of patient convenience. They further have a mechanism that is independent of direct modulation of the complement cascade in the retina. An attractive target for treatment of the disease is the downregulation of serum retinol, but current approaches cause adverse events in patients, limiting their usage.

Photoreceptor loss in dry AMD is secondary to abnormalities in the Retinal Pigment Epithelium (RPE), a cellular layer that provides critical metabolic support to rods and cones. Age-dependent accumulation of lipofuscin in the RPE is associated with age-related increased incidences of dry AMD and may be one of several pathogenic factors contributing to disease onset and progression. Bisretinoids, which are byproducts of the visual cycle stemming from retinaldehyde dimerization, mediate lipofuscin toxicity and exert a variety of deleterious effects on RPE cells (Bergmann, M. et al. 2004; Sparrow, J. R. et al. 2003; Dorey, C. K. et al. 1989; Sparrow, J. R. & Boulton, M. 2005). Among them is the dysregulation of the complement system (Charbel, I. P. et al. 2015; Radu, R. A. et al. 2011; Radu, R. A. et al. 2014; Brandstetter, C. et al. 2015A) and inflammasome activation (Brandstetter, C. et al. 2015A; Brandstetter, C. et al. 2015B), the hallmarks of dry AMD pathology (Geerlings, M. J. et al. 2017).

While the accumulation of lipofuscin is one of several contributing factors underlying dry AMD pathogenesis, enhanced bisretinoid biosynthesis represents the sole causative factor underlying Stargardt disease (STGD1), a genetic form of macular degeneration (Birnbach, C. D. et al. 1994; De Laey, J. J. & Verougstraete, C. 1995; Delori, F. C. 1995; Eagle, R. C. et al. 1980). The non-enzymatic biosynthesis of cytotoxic bisretinoids involves two condensations of all-trans-retinaldehyde (Sparrow, J. R. et al. 2003) and/or 11-cis-retinaldehyde with phosphatidylethanolamine (Boyer, N. P. et al. 2012; Tang, P. H. et al. 2012). Retinaldehyde synthesis and bisretinoid production are fueled by the influx of serum retinol to the retina. Thus, the pharmacological down-regulation of serum retinol may limit retinal bisretinoid and retinaldehyde production, and represents a highly attractive target area for dry AMD and STGD1 treatment (Kennedy, C. J. et al. 1995; Radu, R. A. et al. 2005; Radu, R. A. et al. 2003; Maeda, A. et al. 2006; Palczewski, K. 2010; Cioffi, C. L. et al. 2014; Dobri, N. et al. 2013). Serum retinol is delivered to the retina from circulation in a tertiary complex with Retinol-Binding Protein 4 (RBP4) and transthyretin (TTR). Without interacting with TTR, holo-RBP4 is rapidly cleared via glomerular filtration. Given that the RBP4-TTR interaction is retinol-dependent, compounds antagonizing retinol binding to RBP4 may also induce dissociation of the RBP4-TTR complex with a subsequent reduction in circulating levels of retinol and RBP4.

Selective RBP4 antagonists are highly effective in lowering serum RBP4 and reducing bisretinoid synthesis in relevant mouse models. However, human clinical use of selective RBP4 antagonists may be associated with mechanism-based ocular adverse effects (AEs), even though no ocular adverse effects were induced in mice, as previously reported (Dobri, N. et al. 2013; Racz, B. et al. 2018). Human clinical data indicates that long-term use of fenretinide (a prototypical RBP4 antagonist) in cancer patients was associated with transient and reversible ocular AEs, such as diminished dark adaptation, in a subset of patients (yearly prevalence: 5.8-6.7%) (Camerini, T. et al. 2001). Similarly, a Phase 2 fenretinide trial in patients with dry AMD reported reversible reduction in dark adaptation in ~10% of drug-treated patients (no ocular AEs with placebo) (Mata, N. L. et al. 2013). Tinlarebant (another RBP4 antagonist) safety data presented by Belite Bio indicates that a subset of patients may develop asymptomatic delayed dark adaptation (measured only instrumentally) or symptomatic xanthopsia (abnormalities in cone vision).

Age-related macular degeneration (AMD) is the leading cause of blindness in developed countries. It is estimated that 62.9 million individuals worldwide currently have the most prevalent atrophic (dry) form of AMD; 8 million of them are Americans. Due to increasing life expectancy and current demographics, this number is expected to significantly increase in the future. With the possible exception of SYFOVRE™ mentioned above, which is a complement inhibitor indicated for the treatment of geographic atrophy (GA) secondary to age-related macular degeneration (AMD), there is currently no FDA-approved treatment for the dry form of AMD, which affects 90% of AMD patients.

### B. Non-Alcoholic Fatty Liver Disease (NAFLD)

Non-alcoholic fatty liver disease (NAFLD) encompasses a spectrum of conditions associated with lipid deposition in hepatocytes of the liver. Hepatic steatosis refers to accumulation of lipids in the liver. NAFLD is characterized by hepatic steatosis due to causes other than excessive alcohol use. Clinically, hepatic steatosis is defined as a hepatic triglyceride content that exceeds 5% of total liver weight. While simple hepatic steatosis is on the least extreme side of the NAFLD spectrum, it can progress to more severe conditions of the NAFLD spectrum such as mild hepatic steatosis and non-alcoholic steatohepatitis (NASH). NASH is the extreme form of NAFLD which is characterized by lipid accumulation in the liver combined with inflammation and hepatocellular injury or fibrosis. NASH frequently leads to severe liver complications such as cirrhosis and hepatocellular carcinoma.

NAFLD is the most common form of chronic liver disease in the United States, affecting an estimated 75 to 100 million people. There is no currently approved pharmacotherapy for any form of NAFLD. Developing a drug therapy for NAFLD is of extreme importance.

### C. Gouty Arthritis (Gout)

Gouty arthritis (gout) is the most common form of inflammatory arthritis and affects more than 8 million people in the Unites States. Uric acid is a metabolic product resulting from the metabolism of purines, which are found in many foods and in human tissue. Gout is caused by excess uric acid levels in the blood, which lead to the deposition of monosodium urate crystals in tissue. These crystals are formed when concentration of uric acid in tissues and in circulation exceeds the solubility limit, leading to gout flares. Risk factors for gout include being overweight or obese, having hypertension, alcohol intake, diuretic use, a diet rich in meat and seafood, excessive consumption of fructose, and poor kidney function.

Acute flares occur when urate crystals in the joint cause acute inflammation. A flare is characterized by pain, redness, swelling, and warmth lasting days to weeks. Pain may be mild or excruciating. Most initial attacks occur in lower extremities. The typical presentation in the metatarsophalageal joint of the great toe (podagra) is the presenting joint for 50% of people with gout. Chronic gout is characterized by chronic arthritis, with soreness and aching of joints. People with gout may also get tophi or lumps of urate crystals deposited in soft tissue. Clinically inactive (intercritical) segments between gout flares occur after an acute flare has subsided. A person with gout continues to have hyperuricemia, which results in continued deposition of urate crystals in tissues and resulting damage. Intercritical segments become shorter as the disease progresses.

Uric acid is synthesized from its precursor, xanthine, by an enzyme called xanthine oxidase (XO). Accordingly, XO inhibitors (e.g., allopurinol and febuxostat) dominate the market. However, elevated levels of circulating uric acid most commonly result from under secretion of uric acid in the kidneys. Marginally effective probenecid and recently approved lesinurad are the treatments that increase the renal secretion of uric acid.

The incidence and prevalence of gout is rising. This is due to factors such as an increase in the aged population, many of whom take thiazide diuretics and prophylactic aspirin that promote hyperuricemia and lifestyle factors characterized by diets that include excessive fructose and alcohol intake, physical inactivity, and abdominal fat accumulation which favor hyperuricemia.

Significant unmet clinical need remains in the treatment of gout. Of the 8 million patients with gout, over 3 million are on urate-lowering therapy (mainly XO inhibitors). Despite this fact, 1 million patients continue to experience 3 or more flares per year, indicating the need for better urate-lowering therapy.

### SUMMARY OF THE INVENTION

The present disclosure provides two-component co-drug, co-administration, and sequential-administration strategies that combine the ability to reduce retinol while simultaneously mitigating the adverse events associated with loss of retinol. The co-drug, as a representative example, is formed from the conjugation of a RBP4-lowering compound with a deuterated retinol. The first component (“selective RBP4 antagonist”) treats the disease by limiting delivery of retinol while the second component (“C20-D3-visual-chromorphore-producing compound”) suppresses production of additional lipofuscin bisretinoid and reduces the adverse effects associated with partial reduction in production of the visual chromophore, 11-cis-retinaldehyde, in the retina. As such, the present disclosure provides means to overcome the toxicity associated with current macular degeneration treatments while still maintaining the pharmacological effects.

The first component, a “selective RBP4 antagonist,” is a chemical entity that engages the RBP4 of the RBP4-TTR complex, which is involved in delivery of retinol to the retina. The functional purpose of this component is to partially reduce traffic of retinol from circulation to the retina.

Specifically, a “selective RBP4 antagonist” is a compound having the structure:

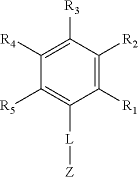

wherein L is a linking group having the structure

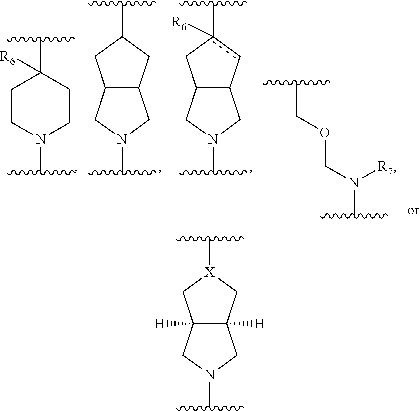

and
Z is a group having the structure

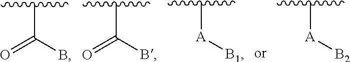

wherein
R₁, R₂, R₃, R₄, and R₅ are each independently H, halogen, CF₃, C₁-C₄ alkyl, aryl, or heteroaryl;
R₆ is H, OH, or halogen, or is absent;
ψ is absent or present, and when present, is a bond;
B is a substituted or unsubstituted heterobicycle, pyridazine, pyrazole, pyrazine, thiadiazole, or triazole, wherein
the heterobicycle is other than chloro-substituted indole; and
the pyrazole, when substituted, is substituted with other than trifluoromethyl;
   B′ is a substituted or unsubstituted phenyl, pyridine, pyrimidine, benzyl, pyrrolidine, sulfolane, oxetane, CO₂H, or (C₁-C₄ alkylenyl)-CO₂H, wherein
the substituted phenyl is substituted with other than trifluoromethyl or 3-(methyl carboxylate),
the substituted pyridine is substituted with other than trifluoromethyl,
the substituted pyrrolidine is substituted with other than hydroxamic acid, and
the substituted or unsubstituted pyrrolidine is bound to the carbonyl through a carbon-carbon bond;
   A is absent or present, and when present, is or Av;

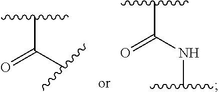

B₁ is a substituted or unsubstituted monocycle, bicycle, heteromonocycle, heterobicycle, benzyl, CO₂H, or (C₁-C₄ alkylenyl)-CO₂H,
wherein when B₁ is CO₂H, then A is present and is;

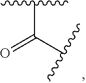 [SMILES: *C(*)=O]

R₇ is alkyl;

### X is N or CR₈,

wherein R₈ is H, OH, or halogen; and
   B₂ has the structure:

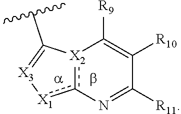

wherein
α and β are each a bond that is present or absent;
X₁ is N, NH, or NR₉₉,
wherein R₉₉ is alkyl, alkenyl, or alkynyl;

### X₂ is C or N;

### X₃ is CH or N;

R₉, R₁₀, and R₁₁ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃, wherein
X₁, X₂, and X₃ are each N, α is present, and β is absent; or
X₁ is NH, X₂ is C, X₃ is CH, α is absent, and β is present; or
X₁ is N, X₂ is N, X₃ is CH, α is present, and β is absent; or
X₁ is NH or NR₉₉, X₂ is C, X₃ is N, α is absent, and β is present,
   wherein when X₁ is NH, X₂ is C, X₃ is N, α is absent, and β is present, then one of R₉, R₁₀, and R₁₁ is other than H;
      or
      B₂ has the structure:

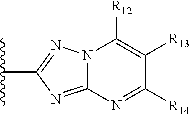

wherein
R₁₂, R₁₃, and R₁₄ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
or a pharmaceutically acceptable salt thereof.

The second component, a “C20-D3-visual-chromophore-producing compound,” is defined as any C20-D3-modified retinoid or carotenoid that upon metabolism in a mammal can eventually produce a C20-D3 visual chromophore that represents C20-D3-9-cis-retinaldehyde or C20-D3-11-cis-retinaldehyde in the retina. Examples of a C20-D3-visual-chromophore-producing compound include C20-D3-retinol, which is a deuterated form of vitamin A (retinol), C20-D3-retinaldehyde, C20-D3-retinyl esters (such as C20-D3-retinyl acetate), C20-D3-9-cis-retinol, C20-D3-9-cis-retinaldehye, C20-D3-9-cis-retinyl esters (such as C20-D3-9-cis-retinyl acetate), C20-D3-11-cis-retinol, C20-D3-11-cis-retinaldehye, C20-D3-11-cis-retinyl esters (such as C20-D3-11-cis-retinyl acetate), and C20-D3-C20′-D3-β-carotene. The deuteration at the C20 position reduces the formation of lipofuscin bisretinoid while other functions (such as providing a precursor for in vivo synthesis of the visual chromophore, 11-cis-retinaldehyde) are not reduced. Additional deuterations at positions other than C20 are possible.

When absorbed in the body of a mammal, the C20-D3-visual-chromophore-producing compound initially generates C20-D3-retinol in a form of one of three stereoisomers: C20-D3-all-trans-retinol, C20-D3-9-cis-retinol, and C20-D3-11-cis retinol. Upon delivery to the retina, deuterated all-trans-retinol undergoes isomerization by retinoid isomerohydrolase RPE65 to form 11-cis-retinol which in turn is converted to 11-cis-retinaldehyde, a visual chromophore. In contrast, cis retinoids can directly produce the visual chromophore without requiring this isomerization reaction. The visual chromophore can be either 11-cis-retinaldehyde (a natural chromophore) or 9-cis-retinaldehyde (an artificial chromophore that can bind to opsin and form rhodopsin similarly to 11-cis-retinaldehyde).

Various ratios of the first component to the second component may be used. As a co-drug, the ratio may be a 1:1 conjugate in its simplest form. The co-drug may be designed following general conjugating approaches developed for the antibody-drug conjugate (ADC) system. However, in contrast to ADC, the co-drug of the present disclosure is designed to degrade and release the first and second components in the gastrointestinal (GI) tract, or via hydrolysis in blood or lymphatic circulation, not in the target cell as is typical for ADC.

Moreover, the co-drug of the present disclosure is designed to be used orally, while ADC therapies need to be administered by non-oral routes (such as intravenously or subcutaneously).

The first component (selective RBP4 antagonist) will limit the RBP4-mediated delivery of vitamin A to the retina to inhibit bisretinoid production. Regarding the second component (C20-D3-visual-chromophore-producing compound), the fate of the released C20-D3-retinol will be as follows. It is known that 25-33% of postprandial retinoid-laden chylomicrons are taken by extrahepatic vitamin A-dependent tissues such as the retina (Vogel, S. et al. 2002). Like dietary retinoids, C20-D3-retinol will be esterified in the GI tract, packaged in chylomicrons, and delivered to the retina. Consistent chylomicron delivery of C20-D3-retinoids to the retina will provide a reliable supply of retinoids for the synthesis of visual chromophore (11-cis-retinal) and rhodopsin in visual cycle reactions (Charbel, I. P. et al. 2015), thus partially compensating for the blocking of the RBP4-mediated route and reducing related ocular AEs. At the same time, C20-D3-retinoids will not contribute to the biosynthesis of bisretinoids due to a kinetic isotope effect that slows bisretinoid synthesis (Charbel, I. P. et al. 2015; Kaufman, Y. et al. 2011; Ma, L. et al. 2011). Rhodopsin represents a protein called opsin conjugated with the visual chromophore called 11-cis-retinaldehyde; 9-cis-retinaldehyde can also serve as a visual chromophore in place of 11-cis-retinaldehyde. Apart from rhodopsin, there are three cone opsins (blue, red, and green opsins) expressed in three types of cone photoreceptors (blue, red, and green cones). The co-drug, co-administration, and sequential administration of the present disclosure provide visual chromophores to supplement all four types of functional opsins (rhodopsin, blue-opsin, green-opsin, and red-opsin).

The first and second components may also be co-administered as separate chemical entities simultaneously, contemporaneously, or concomitantly (that is, without being conjugated or bonded to each other), or they may be administered sequentially in a suitable order in which the first component is administered first or the second component is administered first. In addition, the second component (C20-D3-visual-chromophore-producing compound) may be administered in a form packaged in chylomicrons.

In the co-drug and co-administration approaches of the present disclosure, significantly improved patient compliance can be expected. The co-drug approach will also provide more predictable pharmacokinetics (PK) and pharmacodynamics (PD), and may further avoid potential adverse drug-drug interactions (DDIs) that could arise from multiple drug intakes.

### BRIEF DESCRIPTION OF THE FIGURES

FIG. 1. All-trans-retinol (vitamin A) (1), retinoic acid (2) (a vital retinoid involved in morphogenesis), 11-cis-retinal (3) (a key retinoid required for phototransduction), and the thyroid hormone thyroxine (T4) (4).

FIG. 2. Examples of previously reported RBP4 antagonists that include fenretinide (5), A1120 (6), non-retinoid [3.3.0]-octahydrocyclopenta[c]pyrrolo RBP4 antagonist 7, BPN-14136 (8), [3.3.0]-octahydrocyclopenta[c]pyrrolo RBP4 antagonist 9, and [1,2,4]triazolo[4,3-a]pyridine RBP4 antagonist BPN-14634 (10).

FIG. 3. Syntheses of C20-D3-retinol (9) and C20-D3-retinyl acetate (10) (Bergen, H. R. et al. 1988).

FIG. 4. Synthesis of C20-D3-9-cis-retinol based on existing routes for synthesizing 9-cis-retinol (Korean Patent No. 10-2271364 B1) and for incorporating deuterium at the C20 position (Bergen, H. R. et al. 1988).

FIG. 5. Synthesis of C20-D3-11-cis-retinol based on existing routes for synthesizing 11-cis-retinol (Borhan, B. et al. 1999) and for incorporating deuterium at the C20 position (O'Broin, C. Q. & Guiry, P. J. 2020).

FIG. 6. Synthesis of C20-D3-C20′-D3-3-carotene based on existing routes for synthesizing C20-D3-all-trans-retinol (Bergen, H. R. et al. 1988) and β-carotene (Goswami, B. C. & Barua, A. B. 2003).

FIG. 7. Medicinal chemistry co-drug strategy. (A) Design principles of a selective RBP4 antagonist with C20-D3-retinol in a single chemical entity. (B) Examples of a co-drug core that can provide different molar ratios of selective RBP4 antagonist to C20-D3-retinol. Tinlarebant is shown as the selective RBP4 antagonist. (C) Examples of co-drugs whereby C20-D3-retinol is linked to the cores via various low-pH and/or proteolytic cleavable linkers that are successfully used in ADC development. The selective RBP4 antagonists are linked via labile esters.

FIG. 8. Examples of co-drugs of tinlarebant and C20-D3-all-trans-retinol. These compounds may be synthesized based on the disclosures in WO 2015/179441 A2, WO 2003/059878 A2, WO 2023/018643 A1, and WO 2018/060481 A1. In these examples, the pyrazole nitrogen of tinlarebant provides an eventual connection point with C20-D3-all-trans-retinol. In (A), the connection point (a hemiaminal-type linker) is unstable at low pH, and the co-drug will degrade in the stomach to release tinlarebant and C20-D3-all-trans-retinol. In (B), the connection point (a carbamate linker) is labile at low pH and in the presence of esterase and/or protease (such as lipase).

FIG. 9. Structure of bisretinoids A2E and isoA2E, cytotoxic components of retinal lipofuscin.

FIG. 10. Structure of bisretinoids at RAL di-PE (all-trans-retinal dimer-phosphatidyl ethanolamine) and A2-DHP-PE, cytotoxic components of retinal lipofuscin. R₁ and R₂ refer to various fatty acid constituents.

FIG. 11. Proposed Tinlarebant-C20D3-all-trans-Retinol Co-Drugs. Pyrazole nitrogen of tinlarebant provides potential handle to connect with C20D3-all-trans-retinol. Connection point in A is unstable at low pH; should degrade in the stomach to release tinlarebant and C20D3-all-trans-retinol. Connection point (carbamate) in B is also predicted to be labile in the presences of esterases and proteases.

FIG. 12. Proposed Tinlarebant-C20D3-all-trans-Retinol Co-Drug A. Connection point in A is unstable at low pH; it should degrade in the stomach to release tinlarebant and C20D3-all-trans-retinol.

FIG. 13. Proposed Tinlarebant-C20D3-all-trans-Retinol Co-Drug A. It is expected that co-drug A would be unstable and degrade into tinlarebant and C20D3-all-trans-retinol in the low pH environment of the stomach.

FIG. 14. Proposed Tinlarebant-C20D3-all-trans-Retinol Co-Drug B. Connection point (carbamate) in B is also predicted to be labile in the presences of esterases and proteases.

FIG. 15. Proposed Tinlarebant-C20D3-all-trans-Retinol Co-Drug C.

FIG. 16. Proposed Tinlarebant-C20D3-all-trans-Retinol Co-Drug C: Fatty Acid Mimic and Pepsin/Chymotrypsin Recognition Tether.

FIG. 17. Proposed Tinlarebant-C20D3-all-trans-Retinol Co-Drug D: Silyl Ether Tether.

FIG. 18. Proposed Tinlarebant-C20D3-all-trans-Retinol Co-Drug D: Silyl Ether Tether.

FIG. 19. Triazolo nitrogen of BPN-14634 provides potential handle to connect with C20D3-all-trans-retinol. Connection point in A is unstable at low pH; should degrade in the stomach to release BPN-14634 and C20D3-all-trans-retinol. Linker in co-drug B and C is also predicted to be labile in the presences of esterases and proteases.

FIG. 20. Proposed BPN-14634-C20D3-all-trans-Retinol Co-Drug A.

FIG. 21. Proposed BPN-14634-C20D3-all-trans-Retinol Co-Drug B.

FIG. 22. Proposed BPN-14634-C20D3-all-trans-Retinol Co-Drug B. Tether based on nucleotide prodrug strategy. BPN-14634 can be liberated either via direct hydrolysis of the methylene linker or via degradation of the linker after carbamate hydrolysis (carbamate pathway).

FIG. 23. Proposed BPN-14634-C20D3-all-trans-Retinol Co-Drug C. Tether based on nucleotide prodrug strategy. BPN-14634 can be liberated either via direct hydrolysis of the methylene linker or via degradation of the linker after ester hydrolysis (similar to the carbamate pathway shown in FIG. 22).

FIG. 24. Effect of β-carotene on rhodopsin levels in ACPHS-14-treated Balb/c mice.

FIG. 25. Effect of β-carotene on scotopic ERG a-wave amplitude elicited at the 1.89 log cd*s/m² light intensity in ACPHS-14-treated Balb/c mice.

FIG. 26. Effects of C20-D3-retinyl acetate on rhodopsin levels in ACPHS-52-treated Balb/c mice. Rhodopsin was measured spectrophotometrically in retinal extracts (n=8) from dark-adapted treated and control mice. Treated mice were given 7 daily oral dose of 25 mg/kg ACPHS-52 or the same dose of ACPHS-52 along with 3.3 mg/kg C20-D3-retinyl acetate. Untreated control mice were kept on standard diet. An unpaired t-test on the ungrouped data revealed a statistically significant 1.8-fold increase in rhodopsin concentration in mice co-treated with C20-D3-retinyl acetate and ACPHS-52 in comparison to mice treated only with ACPHS-52 (P<0.05).

### DETAILED DESCRIPTION OF THE INVENTION

The present disclosure provides new therapies based on a co-drug that represents a conjugate of two distinct chemical entities, co-administration of the two chemical entities, and sequential administration of the two chemical entities.

### 1. Selective RBP4 Antagonists

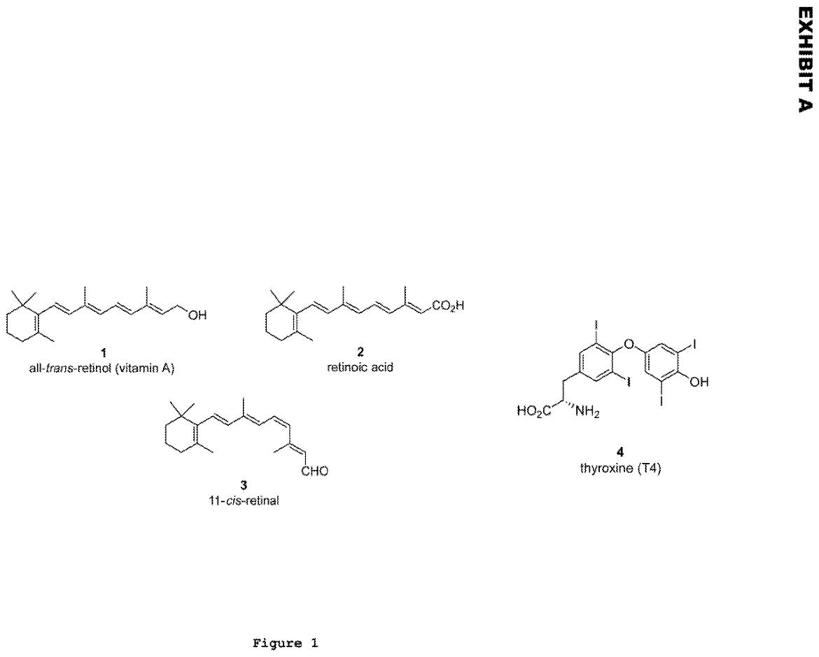

Age-related macular degeneration (AMD) is the most common cause of blindness in developed countries. Age-dependent accumulation of cytotoxic lipofuscin bisretinoids in the retina may significantly contribute to pathogenesis of the atrophic form of AMD. The essential vitamin all-trans-retinol (vitamin A, 1) (FIG. 1) serves as a precursor for the biosynthesis of retinoic acid (2) (Steinmetz, A. C. et al. 2001; Clagett-Dame, M. & DeLuca, H. F. 2002; Wolf, G. 1984), 11-cis-retinal (3) (Kiser, P. D. et al. 2014; Tsin, A. et al. 2018), and many other key retinoids involved in multiple cellular processes and numerous critical biological functions throughout the body. Bisretinoid synthesis in the eye depends on the influx of all-trans-retinol (1) from the serum to the retina. Formation of the tertiary retinol-binding protein 4 (RBP4)-transthyretin (TTR)-retinol complex in the serum is required for this influx. Reducing circulating levels of RBP4 and 1 via selective antagonists could modulate the visual cycle, reduce the rate of cytotoxic bisretinoid formation in the retinal pigment epithelium (RPE), and halt geographic atrophy in patients with atrophic (dry) age-related macular degeneration (AMD) and Stargardt disease (Radu, R. A. et al. 2005; Palczewski, K. 2010; Petrukhin, K. 2013; Petrukhin, K. 2007).

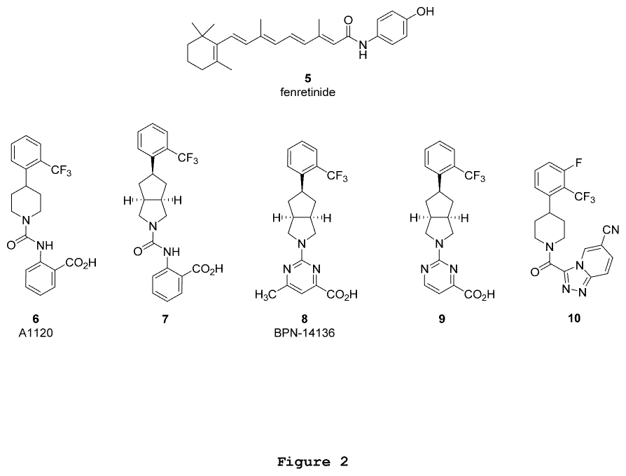

Competitive RBP4 antagonists that displace 1 prevent the formation of the holo-RBP4-TTR complex, thereby inducing reductions in circulating RBP4 and 1 levels via rapid renal clearance. A diminished influx of 1 to the RPE results in a reduction of cytotoxic bisretinoid accumulation in the retina, which is believed to underlie parts of the pathophysiology of dry AMD and Stargardt disease (Radu, R. A. et al. 2005; Dobri, N. et al. 2013; Racz, B. et al. 2018; Young, R. W. 1987; Dorey, C. K. et al. 1989; Holz, F. G. et al. 2001; Holz, F. G. et al. 1999; Holz, F. G. et al. 2007; Schmitz-Valckenberg, S. et al. 2009; Finnemann, S. C. et al. 2002; Suter, M. et al. 2000; Sparrow, J. R. et al. 2003; Sparrow, J. R. et al. 2012; Delori, F. C. 1995; Weng, J. et al. 1999; Sparrow, J. R. & Cai, B. 2001; Bergmann, M. et al. 2004; Sparrow, J. R. et al. 1999; De, S. & Sakmar, T. P. 2002; Vives-Bauza, C. et al. 2008; Zhou, J. et al. 2006; Radu, R. A. et al. 2011; Ben-Shabat, S. et al. 2002; Rozanowska, M. et al. 1995; Sparrow, J. R. et al. 2002; Dontsov, A. E. et al. 2009). The approach is supported by proof-of-concept data obtained for fenretinide (5) (FIG. 2), which was studied in the preclinical Abca4^{-/-} transgenic mouse model of enhanced retinal lipofuscinogenesis (Radu, R. A. et al. 2005) and in an extended Phase II clinical trial with dry AMD patients (Berni, R. & Formelli, F. 1992; Adams, W. R. et al. 1995; Mata, N. L. et al. 2013). The non-retinoid RBP4 antagonist A1120 (6) (Motani, A. et al. 2009) was found to lower circulating RBP4 plasma levels in rodents by >70% and reduce retinal bisretinoid accumulation in Abca4^{-/-} mice (Dobri, N. et al. 2013). Selective and orally bioavailable non-retinoid RBP4 antagonists 7 (Cioffi, C. L. et al. 2014) and BPN-14136 (8) (Cioffi, C. L. et al. 2015) displayed favorable pharmacokinetic (PK) profiles and induced dose-dependent reductions in rodent plasma RBP4 levels in both acute and chronic dosing studies. Compound 8 also robustly lowered serum RBP4 levels and exhibited excellent pharmacokinetic-pharmacodynamic (PK/PD) correlations in non-human primates upon oral administration (Racz, B. et al. 2020). Compound 8 inhibited lipofuscin bisretinoid synthesis with concomitant normalization of retinal complement system protein expression in the Abca4^{-/-} mouse model without altering visual cycle kinetics at doses inducing maximal serum RBP4 reduction (Racz, B. et al. 2018).

In addition to transporting 1 to targeted tissues, RBP4 has also been identified as an adipokine, and epidemiological evidence suggests that moderately elevated levels of the protein positively correlate with type 2 diabetes (Graham, T. E. et al. 2006; Yang, Q. et al. 2005), obesity (Aeberli, I. et al. 2007), insulin resistance (Kowalska, I. et al. 2008), cardiovascular disease (Ingelsson, E. et al. 2009; Qi, Q. et al. 2007; Norseen, J. et al. 2012), and hepatic steatosis (Lee, S. A. et al. 2016). Thus, the pharmacological reduction of circulating RBP4 serum levels may also hold promise for the treatment of a myriad of metabolic diseases. It has recently been reported that RBP4 antagonist 10 (BPN-14634) significantly lowered serum RBP4 levels in rodents (>80%), reduced the concentration of circulating RBP4 produced in the adipose tissue, and demonstrated efficacy in the transgenic adi-hRBP4 murine model of hepatic steatosis, suggesting that it may have therapeutic utility for the treatment of non-alcoholic fatty liver disease (NAFLD) (Cioffi, C. L. et al. 2019).

International Patent Application No. PCT/US2019/044754, published as WO 2020/028723 A1 and hereby incorporated by reference in its entirety, discloses a class of non-retinoid RBP4 antagonists (“selective RBP4 antagonists” in the present disclosure) that are shown to bind RBP4 in vitro and/or to antagonize RBP4-TTR interaction in vitro at biologically significant concentrations, and hence be effective to treat not only macular degeneration but also non-alcoholic fatty liver disease (NAFLD) and gouty arthritis (gout) by normalizing the concentrations of triglycerides and free fatty acids in the liver. Examples of selective RBP4 antagonists include

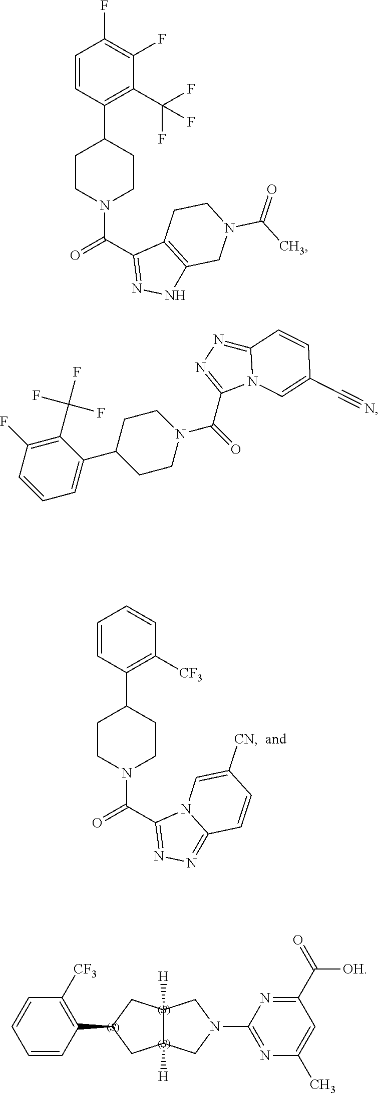

In particular, the RBP4 antagonist named tinlarebant (also known by the designated codes of “BPN-14697” and “LBS-008”) and shown below

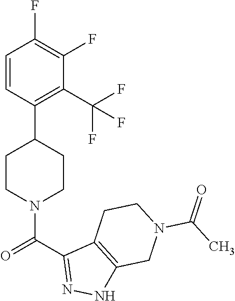 [SMILES: CC(=O)N1CCc2c(C(=O)N3CCC(c4ccc(F)c(F)c4C(F)(F)F)CC3)n[nH]c2C1]

has been advanced to Phase 3 trials for STGD1 (Belite Bio 2020) Belite Bio reported positive results from a Phase 1b trial and 6-month interim safety and efficacy data for an ongoing two-year Phase 2 trial involving early-onset STGD1 adolescents chronically dosed with tinlarebant, and a Phase 3 trial with adolescent STGD1 patients has been initiated. However, this class of compounds does not address the issue of potential mechanism-based ocular AEs associated with serum RBP4 reduction.

### 2. C20-D3-Retinol

A critical step in bisretinoid synthesis is spontaneous dimerization of retinaldehydes conjugated to phosphatidylethanolamine (PE) via Schiff base formation in photoreceptor membranes (Washington, I. et al. 2016). A rate-determining step in retinaldehyde dimerization is the cleavage of carbon-hydrogen bonds at C20 of the retinaldehyde-PE Schiff base via a [1,6]-hydride shift (Kaufman, Y. et al. 2011).

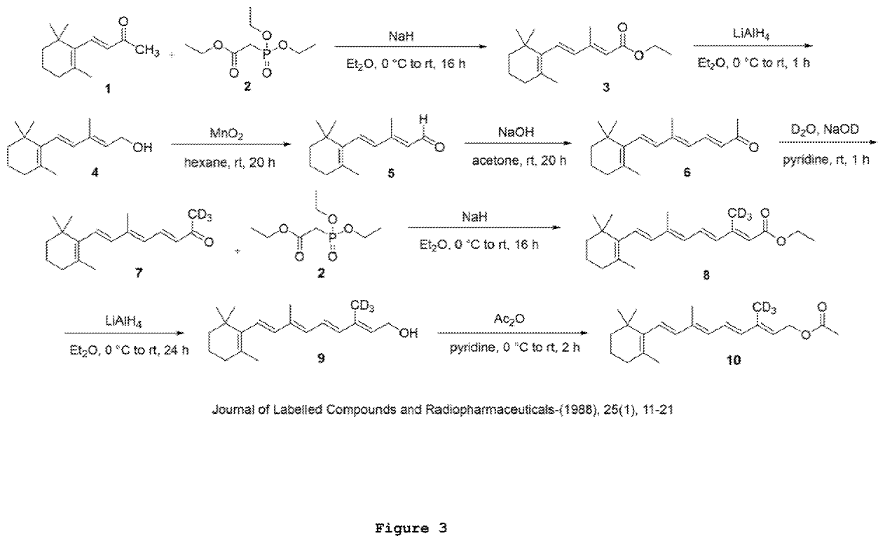

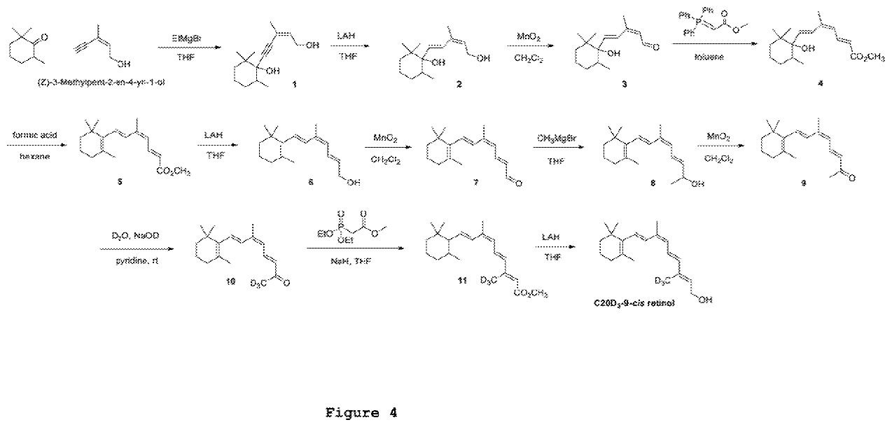

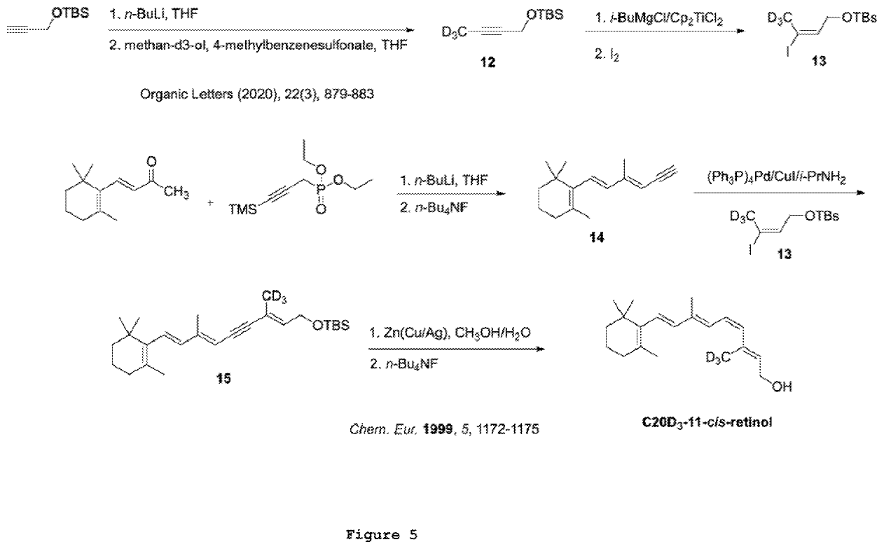

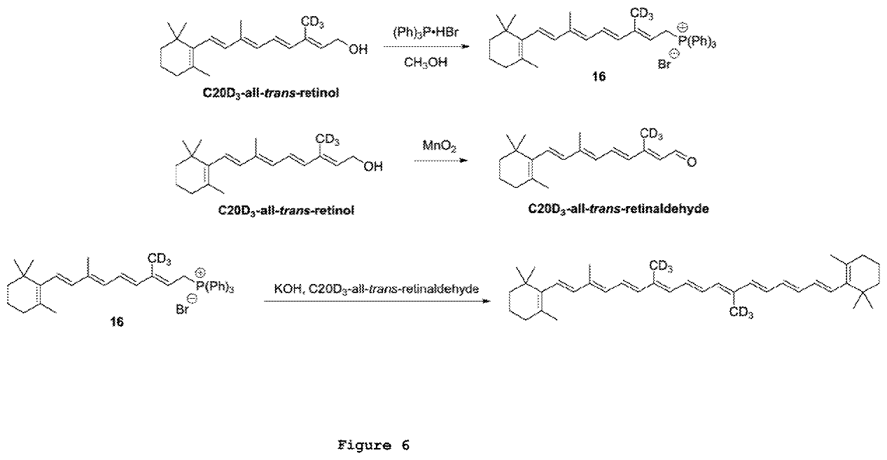

Deuterated retinoids have been used in humans for decades as traces in studies of vitamin A metabolism (Haskell, M. J. et al. 1999; Haskell, M. J. et al. 1997). FIGS. 3 through 6 show, respectively, how C20-D3-retinol, C20-D3-retinyl acetate, C20-D3-9-cis-retinol, C20-D3-11-cis-retinol, and C20-D3-C20′-D3-β-carotene may be synthesized. Introduction of deuterium at C20 of vitamin A results in a kinetic isotope effect that slows the formation of retinaldehyde dimers in vivo and in vitro (Kaufman, Y. et al. 2011; Ma, L. et al. 2011). In in vitro experiments, C20-D3-retinaldehyde formed dimers 12-times less rapidly than unlabeled retinaldehyde, while bisretinoid A2E formation was reduced by 7-fold (Kaufman, Y. et al. 2011). C20-D3-retinyl acetate was effective in inhibiting bisretinoid synthesis and normalization of complement system dysregulation in the retina of Abca4^{-/-} mice, a model of Stargardt disease (STGD1) (Charbel, I. P. et al. 2015).

Evaluation of C20-D3-retinyl acetate (under the name ALK-001) in clinical trials for dry AMD and STGD1 is ongoing (Petrukhin, K. 2020). Standing alone, the bisretinoid lowering efficacy of ALK-001 may depend on dietary restrictions in consuming standard vitamin A-containing food (Petrukhin, K. 2013). Moreover, an emerging view of dry AMD and STGD1 pathogenesis underscores the independent role of retinaldehyde toxicity in the disease pathology (Maeda, A. et al. 2012; Gliem, M. et al. 2016; Smith, R. T. et al. 2013; Sparrow, J. R. et al. 2013).

### 3. Antibody-Drug Conjugate (ADC) Systems

An antibody-drug conjugate (ADC) is a class of therapeutics that combines the selectivity of monoclonal antibodies (mAbs) with the potency of cytotoxic drugs. It is designed to target specific cells, such as cancer cells, while minimizing the impact on healthy cells, thereby enhancing the efficacy and reducing the side effects of traditional chemotherapy (Su, Z. et al. 2021; Alas, M. et al. 2021; Tsuchikama, K. & An, Z. 2018).

The structure of an ADC typically consists of three main components:
Monoclonal Antibody (mAb): The mAb used in an ADC is designed to recognize and bind to a specific antigen that is overexpressed or selectively present on the target cells, such as tumor cells. The antibody provides the specificity and targeting capability of the ADC.
Linker: The linker serves as a bridge between the mAb and the cytotoxic drug. It is engineered to be stable in circulation but capable of releasing the drug payload selectively inside the target cells. The linker plays a crucial role in determining the release kinetics and stability of the ADC.
Cytotoxic Drug Payload: The cytotoxic drug, also known as the payload, is the pharmacologically active component of the ADC that exerts a toxic effect on the target cells. The drug is conjugated to the mAb via the linker. The choice of the cytotoxic drug depends on the therapeutic target and desired mechanism of action.

The mechanism of action of an ADC involves a series of steps:
Target Binding: The ADC is administered systemically, and the mAb component recognizes and binds specifically to the target antigen present on the surface of the cancer cells.
Internalization: Once the ADC binds to the target cells, it is internalized through receptor-mediated endocytosis, forming an endosome within the cell.
Intracellular Processing: Within the endosome, the linker is designed to be cleaved in response to specific conditions present in the target cell's environment, such as low pH or enzymatic activity. This cleavage releases the cytotoxic drug payload from the ADC.
Cytotoxic Effect: After release, the cytotoxic drug enters the cytoplasm or other cellular compartments, where it exerts its toxic effect. The drug may disrupt cellular processes, inhibit DNA replication, promote apoptosis (programmed cell death), or interfere with microtubule formation, depending on the specific drug used.

The goal of ADC therapy is to deliver the cytotoxic drug specifically to the target cells, reducing off-target effects and minimizing damage to healthy tissues. By leveraging the targeting capabilities of monoclonal antibodies, ADCs offer the potential for improved therapeutic efficacy with reduced systemic toxicity compared to conventional chemotherapy.

The co-drug designs of the present disclosure take advantage of a wide variety of established linkers used in antibody-drug conjugate (ADC) systems for rapid payload release via acidic pH or proteolytic cleavage (Su, Z. et al. 2021; Alas, M. et al. 2021; Tsuchikama, K. & An, Z. 2018). For example, a co-drug containing C20-D3-retinol linked to its core via a low-pH sensitive carbonate, silyl ether, or ester linker will rapidly cleave in the stomach and upper GI tract, readily releasing C20-D3-retinol for intestinal absorption and chylomicron packaging. Selective RBP4 antagonists of the co-drugs may be attached to their respective cores via ester, carbamate, or hemiaminal linkages, which will undergo rapid chemical and enzymatic hydrolysis in the GI tract.

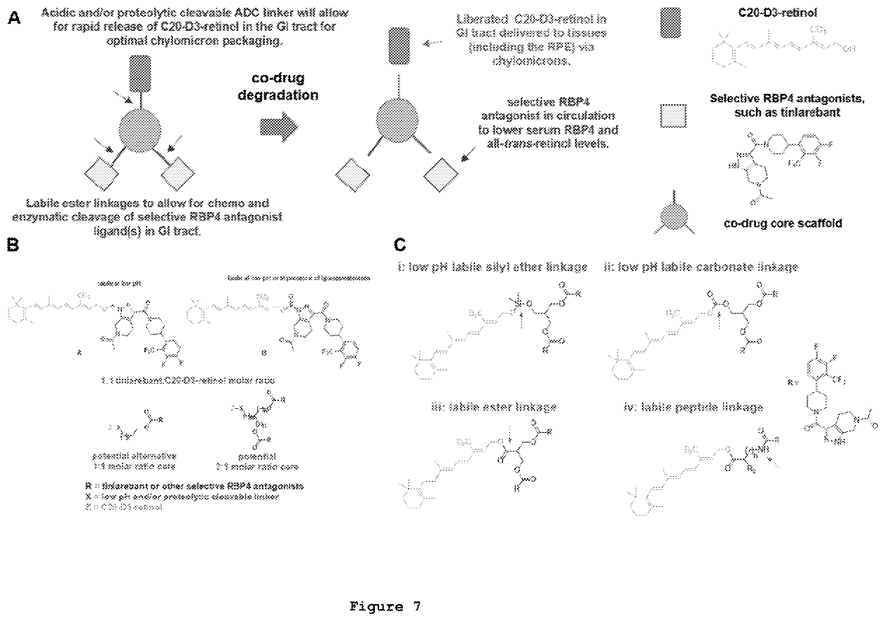

FIG. 7 describes key design principles of the co-drug platform of the present disclosure, using tinlarebant (BPN-14697; LBS-008) as an example of a selective RBP4 antagonist. Given that excessive amounts of dietary vitamin A may lead to liver damage (Park, J. et al. 2020; Nollevaux, M. C. et al. 2006), it is important to have an optimal ratio of a selective RBP4 antagonist to C20-D3-retinol in a co-drug to avoid delivering excessive retinol while providing an efficacious dose of the selective RBP4 antagonist. For efficacy, a 5-20 mg daily human dose of a selective RBP4 antagonist is required. On the other hand, daily consumption of up to 3 mg of retinol is safe.

### Proposed Tinlarebant-C20D3-all-Trans-Retinol Co-Drugs

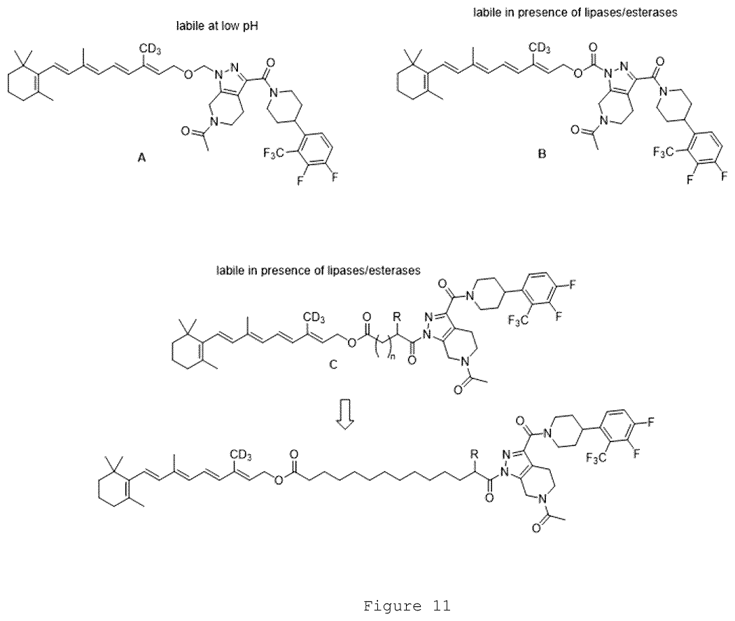

The present invention provides proposed BPN-14634-C20D3-all-trans-Retinol Co-Drugs A, B, C and D. (See FIG. 11). Relevant synthesis may be found in WO2015179441 A2; WO2003059878 A2; WO2023018643 A1; and WO2018060481 A1.

### Proposed Tinlarebant-C20D3-all-Trans-Retinol Co-Drug A

The present invention provides a proposed Tinlarebant-C20D3-all-trans-Retinol Co-Drug A having the following structure:

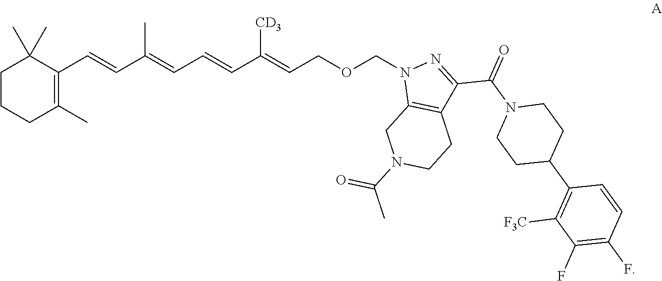 [SMILES: [2H]C([2H])([2H])C(/C=C/C=C(C)/C=C/C1=C(C)CCCC1(C)C)=C\COCn1nc(C(=O)N2CCC(c3ccc(F)c(F)c3C(F)(F)F)CC2)c2c1CN(C(C)=O)CC2]

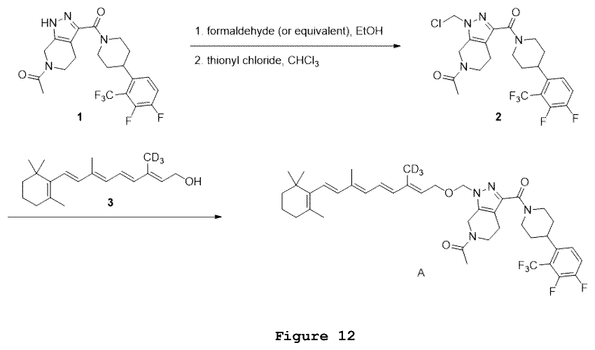

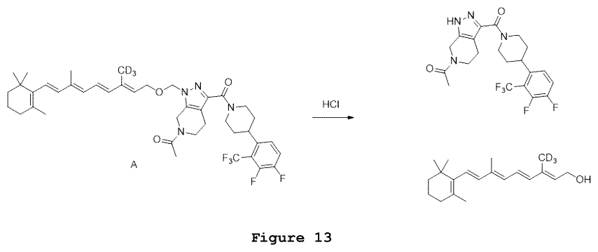

The proposed synthesis steps are described in FIG. 12. The connection point in A is unstable at low pH; and should degrade in the stomach to release tinlarebant and C20D3-all-trans-retinol. (See FIG. 13). (Heterocycles (1986), 24(8), 2233-7; Pharmazie (1980), 35(12), 746-8; WO2021113436; and WO2002041835).

### Proposed Tinlarebant-C20D3-all-Trans-Retinol Co-Drug B:

The present invention provides a proposed Tinlarebant-C20D3-all-trans-Retinol Co-Drug B having the following structure:

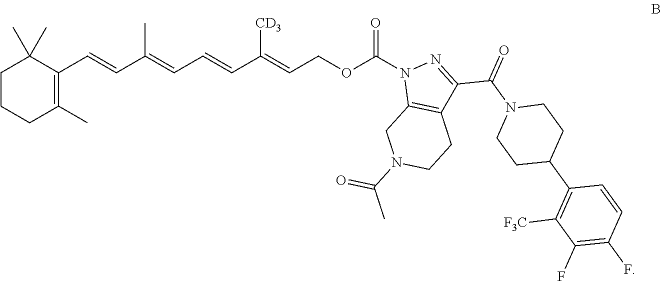 [SMILES: [2H]C([2H])([2H])C(/C=C/C=C(C)/C=C/C1=C(C)CCCC1(C)C)=C\COC(=O)n1nc(C(=O)N2CCC(c3ccc(F)c(F)c3C(F)(F)F)CC2)c2c1CN(C(C)=O)CC2]

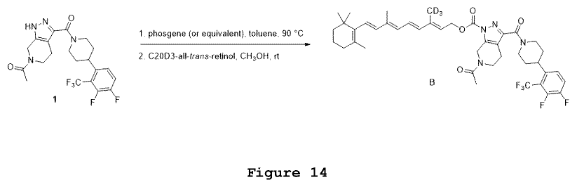

The proposed synthesis steps are described in FIG. 14. The connection point (cabamate) in B is predicted to be labile in the presences of esterases and proteases. (Croatica Chemica Acta (2003), 76(3), 217-228).

### Proposed Tinlarebant-C20D3-all-Trans-Retinol Co-Drug C:

The present invention provides a proposed Tinlarebant-C20D3-all-trans-Retinol Co-Drug C having the following structure:

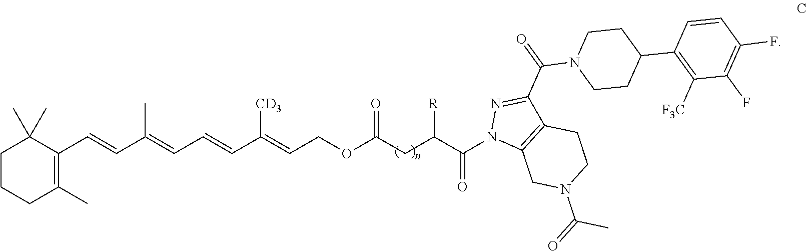

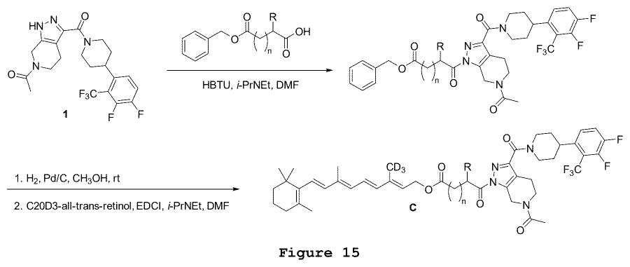

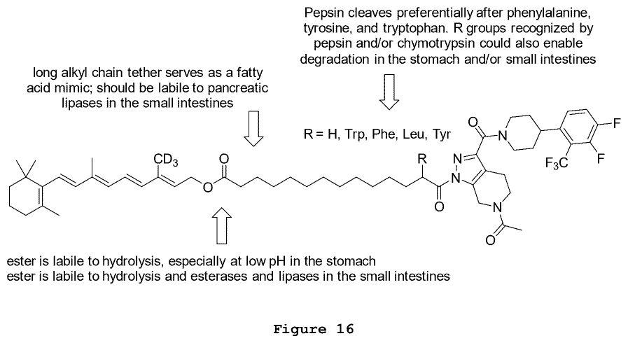

The proposed synthesis steps are described in FIG. 15. The long alkyl chain tether serves as a fatty acid mimic and should be labile to pancreatic lipases in the small intestines. Various R groups are shown in FIG. 16. Pepsin cleaves preferentially after phenylalanine, tyrosine, and tryptophan. R groups recognized by pepsin and/or chymotrypsin could also enable degradation in the stomach and/or small intestines. The ester in Co-Drug C is labile to hydrolysis especially at low pH in the stomach and is labile to esterases and lipases in the small intestines.

### Proposed Tinlarebant-C20D3-all-Trans-Retinol Co-Drug D

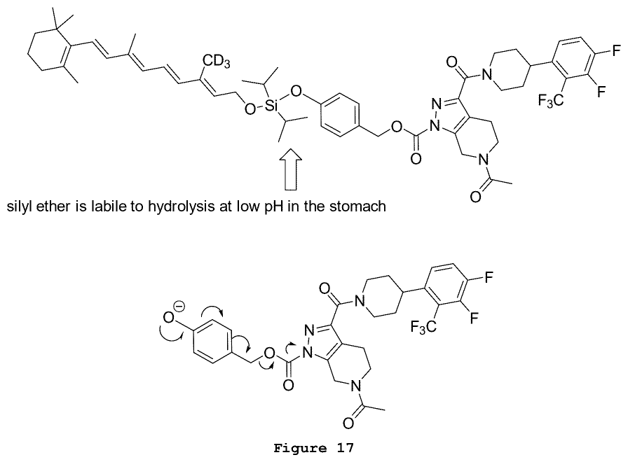

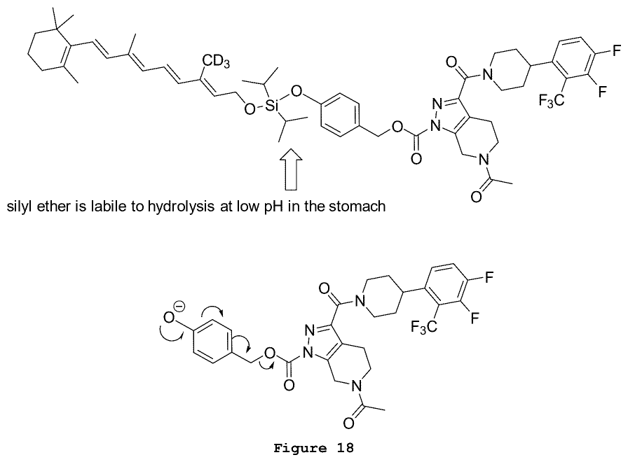

The present invention provides a proposed Tinlarebant-C20D3-all-trans-Retinol Co-Drug D having the following structure (FIGS. 17 and 18):

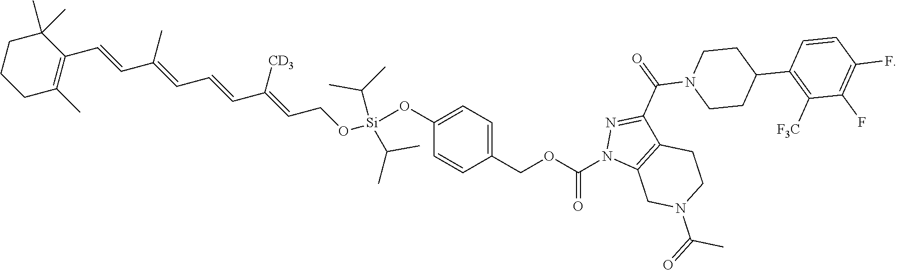 [SMILES: [2H]C([2H])([2H])C(/C=C/C=C(C)/C=C/C1=C(C)CCCC1(C)C)=C\CO[Si](Oc1ccc(COC(=O)n2nc(C(=O)N3CCC(c4ccc(F)c(F)c4C(F)(F)F)CC3)c3c2CN(C(C)=O)CC3)cc1)(C(C)C)C(C)C]

The silyl ether is labile to hydrolysis at low pH in the stomach.

### Proposed BPN-14634-C20D3-all-Trans-Retinol Co-Drugs

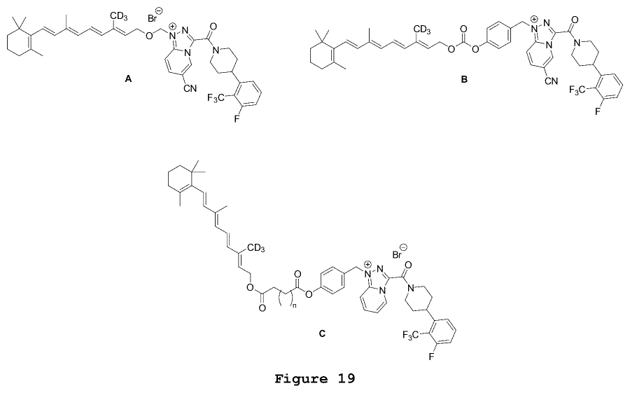

The present invention provides proposed BPN-14634-C20D3-all-trans-Retinol Co-Drugs A, B, and C. (See FIG. 19). (Chemical Papers (1985), 39(3), 413-27).

### Proposed BPN-14634-C20D3-all-Trans-Retinol Co-Drug A

The present invention provides a proposed BPN-14634-C20D3-all-trans-Retinol Co-Drug A having the following structure:

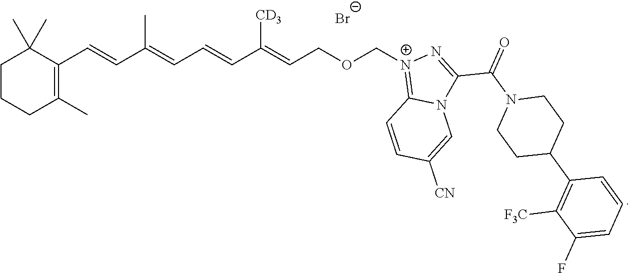 [SMILES: [2H]C([2H])([2H])C(/C=C/C=C(C)/C=C/C1=C(C)CCCC1(C)C)=C\COC[n+]1nc(C(=O)N2CCC(c3cccc(F)c3C(F)(F)F)CC2)n2cc(C#N)ccc21 ; [Br-]]

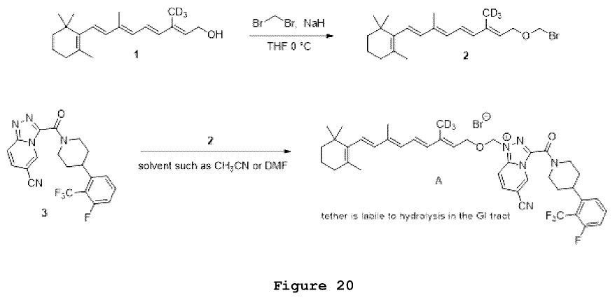

The proposed synthesis steps were described in FIG. 20. (CN 101671369; and CN105001193).

### Proposed BPN-14634-C20D3-all-Trans-Retinol Co-Drug B

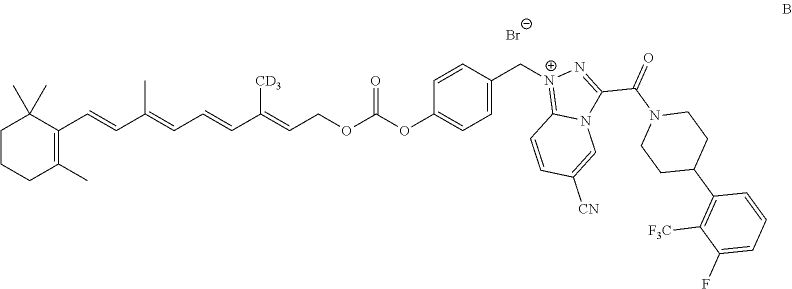 [SMILES: [Br-] ; [2H]C([2H])([2H])C(/C=C/C=C(C)/C=C/C1=C(C)CCCC1(C)C)=C\COC(=O)Oc1ccc(C[n+]2nc(C(=O)N3CCC(c4cccc(F)c4C(F)(F)F)CC3)n3cc(C#N)ccc32)cc1]

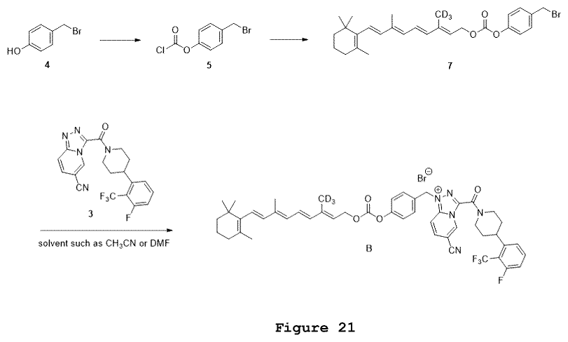

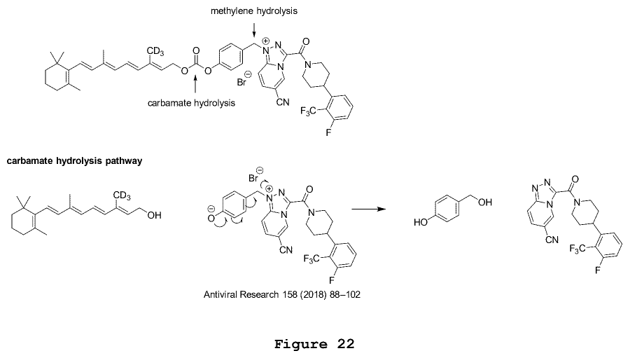

The proposed synthesis steps were described in FIG. 21. (Chemical Papers (1985), 39(3), 413-27). Co-Drug B is tether based on nucleotide prodrug strategy and BPN-14634 can be liberated either via direct hydrolysis of the methylene linker or via degradation of the linker after carbamate hydrolysis (carbamate pathway). (FIG. 22, Antiviral Research 158 (2018) 88-102).

### Proposed BPN-14634-C20D3-all-Trans-Retinol Co-Drug C

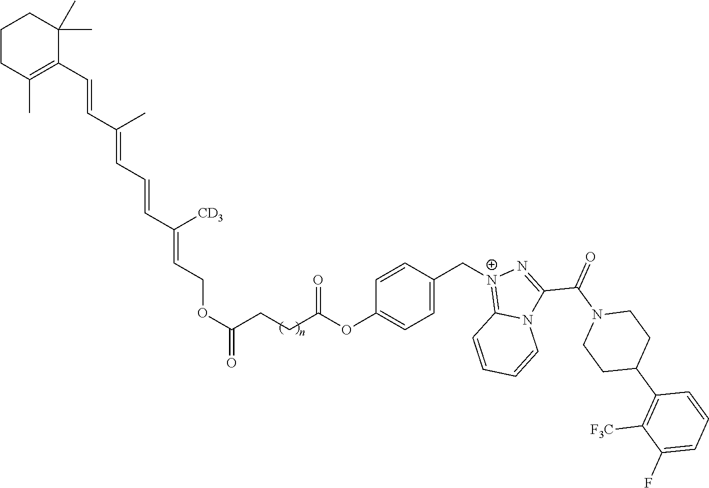

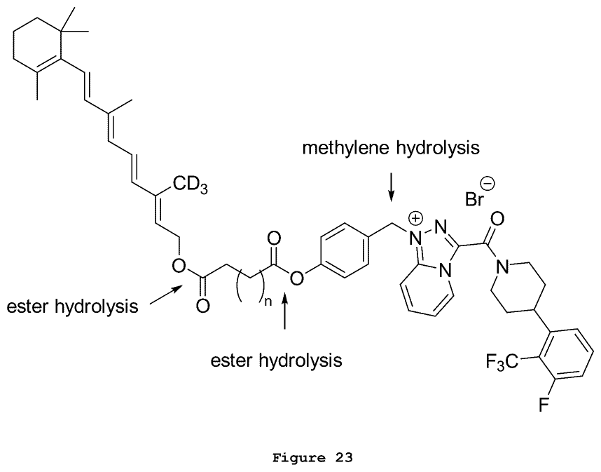

Co-Drug C is tether based on nucleotide prodrug strategy and BPN-14634 can be liberated either via direct hydrolysis of the methylene linker or via degradation of the linker after ester hydrolysis (similar to carbamate pathway of Co-Drug B). (FIG. 23).

One embodiment of the present disclosure is a co-drug that: contains an optimal molar ratio of a selective RBP4 antagonist to C20-D3-retinol (e.g., within a 1:1 to 5:1 range or within a 1:1 to 1:5 range); and can readily degrade to liberate the selective RBP4 antagonist and the C20-D3-retinol in the GI tract for chylomicron packaging and delivery. A wide variety of established linkers used in antibody-drug conjugate (ADC) systems for rapid payload release via acidic pH or proteolytic cleavage may be used for this purpose (Su, Z. et al. 2021; Alas, M. et al. 2021; Tsuchikama, K. & An, Z. 2018). For example, a co-drug containing C20-D3-retinol linked to a core via a carbonate or silyl ether linker can rapidly cleave in the stomach and upper GI tract, readily releasing C20-D3-retinol for intestinal absorption and chylomicron packaging. The selective RBP4 antagonists of the co-drugs can be attached to their respective cores via ester, carbamate, or hemiaminal linkages, which will undergo rapid chemo- and enzymatic hydrolysis in the GI tract.

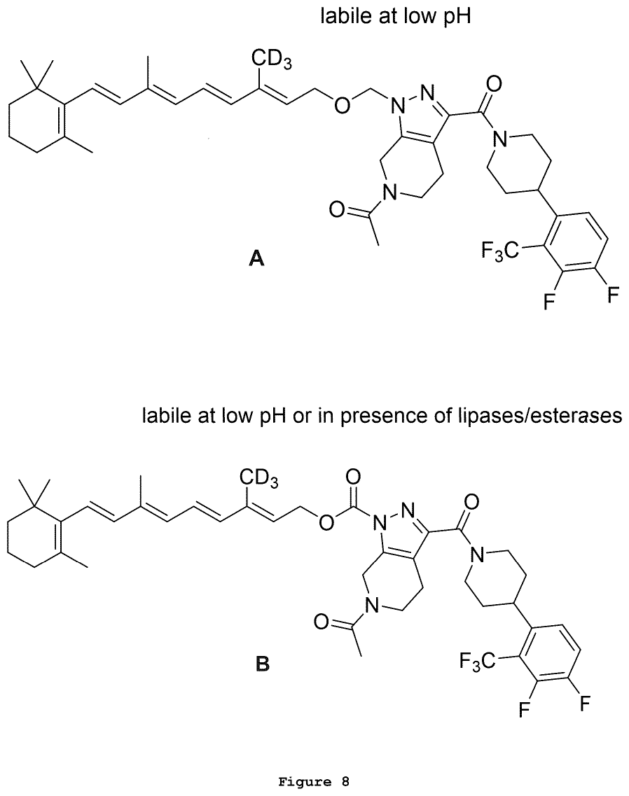

As the design of co-drugs requires that they rapidly degrade within the GI tract, one of ordinary skill in the art would know that the key parameters to be monitored include: (1) chemical stability time course under various pH ranges within a buffered aqueous environment and monitoring of rate of C20-D3-retinol release and selective RBP4 antagonist release; (2) aqueous solubility; and (3) careful monitoring of the oral absorption and PK of both payloads. These data may be used in iterative design cycles with various diverse core scaffolds and cleavable linkers to avoid PK variability. FIGS. 8 and 11 show examples of co-drugs of tinlarebant and C20-D3-all-trans-retinol. A selective RBP4 antagonist and a C20-D3-visual-chromophore-producing compounnd may also be chemically bonded directly to each other to form a co-drug, namely, without involving an intervening linker.

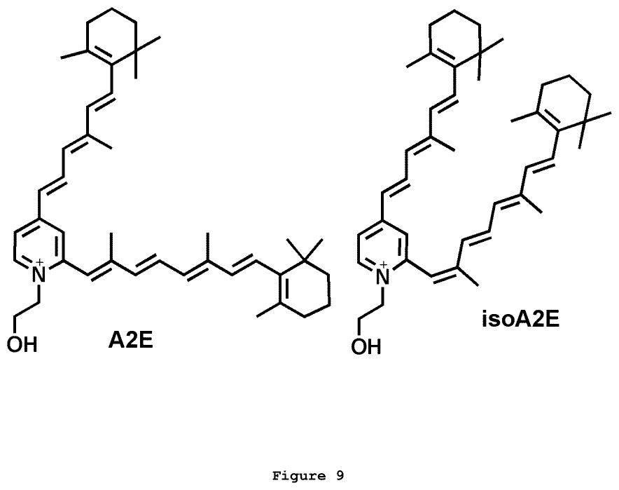

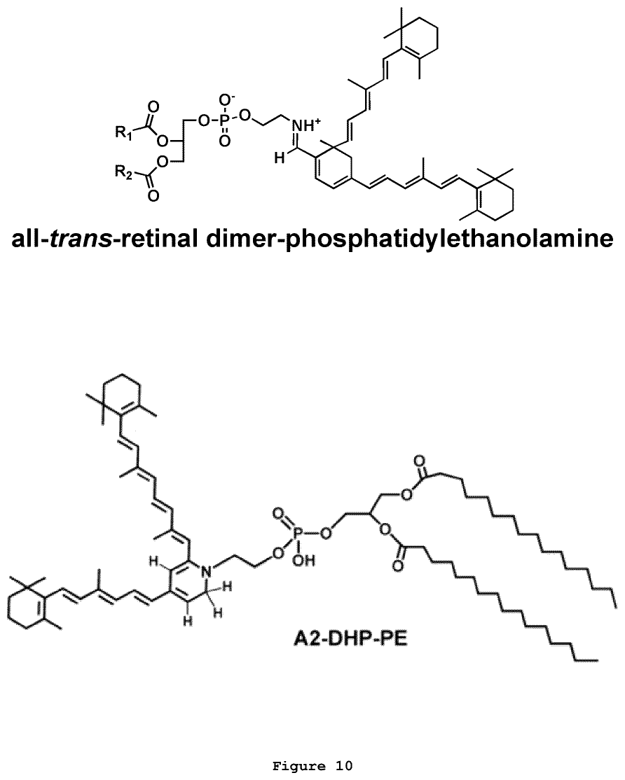

As used herein, “bisretinoid lipofuscin” is lipofuscin containing a cytotoxic bisretinoid. Cytotoxic bisretinoids include but are not necessarily limited to A2E, isoA2E, atRAL di-PE (all-trans-retinal dimer-phosphatidylethanolamine), and A2-DHP-PE (A2-dihydropyridine-phosphatidylethanolamine) (FIGS. 9 and 10). Bisretinoid-mediated macular degeneration may comprise the accumulation of lipofuscin deposits in the retinal pigment epithelium.

As used herein, “combination” means an assemblage of reagents for use in therapy either by simultaneous or contemporaneous administration. Simultaneous administration refers to administration of an admixture (whether a true mixture, a suspension, an emulsion, or other physical combination) of the first compound and the second compound. In this case, the combination may be the admixture or separate containers of the first compound and the second compound that are combined just prior to administration. Contemporaneous administration refers to the separate administration of the first compound and the second compound at the same time, or at times sufficiently close together that an additive or preferably synergistic activity relative to the activity of either the first compound or the second compound alone is observed.

As used herein, “concomitant administration” or “administering concomitantly” means the administration of two agents given in close enough temporal proximity to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time.

The present disclosure provides a compound having the general structure (P-)ₐM(-Q)_{b} or P(-Q)_{b}, wherein: P represents a single-valence group formed by eliminating a hydrogen atom bonded to a heteroatom or by eliminating a hydroxyl group from a selective RBP4 antagonist;
a is 1, 2, 3, 4, or 5;
Q represents a single-valence group formed by eliminating a hydrogen atom bonded to a heteroatom or by eliminating a hydroxyl group from a C20-D3-visual-chromophore-producing compound;
b is 1, 2, 3, 4, or 5;
M represents an (a+b)-valence group comprising carbon, hydrogen, nitrogen, and oxygen atoms;
each P in (P-)ₐM(-Q)_{b} is independently bonded to M via an ester, carbonyl, peptide, carbamate, or hemiaminal linkage that is cleavable at an acidic pH or is enzymatically cleavable;
each Q in (P-)ₐM(-Q)_{b} is independently bonded to M via a carbonate, carbonyl, peptide, silyl ether, or ester linkage that is cleavable at an acidic pH or is enzymatically cleavable;
each Q in P(-Q)_{b} is independently bonded to P via an ester, carbonyl, peptide, carbamate, or hemiaminal linkage that is cleavable at an acidic pH or is enzymatically cleavable;
the selective RBP4 antagonist is a compound having the structure:

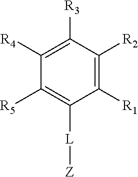

wherein L is a linking group having the structure

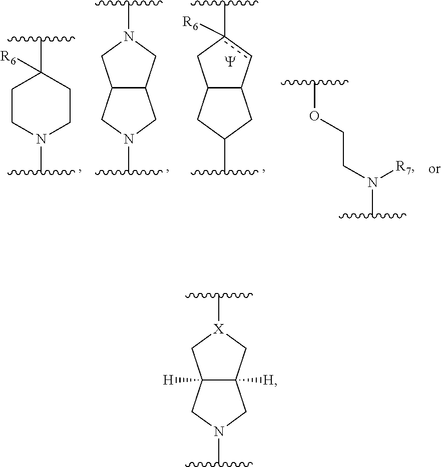

and
Z is a group having the structure

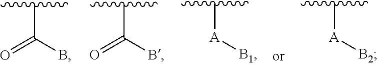

wherein
R₁, R₂, R₃, R₄, and R₅ are each independently H, halogen, CF₃, C₁-C₄ alkyl, aryl, or heteroaryl;
R₆ is H, OH, or halogen, or is absent;
ψ is absent or present, and when present, is a bond;
B is a substituted or unsubstituted heterobicycle, pyridazine, pyrazole, pyrazine, thiadiazole, or triazole, wherein
the heterobicycle is other than chloro-substituted indole; and
the pyrazole, when substituted, is substituted with other than trifluoromethyl;
   B′ is a substituted or unsubstituted phenyl, pyridine, pyrimidine, benzyl, pyrrolidine, sulfolane, oxetane, CO₂H, or (C₁-C₄ alkylenyl)-CO₂H, wherein
the substituted phenyl is substituted with other than trifluoromethyl or 3-(methyl carboxylate),
the substituted pyridine is substituted with other than trifluoromethyl,
the substituted pyrrolidine is substituted with other than hydroxamic acid, and
the substituted or unsubstituted pyrrolidine is bound to the carbonyl through a carbon-carbon bond;
   A is absent or present, and when present, is

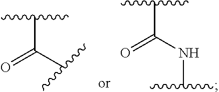

B₁ is a substituted or unsubstituted monocycle, bicycle, heteromonocycle, heterobicycle, benzyl, CO₂H, or (C₁-C₄ alkylenyl)-CO₂H,
wherein when B₁ is CO₂H, then A is present and is

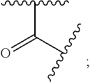 [SMILES: *C(*)=O]

R₇ is alkyl;

### X is N or CR_{B},

wherein R₈ is H, OH, or halogen; and
   B₂ has the structure:

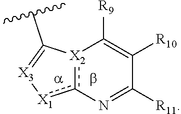

wherein
α and β are each a bond that is present or absent;
X₁ is N, NH, or NR₉₉,
wherein R₉₉ is alkyl, alkenyl, or alkynyl;

### X₂ is C or N;

### X₃ is CH or N;

R₉, R₁₀, and R₁₁ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
X₁, X₂, and X₃ are each N, α is present, and β is absent; or
X₁ is NH, X₂ is C, X₃ is CH, α is absent, and β is present; or
X₁ is N, X₂ is N, X₃ is CH, α is present, and β is absent; or
X₁ is NH or NR₉₉, X₂ is C, X₃ is N, α is absent, and β is present, wherein when X₁ is NH, X₂ is C, X₃ is N, α is absent, and β is present, then one of R₉, R₁₀, and R₁₁ is other than H;
   or
   B₂ has the structure:

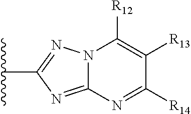

wherein
R₁₂, R₁₃, and R₁₄ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
or a pharmaceutically acceptable salt thereof; and
the C20-D3-visual-chromophore-producing compound is selected from the group consisting of C20-D3-retinol, C20-D3-9-cis-retinol, C20-D3-11-cis-retinol, and C20-D3-C20′-D3-B-carotene.

The present disclosure provides a compound having the general structure (P-)ₐM(-Q)_{b} or P(-Q)_{b}, wherein:
P represents a single-valence group formed by eliminating a hydrogen atom bonded to a heteroatom or by eliminating a hydroxyl group from a selective RBP4 antagonist;
a is 1, 2, 3, 4, or 5;
Q represents a single-valence group formed by eliminating a hydrogen atom bonded to a heteroatom or by eliminating a hydroxyl group from a C20-D3-visual-chromophore-producing compound;
b is 1, 2, 3, 4, or 5;
M represents an (a+b)-valence group comprising carbon, hydrogen, and oxygen atoms;
each P in (P-)ₐM(-Q)_{b} is independently bonded to M via an ester, carbamate, or hemiaminal linkage that is cleavable at an acidic pH or is enzymatically cleavable;
each Q in (P-)ₐM(-Q)_{b} is independently bonded to M via a carbonate, silyl ether, or ester linkage that is cleavable at an acidic pH or is enzymatically cleavable;
each Q in P(-Q)_{b} is independently bonded to P via an ester, carbamate, or hemiaminal linkage that is cleavable at an acidic pH or is enzymatically cleavable;
the selective RBP4 antagonist is a compound having the structure:

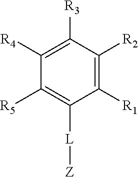

wherein L is a linking group having the structure

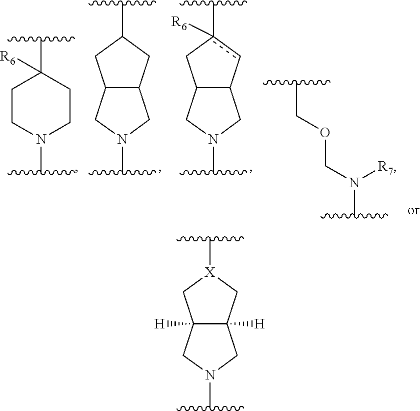

and
Z is a group having the structure

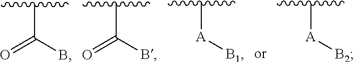

wherein
R₁, R₂, R₃, R₄, and R₅ are each independently H, halogen, CF₃, C₁-C₄ alkyl, aryl, or heteroaryl;
R₆ is H, OH, or halogen, or is absent;
ψ is absent or present, and when present, is a bond;
B is a substituted or unsubstituted heterobicycle, pyridazine, pyrazole, pyrazine, thiadiazole, or triazole, wherein
   the heterobicycle is other than chloro-substituted indole; and
   the pyrazole, when substituted, is substituted with other than trifluoromethyl;
      B′ is a substituted or unsubstituted phenyl, pyridine, pyrimidine, benzyl, pyrrolidine, sulfolane, oxetane, CO₂H, or (C₁-C₄ alkylenyl)-CO₂H, wherein
the substituted phenyl is substituted with other than trifluoromethyl or 3-(methyl carboxylate),
the substituted pyridine is substituted with other than trifluoromethyl,
the substituted pyrrolidine is substituted with other than hydroxamic acid, and
the substituted or unsubstituted pyrrolidine is bound to the carbonyl through a carbon-carbon bond;
   A is absent or present, and when present, is

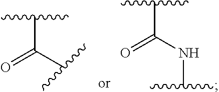

B₁ is a substituted or unsubstituted monocycle, bicycle, heteromonocycle, heterobicycle, benzyl, CO₂H, or (C₁-C₄ alkylenyl)-CO₂H,
wherein when B₁ is CO₂H, then A is present and is

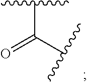 [SMILES: *C(*)=O]

R₇ is alkyl;

### X is N or CR_{B},

wherein R₈ is H, OH, or halogen; and
   B₂ has the structure:

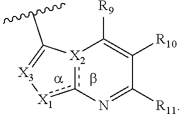

wherein
α and β are each a bond that is present or absent;
X₁ is N, NH, or NR₉₉,
wherein R₉₉ is alkyl, alkenyl, or alkynyl;

### X₂ is C or N;

### X₃ is CH or N;

R₉, R₁₀, and R₁₁ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃, wherein
X₁, X₂, and X₃ are each N, α is present, and β is absent; or
X₁ is NH, X₂ is C, X₃ is CH, α is absent, and β is present; or
X₁ is N, X₂ is N, X₃ is CH, α is present, and β is absent; or
X₁ is NH or NR₉₉, X₂ is C, X₃ is N, α is absent, and β is present, wherein when X₁ is NH, X₂ is C, X₃ is N, α is absent, and β is present, then one of R₉, R₁₀, and R₁₁ is other than H;
   or
   B₂ has the structure:

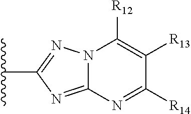

wherein
R₁₂, R₁₃, and R₁₄ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
or a pharmaceutically acceptable salt thereof; and
the C20-D3-visual-chromophore-producing compound is selected from the group consisting of C20-D3-retinol, C20-D3-9-cis-retinol, C20-D3-11-cis-retinol, and C20-D3-C20′-D3-(3-carotene.

In some embodiments, the selective RPB4 antagonist is the compound wherein R₁, R₂, R₃, R₄, and R₅ are each independently H, halogen, CF₃, or C₁-C₄ alkyl.

In some embodiments, the selective RPB4 antagonist is the compound wherein L is

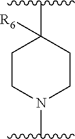

### and Z is

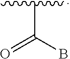

In some embodiments, the selective RBP4 antagonist is the compound wherein L is

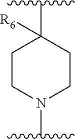

### and Z is

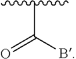

In some embodiments, the selective RBP4 antagonist is the compound wherein
L is

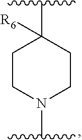

Z is

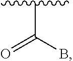

two or more of R₁, R₂, R₃, R₄, and R₅ are other than H, and
when
   R₁ is CF₃, R₂ is H, R₃ is F, R₄ is H, and R₅ is H, or
   R₁ is H, R₂ is CF₃, R₃ is H, R₄ is CF₃, and R₅ is H, or
   R₁ is Cl, R₂ is H, R₃ is H, R₄ is F, and R₅ is H, or
   R₁ is CF₃, R₂ is H, R₃ is F, R₄ is H, and R₅ is H, or
   R₁ is CF₃, R₂ is F, R₃ is H, R₄ is H, and R₅ is H, or
   R₁ is Cl, R₂ is F, R₃ is H, R₄ is H, and R₅ is H,
then B is other than

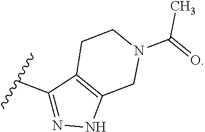 [SMILES: *c1n[nH]c2c1CCN(C(C)=O)C2]

In some embodiments, the selective RBP4 antagonist is the compound wherein L is

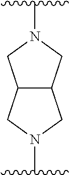 [SMILES: *N1CC2CN(*)CC2C1]

### and Z is

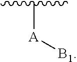

In some embodiments, the selective RBP4 antagonist is the compound wherein
L is

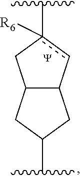

Z is

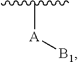

R₆ is absent or present, and when present, is H, OH, or halogen, and
when ψ is present, then R₆ is absent, and when ψ is absent, then R₆ is present.

In some embodiments, the selective RBP4 antagonist is the compound wherein L is

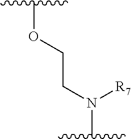

### and Z is

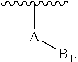

In some embodiments, the selective RBP4 antagonist is the compound wherein L is

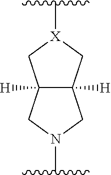

### and Z is B₂.

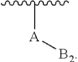

In some embodiments, the selective RBP4 antagonist is the compound wherein
L is

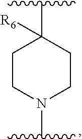

Z is B₂

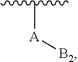

R₆ is H, and
A is

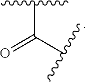 [SMILES: *C(*)=O]

In some embodiments, the selective RBP4 antagonist is the compound wherein L is

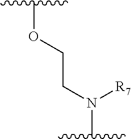

### and Z is

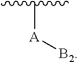

In some embodiments, the selective RBP4 antagonist is

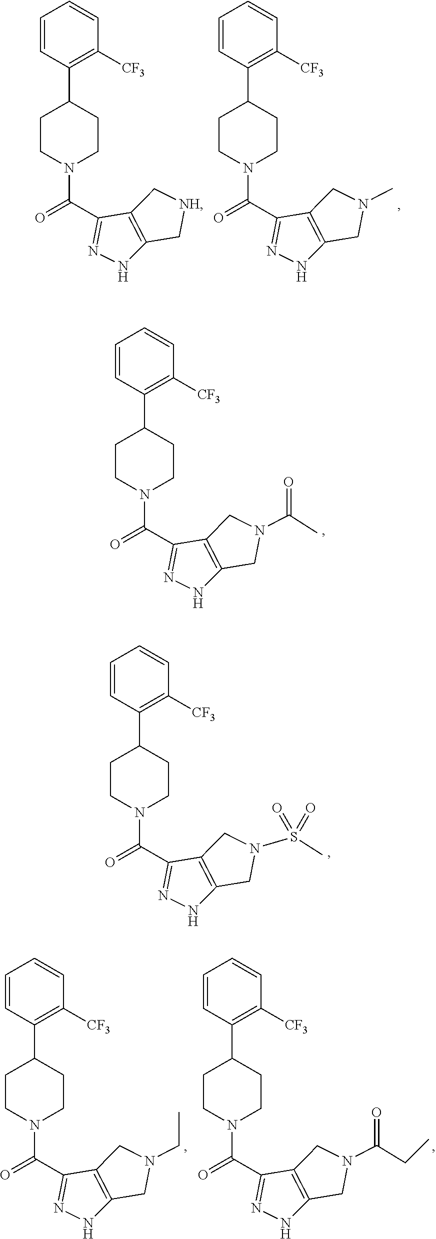

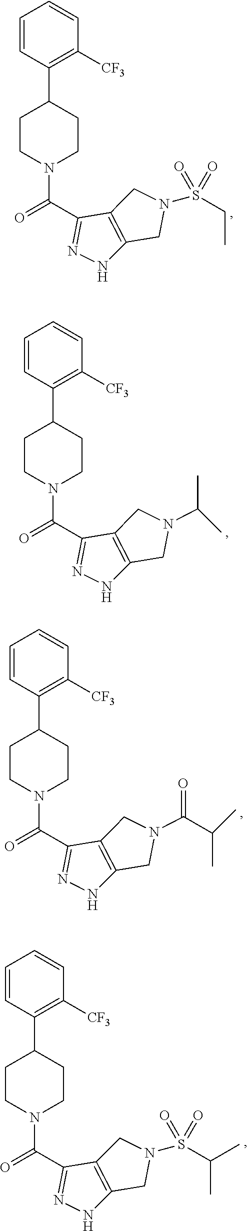 [SMILES: CCS(=O)(=O)N1Cc2[nH]nc(C(=O)N3CCC(c4ccccc4C(F)(F)F)CC3)c2C1 ; CC(C)N1Cc2[nH]nc(C(=O)N3CCC(c4ccccc4C(F)(F)F)CC3)c2C1 ; CC(C)C(=O)N1Cc2[nH]nc(C(=O)N3CCC(c4ccccc4C(F)(F)F)CC3)c2C1 ; CC(C)S(=O)(=O)N1Cc2[nH]nc(C(=O)N3CCC(c4ccccc4C(F)(F)F)CC3)c2C1]

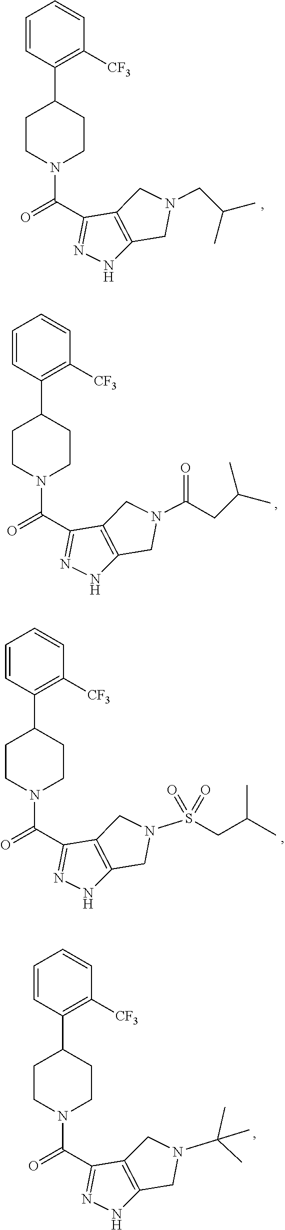 [SMILES: CC(C)CN1Cc2[nH]nc(C(=O)N3CCC(c4ccccc4C(F)(F)F)CC3)c2C1 ; CC(C)CC(=O)N1Cc2[nH]nc(C(=O)N3CCC(c4ccccc4C(F)(F)F)CC3)c2C1 ; CC(C)CS(=O)(=O)N1Cc2[nH]nc(C(=O)N3CCC(c4ccccc4C(F)(F)F)CC3)c2C1 ; CC(C)(C)N1Cc2[nH]nc(C(=O)N3CCC(c4ccccc4C(F)(F)F)CC3)c2C1]

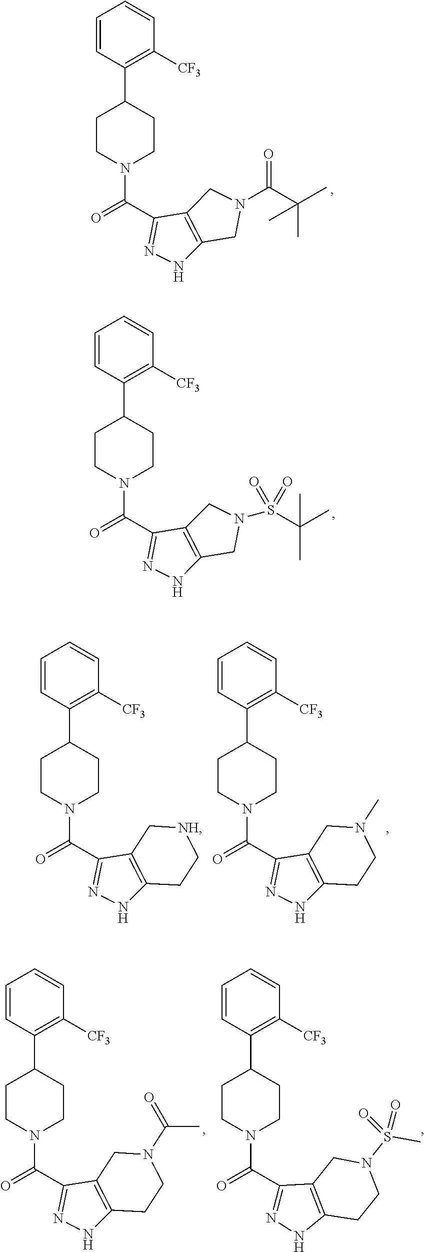

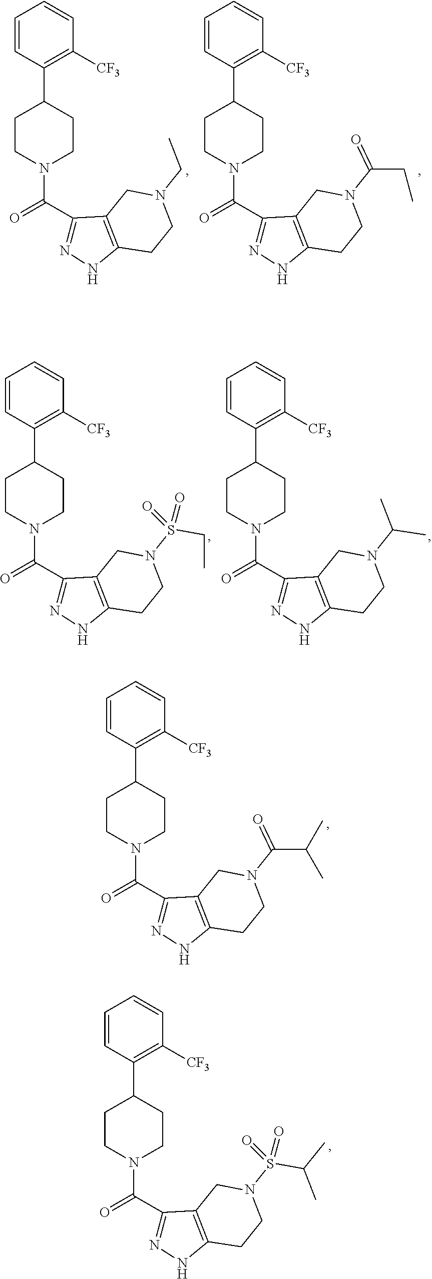

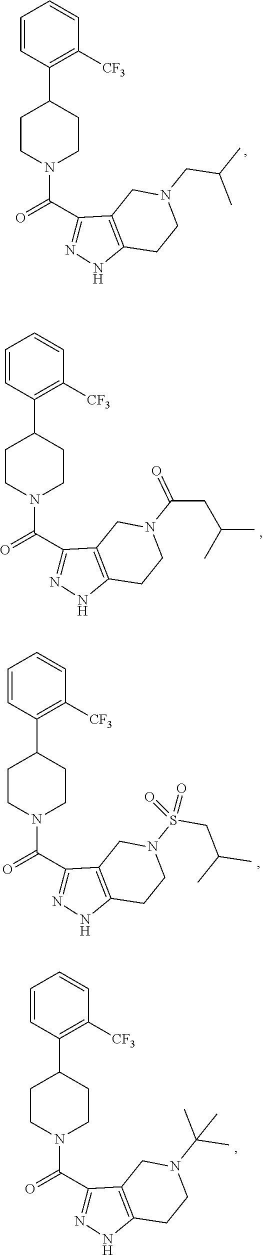 [SMILES: CC(C)CN1CCc2[nH]nc(C(=O)N3CCC(c4ccccc4C(F)(F)F)CC3)c2C1 ; CC(C)CC(=O)N1CCc2[nH]nc(C(=O)N3CCC(c4ccccc4C(F)(F)F)CC3)c2C1 ; CC(C)CS(=O)(=O)N1CCc2[nH]nc(C(=O)N3CCC(c4ccccc4C(F)(F)F)CC3)c2C1 ; CC(C)(C)N1CCc2[nH]nc(C(=O)N3CCC(c4ccccc4C(F)(F)F)CC3)c2C1]

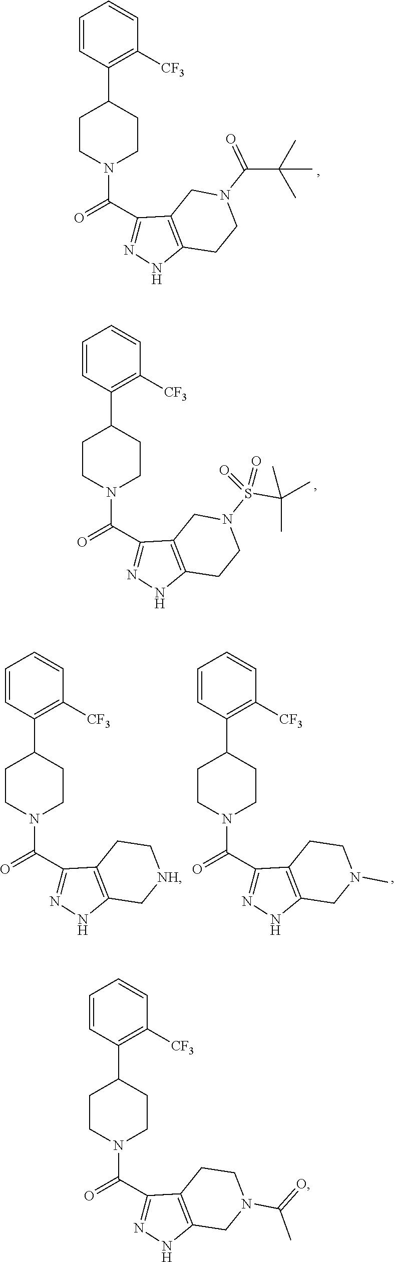

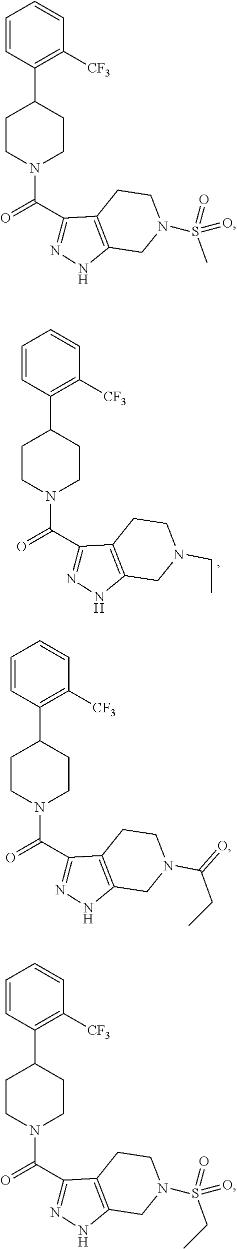 [SMILES: CS(=O)(=O)N1CCc2c(C(=O)N3CCC(c4ccccc4C(F)(F)F)CC3)n[nH]c2C1 ; CCN1CCc2c(C(=O)N3CCC(c4ccccc4C(F)(F)F)CC3)n[nH]c2C1 ; CCC(=O)N1CCc2c(C(=O)N3CCC(c4ccccc4C(F)(F)F)CC3)n[nH]c2C1 ; CCS(=O)(=O)N1CCc2c(C(=O)N3CCC(c4ccccc4C(F)(F)F)CC3)n[nH]c2C1]

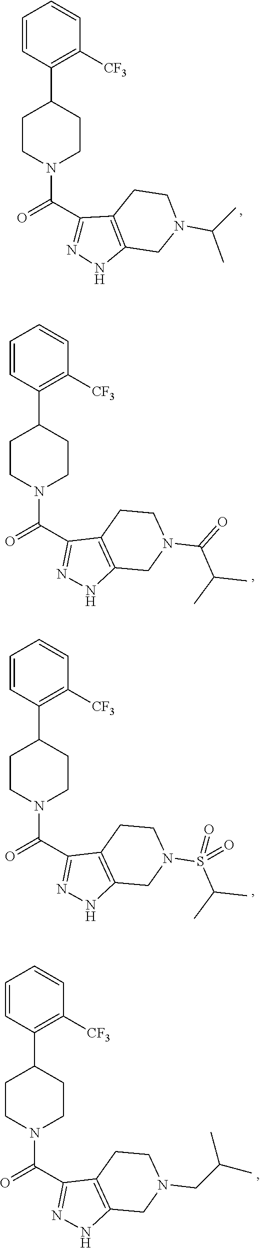 [SMILES: CC(C)N1CCc2c(C(=O)N3CCC(c4ccccc4C(F)(F)F)CC3)n[nH]c2C1 ; CC(C)C(=O)N1CCc2c(C(=O)N3CCC(c4ccccc4C(F)(F)F)CC3)n[nH]c2C1 ; CC(C)S(=O)(=O)N1CCc2c(C(=O)N3CCC(c4ccccc4C(F)(F)F)CC3)n[nH]c2C1 ; CC(C)CN1CCc2c(C(=O)N3CCC(c4ccccc4C(F)(F)F)CC3)n[nH]c2C1]

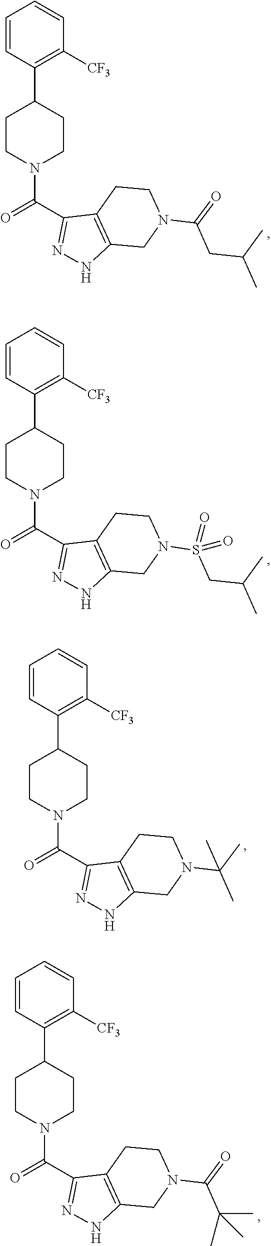 [SMILES: CC(C)CC(=O)N1CCc2c(C(=O)N3CCC(c4ccccc4C(F)(F)F)CC3)n[nH]c2C1 ; CC(C)CS(=O)(=O)N1CCc2c(C(=O)N3CCC(c4ccccc4C(F)(F)F)CC3)n[nH]c2C1 ; CC(C)(C)N1CCc2c(C(=O)N3CCC(c4ccccc4C(F)(F)F)CC3)n[nH]c2C1 ; CC(C)(C)C(=O)N1CCc2c(C(=O)N3CCC(c4ccccc4C(F)(F)F)CC3)n[nH]c2C1]

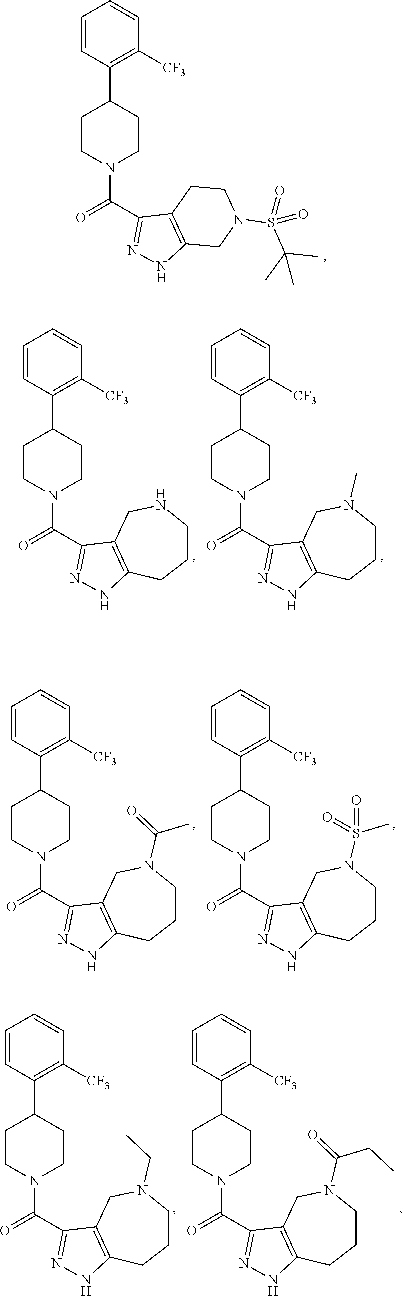

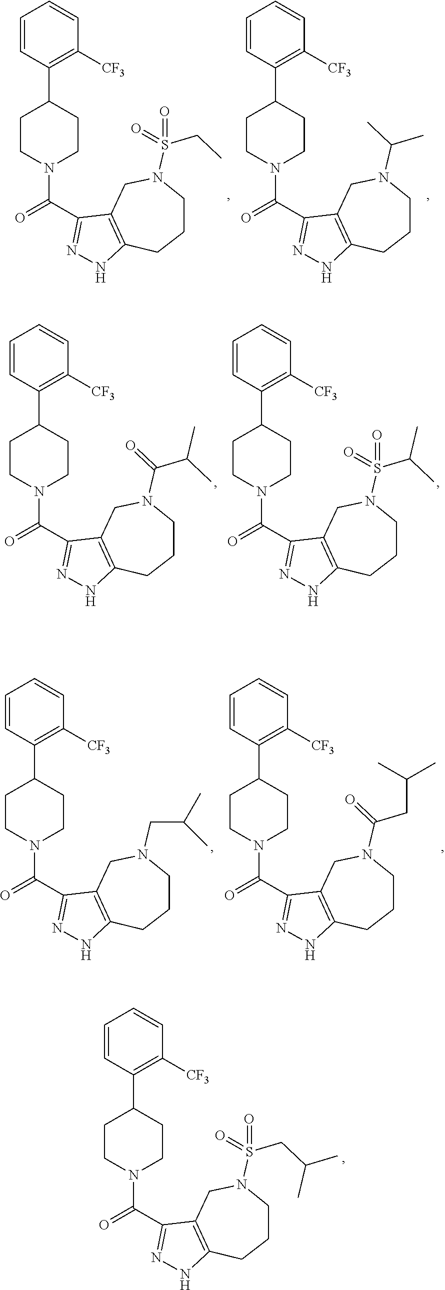

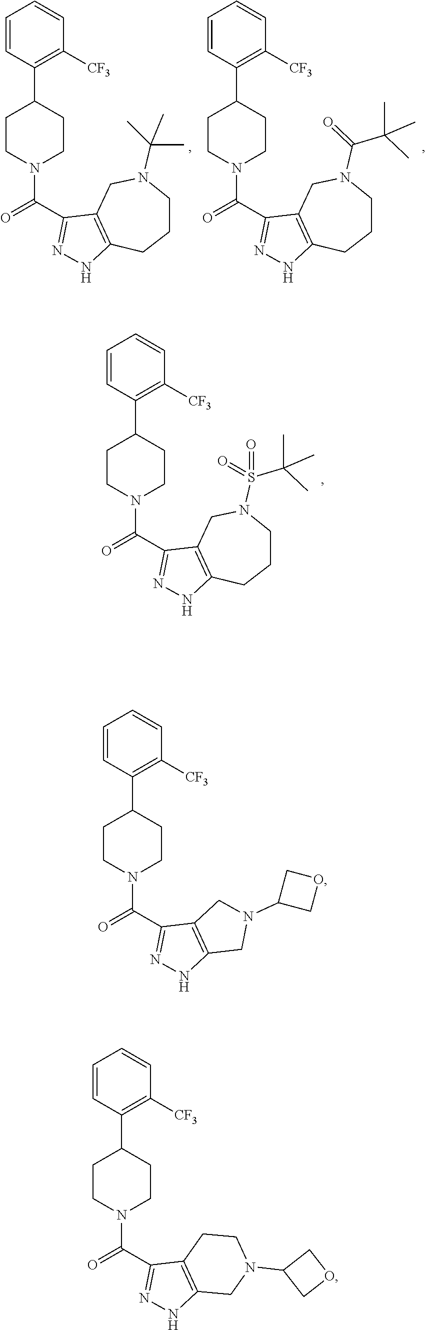

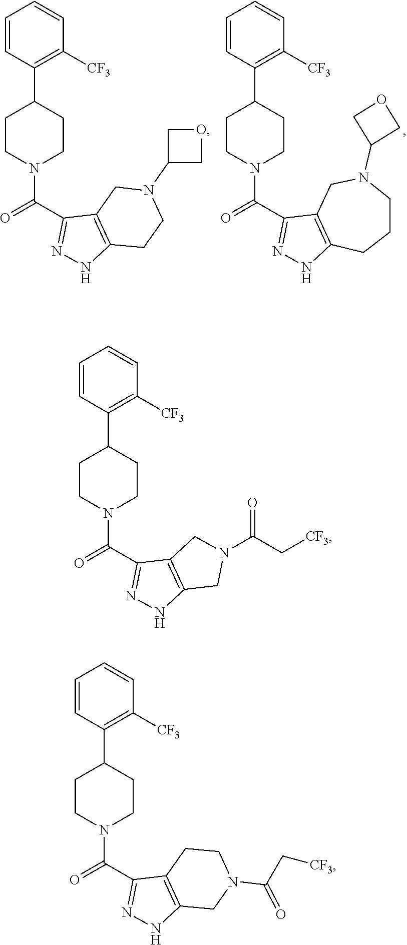

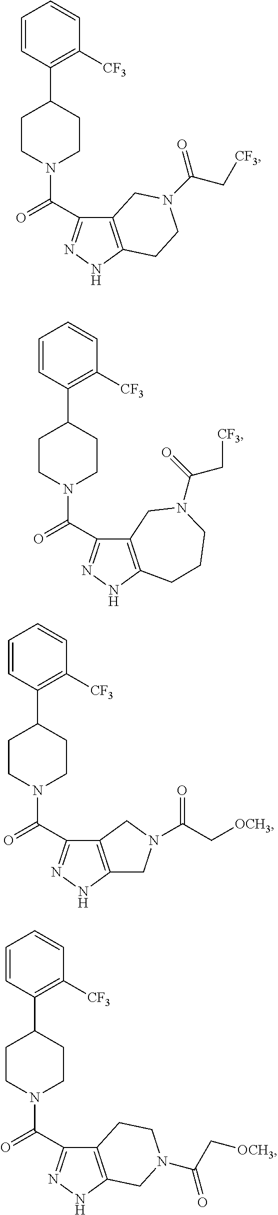 [SMILES: O=C(CC(F)(F)F)N1CCc2[nH]nc(C(=O)N3CCC(c4ccccc4C(F)(F)F)CC3)c2C1 ; O=C(CC(F)(F)F)N1CCCc2[nH]nc(C(=O)N3CCC(c4ccccc4C(F)(F)F)CC3)c2C1 ; COCC(=O)N1Cc2[nH]nc(C(=O)N3CCC(c4ccccc4C(F)(F)F)CC3)c2C1 ; COCC(=O)N1CCc2c(C(=O)N3CCC(c4ccccc4C(F)(F)F)CC3)n[nH]c2C1]

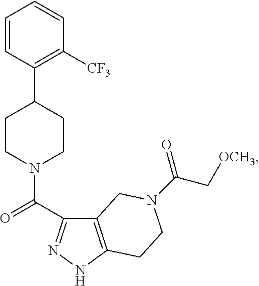 [SMILES: COCC(=O)N1CCc2[nH]nc(C(=O)N3CCC(c4ccccc4C(F)(F)F)CC3)c2C1]

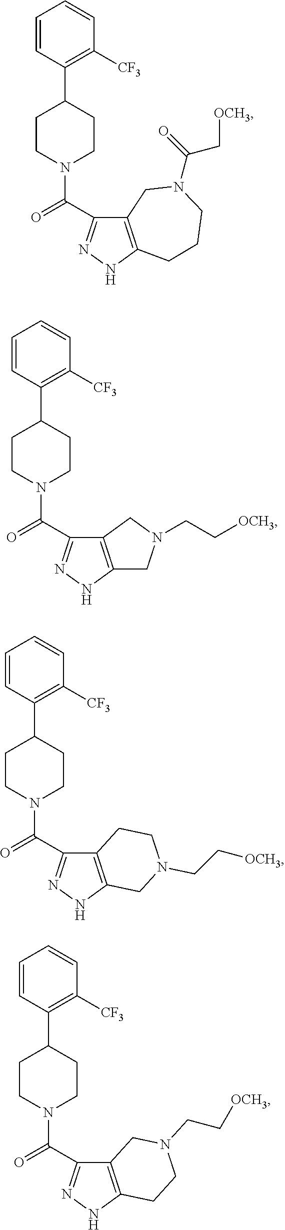 [SMILES: COCC(=O)N1CCCc2[nH]nc(C(=O)N3CCC(c4ccccc4C(F)(F)F)CC3)c2C1 ; COCCN1Cc2[nH]nc(C(=O)N3CCC(c4ccccc4C(F)(F)F)CC3)c2C1 ; COCCN1CCc2c(C(=O)N3CCC(c4ccccc4C(F)(F)F)CC3)n[nH]c2C1 ; COCCN1CCc2[nH]nc(C(=O)N3CCC(c4ccccc4C(F)(F)F)CC3)c2C1]

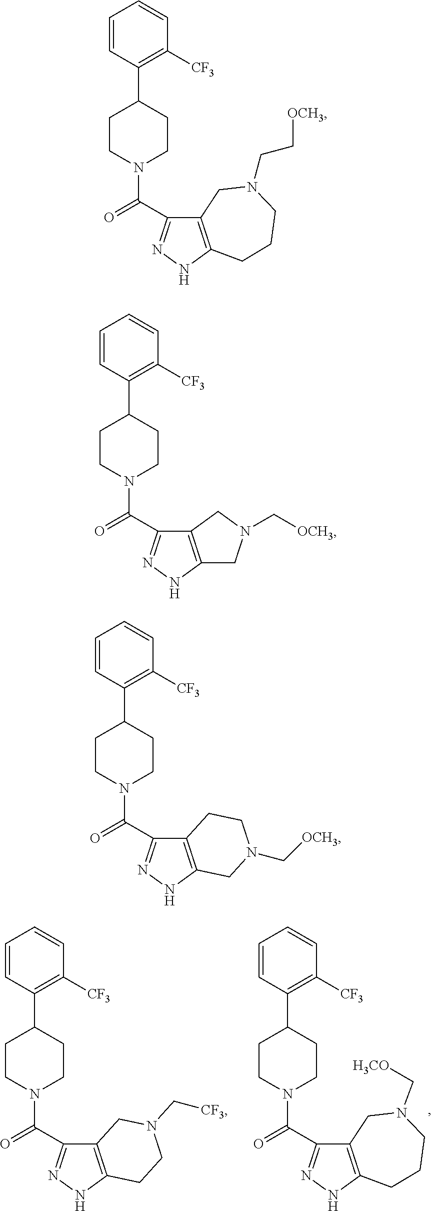 [SMILES: COCCN1CCCc2[nH]nc(C(=O)N3CCC(c4ccccc4C(F)(F)F)CC3)c2C1 ; COCN1Cc2[nH]nc(C(=O)N3CCC(c4ccccc4C(F)(F)F)CC3)c2C1 ; COCN1CCc2c(C(=O)N3CCC(c4ccccc4C(F)(F)F)CC3)n[nH]c2C1 ; O=C(c1n[nH]c2c1CN(CC(F)(F)F)CC2)N1CCC(c2ccccc2C(F)(F)F)CC1 ; COCN1CCCc2[nH]nc(C(=O)N3CCC(c4ccccc4C(F)(F)F)CC3)c2C1]

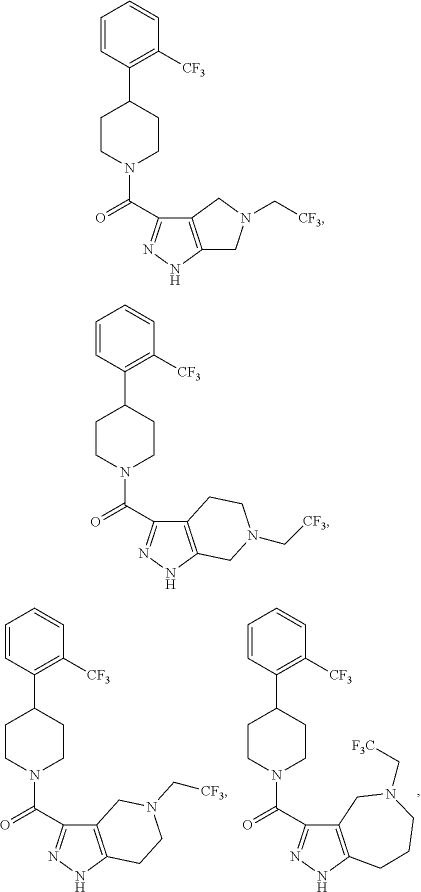 [SMILES: O=C(c1n[nH]c2c1CN(CC(F)(F)F)C2)N1CCC(c2ccccc2C(F)(F)F)CC1 ; O=C(c1n[nH]c2c1CCN(CC(F)(F)F)C2)N1CCC(c2ccccc2C(F)(F)F)CC1 ; O=C(c1n[nH]c2c1CN(CC(F)(F)F)CC2)N1CCC(c2ccccc2C(F)(F)F)CC1 ; O=C(c1n[nH]c2c1CN(CC(F)(F)F)CCC2)N1CCC(c2ccccc2C(F)(F)F)CC1]

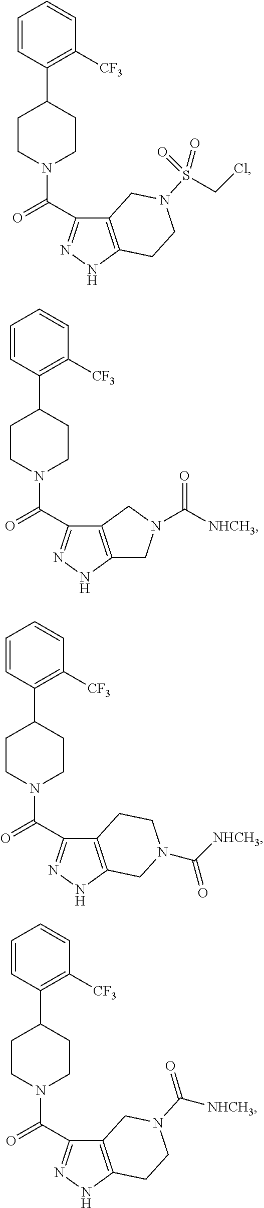 [SMILES: O=C(c1n[nH]c2c1CN(S(=O)(=O)CCl)CC2)N1CCC(c2ccccc2C(F)(F)F)CC1 ; CNC(=O)N1Cc2[nH]nc(C(=O)N3CCC(c4ccccc4C(F)(F)F)CC3)c2C1 ; CNC(=O)N1CCc2c(C(=O)N3CCC(c4ccccc4C(F)(F)F)CC3)n[nH]c2C1 ; CNC(=O)N1CCc2[nH]nc(C(=O)N3CCC(c4ccccc4C(F)(F)F)CC3)c2C1]

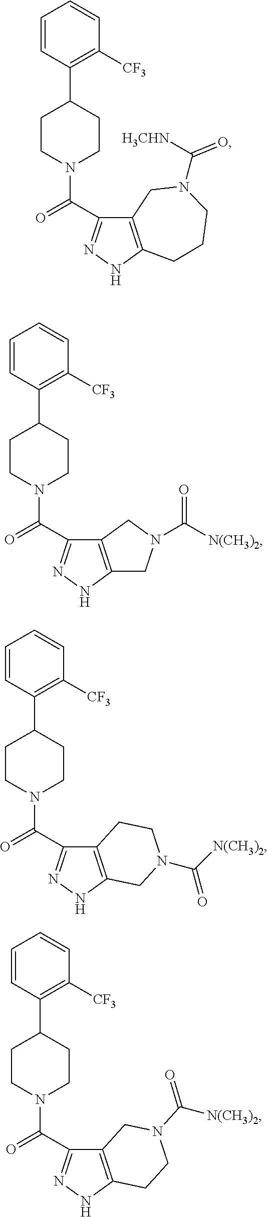

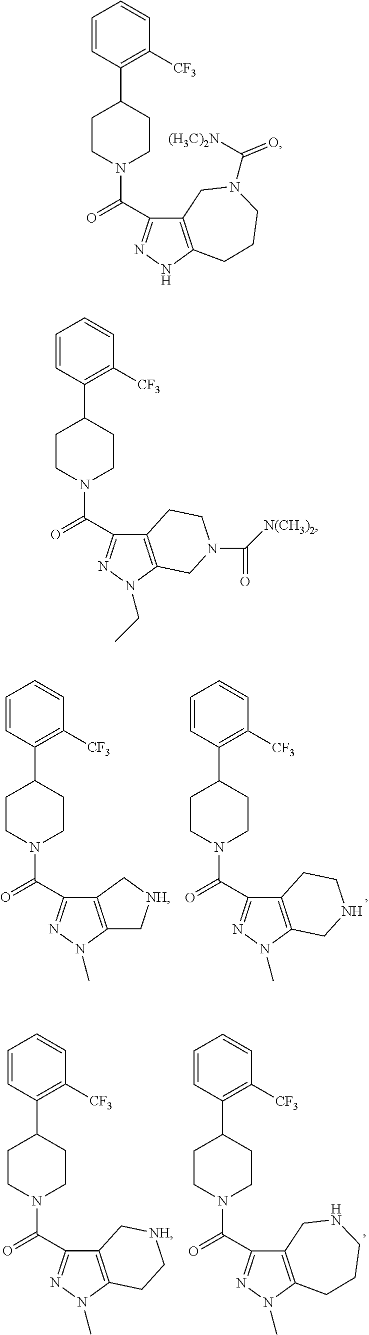

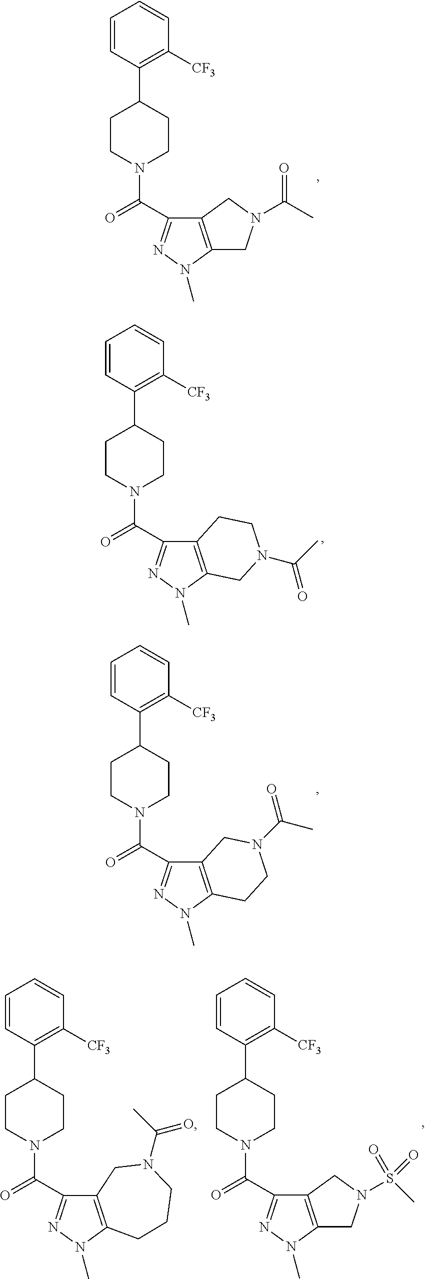

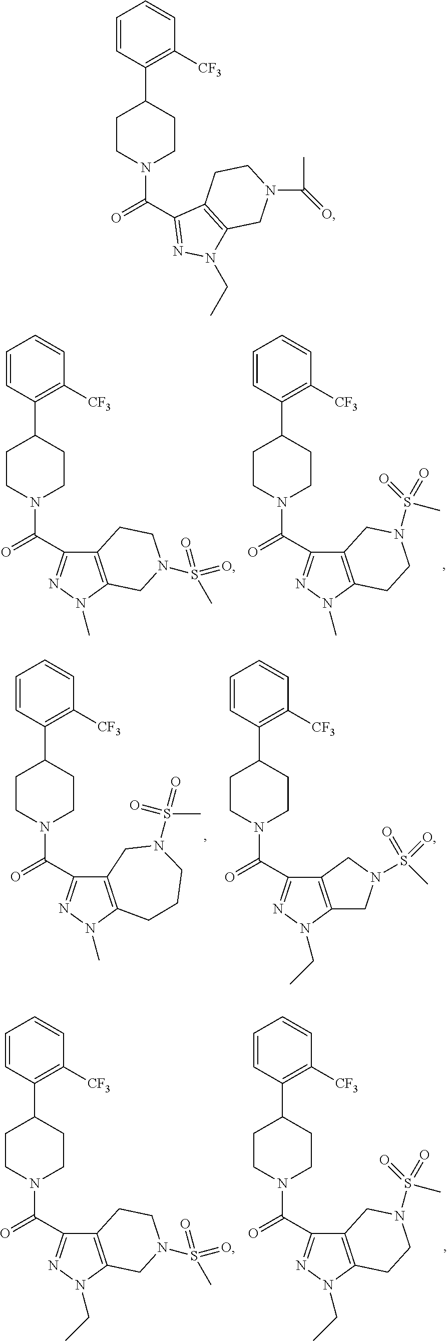

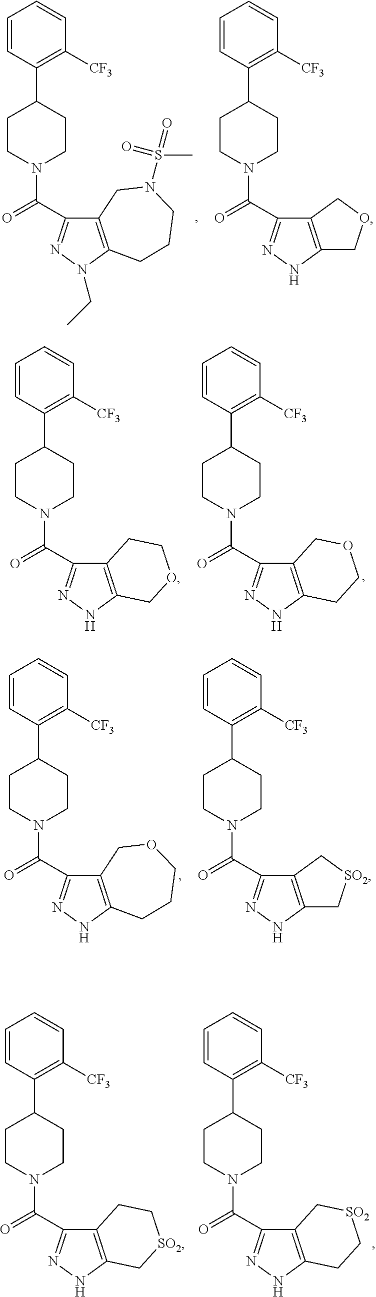

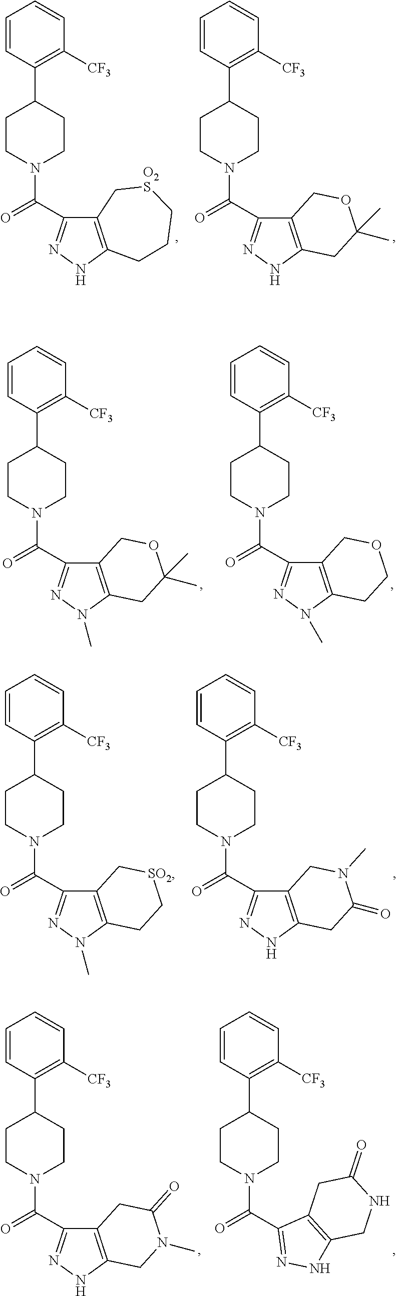

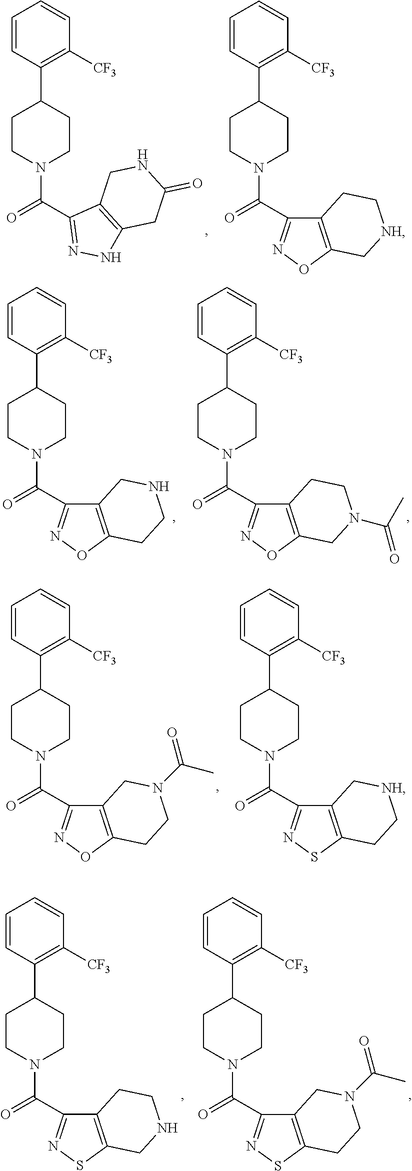

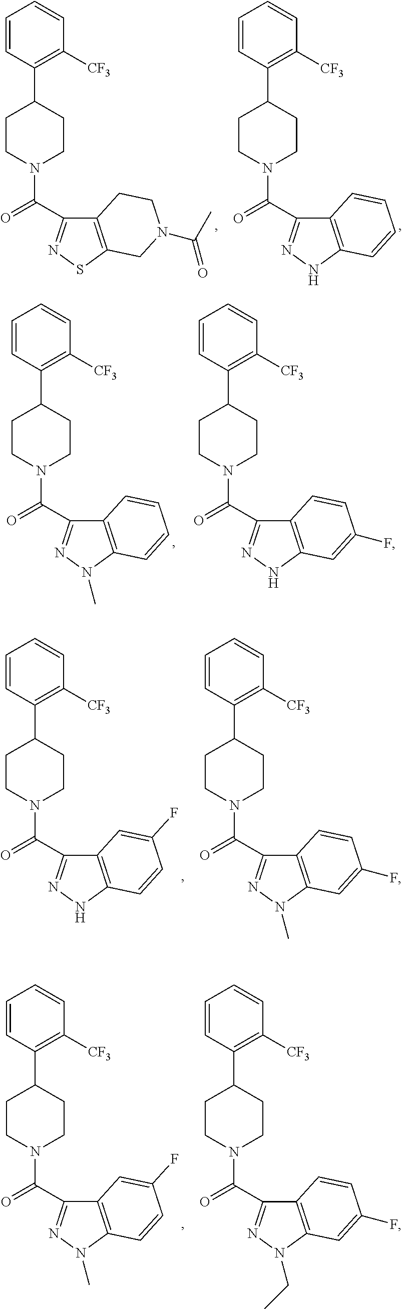

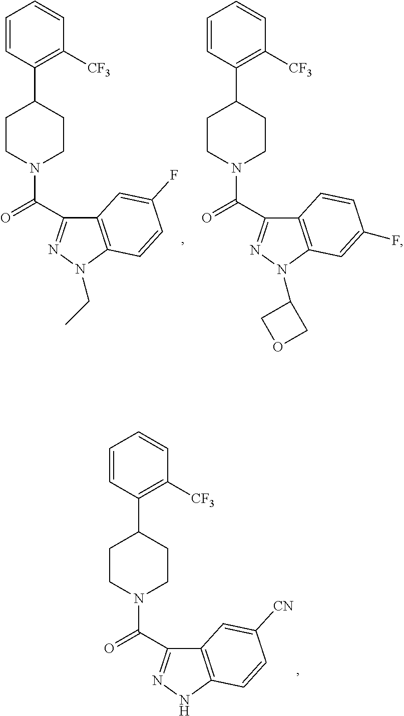

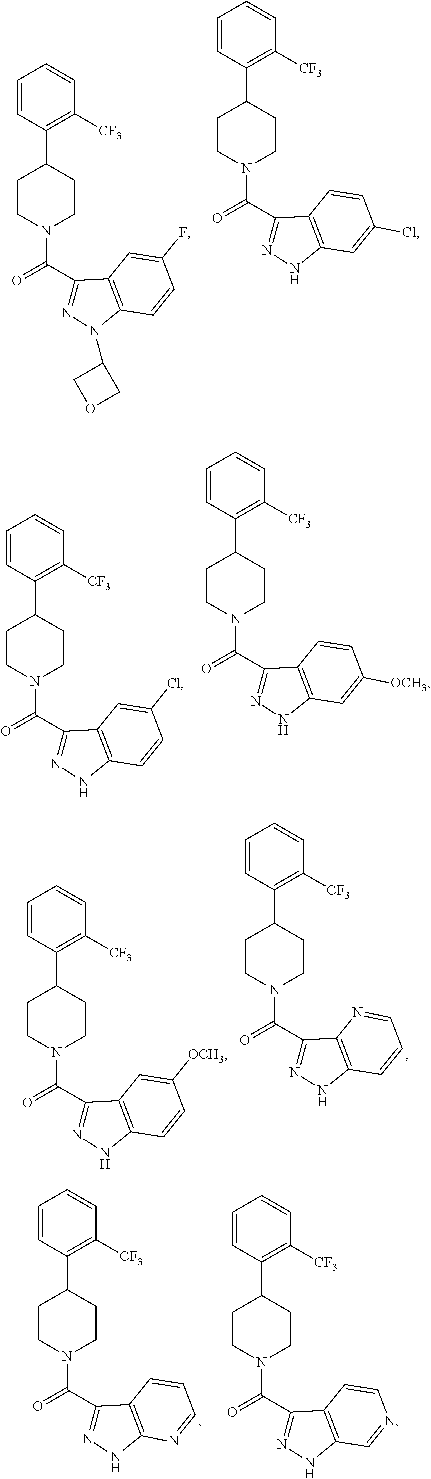

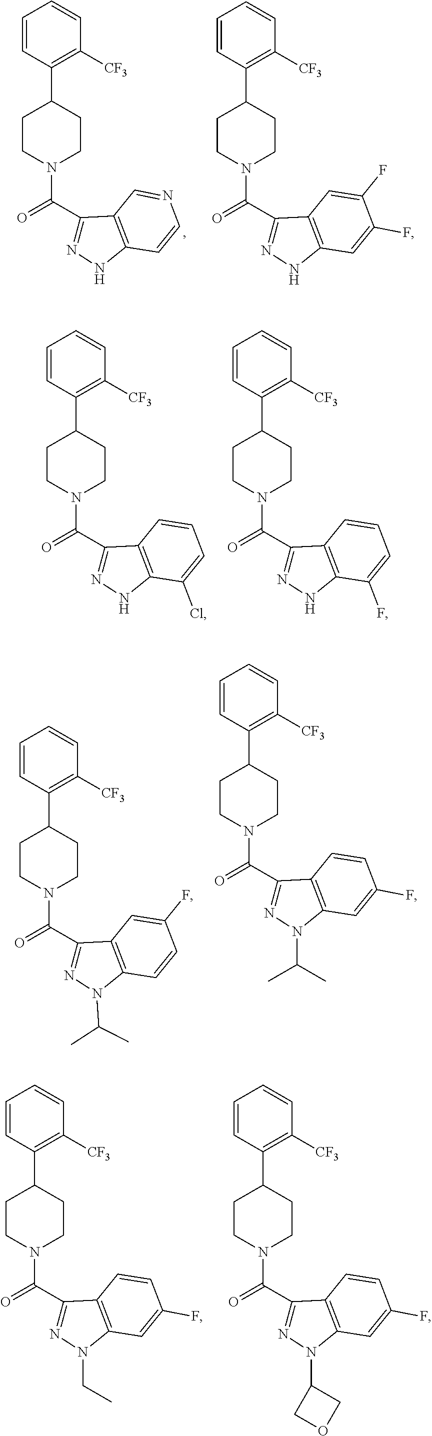

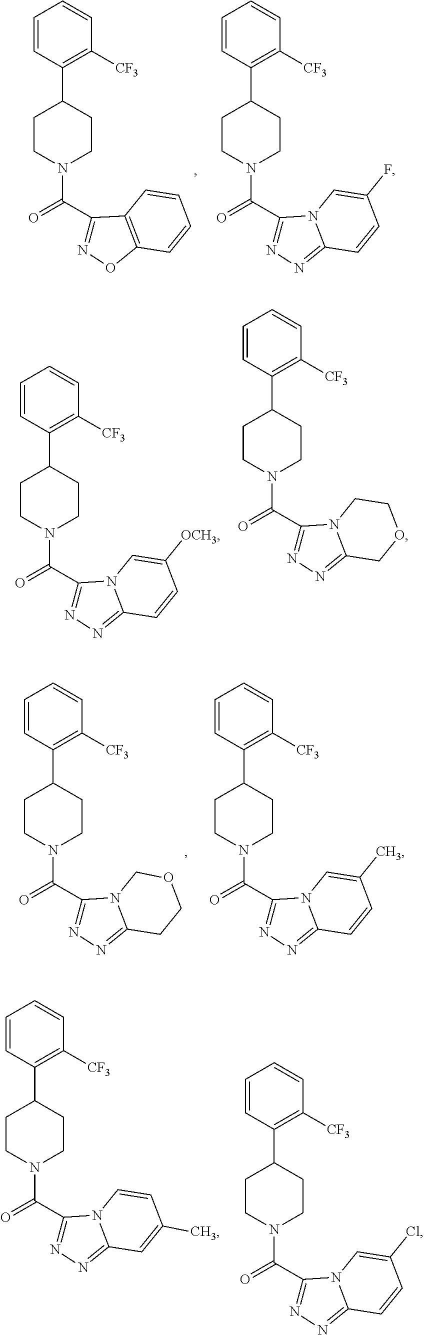

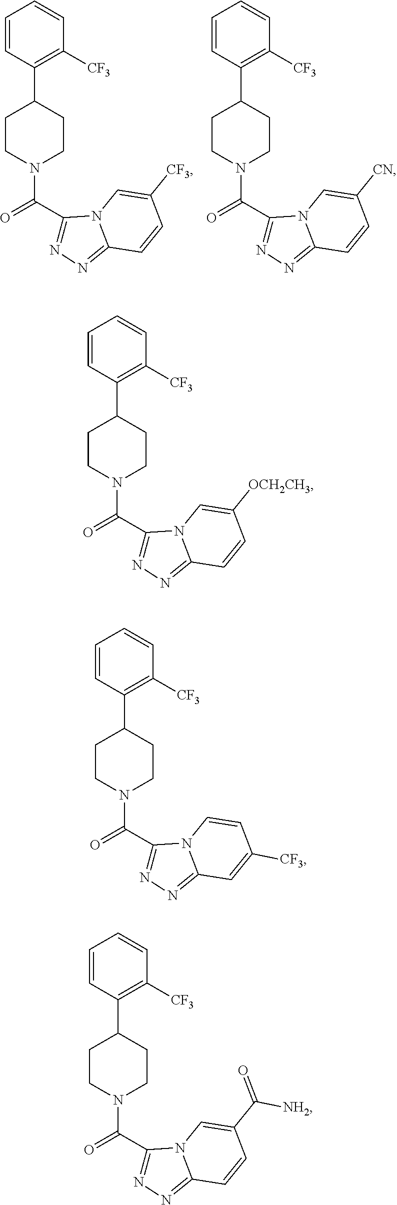

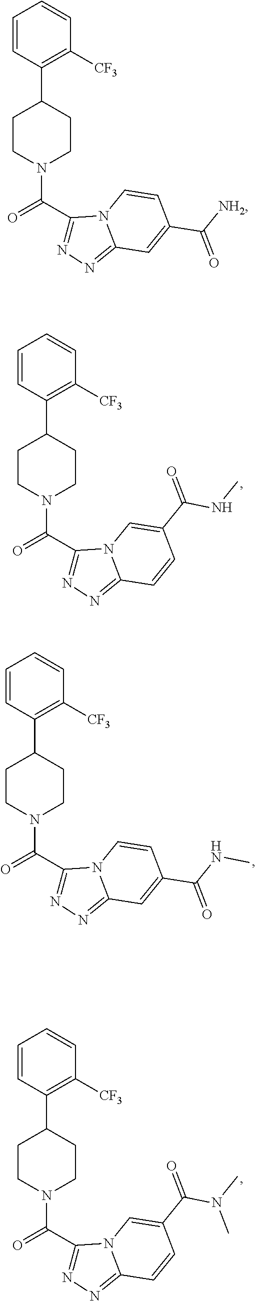 [SMILES: NC(=O)c1ccn2c(C(=O)N3CCC(c4ccccc4C(F)(F)F)CC3)nnc2c1 ; CNC(=O)c1ccc2nnc(C(=O)N3CCC(c4ccccc4C(F)(F)F)CC3)n2c1 ; CNC(=O)c1ccn2c(C(=O)N3CCC(c4ccccc4C(F)(F)F)CC3)nnc2c1 ; CN(C)C(=O)c1ccc2nnc(C(=O)N3CCC(c4ccccc4C(F)(F)F)CC3)n2c1]

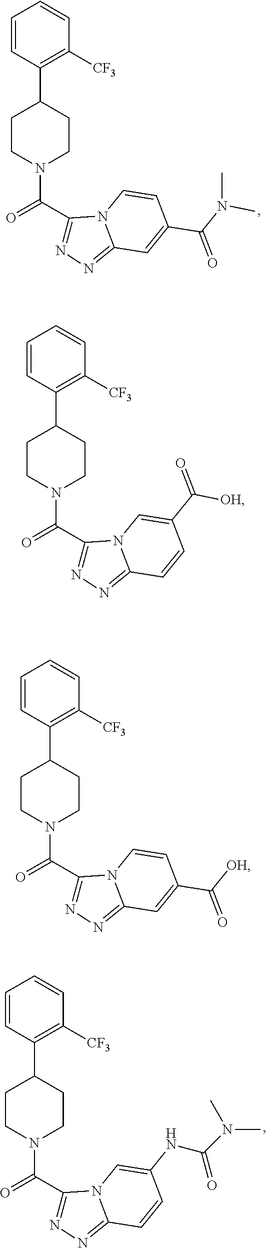 [SMILES: CN(C)C(=O)c1ccn2c(C(=O)N3CCC(c4ccccc4C(F)(F)F)CC3)nnc2c1 ; O=C(O)c1ccc2nnc(C(=O)N3CCC(c4ccccc4C(F)(F)F)CC3)n2c1 ; O=C(O)c1ccn2c(C(=O)N3CCC(c4ccccc4C(F)(F)F)CC3)nnc2c1 ; CN(C)C(=O)Nc1ccc2nnc(C(=O)N3CCC(c4ccccc4C(F)(F)F)CC3)n2c1]

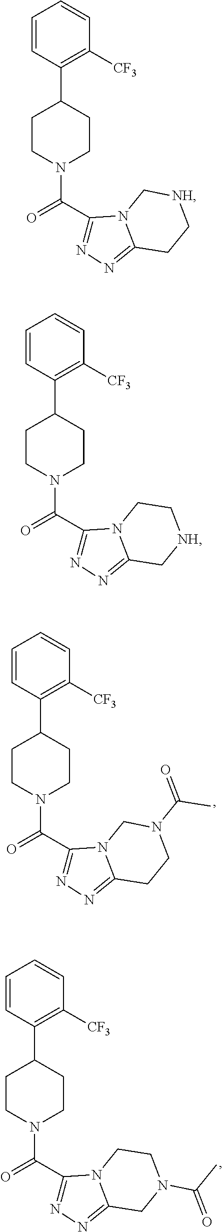 [SMILES: O=C(c1nnc2n1CNCC2)N1CCC(c2ccccc2C(F)(F)F)CC1 ; O=C(c1nnc2n1CCNC2)N1CCC(c2ccccc2C(F)(F)F)CC1 ; CC(=O)N1CCc2nnc(C(=O)N3CCC(c4ccccc4C(F)(F)F)CC3)n2C1 ; CC(=O)N1CCn2c(nnc2C(=O)N2CCC(c3ccccc3C(F)(F)F)CC2)C1]

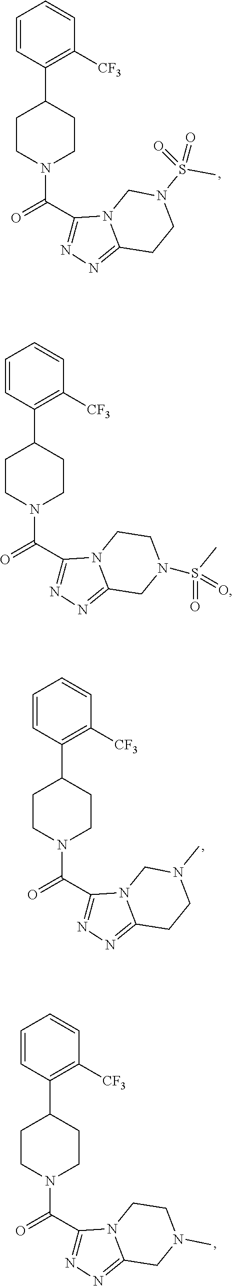 [SMILES: CS(=O)(=O)N1CCc2nnc(C(=O)N3CCC(c4ccccc4C(F)(F)F)CC3)n2C1 ; CS(=O)(=O)N1CCn2c(nnc2C(=O)N2CCC(c3ccccc3C(F)(F)F)CC2)C1 ; CN1CCc2nnc(C(=O)N3CCC(c4ccccc4C(F)(F)F)CC3)n2C1 ; CN1CCn2c(nnc2C(=O)N2CCC(c3ccccc3C(F)(F)F)CC2)C1]

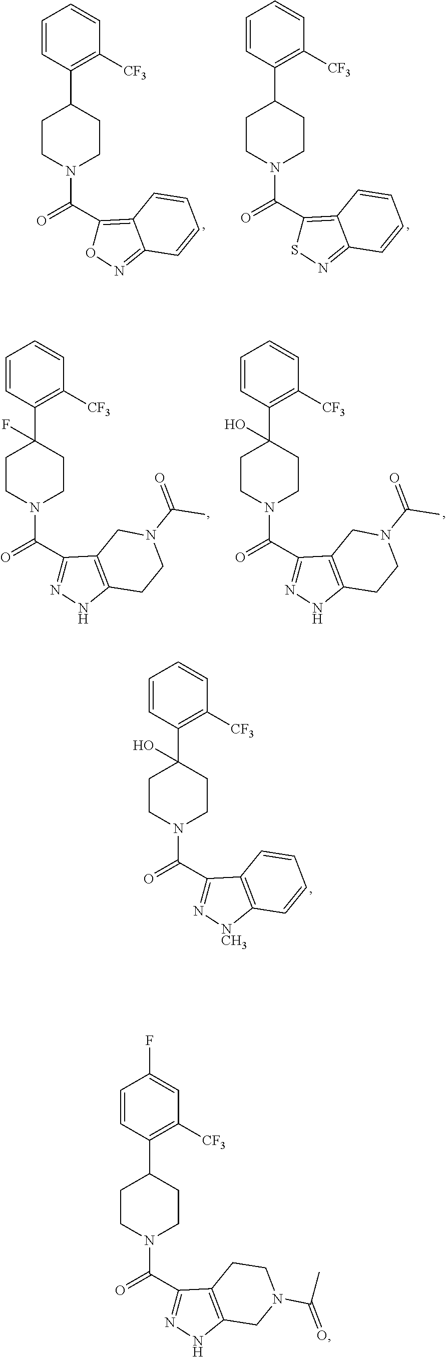

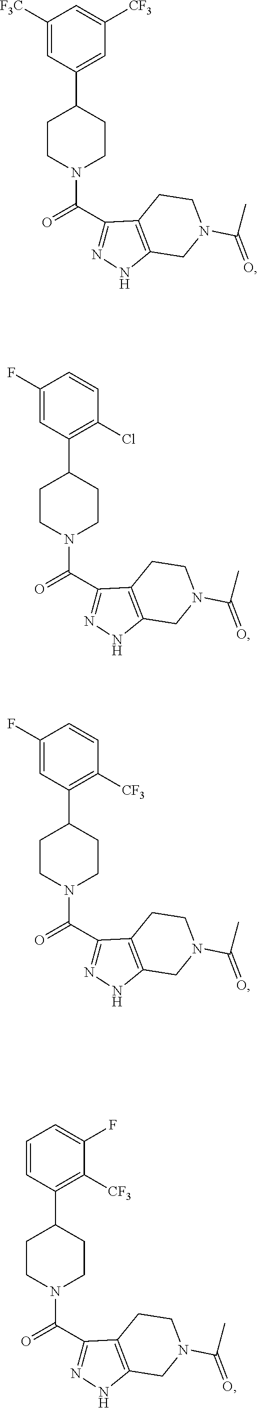 [SMILES: CC(=O)N1CCc2c(C(=O)N3CCC(c4cc(C(F)(F)F)cc(C(F)(F)F)c4)CC3)n[nH]c2C1 ; CC(=O)N1CCc2c(C(=O)N3CCC(c4cc(F)ccc4Cl)CC3)n[nH]c2C1 ; CC(=O)N1CCc2c(C(=O)N3CCC(c4cc(F)ccc4C(F)(F)F)CC3)n[nH]c2C1 ; CC(=O)N1CCc2c(C(=O)N3CCC(c4cccc(F)c4C(F)(F)F)CC3)n[nH]c2C1]

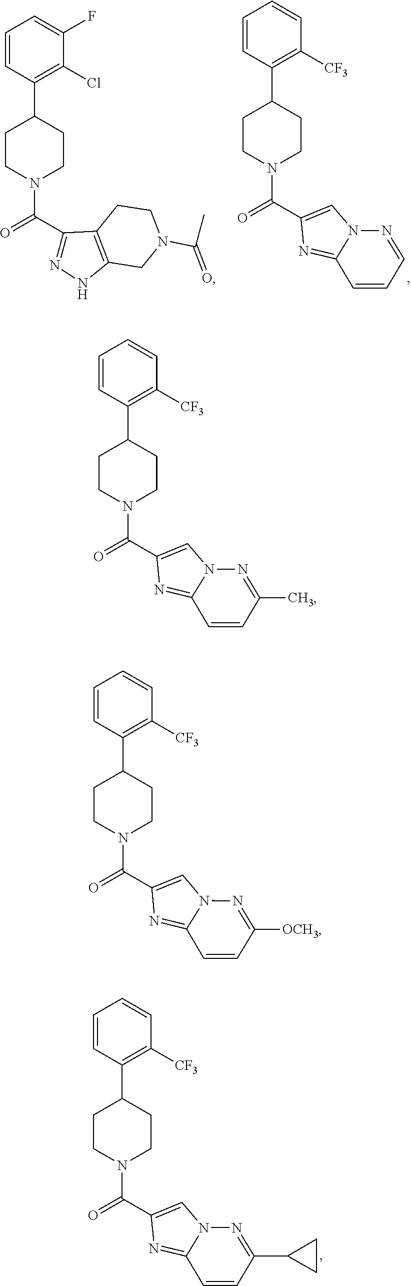

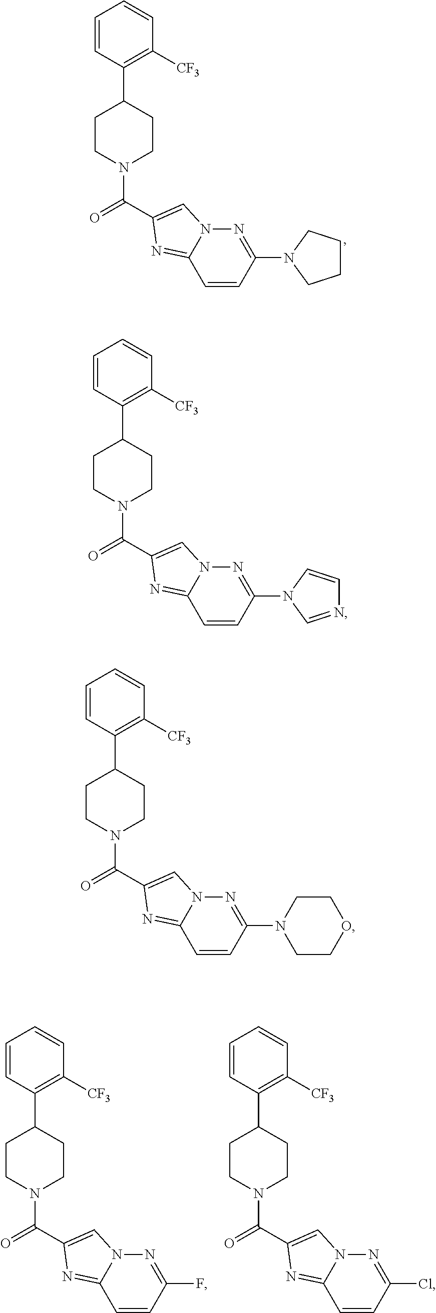 [SMILES: O=C(c1cn2nc(N3CCCC3)ccc2n1)N1CCC(c2ccccc2C(F)(F)F)CC1 ; O=C(c1cn2nc(-n3ccnc3)ccc2n1)N1CCC(c2ccccc2C(F)(F)F)CC1 ; O=C(c1cn2nc(N3CCOCC3)ccc2n1)N1CCC(c2ccccc2C(F)(F)F)CC1 ; O=C(c1cn2nc(F)ccc2n1)N1CCC(c2ccccc2C(F)(F)F)CC1 ; O=C(c1cn2nc(Cl)ccc2n1)N1CCC(c2ccccc2C(F)(F)F)CC1]

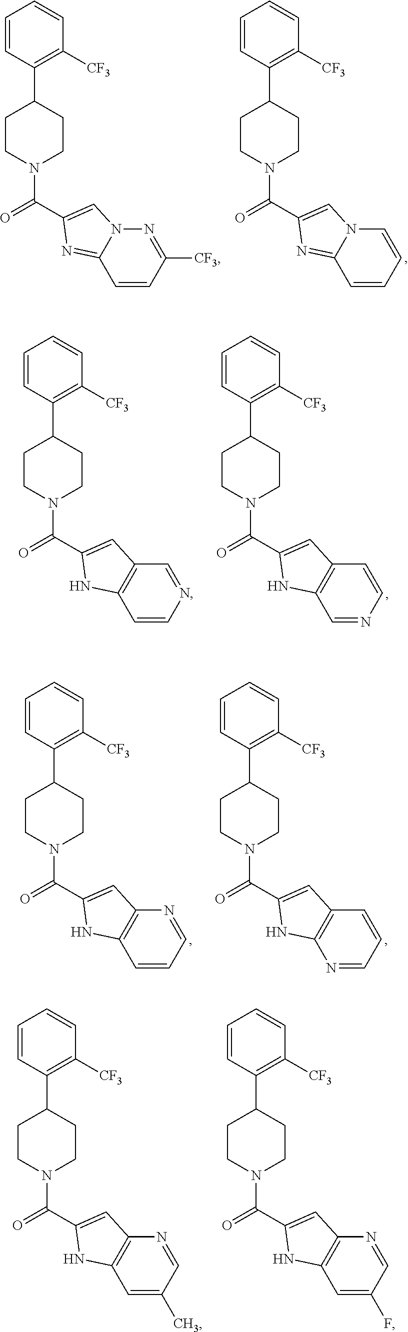

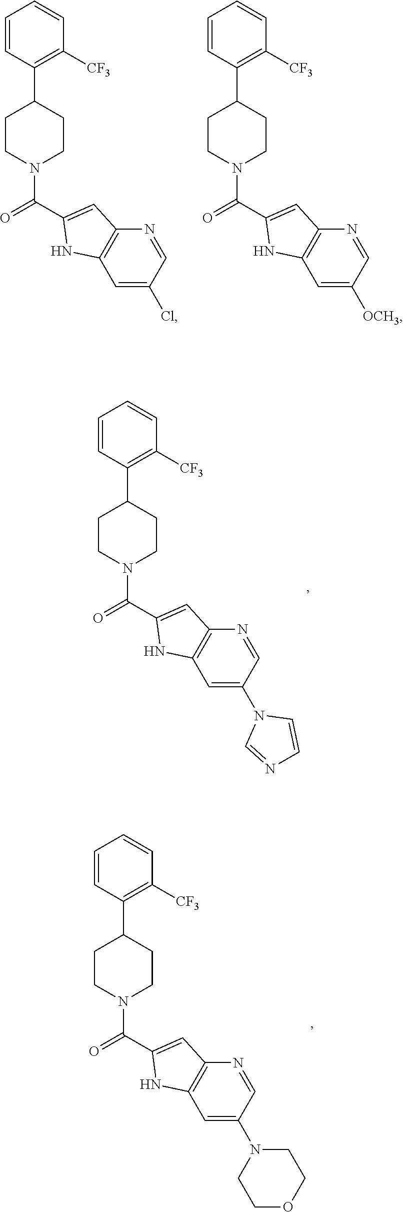 [SMILES: O=C(c1cc2ncc(Cl)cc2[nH]1)N1CCC(c2ccccc2C(F)(F)F)CC1 ; COc1cnc2cc(C(=O)N3CCC(c4ccccc4C(F)(F)F)CC3)[nH]c2c1 ; O=C(c1cc2ncc(-n3ccnc3)cc2[nH]1)N1CCC(c2ccccc2C(F)(F)F)CC1 ; O=C(c1cc2ncc(N3CCOCC3)cc2[nH]1)N1CCC(c2ccccc2C(F)(F)F)CC1]

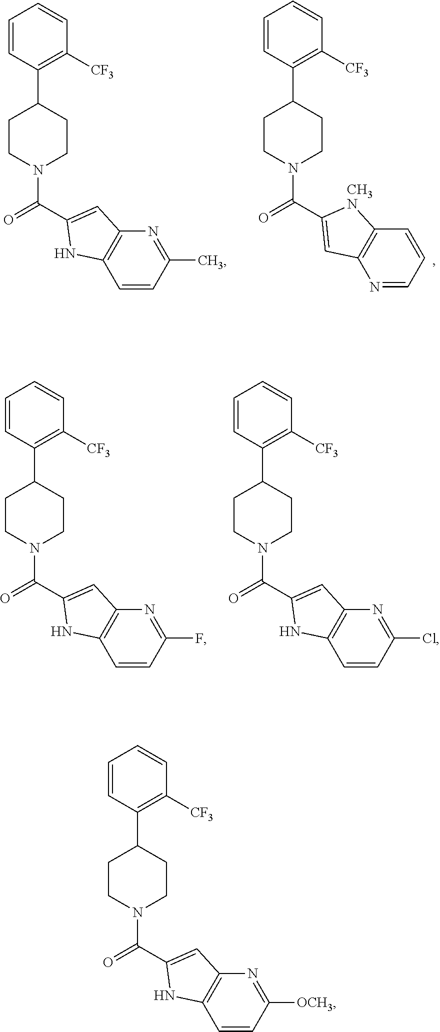

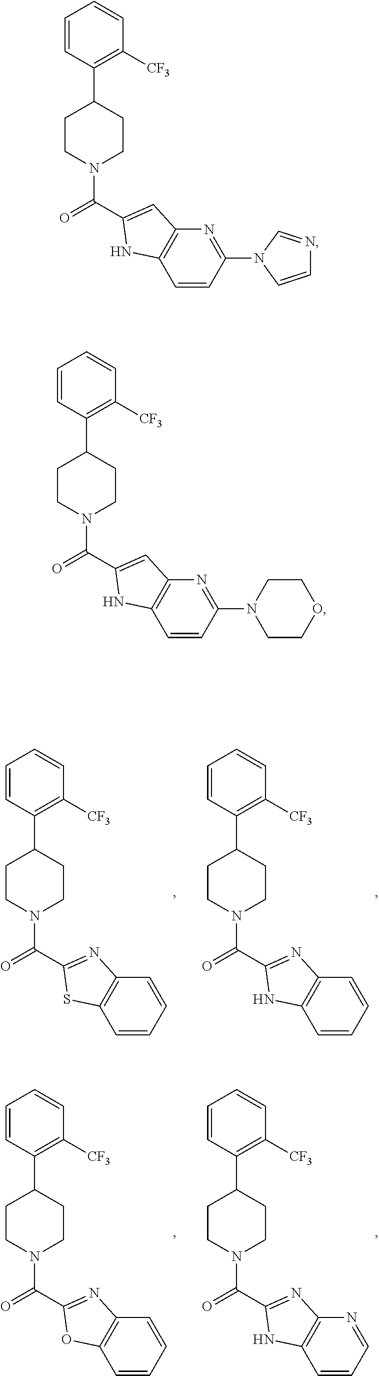 [SMILES: O=C(c1cc2nc(-n3ccnc3)ccc2[nH]1)N1CCC(c2ccccc2C(F)(F)F)CC1 ; O=C(c1cc2nc(N3CCOCC3)ccc2[nH]1)N1CCC(c2ccccc2C(F)(F)F)CC1 ; O=C(c1nc2ccccc2s1)N1CCC(c2ccccc2C(F)(F)F)CC1 ; O=C(c1nc2ccccc2[nH]1)N1CCC(c2ccccc2C(F)(F)F)CC1 ; O=C(c1nc2ccccc2o1)N1CCC(c2ccccc2C(F)(F)F)CC1 ; O=C(c1nc2ncccc2[nH]1)N1CCC(c2ccccc2C(F)(F)F)CC1]

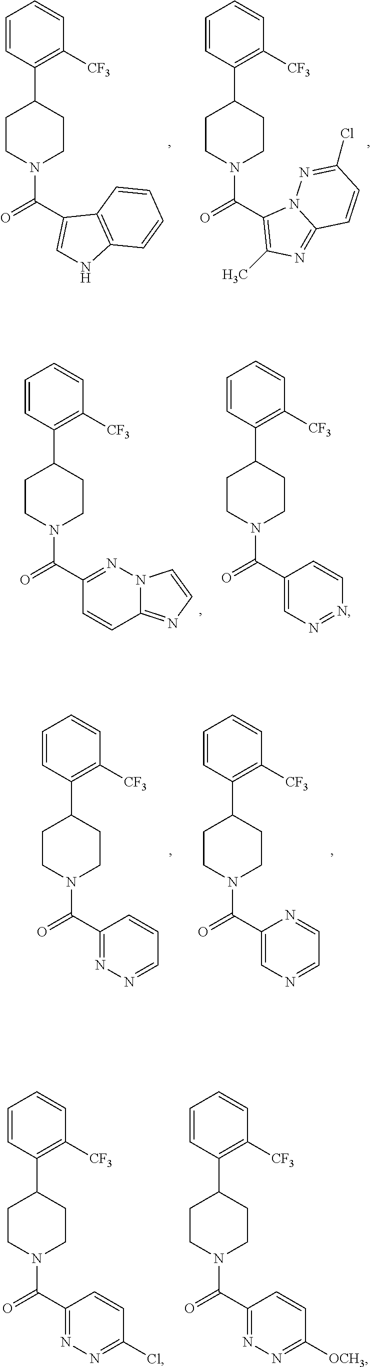

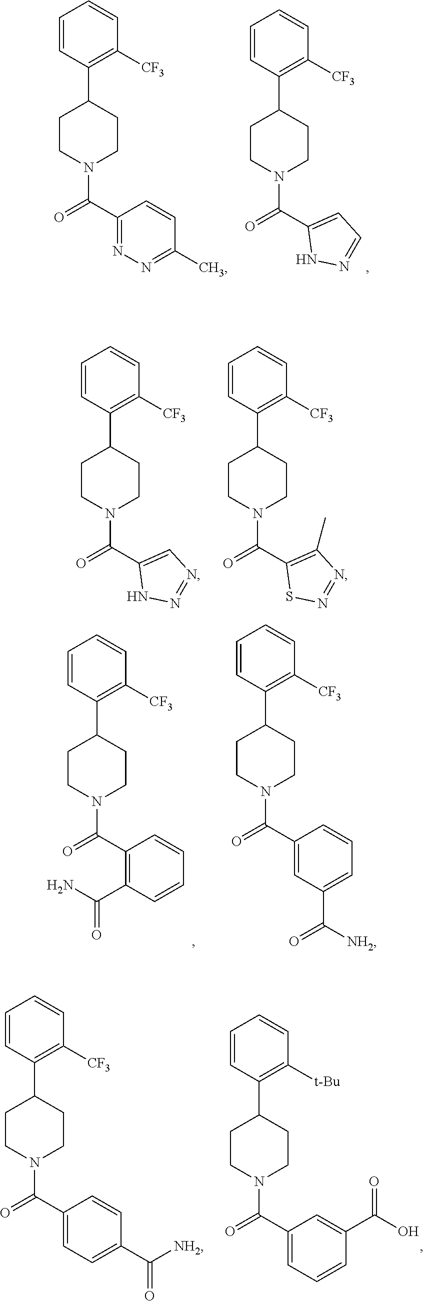

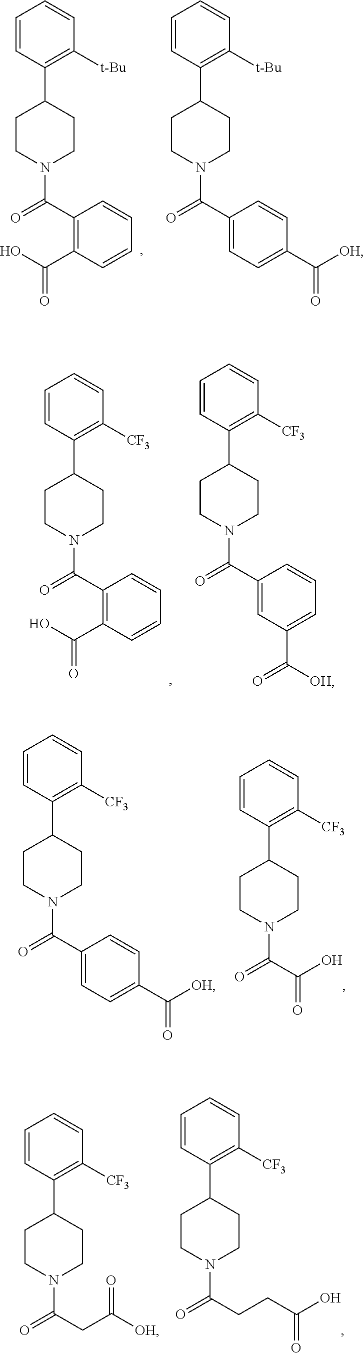

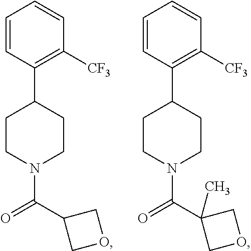

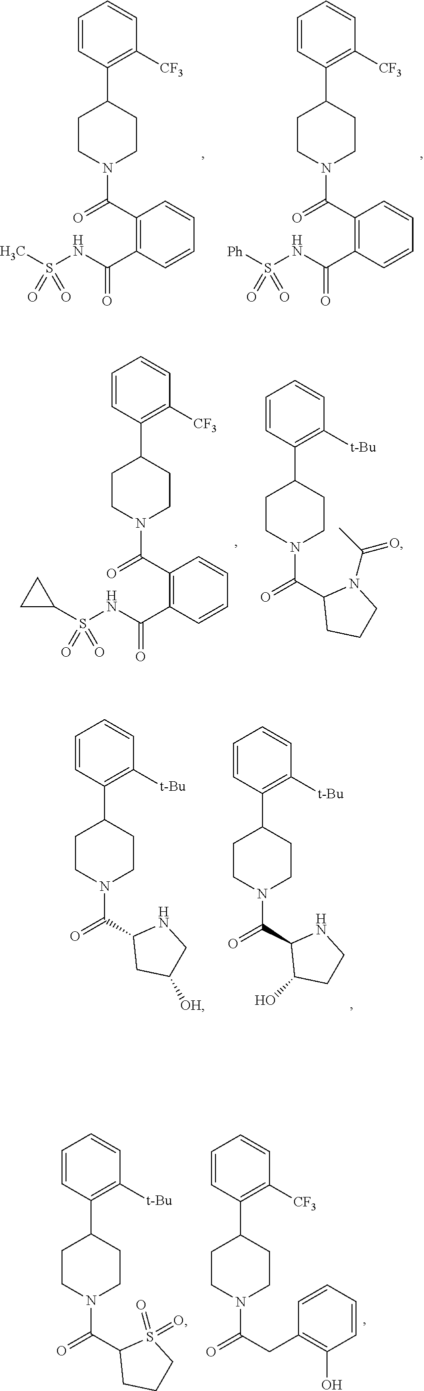

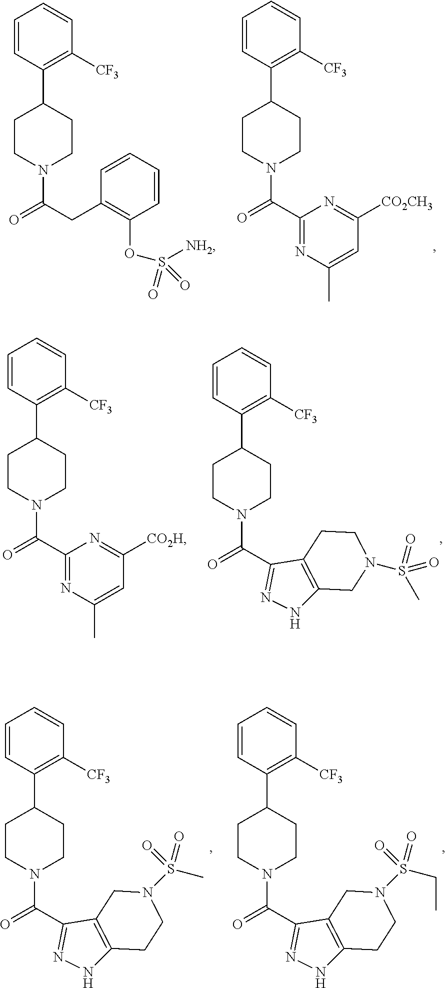

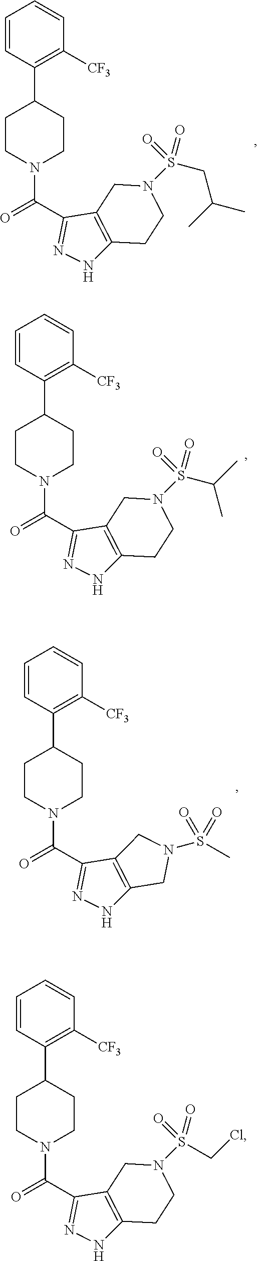 [SMILES: CC(C)CS(=O)(=O)N1CCc2[nH]nc(C(=O)N3CCC(c4ccccc4C(F)(F)F)CC3)c2C1 ; CC(C)S(=O)(=O)N1CCc2[nH]nc(C(=O)N3CCC(c4ccccc4C(F)(F)F)CC3)c2C1 ; CS(=O)(=O)N1Cc2[nH]nc(C(=O)N3CCC(c4ccccc4C(F)(F)F)CC3)c2C1 ; O=C(c1n[nH]c2c1CN(S(=O)(=O)CCl)CC2)N1CCC(c2ccccc2C(F)(F)F)CC1]

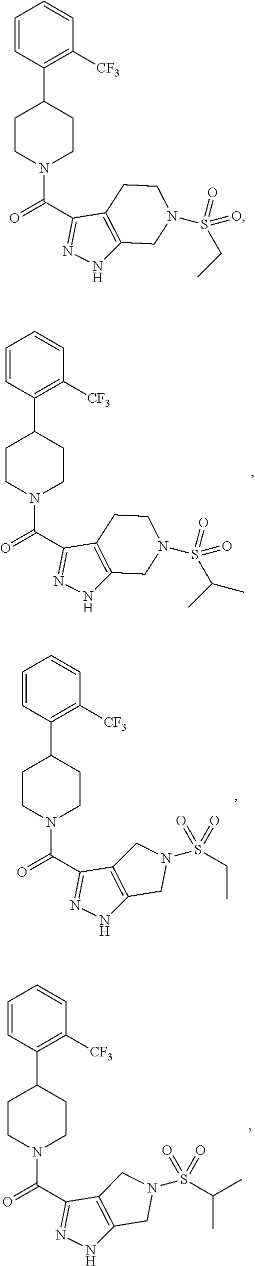 [SMILES: CCS(=O)(=O)N1CCc2c(C(=O)N3CCC(c4ccccc4C(F)(F)F)CC3)n[nH]c2C1 ; CC(C)S(=O)(=O)N1CCc2c(C(=O)N3CCC(c4ccccc4C(F)(F)F)CC3)n[nH]c2C1 ; CCS(=O)(=O)N1Cc2[nH]nc(C(=O)N3CCC(c4ccccc4C(F)(F)F)CC3)c2C1 ; CC(C)S(=O)(=O)N1Cc2[nH]nc(C(=O)N3CCC(c4ccccc4C(F)(F)F)CC3)c2C1]

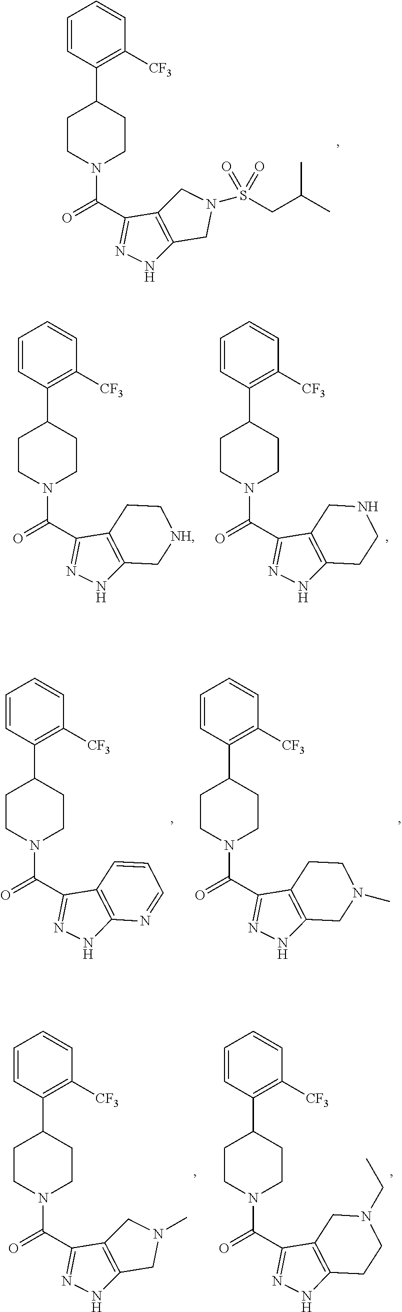

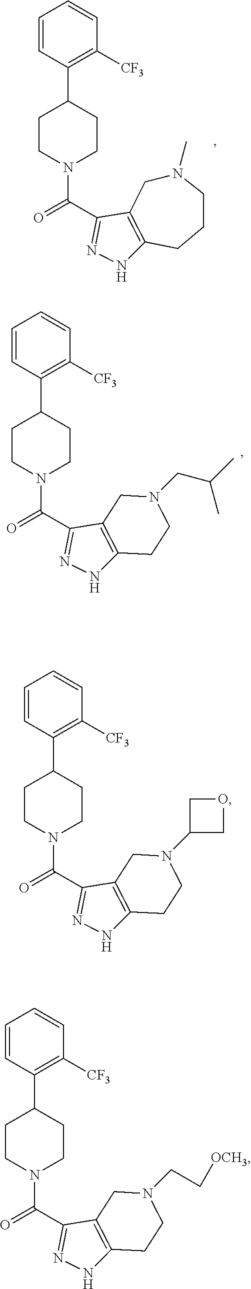 [SMILES: CN1CCCc2[nH]nc(C(=O)N3CCC(c4ccccc4C(F)(F)F)CC3)c2C1 ; CC(C)CN1CCc2[nH]nc(C(=O)N3CCC(c4ccccc4C(F)(F)F)CC3)c2C1 ; O=C(c1n[nH]c2c1CN(C1COC1)CC2)N1CCC(c2ccccc2C(F)(F)F)CC1 ; COCCN1CCc2[nH]nc(C(=O)N3CCC(c4ccccc4C(F)(F)F)CC3)c2C1]

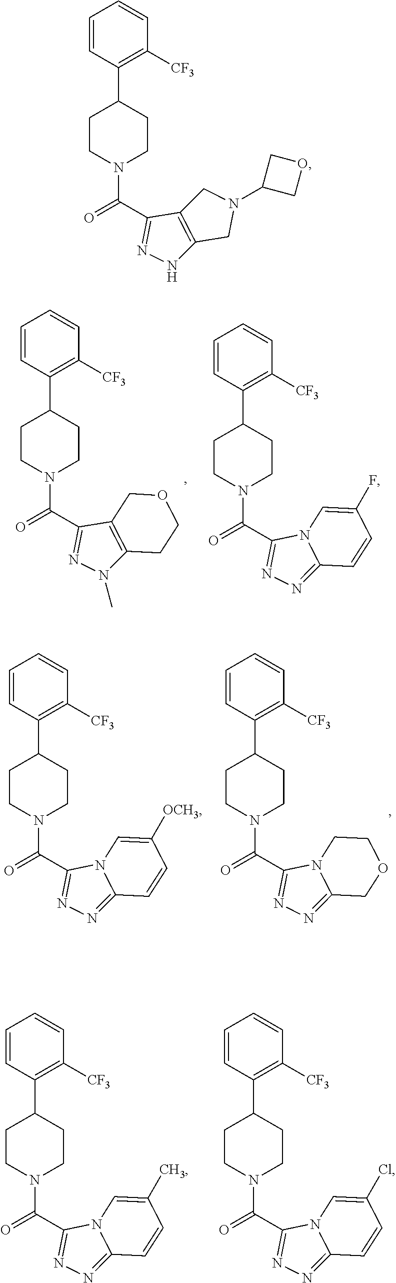

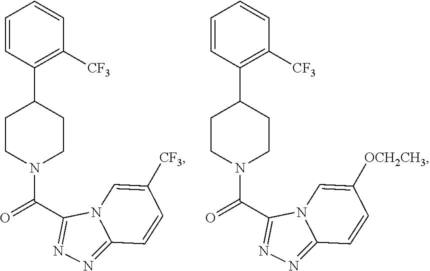 [SMILES: O=C(c1nnc2ccc(C(F)(F)F)cn12)N1CCC(c2ccccc2C(F)(F)F)CC1 ; CCOc1ccc2nnc(C(=O)N3CCC(c4ccccc4C(F)(F)F)CC3)n2c1]

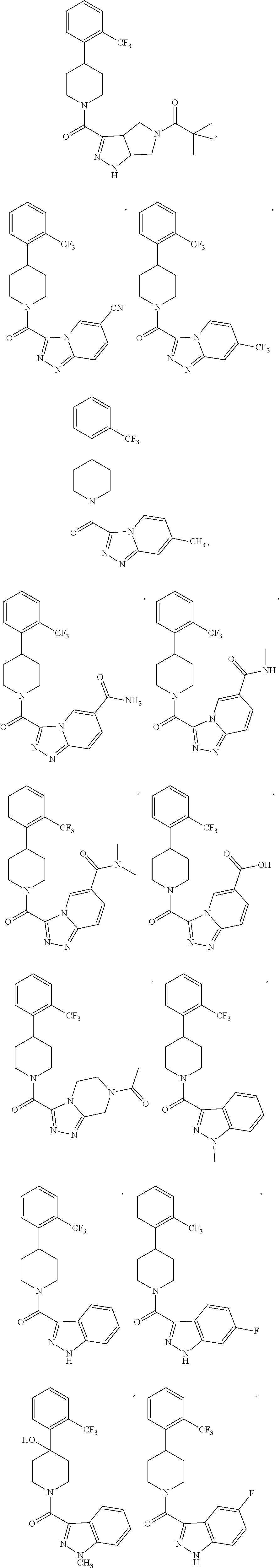

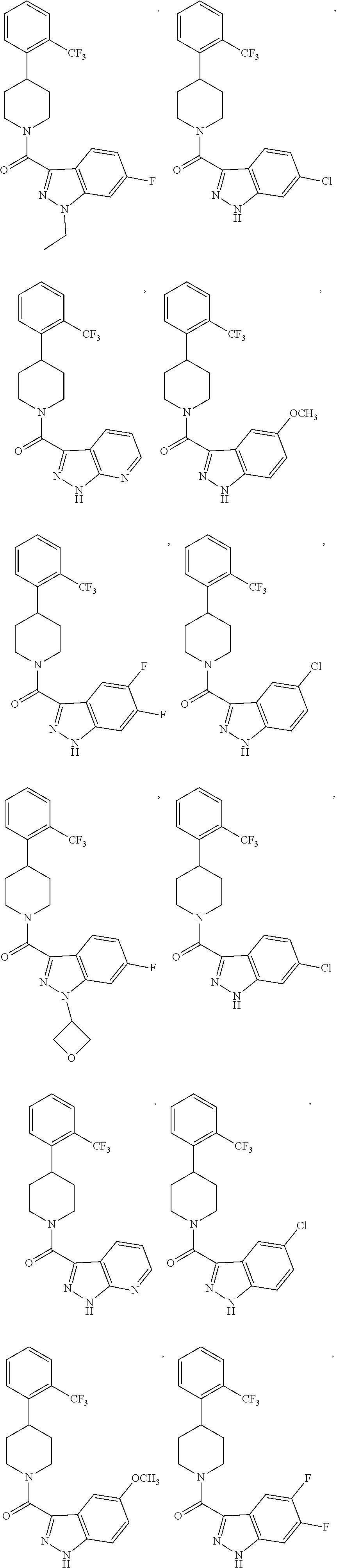

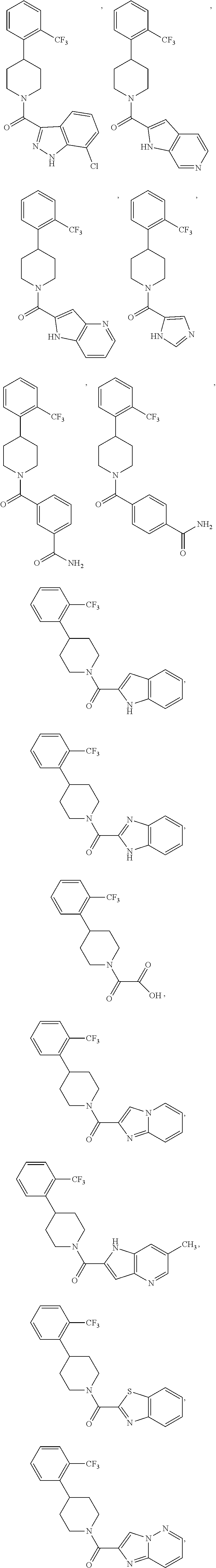

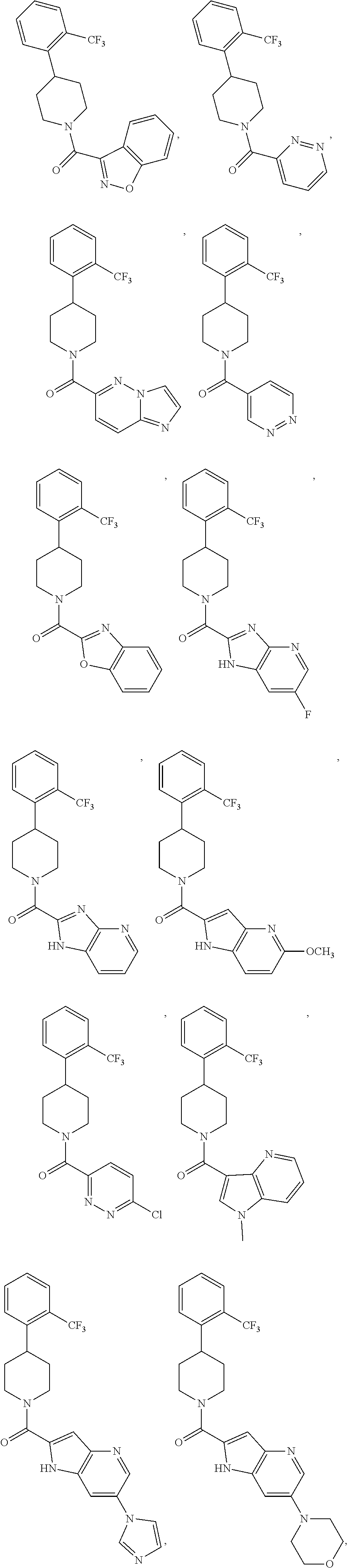

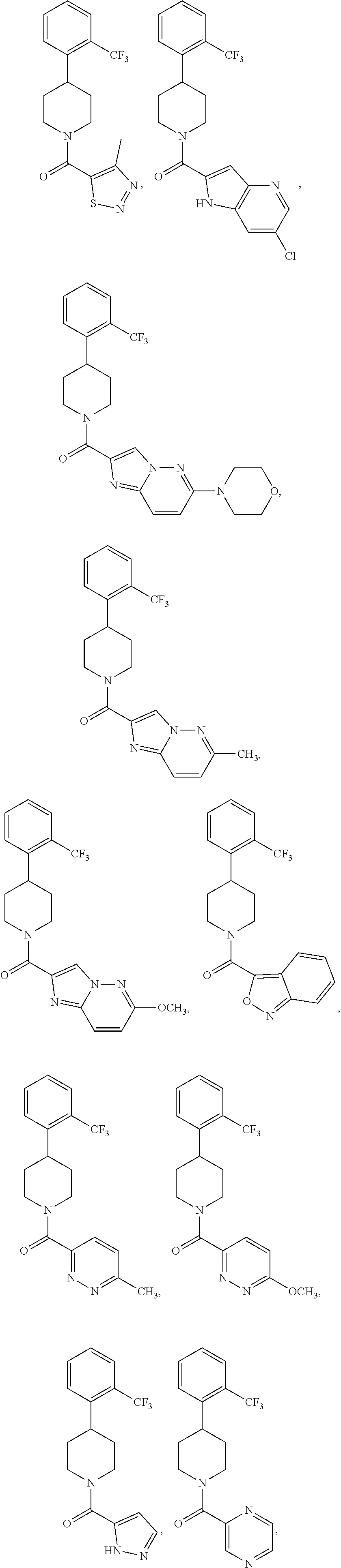

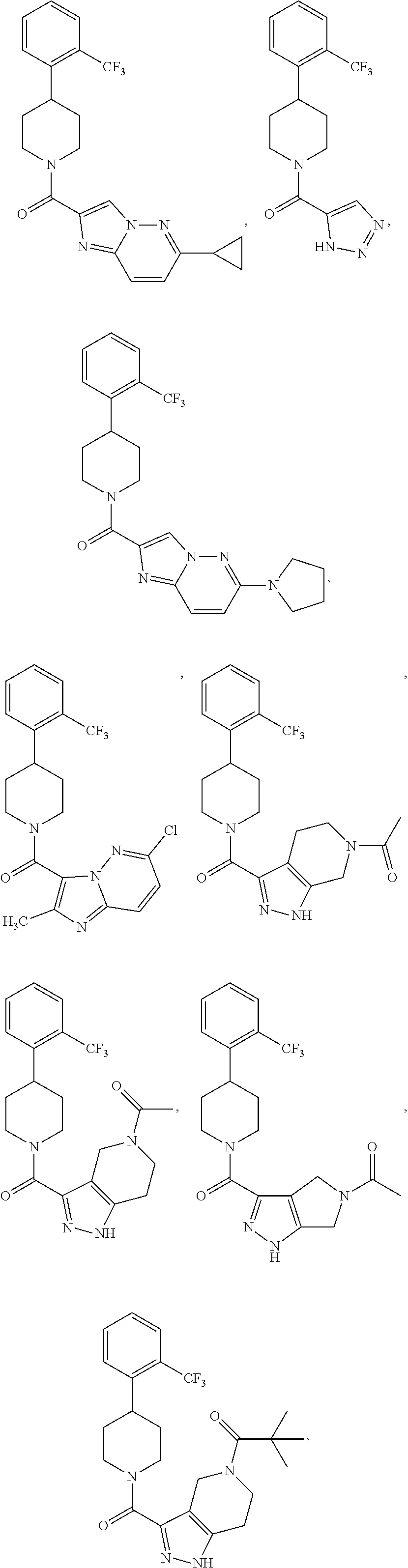

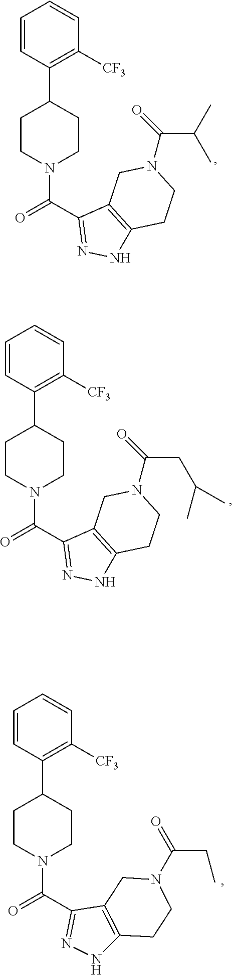 [SMILES: CC(C)C(=O)N1CCc2[nH]nc(C(=O)N3CCC(c4ccccc4C(F)(F)F)CC3)c2C1 ; CC(C)CC(=O)N1CCc2[nH]nc(C(=O)N3CCC(c4ccccc4C(F)(F)F)CC3)c2C1 ; CCC(=O)N1CCc2[nH]nc(C(=O)N3CCC(c4ccccc4C(F)(F)F)CC3)c2C1]

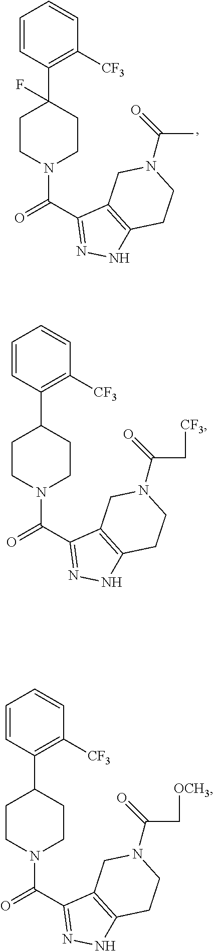 [SMILES: CC(=O)N1CCc2[nH]nc(C(=O)N3CCC(F)(c4ccccc4C(F)(F)F)CC3)c2C1 ; O=C(CC(F)(F)F)N1CCc2[nH]nc(C(=O)N3CCC(c4ccccc4C(F)(F)F)CC3)c2C1 ; COCC(=O)N1CCc2[nH]nc(C(=O)N3CCC(c4ccccc4C(F)(F)F)CC3)c2C1]

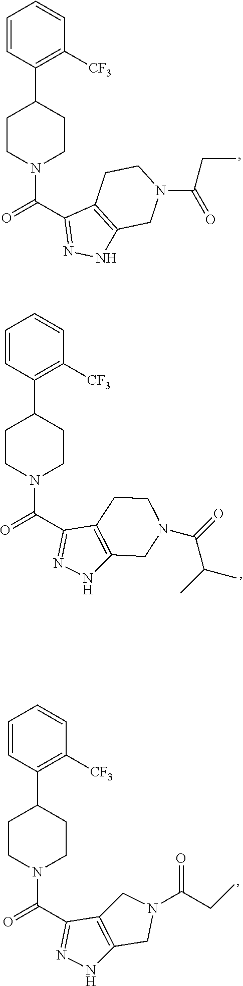 [SMILES: CCC(=O)N1CCc2c(C(=O)N3CCC(c4ccccc4C(F)(F)F)CC3)n[nH]c2C1 ; CC(C)C(=O)N1CCc2c(C(=O)N3CCC(c4ccccc4C(F)(F)F)CC3)n[nH]c2C1 ; CCC(=O)N1Cc2[nH]nc(C(=O)N3CCC(c4ccccc4C(F)(F)F)CC3)c2C1]

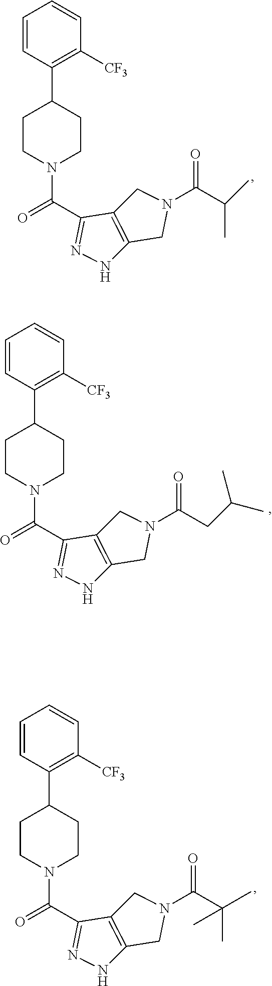 [SMILES: CC(C)C(=O)N1Cc2[nH]nc(C(=O)N3CCC(c4ccccc4C(F)(F)F)CC3)c2C1 ; CC(C)CC(=O)N1Cc2[nH]nc(C(=O)N3CCC(c4ccccc4C(F)(F)F)CC3)c2C1 ; CC(C)(C)C(=O)N1Cc2[nH]nc(C(=O)N3CCC(c4ccccc4C(F)(F)F)CC3)c2C1]

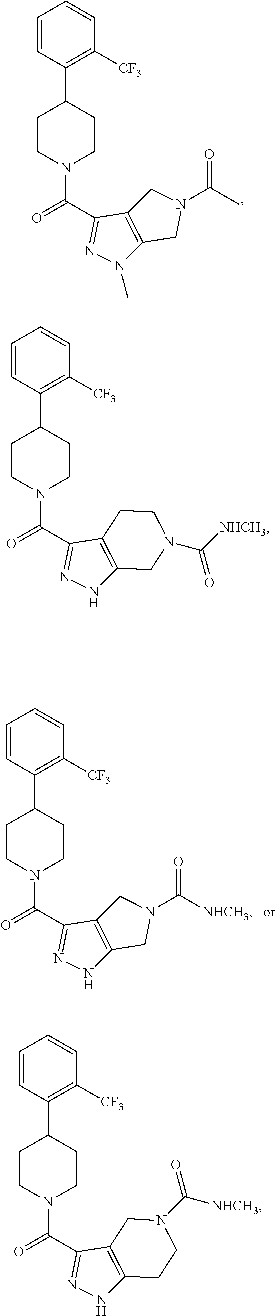 [SMILES: CC(=O)N1Cc2c(C(=O)N3CCC(c4ccccc4C(F)(F)F)CC3)nn(C)c2C1 ; CNC(=O)N1CCc2c(C(=O)N3CCC(c4ccccc4C(F)(F)F)CC3)n[nH]c2C1 ; CNC(=O)N1Cc2[nH]nc(C(=O)N3CCC(c4ccccc4C(F)(F)F)CC3)c2C1 ; CNC(=O)N1CCc2[nH]nc(C(=O)N3CCC(c4ccccc4C(F)(F)F)CC3)c2C1]

or a pharmaceutically acceptable salt thereof.

In some embodiments, the selective RBP4 antagonist is

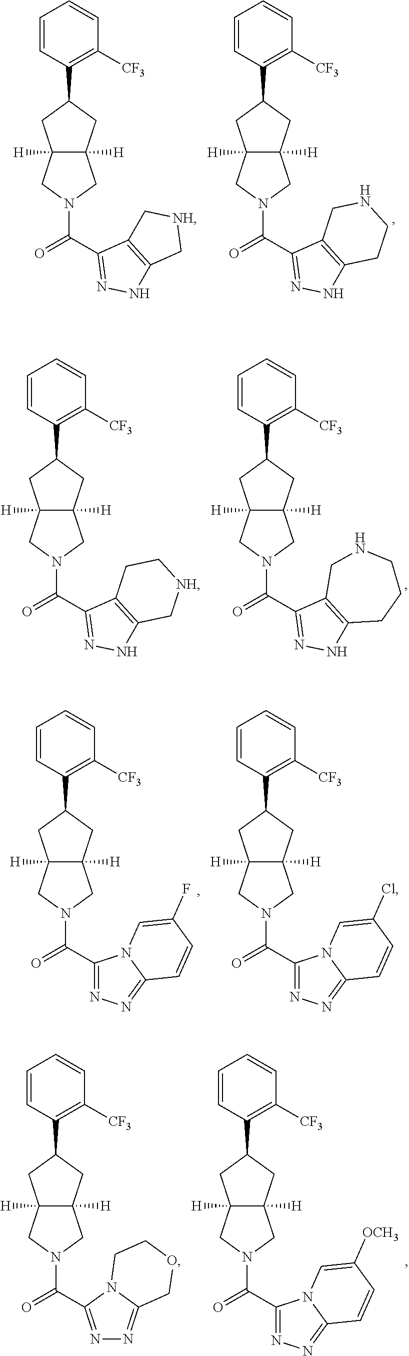

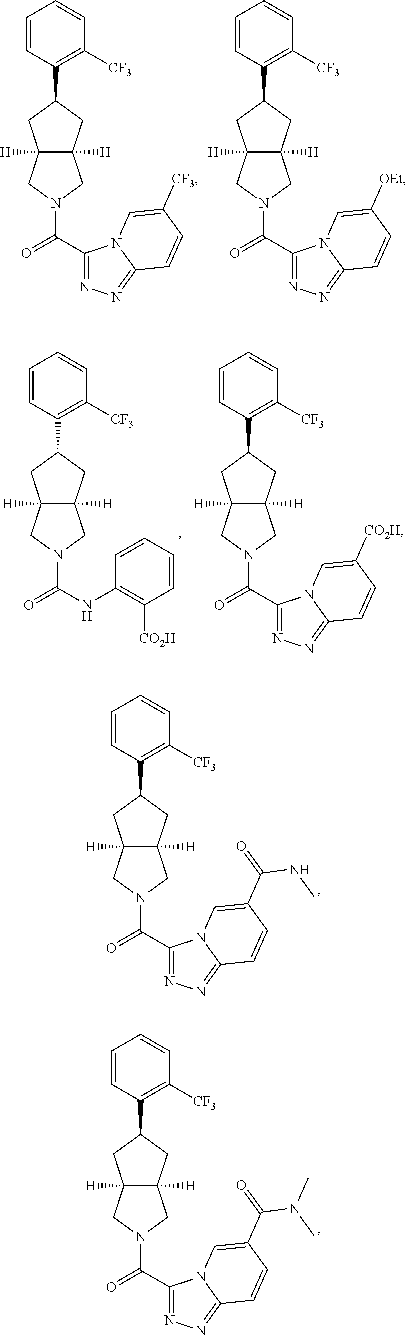

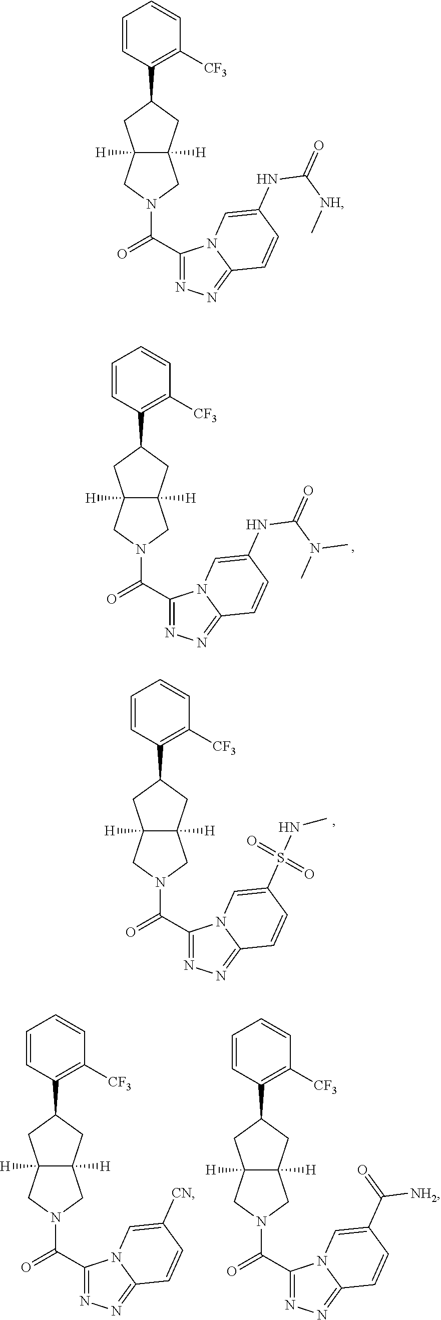

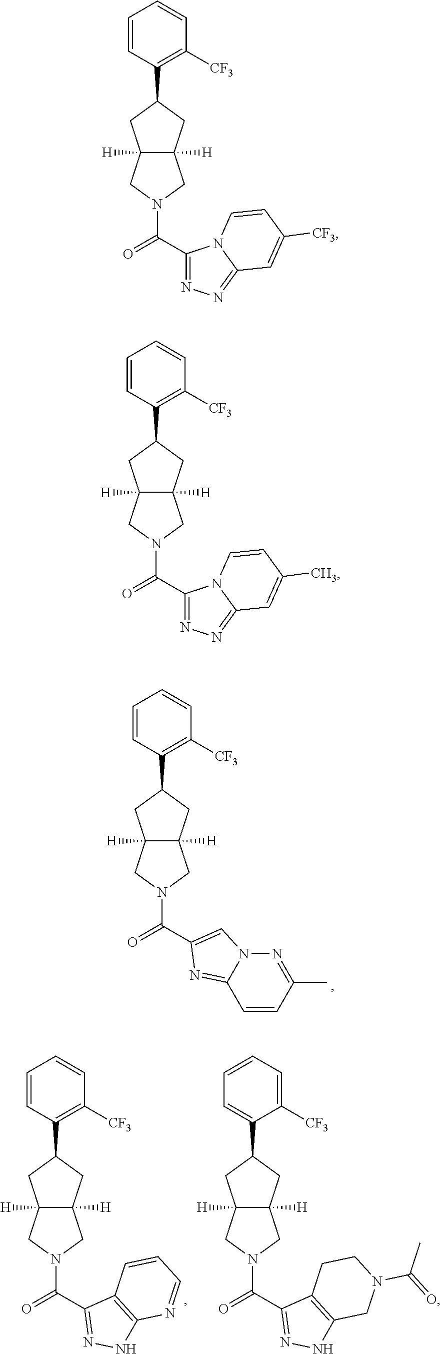

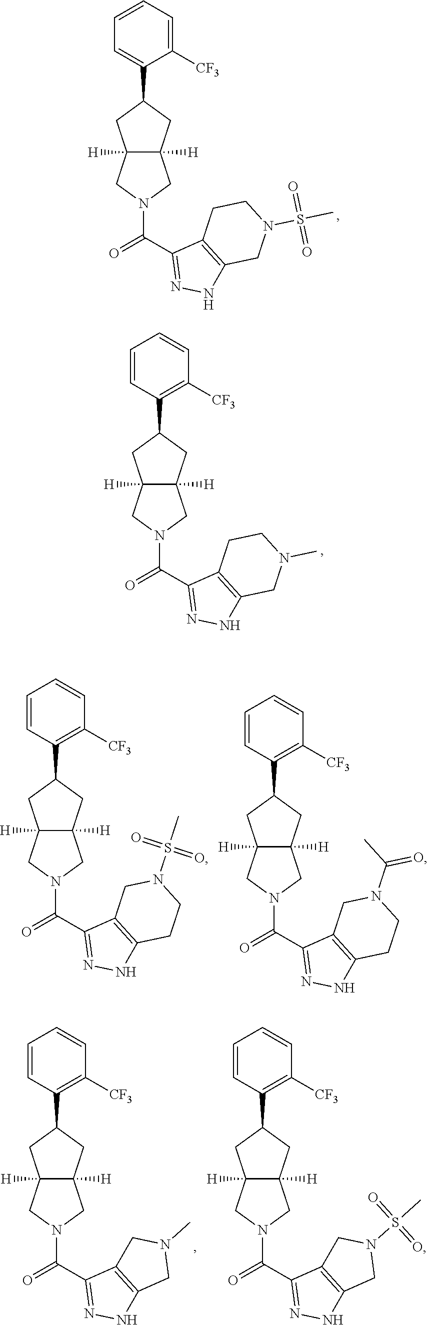

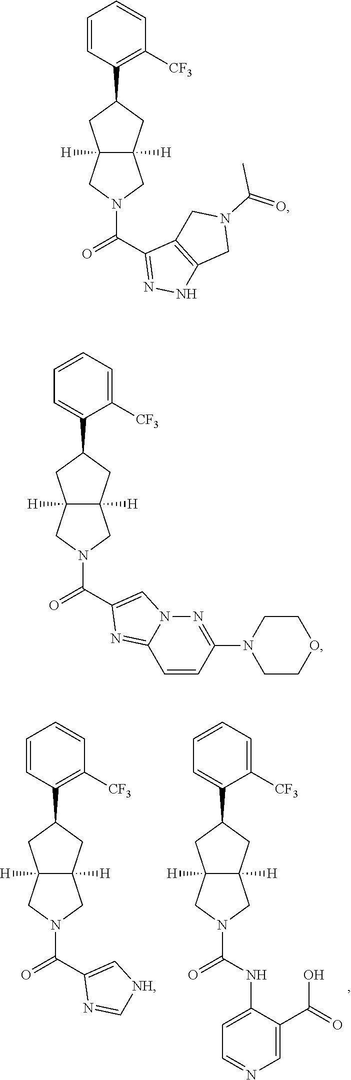 [SMILES: CC(=O)N1Cc2[nH]nc(C(=O)N3C[C@H]4C[C@@H](c5ccccc5C(F)(F)F)C[C@H]4C3)c2C1 ; O=C(c1cn2nc(N3CCOCC3)ccc2n1)N1C[C@H]2C[C@@H](c3ccccc3C(F)(F)F)C[C@H]2C1 ; O=C(c1c[nH]cn1)N1C[C@H]2C[C@@H](c3ccccc3C(F)(F)F)C[C@H]2C1 ; O=C(O)c1cnccc1NC(=O)N1C[C@H]2C[C@@H](c3ccccc3C(F)(F)F)C[C@H]2C1]

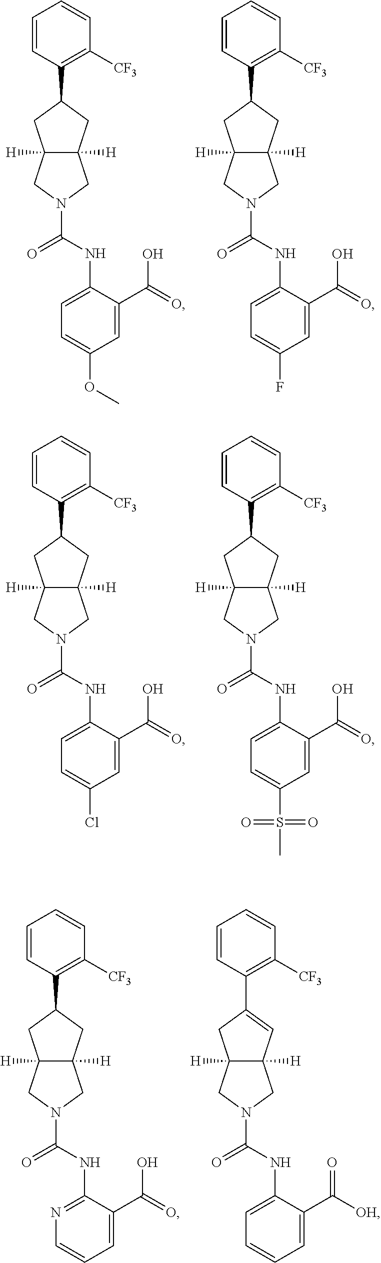 [SMILES: COc1ccc(NC(=O)N2C[C@H]3C[C@@H](c4ccccc4C(F)(F)F)C[C@H]3C2)c(C(=O)O)c1 ; O=C(O)c1cc(F)ccc1NC(=O)N1C[C@H]2C[C@@H](c3ccccc3C(F)(F)F)C[C@H]2C1 ; O=C(O)c1cc(Cl)ccc1NC(=O)N1C[C@H]2C[C@@H](c3ccccc3C(F)(F)F)C[C@H]2C1 ; CS(=O)(=O)c1ccc(NC(=O)N2C[C@H]3C[C@@H](c4ccccc4C(F)(F)F)C[C@H]3C2)c(C(=O)O)c1 ; O=C(O)c1cccnc1NC(=O)N1C[C@H]2C[C@@H](c3ccccc3C(F)(F)F)C[C@H]2C1 ; O=C(O)c1ccccc1NC(=O)N1C[C@H]2CC(c3ccccc3C(F)(F)F)=C[C@H]2C1]

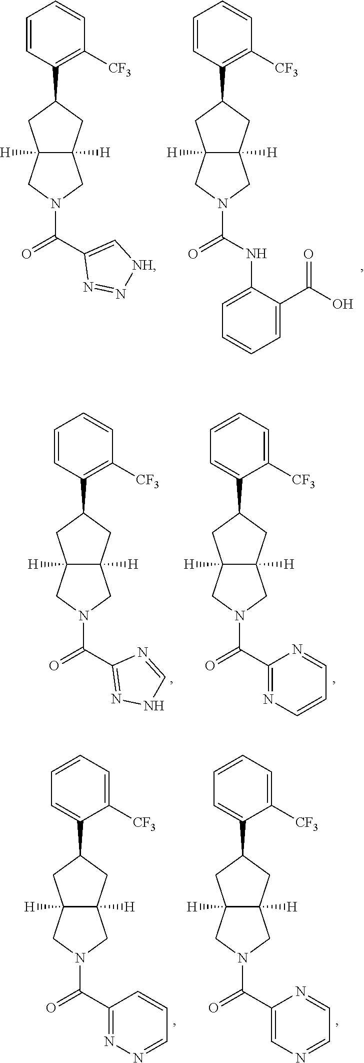 [SMILES: O=C(c1c[nH]nn1)N1C[C@H]2C[C@@H](c3ccccc3C(F)(F)F)C[C@H]2C1 ; O=C(O)c1ccccc1NC(=O)N1C[C@H]2C[C@@H](c3ccccc3C(F)(F)F)C[C@H]2C1 ; O=C(c1nc[nH]n1)N1C[C@H]2C[C@@H](c3ccccc3C(F)(F)F)C[C@H]2C1 ; O=C(c1ncccn1)N1C[C@H]2C[C@@H](c3ccccc3C(F)(F)F)C[C@H]2C1 ; O=C(c1cccnn1)N1C[C@H]2C[C@@H](c3ccccc3C(F)(F)F)C[C@H]2C1 ; O=C(c1cnccn1)N1C[C@H]2C[C@@H](c3ccccc3C(F)(F)F)C[C@H]2C1]

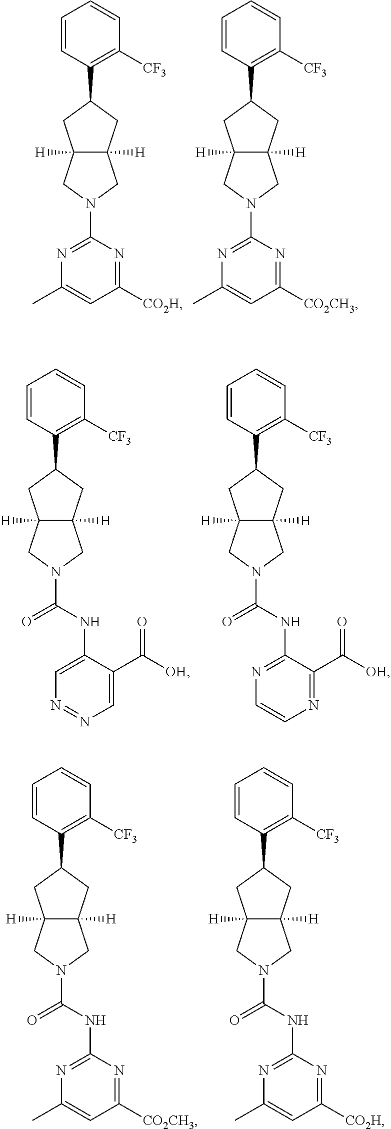

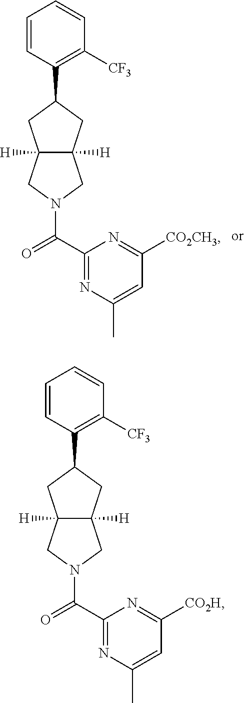 [SMILES: COC(=O)c1cc(C)nc(C(=O)N2C[C@H]3C[C@@H](c4ccccc4C(F)(F)F)C[C@H]3C2)n1 ; "or" ; Cc1cc(C(=O)O)nc(C(=O)N2C[C@H]3C[C@@H](c4ccccc4C(F)(F)F)C[C@H]3C2)n1]

or a pharmaceutically acceptable salt thereof.

In some embodiments, the selective RBP4 antagonist is

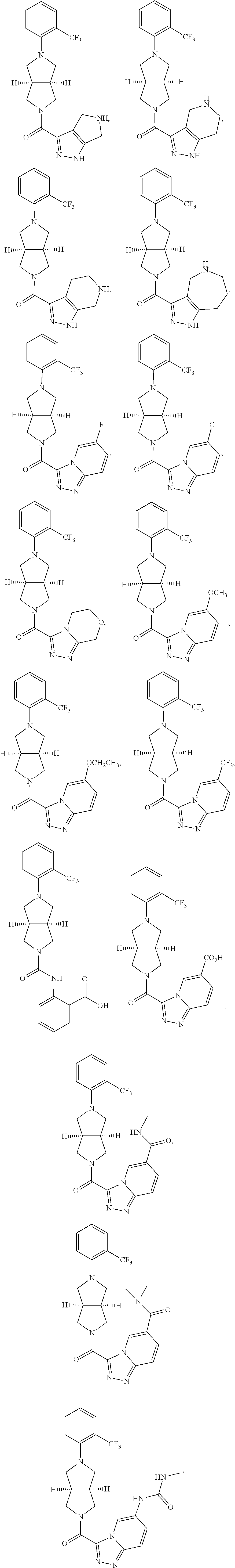

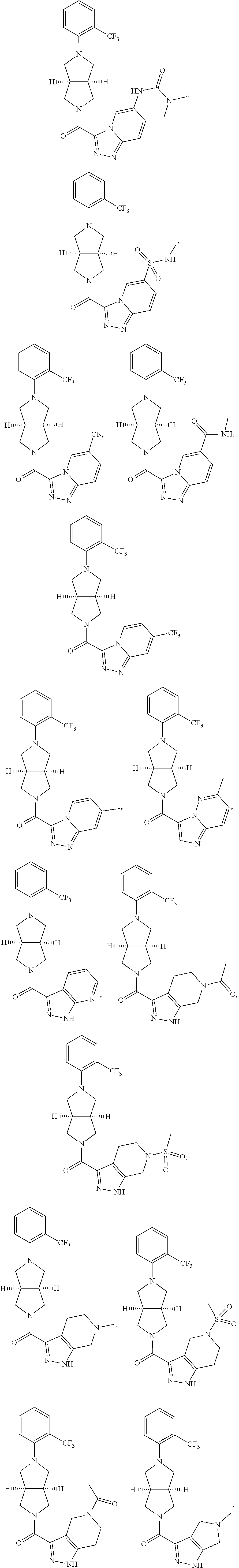

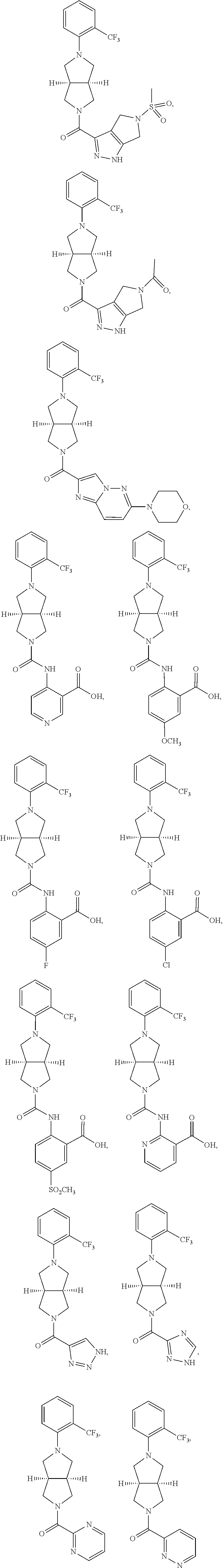

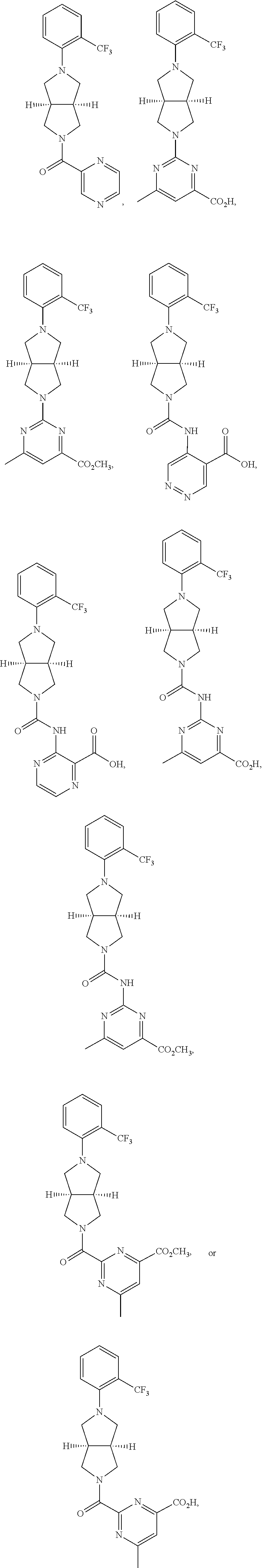

or a pharmaceutically acceptable salt thereof.

In some embodiments, the selective RBP4 antagonist is

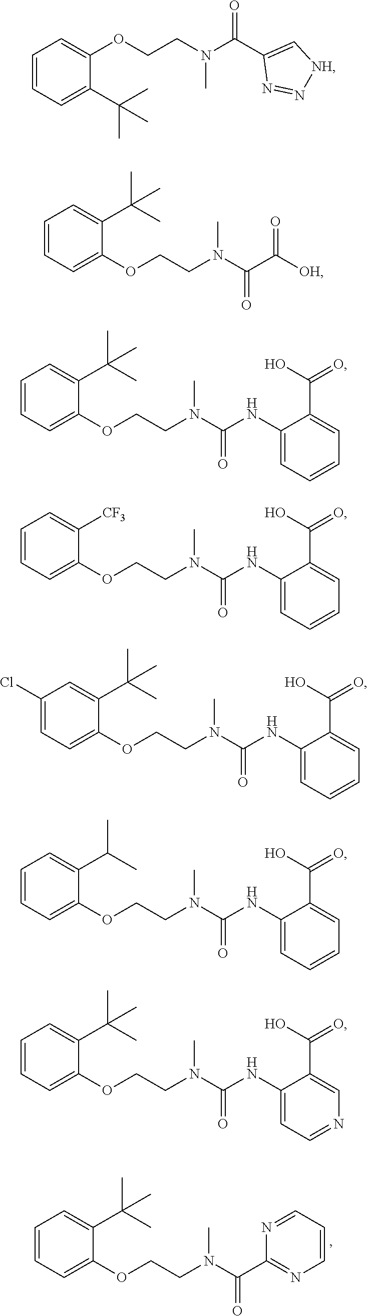

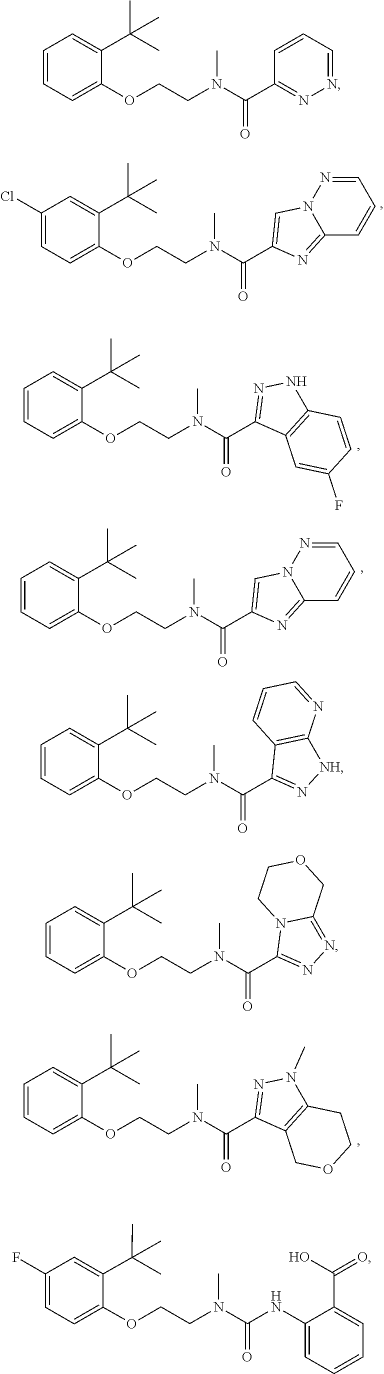

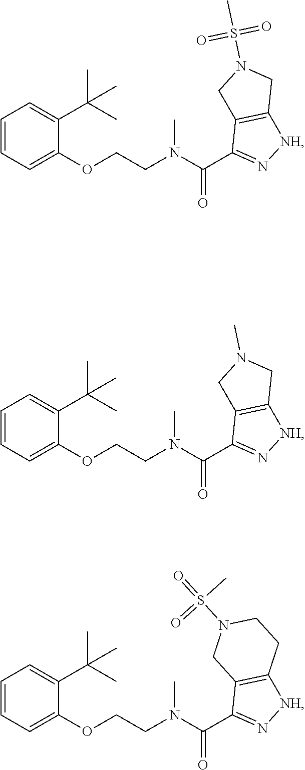 [SMILES: CN(CCOc1ccccc1C(C)(C)C)C(=O)c1n[nH]c2c1CN(S(C)(=O)=O)C2 ; CN1Cc2[nH]nc(C(=O)N(C)CCOc3ccccc3C(C)(C)C)c2C1 ; CN(CCOc1ccccc1C(C)(C)C)C(=O)c1n[nH]c2c1CN(S(C)(=O)=O)CC2]

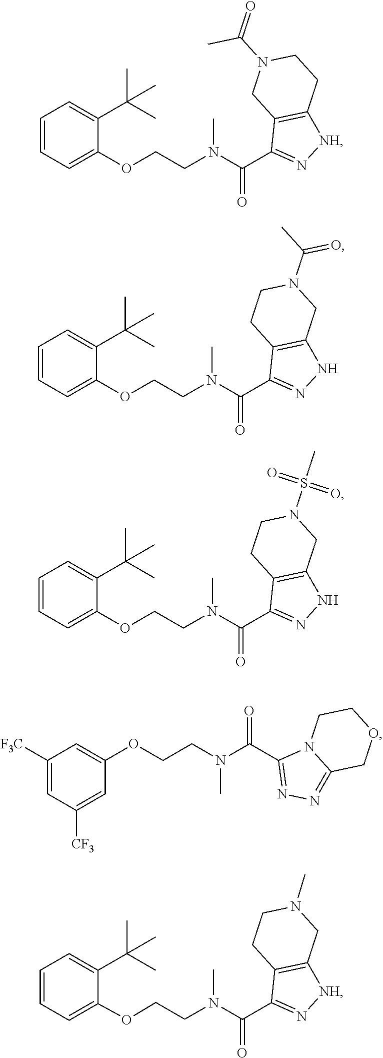

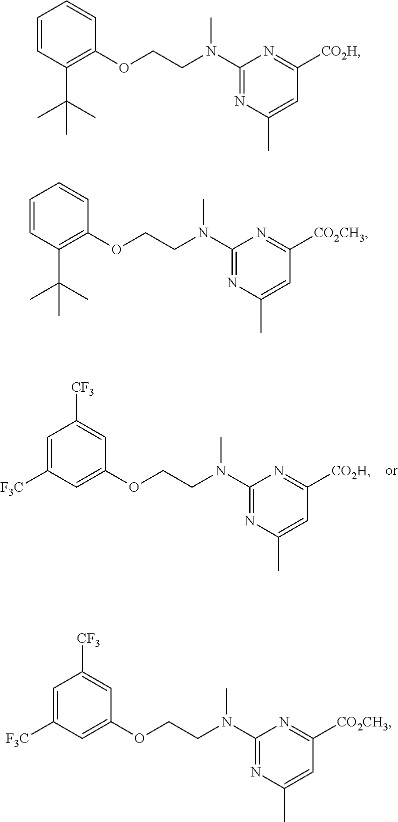 [SMILES: Cc1cc(C(=O)O)nc(N(C)CCOc2ccccc2C(C)(C)C)n1 ; COC(=O)c1cc(C)nc(N(C)CCOc2ccccc2C(C)(C)C)n1 ; Cc1cc(C(=O)O)nc(N(C)CCOc2cc(C(F)(F)F)cc(C(F)(F)F)c2)n1 ; COC(=O)c1cc(C)nc(N(C)CCOc2cc(C(F)(F)F)cc(C(F)(F)F)c2)n1]

or a pharmaceutically acceptable salt thereof.

In some embodiments, the selective RBP4 antagonist is

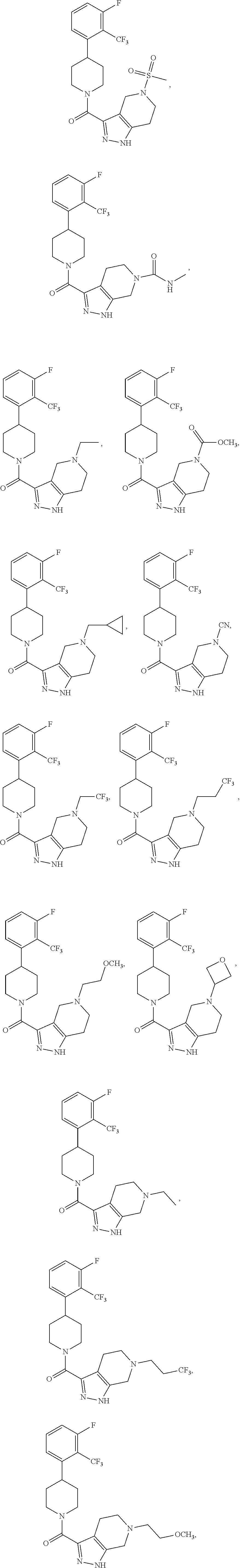

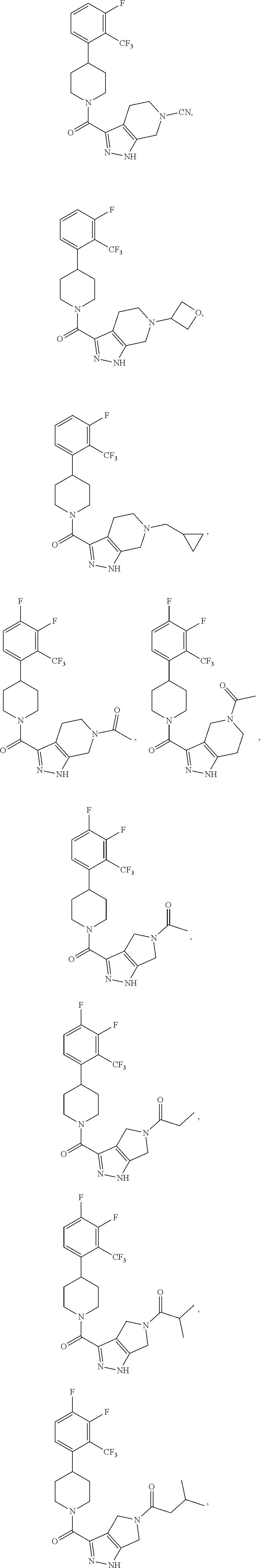

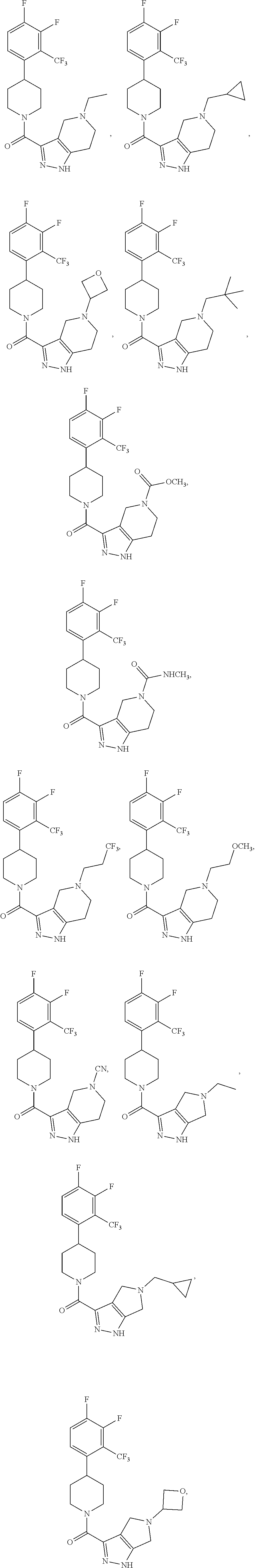

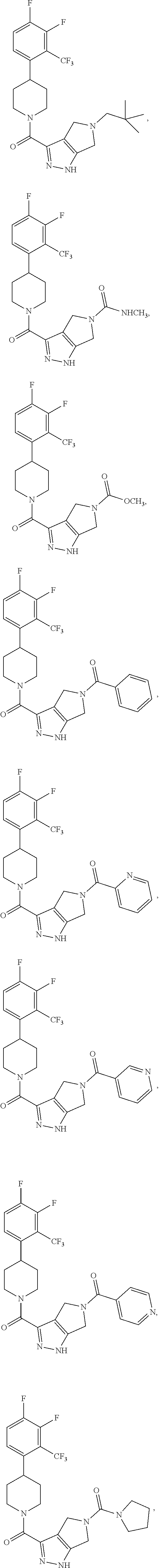

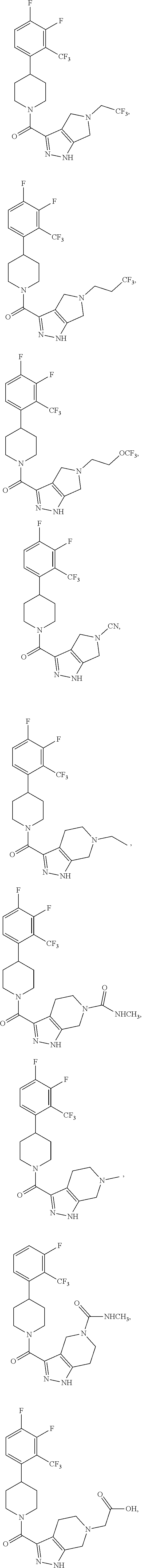

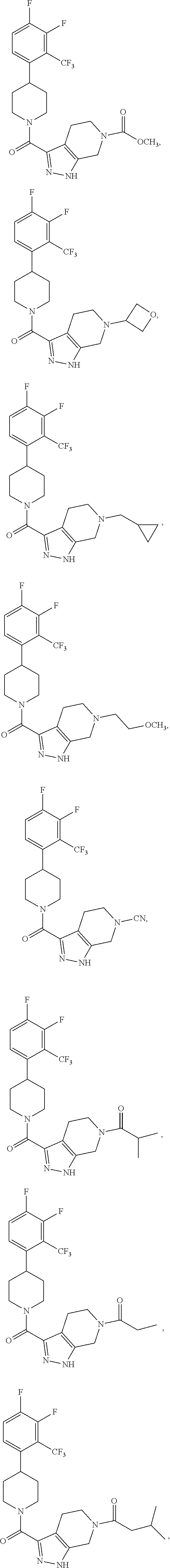

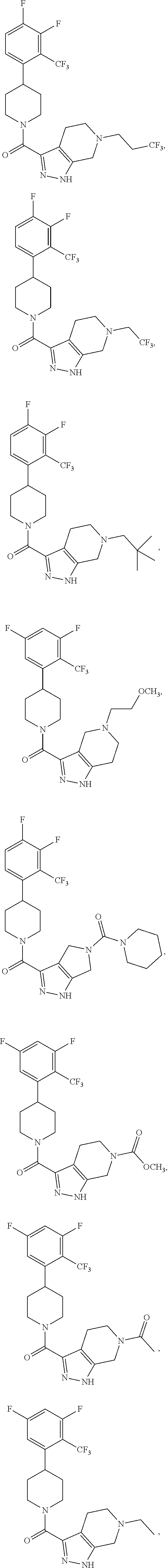

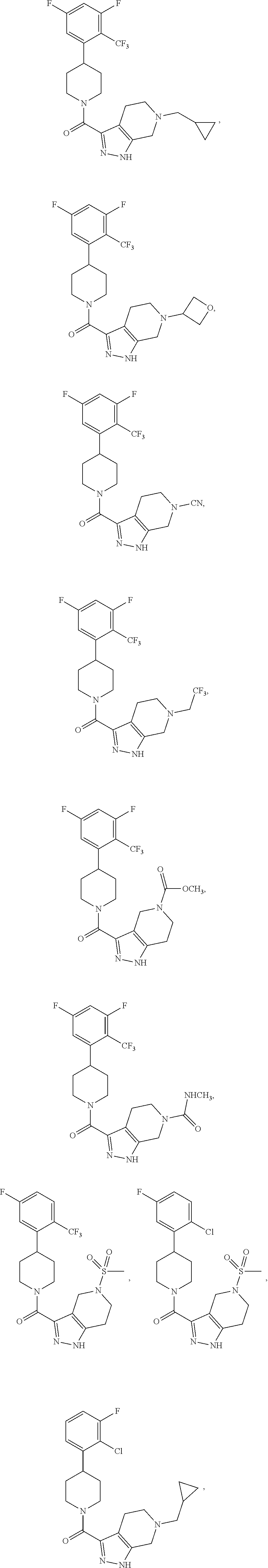

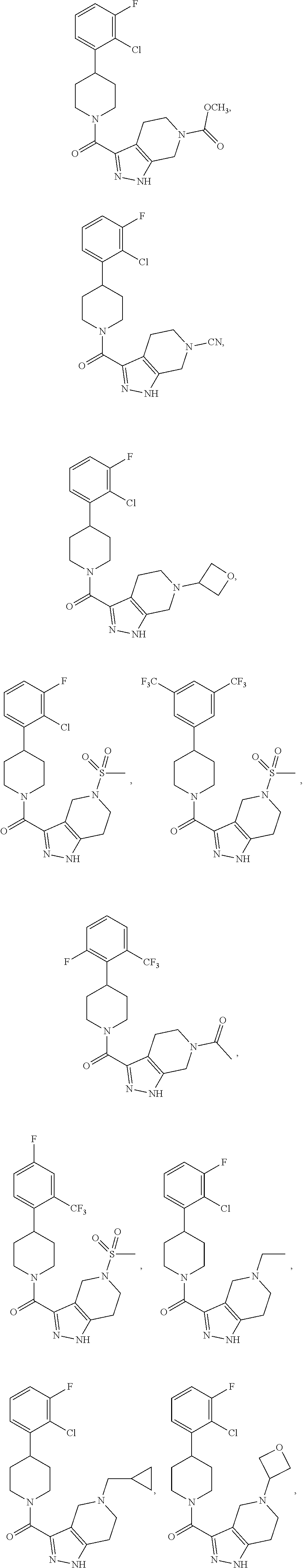

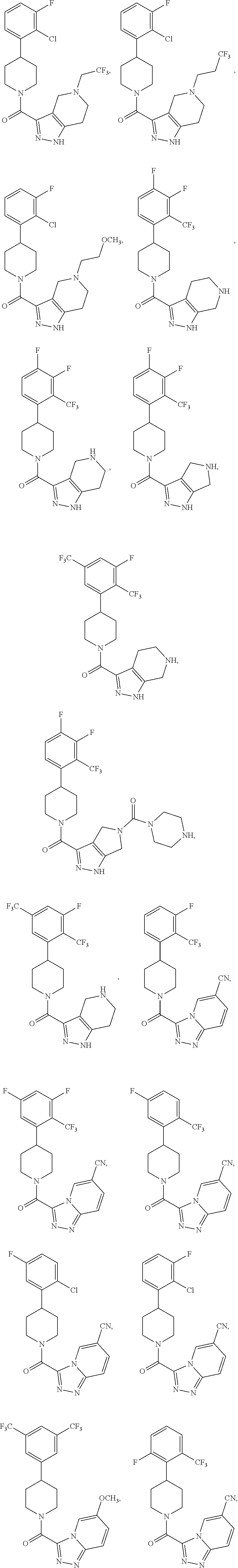

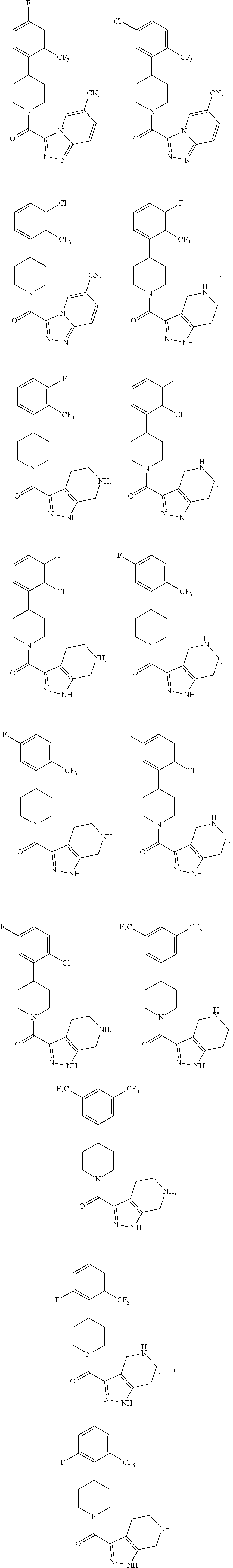

or a pharmaceutically acceptable salt thereof.

In some embodiments, the selective RBP4 antagonist is

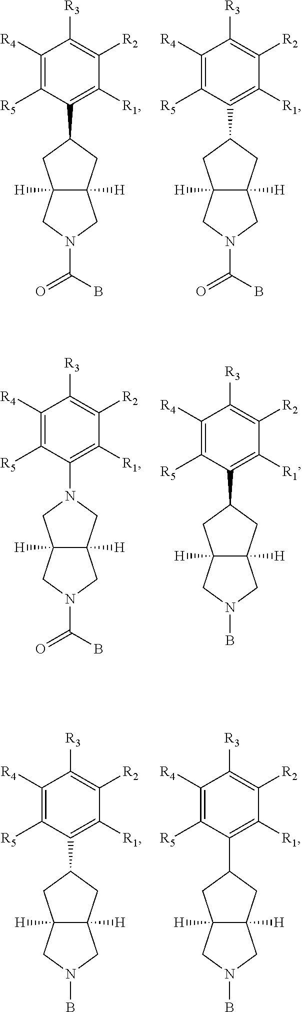

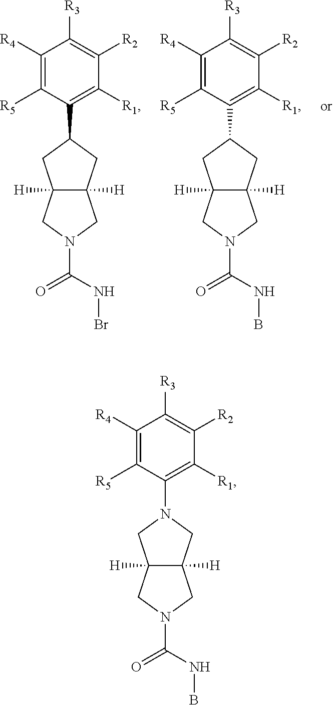

or a pharmaceutically acceptable salt thereof.

In some embodiments, the selective RBP4 antagonist is

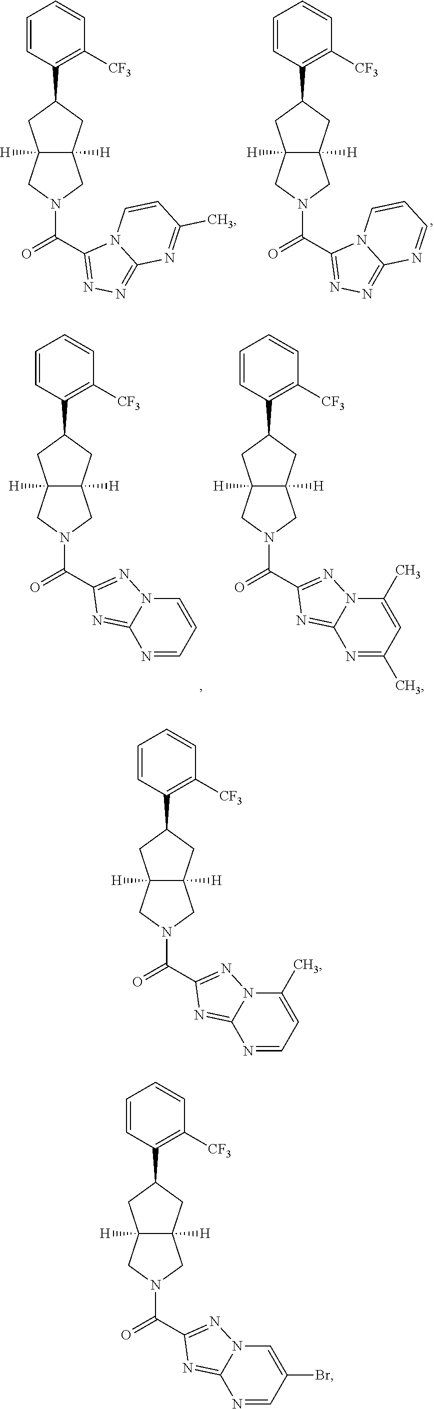 [SMILES: Cc1ccn2c(C(=O)N3C[C@H]4C[C@@H](c5ccccc5C(F)(F)F)C[C@H]4C3)nnc2n1 ; O=C(c1nnc2ncccn12)N1C[C@H]2C[C@@H](c3ccccc3C(F)(F)F)C[C@H]2C1 ; O=C(c1nc2ncccn2n1)N1C[C@H]2C[C@@H](c3ccccc3C(F)(F)F)C[C@H]2C1 ; Cc1cc(C)n2nc(C(=O)N3C[C@H]4C[C@@H](c5ccccc5C(F)(F)F)C[C@H]4C3)nc2n1 ; Cc1ccnc2nc(C(=O)N3C[C@H]4C[C@@H](c5ccccc5C(F)(F)F)C[C@H]4C3)nn12 ; O=C(c1nc2ncc(Br)cn2n1)N1C[C@H]2C[C@@H](c3ccccc3C(F)(F)F)C[C@H]2C1]

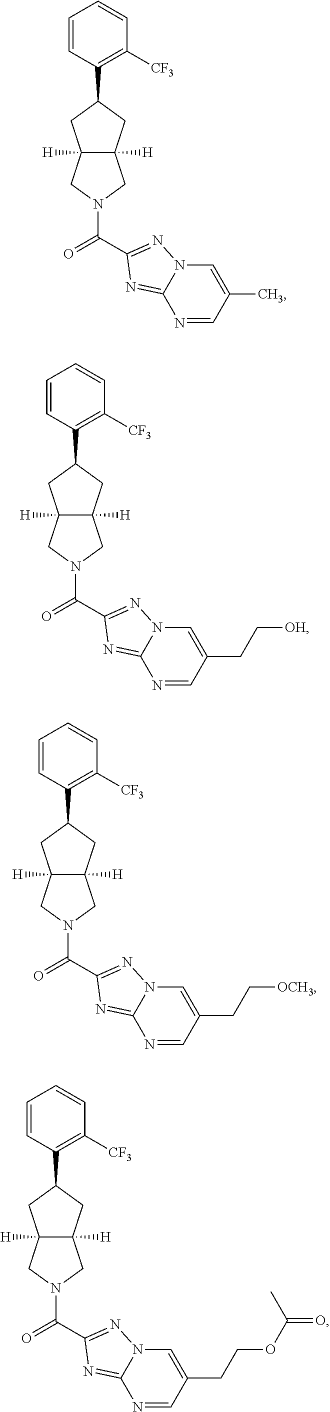 [SMILES: Cc1cnc2nc(C(=O)N3C[C@H]4C[C@@H](c5ccccc5C(F)(F)F)C[C@H]4C3)nn2c1 ; O=C(c1nc2ncc(CCO)cn2n1)N1C[C@H]2C[C@@H](c3ccccc3C(F)(F)F)C[C@H]2C1 ; COCCc1cnc2nc(C(=O)N3C[C@H]4C[C@@H](c5ccccc5C(F)(F)F)C[C@H]4C3)nn2c1 ; CC(=O)OCCc1cnc2nc(C(=O)N3C[C@H]4C[C@@H](c5ccccc5C(F)(F)F)C[C@H]4C3)nn2c1]

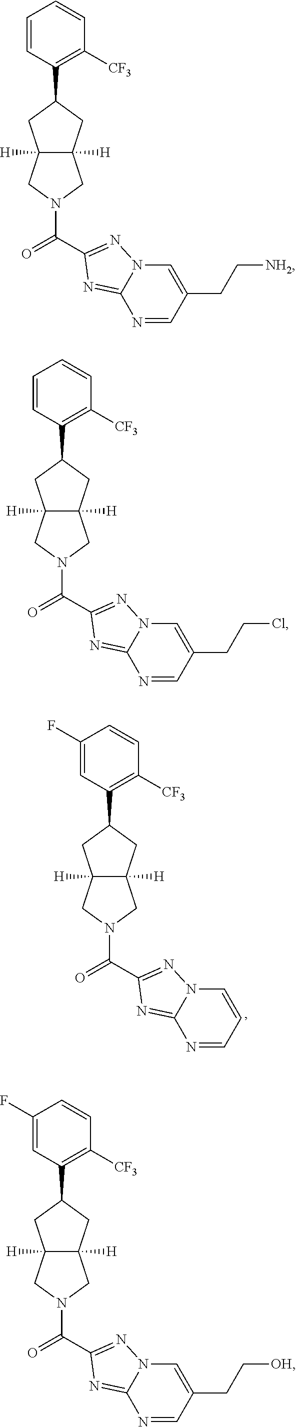 [SMILES: NCCc1cnc2nc(C(=O)N3C[C@H]4C[C@@H](c5ccccc5C(F)(F)F)C[C@H]4C3)nn2c1 ; O=C(c1nc2ncc(CCCl)cn2n1)N1C[C@H]2C[C@@H](c3ccccc3C(F)(F)F)C[C@H]2C1 ; O=C(c1nc2ncccn2n1)N1C[C@H]2C[C@@H](c3cc(F)ccc3C(F)(F)F)C[C@H]2C1 ; O=C(c1nc2ncc(CCO)cn2n1)N1C[C@H]2C[C@@H](c3cc(F)ccc3C(F)(F)F)C[C@H]2C1]

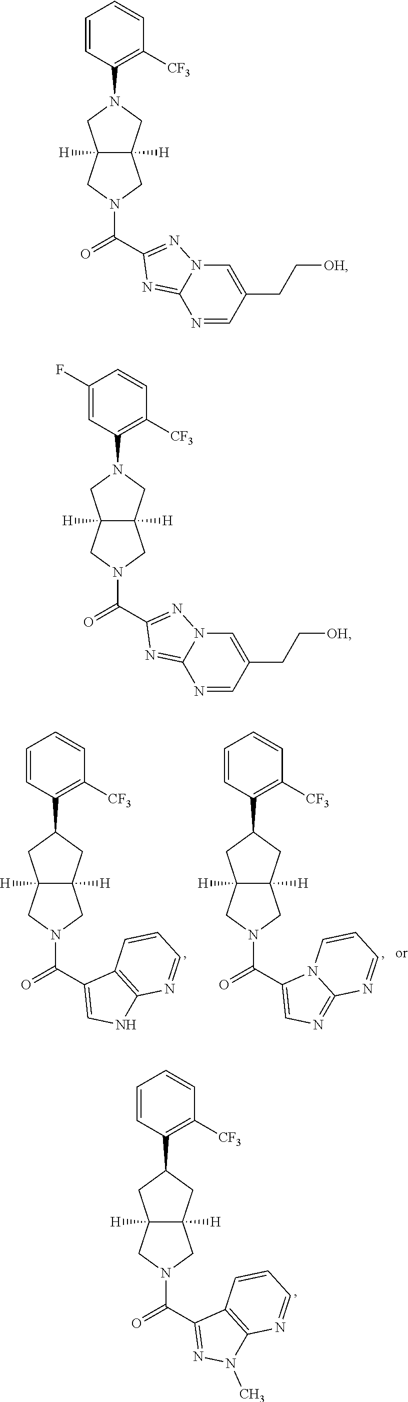

or a pharmaceutically acceptable salt thereof.

In some embodiments, the selective RBP4 antagonist is the compound having the structure

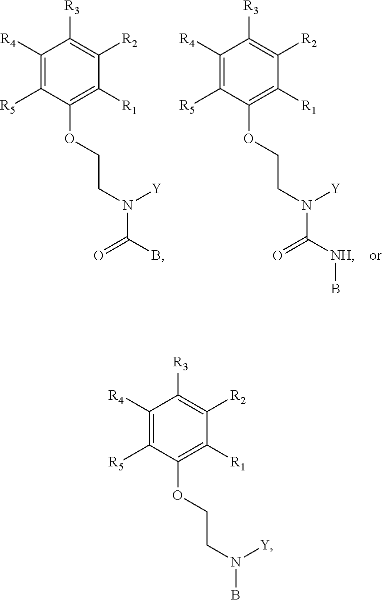

or a pharmaceutically acceptable salt thereof.

In some embodiments, the selective RBP4 antagonist is

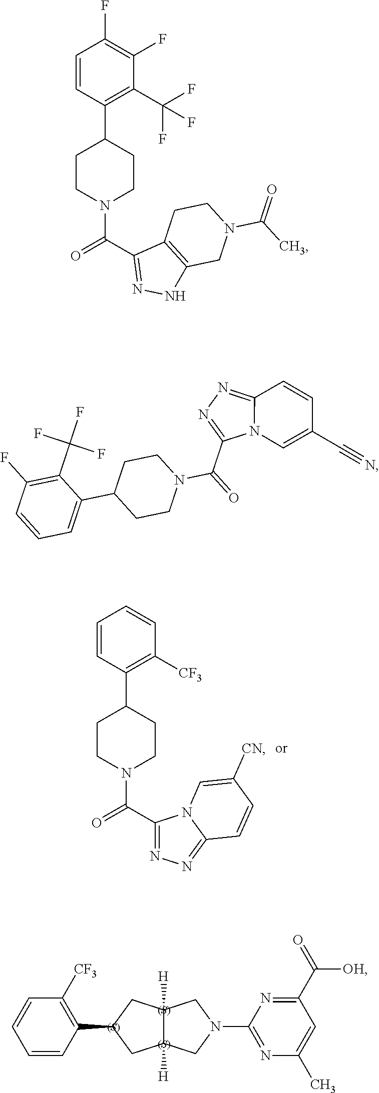

or a pharmaceutically acceptable salt thereof.

In some embodiments, P is

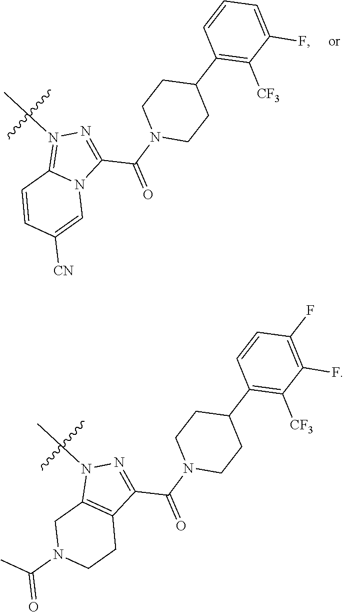

In some embodiments, Q is

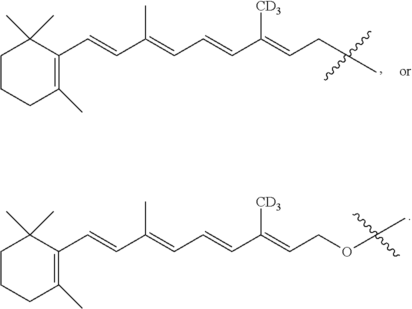 [SMILES: *C/C=C(/C=C/C=C(C)/C=C/C1=C(C)CCCC1(C)C)C([2H])([2H])[2H] ; *OC/C=C(/C=C/C=C(C)/C=C/C1=C(C)CCCC1(C)C)C([2H])([2H])[2H]]

In some embodiments, M is

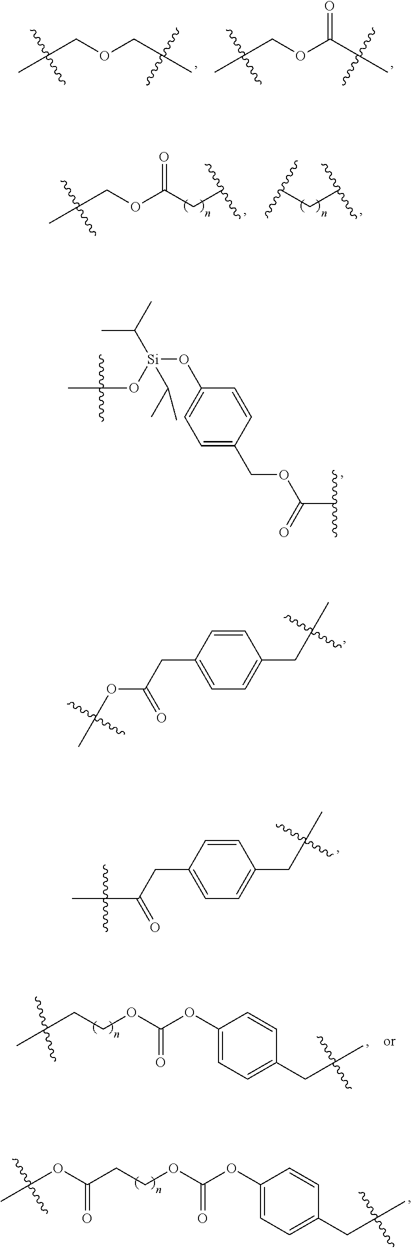

wherein n is 0-20.

In some embodiments, the present invention provides a compound having the following structure:

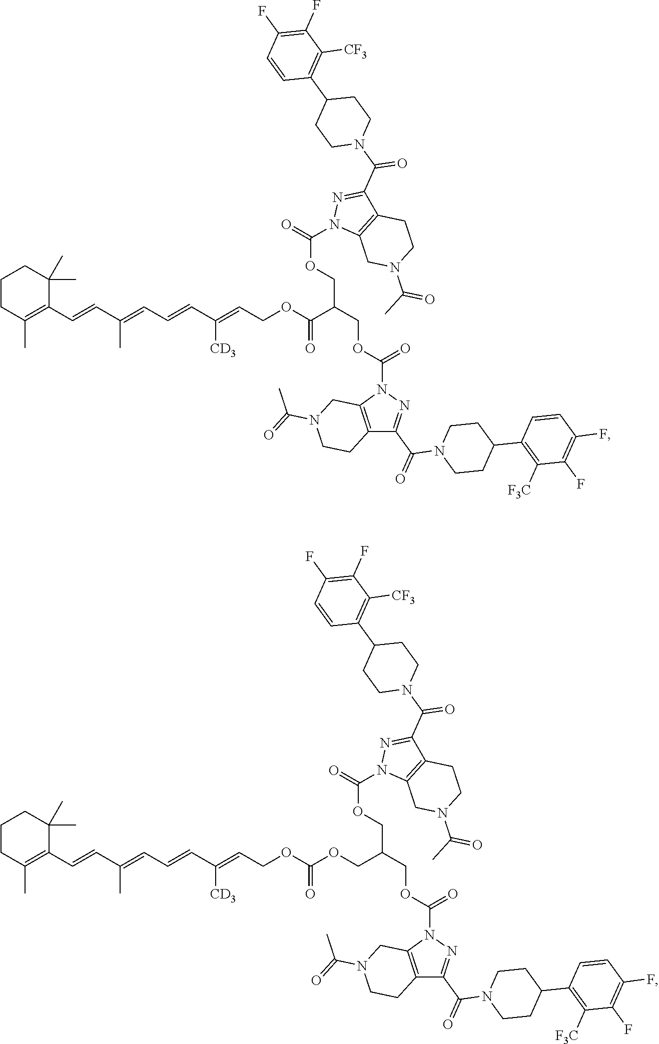

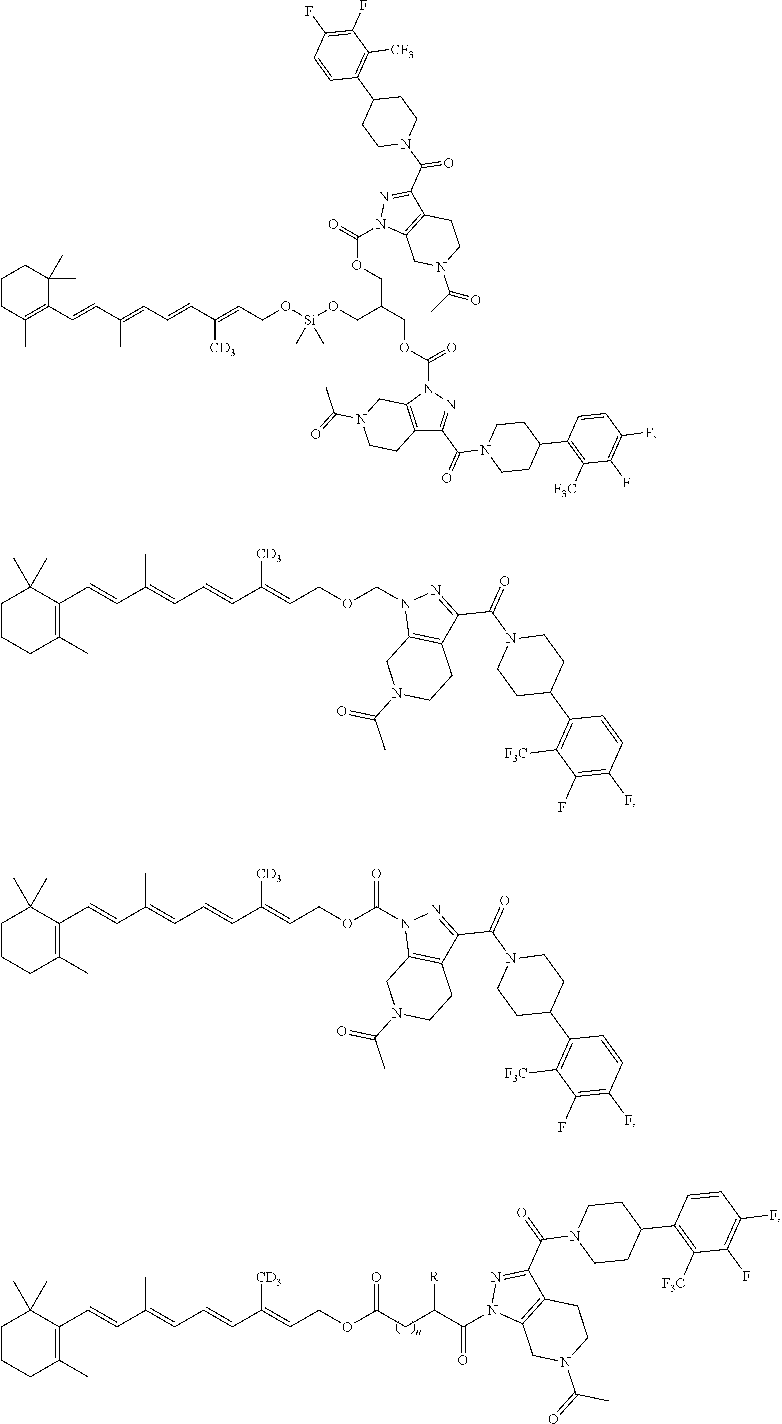

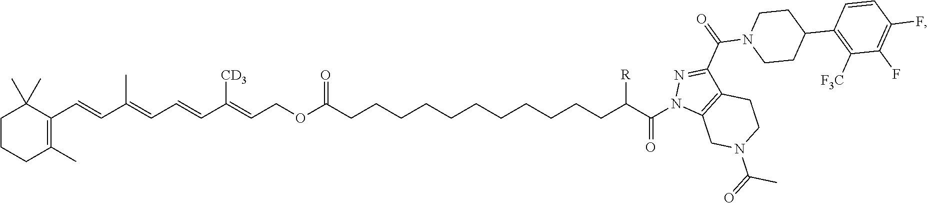

wherein n is 0-20; R is H. Trp, Phe, Leu, or Tyr;

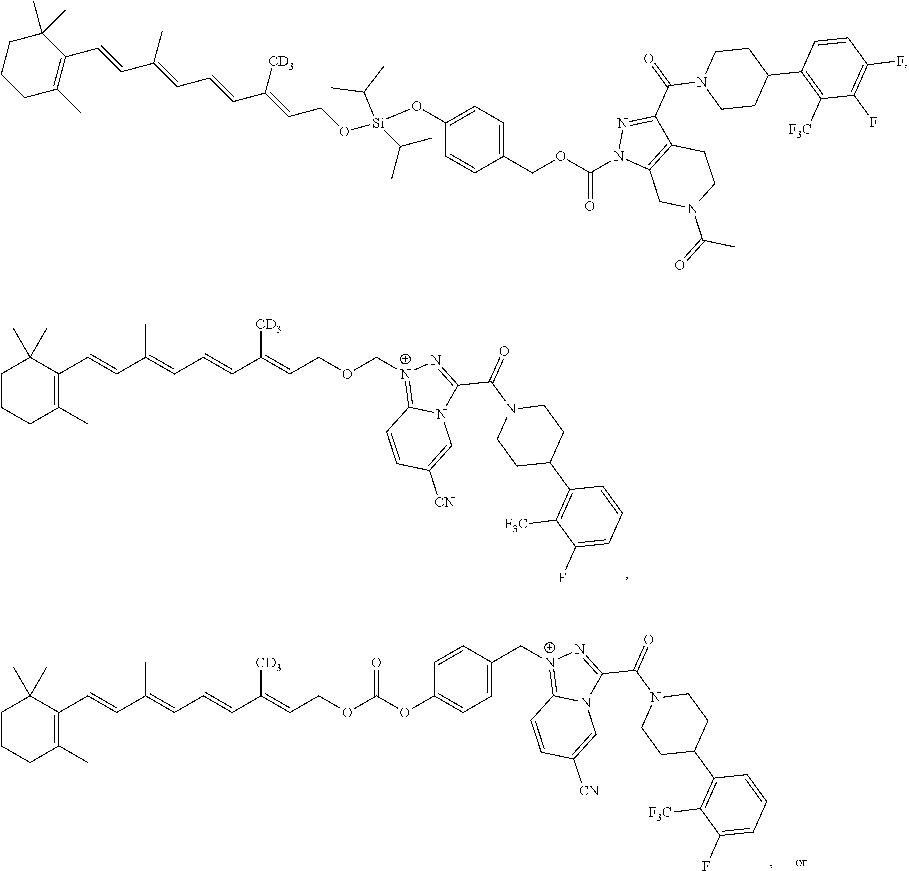

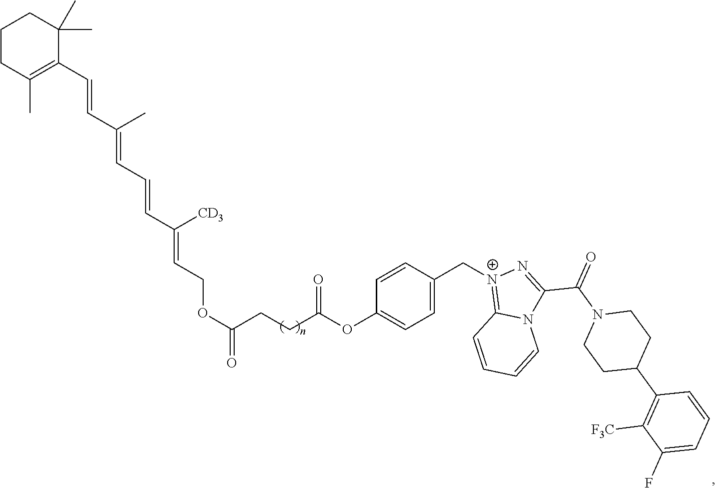

wherein n is 0-20.

In some embodiments, the selective RBP4 antagonist is the compound having the structure:

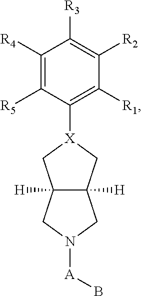

wherein
R₁, R₂, R₃, R₄, and R₅ are each independently H, halogen, CF₃, or C₁-C₄ alkyl;
X is N or CR₆,
   wherein R₆ is H, OH, or halogen;
A is absent or present, and when present, is or

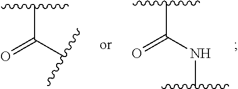

B has the structure:

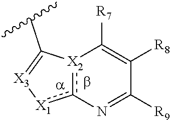

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

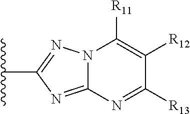

wherein
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound having the structure

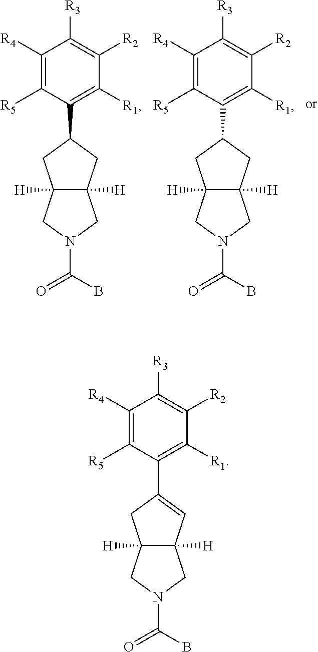

In some embodiments, the selective RBP4 antagonist is the compound having the structure

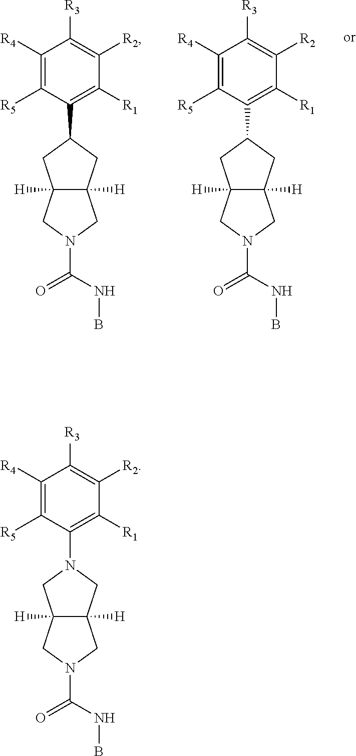

In some embodiments, the selective RBP4 antagonist is the compound having the structure

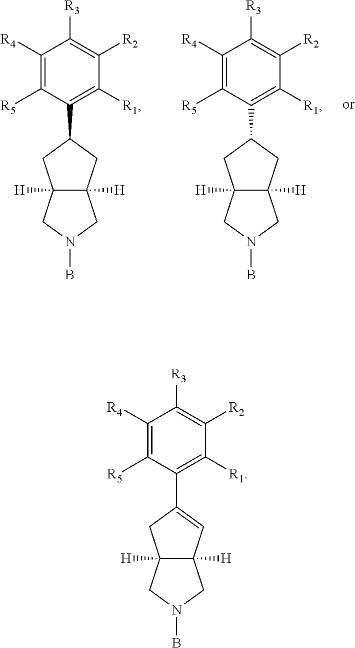

In some embodiments, the selective RBP4 antagonist is the compound having the structure:

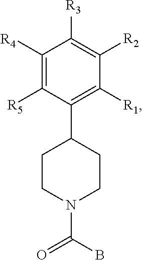

wherein
R₁, R₂, R₃, R₄, and R₅ are each independently H, halogen, CF₃, or C₁-C₄ alkyl; and
B has the structure:

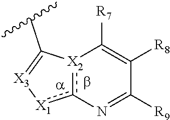

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

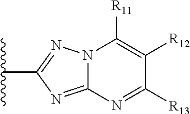

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
   or a pharmaceutically acceptable salt thereof.

In some embodiments, the selective RBP4 antagonist is the compound having the structure:

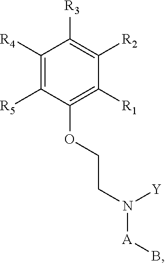

wherein
R₁, R₂, R₃, R₄, and R₅ are each independently H, halogen, CF₃, or C₁-C₄ alkyl;
Y is alkyl;

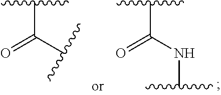

A is absent or present, and when present, is or 0 v; and
B has the structure:

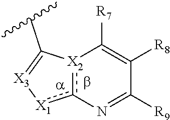

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

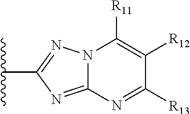

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
   or a pharmaceutically acceptable salt thereof.

In some embodiments, the selective RBP4 antagonist is the compound having the structure

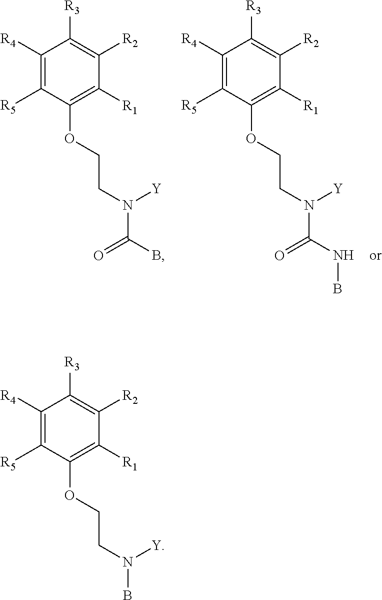

In some embodiments, the selective RBP4 antagonist is the compound wherein B has the structure:

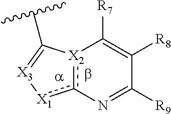

wherein
α and β are each a bond that is present or absent;
X₁ is N, NH, or NR₁₀,
   wherein R₁₀ is alkyl, alkenyl, or alkynyl;
X₂ is C or N;
X₃ is CH or N; and
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H.

In some embodiments, the selective RBP4 antagonist is the compound wherein B or B₂ has the structure

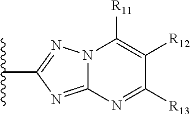

wherein
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein B or B₂ has the structure

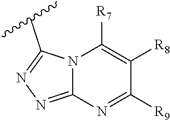

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein B or B₂ has the structure

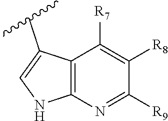

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃), C(O)—NHCH₃, NHC(O)—N(CH₃)₂, CN, or CF₃.

In some embodiments, the selective RBP4 antagonist is the compound wherein B or B₂ has the structure

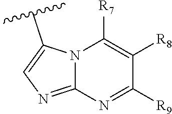

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein B or B₂ has the structure:

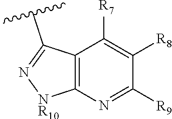

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃; and
R₁₀ is alkyl, alkenyl, or alkynyl.

In some embodiments, the selective RBP4 antagonist is the compound wherein R₇, R₈, and R₉ are each independently H, Cl, Br, F, OCH₃, OCH₂CH₃, CF₃, CN, CH₃, CH₂CH₃, C(O)OH, or C(O)—NH₂.

In some embodiments, the selective RBP4 antagonist is the compound wherein R₇, R₈, and R₉ are each independently H, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein R₇, R₈, and R₉ are each independently H, halogen, or alkyl.

In some embodiments, the selective RBP4 antagonist is the compound wherein two of R₇, R₈, and R₉ are each H and the remaining one of R₇, R₈, and R₉ is other than H.

In some embodiments, the selective RBP4 antagonist is the compound wherein one of R₇, R₈, and R₉ is H and the remaining two of R₇, R₈, and R₉ are each other than H.

In some embodiments, the selective RBP4 antagonist is the compound wherein B or B₂ has the structure

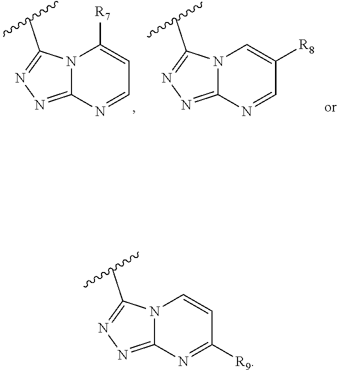

In some embodiments, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

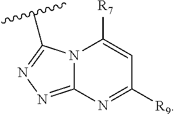

In some embodiments, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

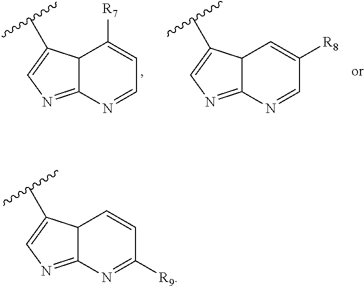

In some embodiments, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

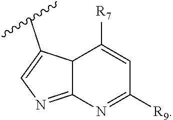

In some embodiments, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B has the structure

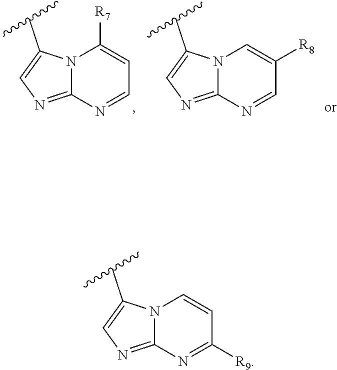

In some embodiments, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

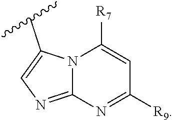

In some embodiments, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

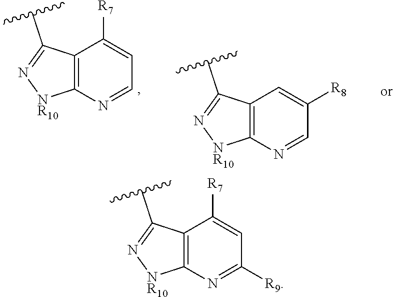

In some embodiments, the selective RBP4 antagonist is the compound wherein:
R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br; and
R₁₀ is alkyl.

In some embodiments, the selective RBP4 antagonist is the compound wherein B or B₂ has the structure

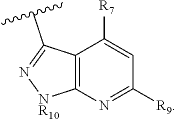

In some embodiments, the selective RBP4 antagonist is the compound wherein:
R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br; and
R₁₀ is alkyl.

In some embodiments, the selective RBP4 antagonist is the compound wherein B or B₂ has the structure

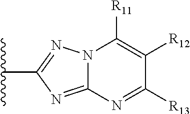

wherein R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein R₁₁, R₁₂, and R₁₃ are each independently H, Cl, Br, F, OCH₃, OCH₂CH₃, CF₃, CN, CH₃, CH₂CH₃, C(O)OH, or C(O)—NH₂.

In some embodiments, the selective RBP4 antagonist is the compound wherein R₁₁, R₁₂, and R₁₃ are each independently H, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein R₁₁, R₁₂, and R₁₃ are each independently H, halogen, or alkyl.

In some embodiments, the selective RBP4 antagonist is the compound wherein two of R₁₁, R₁₂, and R₁₃ are each H and the remaining one of R₁₁, R₁₂, and R₁₃ is other than H.

In some embodiments, the selective RBP4 antagonist is the compound wherein one of R₁₁, R₁₂, and R₁₃ is H and the remaining two of R₁₁, R₁₂, and R₁₃ are each other than H.

In some embodiments, the selective RBP4 antagonist is the compound wherein B or B₂ has the structure

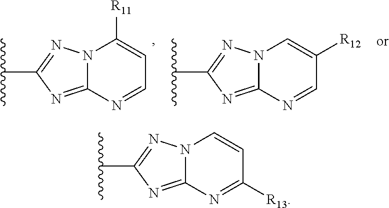

In some embodiments, the selective RBP4 antagonist is the compound wherein R₁₁, R₁₂, and R₁₃ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

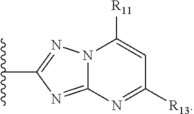

In some embodiments, the selective RBP4 antagonist is the compound wherein R₁₁ and R₁₃ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein X is N.

In some embodiments, the selective RBP4 antagonist is the compound wherein X is CH.

In some embodiments, the selective RBP4 antagonist is the compound wherein R₁, R₂, R₃, R₄, and R₅ are each H, t-Bu, Cl, F, or CF₃.

In some embodiments, the selective RBP4 antagonist is the compound wherein:
R₁, R₂, R₃, and R₄ are each H; and
R₅ is CF₃ or t-Bu.

In some embodiments, the selective RBP4 antagonist is the compound wherein:
R₁, R₃, and R₄ are each H;
R₂ is halogen; and
R₅ is CF₃ or t-Bu.

In some embodiments, the selective RBP4 antagonist is the compound wherein:
R₁, R₂, R₃, and R₄ are each H; and
R₅ is CF₃ or t-Bu.

In some embodiments, the selective RBP4 antagonist is the compound wherein:
R₁, R₂, R₃, and R₄ are each H; and
R₅ is CF₃.

In some embodiments, the selective RBP4 antagonist is the compound wherein one of R₁, R₂, R₃, R₄, and R₅ is other than H.

In some embodiments, the selective RBP4 antagonist is the compound wherein two of R₁, R₂, R₃, R₄, and R₅ are other than H.

In some embodiments, the selective RBP4 antagonist is the compound wherein two or more of R₁, R₂, R₃, R₄, and R₅ are other than H.

In some embodiments, the selective RBP4 antagonist is the compound wherein three of R₁, R₂, R₃, R₄, and R₅ are other than H.

In some embodiments, the selective RBP4 antagonist is the compound wherein three or more of R₁, R₂, R₃, R₄, and R₅ are other than H.

In some embodiments, the selective RBP4 antagonist is

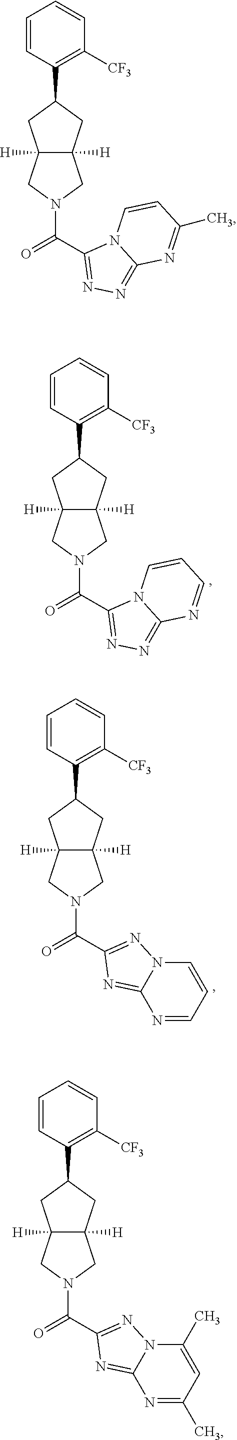 [SMILES: Cc1ccn2c(C(=O)N3C[C@H]4C[C@@H](c5ccccc5C(F)(F)F)C[C@H]4C3)nnc2n1 ; O=C(c1nnc2ncccn12)N1C[C@H]2C[C@@H](c3ccccc3C(F)(F)F)C[C@H]2C1 ; O=C(c1nc2ncccn2n1)N1C[C@H]2C[C@@H](c3ccccc3C(F)(F)F)C[C@H]2C1 ; Cc1cc(C)n2nc(C(=O)N3C[C@H]4C[C@@H](c5ccccc5C(F)(F)F)C[C@H]4C3)nc2n1]

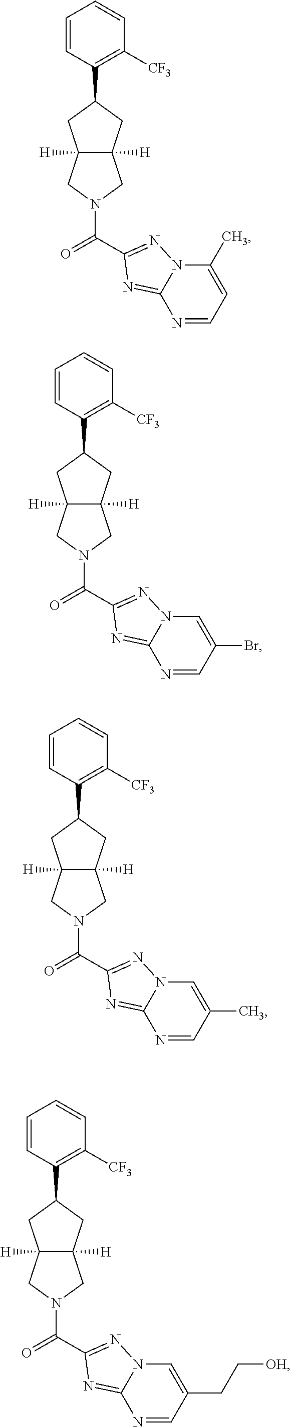 [SMILES: Cc1ccnc2nc(C(=O)N3C[C@H]4C[C@@H](c5ccccc5C(F)(F)F)C[C@H]4C3)nn12 ; O=C(c1nc2ncc(Br)cn2n1)N1C[C@H]2C[C@@H](c3ccccc3C(F)(F)F)C[C@H]2C1 ; Cc1cnc2nc(C(=O)N3C[C@H]4C[C@@H](c5ccccc5C(F)(F)F)C[C@H]4C3)nn2c1 ; O=C(c1nc2ncc(CCO)cn2n1)N1C[C@H]2C[C@@H](c3ccccc3C(F)(F)F)C[C@H]2C1]

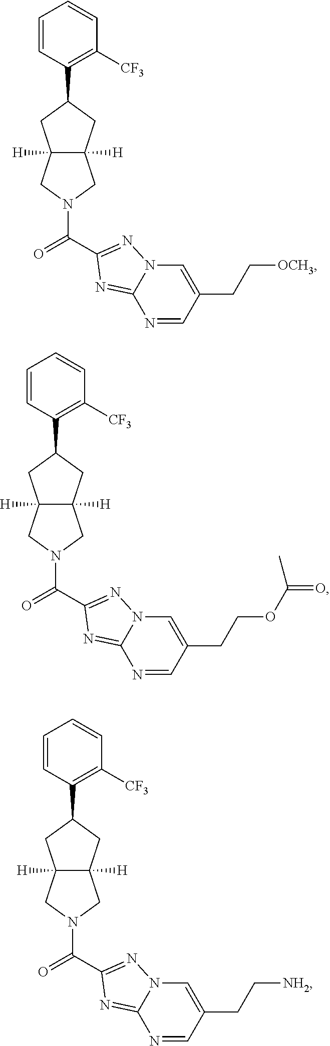 [SMILES: COCCc1cnc2nc(C(=O)N3C[C@H]4C[C@@H](c5ccccc5C(F)(F)F)C[C@H]4C3)nn2c1 ; CC(=O)OCCc1cnc2nc(C(=O)N3C[C@H]4C[C@@H](c5ccccc5C(F)(F)F)C[C@H]4C3)nn2c1 ; NCCc1cnc2nc(C(=O)N3C[C@H]4C[C@@H](c5ccccc5C(F)(F)F)C[C@H]4C3)nn2c1]

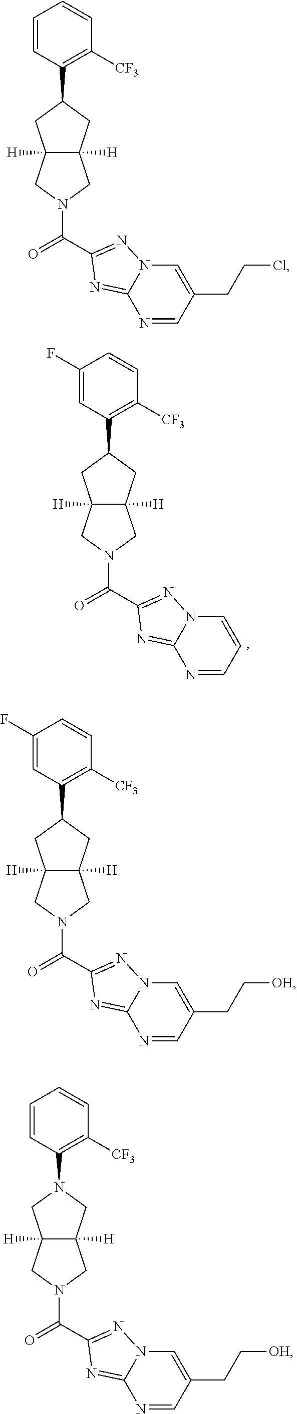

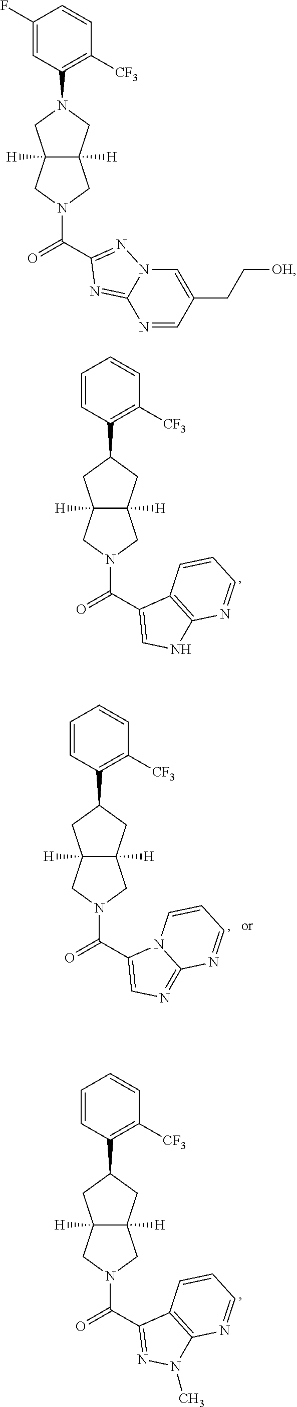

or a pharmaceutically acceptable salt thereof.

In some embodiments, the selective RBP4 antagonist is the compound wherein B or B₂ is other than

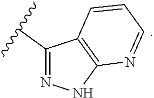 [SMILES: *c1n[nH]c2ncccc12]

In some embodiments, the selective RBP4 antagonist is the compound having the structure:

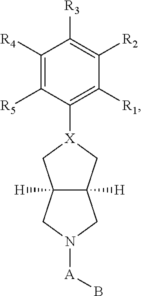

wherein
R₁, R₂, R₃, R₄, and R₅ are each independently H, halogen, alkyl, haloalkyl, O-haloalkyl, aryl, or heteroaryl;
X is N or CR₆,
   wherein R₆ is H, OH, or halogen;

A is absent or present, and when present, is or

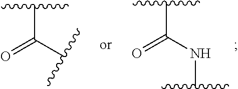

and
B has the structure:

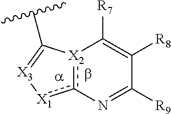

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

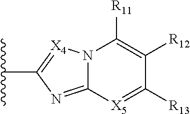

wherein
X₄ and X₅ are each independently N or CH; and
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound having the structure:

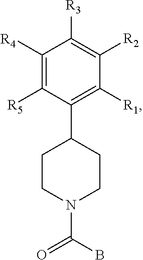

wherein
R₁, R₂, R₃, R₄, and R₅ are each independently H, halogen, CF₃, C₁-C₄ alkyl, aryl, or heteroaryl; and
B has the structure:

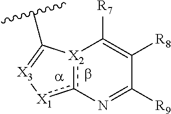

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

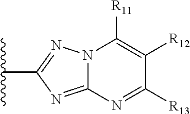

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound having the structure:

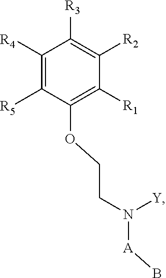

wherein
R₁, R₂, R₃, R₄, and R₅ are each independently H, halogen, CF₃, C₁-C₄ alkyl, aryl, or heteroaryl;
Y is alkyl;
A is absent or present, and when present, is or

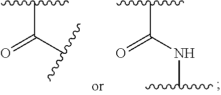

and
B has the structure:

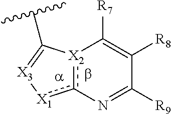

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

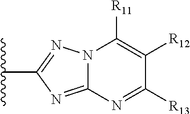

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound wherein one of R₁, R₂, R₃, R₄, and R₅ is other than H.

In some embodiments, the selective RBP4 antagonist is the compound wherein two of R₁, R₂, R₃, R₄, and R₅ are other than H.

In some embodiments, the selective RBP4 antagonist is the compound wherein R₁, R₂, R₃, R₄, and R₅ are each H, methyl, ethyl, phenyl, t-Bu, i-Pr, OCF₃, CF₃, OCF₂CF₃, CF₂CF₃, Cl, Br, or F.

In some embodiments, the selective RBP4 antagonist is the compound wherein:
R₁, R₂, R₃, and R₄ are each H; and
R₅ is —H, OCF₃, CF₂CF₃, methyl, ethyl, i-Pr, or phenyl.

In some embodiments, the selective RPB4 antagonist is

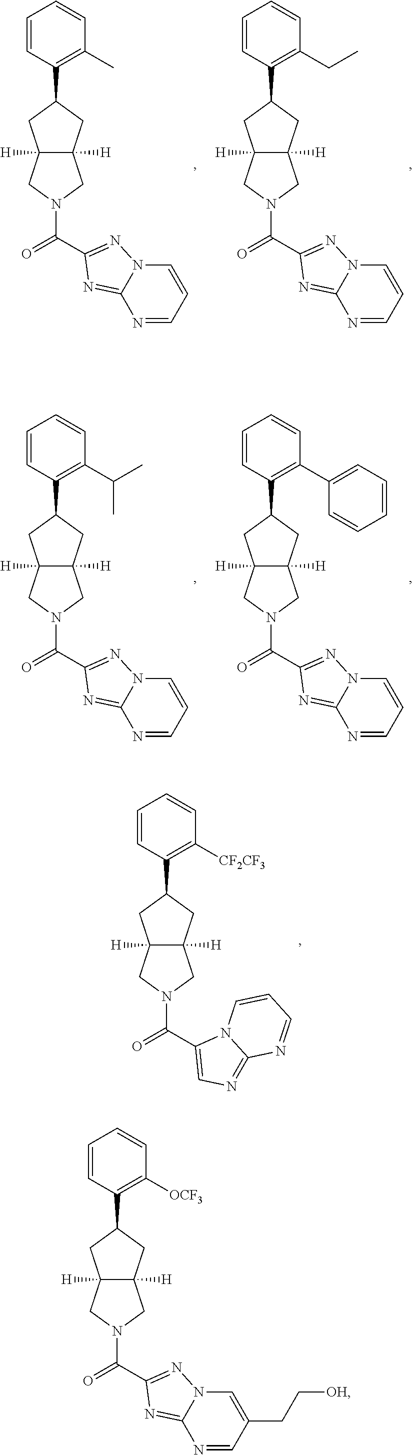

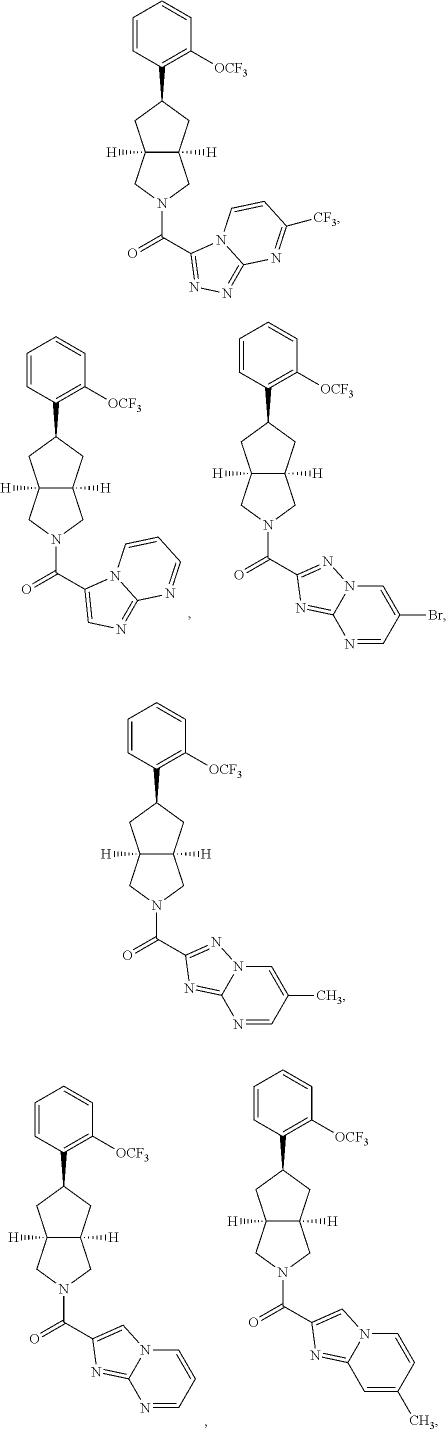 [SMILES: O=C(c1nnc2nc(C(F)(F)F)ccn12)N1C[C@H]2C[C@@H](c3ccccc3OC(F)(F)F)C[C@H]2C1 ; O=C(c1cnc2ncccn12)N1C[C@H]2C[C@@H](c3ccccc3OC(F)(F)F)C[C@H]2C1 ; O=C(c1nc2ncc(Br)cn2n1)N1C[C@H]2C[C@@H](c3ccccc3OC(F)(F)F)C[C@H]2C1 ; Cc1cnc2nc(C(=O)N3C[C@H]4C[C@@H](c5ccccc5OC(F)(F)F)C[C@H]4C3)nn2c1 ; O=C(c1cn2cccnc2n1)N1C[C@H]2C[C@@H](c3ccccc3OC(F)(F)F)C[C@H]2C1 ; Cc1ccn2cc(C(=O)N3C[C@H]4C[C@@H](c5ccccc5OC(F)(F)F)C[C@H]4C3)nc2c1]

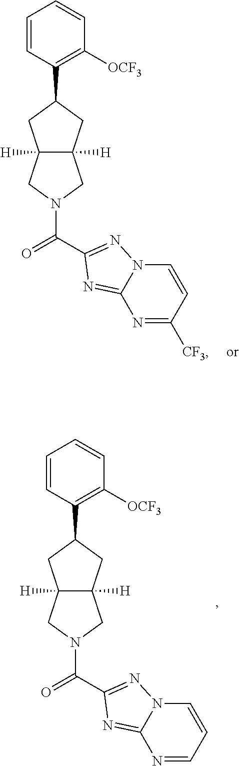 [SMILES: O=C(c1nc2nc(C(F)(F)F)ccn2n1)N1C[C@H]2C[C@@H](c3ccccc3OC(F)(F)F)C[C@H]2C1 ; O=C(c1nc2ncccn2n1)N1C[C@H]2C[C@@H](c3ccccc3OC(F)(F)F)C[C@H]2C1]

or a pharmaceutically acceptable salt thereof.

In some embodiments, a is 2 and b is 1.

In some embodiments, a is 1 and b is 1.

In some embodiments, a is 2, b is 1, and M is

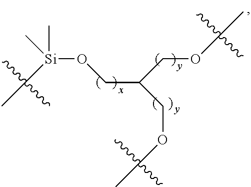

wherein x is 1, 2, 3, 4, or 5, and y is 1, 2, 3, 4, or 5.

In some embodiments, a is 2, b is 1, and M is

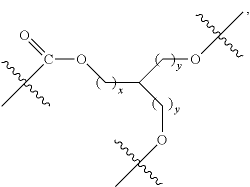

wherein x is 1, 2, 3, 4, or 5, and y is 1, 2, 3, 4, or 5.

In some embodiments, a is 2, b is 1, and M is

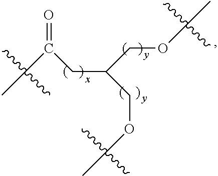

wherein x is 0, 1, 2, 3, or 4, and y is 1, 2, 3, 4, or 5.

In some embodiments, a is 1, b is 1, and M is

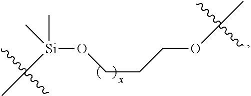

wherein x is 1, 2, 3, 4, or 5.

In some embodiments, a is 1, b is 1, and M is

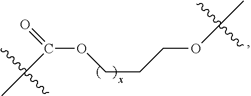

wherein x is 1, 2, 3, 4, or 5.

In some embodiments, a is 1, b is 1, and M is

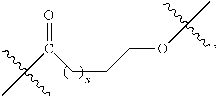

wherein x is 0, 1, 2, 3, or 4.

The present disclosure provides a pharmaceutical composition comprising the compound (P-)ₐM(-Q)_{b} or P(-Q)_{b} of the present disclosure and a pharmaceutically acceptable carrier.

The present disclosure provides a method for treating a disease characterized by excessive or age-related lipofuscin accumulation in the retina in a mammal afflicted therewith, and for promoting rhodopsin and cone opsins production, comprising administering to the mammal an effective amount of the compound of the present disclosure or an effective amount of the pharmaceutical composition of the present disclosure.

In some embodiments of the method, the disease is further characterized by bisretinoid-mediated macular degeneration.

In some embodiments of the method, the amount of the compound is effective to lower the serum concentration of RBP4 in the mammal, or the amount of the compound is effective to lower the retinal concentration of a bisretinoid in lipofuscin in the mammal.

In some embodiments of the method, the bisretinoid is A2E.

In some embodiments of the method, the bisretinoid is isoA2E.

In some embodiments of the method, the bisretinoid is A2-DHP-PE.

In some embodiments of the method, the bisretinoid is atRAL di-PE.

In some embodiments of the method, the disease characterized by age-related lipofuscin accumulation in the retina is Age-Related Macular Degeneration.

In some embodiments of the method, the disease characterized by age-related lipofuscin accumulation in the retina is dry (atrophic) Age-Related Macular Degeneration.

In some embodiments of the method, the disease characterized by excessive lipofuscin accumulation in the retina is Stargardt Disease.

In some embodiments of the method, the disease characterized by excessive lipofuscin accumulation in the retina is other forms of retinopathy caused by or associated with mutations in the ABCA4 gene, such as retinitis pigmentosa (RP19) or cone-rod dystrophy (CORD3).

In some embodiments of the method, the disease characterized by excessive lipofuscin accumulation in the retina is Best disease.

In some embodiments of the method, the disease characterized by excessive lipofuscin accumulation in the retina is adult vitelliform maculopathy.

In some embodiments of the method, the disease characterized by excessive lipofuscin accumulation in the retina is Stargardt-like macular dystrophy.

In some embodiments of the method, the administration is effective to reduce photoreceptor degeneration.

In some embodiments of the method, the bisretinoid-mediated macular degeneration is Age-Related Macular Degeneration or Stargardt Disease.

In some embodiments of the method, the bisretinoid-mediated macular degeneration is Age-Related Macular Degeneration.

In some embodiments of the method, the bisretinoid-mediated macular degeneration is dry (atrophic) Age-Related Macular Degeneration.

In some embodiments of the method, the bisretinoid-mediated macular degeneration is Stargardt Disease.

In some embodiments of the method, the bisretinoid-mediated macular degeneration is other forms of retinopathy caused by or associated with mutations in the ABCA4 gene, such as retinitis pigmentosa (RP19) or cone-rod dystrophy (CORD3).

In some embodiments of the method, the bisretinoid-mediated macular degeneration is Best disease.

In some embodiments of the method, the bisretinoid-mediated macular degeneration is adult vitelliform maculopathy.

In some embodiments of the method, the bisretinoid-mediated macular degeneration is Stargardt-like macular dystrophy.

In some embodiments, the compound of the present disclosure, upon hydrolysis and/or proteolytic cleavage, exhibits retinol-binding protein 4 (RBP4) antagonist activity as well as activity of promoting rhodopsin and cone opsins production.

In some embodiments, the compound of the present disclosure, upon hydrolysis and/or proteolytic cleavage, reduces circulating RBP4 levels and promotes rhodopsin and cone opsins production.

In some embodiments, the compound of the present disclosure or the pharmaceutical composition of the present disclosure may be used for the treatment of dry age-related macular degeneration (AMD).

In some embodiments, the compound of the present disclosure or the pharmaceutical composition of the present disclosure may be used for the treatment of type 2 diabetes.

In some embodiments, the compound of the present disclosure or the pharmaceutical composition of the present disclosure may be used for the treatment of obesity.

In some embodiments, the compound of the present disclosure or the pharmaceutical composition of the present disclosure may be used for the treatment of insulin resistance.

In some embodiments, the compound of the present disclosure or the pharmaceutical composition of the present disclosure may be used for the treatment of cardiovascular disease.

In some embodiments, the compound of the present disclosure or the pharmaceutical composition of the present disclosure may be used for the treatment of hepatic steatosis.

In some embodiments, the compound of the present disclosure or the pharmaceutical composition of the present disclosure may be used for the treatment of non-alcoholic fatty liver disease (NAFLD).

The present disclosure provides a method for treating a non-alcoholic fatty liver disease (NAFLD) in a mammal afflicted therewith, and for promoting rhodopsin and cone opsins production, comprising administering to the mammal an effective amount of the compound of the present disclosure or an effective amount of the pharmaceutical composition of the present disclosure.

The present disclosure provides a method for treating gout in a mammal afflicted therewith, and for promoting rhodopsin and cone opsins production, comprising administering to the mammal an effective amount of the compound of the present disclosure or an effective amount of the pharmaceutical composition of the present disclosure.

In some embodiments of the method, the mammal is afflicted with a NAFLD selected from the group consisting of hepatic steatosis (fatty liver), non-alcoholic steatohepatitis (NASH), cirrhosis, and hepatocellular carcinoma.

In some embodiments of the method, the method further comprises a step of determining, or having determined, the level of RBP4 in adipose tissue in the mammal and administering to the mammal the compound of the present disclosure or the pharmaceutical composition of the present disclosure if the level of RBP4 in adipose tissue is elevated.

In some embodiments of the method, the method further comprises a step of determining, or having determined, the level of RBP4 in serum in the mammal and administering to the mammal the compound of the present disclosure or the pharmaceutical composition of the present disclosure if the level of RBP4 in serum is elevated.

In some embodiments of the method, the amount of the compound of the present disclosure or the amount of the pharmaceutical composition of the present disclosure is effective in reducing RBP4 levels in adipose tissue in the mammal.

In some embodiments of the method, the amount of the compound of the present disclosure or the amount of the pharmaceutical composition of the present disclosure is effective in reducing RBP4 levels in serum in the mammal.

In some embodiments of the method, the amount of the compound of the present disclosure or the amount of the pharmaceutical composition of the present disclosure is effective in reducing uric acid levels in the serum of the mammal.

In some embodiments of the method, the amount of the compound of the present disclosure or the amount of the pharmaceutical composition of the present disclosure is effective to normalize the concentration of triglycerides in the liver of the mammal.

In some embodiments of the method, the amount of the compound of the present disclosure or the amount of the pharmaceutical composition of the present disclosure is effective to normalize the concentration of free fatty acids in the serum of the mammal.

In some embodiments of the method, the amount of the compound of the present disclosure or the amount of the pharmaceutical composition of the present disclosure is effective to normalize the concentration of free fatty acids in the liver of the mammal.

In some embodiments of the method, the amount of the compound of the present disclosure or the amount of the pharmaceutical composition of the present disclosure is effective to prevent trafficking of a fatty acid by RBP4.

In some embodiments of the method, the amount of the compound of the present disclosure or the amount of the pharmaceutical composition of the present disclosure is effective to prevent trafficking of a fatty acid to the liver by RBP4.

In some embodiments of the method, the amount of the compound of the present disclosure or the amount of the pharmaceutical composition of the present disclosure is effective to inhibit binding between RBP4 and a fatty acid.

In some embodiments of the method, the fatty acid is from adipose tissue.

In some embodiments of the method, the mammal does not have elevated serum RBP4 levels.

In some embodiments of the method, the mammal has elevated serum RBP4 levels.

In some embodiments of the method, the serum RBP4 level of the mammal is elevated by more than 3 micrograms per ml.

In some embodiments of the method, the NAFLD is a hepatic steatosis selected from simple hepatic steatosis and mild hepatic steatosis.

In some embodiments of the method, the amount of the compound of the present disclosure administered is such that the amount of the selective RBP4 antagonist released in the body of the mammal is 5-1000 mg, 5-800 mg, 5-200 mg, 45-200 mg, 45-1000 mg, 45-800 mg, 10-50 mg, 96 mg, 24 mg, or 10 mg per day.

In some embodiments of the method, the method further comprises administering an amount of a second agent which is (R)-(+)-(5,6-dichloro 2,3,9,9a-tetrahydro 3-oxo-9a-propyl-1H-fluoren-7-yl)oxy]acetic acid (DPOFA), a Nonsteroidal Anti-inflammatory Drug (NSAID) such as indomethacin, colchicine, lesinurad, corticosteroids (e.g., betamethasone, prednisone, dexamethasone, cortisone, hydrocortisone, methylprednisone, prednisolone), biologic anti-IL-1alpha/beta agents (e.g., canakinumab, rilonacept, anakinra), allopurinol, benzbromarone, pegloticase, and other forms of uricase enzymes, topiroxostat (FYX-051), ulodesine (BCX4208), KUX-1151, RLBN1001, RDEA3170, arhalofenate (MBX-102), levotofisopam, UR-1102, PF-06743649, BCX4208, SHR4640, Lumiracoxib, Tranilast, Topiroxostat, LC350189, Bucillamine, AC-201, HuZhen Capsules (including Polygonum cuspidatum and Ligustrum lucidum), MPC-004, FYU-981, Sodium Bicarbonate, SEL-212, SEL-037, Apremilast, TMX-67, SSS11, D-0120, febuxostat, or probenecid, or esters or salts thereof, effective to treat the mammal.

In some embodiments of the method, the second agent is DPOFA.

In some embodiments of the method, the mammal is afflicted with gout.

In some embodiments of the method, the amount of the second agent and/or the amount of the compound or the pharmaceutical composition of the present disclosure is effective in reducing uric acid levels in the blood of the mammal.

In some embodiments of the method, the amount of the second agent and/or the amount of the compound or the pharmaceutical composition of the present disclosure is effective in decreasing uric acid reabsorption in the kidneys of the mammal.

In some embodiments of the method, the amount of the second agent is effective in increasing uric acid clearance in the mammal.

In some embodiments of the method, the amount of the second agent is effective in increasing uric acid levels in the urine of the mammal.

In some embodiments of the method, the amount of the second agent is effective in increasing renal clearance of uric acid in the mammal.

In some embodiments of the method, the amount of the second agent and/or the amount of the compound or the pharmaceutical composition of the present disclosure is effective in reducing one or more symptoms associated with gout in the mammal.

In some embodiments of the method, the one or more symptoms associated with gout are selected from joint pain, joint inflammation, joint redness, and decreased range of motion at the joint.

In some embodiments of the method, the amount of the second agent and/or the amount of the compound or the pharmaceutical composition of the present disclosure is effective in preventing gout in the mammal.

In some embodiments of the method, the preventing comprises increasing uric acid levels in the urine of the mammal.

In some embodiments of the method, the preventing comprises reducing uric acid levels in the blood of the mammal.

In some embodiments of the method, the preventing comprises increasing uric acid clearance in the mammal.

In some embodiments of the method, the preventing comprises decreasing uric acid reabsorption in the kidneys of the mammal.

In some embodiments of the method, the preventing comprises increasing renal clearance of uric acid in the mammal.

In some embodiments of the method, the preventing comprises reducing one or more symptoms associated with gout in the mammal.

In some embodiments of the method, the one or more symptoms associated with gout are selected from joint pain, joint inflammation, joint redness, and decreased range of motion at the joint.

In some embodiments of the method, the gout is chronic gout.

In some embodiments of the method, the gout is acute gout.

In some embodiments of the method, the amount of the second agent and/or the amount of the compound or the pharmaceutical composition of the present disclosure prevents a recurrence of chronic gout.

In some embodiments of the method, the mammal is female and the administration of the compound of the present disclosure or the administration of the pharmaceutical composition of the present disclosure reduces the uric acid level to 2.4-6.0 mg/dL.

In some embodiments of the method, the mammal is male and the administration of the compound of the present disclosure or the administration of the pharmaceutical composition of the present disclosure reduces the uric acid level to 3.4-7.0 mg/dL.

In some embodiments of the method, the administration of the compound of the present disclosure or the administration of the pharmaceutical composition of the present disclosure reduces uric acid levels in the mammal to less than 7 mg/dL.

In some embodiments of the method, the compound or the pharmaceutical composition of the present disclosure and the second agent are administered sequentially, simultaneously, contemporaneously, or concomitantly, in which in a sequential administration, the compound or the pharmaceutical composition of the present disclosure may be administered first or the second agent may be administered first.

The present disclosure provides a pharmaceutical composition comprising the compound of the present disclosure for use in a combination therapy together with a pharmaceutical composition comprising the second agent, for the treatment of a non-alcoholic fatty liver disease (NAFLD) or gout.

The present disclosure provides a pharmaceutical composition comprising an amount of the compound of the present disclosure for use in treating a mammal afflicted with a non-alcoholic fatty liver disease (NAFLD) or gout as an add-on therapy to or in combination with the second agent.

In some embodiments of the method, the administration is oral.

In some embodiments, the mammal is a human.

In some embodiments, the C20-D3-visual-chromophore-producing compound is selected from the group consisting of C20-D3-retinol, C20-D3-9-cis-retinol, and C20-D3-11-cis-retinol.

The present disclosure provides a pharmaceutical composition comprising a selective RBP4 antagonist, a C20-D3-visual-chromophore-producing compound, and a pharmaceutically acceptable carrier, wherein:
the selective RBP4 antagonist is a compound having the structure:

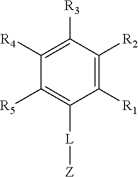

wherein L is a linking group having the structure
   and

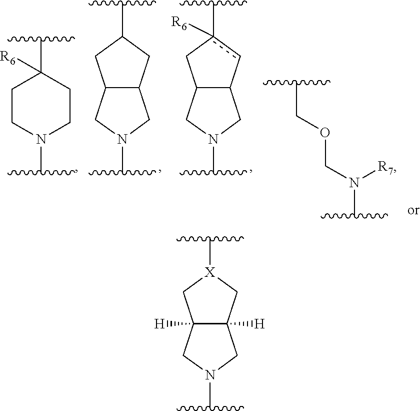

Z is a group having the structure

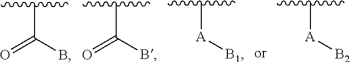

wherein
R₁, R₂, R₃, R₄, and R₅ are each independently H, halogen, CF₃, C₁-C₄ alkyl, aryl, or heteroaryl;
R₆ is H, OH, or halogen, or is absent;
ψ is absent or present, and when present, is a bond;
B is a substituted or unsubstituted heterobicycle, pyridazine,
pyrazole, pyrazine, thiadiazole, or triazole, wherein
the heterobicycle is other than chloro-substituted indole; and
the pyrazole, when substituted, is substituted with other than trifluoromethyl;
   B′ is a substituted or unsubstituted phenyl, pyridine, pyrimidine, benzyl, pyrrolidine, sulfolane, oxetane, CO₂H, or (C₁-C₄ alkylenyl)-CO₂H, wherein
the substituted phenyl is substituted with other than trifluoromethyl or 3-(methyl carboxylate),
the substituted pyridine is substituted with other than trifluoromethyl,
the substituted pyrrolidine is substituted with other than hydroxamic acid, and
the substituted or unsubstituted pyrrolidine is bound to the carbonyl through a carbon-carbon bond;
   A is absent or present, and when present, is

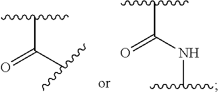

B₁ is a substituted or unsubstituted monocycle, bicycle, heteromonocycle, heterobicycle, benzyl, CO₂H, or (C₁-C₄ alkylenyl)-CO₂H,
wherein when B₁ is CO₂H, then A is present and is

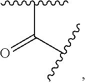 [SMILES: *C(*)=O]

R₇ is alkyl;

### X is N or CR₈,

wherein R₈ is H, OH, or halogen; and
   B₂ has the structure:

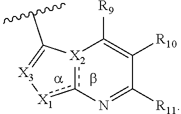

wherein
α and β are each a bond that is present or absent;
X₁ is N, NH, or NR₉₉,
wherein R₉₉ is alkyl, alkenyl, or alkynyl;

### X₂ is C or N;

### X₃ is CH or N;

R₉, R₁₀, and R₁₁ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃, wherein
X₁, X₂, and X₃ are each N, α is present, and β is absent; or
X₁ is NH, X₂ is C, X₃ is CH, α is absent, and β is present; or
X₁ is N, X₂ is N, X₃ is CH, α is present, and β is absent; or
X₁ is NH or NR₉₉, X₂ is C, X₃ is N, α is absent, and β is present, wherein when X₁ is NH, X₂ is C, X₃ is N, α is absent, and β is present, then one of R₉, R₁₀, and R₁₁ is other than H;
   or
   B₂ has the structure:

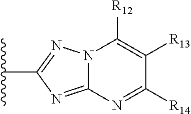

wherein
R₁₂, R₁₃, and R₁₄ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
or a pharmaceutically acceptable salt thereof; and
the C20-D3-visual-chromophore-producing compound is selected from the group consisting of C20-D3-retinol, C20-D3-retinaldehyde, C20-D3-retinyl esters, C20-D3-9-cis-retinol, C20-D3-9-cis-retinaldehye, C20-D3-9-cis-retinyl esters, C20-D3-11-cis-retinol, C20-D3-11-cis-retinaldehye, C20-D3-11-cis-retinyl esters, and C20-D3-C20′-D3-β-carotene.

In some embodiments, the selective RBP4 antagonist is the compound wherein R₁, R₂, R₃, R₄, and R₅ are each independently H, halogen, CF₃, or C₁-C₄ alkyl.

In some embodiments, the selective RPB4 antagonist is the compound wherein L is

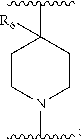

### and Z is

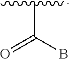

In some embodiments, the selective RBP4 antagonist is the compound wherein L is

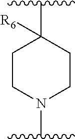

### and Z is

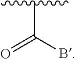

In some embodiments, the selective RBP4 antagonist is the compound wherein
L is

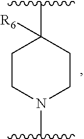

Z is

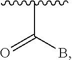

two or more of R₁, R₂, R₃, R₄, and R₅ are other than H, and
when
   R₁ is CF₃, R₂ is H, R₃ is F, R₄ is H, and R₅ is H, or
   R₁ is H, R₂ is CF₃, R₃ is H, R₄ is CF₃, and R₅ is H, or
   R₁ is Cl, R₂ is H, R₃ is H, R₄ is F, and R₅ is H, or
   R₁ is CF₃, R₂ is H, R₃ is F, R₄ is H, and R₅ is H, or
   R₁ is CF₃, R₂ is F, R₃ is H, R₄ is H, and R₅ is H, or
   R₁ is Cl, R₂ is F, R₃ is H, R₄ is H, and R₅ is H,
then B is other than

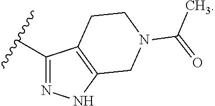 [SMILES: *c1n[nH]c2c1CCN(C(C)=O)C2]

In some embodiments, the selective RBP4 antagonist is the compound wherein L is

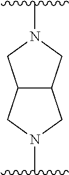 [SMILES: *N1CC2CN(*)CC2C1]

### and Z is

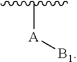

In some embodiments, the selective RBP4 antagonist is the compound wherein
L is

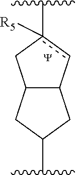

Z is

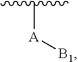

R₆ is absent or present, and when present, is H, OH, or halogen, and
when ψ is present, then R₆ is absent, and when ψ is absent, then R₆ is present.

In some embodiments, the selective RBP4 antagonist is the compound wherein L is

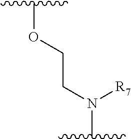

### and Z is

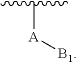

In some embodiments, the selective RBP4 antagonist is the compound wherein L is

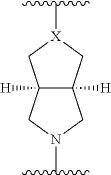

### and Z is

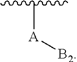

In some embodiments, the selective RBP4 antagonist is the compound wherein
L is

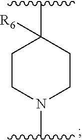

Z is

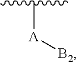

R₆ is H, and
A is

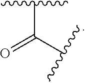 [SMILES: *C(*)=O]

In some embodiments, the selective RBP4 antagonist is the compound wherein L is

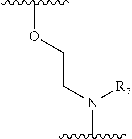

### and Z is

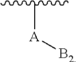

In some embodiments, the selective RBP4 antagonist is

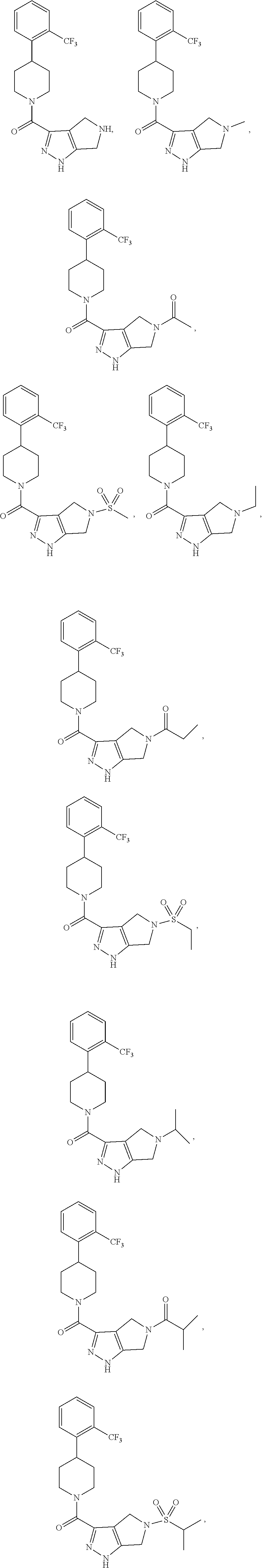

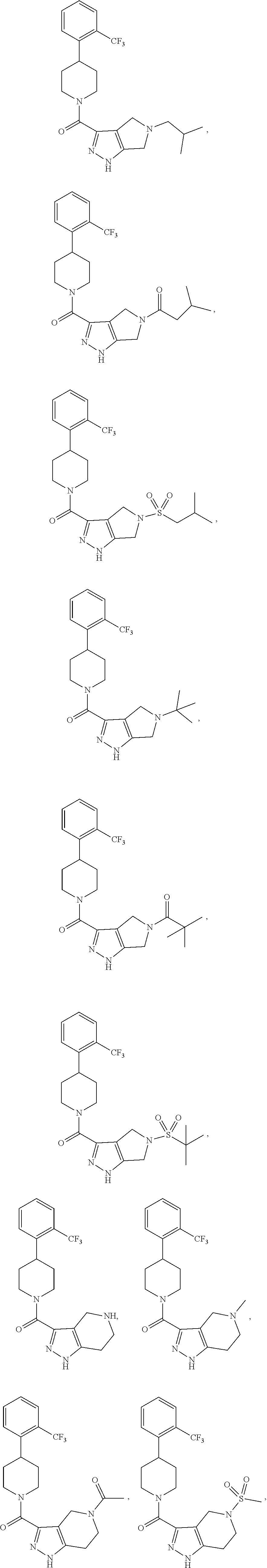

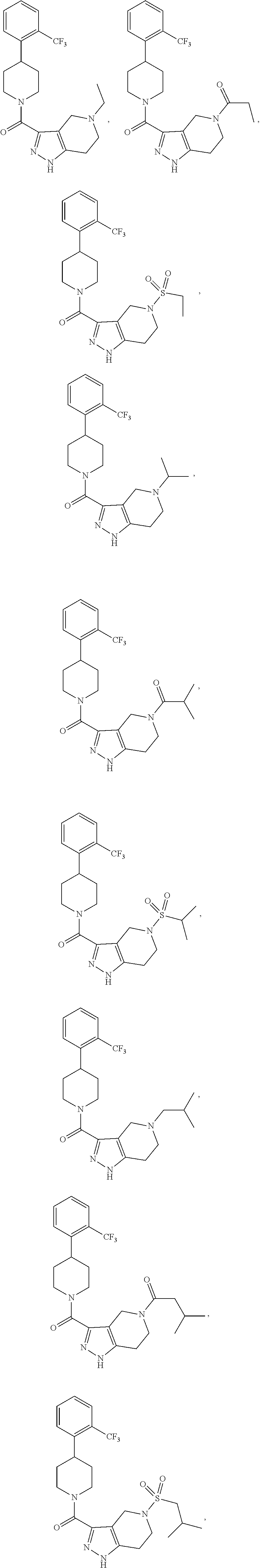

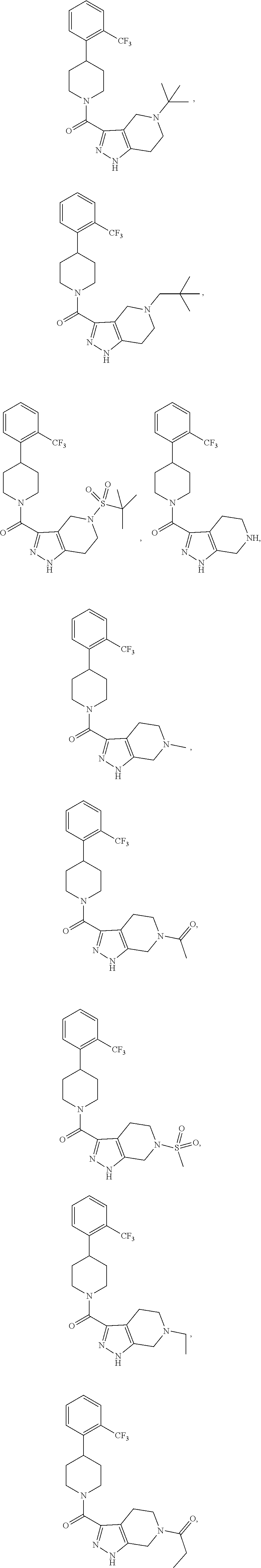

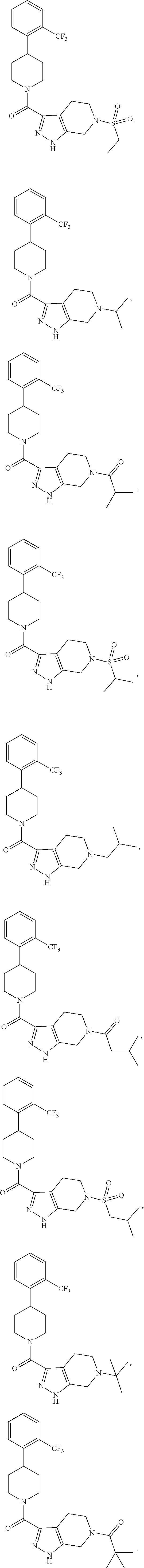

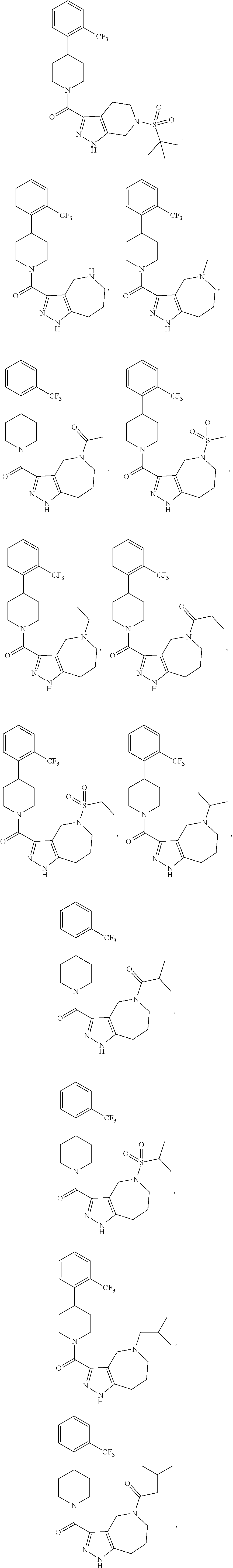

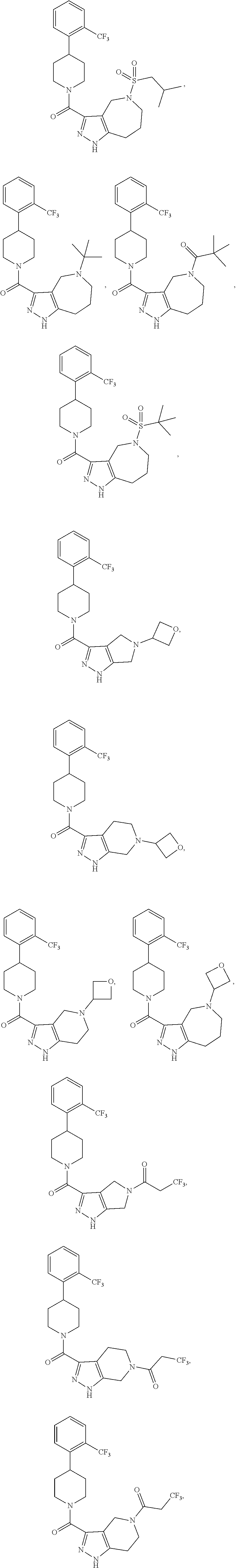

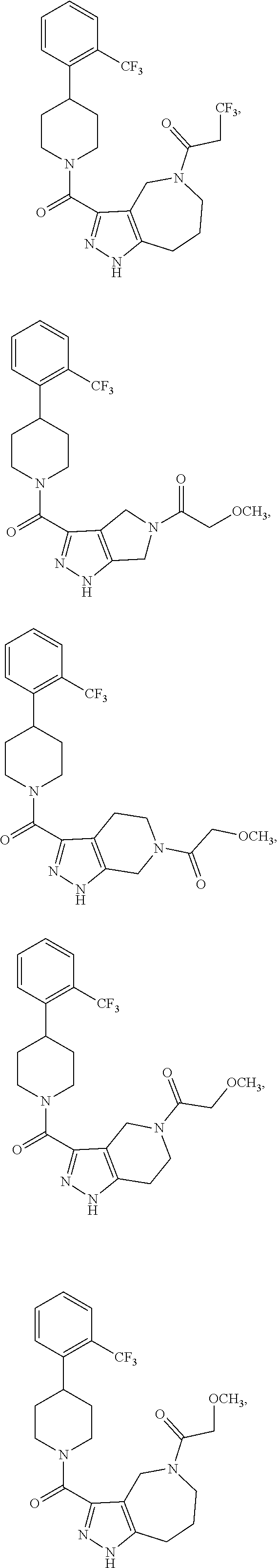 [SMILES: O=C(CC(F)(F)F)N1CCCc2[nH]nc(C(=O)N3CCC(c4ccccc4C(F)(F)F)CC3)c2C1 ; COCC(=O)N1Cc2[nH]nc(C(=O)N3CCC(c4ccccc4C(F)(F)F)CC3)c2C1 ; COCC(=O)N1CCc2c(C(=O)N3CCC(c4ccccc4C(F)(F)F)CC3)n[nH]c2C1 ; COCC(=O)N1CCc2[nH]nc(C(=O)N3CCC(c4ccccc4C(F)(F)F)CC3)c2C1 ; COCC(=O)N1CCCc2[nH]nc(C(=O)N3CCC(c4ccccc4C(F)(F)F)CC3)c2C1]

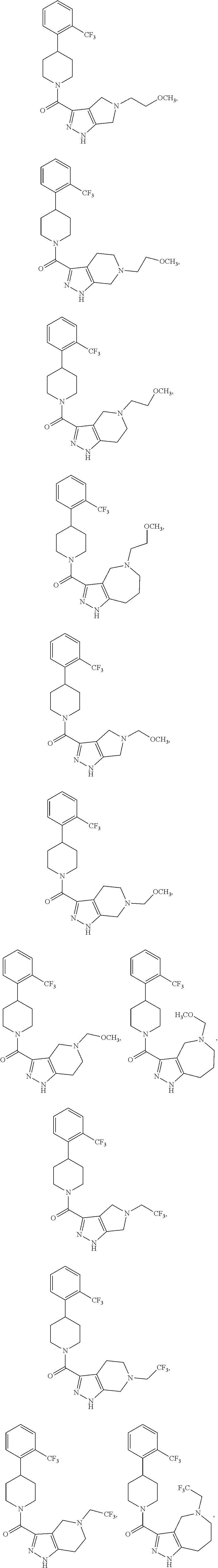

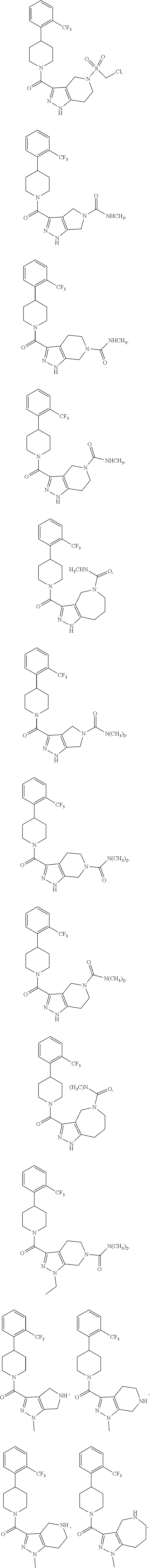

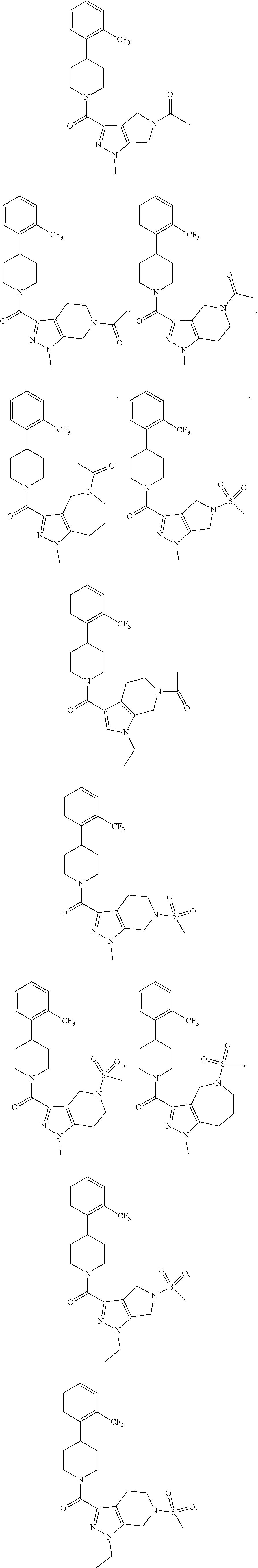

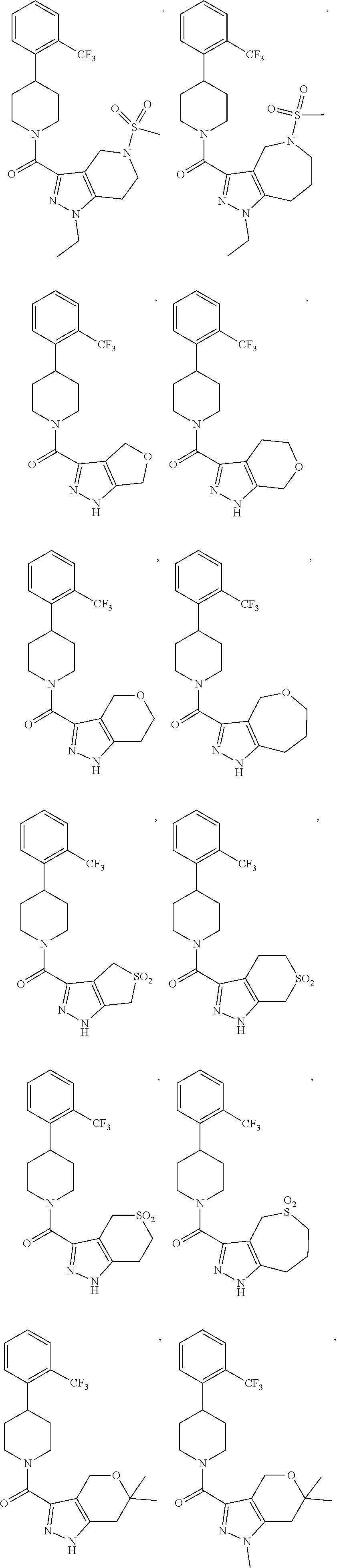

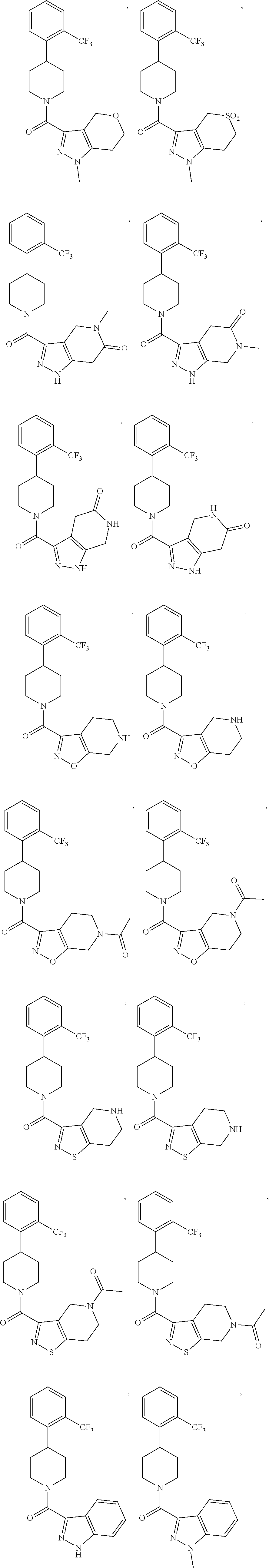

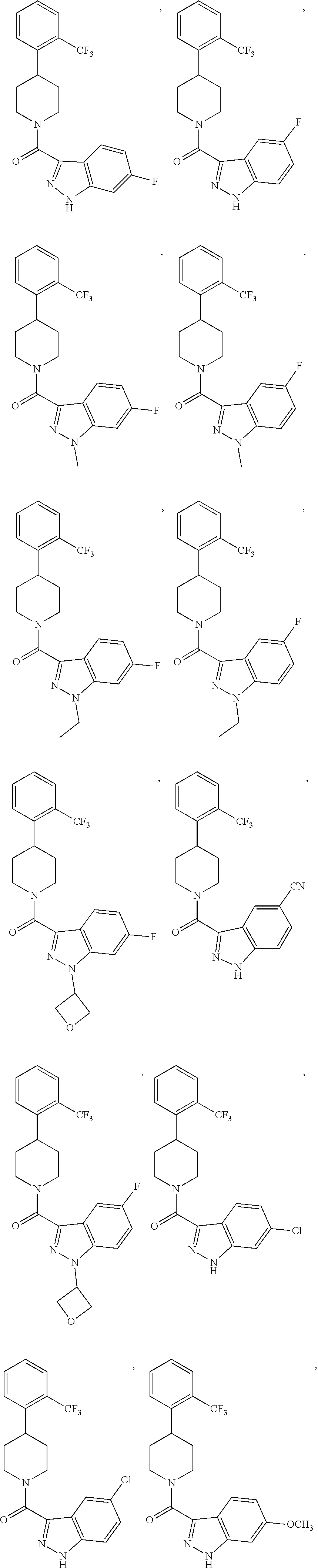

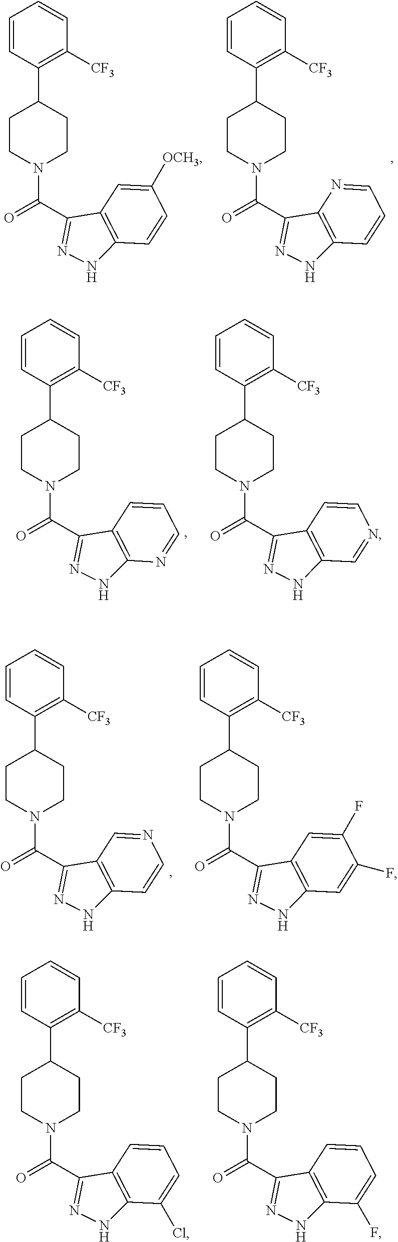

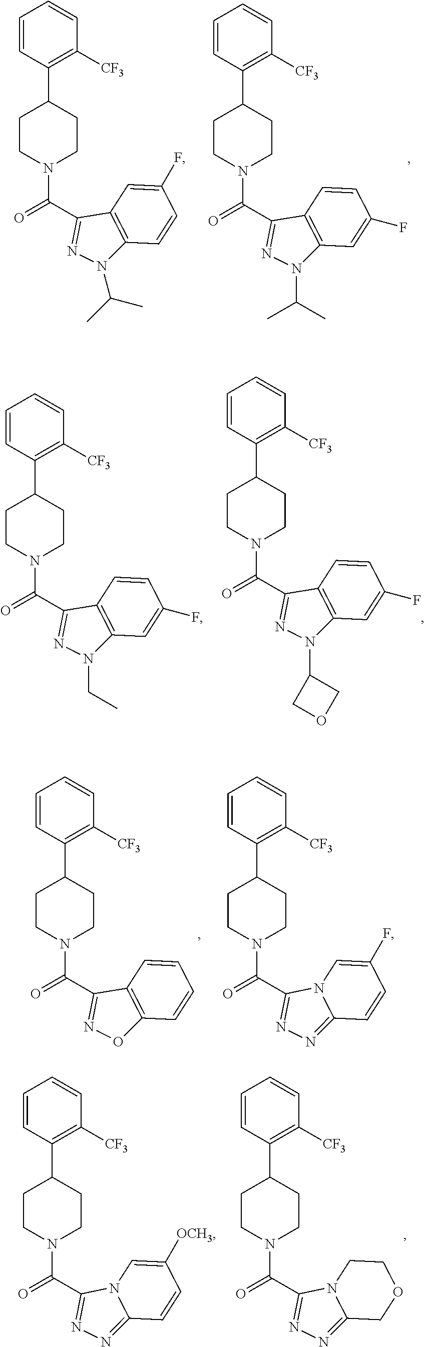

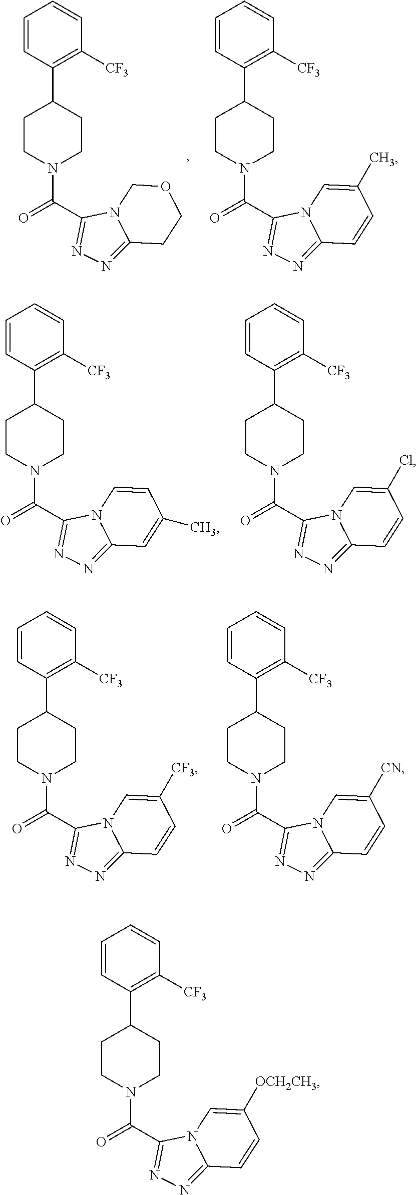

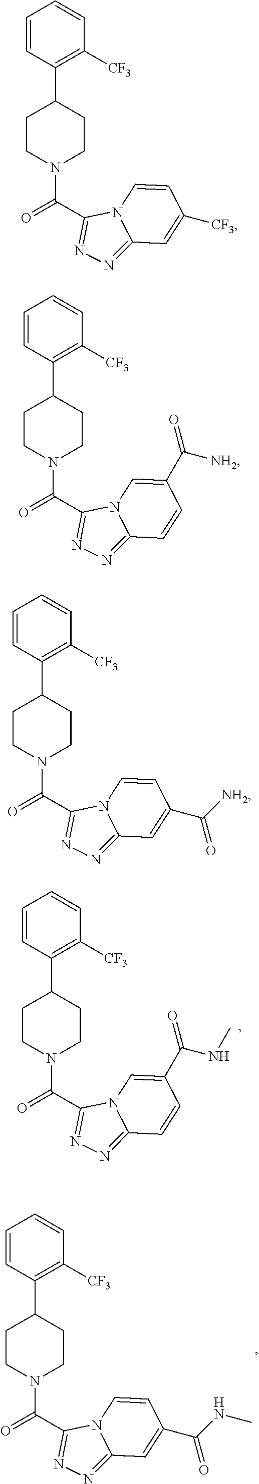 [SMILES: O=C(c1nnc2cc(C(F)(F)F)ccn12)N1CCC(c2ccccc2C(F)(F)F)CC1 ; NC(=O)c1ccc2nnc(C(=O)N3CCC(c4ccccc4C(F)(F)F)CC3)n2c1 ; NC(=O)c1ccn2c(C(=O)N3CCC(c4ccccc4C(F)(F)F)CC3)nnc2c1 ; CNC(=O)c1ccc2nnc(C(=O)N3CCC(c4ccccc4C(F)(F)F)CC3)n2c1 ; CNC(=O)c1ccn2c(C(=O)N3CCC(c4ccccc4C(F)(F)F)CC3)nnc2c1]

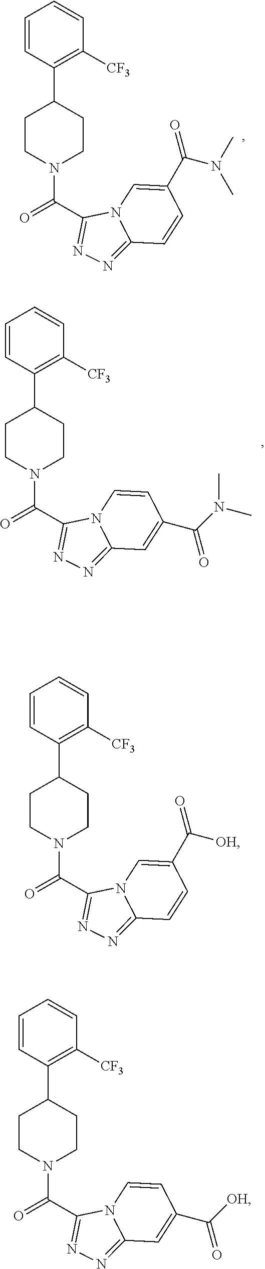 [SMILES: CN(C)C(=O)c1ccc2nnc(C(=O)N3CCC(c4ccccc4C(F)(F)F)CC3)n2c1 ; CN(C)C(=O)c1ccn2c(C(=O)N3CCC(c4ccccc4C(F)(F)F)CC3)nnc2c1 ; O=C(O)c1ccc2nnc(C(=O)N3CCC(c4ccccc4C(F)(F)F)CC3)n2c1 ; O=C(O)c1ccn2c(C(=O)N3CCC(c4ccccc4C(F)(F)F)CC3)nnc2c1]

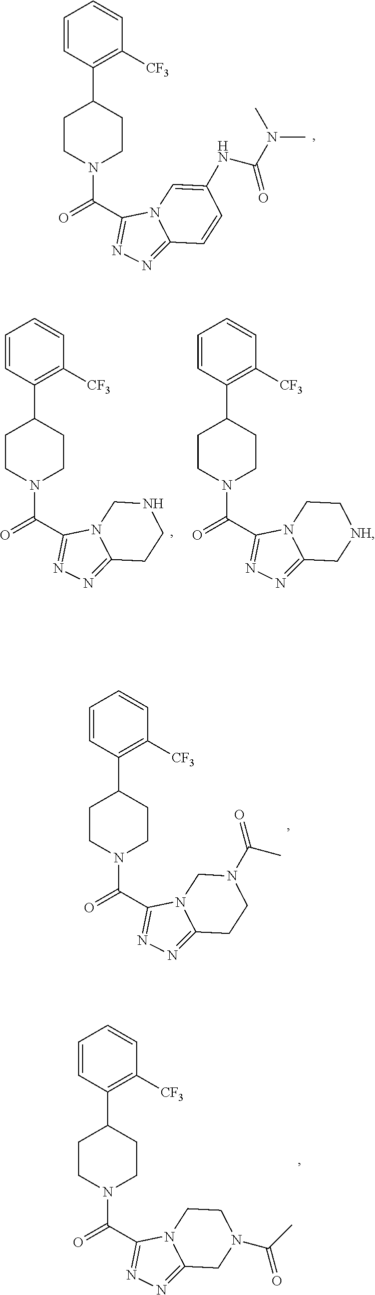

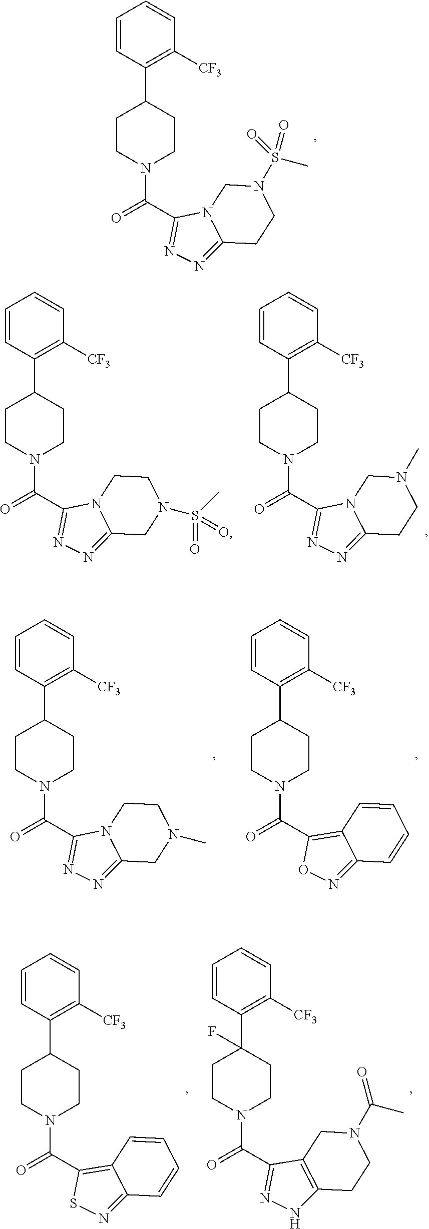

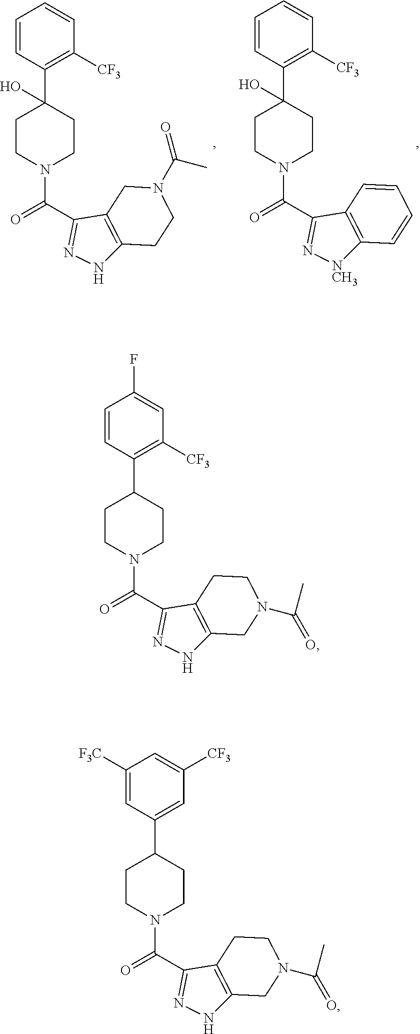 [SMILES: CC(=O)N1CCc2[nH]nc(C(=O)N3CCC(O)(c4ccccc4C(F)(F)F)CC3)c2C1 ; Cn1nc(C(=O)N2CCC(O)(c3ccccc3C(F)(F)F)CC2)c2ccccc21 ; CC(=O)N1CCc2c(C(=O)N3CCC(c4ccc(F)cc4C(F)(F)F)CC3)n[nH]c2C1 ; CC(=O)N1CCc2c(C(=O)N3CCC(c4cc(C(F)(F)F)cc(C(F)(F)F)c4)CC3)n[nH]c2C1]

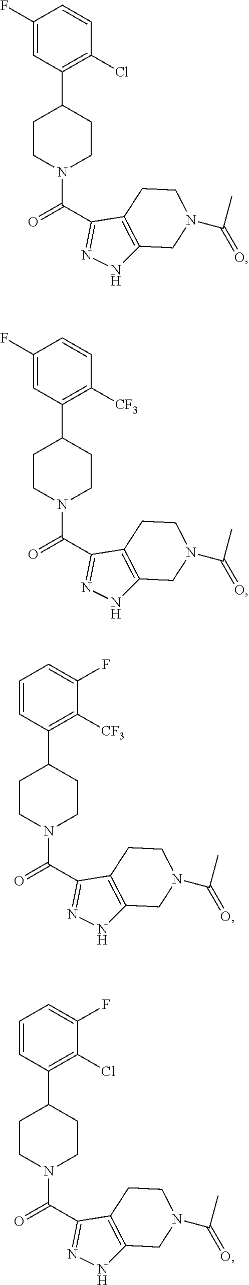 [SMILES: CC(=O)N1CCc2c(C(=O)N3CCC(c4cc(F)ccc4Cl)CC3)n[nH]c2C1 ; CC(=O)N1CCc2c(C(=O)N3CCC(c4cc(F)ccc4C(F)(F)F)CC3)n[nH]c2C1 ; CC(=O)N1CCc2c(C(=O)N3CCC(c4cccc(F)c4C(F)(F)F)CC3)n[nH]c2C1 ; CC(=O)N1CCc2c(C(=O)N3CCC(c4cccc(F)c4Cl)CC3)n[nH]c2C1]

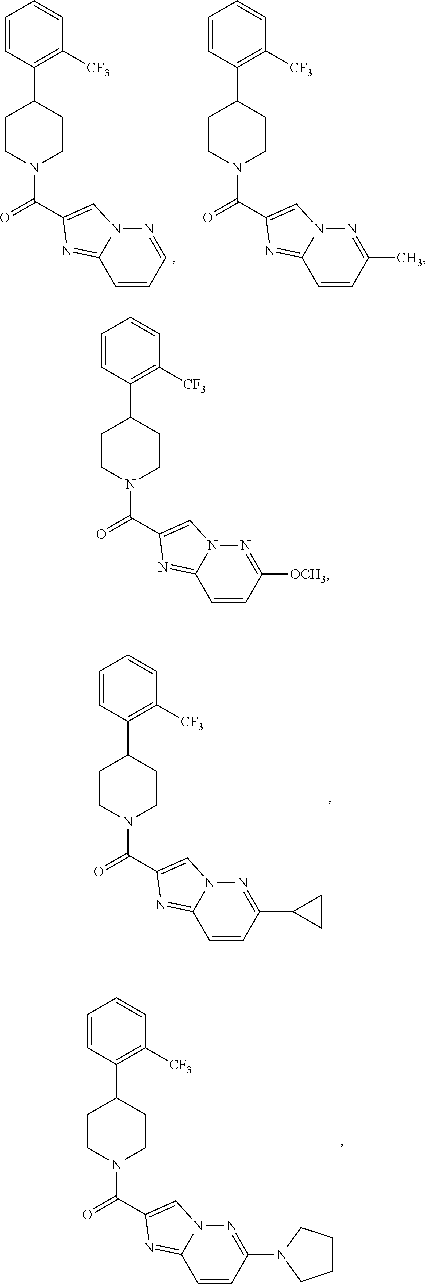

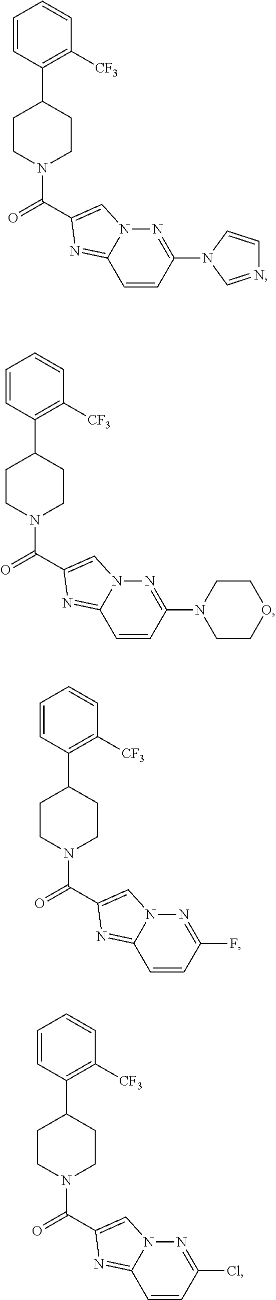 [SMILES: O=C(c1cn2nc(-n3ccnc3)ccc2n1)N1CCC(c2ccccc2C(F)(F)F)CC1 ; O=C(c1cn2nc(N3CCOCC3)ccc2n1)N1CCC(c2ccccc2C(F)(F)F)CC1 ; O=C(c1cn2nc(F)ccc2n1)N1CCC(c2ccccc2C(F)(F)F)CC1 ; O=C(c1cn2nc(Cl)ccc2n1)N1CCC(c2ccccc2C(F)(F)F)CC1]

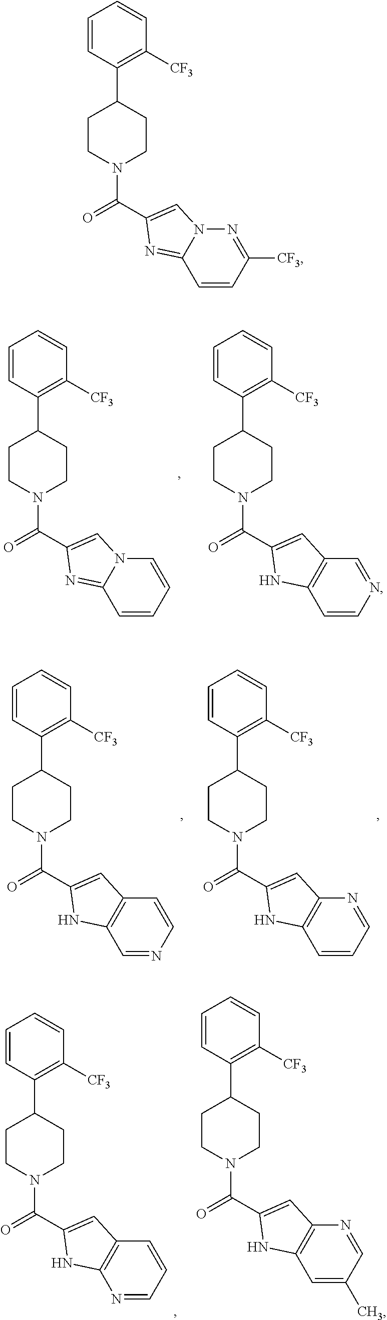

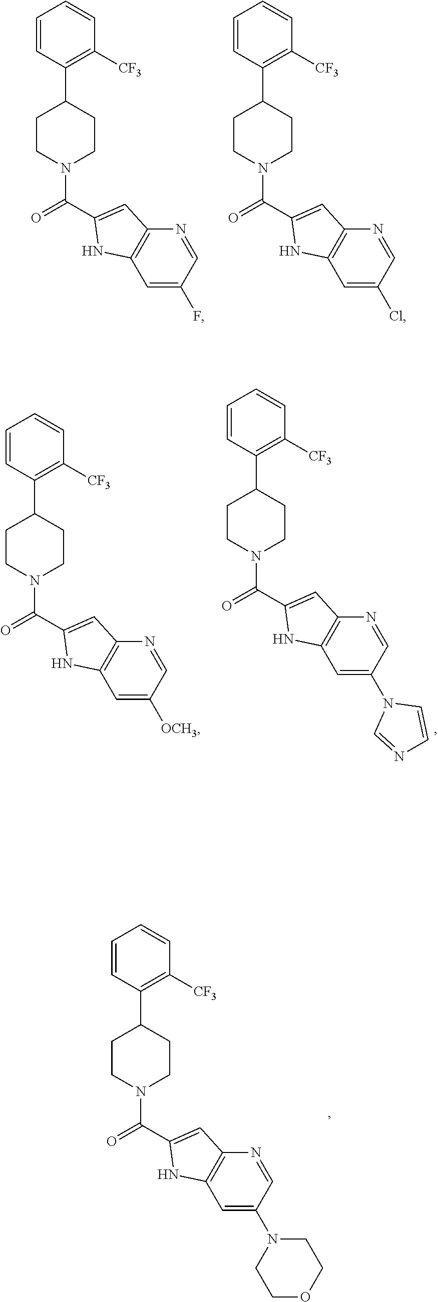 [SMILES: O=C(c1cc2ncc(F)cc2[nH]1)N1CCC(c2ccccc2C(F)(F)F)CC1 ; O=C(c1cc2ncc(Cl)cc2[nH]1)N1CCC(c2ccccc2C(F)(F)F)CC1 ; COc1cnc2cc(C(=O)N3CCC(c4ccccc4C(F)(F)F)CC3)[nH]c2c1 ; O=C(c1cc2ncc(-n3ccnc3)cc2[nH]1)N1CCC(c2ccccc2C(F)(F)F)CC1 ; O=C(c1cc2ncc(N3CCOCC3)cc2[nH]1)N1CCC(c2ccccc2C(F)(F)F)CC1]

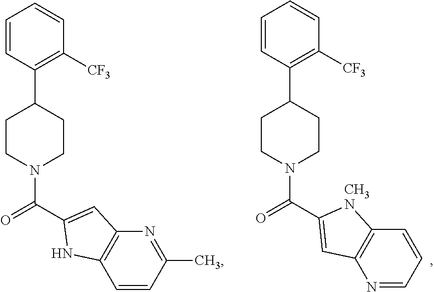

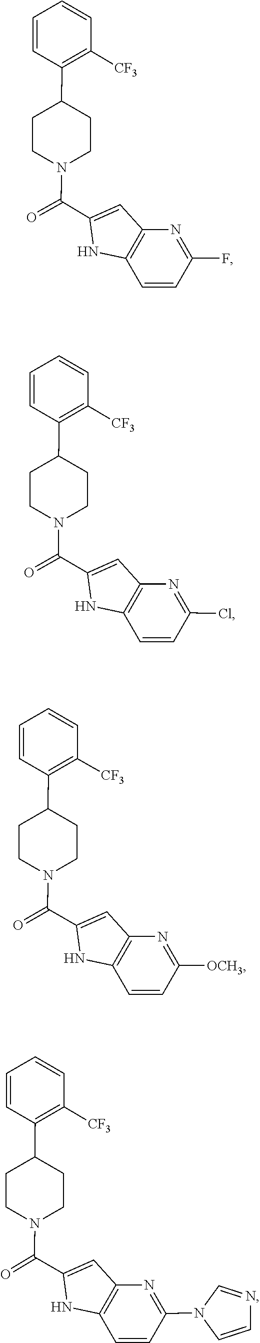 [SMILES: O=C(c1cc2nc(F)ccc2[nH]1)N1CCC(c2ccccc2C(F)(F)F)CC1 ; O=C(c1cc2nc(Cl)ccc2[nH]1)N1CCC(c2ccccc2C(F)(F)F)CC1 ; COc1ccc2[nH]c(C(=O)N3CCC(c4ccccc4C(F)(F)F)CC3)cc2n1 ; O=C(c1cc2nc(-n3ccnc3)ccc2[nH]1)N1CCC(c2ccccc2C(F)(F)F)CC1]

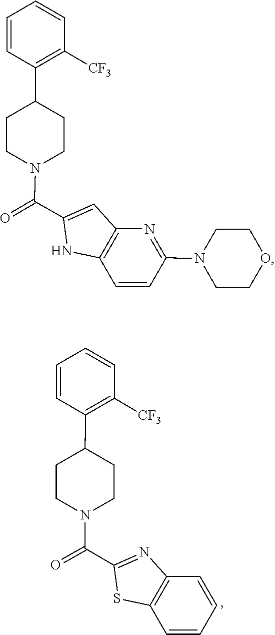 [SMILES: O=C(c1cc2nc(N3CCOCC3)ccc2[nH]1)N1CCC(c2ccccc2C(F)(F)F)CC1 ; O=C(c1nc2ccccc2s1)N1CCC(c2ccccc2C(F)(F)F)CC1]

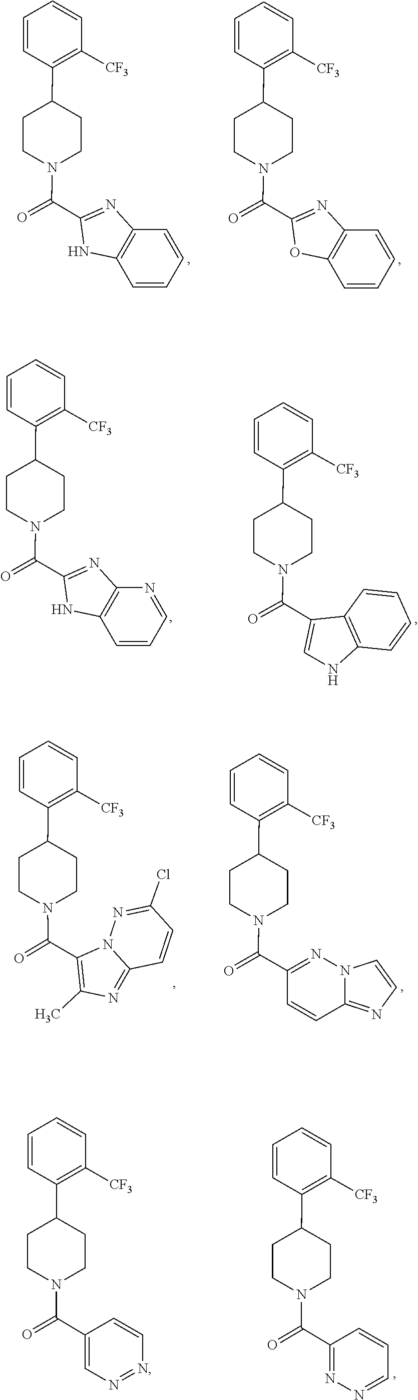

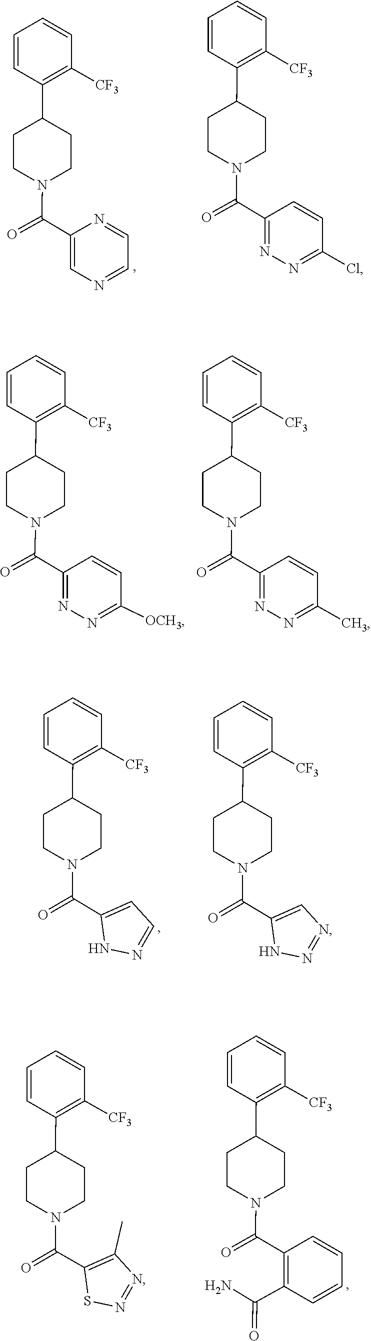

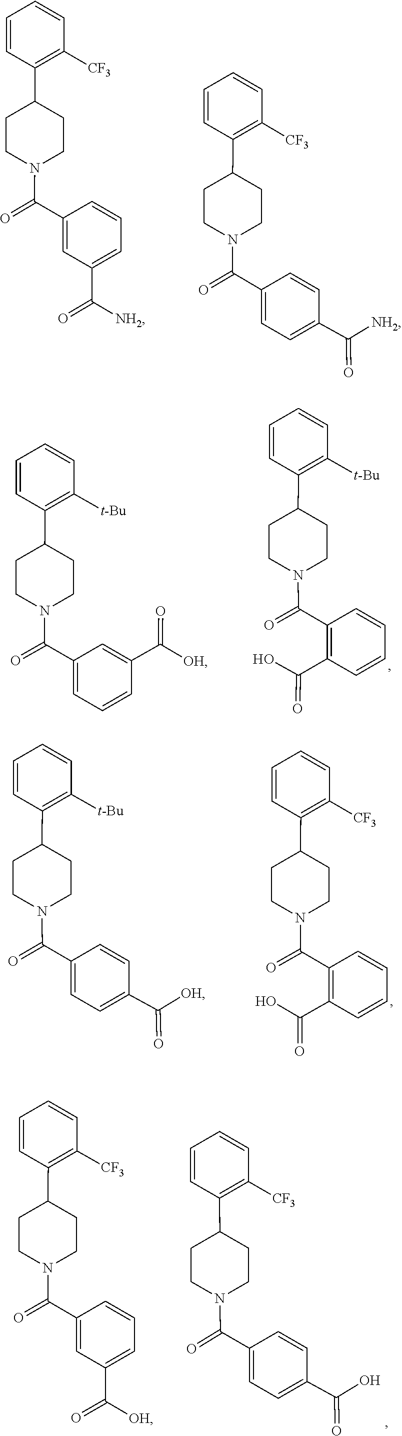

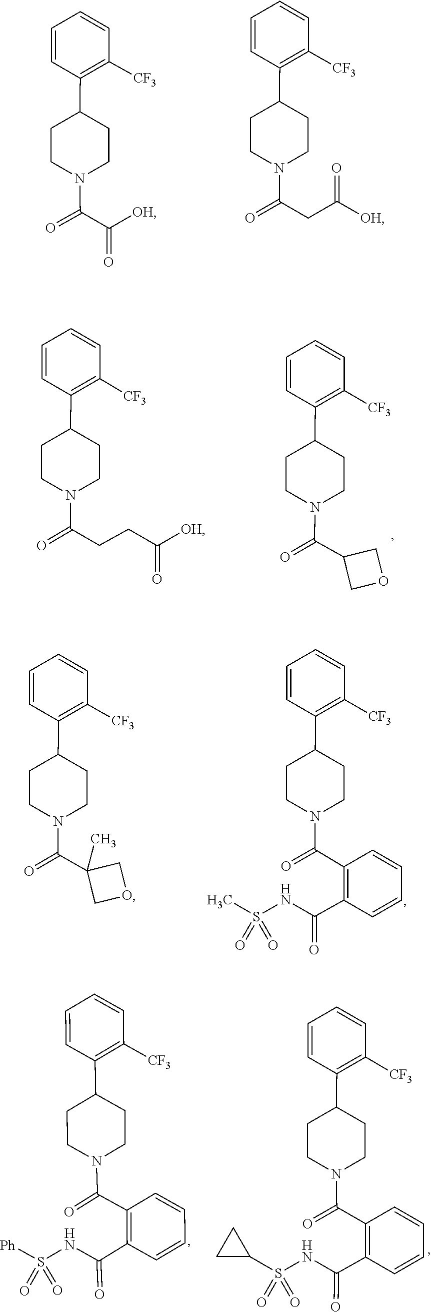

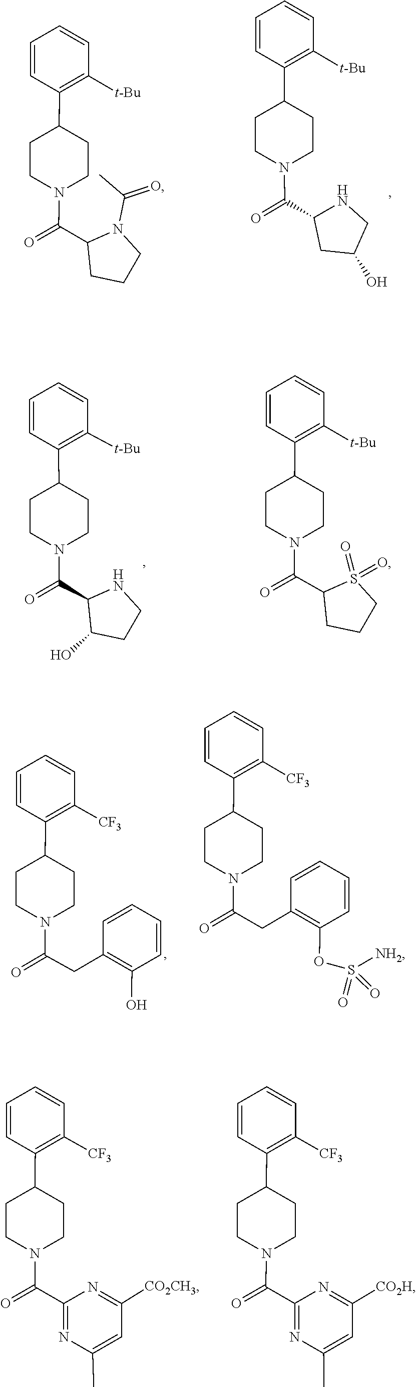

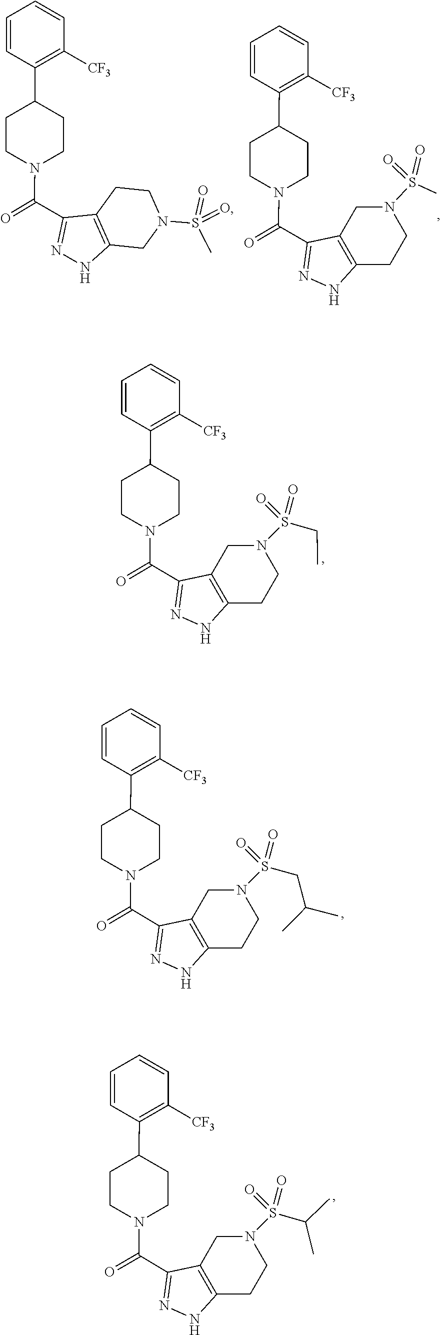

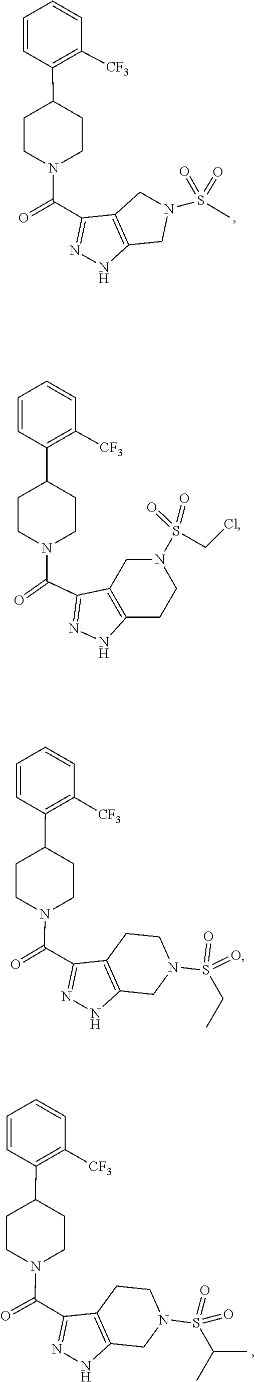 [SMILES: CS(=O)(=O)N1Cc2[nH]nc(C(=O)N3CCC(c4ccccc4C(F)(F)F)CC3)c2C1 ; O=C(c1n[nH]c2c1CN(S(=O)(=O)CCl)CC2)N1CCC(c2ccccc2C(F)(F)F)CC1 ; CCS(=O)(=O)N1CCc2c(C(=O)N3CCC(c4ccccc4C(F)(F)F)CC3)n[nH]c2C1 ; CC(C)S(=O)(=O)N1CCc2c(C(=O)N3CCC(c4ccccc4C(F)(F)F)CC3)n[nH]c2C1]

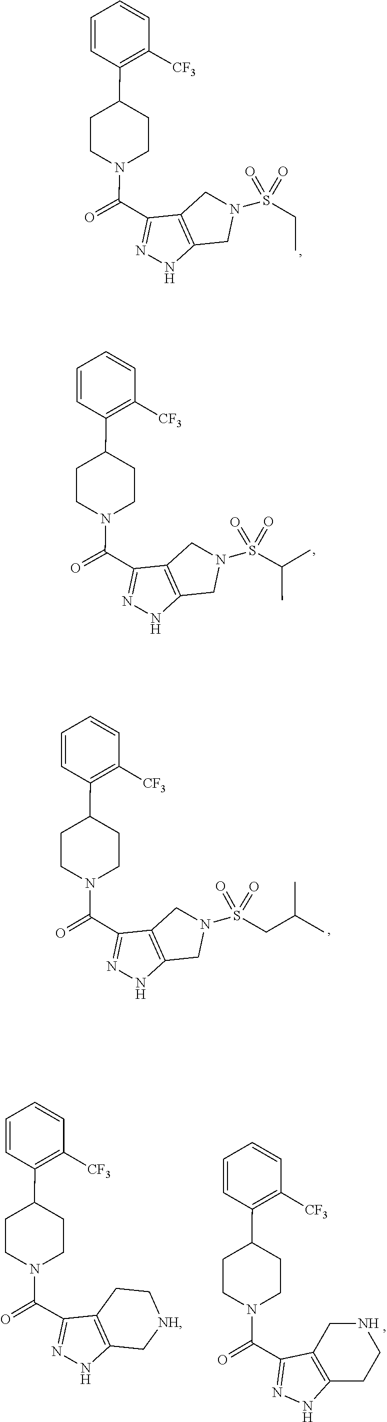 [SMILES: CCS(=O)(=O)N1Cc2[nH]nc(C(=O)N3CCC(c4ccccc4C(F)(F)F)CC3)c2C1 ; CC(C)S(=O)(=O)N1Cc2[nH]nc(C(=O)N3CCC(c4ccccc4C(F)(F)F)CC3)c2C1 ; CC(C)CS(=O)(=O)N1Cc2[nH]nc(C(=O)N3CCC(c4ccccc4C(F)(F)F)CC3)c2C1 ; O=C(c1n[nH]c2c1CCNC2)N1CCC(c2ccccc2C(F)(F)F)CC1 ; O=C(c1n[nH]c2c1CNCC2)N1CCC(c2ccccc2C(F)(F)F)CC1]

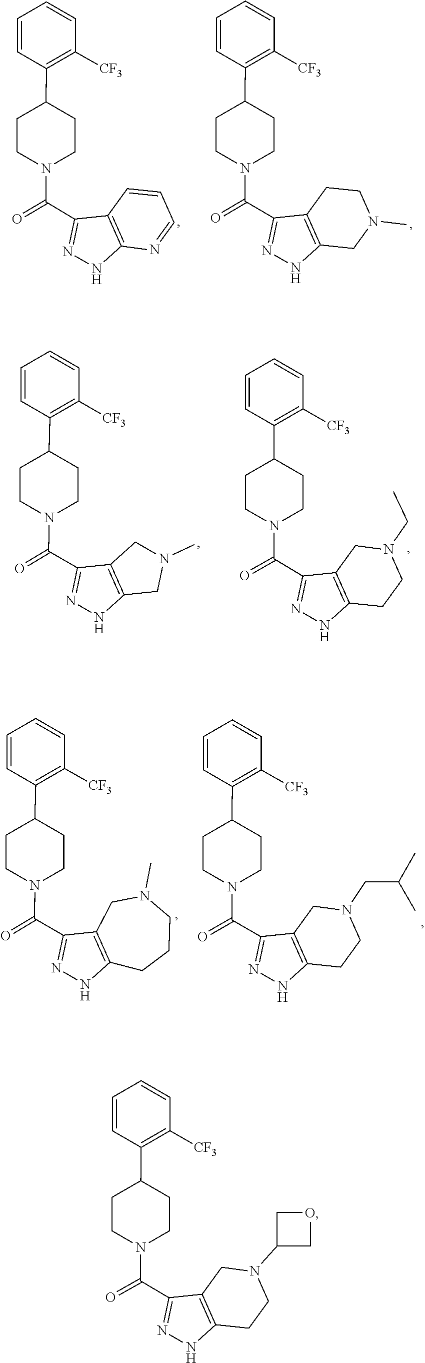

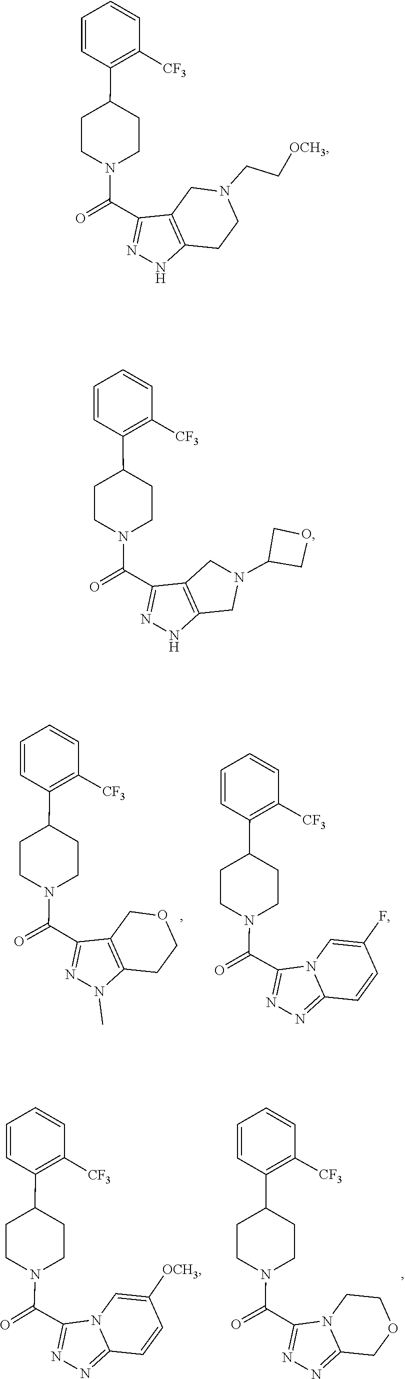 [SMILES: COCCN1CCc2[nH]nc(C(=O)N3CCC(c4ccccc4C(F)(F)F)CC3)c2C1 ; O=C(c1n[nH]c2c1CN(C1COC1)C2)N1CCC(c2ccccc2C(F)(F)F)CC1 ; Cn1nc(C(=O)N2CCC(c3ccccc3C(F)(F)F)CC2)c2c1CCOC2 ; O=C(c1nnc2ccc(F)cn12)N1CCC(c2ccccc2C(F)(F)F)CC1 ; COc1ccc2nnc(C(=O)N3CCC(c4ccccc4C(F)(F)F)CC3)n2c1 ; O=C(c1nnc2n1CCOC2)N1CCC(c2ccccc2C(F)(F)F)CC1]

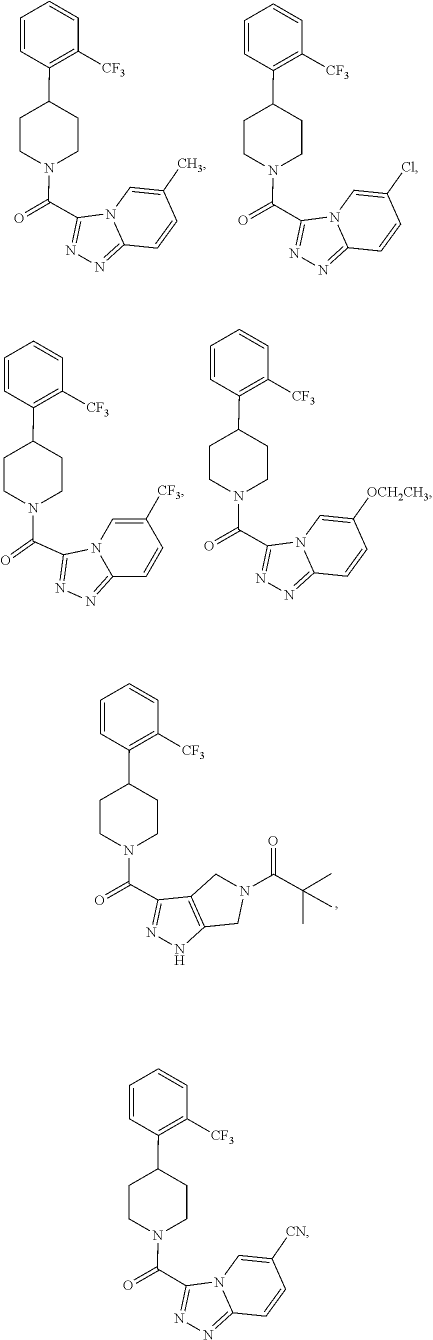 [SMILES: Cc1ccc2nnc(C(=O)N3CCC(c4ccccc4C(F)(F)F)CC3)n2c1 ; O=C(c1nnc2ccc(Cl)cn12)N1CCC(c2ccccc2C(F)(F)F)CC1 ; O=C(c1nnc2ccc(C(F)(F)F)cn12)N1CCC(c2ccccc2C(F)(F)F)CC1 ; CCOc1ccc2nnc(C(=O)N3CCC(c4ccccc4C(F)(F)F)CC3)n2c1 ; CC(C)(C)C(=O)N1Cc2[nH]nc(C(=O)N3CCC(c4ccccc4C(F)(F)F)CC3)c2C1 ; N#Cc1ccc2nnc(C(=O)N3CCC(c4ccccc4C(F)(F)F)CC3)n2c1]

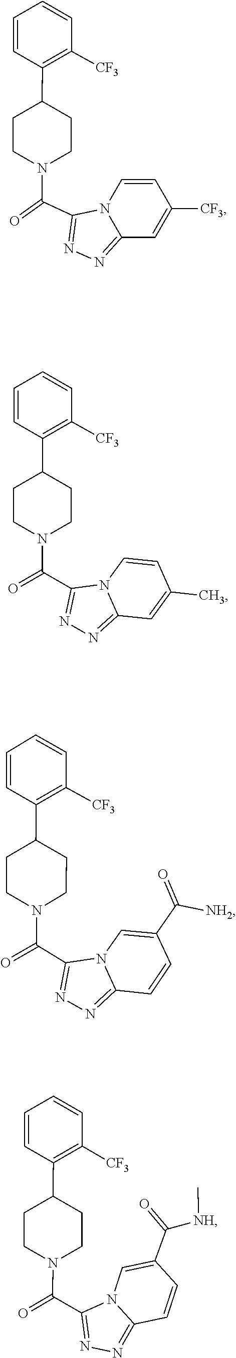 [SMILES: O=C(c1nnc2cc(C(F)(F)F)ccn12)N1CCC(c2ccccc2C(F)(F)F)CC1 ; Cc1ccn2c(C(=O)N3CCC(c4ccccc4C(F)(F)F)CC3)nnc2c1 ; NC(=O)c1ccc2nnc(C(=O)N3CCC(c4ccccc4C(F)(F)F)CC3)n2c1 ; CNC(=O)c1ccc2nnc(C(=O)N3CCC(c4ccccc4C(F)(F)F)CC3)n2c1]

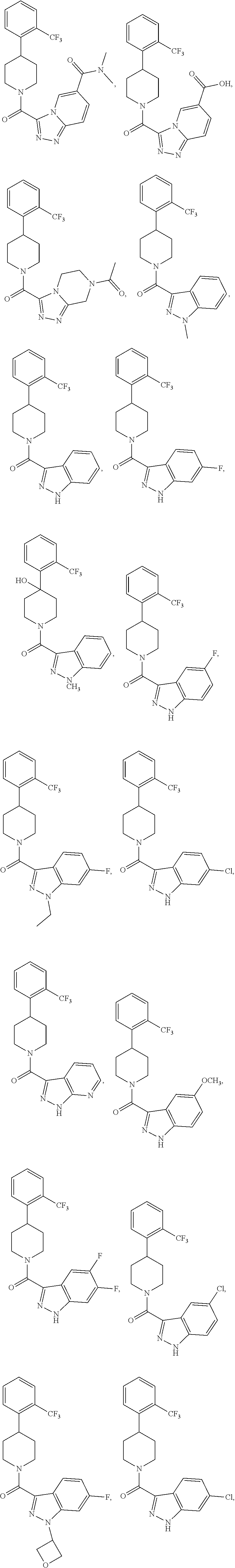

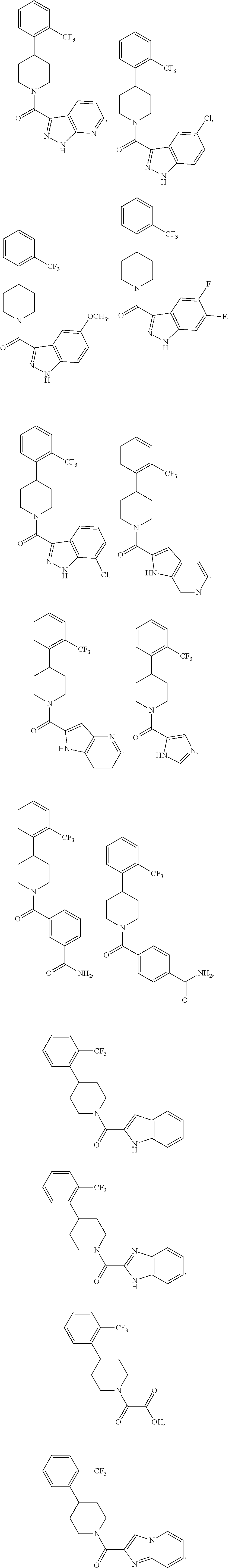

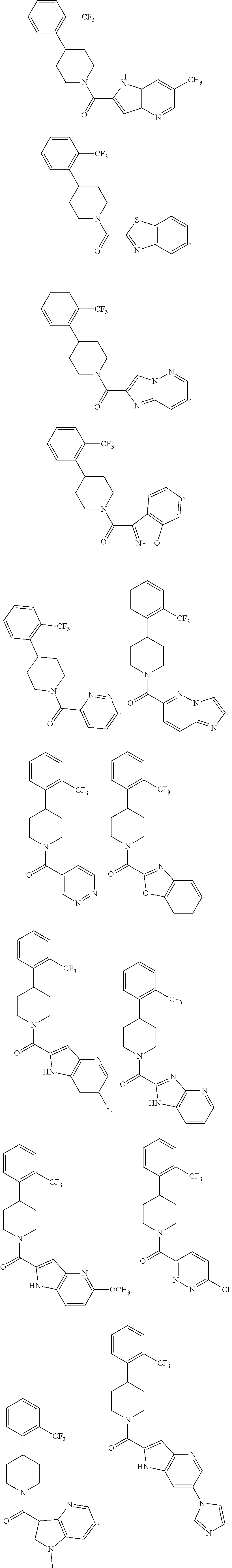

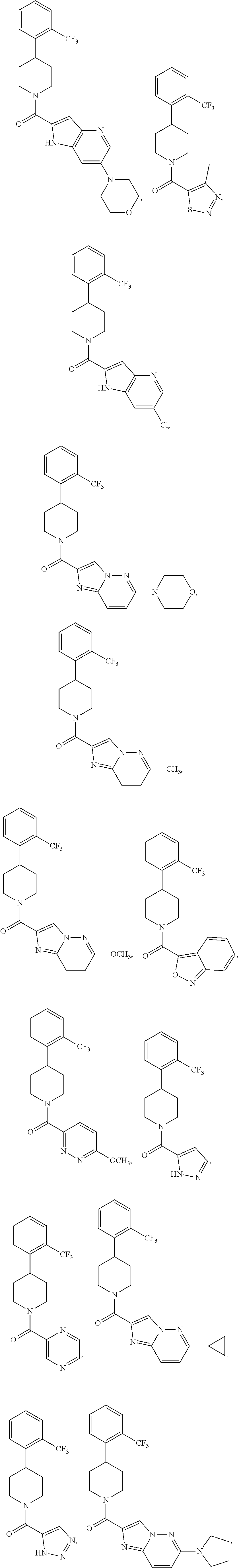

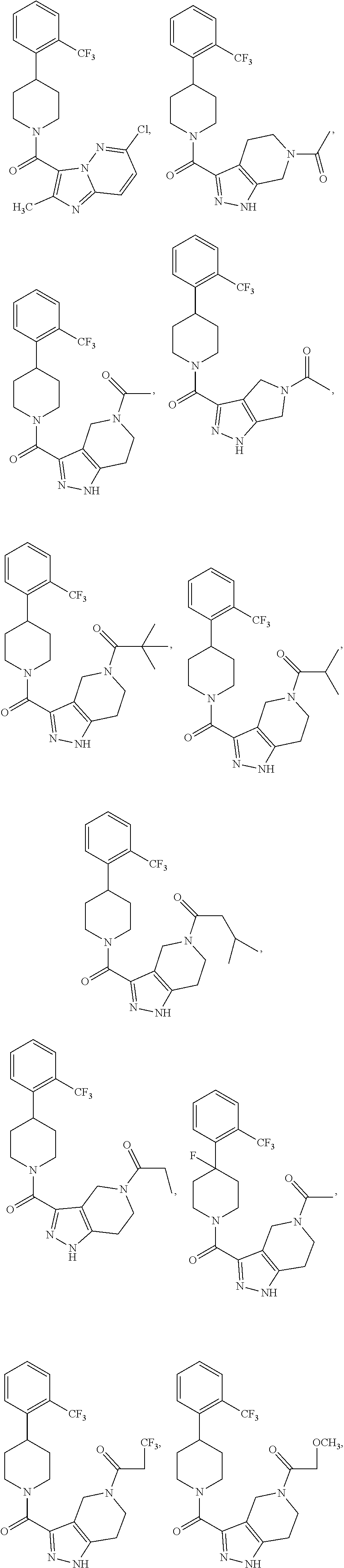

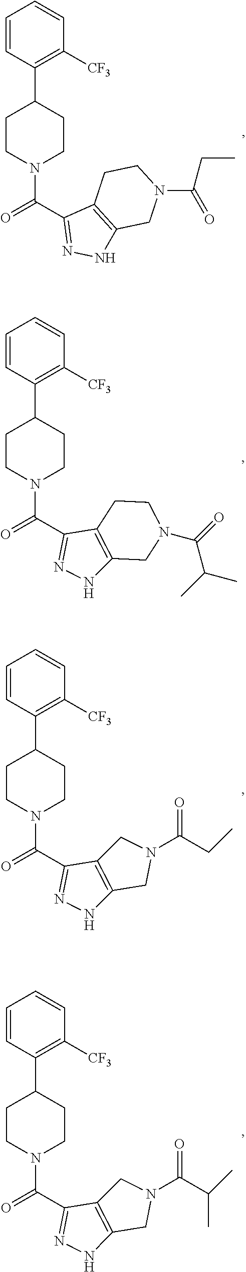 [SMILES: CCC(=O)N1CCc2c(C(=O)N3CCC(c4ccccc4C(F)(F)F)CC3)n[nH]c2C1 ; CC(C)C(=O)N1CCc2c(C(=O)N3CCC(c4ccccc4C(F)(F)F)CC3)n[nH]c2C1 ; CCC(=O)N1Cc2[nH]nc(C(=O)N3CCC(c4ccccc4C(F)(F)F)CC3)c2C1 ; CC(C)C(=O)N1Cc2[nH]nc(C(=O)N3CCC(c4ccccc4C(F)(F)F)CC3)c2C1]

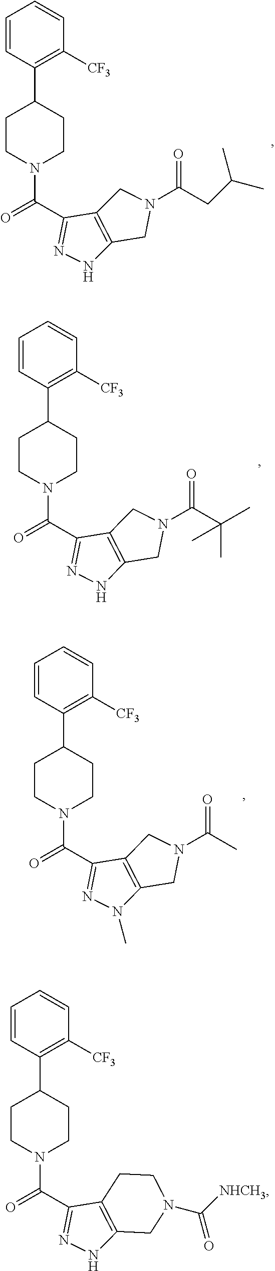 [SMILES: CC(C)CC(=O)N1Cc2[nH]nc(C(=O)N3CCC(c4ccccc4C(F)(F)F)CC3)c2C1 ; CC(C)(C)C(=O)N1Cc2[nH]nc(C(=O)N3CCC(c4ccccc4C(F)(F)F)CC3)c2C1 ; CC(=O)N1Cc2c(C(=O)N3CCC(c4ccccc4C(F)(F)F)CC3)nn(C)c2C1 ; CNC(=O)N1CCc2c(C(=O)N3CCC(c4ccccc4C(F)(F)F)CC3)n[nH]c2C1]

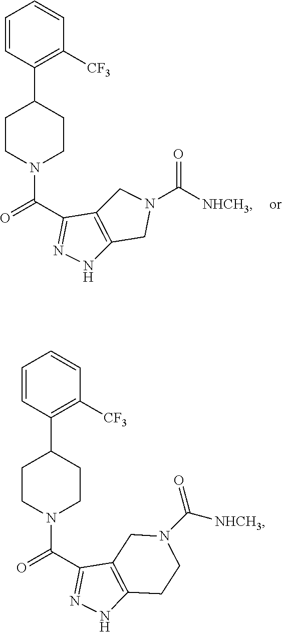 [SMILES: CNC(=O)N1Cc2[nH]nc(C(=O)N3CCC(c4ccccc4C(F)(F)F)CC3)c2C1 ; CNC(=O)N1CCc2[nH]nc(C(=O)N3CCC(c4ccccc4C(F)(F)F)CC3)c2C1]

or a pharmaceutically acceptable salt thereof.

In some embodiments, the selective RBP4 antagonist is

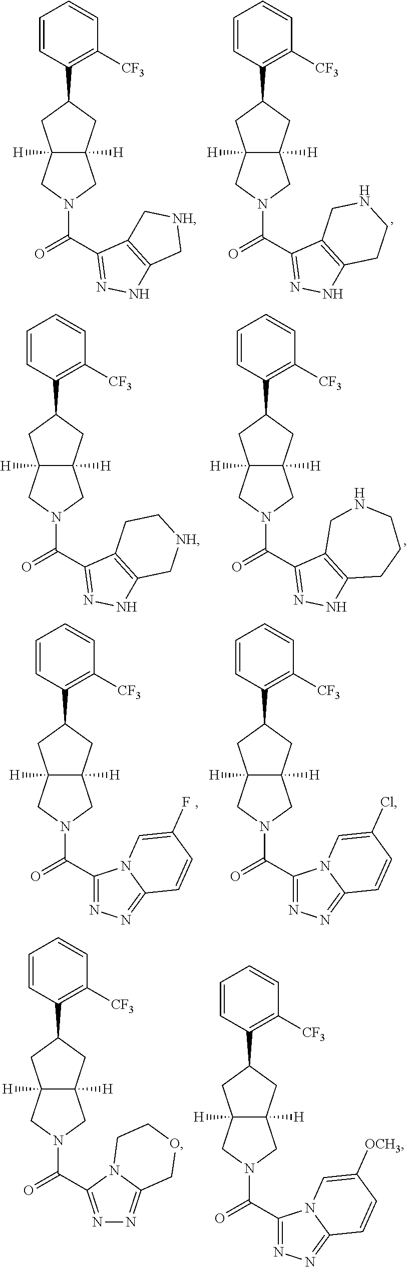

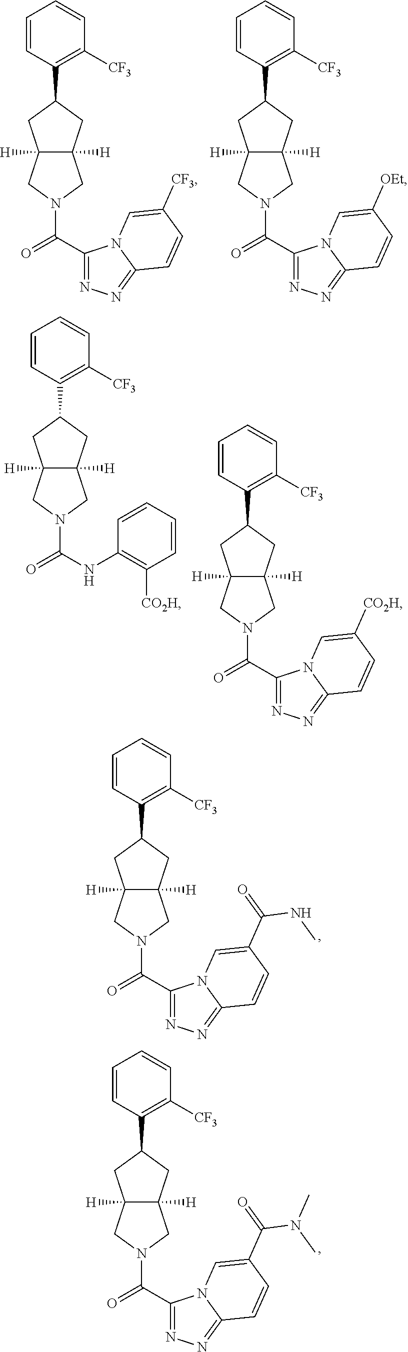

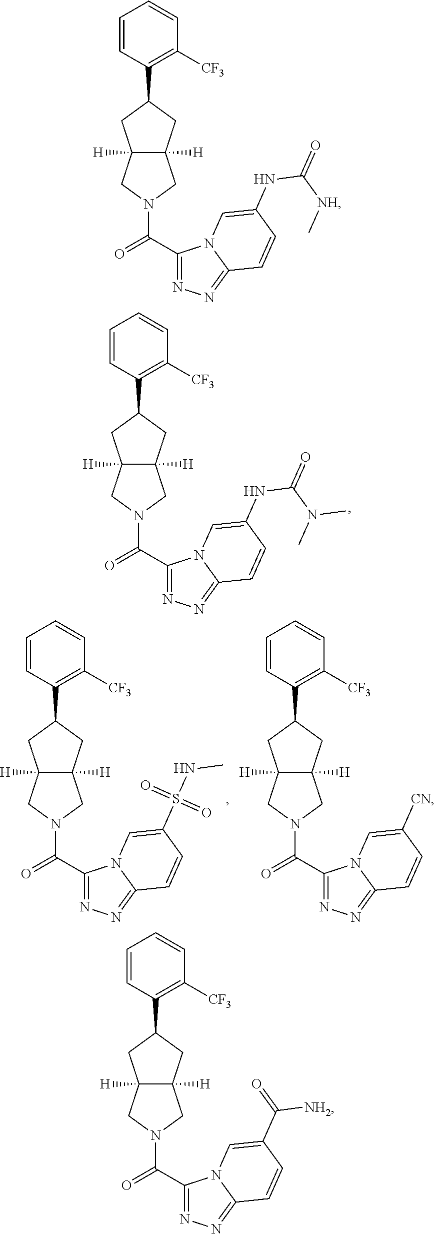

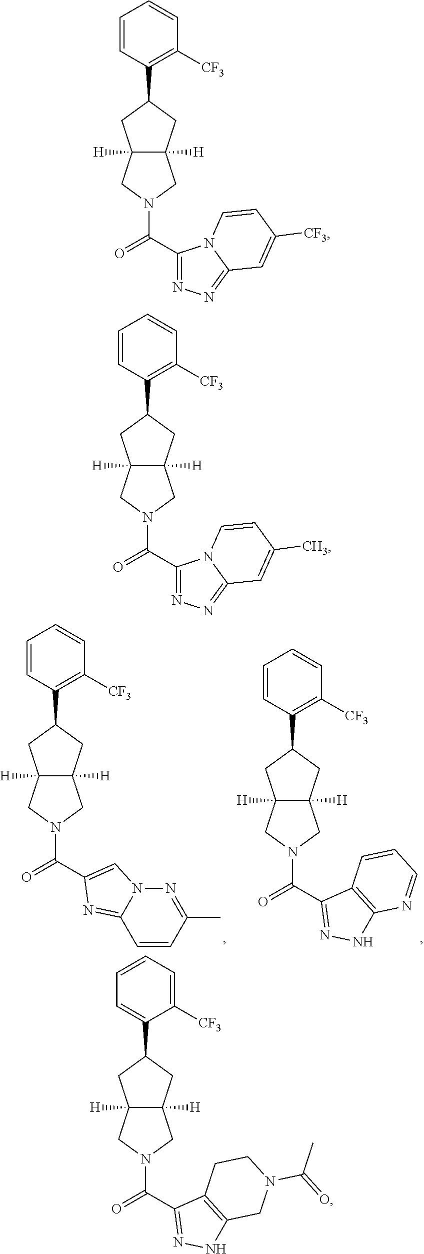

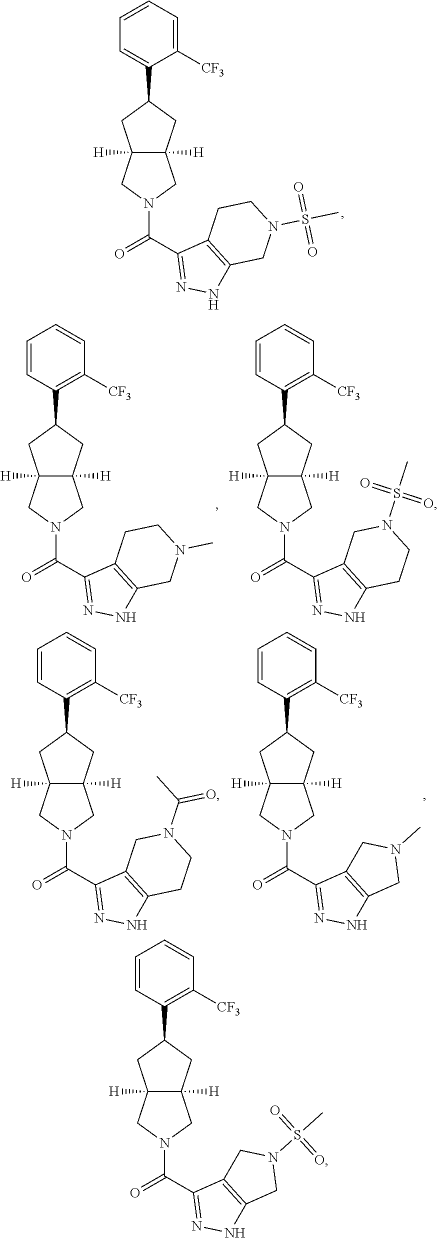

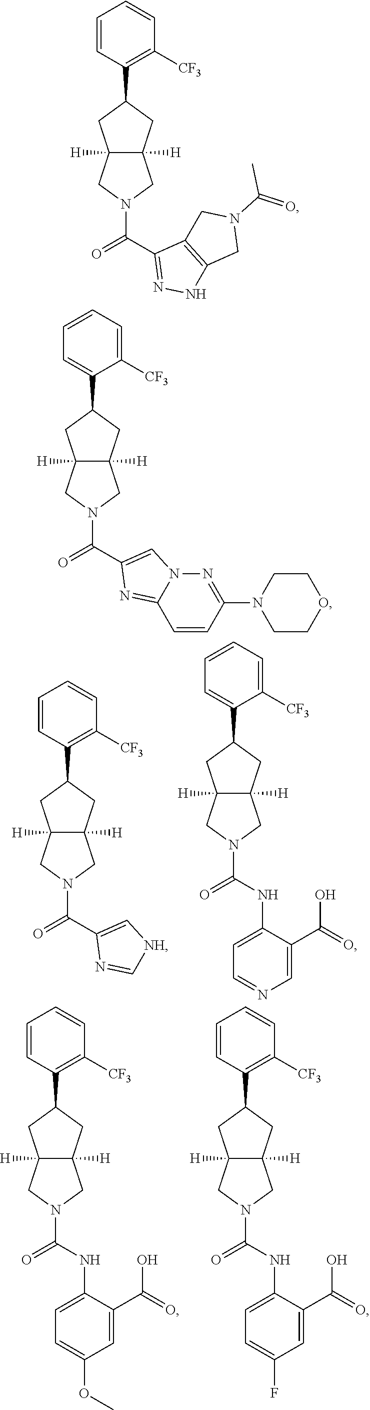

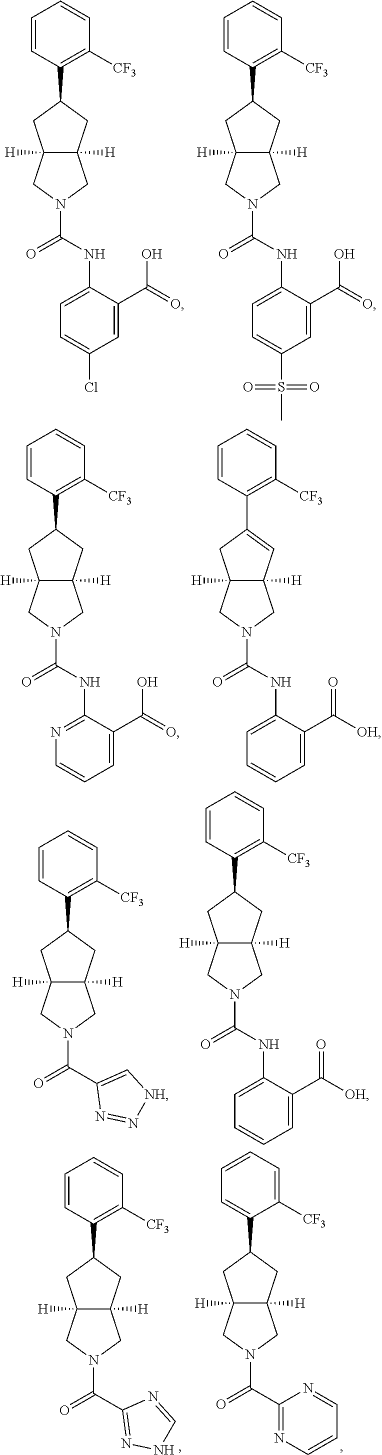

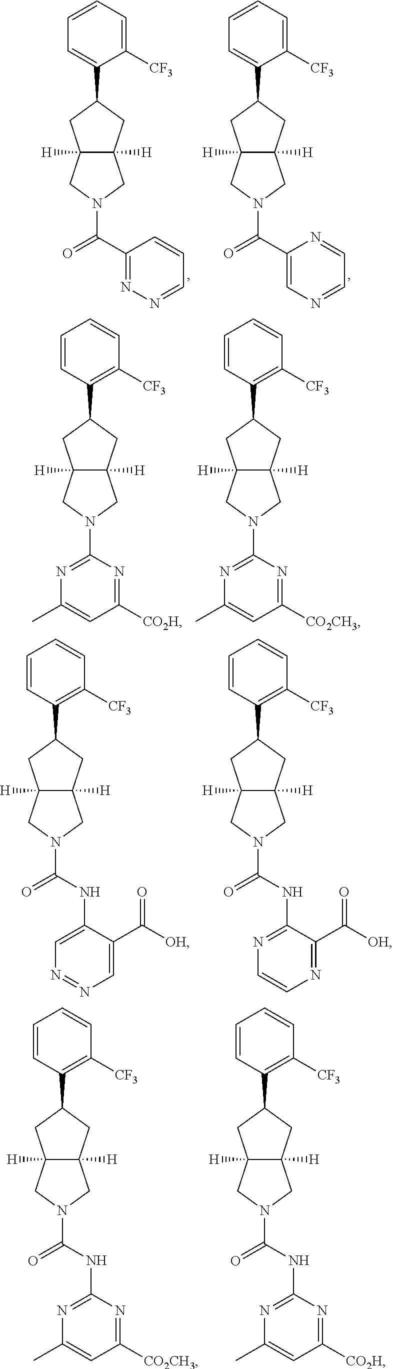

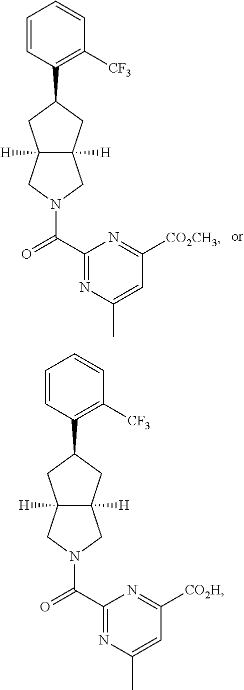

or a pharmaceutically acceptable salt thereof.

In some embodiments, the selective RBP4 antagonist is

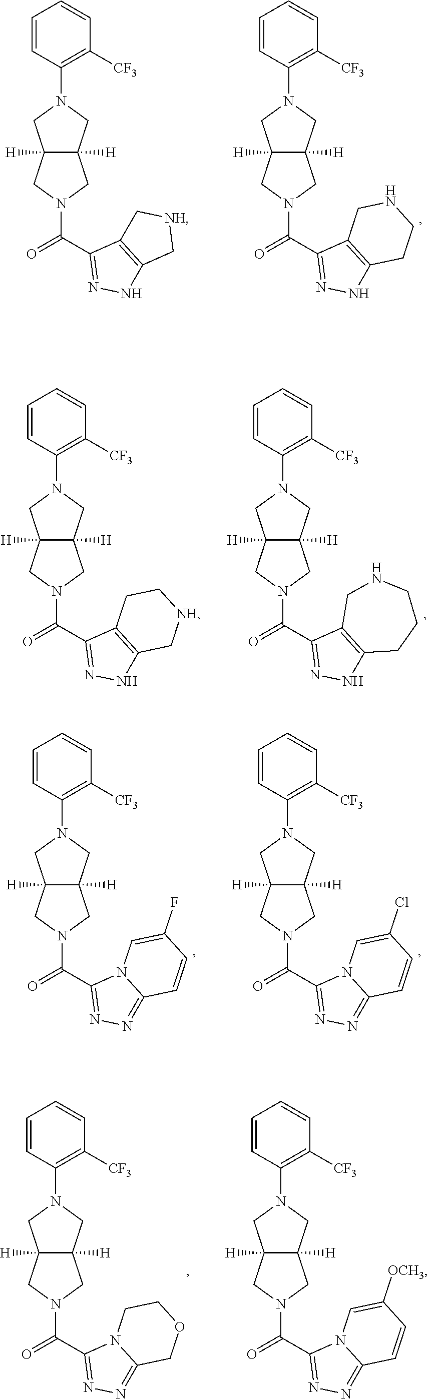

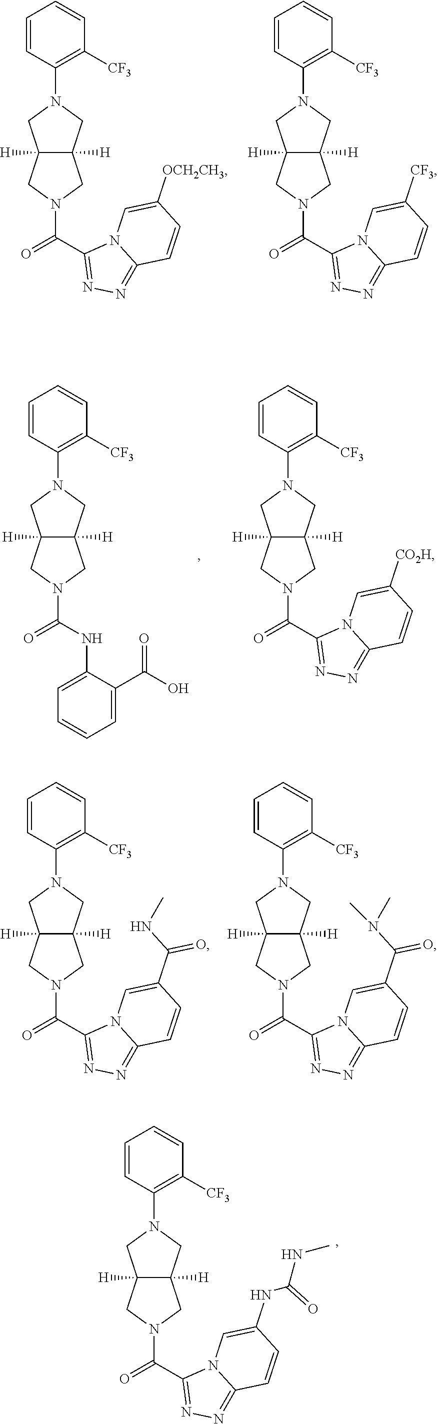

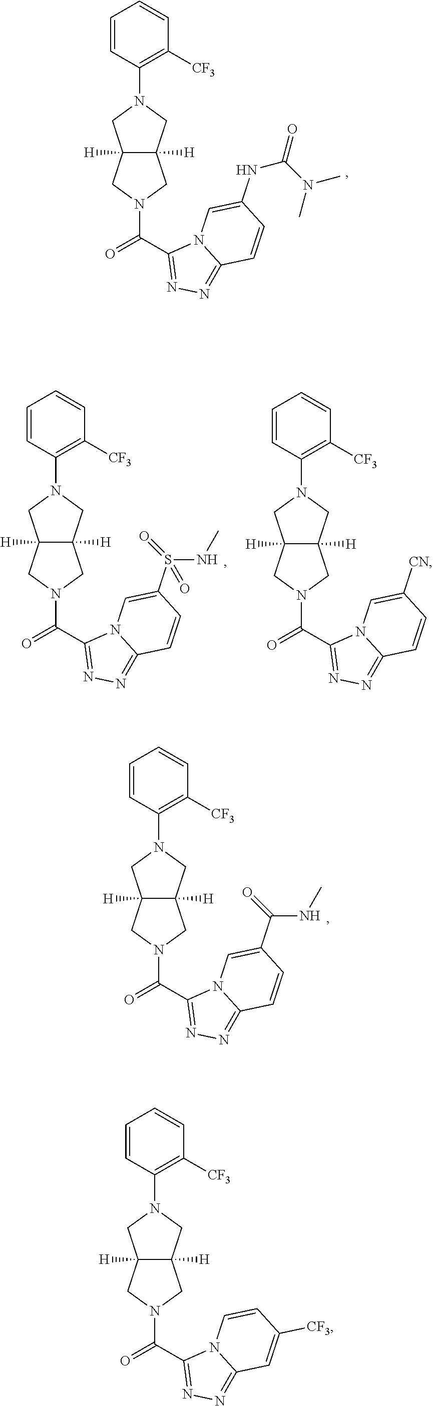

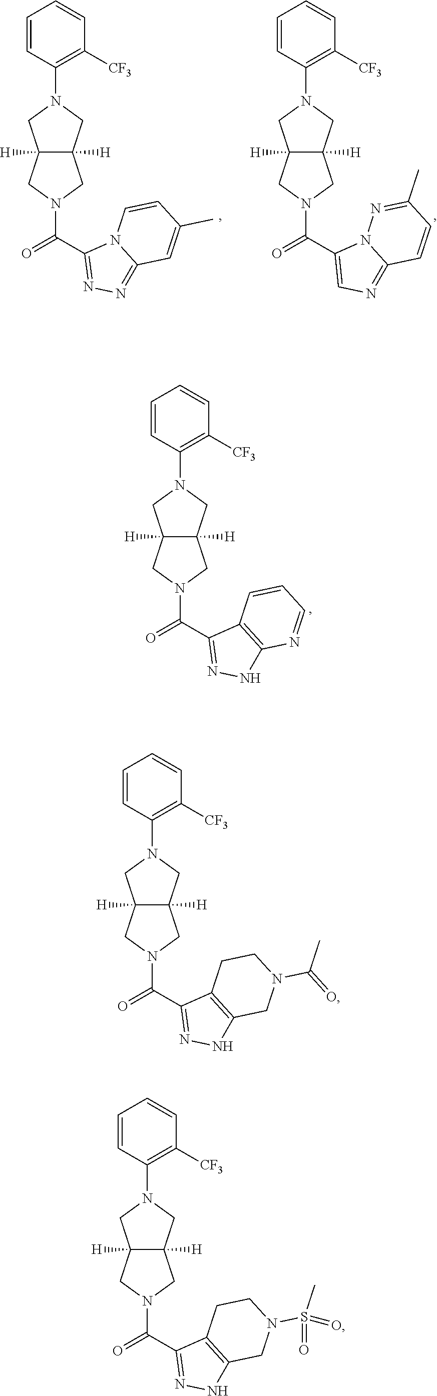 [SMILES: Cc1ccn2c(C(=O)N3C[C@@H]4CN(c5ccccc5C(F)(F)F)C[C@@H]4C3)nnc2c1 ; Cc1ccc2ncc(C(=O)N3C[C@@H]4CN(c5ccccc5C(F)(F)F)C[C@@H]4C3)n2n1 ; O=C(c1n[nH]c2ncccc12)N1C[C@@H]2CN(c3ccccc3C(F)(F)F)C[C@@H]2C1 ; CC(=O)N1CCc2c(C(=O)N3C[C@@H]4CN(c5ccccc5C(F)(F)F)C[C@@H]4C3)n[nH]c2C1 ; CS(=O)(=O)N1CCc2c(C(=O)N3C[C@@H]4CN(c5ccccc5C(F)(F)F)C[C@@H]4C3)n[nH]c2C1]

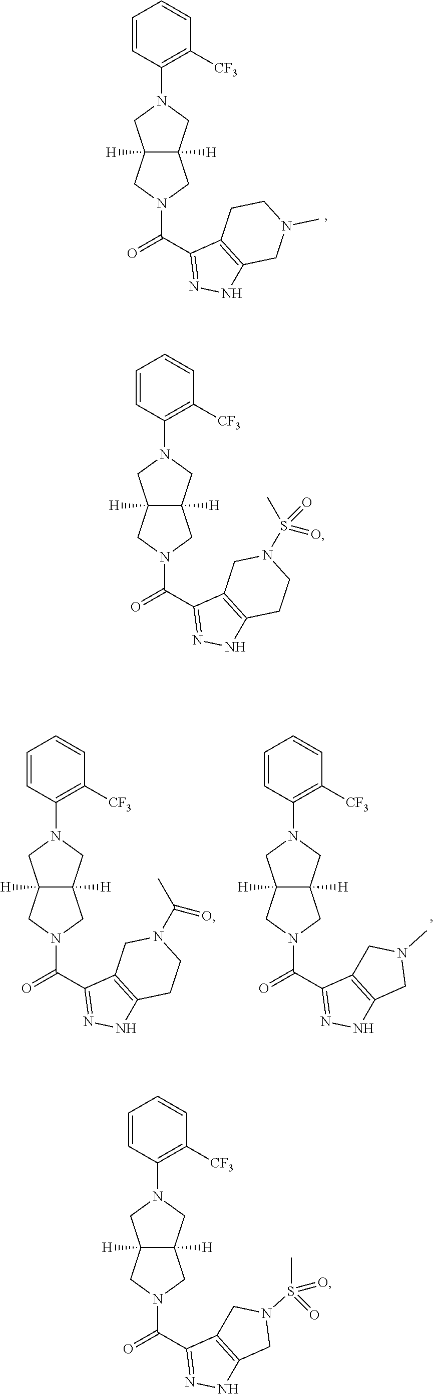

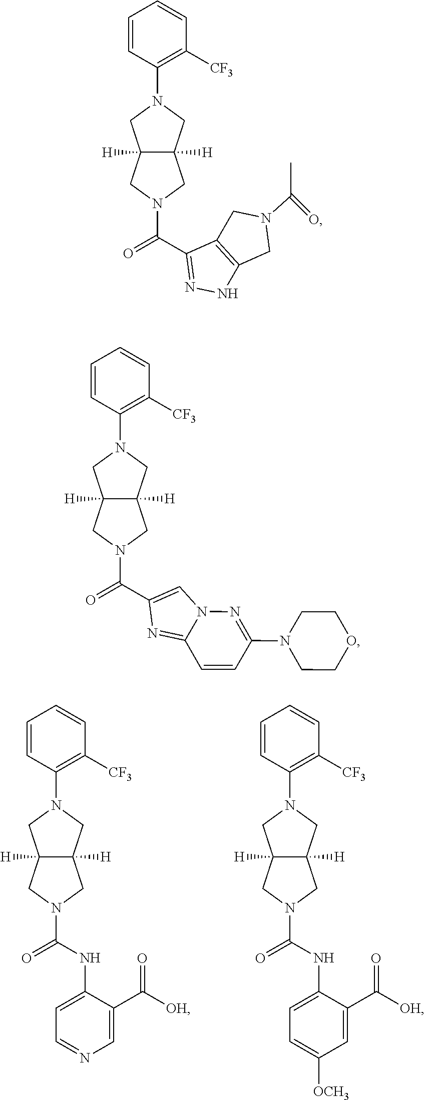

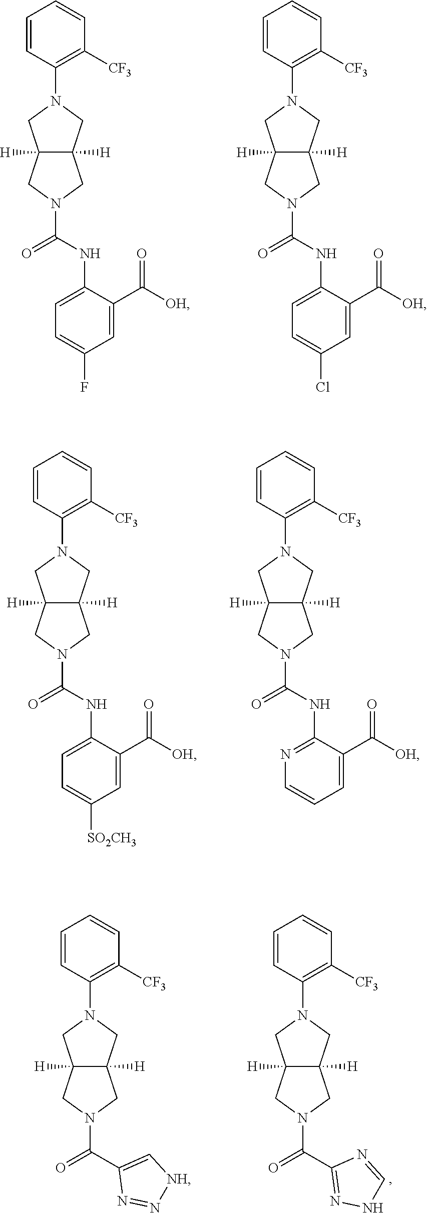 [SMILES: O=C(O)c1cc(F)ccc1NC(=O)N1C[C@@H]2CN(c3ccccc3C(F)(F)F)C[C@@H]2C1 ; O=C(O)c1cc(Cl)ccc1NC(=O)N1C[C@@H]2CN(c3ccccc3C(F)(F)F)C[C@@H]2C1 ; CS(=O)(=O)c1ccc(NC(=O)N2C[C@@H]3CN(c4ccccc4C(F)(F)F)C[C@@H]3C2)c(C(=O)O)c1 ; O=C(O)c1cccnc1NC(=O)N1C[C@@H]2CN(c3ccccc3C(F)(F)F)C[C@@H]2C1 ; O=C(c1c[nH]nn1)N1C[C@@H]2CN(c3ccccc3C(F)(F)F)C[C@@H]2C1 ; O=C(c1nc[nH]n1)N1C[C@@H]2CN(c3ccccc3C(F)(F)F)C[C@@H]2C1]

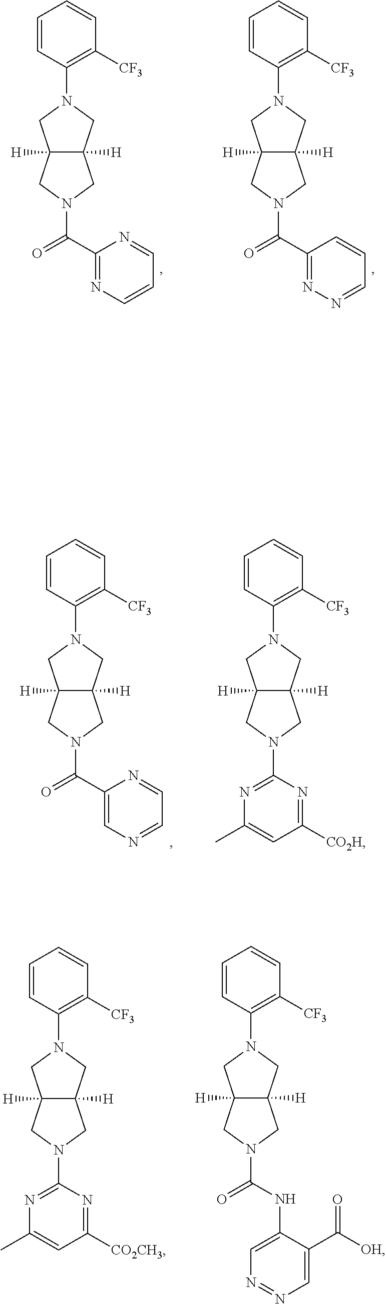 [SMILES: O=C(c1ncccn1)N1C[C@@H]2CN(c3ccccc3C(F)(F)F)C[C@@H]2C1 ; O=C(c1cccnn1)N1C[C@@H]2CN(c3ccccc3C(F)(F)F)C[C@@H]2C1 ; O=C(c1cnccn1)N1C[C@@H]2CN(c3ccccc3C(F)(F)F)C[C@@H]2C1 ; Cc1cc(C(=O)O)nc(N2C[C@@H]3CN(c4ccccc4C(F)(F)F)C[C@@H]3C2)n1 ; COC(=O)c1cc(C)nc(N2C[C@@H]3CN(c4ccccc4C(F)(F)F)C[C@@H]3C2)n1 ; O=C(O)c1cnncc1NC(=O)N1C[C@@H]2CN(c3ccccc3C(F)(F)F)C[C@@H]2C1]

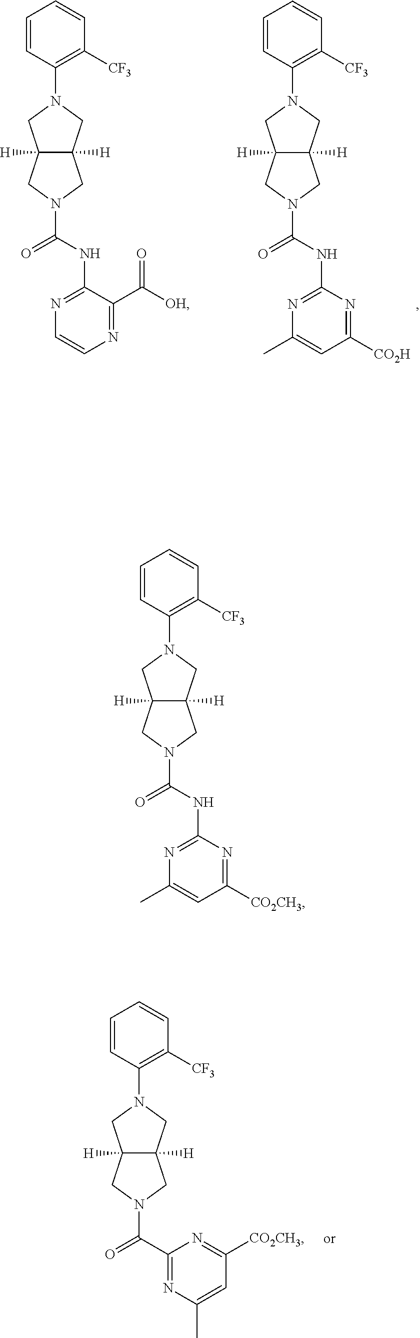 [SMILES: O=C(O)c1nccnc1NC(=O)N1C[C@@H]2CN(c3ccccc3C(F)(F)F)C[C@@H]2C1 ; Cc1cc(C(=O)O)nc(NC(=O)N2C[C@@H]3CN(c4ccccc4C(F)(F)F)C[C@@H]3C2)n1 ; COC(=O)c1cc(C)nc(NC(=O)N2C[C@@H]3CN(c4ccccc4C(F)(F)F)C[C@@H]3C2)n1 ; COC(=O)c1cc(C)nc(C(=O)N2C[C@@H]3CN(c4ccccc4C(F)(F)F)C[C@@H]3C2)n1]

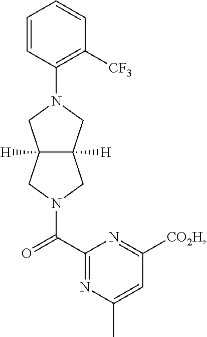 [SMILES: Cc1cc(C(=O)O)nc(C(=O)N2C[C@@H]3CN(c4ccccc4C(F)(F)F)C[C@@H]3C2)n1]

or a pharmaceutically acceptable salt thereof.

In some embodiments, the selective RBP4 antagonist is

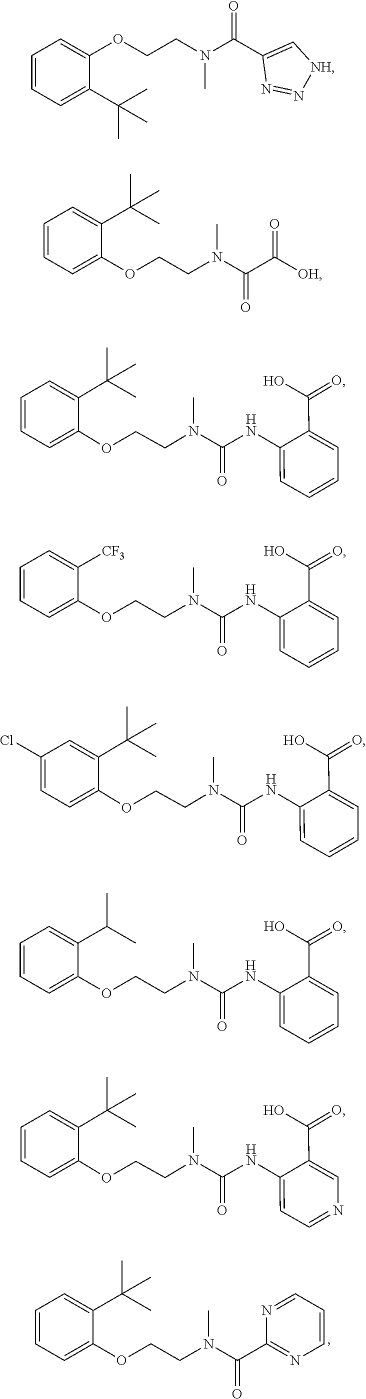

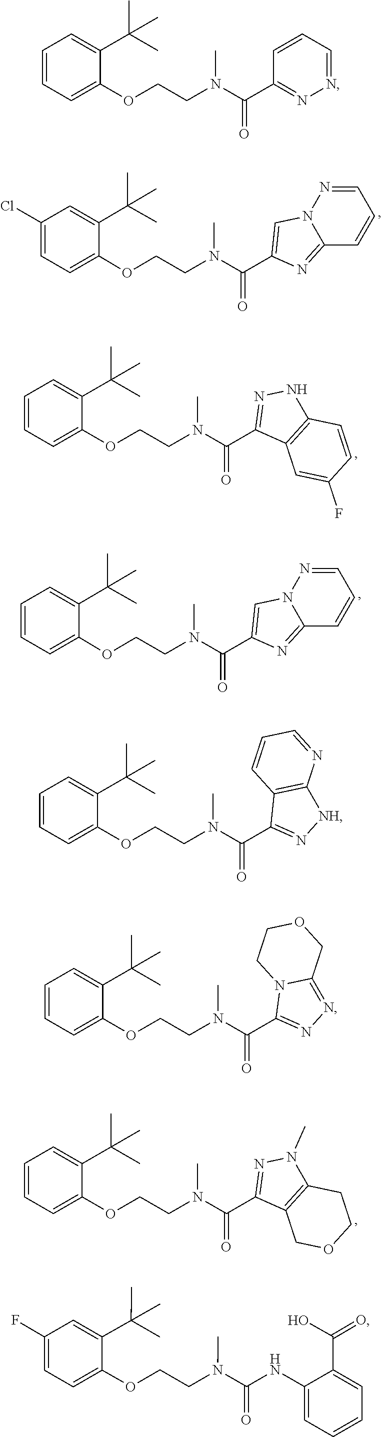

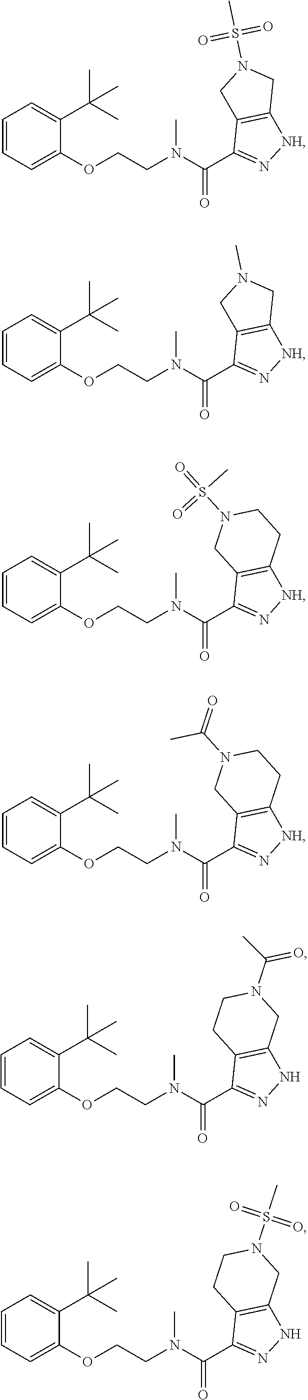

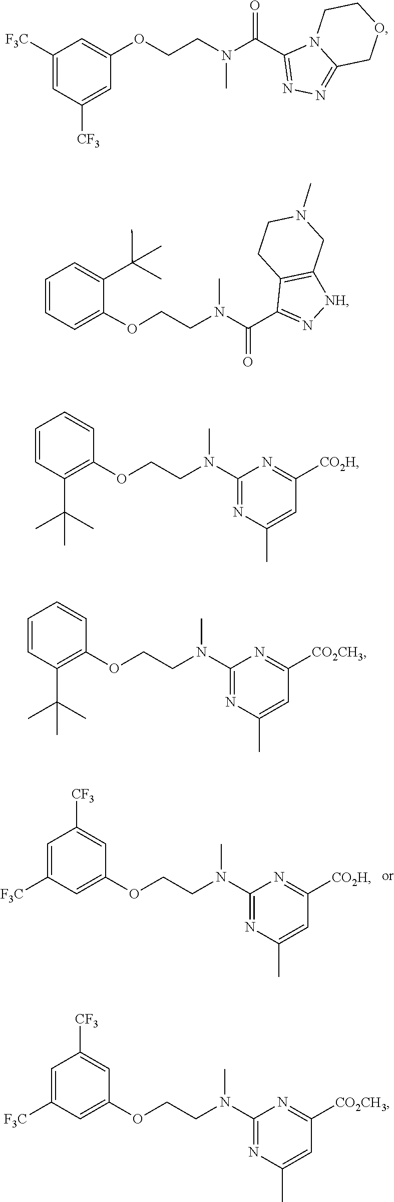

or a pharmaceutically acceptable salt thereof.

In some embodiments, the selective RBP4 antagonist is

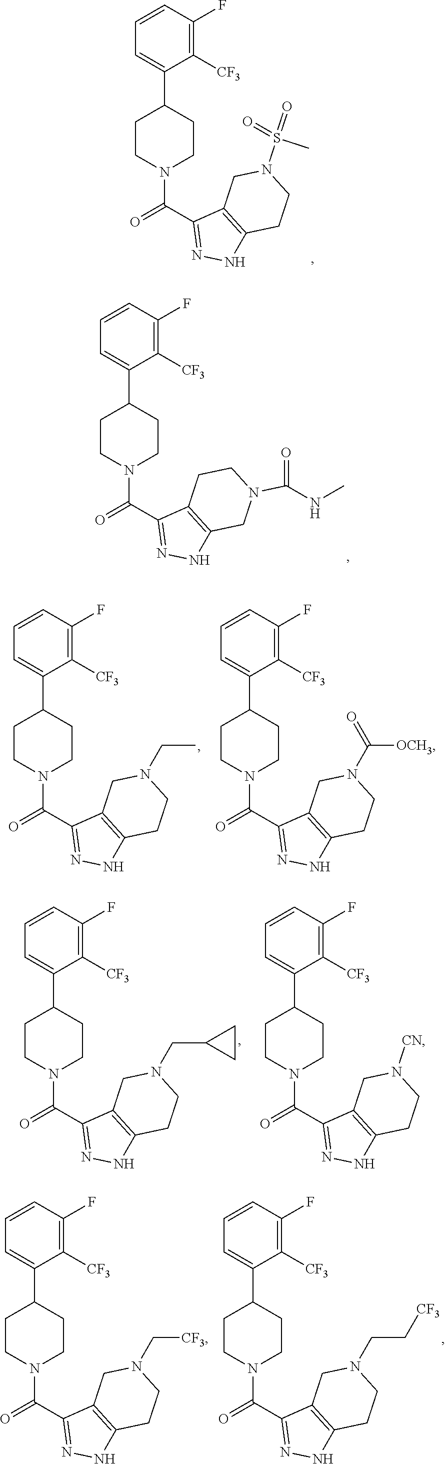

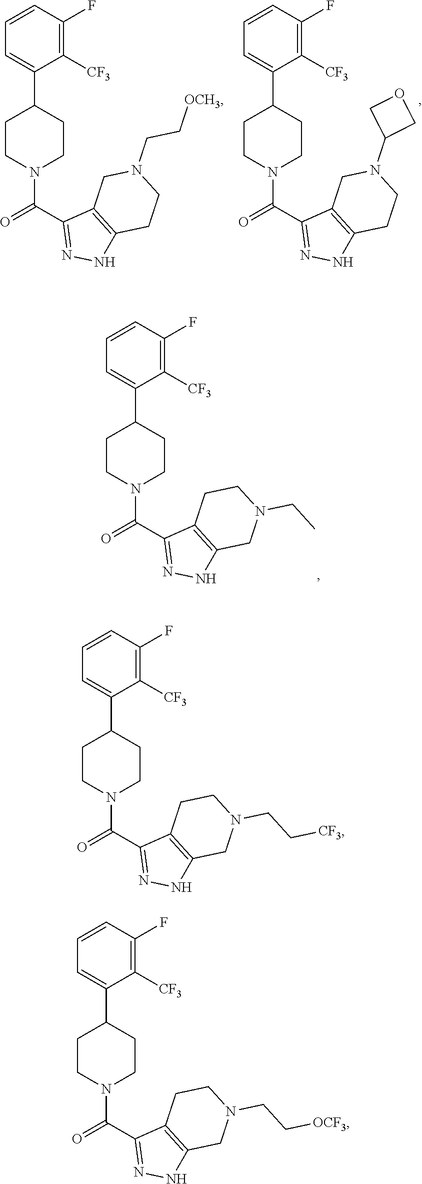

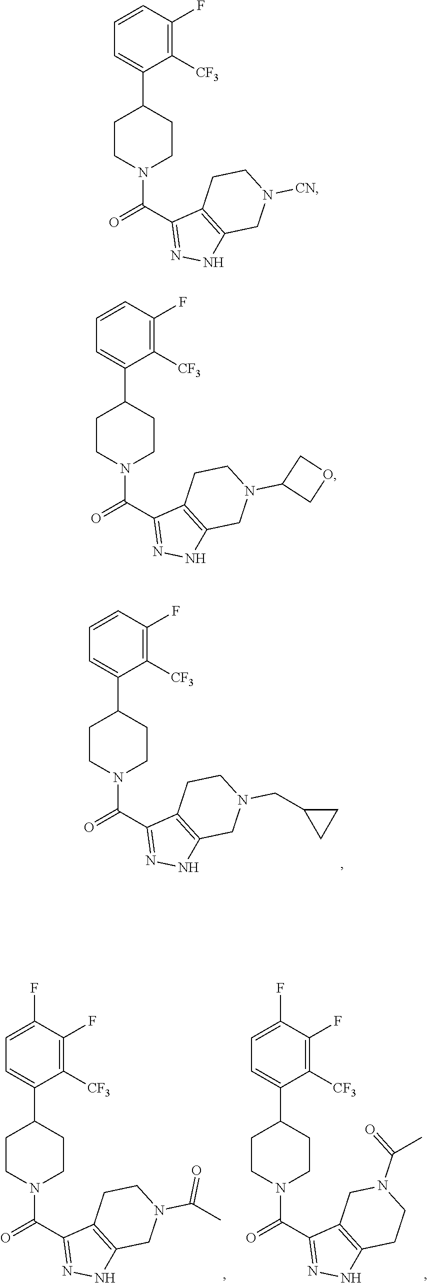

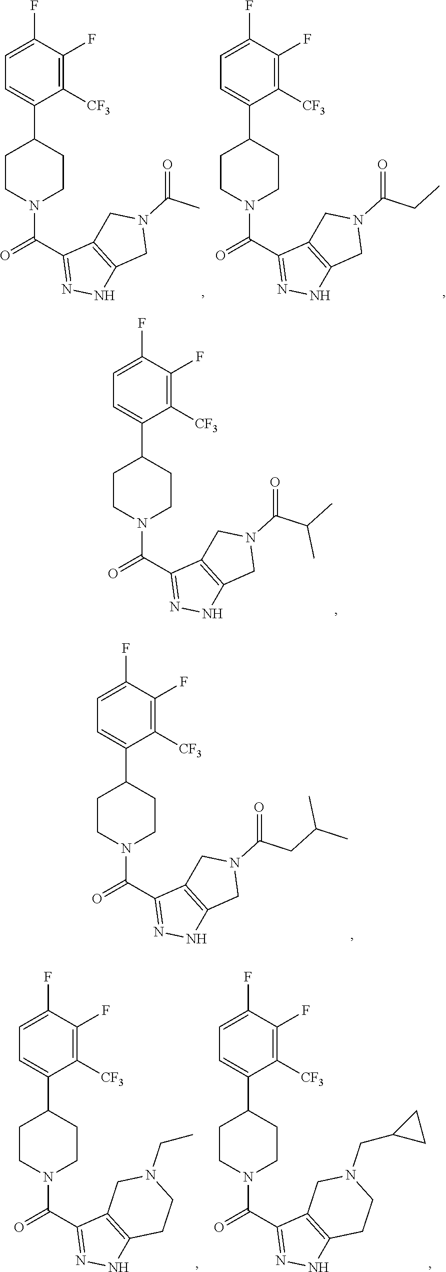

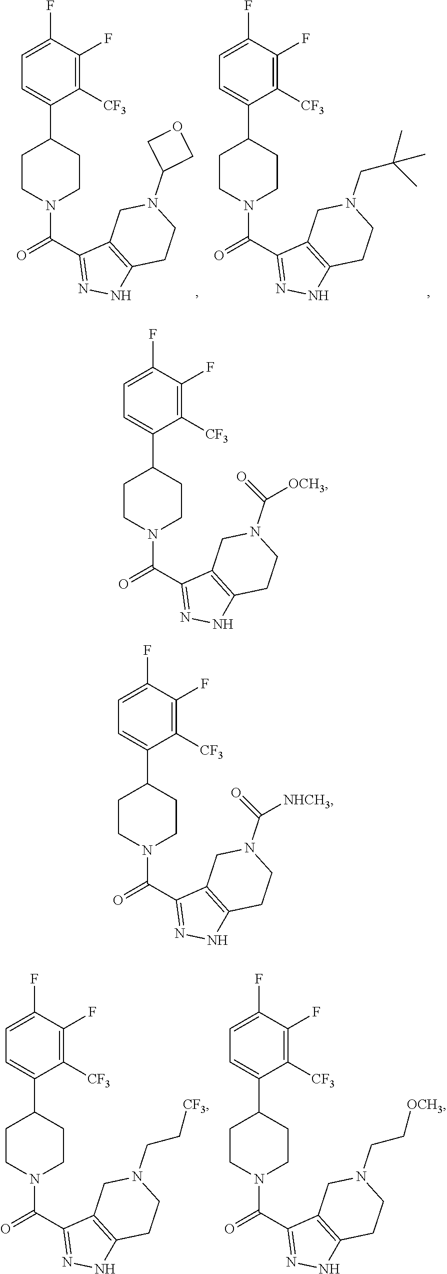

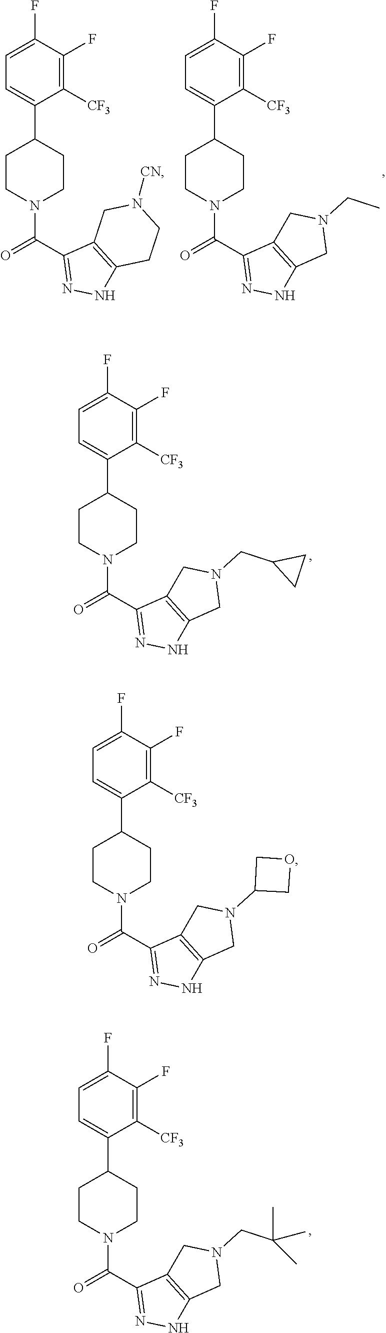

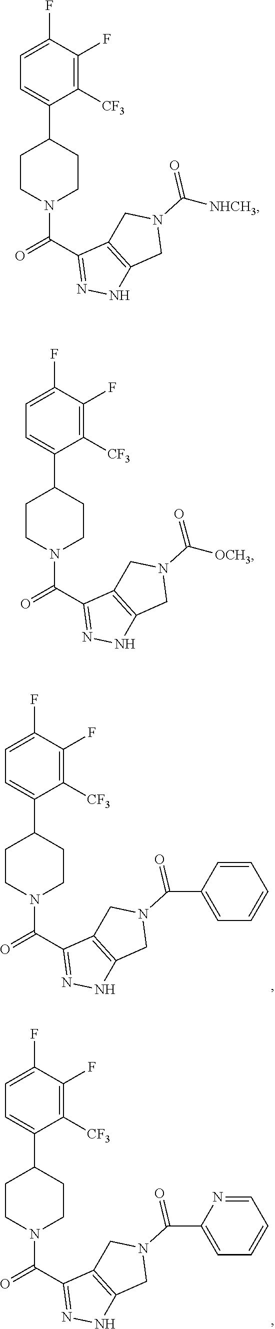 [SMILES: CNC(=O)N1Cc2[nH]nc(C(=O)N3CCC(c4ccc(F)c(F)c4C(F)(F)F)CC3)c2C1 ; COC(=O)N1Cc2[nH]nc(C(=O)N3CCC(c4ccc(F)c(F)c4C(F)(F)F)CC3)c2C1 ; O=C(c1ccccc1)N1Cc2[nH]nc(C(=O)N3CCC(c4ccc(F)c(F)c4C(F)(F)F)CC3)c2C1 ; O=C(c1ccccn1)N1Cc2[nH]nc(C(=O)N3CCC(c4ccc(F)c(F)c4C(F)(F)F)CC3)c2C1]

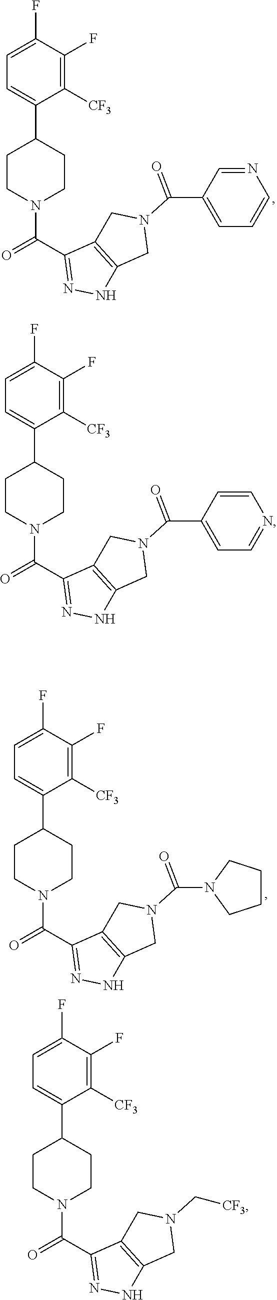

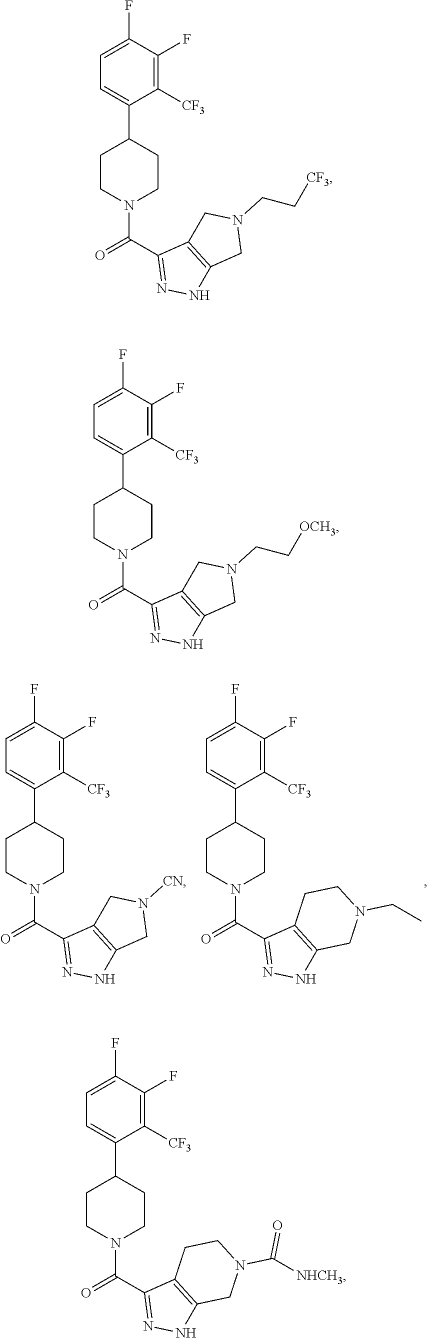

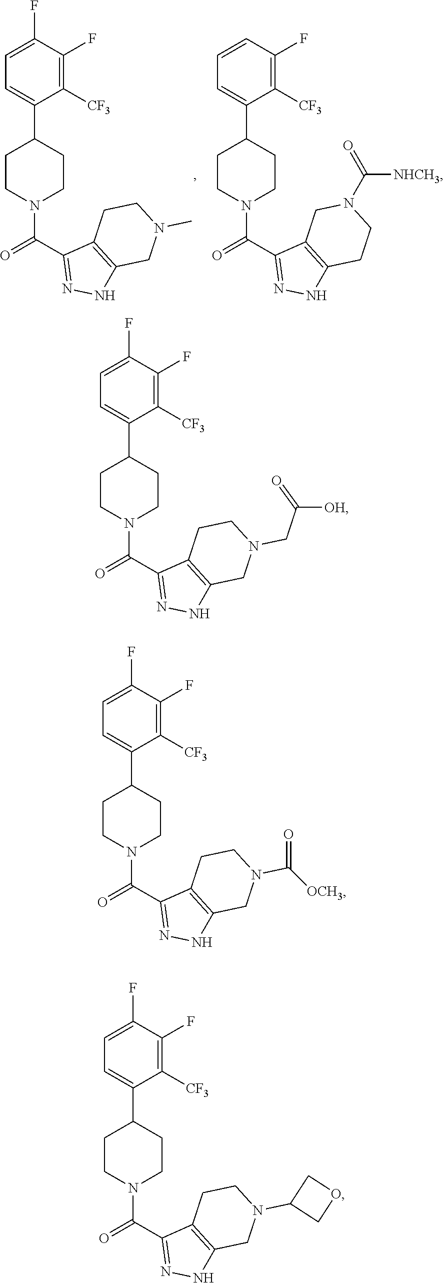

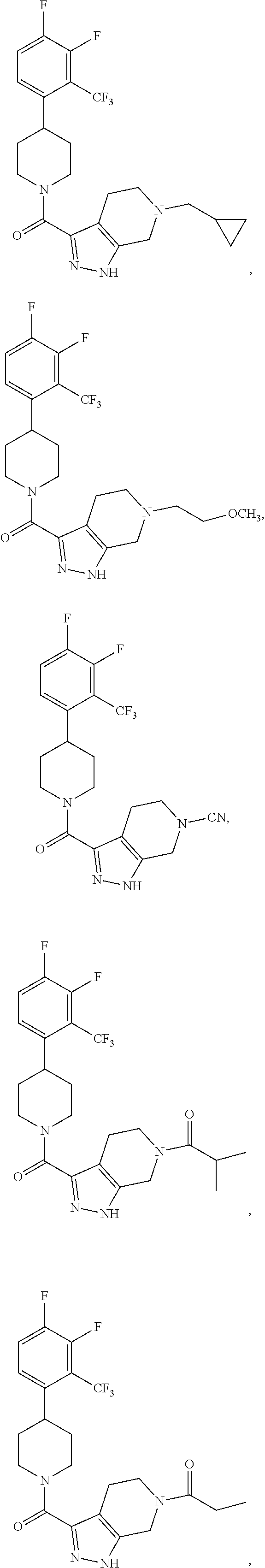 [SMILES: O=C(c1n[nH]c2c1CCN(CC1CC1)C2)N1CCC(c2ccc(F)c(F)c2C(F)(F)F)CC1 ; COCCN1CCc2c(C(=O)N3CCC(c4ccc(F)c(F)c4C(F)(F)F)CC3)n[nH]c2C1 ; N#CN1CCc2c(C(=O)N3CCC(c4ccc(F)c(F)c4C(F)(F)F)CC3)n[nH]c2C1 ; CC(C)C(=O)N1CCc2c(C(=O)N3CCC(c4ccc(F)c(F)c4C(F)(F)F)CC3)n[nH]c2C1 ; CCC(=O)N1CCc2c(C(=O)N3CCC(c4ccc(F)c(F)c4C(F)(F)F)CC3)n[nH]c2C1]

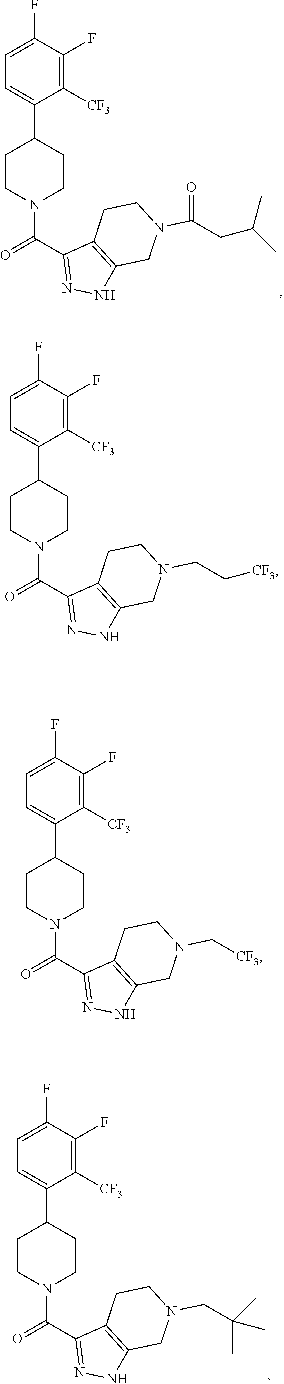 [SMILES: CC(C)CC(=O)N1CCc2c(C(=O)N3CCC(c4ccc(F)c(F)c4C(F)(F)F)CC3)n[nH]c2C1 ; O=C(c1n[nH]c2c1CCN(CCC(F)(F)F)C2)N1CCC(c2ccc(F)c(F)c2C(F)(F)F)CC1 ; O=C(c1n[nH]c2c1CCN(CC(F)(F)F)C2)N1CCC(c2ccc(F)c(F)c2C(F)(F)F)CC1 ; CC(C)(C)CN1CCc2c(C(=O)N3CCC(c4ccc(F)c(F)c4C(F)(F)F)CC3)n[nH]c2C1]

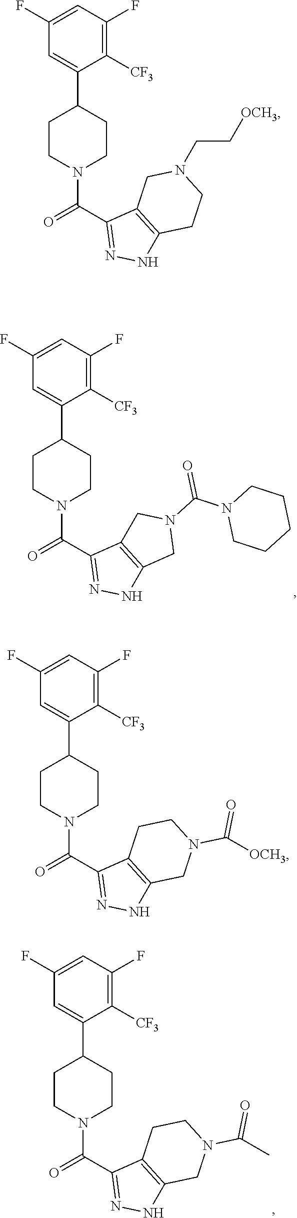

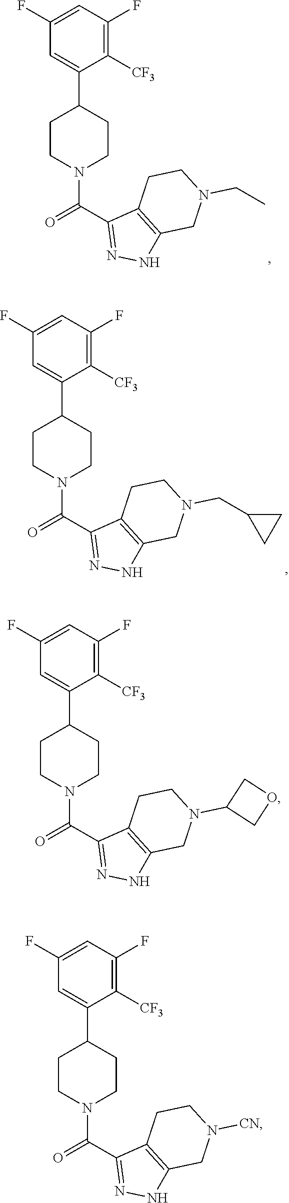 [SMILES: CCN1CCc2c(C(=O)N3CCC(c4cc(F)cc(F)c4C(F)(F)F)CC3)n[nH]c2C1 ; O=C(c1n[nH]c2c1CCN(CC1CC1)C2)N1CCC(c2cc(F)cc(F)c2C(F)(F)F)CC1 ; O=C(c1n[nH]c2c1CCN(C1COC1)C2)N1CCC(c2cc(F)cc(F)c2C(F)(F)F)CC1 ; N#CN1CCc2c(C(=O)N3CCC(c4cc(F)cc(F)c4C(F)(F)F)CC3)n[nH]c2C1]

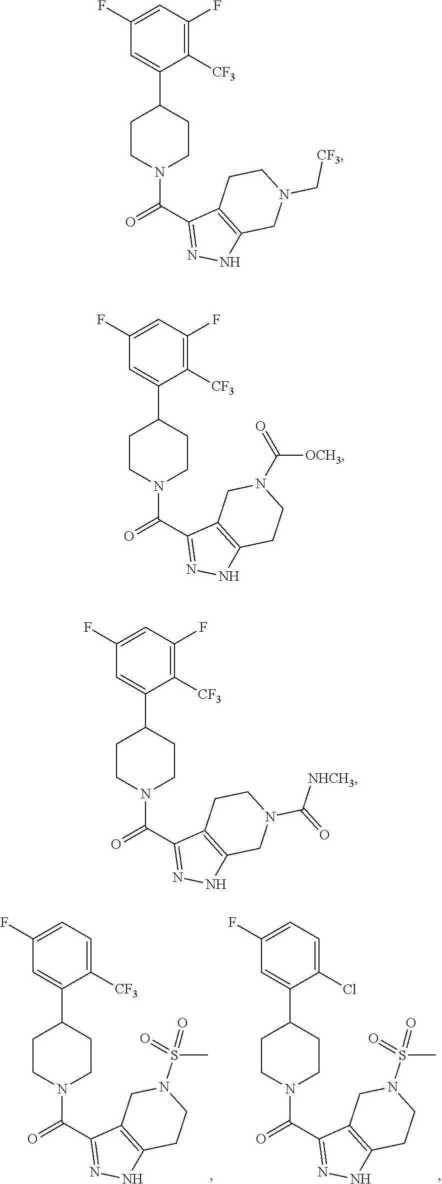

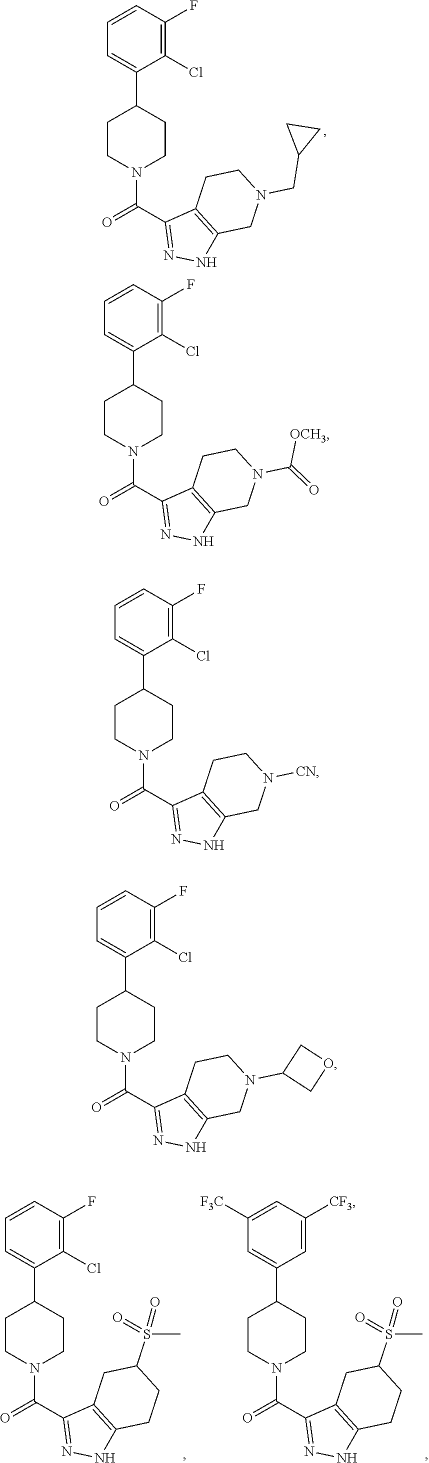

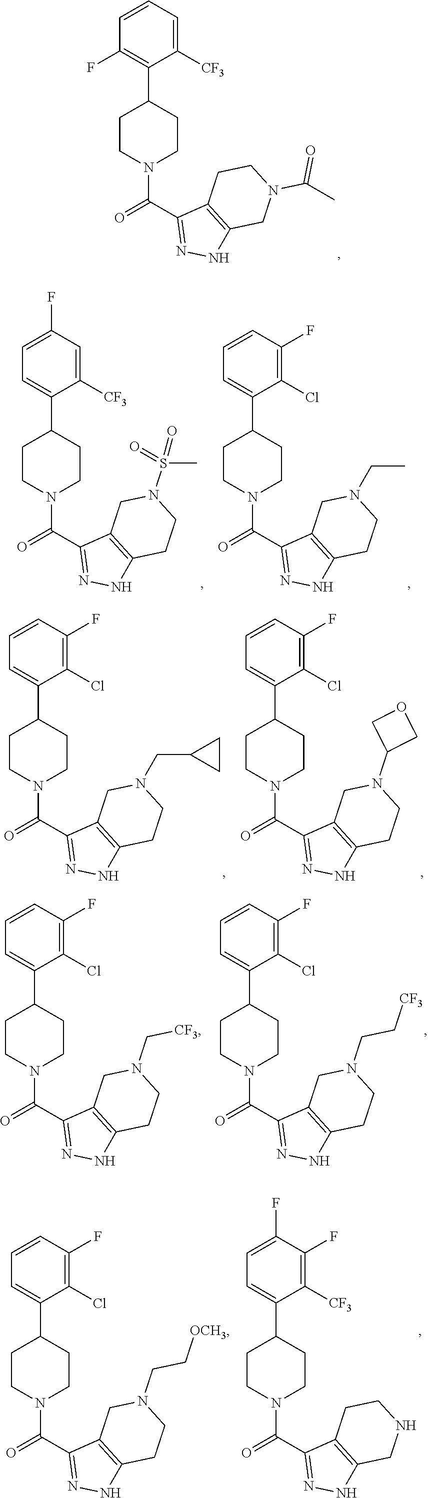

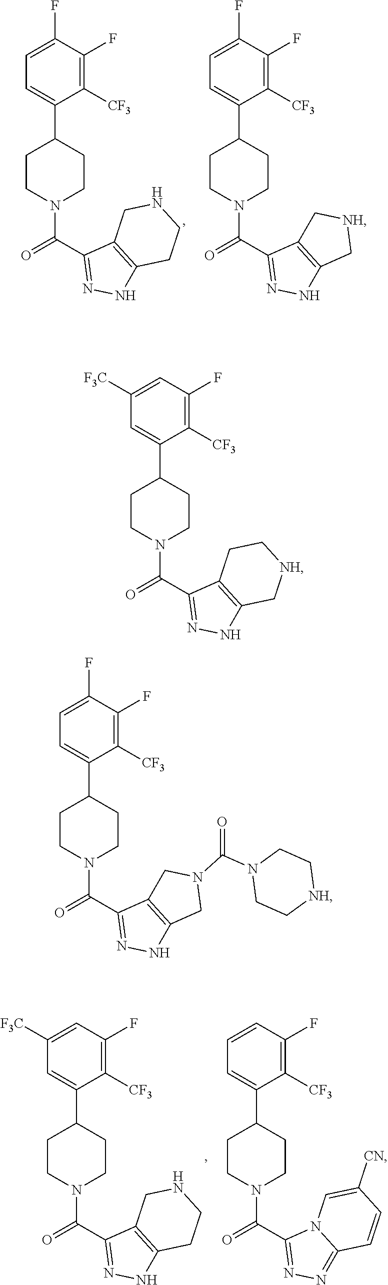

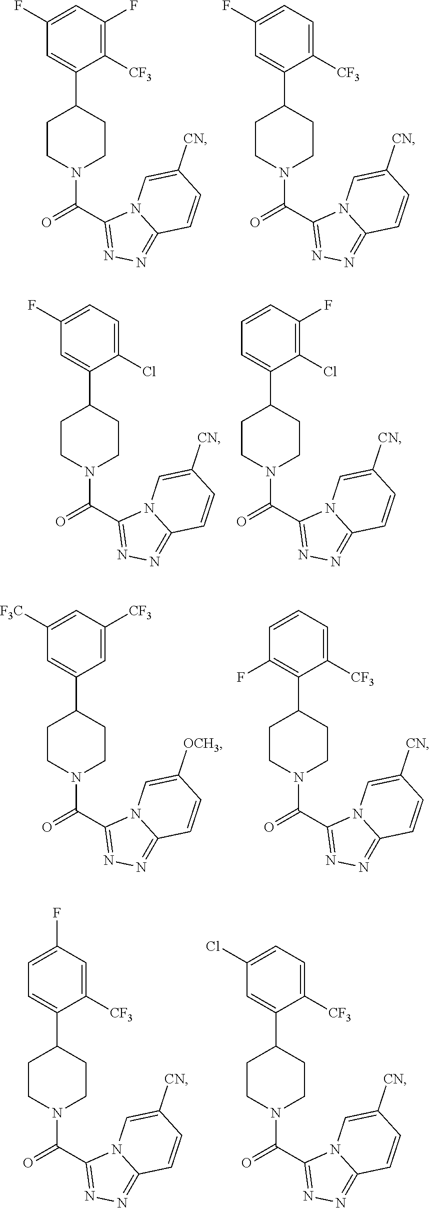

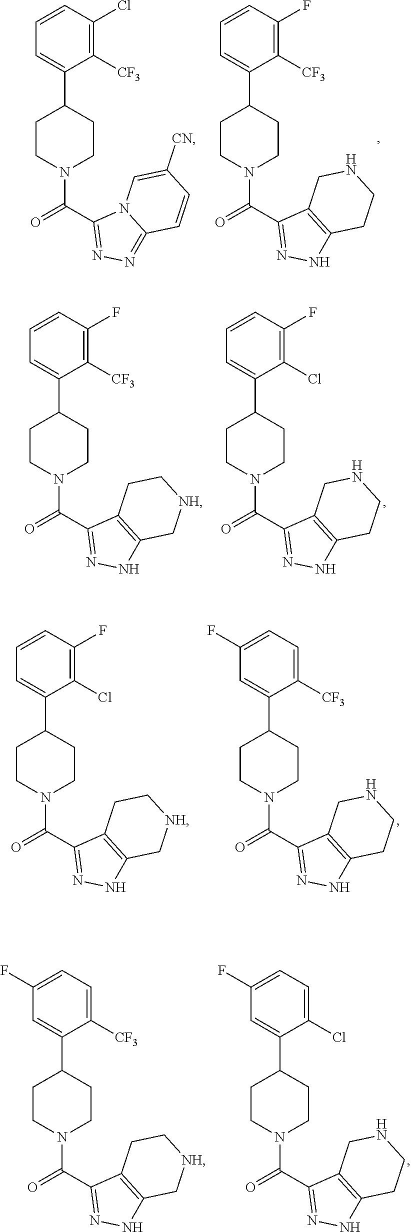

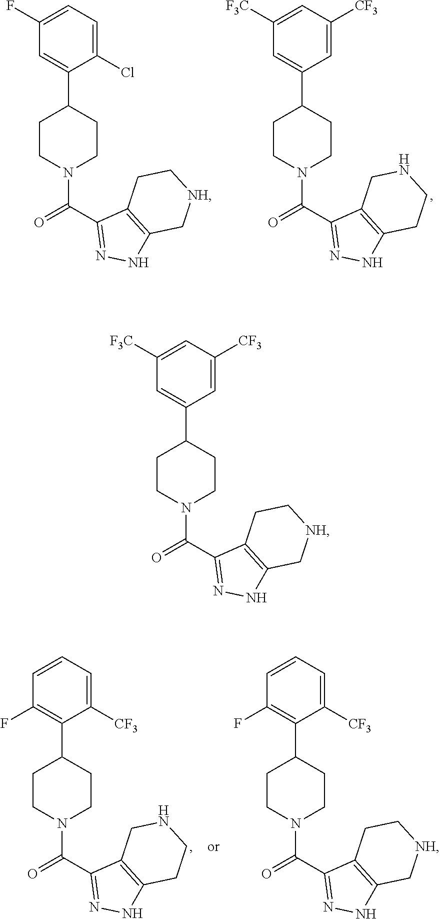 [SMILES: O=C(c1n[nH]c2c1CCNC2)N1CCC(c2cc(F)ccc2Cl)CC1 ; O=C(c1n[nH]c2c1CNCC2)N1CCC(c2cc(C(F)(F)F)cc(C(F)(F)F)c2)CC1 ; O=C(c1n[nH]c2c1CCNC2)N1CCC(c2cc(C(F)(F)F)cc(C(F)(F)F)c2)CC1 ; O=C(c1n[nH]c2c1CNCC2)N1CCC(c2c(F)cccc2C(F)(F)F)CC1 ; O=C(c1n[nH]c2c1CCNC2)N1CCC(c2c(F)cccc2C(F)(F)F)CC1]

or a pharmaceutically acceptable salt thereof.

In some embodiments, the selective RBP4 antagonist is

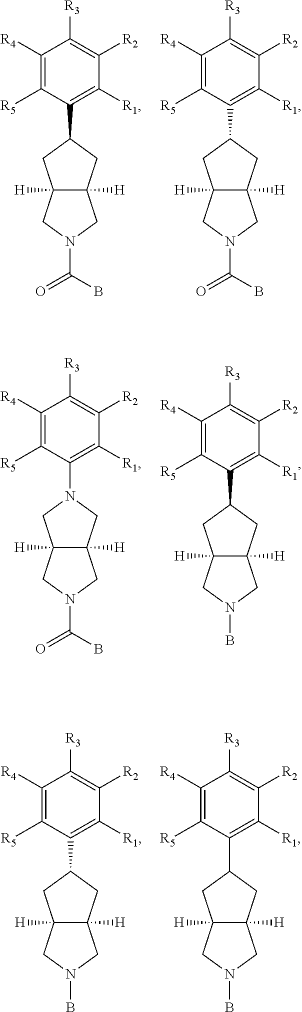

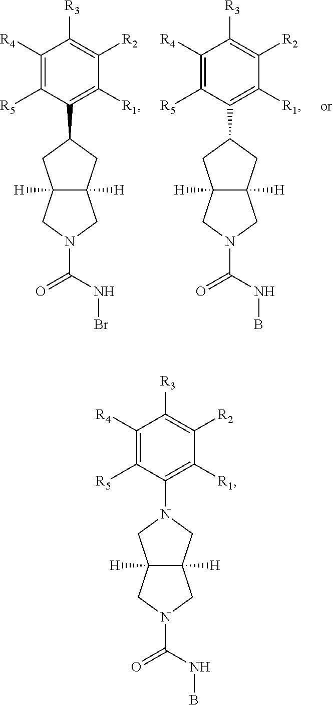

or a pharmaceutically acceptable salt thereof.

In some embodiments, the selective RBP4 antagonist is

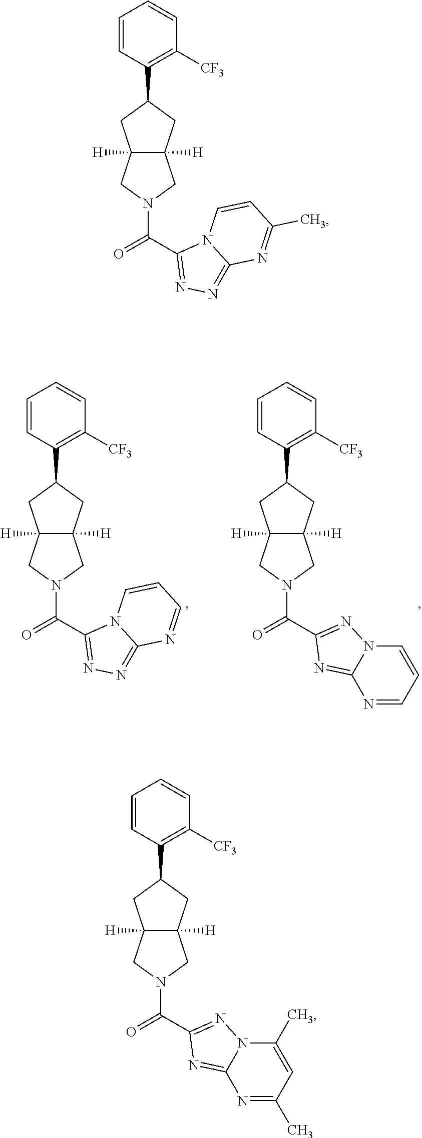 [SMILES: Cc1ccn2c(C(=O)N3C[C@H]4C[C@@H](c5ccccc5C(F)(F)F)C[C@H]4C3)nnc2n1 ; O=C(c1nnc2ncccn12)N1C[C@H]2C[C@@H](c3ccccc3C(F)(F)F)C[C@H]2C1 ; O=C(c1nc2ncccn2n1)N1C[C@H]2C[C@@H](c3ccccc3C(F)(F)F)C[C@H]2C1 ; Cc1cc(C)n2nc(C(=O)N3C[C@H]4C[C@@H](c5ccccc5C(F)(F)F)C[C@H]4C3)nc2n1]

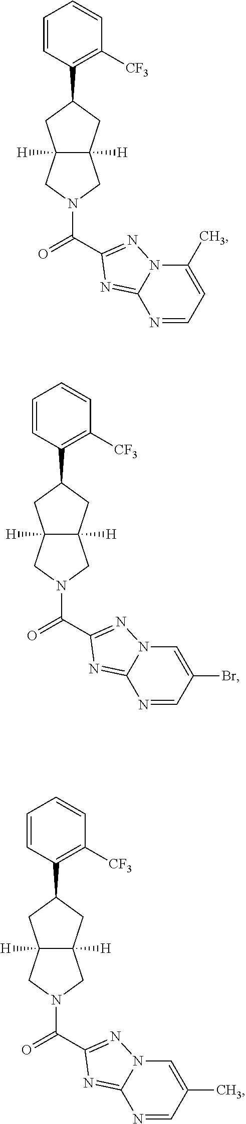 [SMILES: Cc1ccnc2nc(C(=O)N3C[C@H]4C[C@@H](c5ccccc5C(F)(F)F)C[C@H]4C3)nn12 ; O=C(c1nc2ncc(Br)cn2n1)N1C[C@H]2C[C@@H](c3ccccc3C(F)(F)F)C[C@H]2C1 ; Cc1cnc2nc(C(=O)N3C[C@H]4C[C@@H](c5ccccc5C(F)(F)F)C[C@H]4C3)nn2c1]

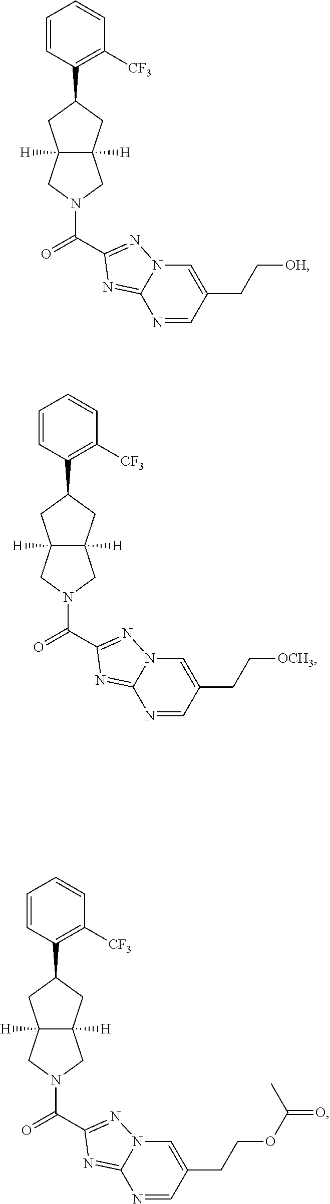 [SMILES: O=C(c1nc2ncc(CCO)cn2n1)N1C[C@H]2C[C@@H](c3ccccc3C(F)(F)F)C[C@H]2C1 ; COCCc1cnc2nc(C(=O)N3C[C@H]4C[C@@H](c5ccccc5C(F)(F)F)C[C@H]4C3)nn2c1 ; CC(=O)OCCc1cnc2nc(C(=O)N3C[C@H]4C[C@@H](c5ccccc5C(F)(F)F)C[C@H]4C3)nn2c1]

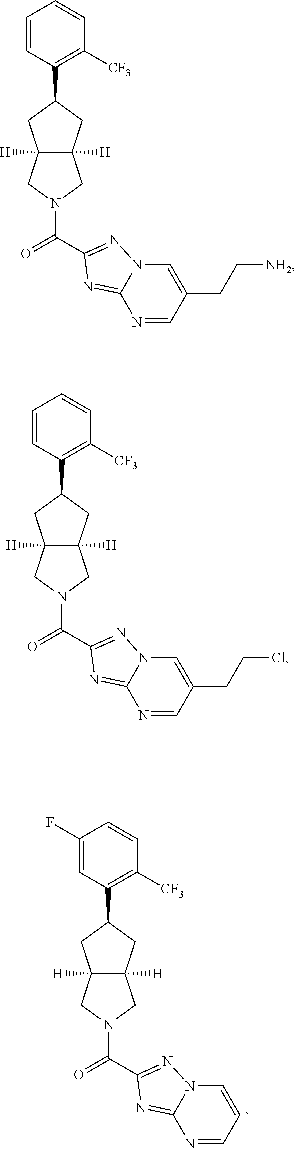 [SMILES: NCCc1cnc2nc(C(=O)N3C[C@H]4C[C@@H](c5ccccc5C(F)(F)F)C[C@H]4C3)nn2c1 ; O=C(c1nc2ncc(CCCl)cn2n1)N1C[C@H]2C[C@@H](c3ccccc3C(F)(F)F)C[C@H]2C1 ; O=C(c1nc2ncccn2n1)N1C[C@H]2C[C@@H](c3cc(F)ccc3C(F)(F)F)C[C@H]2C1]

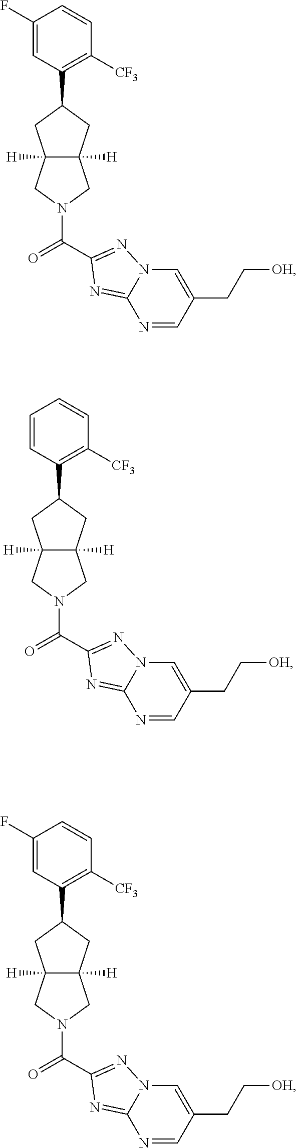 [SMILES: O=C(c1nc2ncc(CCO)cn2n1)N1C[C@H]2C[C@@H](c3cc(F)ccc3C(F)(F)F)C[C@H]2C1 ; O=C(c1nc2ncc(CCO)cn2n1)N1C[C@H]2C[C@@H](c3ccccc3C(F)(F)F)C[C@H]2C1 ; O=C(c1nc2ncc(CCO)cn2n1)N1C[C@H]2C[C@@H](c3cc(F)ccc3C(F)(F)F)C[C@H]2C1]

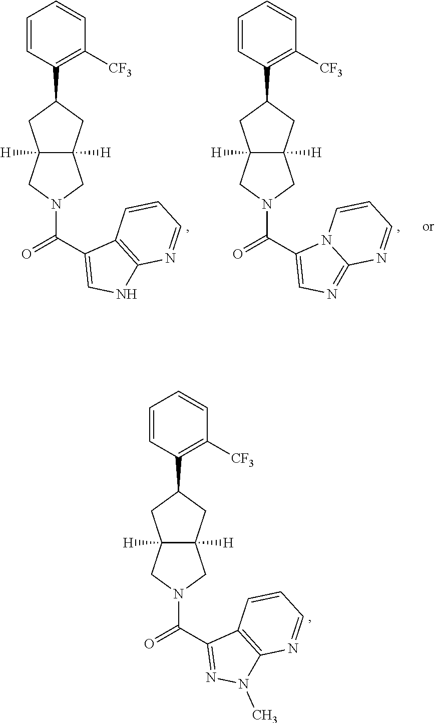 [SMILES: O=C(c1c[nH]c2ncccc12)N1C[C@H]2C[C@@H](c3ccccc3C(F)(F)F)C[C@H]2C1 ; O=C(c1cnc2ncccn12)N1C[C@H]2C[C@@H](c3ccccc3C(F)(F)F)C[C@H]2C1 ; Cn1nc(C(=O)N2C[C@H]3C[C@@H](c4ccccc4C(F)(F)F)C[C@H]3C2)c2cccnc21]

or a pharmaceutically acceptable salt thereof.

In some embodiments, the selective RBP4 antagonist is the compound having the structure

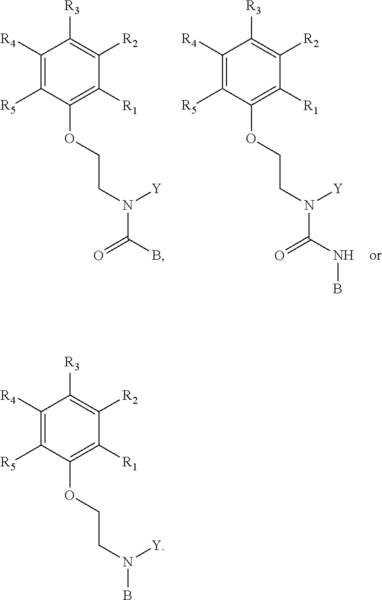

or a pharmaceutically acceptable salt thereof.

In some embodiments, the selective RBP4 antagonist is

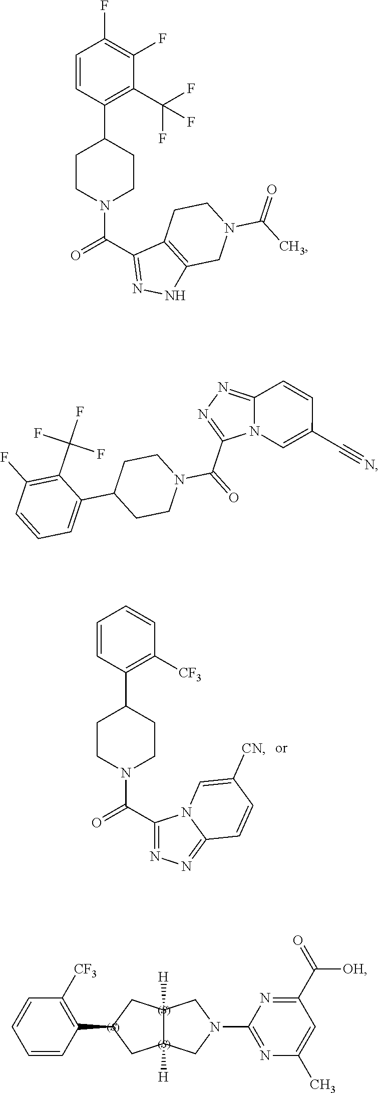

or a pharmaceutically acceptable salt thereof.

In some embodiments, the selective RBP4 antagonist is the compound having the structure:

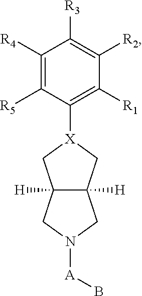

wherein
R₁, R₂, R₃, R₄, and R₅ are each independently H, halogen, CF₃, or C₁-C₄ alkyl;
X is N or CR₆,
   wherein R₆ is H, OH, or halogen;
A is absent or present, and when present, is

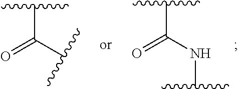

B has the structure:

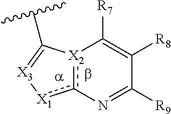

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

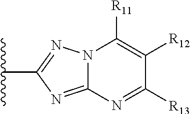

wherein
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound having the structure

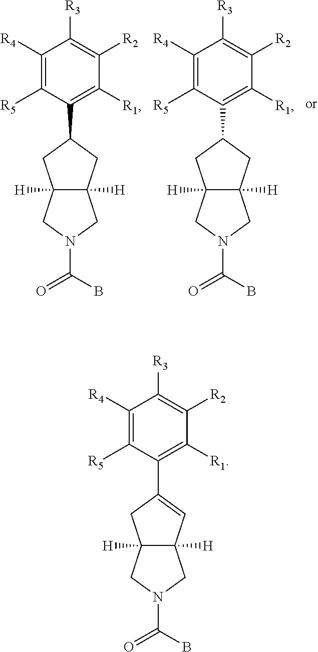

In some embodiments, the selective RBP4 antagonist is the compound having the structure

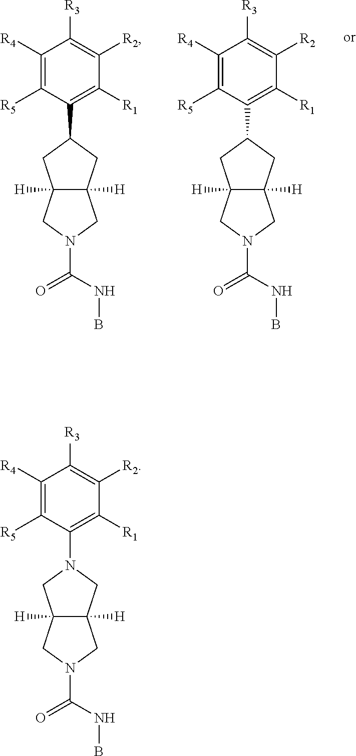

In some embodiments, the selective RBP4 antagonist is the compound having the structure

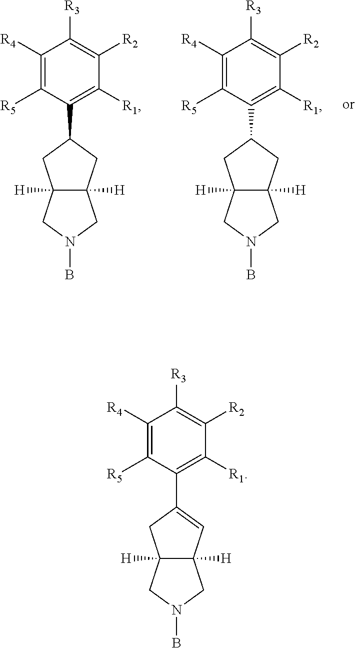

In some embodiments, the selective RBP4 antagonist is the compound having the structure:

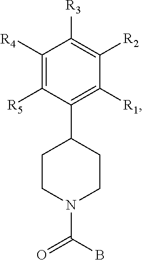

wherein
R₁, R₂, R₃, R₄, and R₅ are each independently H, halogen, CF₃, or C₁-C₄ alkyl; and
B has the structure:

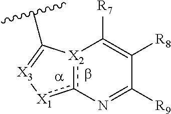

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

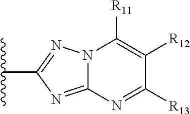

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
   or a pharmaceutically acceptable salt thereof.

In some embodiments, the selective RBP4 antagonist is the compound having the structure:

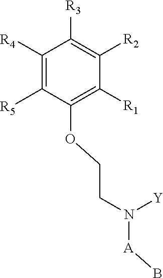

wherein
R₁, R₂, R₃, R₄, and R₅ are each independently H, halogen, CF₃, or C₁-C₄ alkyl;
Y is alkyl;
A is absent or present, and when present, is

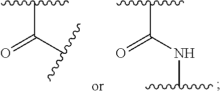

and
B has the structure:

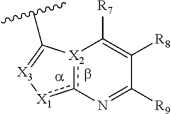

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

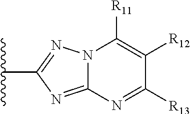

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
or a pharmaceutically acceptable salt thereof.

In some embodiments, the selective RBP4 antagonist is the compound having the structure

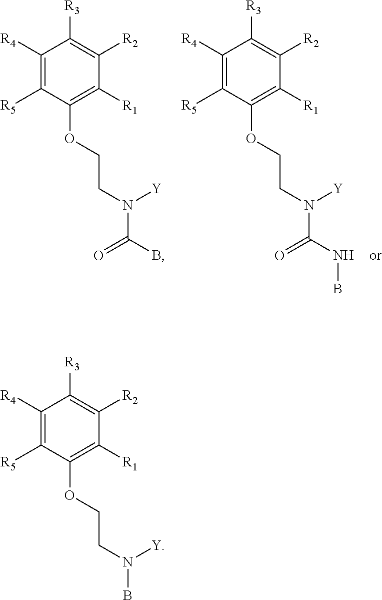

In some embodiments, the selective RBP4 antagonist is the compound wherein B has the structure:

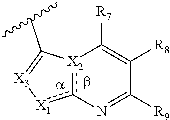

wherein
α and β are each a bond that is present or absent;
X₁ is N, NH, or NR₁₀,
   wherein R₁₀ is alkyl, alkenyl, or alkynyl;
X₂ is C or N;
X₃ is CH or N; and
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H.

In some embodiments, the selective RBP4 antagonist is the compound wherein B or B₂ has the structure

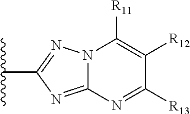

wherein
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein B or B₂ has the structure

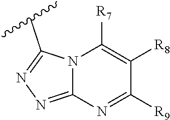

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein B or B₂ has the structure

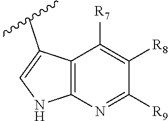

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein B or B₂ has the structure

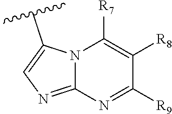

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein B or B₂ has the structure:

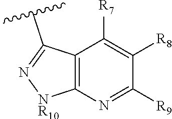

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃; and
R₁₀ is alkyl, alkenyl, or alkynyl.

In some embodiments, the selective RBP4 antagonist is the compound wherein R₇, R₈, and R₉ are each independently H, Cl, Br, F, OCH₃, OCH₂CH₃, CF₃, CN, CH₃, CH₂CH₃, C(O)OH, or C(O)—NH₂.

In some embodiments, the selective RBP4 antagonist is the compound wherein R₇, R₈, and R₉ are each independently H, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein R₇, R₈, and R₉ are each independently H, halogen, or alkyl.

In some embodiments, the selective RBP4 antagonist is the compound wherein two of R₇, R₈, and R₉ are each H and the remaining one of R₇, R₈, and R₉ is other than H.

In some embodiments, the selective RBP4 antagonist is the compound wherein one of R₇, R₈, and R₉ is H and the remaining two of R₇, R₈, and R₉ are each other than H.

In some embodiments, the selective RBP4 antagonist is the compound wherein B or B₂ has the structure

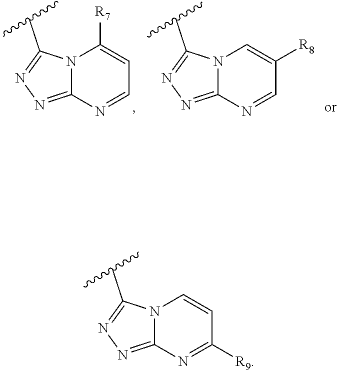

In some embodiments, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

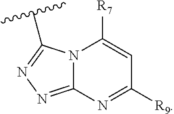

In some embodiments, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

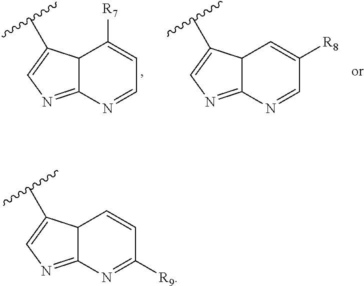

In some embodiments, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

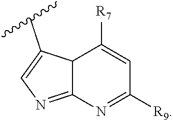

In some embodiments, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B has the structure

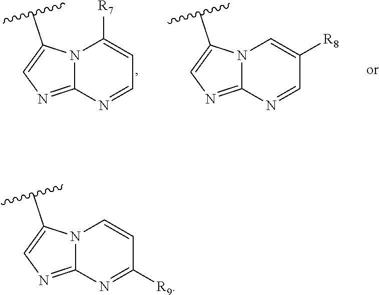

In some embodiments, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

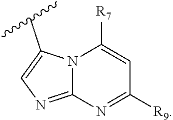

In some embodiments, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

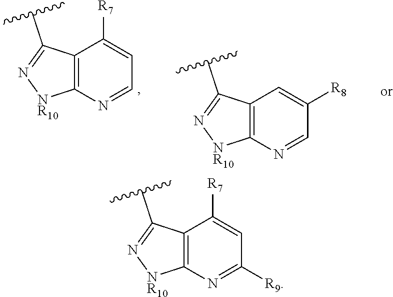

In some embodiments, the selective RBP4 antagonist is the compound wherein:
R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br; and
R₁₀ is alkyl.

In some embodiments, the selective RBP4 antagonist is the compound wherein B or B₂ has the structure

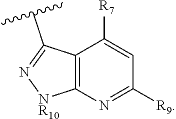

In some embodiments, the selective RBP4 antagonist is the compound wherein:
R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br; and
R₁₀ is alkyl.

In some embodiments, the selective RBP4 antagonist is the compound wherein B or B₂ has the structure

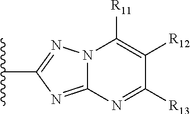

wherein R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments, the selective RBP4 antagonist is the compound wherein R₁₁, R₁₂, and R₁₃ are each independently H, Cl, Br, F, OCH₃, OCH₂CH₃, CF₃, CN, CH₃, CH₂CH₃, C(O)OH, or C(O)—NH₂.

In some embodiments, the selective RBP4 antagonist is the compound wherein R₁₁, R₁₂, and R₁₃ are each independently H, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein R₁₁, R₁₂, and R₁₃ are each independently H, halogen, or alkyl.

In some embodiments, the selective RBP4 antagonist is the compound wherein two of R₁₁, R₁₂, and R₁₃ are each H and the remaining one of R₁₁, R₁₂, and R₁₃ is other than H.

In some embodiments, the selective RBP4 antagonist is the compound wherein one of R₁₁, R₁₂, and R₁₃ is H and the remaining two of R₁₁, R₁₂, and R₁₃ are each other than H.

In some embodiments, the selective RBP4 antagonist is the compound wherein B or B₂ has the structure

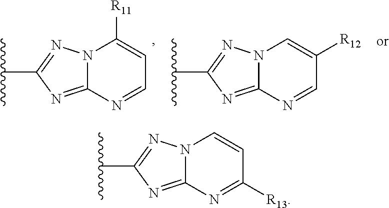

In some embodiments, the selective RBP4 antagonist is the compound wherein R₁₁, R₁₂, and R₁₃ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein B or B₂ has the structure

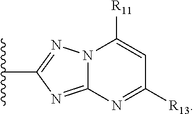

In some embodiments, the selective RBP4 antagonist is the compound wherein R₁₁ and R₁₃ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments, the selective RBP4 antagonist is the compound wherein X is N.

In some embodiments, the selective RBP4 antagonist is the compound wherein X is CH.

In some embodiments, the selective RBP4 antagonist is the compound wherein R₁, R₂, R₃, R₄, and R₅ are each H, t-Bu, Cl, F, or CF₃.

In some embodiments, the selective RBP4 antagonist is the compound wherein:
R₁, R₂, R₃, and R₄ are each H; and
R₅ is CF₃ or t-Bu.

In some embodiments, the selective RBP4 antagonist is the compound wherein:
R₁, R₃, and R₄ are each H;
R₂ is halogen; and
R₅ is CF₃ or t-Bu.

In some embodiments, the selective RBP4 antagonist is the compound wherein:
R₁, R₂, R₃, and R₄ are each H; and
R₅ is CF₃ or t-Bu.

In some embodiments, the selective RBP4 antagonist is the compound wherein:
R₁, R₂, R₃, and R₄ are each H; and
R₅ is CF₃.

In some embodiments, the selective RBP4 antagonist is the compound wherein one of R₁, R₂, R₃, R₄, and R₅ is other than H.

In some embodiments, the selective RBP4 antagonist is the compound wherein two of R₁, R₂, R₃, R₄, and R₅ are other than H.

In some embodiments, the selective RBP4 antagonist is the compound wherein two or more of R₁, R₂, R₃, R₄, and R₅ are other than H.

In some embodiments, the selective RBP4 antagonist is the compound wherein three of R₁, R₂, R₃, R₄, and R₅ are other than H.

In some embodiments, the selective RBP4 antagonist is the compound wherein three or more of R₁, R₂, R₃, R₄, and R₅ are other than H.

In some embodiments, the selective RBP4 antagonist is

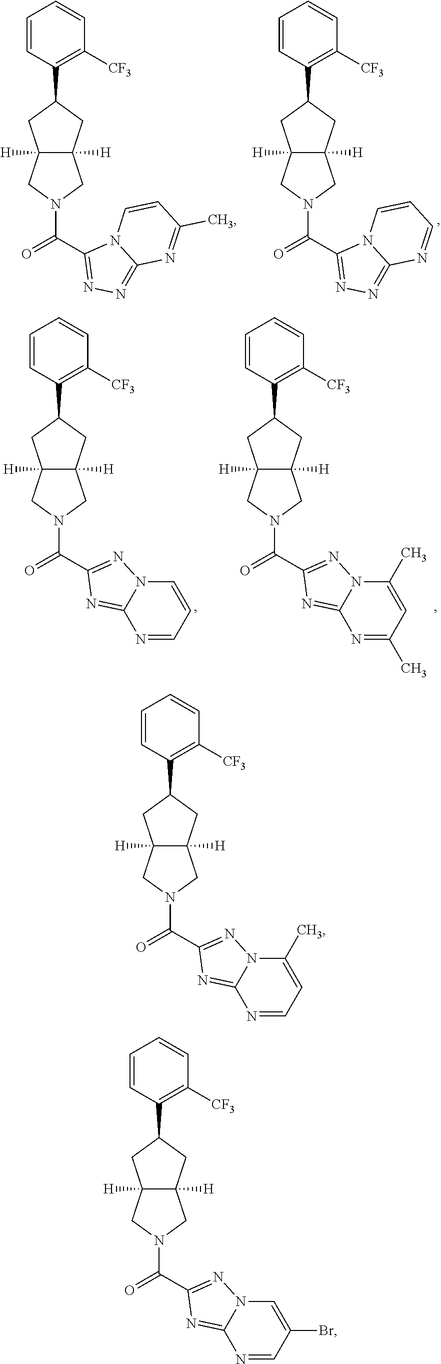

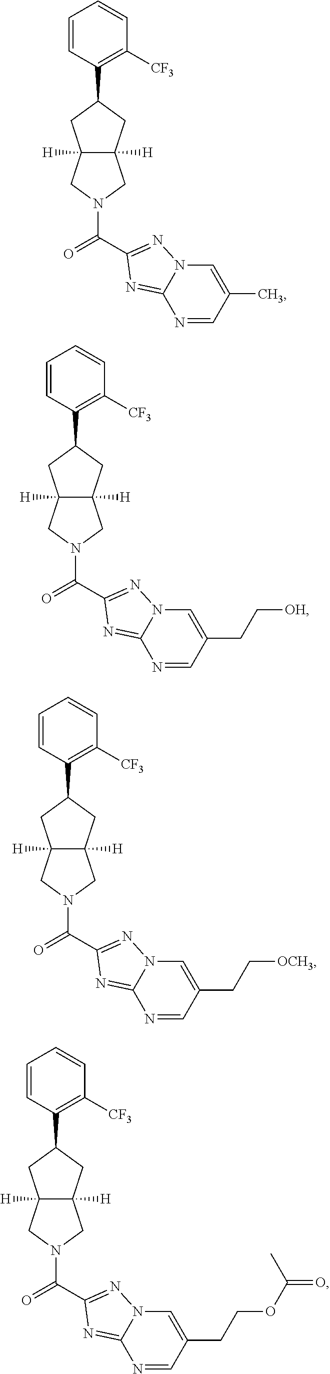 [SMILES: Cc1cnc2nc(C(=O)N3C[C@H]4C[C@@H](c5ccccc5C(F)(F)F)C[C@H]4C3)nn2c1 ; O=C(c1nc2ncc(CCO)cn2n1)N1C[C@H]2C[C@@H](c3ccccc3C(F)(F)F)C[C@H]2C1 ; COCCc1cnc2nc(C(=O)N3C[C@H]4C[C@@H](c5ccccc5C(F)(F)F)C[C@H]4C3)nn2c1 ; CC(=O)OCCc1cnc2nc(C(=O)N3C[C@H]4C[C@@H](c5ccccc5C(F)(F)F)C[C@H]4C3)nn2c1]

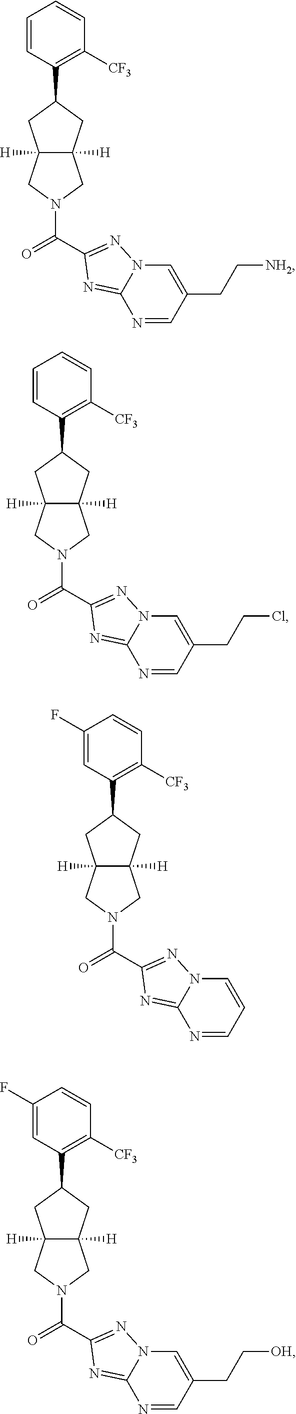 [SMILES: NCCc1cnc2nc(C(=O)N3C[C@H]4C[C@@H](c5ccccc5C(F)(F)F)C[C@H]4C3)nn2c1 ; O=C(c1nc2ncc(CCCl)cn2n1)N1C[C@H]2C[C@@H](c3ccccc3C(F)(F)F)C[C@H]2C1 ; O=C(c1nc2ncccn2n1)N1C[C@H]2C[C@@H](c3cc(F)ccc3C(F)(F)F)C[C@H]2C1 ; O=C(c1nc2ncc(CCO)cn2n1)N1C[C@H]2C[C@@H](c3cc(F)ccc3C(F)(F)F)C[C@H]2C1]

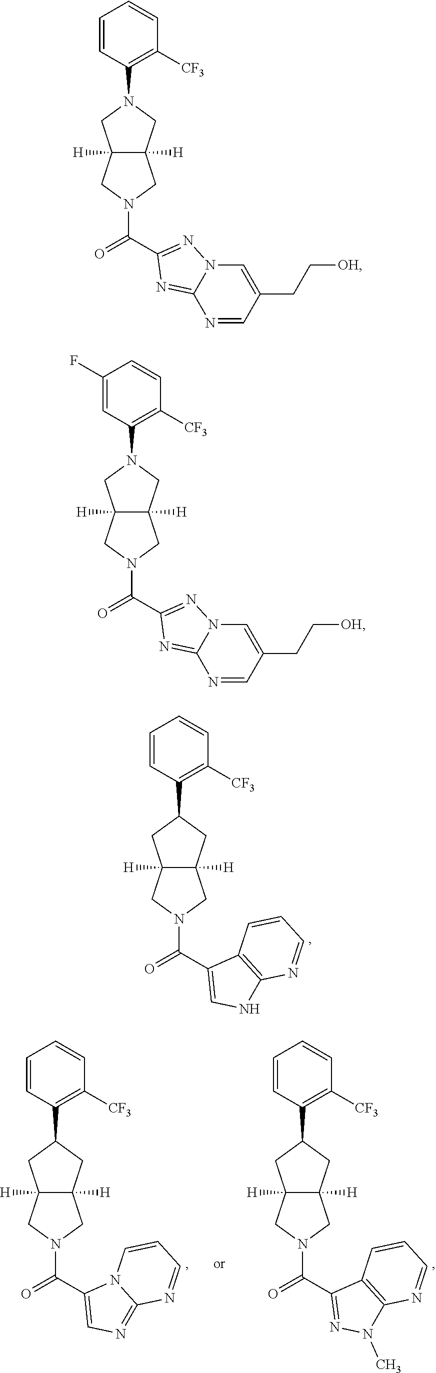

or a pharmaceutically acceptable salt thereof.

In some embodiments, the selective RBP4 antagonist is the compound wherein B or B₂ is other than

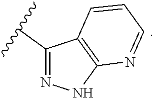 [SMILES: *c1n[nH]c2ncccc12]

In some embodiments, the selective RBP4 antagonist is the compound having the structure:

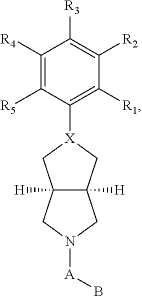

wherein
R₁, R₂, R₃, R₄, and R₅ are each independently H, halogen, alkyl, haloalkyl, O-haloalkyl, aryl, or heteroaryl;
X is N or CR₆,
   wherein R₆ is H, OH, or halogen;
A is absent or present, and when present, is

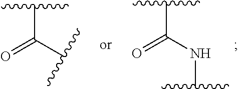

and
B has the structure:

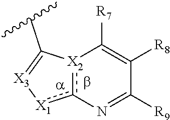

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

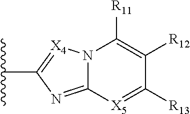

wherein
X₄ and X₅ are each independently N or CH; and
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound having the structure:

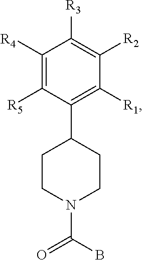

wherein
R₁, R₂, R₃, R₄, and R₅ are each independently H, halogen, CF₃, C₁-C₄ alkyl, aryl, or heteroaryl; and
B has the structure:

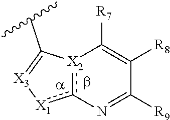

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

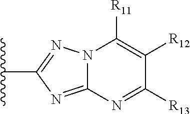

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound having the structure:

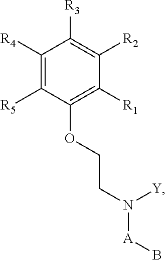

wherein
R₁, R₂, R₃, R₄, and R₅ are each independently H, halogen, CF₃, C₁-C₄ alkyl, aryl, or heteroaryl;
Y is alkyl;
A is absent or present, and when present, is

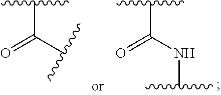

and
B has the structure:

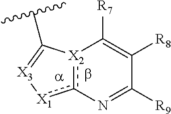

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

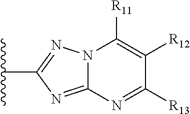

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments, the selective RBP4 antagonist is the compound wherein one of R₁, R₂, R₃, R₄, and R₅ is other than H.

In some embodiments, the selective RBP4 antagonist is the compound wherein two of R₁, R₂, R₃, R₄, and R₅ are other than H.

In some embodiments, the selective RBP4 antagonist is the compound wherein R₁, R₂, R₃, R₄, and R₅ are each H, methyl, ethyl, phenyl, t-Bu, i-Pr, OCF₃, CF₃, OCF₂CF₃, CF₂CF₃, Cl, Br, or F.

In some embodiments, the selective RBP4 antagonist is the compound wherein:
R₁, R₂, R₃, and R₄ are each H; and
R₅ is —H, OCF₃, CF₂CF₃, methyl, ethyl, i-Pr, or phenyl.

In some embodiments, the selective RPB4 antagonist is

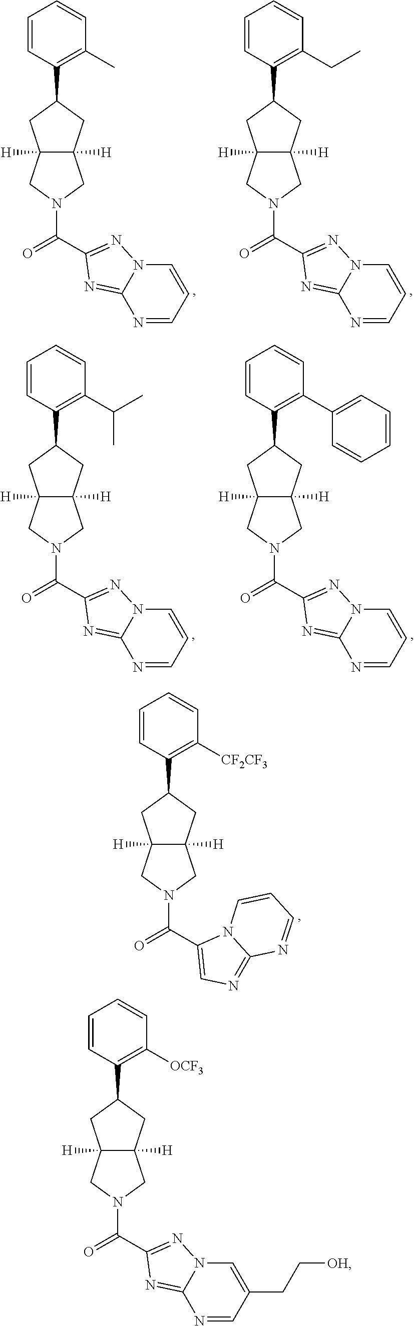

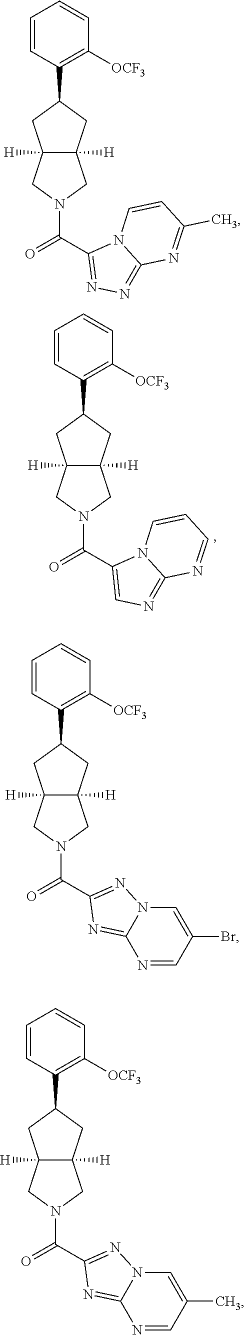

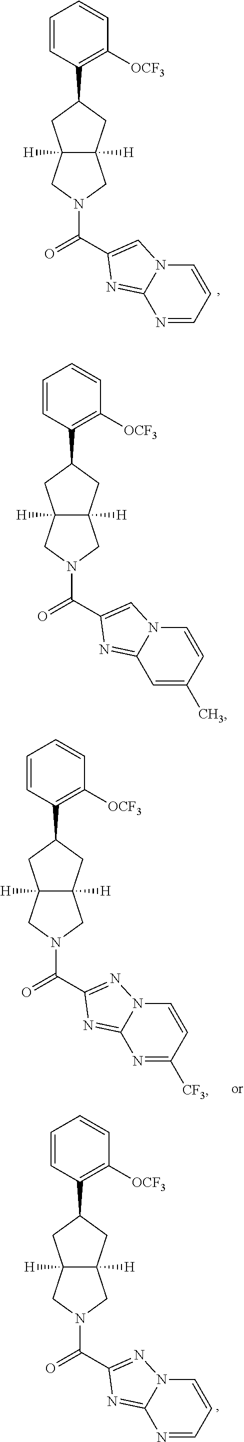 [SMILES: O=C(c1cn2cccnc2n1)N1C[C@H]2C[C@@H](c3ccccc3OC(F)(F)F)C[C@H]2C1 ; Cc1ccn2cc(C(=O)N3C[C@H]4C[C@@H](c5ccccc5OC(F)(F)F)C[C@H]4C3)nc2c1 ; O=C(c1nc2nc(C(F)(F)F)ccn2n1)N1C[C@H]2C[C@@H](c3ccccc3OC(F)(F)F)C[C@H]2C1 ; "or" ; O=C(c1nc2ncccn2n1)N1C[C@H]2C[C@@H](c3ccccc3OC(F)(F)F)C[C@H]2C1]

or a pharmaceutically acceptable salt thereof.

In some embodiments, the C20-D3-visual-chromophore-producing compound is in a form packaged in chylomicrons.

The present disclosure provides a method for treating a disease characterized by excessive or age-related lipofuscin accumulation in the retina in a mammal afflicted therewith, and for promoting rhodopsin and cone opsins production, comprising administering to the mammal an effective amount of the pharmaceutical composition of the present disclosure for a simultaneous, contemporaneous, or concomitant administration of the selective RBP4 antagonist and the C20-D3-visual-chromaphore-producing compound.

In some embodiments of the method, the disease is further characterized by bisretinoid-mediated macular degeneration.

In some embodiments of the method, the amount of the selective RBP4 antagonist is effective to lower the serum concentration of RBP4 in the mammal, or the amount of the selective RBP4 antagonist is effective to lower the retinal concentration of a bisretinoid in lipofuscin in the mammal.

In some embodiments of the method, the bisretinoid is A2E.

In some embodiments of the method, the bisretinoid is isoA2E.

In some embodiments of the method, the bisretinoid is A2-DHP-PE.

In some embodiments of the method, the bisretinoid is atRAL di-PE.

In some embodiments of the method, the disease characterized by age-related lipofuscin accumulation in the retina is Age-Related Macular Degeneration.

In some embodiments of the method, the disease characterized by age-related lipofuscin accumulation in the retina is dry (atrophic) Age-Related Macular Degeneration.

In some embodiments of the method, the disease characterized by excessive lipofuscin accumulation in the retina is Stargardt Disease.

In some embodiments of the method, the disease characterized by excessive lipofuscin accumulation in the retina is other forms of retinopathy caused by or associated with mutations in the ABCA4 gene, such as retinitis pigmentosa (RP19) or cone-rod dystrophy (CORD3).

In some embodiments of the method, the disease characterized by excessive lipofuscin accumulation in the retina is Best disease.

In some embodiments of the method, the disease characterized by excessive lipofuscin accumulation in the retina is adult vitelliform maculopathy.

In some embodiments of the method, the disease characterized by excessive lipofuscin accumulation in the retina is Stargardt-like macular dystrophy.

In some embodiments of the method, the administration is effective to reduce photoreceptor degeneration.

In some embodiments of the method, the bisretinoid-mediated macular degeneration is Age-Related Macular Degeneration or Stargardt Disease.

In some embodiments of the method, the bisretinoid-mediated macular degeneration is Age-Related Macular Degeneration.

In some embodiments of the method, the bisretinoid-mediated macular degeneration is dry (atrophic) Age-Related Macular Degeneration.

In some embodiments of the method, the bisretinoid-mediated macular degeneration is Stargardt Disease.

In some embodiments of the method, the bisretinoid-mediated macular degeneration is other forms of retinopathy caused by or associated with mutations in the ABCA4 gene, such as retinitis pigmentosa (RP19) or cone-rod dystrophy (CORD3).

In some embodiments of the method, the bisretinoid-mediated macular degeneration is Best disease.

In some embodiments of the method, the bisretinoid-mediated macular degeneration is adult vitelliform maculopathy.

In some embodiments of the method, the bisretinoid-mediated macular degeneration is Stargardt-like macular dystrophy.

In some embodiments, the pharmaceutical composition of the present disclosure exhibits retinol-binding protein 4 (RBP4) antagonist activity as well as activity of promoting rhodopsin and cone opsins production.

In some embodiments, the pharmaceutical composition of the present disclosure reduces circulating RBP4 levels and promotes rhodopsin and cone opsins production.

In some embodiments, the pharmaceutical composition of the present disclosure may be used for the treatment of dry age-related macular degeneration (AMD).

In some embodiments, the pharmaceutical composition of the present disclosure may be used for the treatment of type 2 diabetes.

In some embodiments, the pharmaceutical composition of the present disclosure may be used for the treatment of obesity.

In some embodiments, the pharmaceutical composition of the present disclosure may be used for the treatment of insulin resistance.

In some embodiments, the pharmaceutical composition of the present disclosure may be used for the treatment of cardiovascular disease.

In some embodiments, the pharmaceutical composition of the present disclosure may be used for the treatment of hepatic steatosis.

In some embodiments, the pharmaceutical composition of the present disclosure may be used for the treatment of non-alcoholic fatty liver disease (NAFLD).

The present disclosure provides a method for treating a non-alcoholic fatty liver disease (NAFLD) in a mammal afflicted therewith, and for promoting rhodopsin and cone opsins production, comprising administering to the mammal an effective amount of the pharmaceutical composition of the present disclosure for a simultaneous, contemporaneous, or concomitant administration of the selective RBP4 antagonist and the C20-D3-visual-chromophore-producing compound.

The present disclosure provides a method for treating gout in a mammal afflicted therewith, and for promoting rhodopsin and cone opsins production, comprising administering to the mammal an effective amount of the pharmaceutical composition of the present disclosure for a simultaneous, contemporaneous, or concomitant administration of the selective RBP4 antagonist and the C20-D3-visual-chromophore-producing compound.

In some embodiments of the method, the mammal is afflicted with a NAFLD selected from the group consisting of hepatic steatosis (fatty liver), non-alcoholic steatohepatitis (NASH), cirrhosis, and hepatocellular carcinoma.

In some embodiments of the method, the method further comprises a step of determining, or having determined, the level of RBP4 in adipose tissue in the mammal and administering to the mammal the pharmaceutical composition of the present disclosure if the level of RBP4 in adipose tissue is elevated.

In some embodiments of the method, the method further comprises a step of determining, or having determined, the level of RBP4 in serum in the mammal and administering to the mammal the pharmaceutical composition of the present disclosure if the level of RBP4 in serum is elevated.

In some embodiments of the method, the amount of the selective RBP4 antagonist is effective in reducing RBP4 levels in adipose tissue in the mammal.

In some embodiments of the method, the amount of the selective RBP4 antagonist is effective in reducing RBP4 levels in serum in the mammal.

In some embodiments of the method, the amount of the selective RBP4 antagonist is effective in reducing uric acid levels in the serum of the mammal.

In some embodiments of the method, the amount of the selective RBP4 antagonist is effective to normalize the concentration of triglycerides in the liver of the mammal.

In some embodiments of the method, the amount of the selective RBP4 antagonist is effective to normalize the concentration of free fatty acids in the serum of the mammal.

In some embodiments of the method, the amount of the selective RBP4 antagonist is effective to normalize the concentration of free fatty acids in the liver of the mammal.

In some embodiments of the method, the amount of the selective RBP4 antagonist is effective to prevent trafficking of a fatty acid by RBP4.

In some embodiments of the method, the amount of the selective RBP4 antagonist is effective to prevent trafficking of a fatty acid to the liver by RBP4.

In some embodiments of the method, the amount of the selective RBP4 antagonist is effective to inhibit binding between RBP4 and a fatty acid.

In some embodiments of the method, the fatty acid is from adipose tissue.

In some embodiments of the method, the mammal does not have elevated serum RBP4 levels.

In some embodiments of the method, the mammal has elevated serum RBP4 levels.

In some embodiments of the method, the serum RBP4 level of the mammal is elevated by more than 3 micrograms per ml.

In some embodiments of the method, the NAFLD is a hepatic steatosis selected from simple hepatic steatosis and mild hepatic steatosis.

In some embodiments of the method, the amount of the selective RBP4 antagonist is 5-1000 mg, 5-800 mg, 5-200 mg, 45-200 mg, 45-1000 mg, 45-800 mg, 10-50 mg, 96 mg, 24 mg, or 10 mg per day.

In some embodiments of the method, the method further comprises administering an amount of a second agent which is (R)-(+)-(5,6-dichloro 2,3,9,9a-tetrahydro 3-oxo-9a-propyl-1H-fluoren-7-yl)oxy]acetic acid (DPOFA), a Nonsteroidal Anti-inflammatory Drug (NSAID) such as indomethacin, colchicine, lesinurad, corticosteroids (e.g., betamethasone, prednisone, dexamethasone, cortisone, hydrocortisone, methylprednisone, prednisolone), biologic anti-IL-1alpha/beta agents (e.g., canakinumab, rilonacept, anakinra), allopurinol, benzbromarone, pegloticase, and other forms of uricase enzymes, topiroxostat (FYX-051), ulodesine (BCX4208), KUX-1151, RLBN1001, RDEA3170, arhalofenate (MBX-102), levotofisopam, UR-1102, PF-06743649, BCX4208, SHR4640, Lumiracoxib, Tranilast, Topiroxostat, LC350189, Bucillamine, AC-201, HuZhen Capsules (including Polygonum cuspidatum and Ligustrum lucidum), MPC-004, FYU-981, Sodium Bicarbonate, SEL-212, SEL-037, Apremilast, TMX-67, SSS11, D-0120, febuxostat, or probenecid, or esters or salts thereof, effective to treat the mammal.

In some embodiments of the method, the second agent is DPOFA.

In some embodiments of the method, the mammal is afflicted with gout.

In some embodiments of the method, the amount of the second agent and/or the amount of the selective RBP4 antagonist is effective in reducing uric acid levels in the blood of the mammal.

In some embodiments of the method, the amount of the second agent and/or the amount of the selective RBP4 antagonist is effective in decreasing uric acid reabsorption in the kidneys of the mammal.

In some embodiments of the method, the amount of the second agent is effective in increasing uric acid clearance in the mammal.

In some embodiments of the method, the amount of the second agent is effective in increasing uric acid levels in the urine of the mammal.

In some embodiments of the method, the amount of the second agent is effective in increasing renal clearance of uric acid in the mammal.

In some embodiments of the method, the amount of the second agent and/or the amount of the selective RBP4 antagonist is effective in reducing one or more symptoms associated with gout in the mammal.

In some embodiments of the method, the one or more symptoms associated with gout are selected from joint pain, joint inflammation, joint redness, and decreased range of motion at the joint.

In some embodiments of the method, the amount of the second agent and/or the amount of the selective RBP4 antagonist is effective in preventing gout in the mammal.

In some embodiments of the method, the preventing comprises increasing uric acid levels in the urine of the mammal.

In some embodiments of the method, the preventing comprises reducing uric acid levels in the blood of the mammal.

In some embodiments of the method, the preventing comprises increasing uric acid clearance in the mammal.

In some embodiments of the method, the preventing comprises decreasing uric acid reabsorption in the kidneys of the mammal.

In some embodiments of the method, the preventing comprises increasing renal clearance of uric acid in the mammal.

In some embodiments of the method, the preventing comprises reducing one or more symptoms associated with gout in the mammal.

In some embodiments of the method, the one or more symptoms associated with gout are selected from joint pain, joint inflammation, joint redness, and decreased range of motion at the joint.

In some embodiments of the method, the gout is chronic gout.

In some embodiments of the method, the gout is acute gout.

In some embodiments of the method, the amount of the second agent and/or the amount of the selective RBP4 antagonist prevents a recurrence of chronic gout.

In some embodiments of the method, the mammal is female and the administration of the selective RBP4 antagonist reduces the uric acid level to 2.4-6.0 mg/dL.

In some embodiments of the method, the mammal is male and the administration of the selective RBP4 antagonist reduces the uric acid level to 3.4-7.0 mg/dL.

In some embodiments of the method, the administration of the selective RBP4 antagonist reduces uric acid levels in the mammal to less than 7 mg/dL.

In some embodiments of the method, the pharmaceutical composition of the present disclosure and the second agent are administered sequentially, simultaneously, contemporaneously, or concomitantly, in which in a sequential administration, the pharmaceutical composition of the present disclosure may be administered first or the second agent may be administered first.

The present disclosure provides the pharmaceutical composition of the present disclosure for use in a combination therapy together with a pharmaceutical composition comprising the second agent, for the treatment of a non-alcoholic fatty liver disease (NAFLD) or gout.

The present disclosure provides the pharmaceutical composition of the present disclosure comprising an amount of the selective RBP4 antagonist for use in treating a mammal afflicted with a non-alcoholic fatty liver disease (NAFLD) or gout as an add-on therapy to or in combination with the second agent.

In some embodiments of the method, the administration is oral.

In some embodiments, the mammal is a human.

The present disclosure provides a method for treating a disease characterized by excessive or age-related lipofuscin accumulation in the retina in a mammal afflicted therewith, and for promoting rhodopsin and cone opsins production, comprising the steps of sequentially, simultaneously, contemporaneously, or concomitantly administering to the mammal an amount of a selective RBP4 antagonist effective to treat a disease characterized by excessive or age-related lipofuscin accumulation in the retina and an amount of a C20-D3-visual-L-chromophore-producing compound effective to promote rhodopsin and cone opsins production, in which in a sequential administration, the selective RBP4 antagonist may be administered first or the C20-D3-visual-chromophore-producing compound may be administered first.

In some embodiments of the method, the disease is further characterized by bisretinoid-mediated macular degeneration.

In some embodiments of the method, the amount of the selective RBP4 antagonist is effective to lower the serum concentration of RBP4 in the mammal, or the amount of the selective RBP4 antagonist is effective to lower the retinal concentration of a bisretinoid in lipofuscin in the mammal.

In some embodiments of the method, the bisretinoid is A2E.

In some embodiments of the method, the bisretinoid is isoA2E.

In some embodiments of the method, the bisretinoid is A2-DHP-PE.

In some embodiments of the method, the bisretinoid is atRAL di-PE.

In some embodiments of the method, the disease characterized by age-related lipofuscin accumulation in the retina is Age-Related Macular Degeneration.

In some embodiments of the method, the disease characterized by age-related lipofuscin accumulation in the retina is dry (atrophic) Age-Related Macular Degeneration.

In some embodiments of the method, the disease characterized by excessive lipofuscin accumulation in the retina is Stargardt Disease.

In some embodiments of the method, the disease characterized by excessive lipofuscin accumulation in the retina is other forms of retinopathy caused by or associated with mutations in the ABCA4 gene, such as retinitis pigmentosa (RP19) or cone-rod dystrophy (CORD3).

In some embodiments of the method, the disease characterized by excessive lipofuscin accumulation in the retina is Best disease.

In some embodiments of the method, the disease characterized by excessive lipofuscin accumulation in the retina is adult vitelliform maculopathy.

In some embodiments of the method, the disease characterized by excessive lipofuscin accumulation in the retina is Stargardt-like macular dystrophy.

In some embodiments of the method, the administration is effective to reduce photoreceptor degeneration.

In some embodiments of the method, the bisretinoid-mediated macular degeneration is Age-Related Macular Degeneration or Stargardt Disease.

In some embodiments of the method, the bisretinoid-mediated macular degeneration is Age-Related Macular Degeneration.

In some embodiments of the method, the bisretinoid-mediated macular degeneration is dry (atrophic) Age-Related Macular Degeneration.

In some embodiments of the method, the bisretinoid-mediated macular degeneration is Stargardt Disease.

In some embodiments of the method, the bisretinoid-mediated macular degeneration is other forms of retinopathy caused by or associated with mutations in the ABCA4 gene, such as retinitis pigmentosa (RP19) or cone-rod dystrophy (CORD3).

In some embodiments of the method, the bisretinoid-mediated macular degeneration is Best disease.

In some embodiments of the method, the bisretinoid-mediated macular degeneration is adult vitelliform maculopathy.

In some embodiments of the method, the bisretinoid-mediated macular degeneration is Stargardt-like macular dystrophy.

The present disclosure provides a method for treating a non-alcoholic fatty liver disease (NAFLD) in a mammal afflicted therewith, and for promoting rhodopsin and cone opsins production, comprising the steps of sequentially, simultaneously, contemporaneously, or concomitantly administering to the mammal an amount of a selective RBP4 antagonist effective to treat a non-alcoholic fatty liver disease (NAFLD) and an amount of a C20-D3-visual-chromophore-producing compound effective to promote rhodopsin and cone opsins production, in which in a sequential administration, the selective RBP4 antagonist may be administered first or the C20-D3-visual-chromophore-producing compound may be administered first.

The present disclosure provides a method for treating gout in a mammal afflicted therewith, and for promoting rhodopsin and cone opsins production, comprising the steps of sequentially, simultaneously, contemporaneously, or concomitantly administering to the mammal an amount of a selective RBP4 antagonist effective to treat gout and an amount of a C20-D3-visual-chromophore-producing compound effective to promote rhodopsin and cone opsins production, in which in a sequential administration, the selective RBP4 antagonist may be administered first or the C20-D3-visual-chromophore-producing compound may be administered first.

In some embodiments of the method, the mammal is afflicted with a NAFLD selected from the group consisting of hepatic steatosis (fatty liver), non-alcoholic steatohepatitis (NASH), cirrhosis, and hepatocellular carcinoma.

In some embodiments of the method, the method further comprises a step of determining, or having determined, the level of RBP4 in adipose tissue in the mammal and administering to the mammal the selective RBP4 antagonist and the C20-D3-visual-chromophore-producing compound if the level of RBP4 in adipose tissue is elevated.

In some embodiments of the method, the method further comprises a step of determining, or having determined, the level of RBP4 in serum in the mammal and administering to the mammal the selective RBP4 antagonist and the C20-D3-visual-chromophore-producing compound, if the level of RBP4 in serum is elevated.

In some embodiments of the method, the amount of the selective RBP4 antagonist is effective in reducing RBP4 levels in adipose tissue in the mammal.

In some embodiments of the method, the amount of the selective RBP4 antagonist is effective in reducing RBP4 levels in serum in the mammal.

In some embodiments of the method, the amount of the selective RBP4 antagonist is effective in reducing uric acid levels in the serum of the mammal.

In some embodiments of the method, the amount of the selective RBP4 antagonist is effective to normalize the concentration of triglycerides in the liver of the mammal.

In some embodiments of the method, the amount of the selective RBP4 antagonist is effective to normalize the concentration of free fatty acids in the serum of the mammal.

In some embodiments of the method, the amount of the selective RBP4 antagonist is effective to normalize the concentration of free fatty acids in the liver of the mammal.

In some embodiments of the method, the amount of the selective RBP4 antagonist is effective to prevent trafficking of a fatty acid by RBP4.

In some embodiments of the method, the amount of the selective RBP4 antagonist is effective to prevent trafficking of a fatty acid to the liver by RBP4.

In some embodiments of the method, the amount of the selective RBP4 antagonist is effective to inhibit binding between RBP4 and a fatty acid.

In some embodiments of the method, the fatty acid is from adipose tissue.

In some embodiments of the method, the mammal does not have elevated serum RBP4 levels.

In some embodiments of the method, the mammal has elevated serum RBP4 levels.

In some embodiments of the method, the serum RBP4 level of the mammal is elevated by more than 3 micrograms per ml.

In some embodiments of the method, the NAFLD is a hepatic steatosis selected from simple hepatic steatosis and mild hepatic steatosis.

In some embodiments of the method, the amount of the selective RBP4 antagonist administered is 5-1000 mg, 5-800 mg, 5-200 mg, 45-200 mg, 45-1000 mg, 45-800 mg, 10-50 mg, 96 mg, 24 mg, or 10 mg per day.

In some embodiments of the method, the method further comprises administering an amount of a second agent which is (R)-(+)-(5,6-dichloro 2,3,9,9a-tetrahydro 3-oxo-9a-propyl-1H-fluoren-7-yl)oxy]acetic acid (DPOFA), a Nonsteroidal Anti-inflammatory Drug (NSAID) such as indomethacin, colchicine, lesinurad, corticosteroids (e.g., betamethasone, prednisone, dexamethasone, cortisone, hydrocortisone, methylprednisone, prednisolone), biologic anti-IL-1alpha/beta agents (e.g., canakinumab, rilonacept, anakinra), allopurinol, benzbromarone, pegloticase, and other forms of uricase enzymes, topiroxostat (FYX-051), ulodesine (BCX4208), KUX-1151, RLBN1001, RDEA3170, arhalofenate (MBX-102), levotofisopam, UR-1102, PF-06743649, BCX4208, SHR4640, Lumiracoxib, Tranilast, Topiroxostat, LC350189, Bucillamine, AC-201, HuZhen Capsules (including Polygonum cuspidatum and Ligustrum lucidum), MPC-004, FYU-981, Sodium Bicarbonate, SEL-212, SEL-037, Apremilast, TMX-67, SSS11, D-0120, febuxostat, or probenecid, or esters or salts thereof, effective to treat the mammal.

In some embodiments of the method, the second agent is DPOFA.

In some embodiments of the method, the mammal is afflicted with gout.

In some embodiments of the method, the amount of the second agent and/or the amount of the selective RBP4 antagonist is effective in reducing uric acid levels in the blood of the mammal.

In some embodiments of the method, the amount of the second agent and/or the amount of the selective RBP4 antagonist is effective in decreasing uric acid reabsorption in the kidneys of the mammal.

In some embodiments of the method, the amount of the second agent is effective in increasing uric acid clearance in the mammal.

In some embodiments of the method, the amount of the second agent is effective in increasing uric acid levels in the urine of the mammal.

In some embodiments of the method, the amount of the second agent is effective in increasing renal clearance of uric acid in the mammal.

In some embodiments of the method, the amount of the second agent and/or the amount of the selective RBP4 antagonist is effective in reducing one or more symptoms associated with gout in the mammal.

In some embodiments of the method, the one or more symptoms associated with gout are selected from joint pain, joint inflammation, joint redness, and decreased range of motion at the joint.

In some embodiments of the method, the amount of the second agent and/or the amount of the selective RBP4 antagonist is effective in preventing gout in the mammal.

In some embodiments of the method, the preventing comprises increasing uric acid levels in the urine of the mammal.

In some embodiments of the method, the preventing comprises reducing uric acid levels in the blood of the mammal.

In some embodiments of the method, the preventing comprises increasing uric acid clearance in the mammal.

In some embodiments of the method, the preventing comprises decreasing uric acid reabsorption in the kidneys of the mammal.

In some embodiments of the method, the preventing comprises increasing renal clearance of uric acid in the mammal.

In some embodiments of the method, the preventing comprises reducing one or more symptoms associated with gout in the mammal.

In some embodiments of the method, the one or more symptoms associated with gout are selected from joint pain, joint inflammation, joint redness, and decreased range of motion at the joint.

In some embodiments of the method, the gout is chronic gout.

In some embodiments of the method, the gout is acute gout.

In some embodiments of the method, the amount of the second agent and/or the amount of the selective RBP4 antagonist prevents a recurrence of chronic gout.

In some embodiments of the method, the mammal is female and the administration of the selective RBP4 antagonist reduces the uric acid level to 2.4-6.0 mg/dL.

In some embodiments of the method, the mammal is male and the administration of the selective RBP4 antagonist reduces the uric acid level to 3.4-7.0 mg/dL.

In some embodiments of the method, the administration of the selective RBP4 antagonist reduces uric acid levels in the mammal to less than 7 mg/dL.

In some embodiments of the method, the selective RBP4 antagonist, the C20-D3-visual-chromophore-producing compound, and the second agent are administered sequentially, simultaneously, contemporaneously, or concomitantly, in which:
the selective RBP4 antagonist may be administered first, the C20-D3-visual-chromophore-producing compound may be administered second, and the second agent may be administered third, all sequentially; or
the selective RBP4 antagonist may be administered first, the second agent may be administered second, and the C20-D3-visual-chromophore-producing compound may be administered third, all sequentially; or
the C20-D3-visual-chromophore-producing compound may be administered first, the selective RBP4 antagonist may be administered second, and the second agent may be administered third, all sequentially; or
the C20-D3-visual-chromophore-producing compound may be administered first, the second agent may be administered second, and the selective RBP4 antagonist may be administered third, all sequentially; or
the second agent may be administered first, the selective RBP4 antagonist may be administered second, and the C20-D3-visual-chromophore-producing compound may be administered third, all sequentially; or
the second agent may be administered first, the C20-D3-visual-chromophore-producing compound may be administered second, and the selective RBP4 antagonist may be administered third, all sequentially; or
the selective RBP4 antagonist and the C20-D3-visual-chromophore-producing compound may be administered first simultaneously, contemporaneously, or concomitantly, and then the second agent may be administered sequentially second; or
the second agent may be administered sequentially first, and then the selective RBP4 antagonist and the C20-D3-visual-chromophore-producing compound may be administered second simultaneously, contemporaneously, or concomitantly; or
the selective RBP4 antagonist and the second agent may be administered first simultaneously, contemporaneously, or concomitantly, and then the C20-D3-visual-chromophore-producing compound may be administered sequentially second; or
the C20-D3-visual-chromophore-producing compound may be administered sequentially first, and then the selective RBP4 antagonist and the second agent may be administered second simultaneously, contemporaneously, or concomitantly; or
the C20-D3-visual-chromophore-producing compound and the second agent may be administered first simultaneously, contemporaneously, or concomitantly, and then the selective RBP4 antagonist may be administered sequentially second; or
the selective RBP4 antagonist may be administered sequentially first, and then the C20-D3-visual-chromophore-producing compound and the second agent may be administered second simultaneously, contemporaneously, or concomitantly; or
the selective RBP4 antagonist, the C20-D3-visual-chromophore-producing compound, and the second agent may be administered all simultaneously, contemporaneously, or concomitantly.

In some embodiments of the method, the administration is oral.

In some embodiments of the method, the mammal is a human.

In some embodiments of the method, the C20-D3-visual-chromophore-producing compound is selected from the group consisting of C20-D3-retinol, C20-D3-retinaldehyde, C20-D3-retinyl esters, C20-D3-9-cis-retinol, C20-D3-9-cis-retinaldehye, C20-D3-9-cis-retinyl esters, C20-D3-11-cis-retinol, C20-D3-11-cis-retinaldehye, C20-D3-11-cis-retinyl esters, and C20-D3-C20′-D3-β-carotene.

In some embodiments of the method, the C20-D3-visual-chromophore-producing compound is in a form packaged in chylomicrons.

In some embodiments of the method, the selective RBP4 antagonist is a compound having the structure:

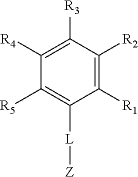

wherein L is a linking group having the structure

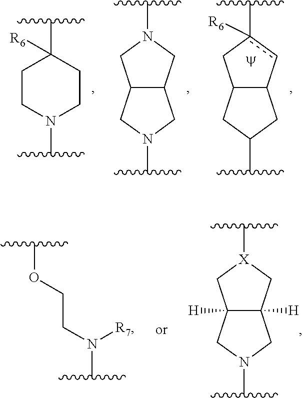

and
Z is a group having the structure

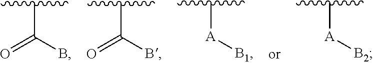

wherein
R₁, R₂, R₃, R₄, and R₅ are each independently H, halogen, CF₃, C₁-C₄ alkyl, aryl, or heteroaryl;
R₆ is H, OH, or halogen, or is absent;
| is absent or present, and when present, is a bond;
B is a substituted or unsubstituted heterobicycle, pyridazine, pyrazole, pyrazine, thiadiazole, or triazole, wherein
the heterobicycle is other than chloro-substituted indole; and
the pyrazole, when substituted, is substituted with other than trifluoromethyl;
   B′ is a substituted or unsubstituted phenyl, pyridine, pyrimidine, benzyl, pyrrolidine, sulfolane, oxetane, CO₂H, or (C₁-C₄ alkylenyl)-CO₂H, wherein
the substituted phenyl is substituted with other than trifluoromethyl or 3-(methyl carboxylate),
the substituted pyridine is substituted with other than trifluoromethyl,
the substituted pyrrolidine is substituted with other than hydroxamic acid, and
the substituted or unsubstituted pyrrolidine is bound to the carbonyl through a carbon-carbon bond;
   A is absent or present, and when present, is

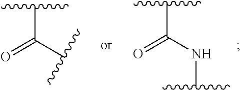

B₁ is a substituted or unsubstituted monocycle, bicycle, heteromonocycle, heterobicycle, benzyl, CO₂H, or (C₁-C₄ alkylenyl)-CO₂H,
wherein when B₁ is CO₂H, then A is present and is

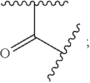 [SMILES: *C(*)=O]

R₇ is alkyl;

### X is N or CR_{B},

wherein R₈ is H, OH, or halogen; and
   B₂ has the structure:

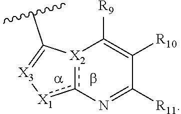

wherein
α and β are each a bond that is present or absent;
X₁ is N, NH, or NR₉₉,
wherein R₉₉ is alkyl, alkenyl, or alkynyl;

### X₂ is C or N;

### X₃ is CH or N;

R₉, R₁₀, and R₁₁ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃, wherein
X₁, X₂, and X₃ are each N, α is present, and β is absent; or
X₁ is NH, X₂ is C, X₃ is CH, α is absent, and β is present; or
X₁ is N, X₂ is N, X₃ is CH, α is present, and β is absent; or
X₁ is NH or NR₉₉, X₂ is C, X₃ is N, α is absent, and β is present, wherein when X₁ is NH, X₂ is C, X₃ is N, α is absent, and β is present, then one of R₉, R₁₀, and R₁₁ is other than H;
   or
   B₂ has the structure:

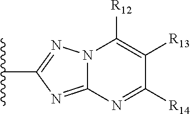

wherein
R₁₂, R₁₃, and R₁₄ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
or a pharmaceutically acceptable salt thereof.

In some embodiments of the method, the selective RPB4 antagonist is the compound wherein R₁, R₂, R₃, R₄, and R₅ are each independently H, halogen, CF₃, or C₁-C₄ alkyl.

In some embodiments of the method, the selective RPB4 antagonist is R₆ the compound wherein L is

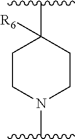

### and Z is

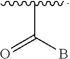

In some embodiments of the method, the selective RBP4 antagonist is the compound wherein L is

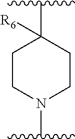

### and Z is 0

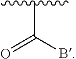

In some embodiments of the method, the selective RBP4 antagonist is the compound wherein
L is

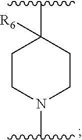

Z is

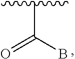

two or more of R₁, R₂, R₃, R₄, and R₅ are other than H, and when
   R₁ is CF₃, R₂ is H, R₃ is F, R₄ is H, and R₅ is H, or
   R₁ is H, R₂ is CF₃, R₃ is H, R₄ is CF₃, and R₅ is H, or
   R₁ is Cl, R₂ is H, R₃ is H, R₄ is F, and R₅ is H, or
   R₁ is CF₃, R₂ is H, R₃ is F, R₄ is H, and R₅ is H, or
   R₁ is CF₃, R₂ is F, R₃ is H, R₄ is H, and R₅ is H, or
   R₁ is Cl, R₂ is F, R₃ is H, R₄ is H, and R₅ is H,
then B is other than

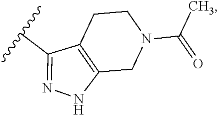 [SMILES: *c1n[nH]c2c1CCN(C(C)=O)C2]

In some embodiments of the method, the selective RBP4 antagonist is the compound wherein L is

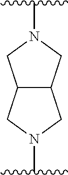 [SMILES: *N1CC2CN(*)CC2C1]

### and Z is

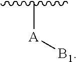

In some embodiments of the method, the selective RBP4 antagonist is the compound wherein
L is

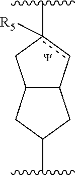

Z is

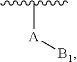

R₆ is absent or present, and when present, is H, OH, or halogen, and
when ψ is present, then R₆ is absent, and when [symbol] is absent, then R₆ is present.

In some embodiments of the method, the selective RBP4 antagonist is the compound wherein L is

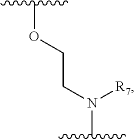

### and Z is

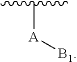

In some embodiments of the method, the selective RBP4 antagonist is the compound wherein L is

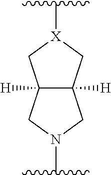

### and Z is B₂.

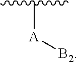

In some embodiments of the method, the selective RBP4 antagonist is the compound wherein
L is

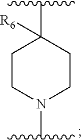

Z is

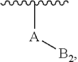

R₆ is H, and
A is

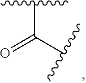 [SMILES: *C(*)=O]

In some embodiments of the method, the selective RBP4 antagonist is the compound wherein L is

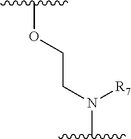

### and Z is

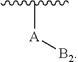

In some embodiments of the method, the selective RBP4 antagonist is

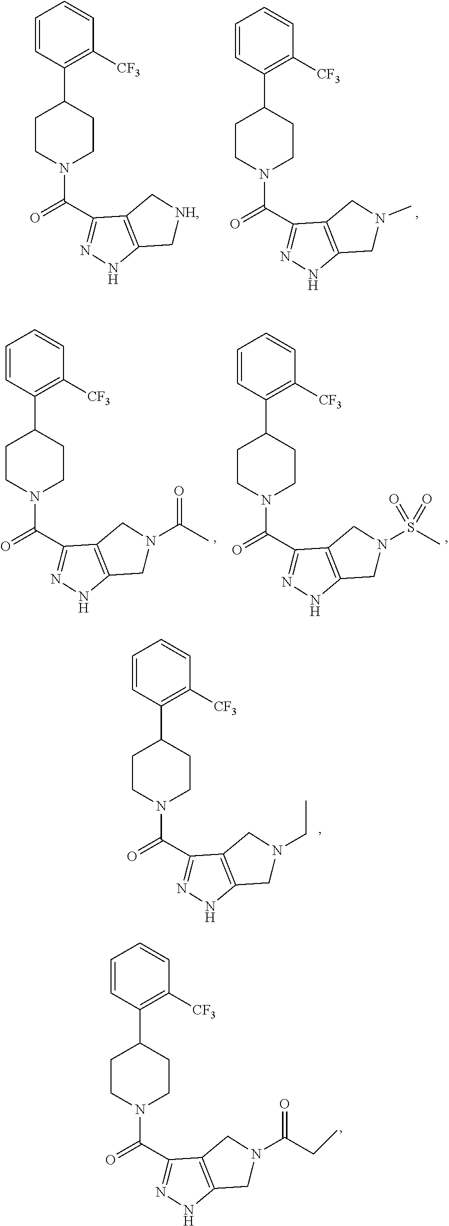

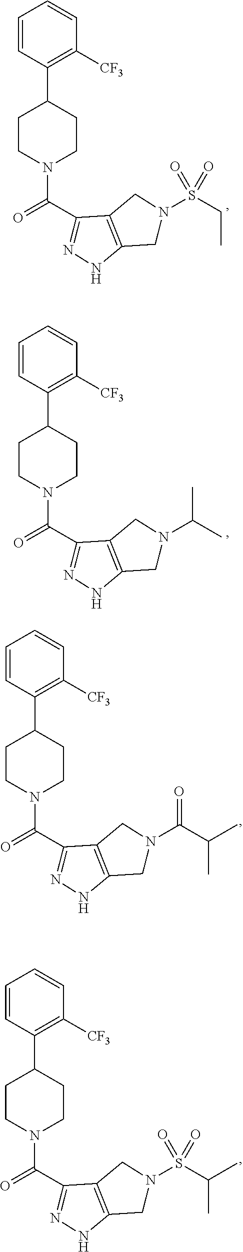 [SMILES: CCS(=O)(=O)N1Cc2[nH]nc(C(=O)N3CCC(c4ccccc4C(F)(F)F)CC3)c2C1 ; CC(C)N1Cc2[nH]nc(C(=O)N3CCC(c4ccccc4C(F)(F)F)CC3)c2C1 ; CC(C)C(=O)N1Cc2[nH]nc(C(=O)N3CCC(c4ccccc4C(F)(F)F)CC3)c2C1 ; CC(C)S(=O)(=O)N1Cc2[nH]nc(C(=O)N3CCC(c4ccccc4C(F)(F)F)CC3)c2C1]

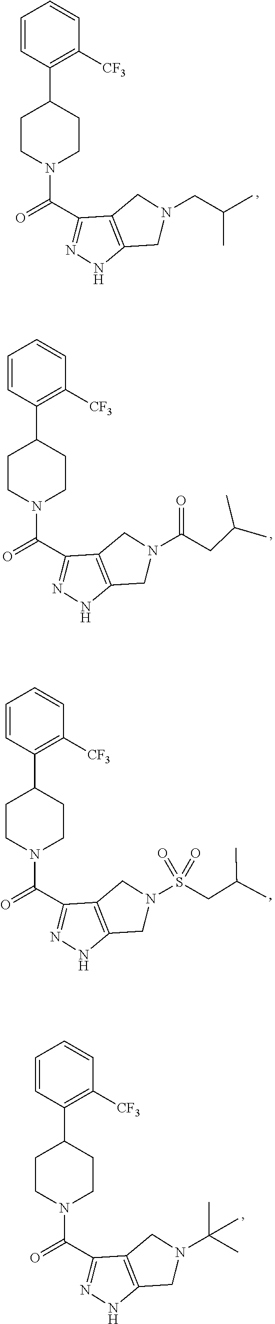 [SMILES: CC(C)CN1Cc2[nH]nc(C(=O)N3CCC(c4ccccc4C(F)(F)F)CC3)c2C1 ; CC(C)CC(=O)N1Cc2[nH]nc(C(=O)N3CCC(c4ccccc4C(F)(F)F)CC3)c2C1 ; CC(C)CS(=O)(=O)N1Cc2[nH]nc(C(=O)N3CCC(c4ccccc4C(F)(F)F)CC3)c2C1 ; CC(C)(C)N1Cc2[nH]nc(C(=O)N3CCC(c4ccccc4C(F)(F)F)CC3)c2C1]

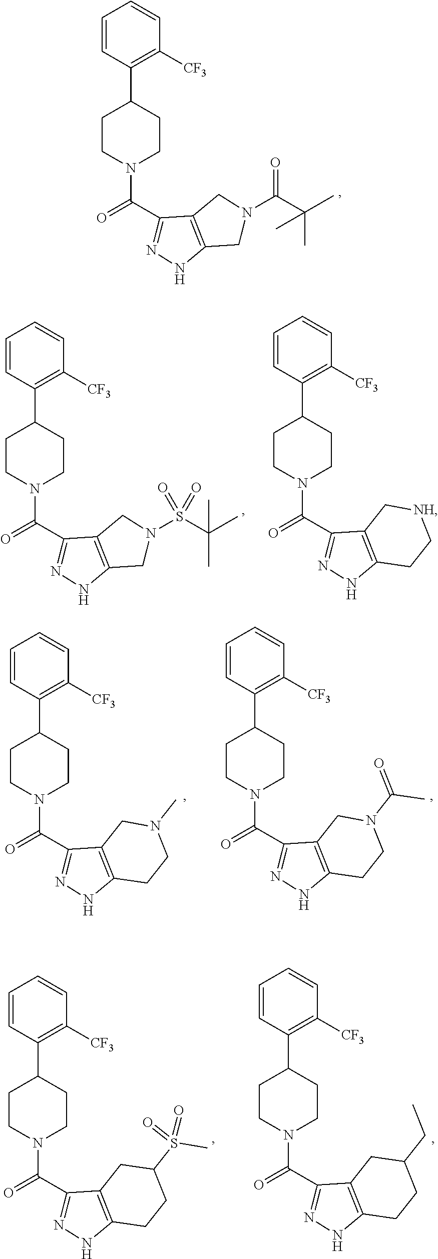

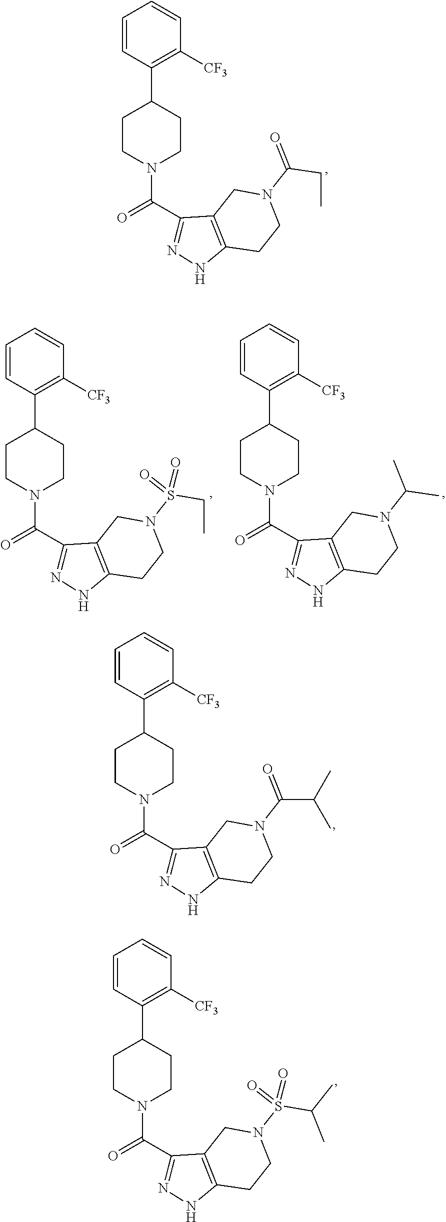

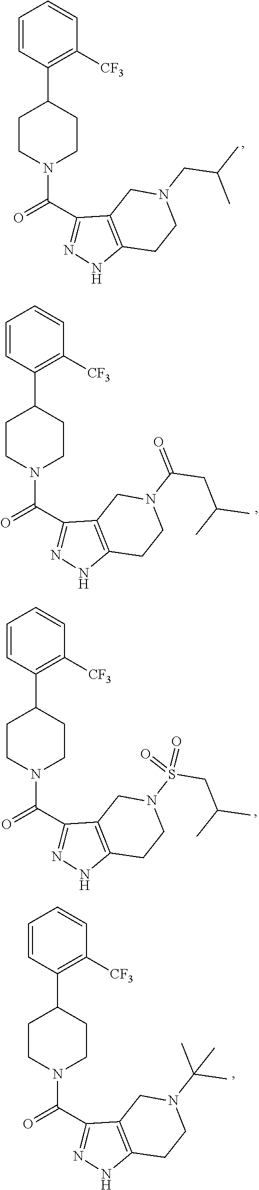

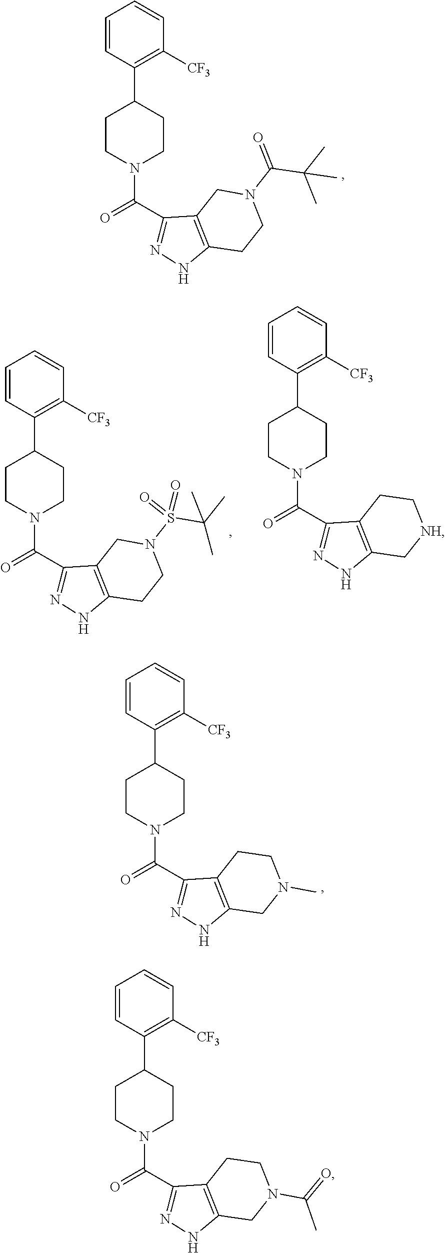 [SMILES: CC(C)(C)C(=O)N1CCc2[nH]nc(C(=O)N3CCC(c4ccccc4C(F)(F)F)CC3)c2C1 ; CC(C)(C)S(=O)(=O)N1CCc2[nH]nc(C(=O)N3CCC(c4ccccc4C(F)(F)F)CC3)c2C1 ; O=C(c1n[nH]c2c1CCNC2)N1CCC(c2ccccc2C(F)(F)F)CC1 ; CN1CCc2c(C(=O)N3CCC(c4ccccc4C(F)(F)F)CC3)n[nH]c2C1 ; CC(=O)N1CCc2c(C(=O)N3CCC(c4ccccc4C(F)(F)F)CC3)n[nH]c2C1]

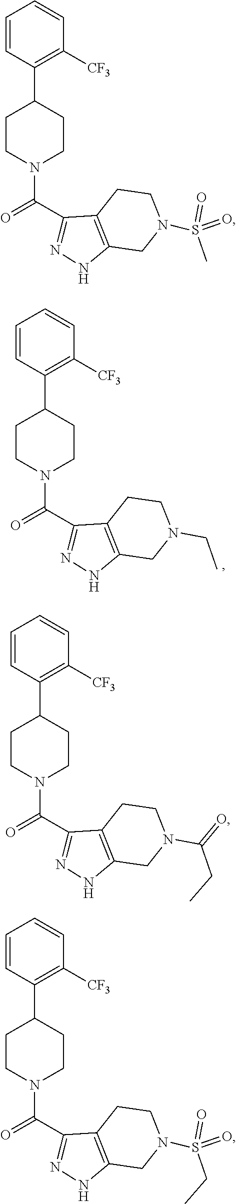 [SMILES: CS(=O)(=O)N1CCc2c(C(=O)N3CCC(c4ccccc4C(F)(F)F)CC3)n[nH]c2C1 ; CCN1CCc2c(C(=O)N3CCC(c4ccccc4C(F)(F)F)CC3)n[nH]c2C1 ; CCC(=O)N1CCc2c(C(=O)N3CCC(c4ccccc4C(F)(F)F)CC3)n[nH]c2C1 ; CCS(=O)(=O)N1CCc2c(C(=O)N3CCC(c4ccccc4C(F)(F)F)CC3)n[nH]c2C1]

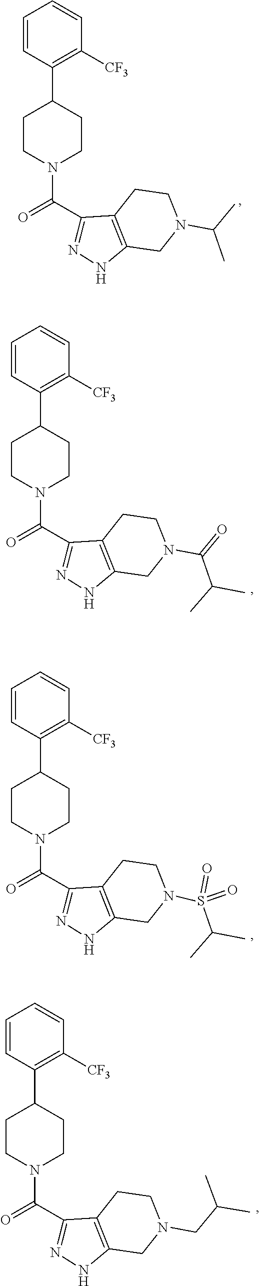 [SMILES: CC(C)N1CCc2c(C(=O)N3CCC(c4ccccc4C(F)(F)F)CC3)n[nH]c2C1 ; CC(C)C(=O)N1CCc2c(C(=O)N3CCC(c4ccccc4C(F)(F)F)CC3)n[nH]c2C1 ; CC(C)S(=O)(=O)N1CCc2c(C(=O)N3CCC(c4ccccc4C(F)(F)F)CC3)n[nH]c2C1 ; CC(C)CN1CCc2c(C(=O)N3CCC(c4ccccc4C(F)(F)F)CC3)n[nH]c2C1]

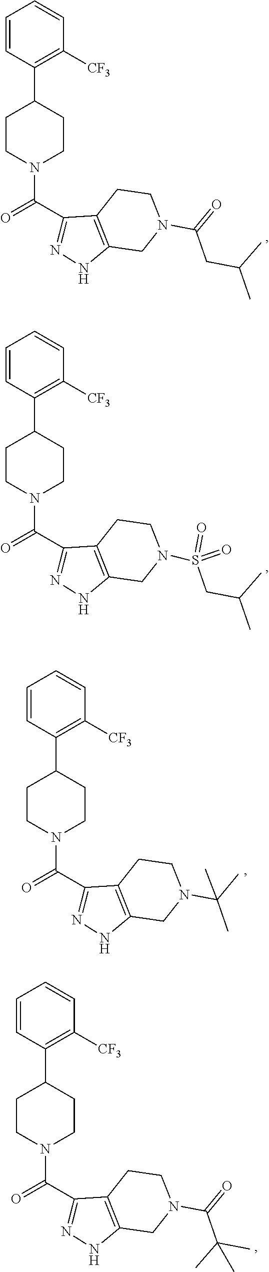 [SMILES: CC(C)CC(=O)N1CCc2c(C(=O)N3CCC(c4ccccc4C(F)(F)F)CC3)n[nH]c2C1 ; CC(C)CS(=O)(=O)N1CCc2c(C(=O)N3CCC(c4ccccc4C(F)(F)F)CC3)n[nH]c2C1 ; CC(C)(C)N1CCc2c(C(=O)N3CCC(c4ccccc4C(F)(F)F)CC3)n[nH]c2C1 ; CC(C)(C)C(=O)N1CCc2c(C(=O)N3CCC(c4ccccc4C(F)(F)F)CC3)n[nH]c2C1]

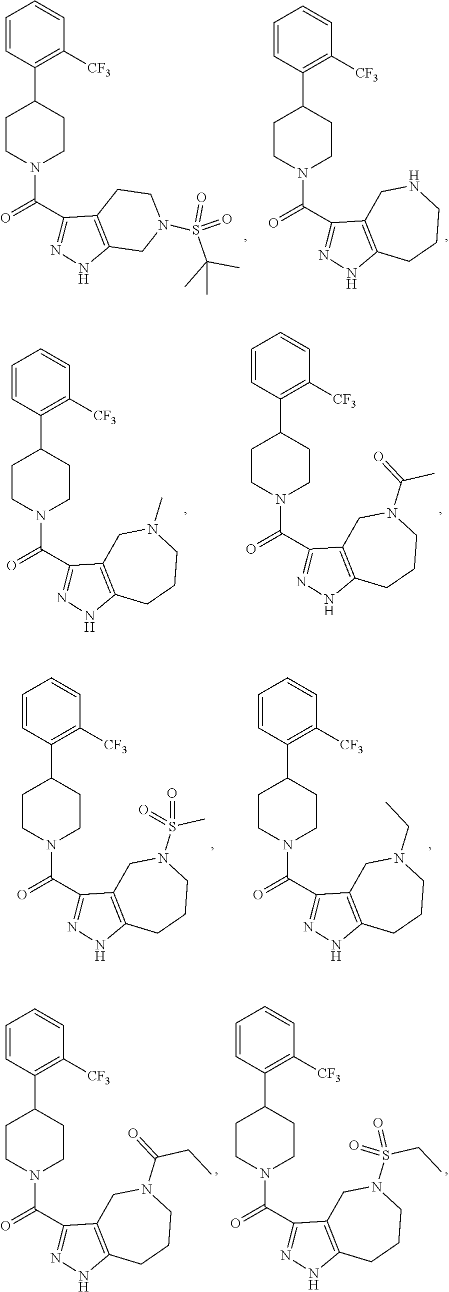

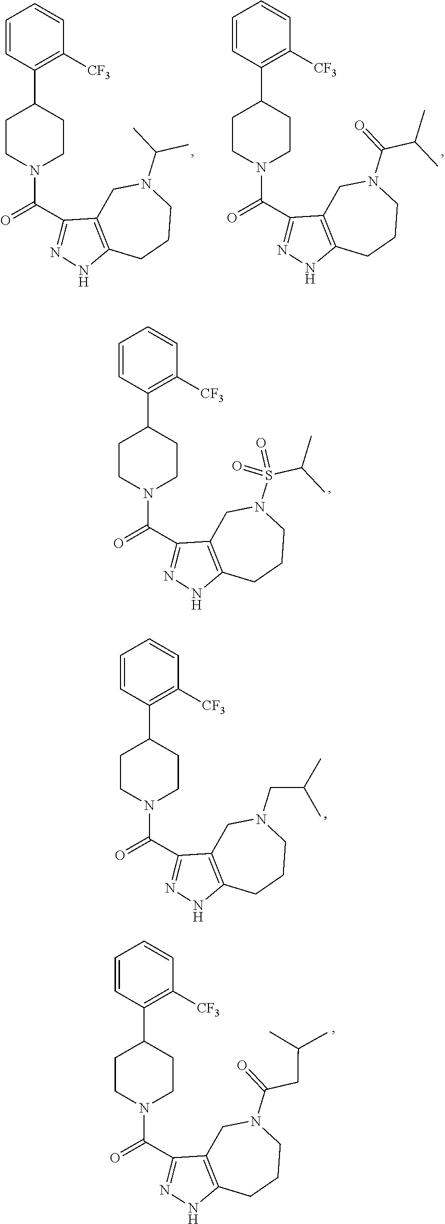

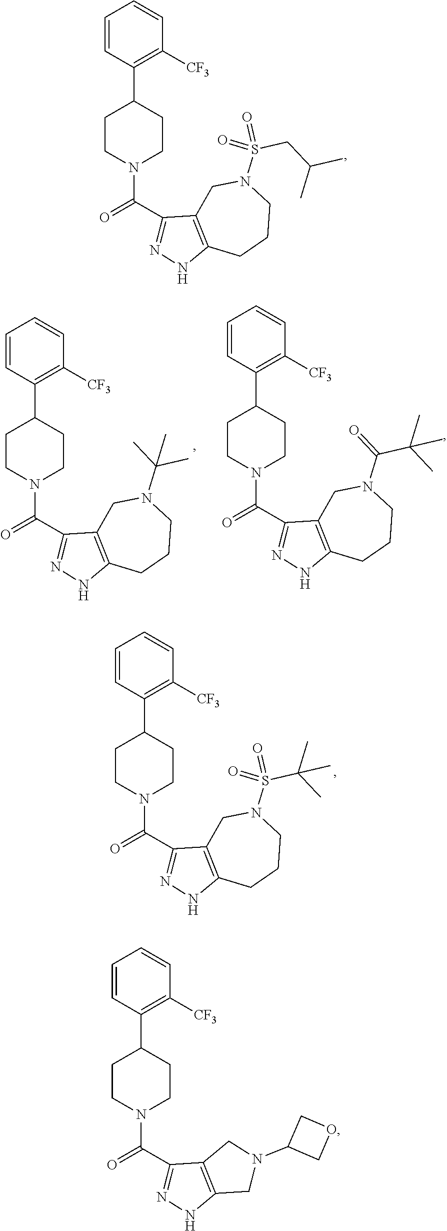

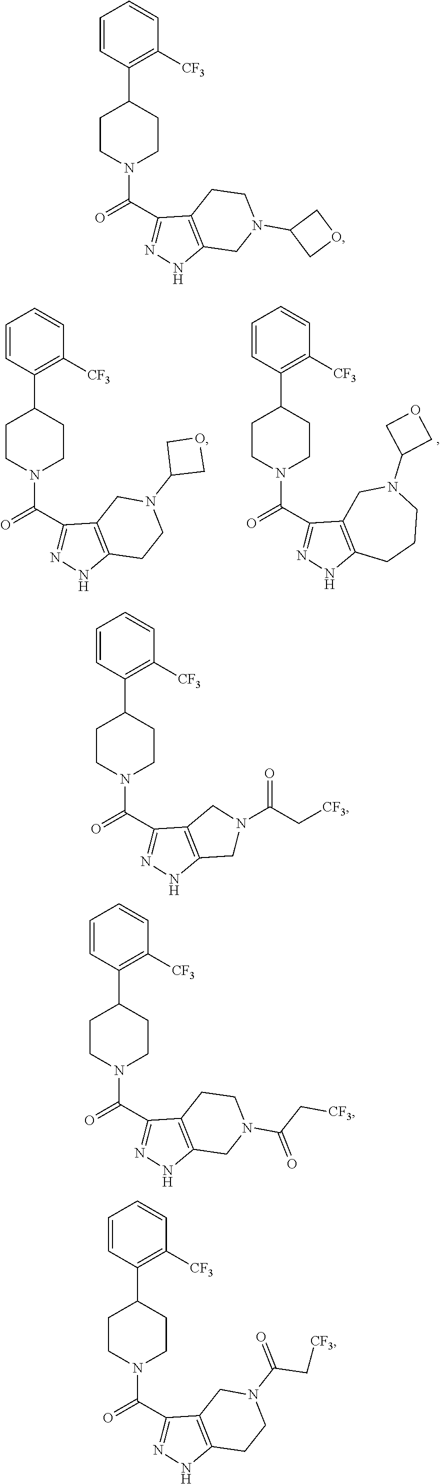

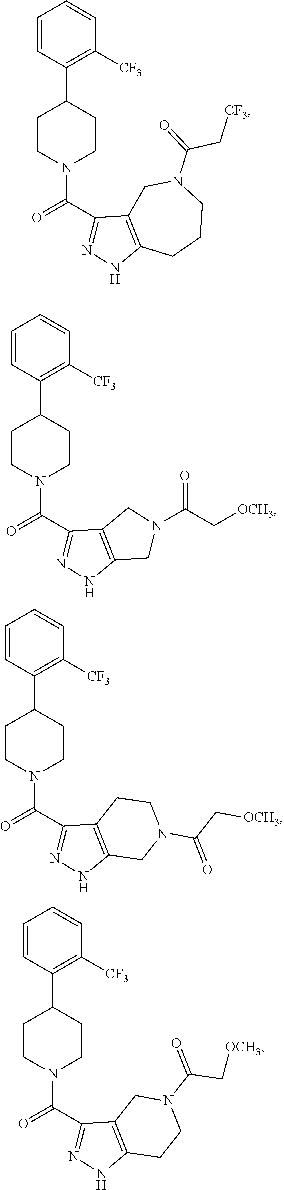

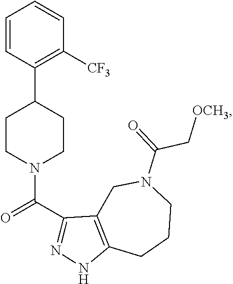 [SMILES: COCC(=O)N1CCCc2[nH]nc(C(=O)N3CCC(c4ccccc4C(F)(F)F)CC3)c2C1]

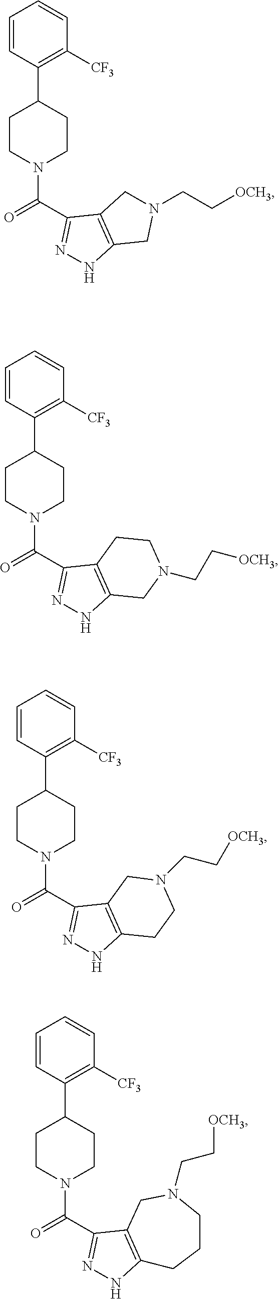 [SMILES: COCCN1Cc2[nH]nc(C(=O)N3CCC(c4ccccc4C(F)(F)F)CC3)c2C1 ; COCCN1CCc2c(C(=O)N3CCC(c4ccccc4C(F)(F)F)CC3)n[nH]c2C1 ; COCCN1CCc2[nH]nc(C(=O)N3CCC(c4ccccc4C(F)(F)F)CC3)c2C1 ; COCCN1CCCc2[nH]nc(C(=O)N3CCC(c4ccccc4C(F)(F)F)CC3)c2C1]

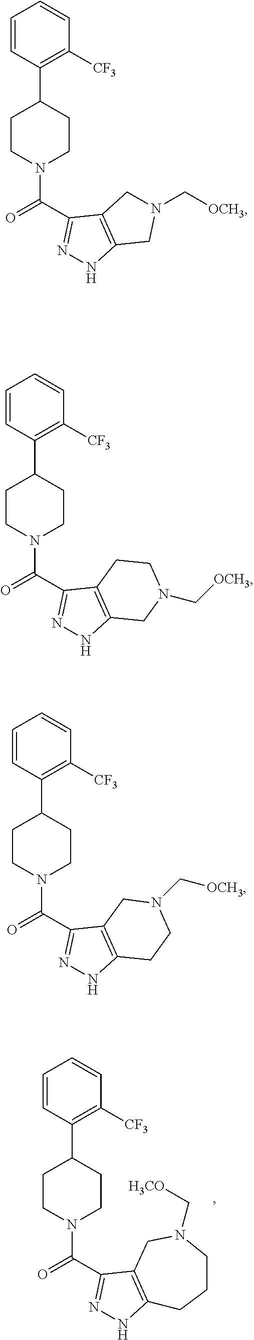 [SMILES: COCN1Cc2[nH]nc(C(=O)N3CCC(c4ccccc4C(F)(F)F)CC3)c2C1 ; COCN1CCc2c(C(=O)N3CCC(c4ccccc4C(F)(F)F)CC3)n[nH]c2C1 ; COCN1CCc2[nH]nc(C(=O)N3CCC(c4ccccc4C(F)(F)F)CC3)c2C1 ; COCN1CCCc2[nH]nc(C(=O)N3CCC(c4ccccc4C(F)(F)F)CC3)c2C1]

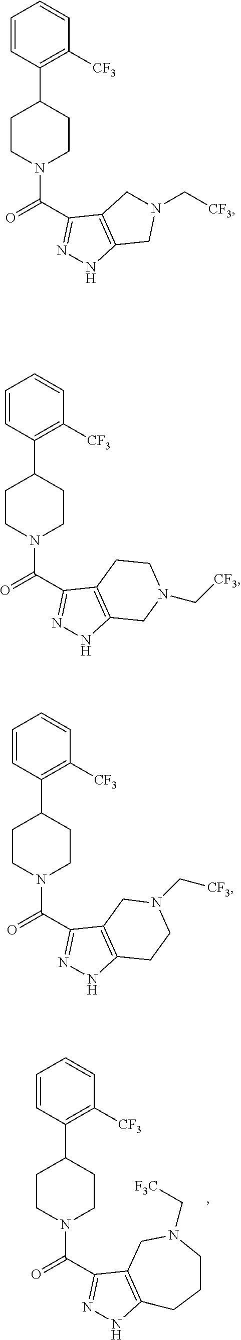 [SMILES: O=C(c1n[nH]c2c1CN(CC(F)(F)F)C2)N1CCC(c2ccccc2C(F)(F)F)CC1 ; O=C(c1n[nH]c2c1CCN(CC(F)(F)F)C2)N1CCC(c2ccccc2C(F)(F)F)CC1 ; O=C(c1n[nH]c2c1CN(CC(F)(F)F)CC2)N1CCC(c2ccccc2C(F)(F)F)CC1 ; O=C(c1n[nH]c2c1CN(CC(F)(F)F)CCC2)N1CCC(c2ccccc2C(F)(F)F)CC1]

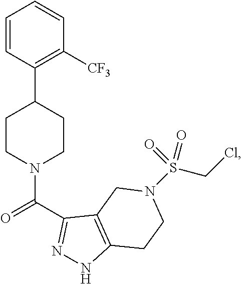 [SMILES: O=C(c1n[nH]c2c1CN(S(=O)(=O)CCl)CC2)N1CCC(c2ccccc2C(F)(F)F)CC1]

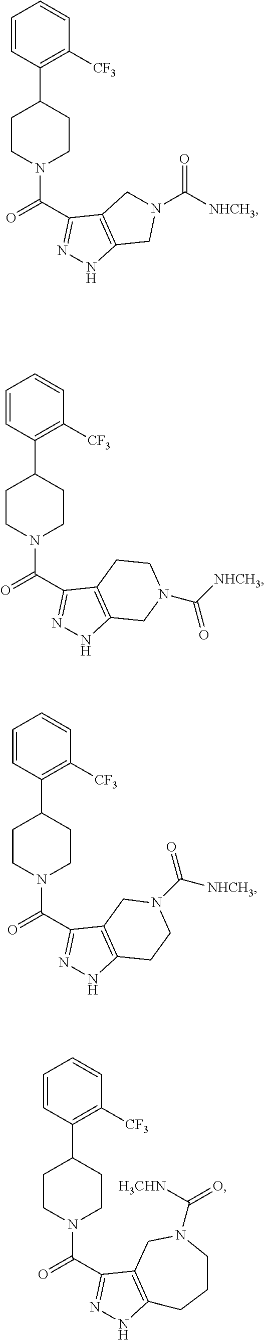

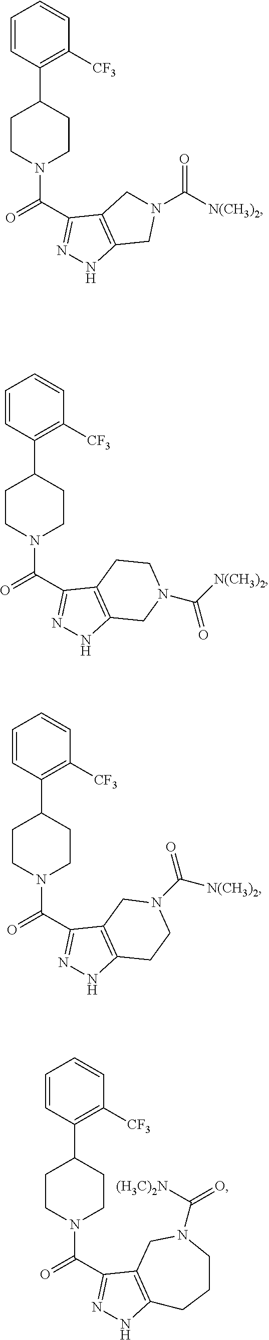

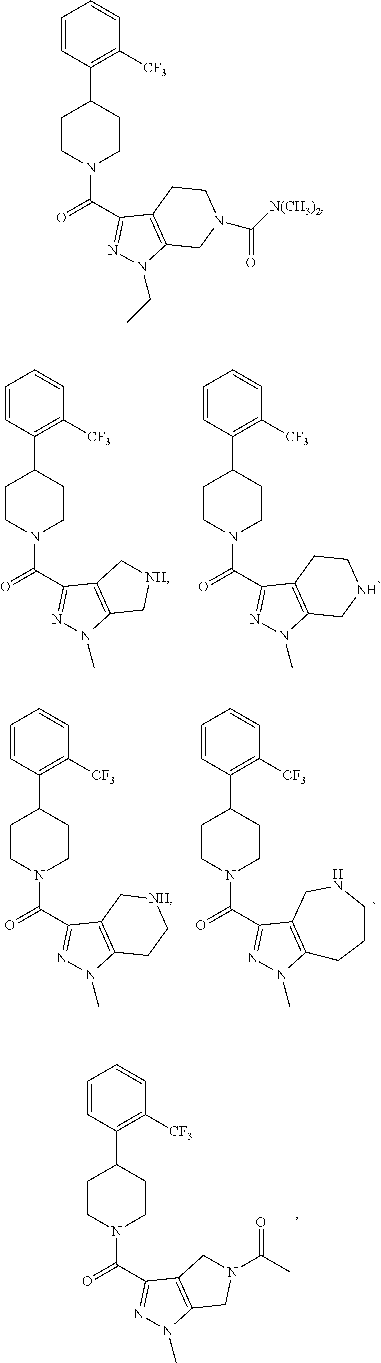

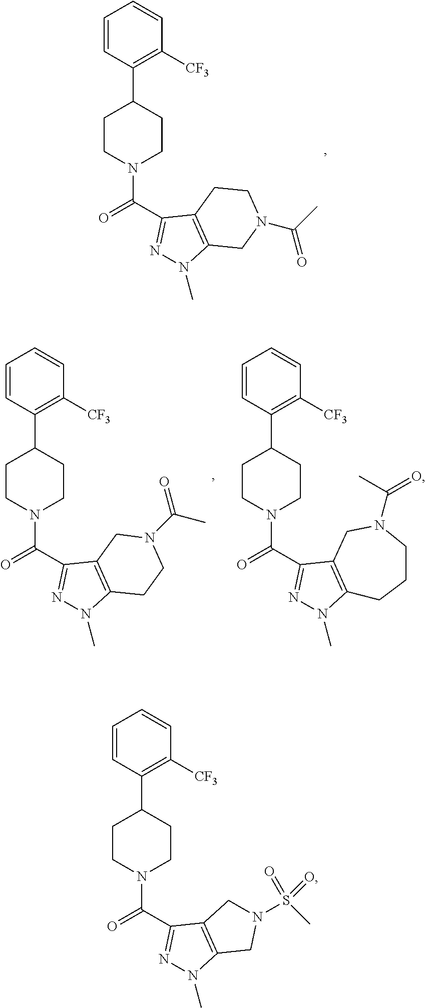

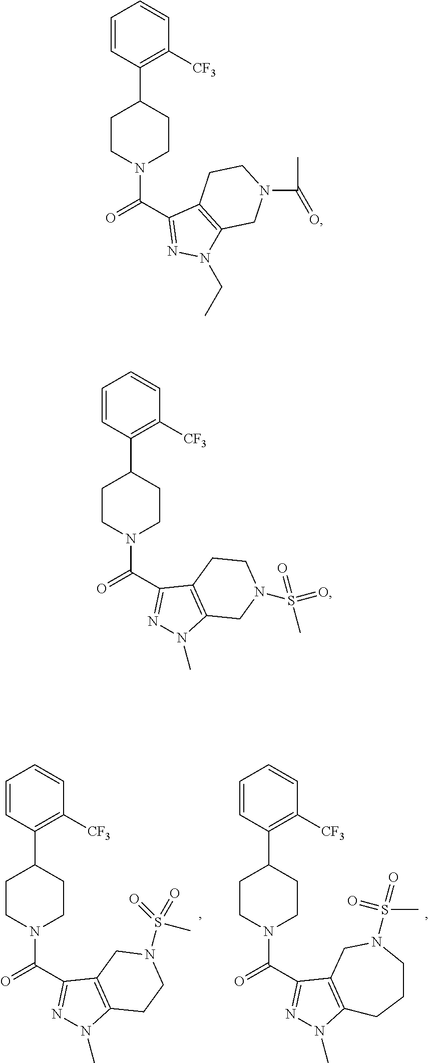 [SMILES: CCn1nc(C(=O)N2CCC(c3ccccc3C(F)(F)F)CC2)c2c1CN(C(C)=O)CC2 ; Cn1nc(C(=O)N2CCC(c3ccccc3C(F)(F)F)CC2)c2c1CN(S(C)(=O)=O)CC2 ; Cn1nc(C(=O)N2CCC(c3ccccc3C(F)(F)F)CC2)c2c1CCN(S(C)(=O)=O)C2 ; Cn1nc(C(=O)N2CCC(c3ccccc3C(F)(F)F)CC2)c2c1CCCN(S(C)(=O)=O)C2]

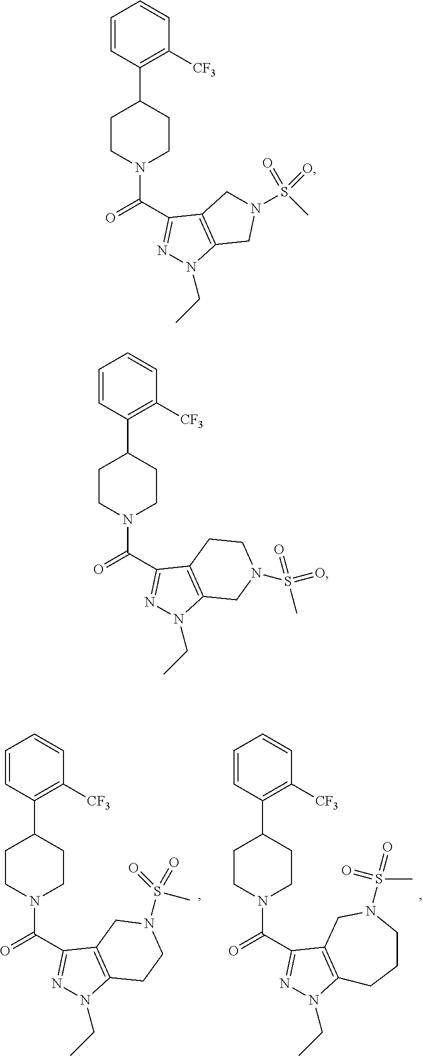 [SMILES: CCn1nc(C(=O)N2CCC(c3ccccc3C(F)(F)F)CC2)c2c1CN(S(C)(=O)=O)C2 ; CCn1nc(C(=O)N2CCC(c3ccccc3C(F)(F)F)CC2)c2c1CN(S(C)(=O)=O)CC2 ; CCn1nc(C(=O)N2CCC(c3ccccc3C(F)(F)F)CC2)c2c1CCN(S(C)(=O)=O)C2 ; CCn1nc(C(=O)N2CCC(c3ccccc3C(F)(F)F)CC2)c2c1CCCN(S(C)(=O)=O)C2]

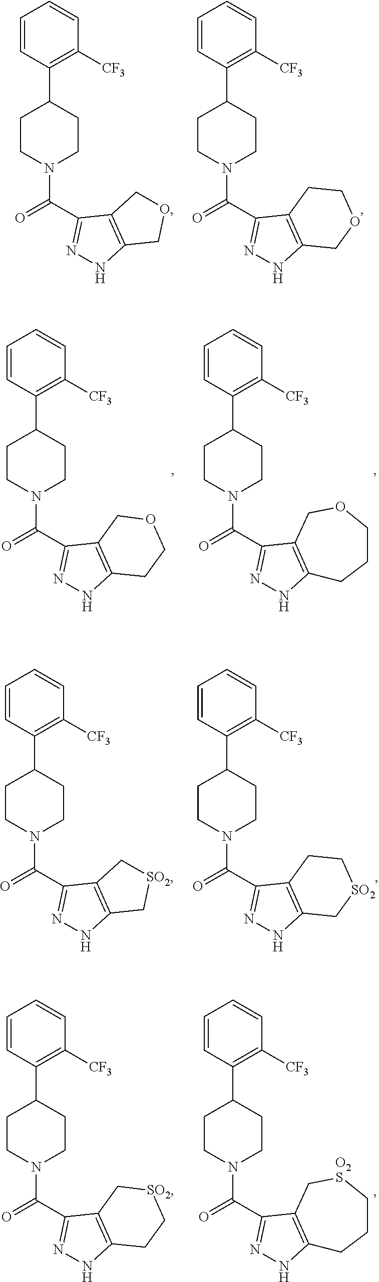

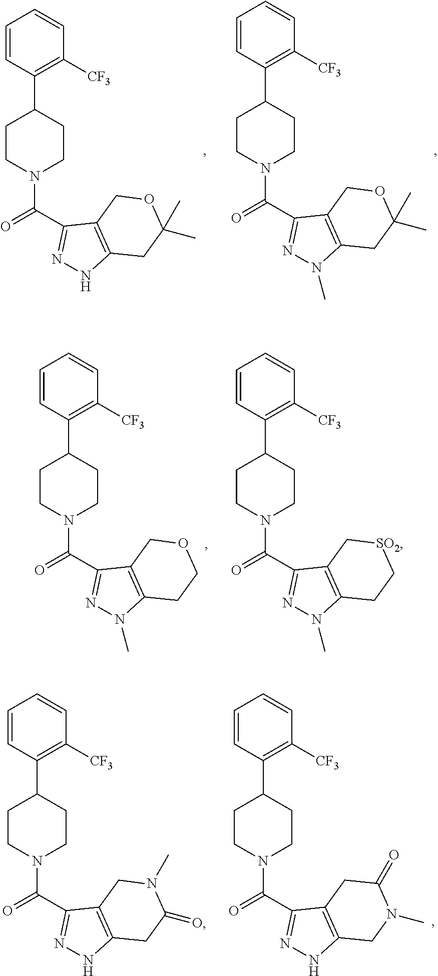

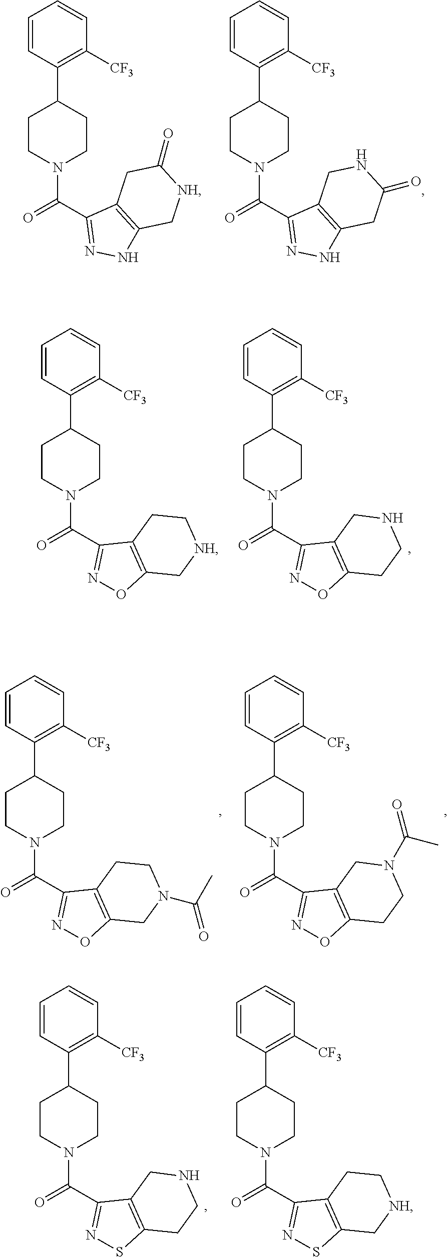

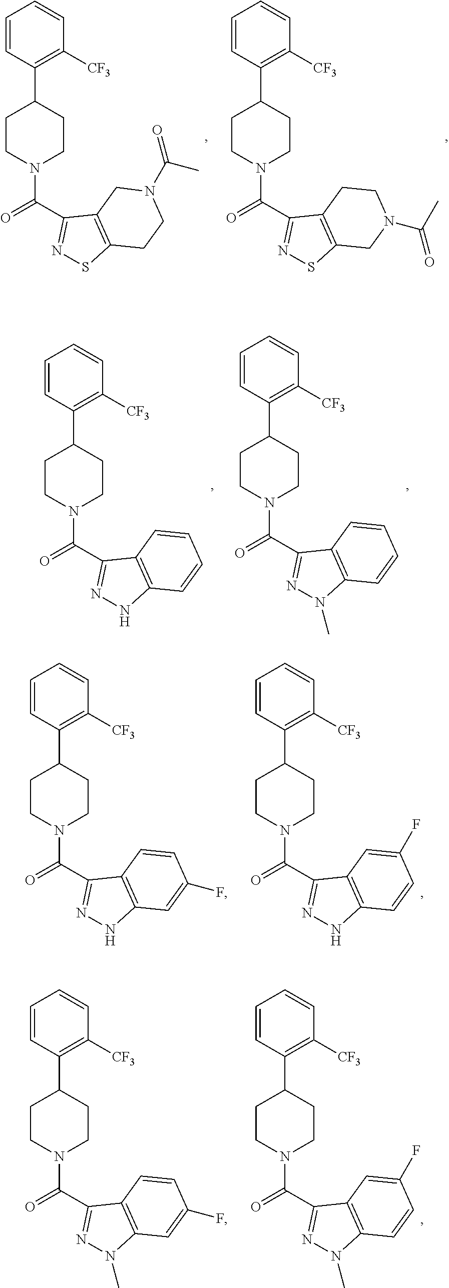

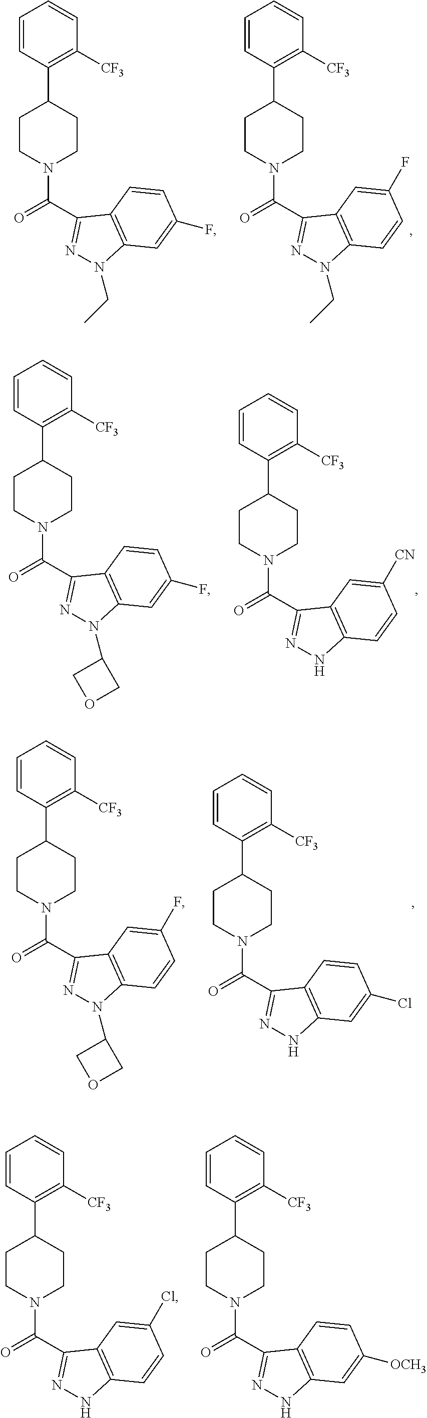

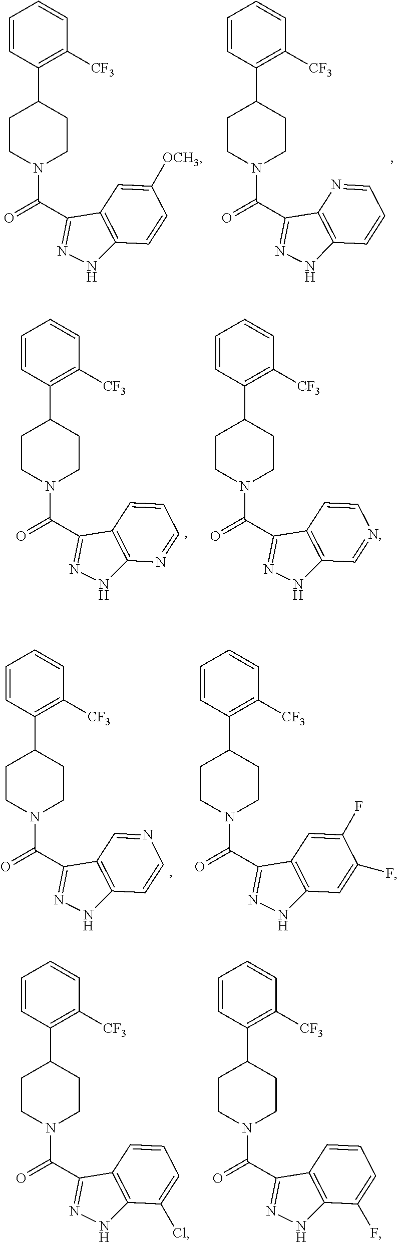

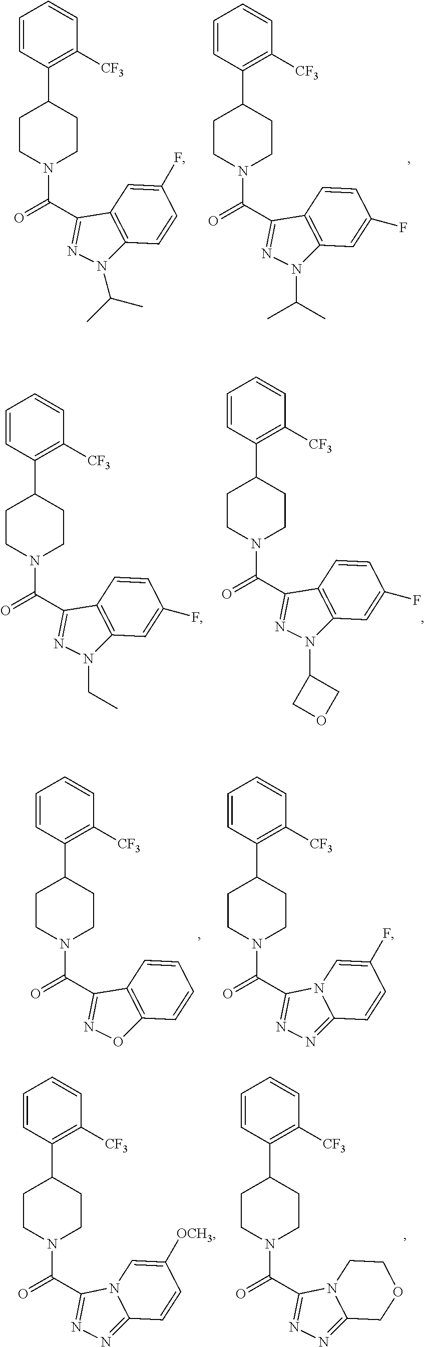

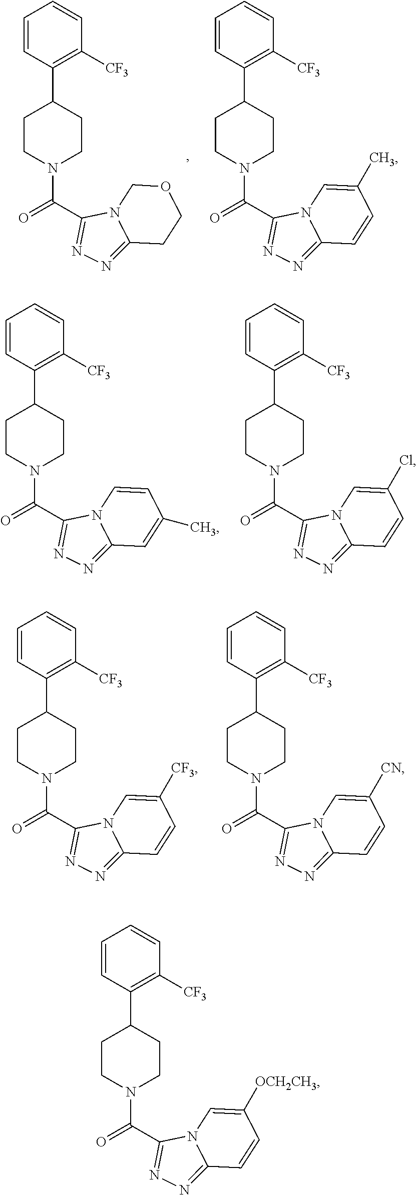

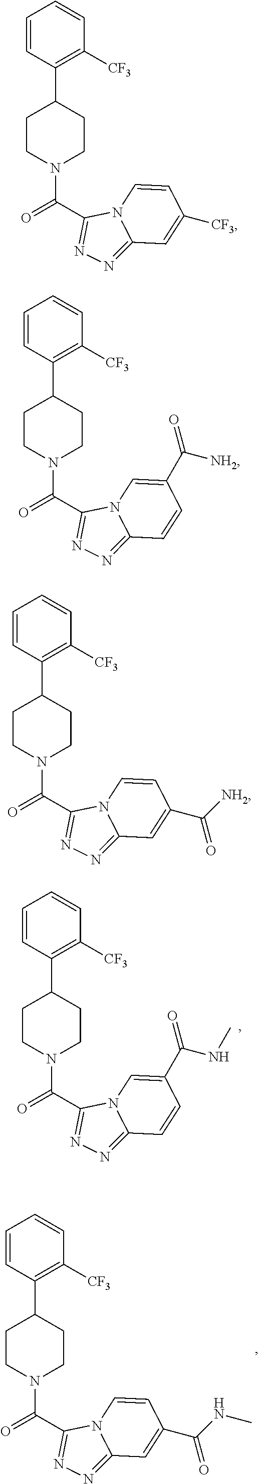 [SMILES: O=C(c1nnc2cc(C(F)(F)F)ccn12)N1CCC(c2ccccc2C(F)(F)F)CC1 ; NC(=O)c1ccc2nnc(C(=O)N3CCC(c4ccccc4C(F)(F)F)CC3)n2c1 ; NC(=O)c1ccn2c(C(=O)N3CCC(c4ccccc4C(F)(F)F)CC3)nnc2c1 ; CNC(=O)c1ccc2nnc(C(=O)N3CCC(c4ccccc4C(F)(F)F)CC3)n2c1 ; CNC(=O)c1ccn2c(C(=O)N3CCC(c4ccccc4C(F)(F)F)CC3)nnc2c1]

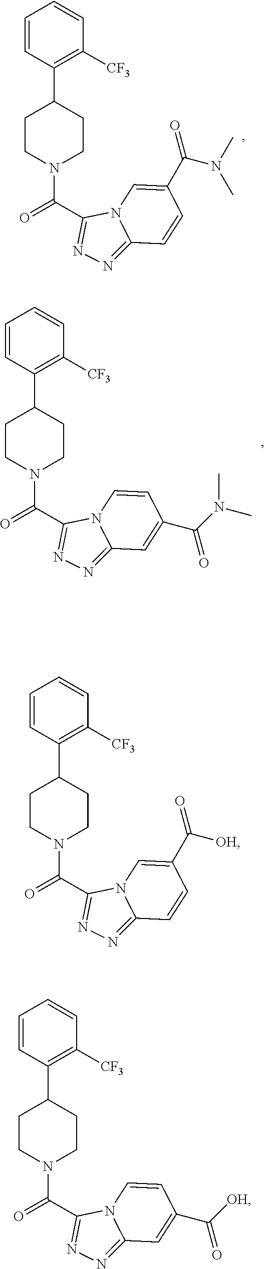 [SMILES: CN(C)C(=O)c1ccc2nnc(C(=O)N3CCC(c4ccccc4C(F)(F)F)CC3)n2c1 ; CN(C)C(=O)c1ccn2c(C(=O)N3CCC(c4ccccc4C(F)(F)F)CC3)nnc2c1 ; O=C(O)c1ccc2nnc(C(=O)N3CCC(c4ccccc4C(F)(F)F)CC3)n2c1 ; O=C(O)c1ccn2c(C(=O)N3CCC(c4ccccc4C(F)(F)F)CC3)nnc2c1]

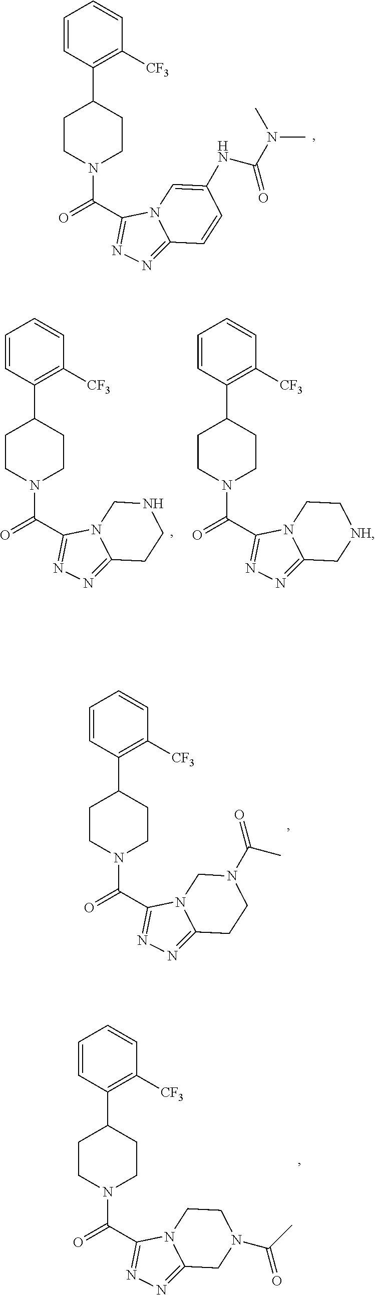

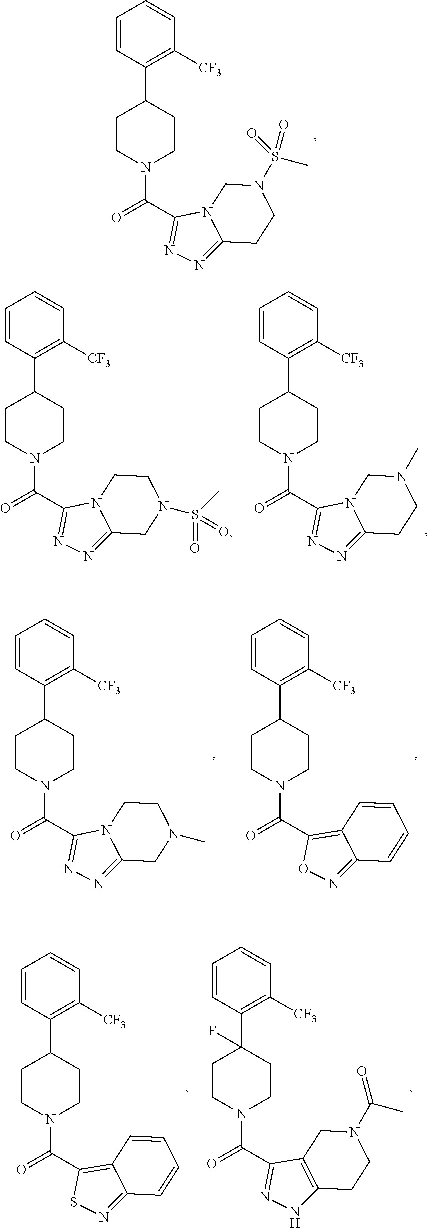

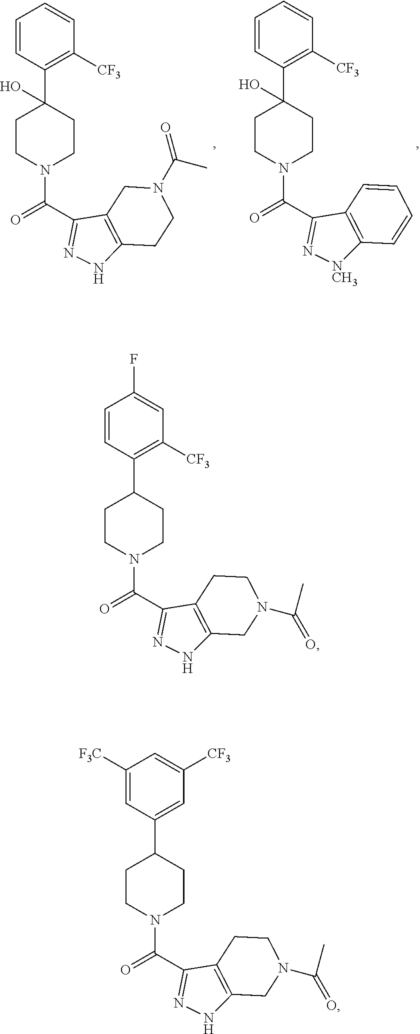 [SMILES: CC(=O)N1CCc2[nH]nc(C(=O)N3CCC(O)(c4ccccc4C(F)(F)F)CC3)c2C1 ; Cn1nc(C(=O)N2CCC(O)(c3ccccc3C(F)(F)F)CC2)c2ccccc21 ; CC(=O)N1CCc2c(C(=O)N3CCC(c4ccc(F)cc4C(F)(F)F)CC3)n[nH]c2C1 ; CC(=O)N1CCc2c(C(=O)N3CCC(c4cc(C(F)(F)F)cc(C(F)(F)F)c4)CC3)n[nH]c2C1]

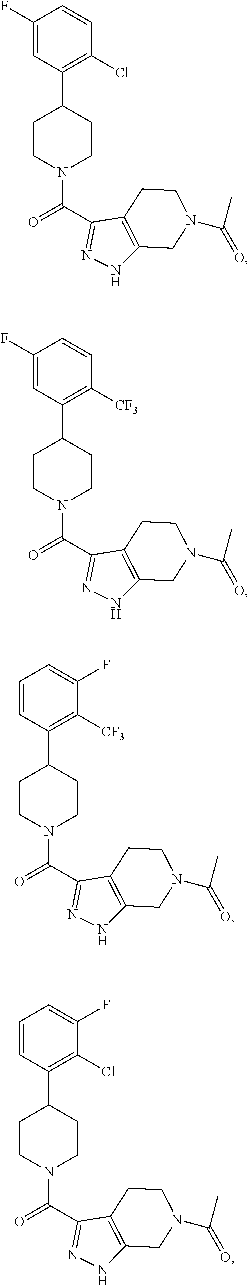 [SMILES: CC(=O)N1CCc2c(C(=O)N3CCC(c4cc(F)ccc4Cl)CC3)n[nH]c2C1 ; CC(=O)N1CCc2c(C(=O)N3CCC(c4cc(F)ccc4C(F)(F)F)CC3)n[nH]c2C1 ; CC(=O)N1CCc2c(C(=O)N3CCC(c4cccc(F)c4C(F)(F)F)CC3)n[nH]c2C1 ; CC(=O)N1CCc2c(C(=O)N3CCC(c4cccc(F)c4Cl)CC3)n[nH]c2C1]

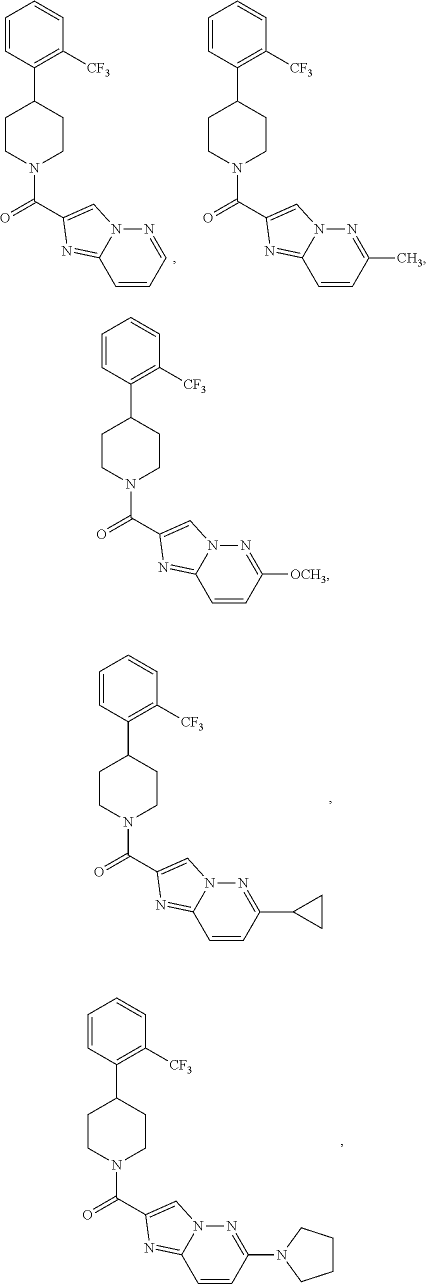

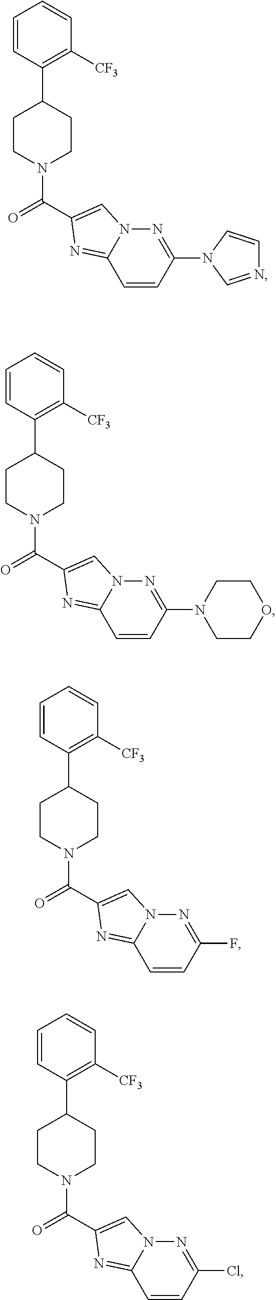 [SMILES: O=C(c1cn2nc(-n3ccnc3)ccc2n1)N1CCC(c2ccccc2C(F)(F)F)CC1 ; O=C(c1cn2nc(N3CCOCC3)ccc2n1)N1CCC(c2ccccc2C(F)(F)F)CC1 ; O=C(c1cn2nc(F)ccc2n1)N1CCC(c2ccccc2C(F)(F)F)CC1 ; O=C(c1cn2nc(Cl)ccc2n1)N1CCC(c2ccccc2C(F)(F)F)CC1]

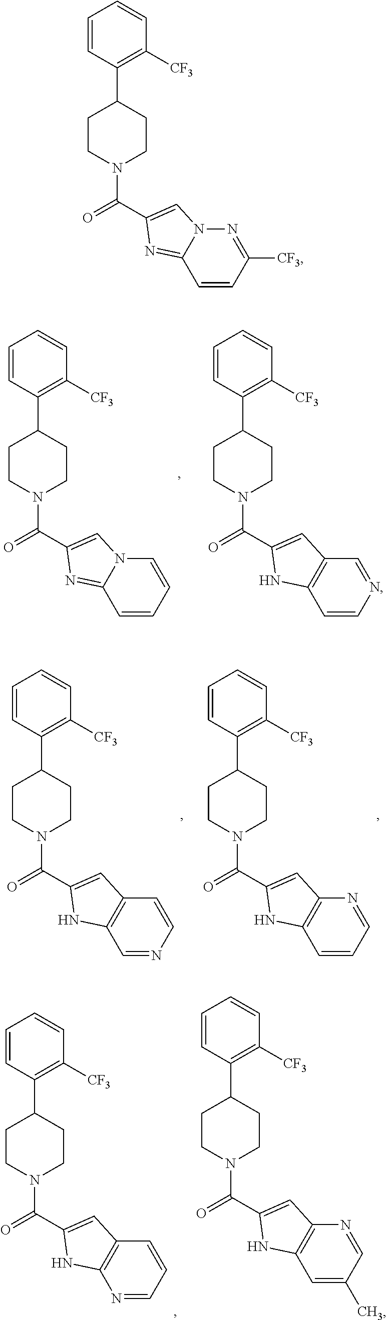

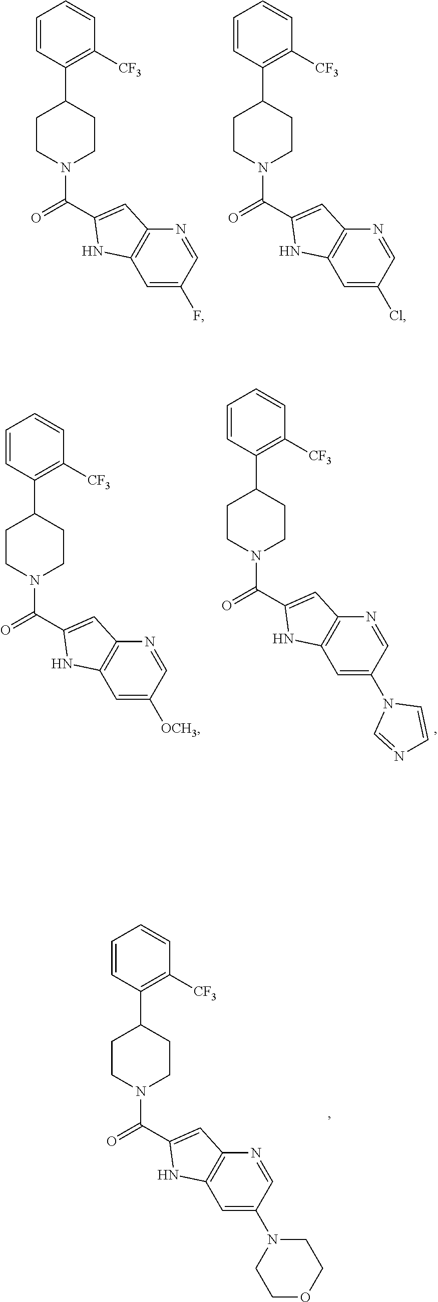 [SMILES: O=C(c1cc2ncc(F)cc2[nH]1)N1CCC(c2ccccc2C(F)(F)F)CC1 ; O=C(c1cc2ncc(Cl)cc2[nH]1)N1CCC(c2ccccc2C(F)(F)F)CC1 ; COc1cnc2cc(C(=O)N3CCC(c4ccccc4C(F)(F)F)CC3)[nH]c2c1 ; O=C(c1cc2ncc(-n3ccnc3)cc2[nH]1)N1CCC(c2ccccc2C(F)(F)F)CC1 ; O=C(c1cc2ncc(N3CCOCC3)cc2[nH]1)N1CCC(c2ccccc2C(F)(F)F)CC1]

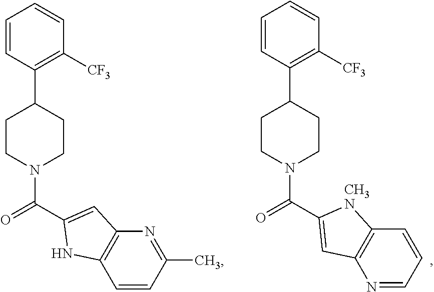

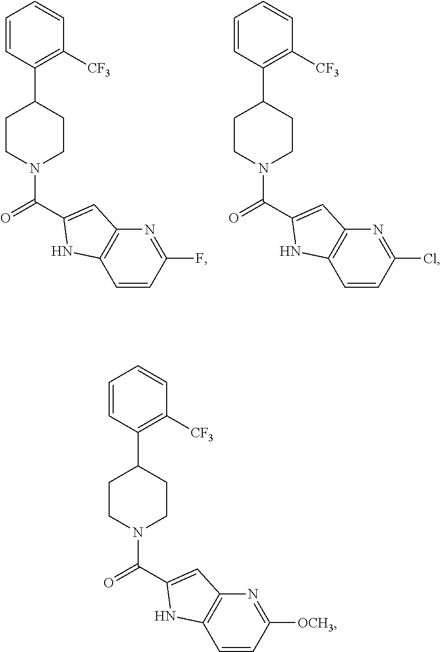 [SMILES: O=C(c1cc2nc(F)ccc2[nH]1)N1CCC(c2ccccc2C(F)(F)F)CC1 ; O=C(c1cc2nc(Cl)ccc2[nH]1)N1CCC(c2ccccc2C(F)(F)F)CC1 ; COc1ccc2[nH]c(C(=O)N3CCC(c4ccccc4C(F)(F)F)CC3)cc2n1]

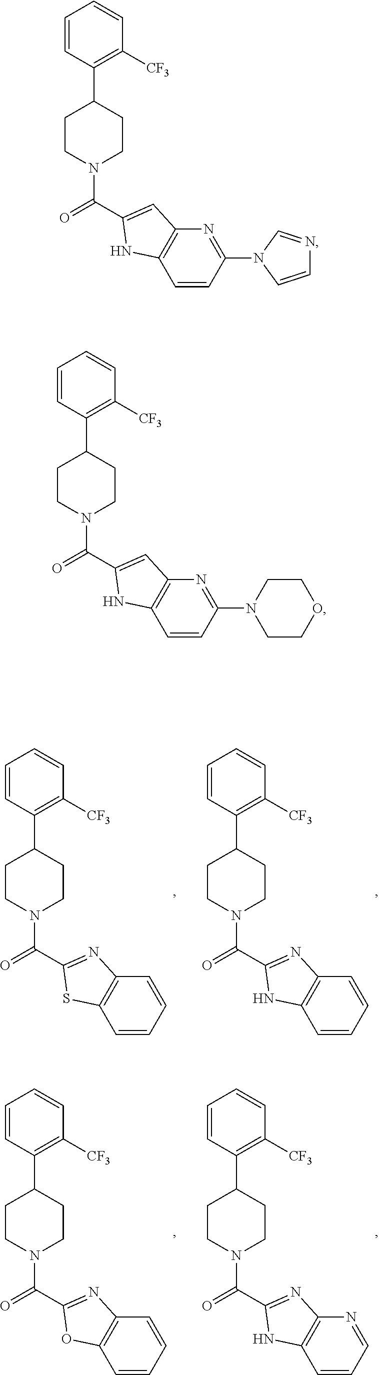 [SMILES: O=C(c1cc2nc(-n3ccnc3)ccc2[nH]1)N1CCC(c2ccccc2C(F)(F)F)CC1 ; O=C(c1cc2nc(N3CCOCC3)ccc2[nH]1)N1CCC(c2ccccc2C(F)(F)F)CC1 ; O=C(c1nc2ccccc2s1)N1CCC(c2ccccc2C(F)(F)F)CC1 ; O=C(c1nc2ccccc2[nH]1)N1CCC(c2ccccc2C(F)(F)F)CC1 ; O=C(c1nc2ccccc2o1)N1CCC(c2ccccc2C(F)(F)F)CC1 ; O=C(c1nc2ncccc2[nH]1)N1CCC(c2ccccc2C(F)(F)F)CC1]

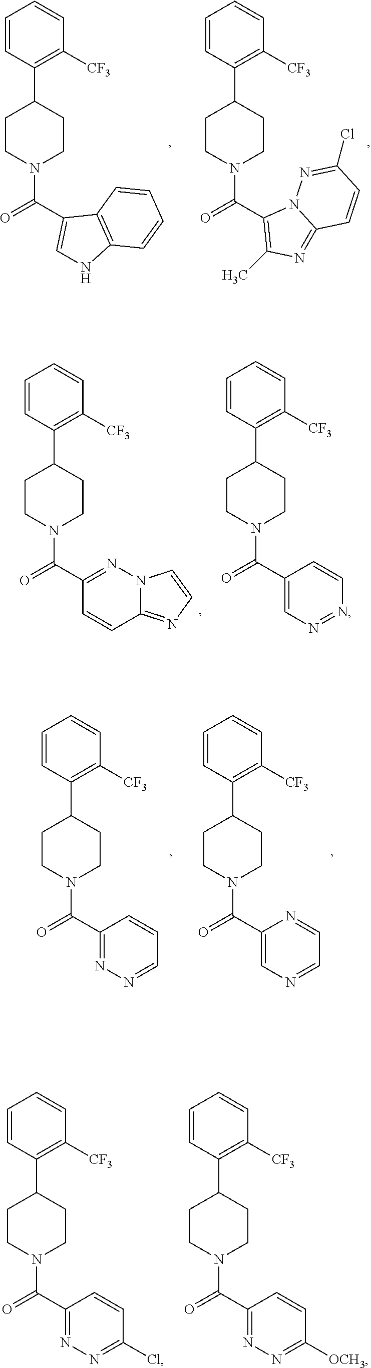

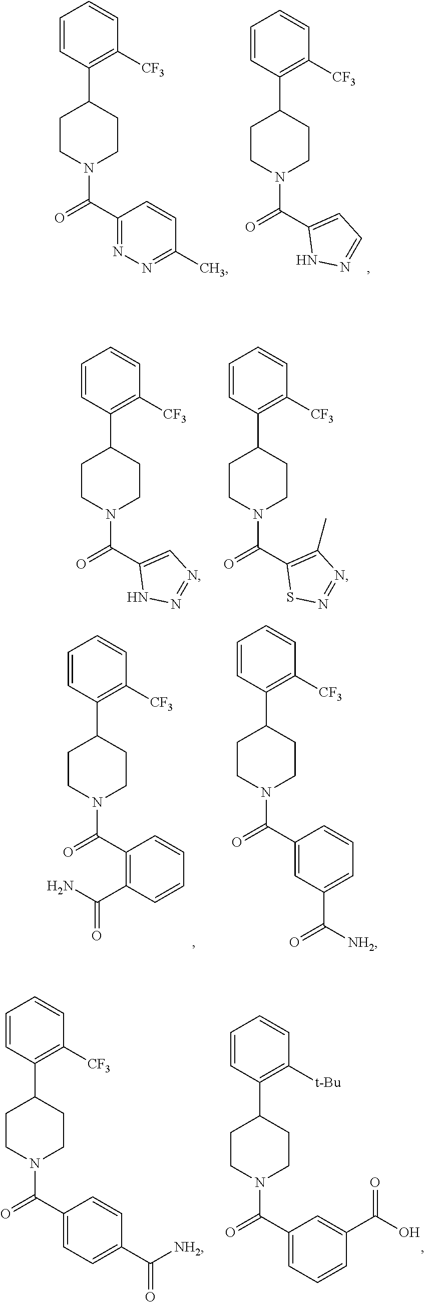

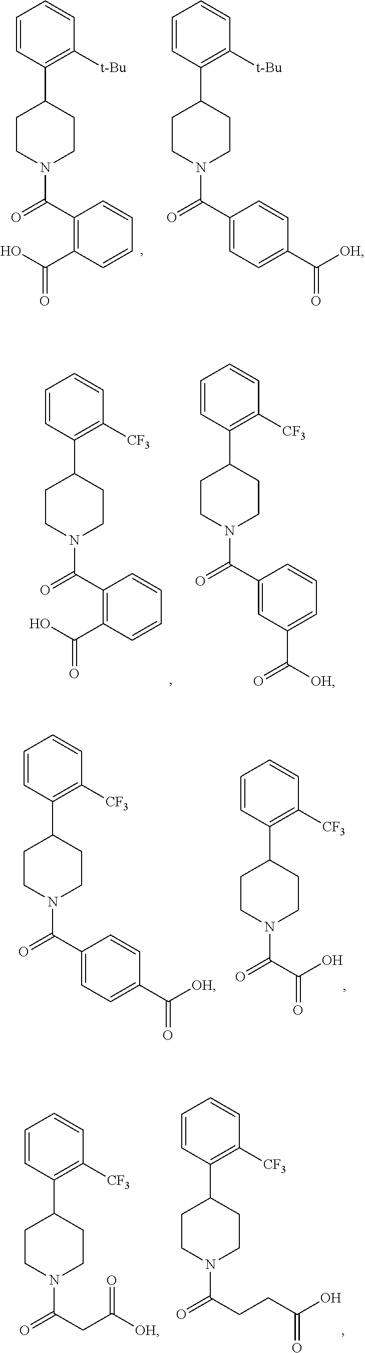

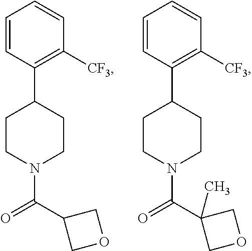

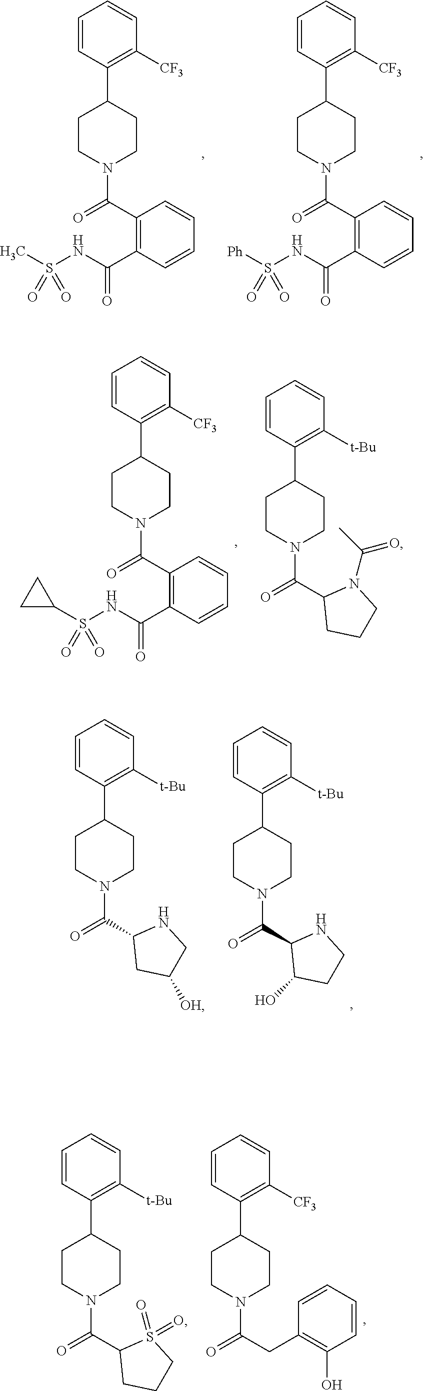

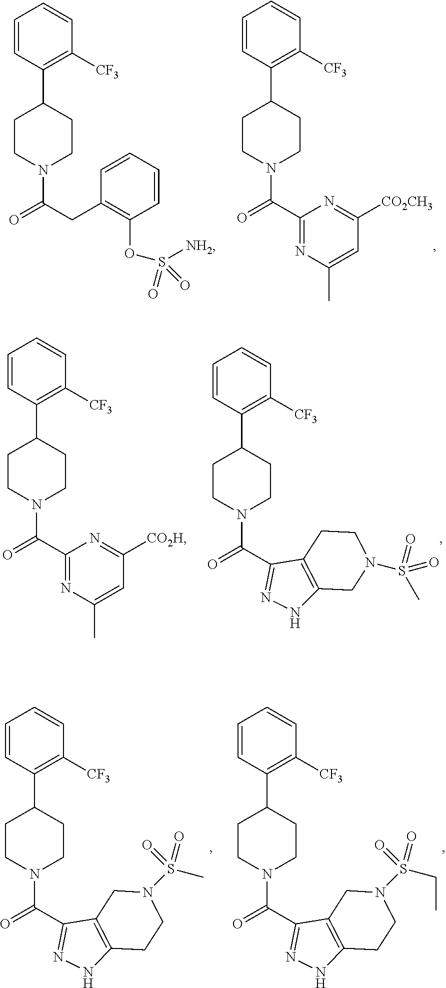

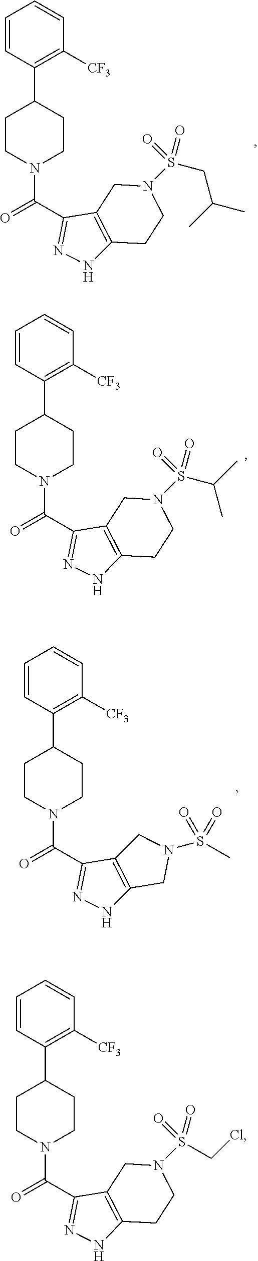 [SMILES: CC(C)CS(=O)(=O)N1CCc2[nH]nc(C(=O)N3CCC(c4ccccc4C(F)(F)F)CC3)c2C1 ; CC(C)S(=O)(=O)N1CCc2[nH]nc(C(=O)N3CCC(c4ccccc4C(F)(F)F)CC3)c2C1 ; CS(=O)(=O)N1Cc2[nH]nc(C(=O)N3CCC(c4ccccc4C(F)(F)F)CC3)c2C1 ; O=C(c1n[nH]c2c1CN(S(=O)(=O)CCl)CC2)N1CCC(c2ccccc2C(F)(F)F)CC1]

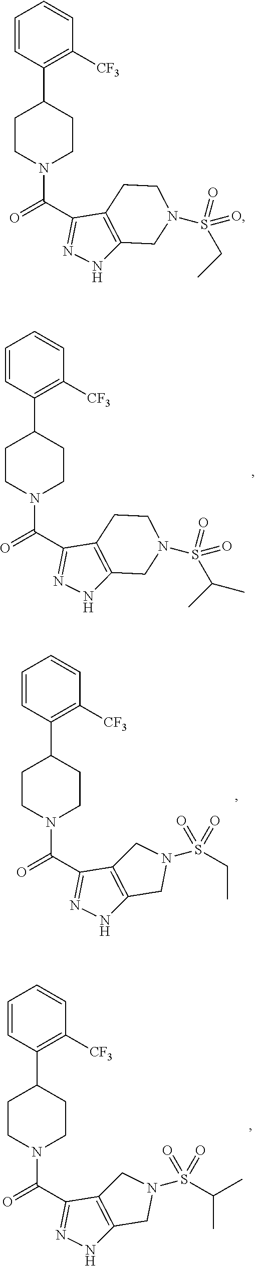 [SMILES: CCS(=O)(=O)N1CCc2c(C(=O)N3CCC(c4ccccc4C(F)(F)F)CC3)n[nH]c2C1 ; CC(C)S(=O)(=O)N1CCc2c(C(=O)N3CCC(c4ccccc4C(F)(F)F)CC3)n[nH]c2C1 ; CCS(=O)(=O)N1Cc2[nH]nc(C(=O)N3CCC(c4ccccc4C(F)(F)F)CC3)c2C1 ; CC(C)S(=O)(=O)N1Cc2[nH]nc(C(=O)N3CCC(c4ccccc4C(F)(F)F)CC3)c2C1]

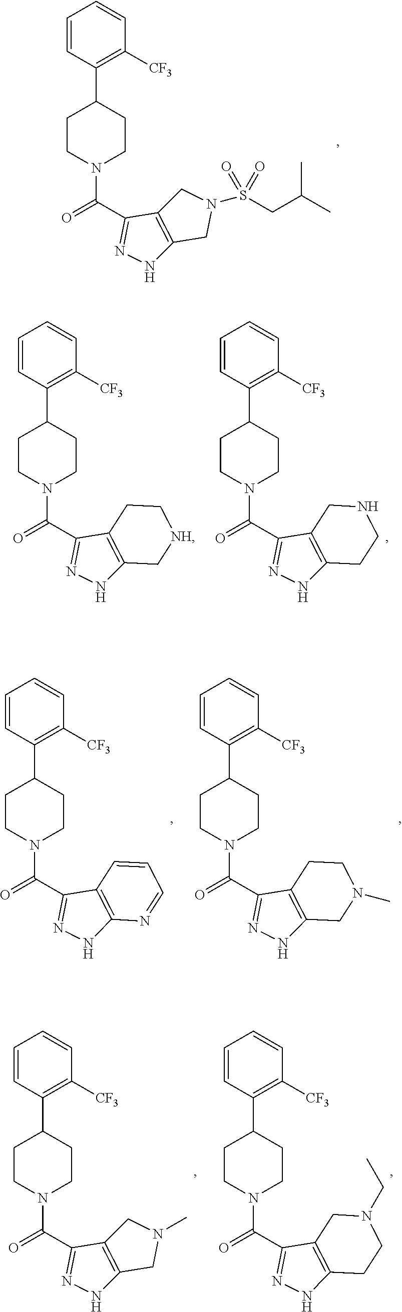

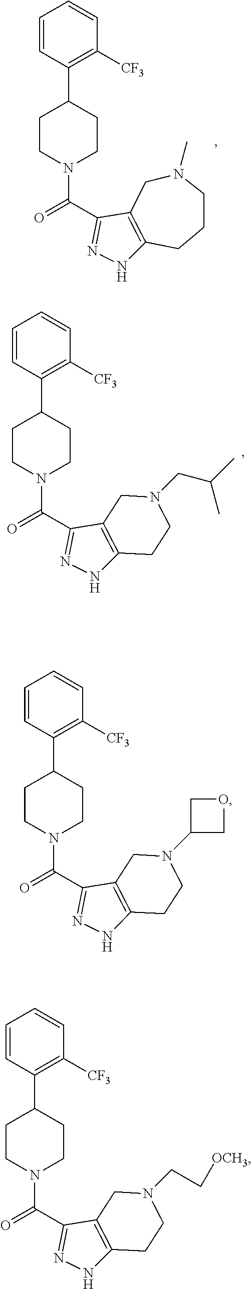 [SMILES: CN1CCCc2[nH]nc(C(=O)N3CCC(c4ccccc4C(F)(F)F)CC3)c2C1 ; CC(C)CN1CCc2[nH]nc(C(=O)N3CCC(c4ccccc4C(F)(F)F)CC3)c2C1 ; O=C(c1n[nH]c2c1CN(C1COC1)CC2)N1CCC(c2ccccc2C(F)(F)F)CC1 ; COCCN1CCc2[nH]nc(C(=O)N3CCC(c4ccccc4C(F)(F)F)CC3)c2C1]

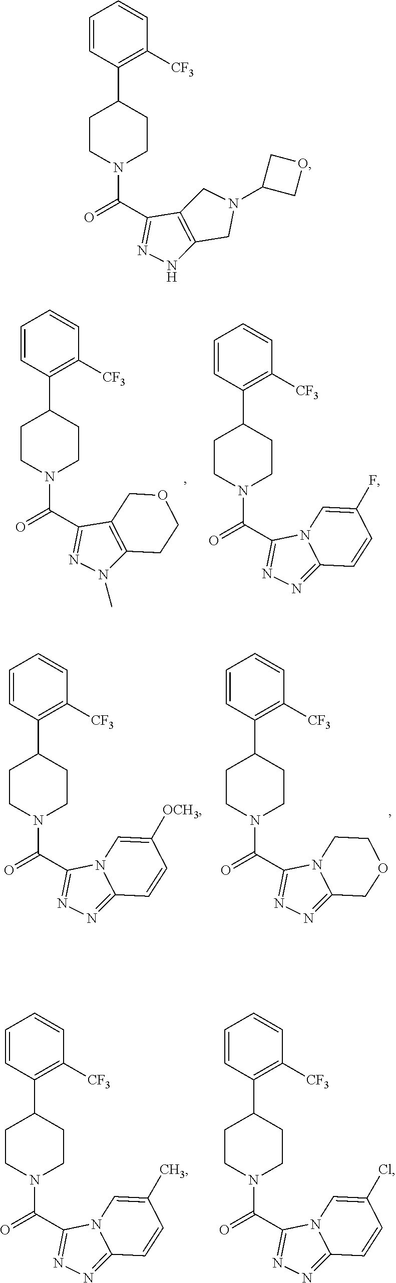

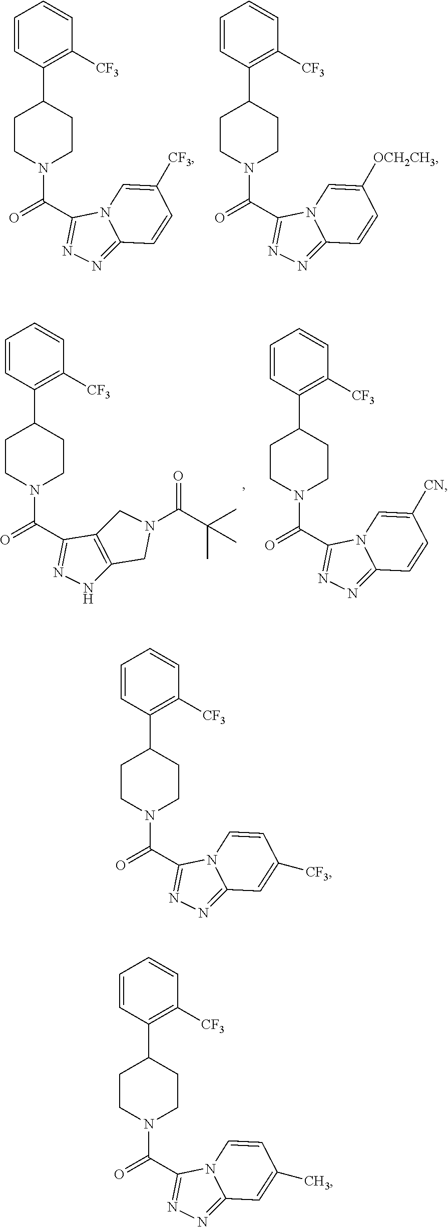

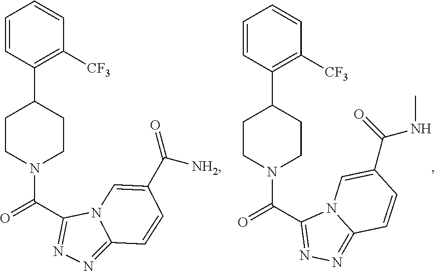 [SMILES: NC(=O)c1ccc2nnc(C(=O)N3CCC(c4ccccc4C(F)(F)F)CC3)n2c1 ; CNC(=O)c1ccc2nnc(C(=O)N3CCC(c4ccccc4C(F)(F)F)CC3)n2c1]

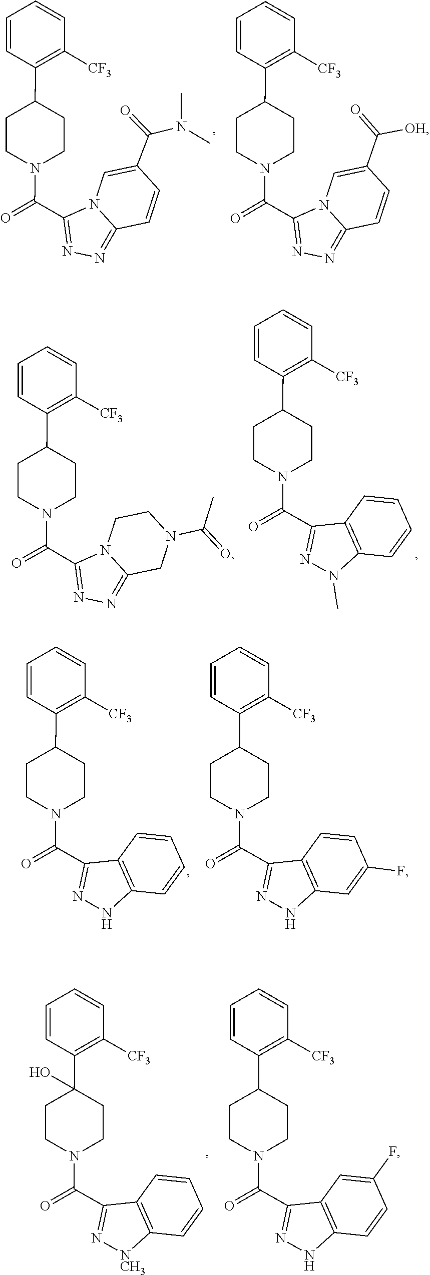

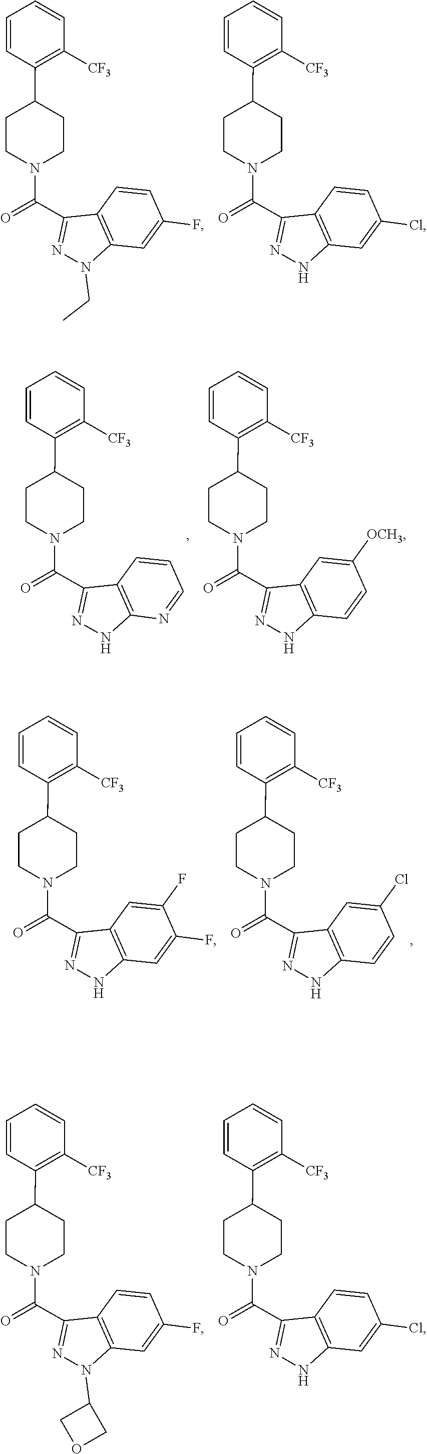

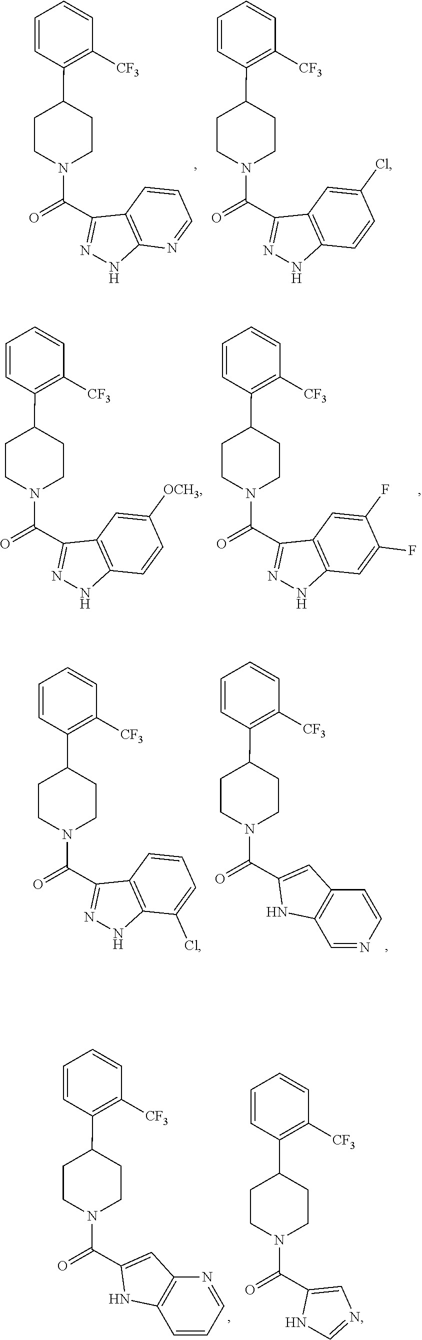

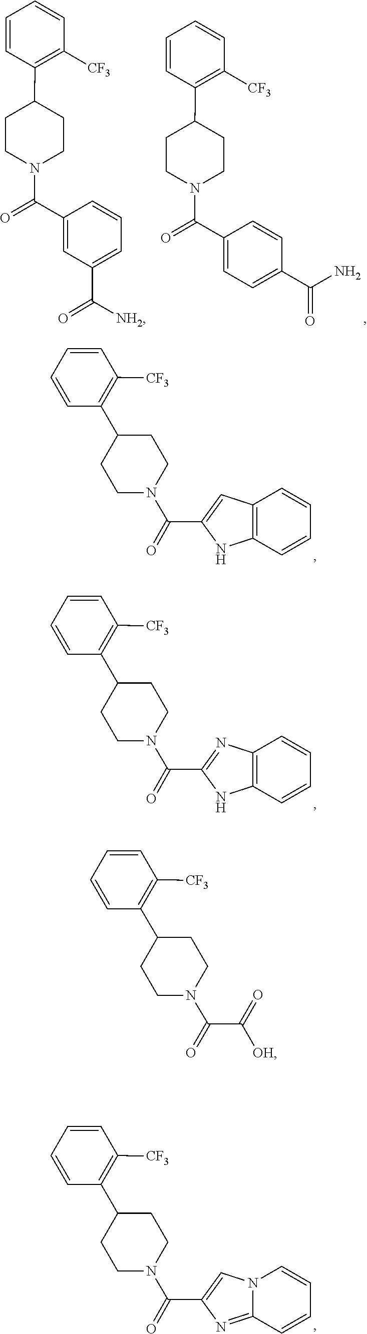

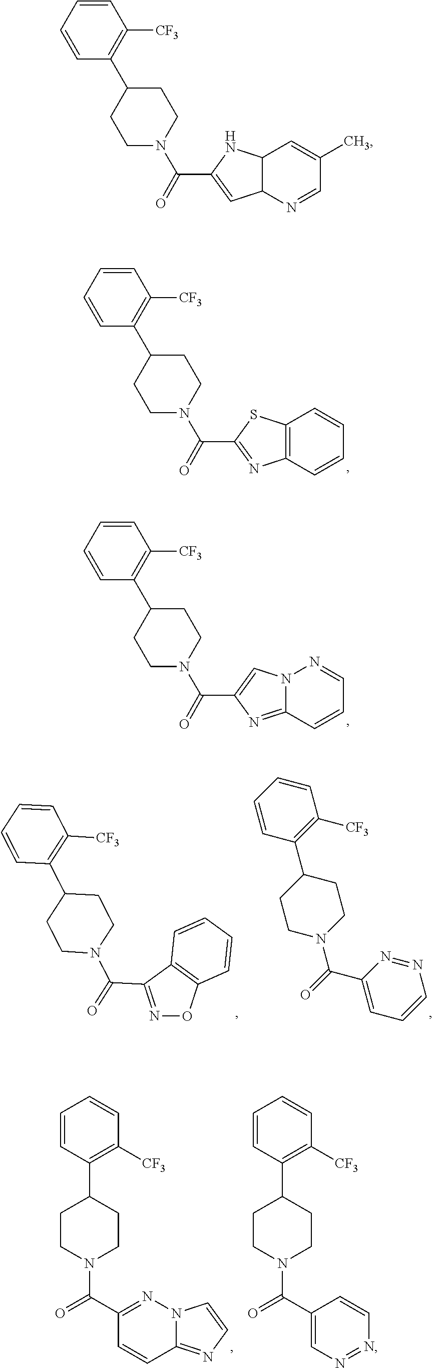

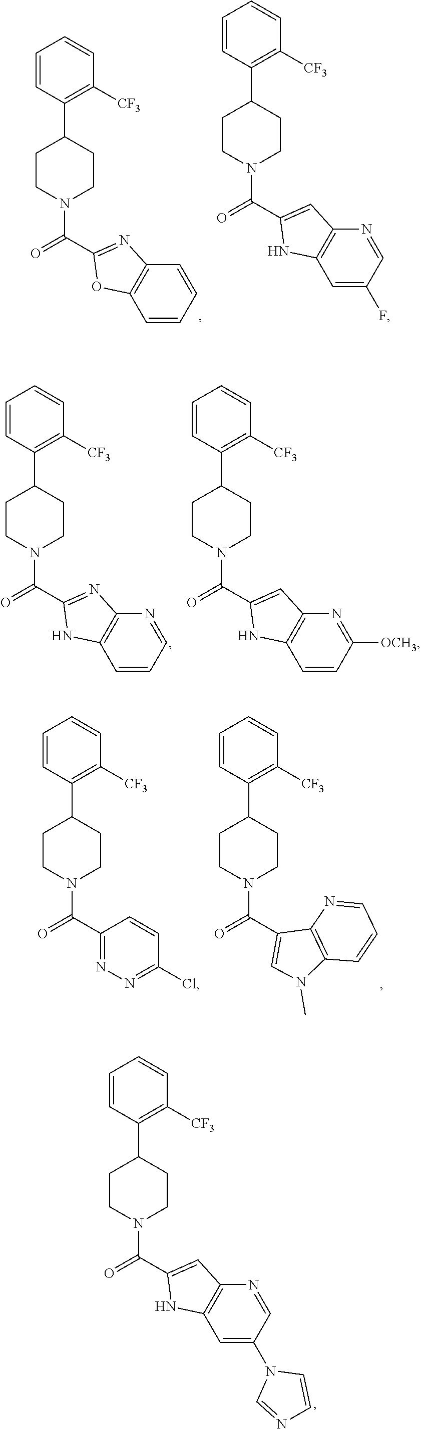

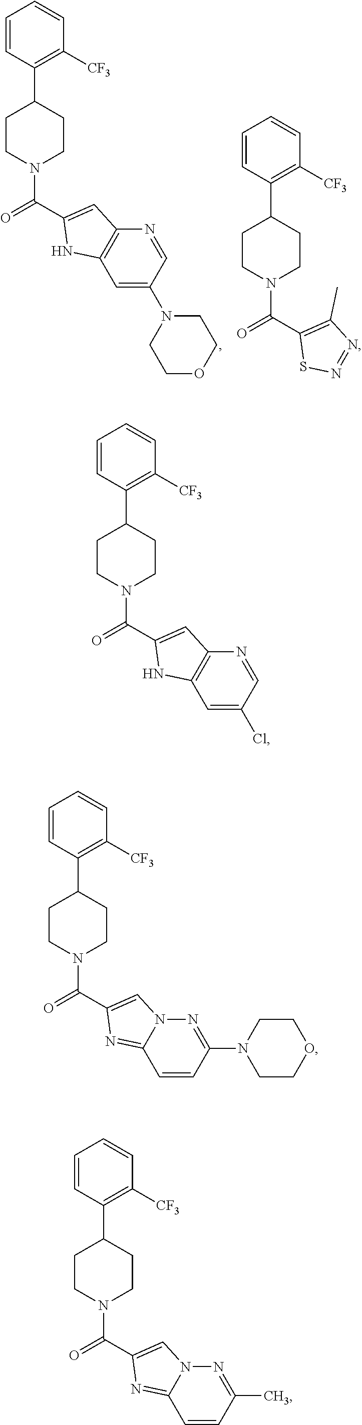

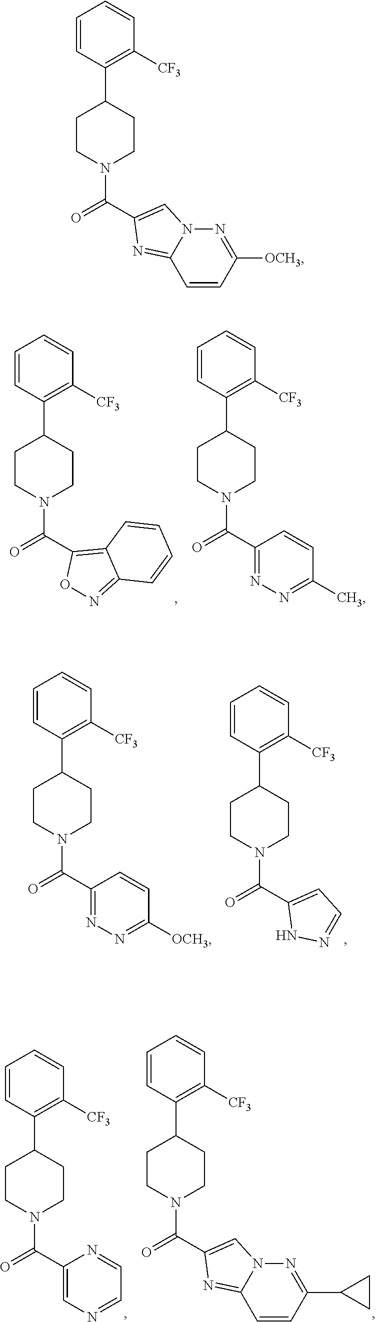

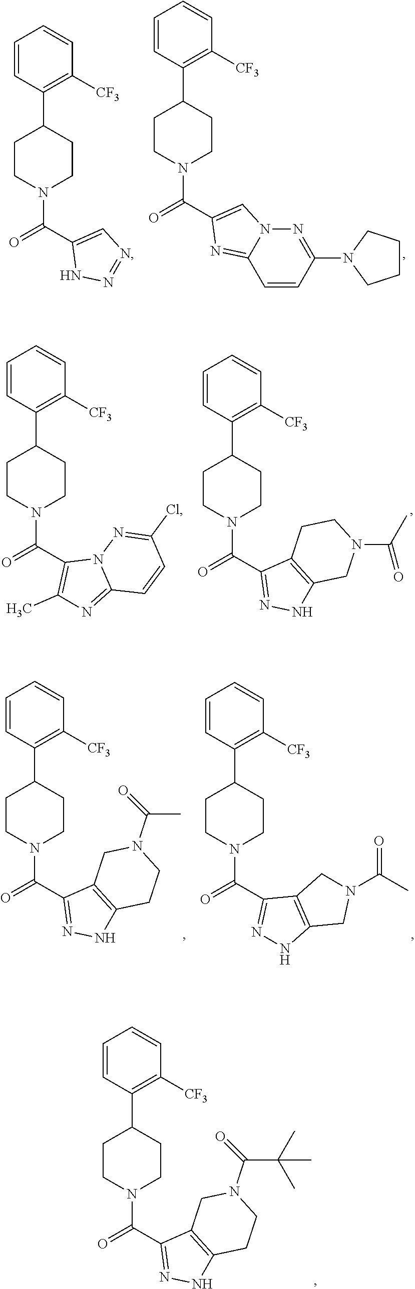

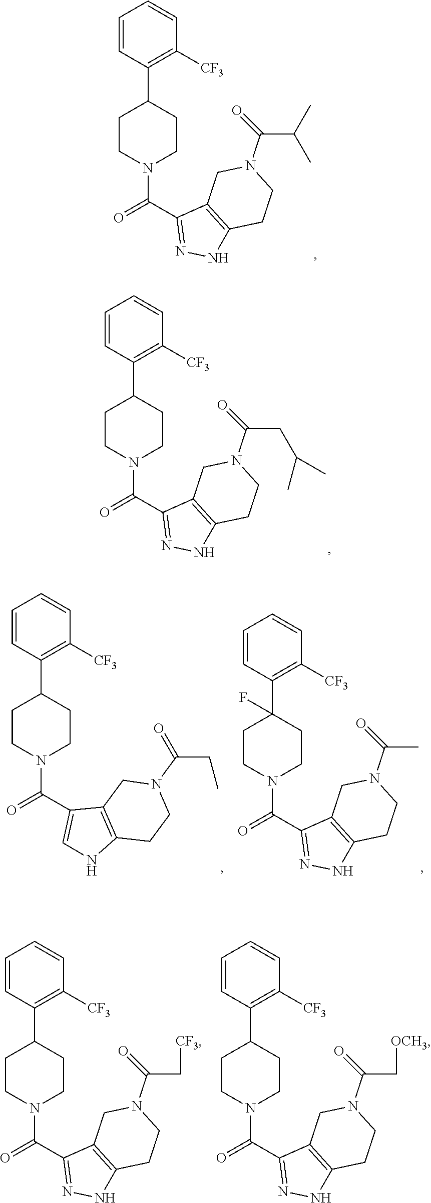

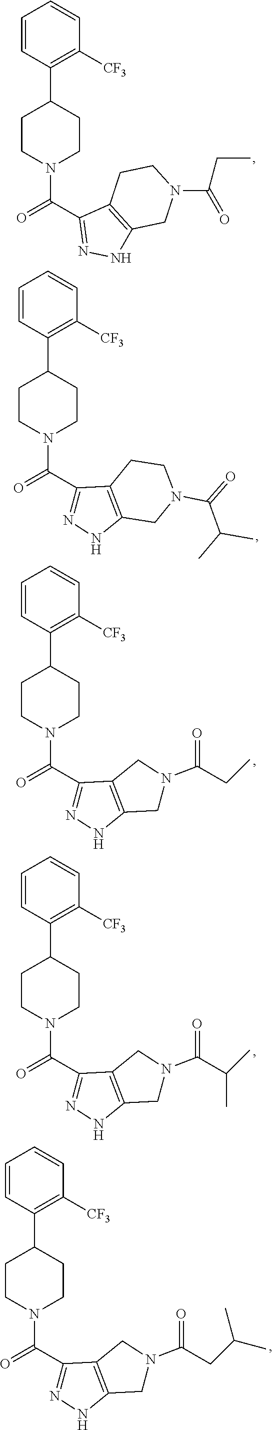

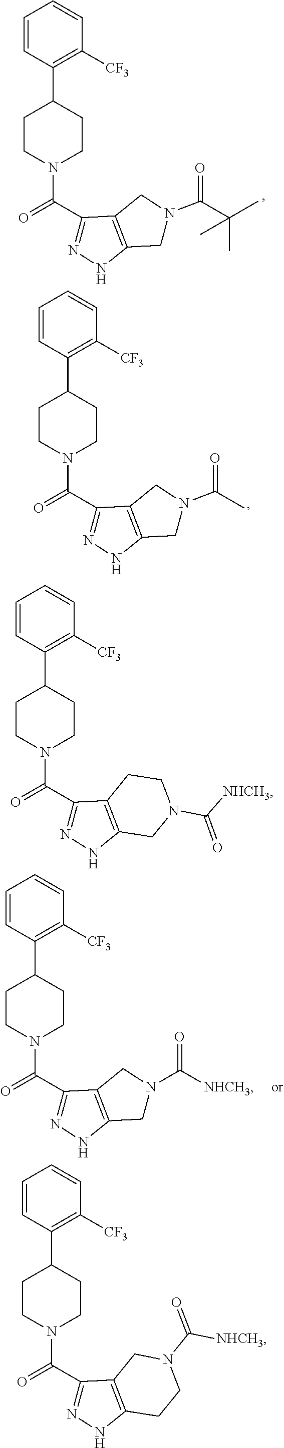

or a pharmaceutically acceptable salt thereof.

In some embodiments of the method, the selective RBP4 antagonist is

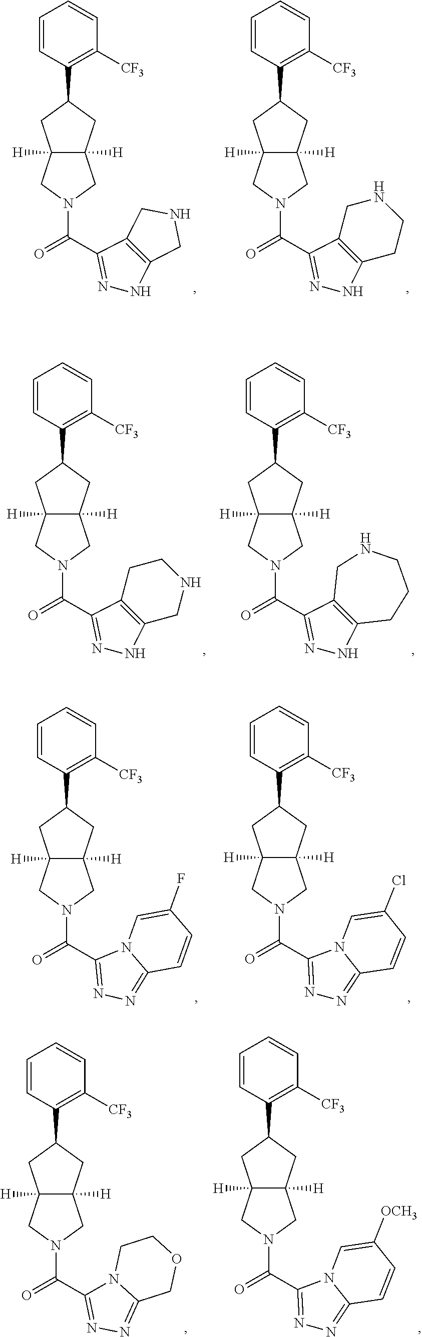

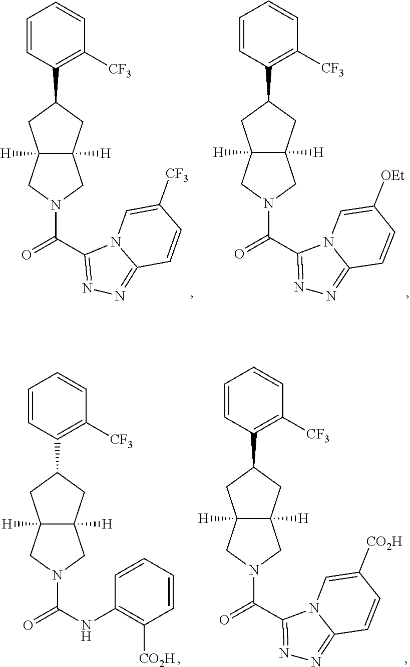 [SMILES: O=C(c1nnc2ccc(C(F)(F)F)cn12)N1C[C@H]2C[C@@H](c3ccccc3C(F)(F)F)C[C@H]2C1 ; CCOc1ccc2nnc(C(=O)N3C[C@H]4C[C@@H](c5ccccc5C(F)(F)F)C[C@H]4C3)n2c1 ; O=C(O)c1ccccc1NC(=O)N1C[C@H]2C[C@H](c3ccccc3C(F)(F)F)C[C@H]2C1 ; O=C(O)c1ccc2nnc(C(=O)N3C[C@H]4C[C@@H](c5ccccc5C(F)(F)F)C[C@H]4C3)n2c1]

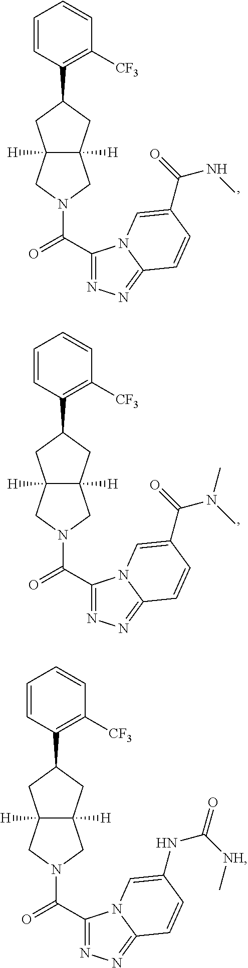 [SMILES: CNC(=O)c1ccc2nnc(C(=O)N3C[C@H]4C[C@@H](c5ccccc5C(F)(F)F)C[C@H]4C3)n2c1 ; CN(C)C(=O)c1ccc2nnc(C(=O)N3C[C@H]4C[C@@H](c5ccccc5C(F)(F)F)C[C@H]4C3)n2c1 ; CNC(=O)Nc1ccc2nnc(C(=O)N3C[C@H]4C[C@@H](c5ccccc5C(F)(F)F)C[C@H]4C3)n2c1]

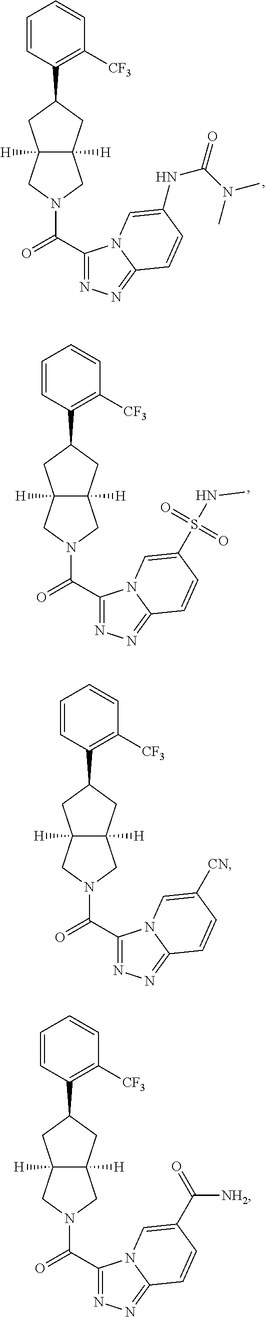 [SMILES: CN(C)C(=O)Nc1ccc2nnc(C(=O)N3C[C@H]4C[C@@H](c5ccccc5C(F)(F)F)C[C@H]4C3)n2c1 ; CNS(=O)(=O)c1ccc2nnc(C(=O)N3C[C@H]4C[C@@H](c5ccccc5C(F)(F)F)C[C@H]4C3)n2c1 ; N#Cc1ccc2nnc(C(=O)N3C[C@H]4C[C@@H](c5ccccc5C(F)(F)F)C[C@H]4C3)n2c1 ; NC(=O)c1ccc2nnc(C(=O)N3C[C@H]4C[C@@H](c5ccccc5C(F)(F)F)C[C@H]4C3)n2c1]

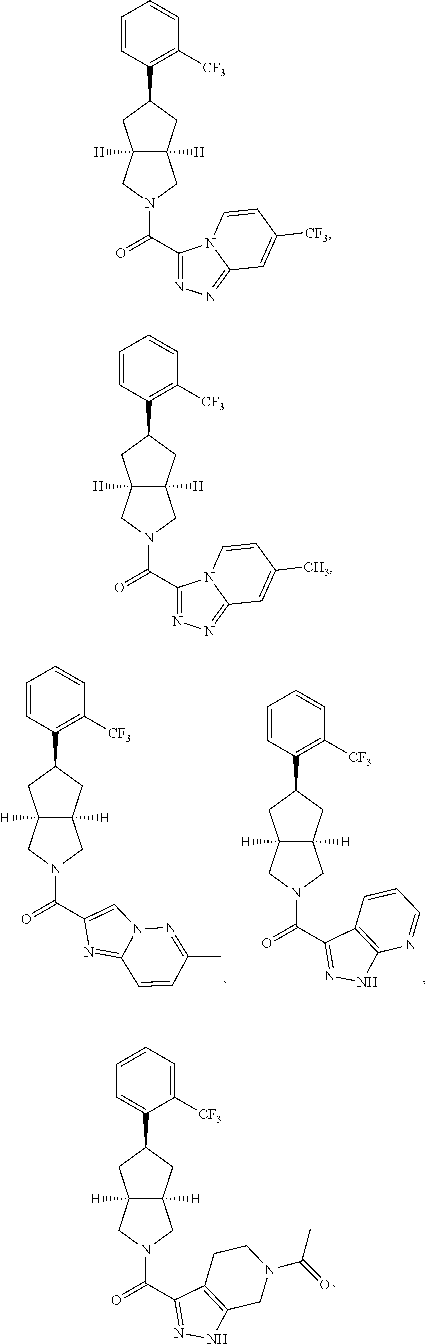 [SMILES: O=C(c1nnc2cc(C(F)(F)F)ccn12)N1C[C@H]2C[C@@H](c3ccccc3C(F)(F)F)C[C@H]2C1 ; Cc1ccn2c(C(=O)N3C[C@H]4C[C@@H](c5ccccc5C(F)(F)F)C[C@H]4C3)nnc2c1 ; Cc1ccc2nc(C(=O)N3C[C@H]4C[C@@H](c5ccccc5C(F)(F)F)C[C@H]4C3)cn2n1 ; O=C(c1n[nH]c2ncccc12)N1C[C@H]2C[C@@H](c3ccccc3C(F)(F)F)C[C@H]2C1 ; CC(=O)N1CCc2c(C(=O)N3C[C@H]4C[C@@H](c5ccccc5C(F)(F)F)C[C@H]4C3)n[nH]c2C1]

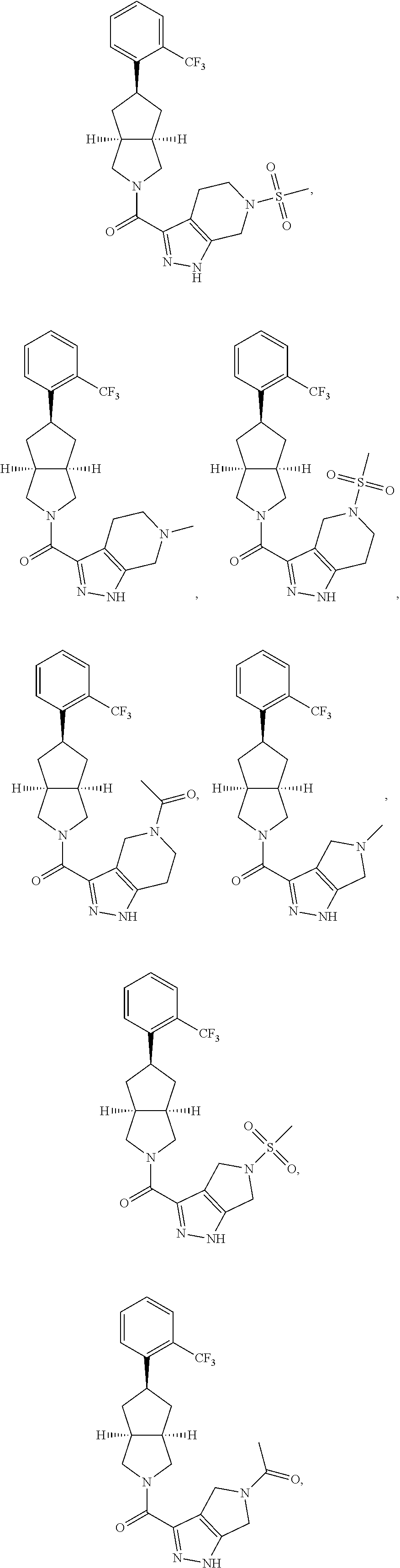

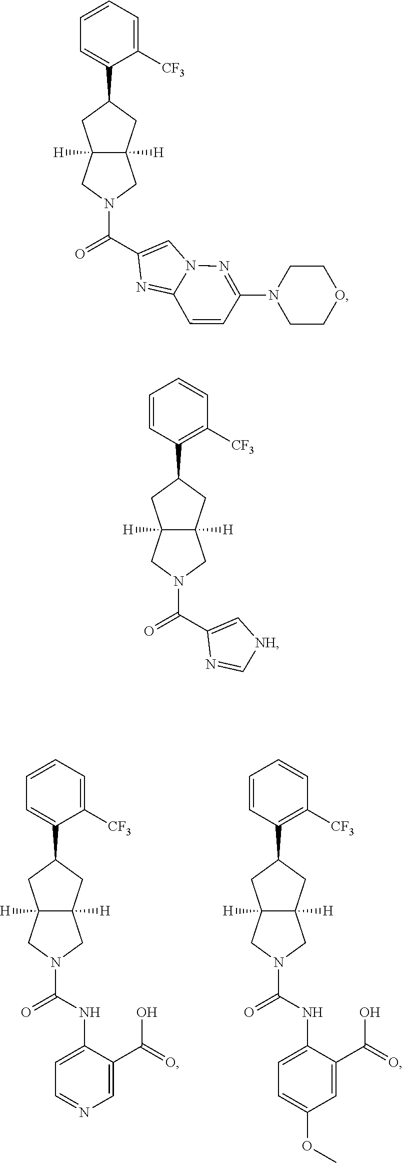 [SMILES: O=C(c1cn2nc(N3CCOCC3)ccc2n1)N1C[C@H]2C[C@@H](c3ccccc3C(F)(F)F)C[C@H]2C1 ; O=C(c1c[nH]cn1)N1C[C@H]2C[C@@H](c3ccccc3C(F)(F)F)C[C@H]2C1 ; O=C(O)c1cnccc1NC(=O)N1C[C@H]2C[C@@H](c3ccccc3C(F)(F)F)C[C@H]2C1 ; COc1ccc(NC(=O)N2C[C@H]3C[C@@H](c4ccccc4C(F)(F)F)C[C@H]3C2)c(C(=O)O)c1]

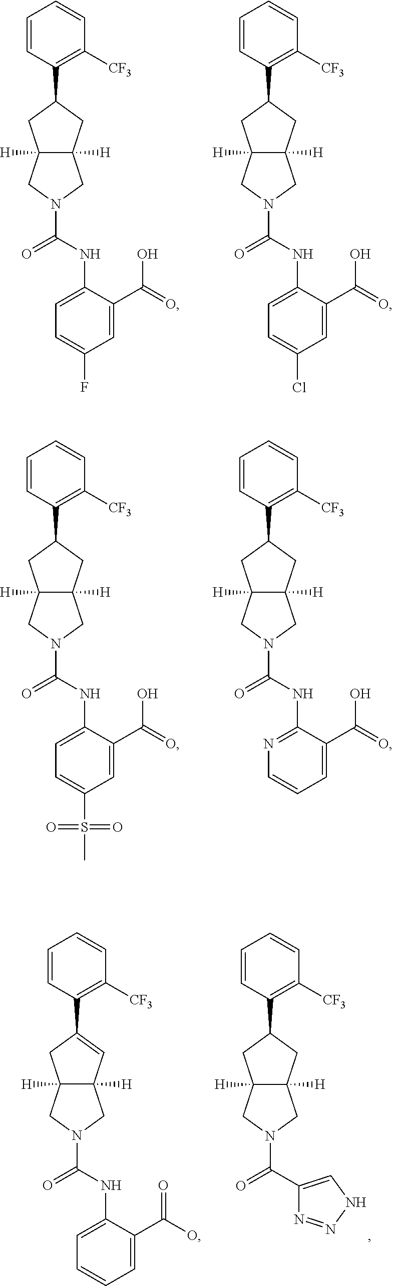

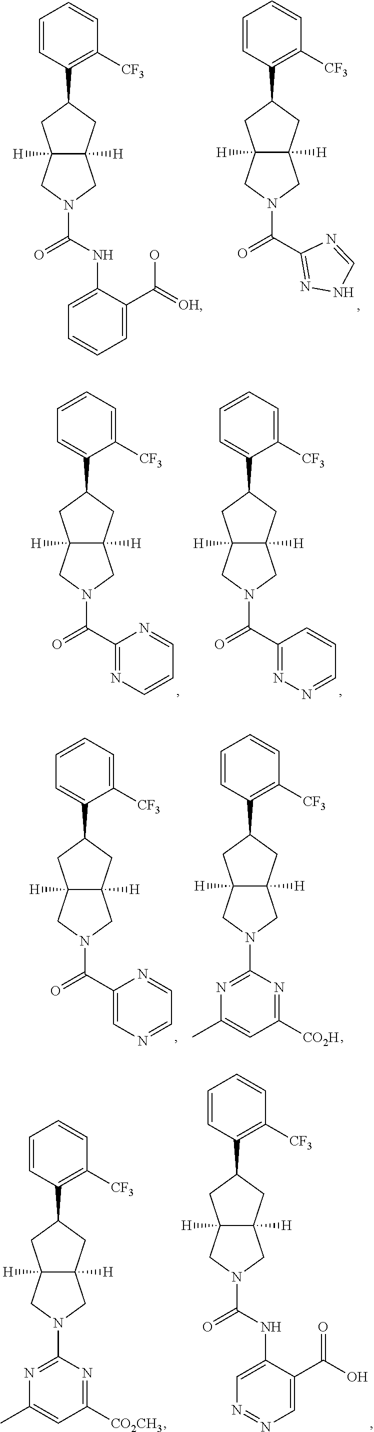

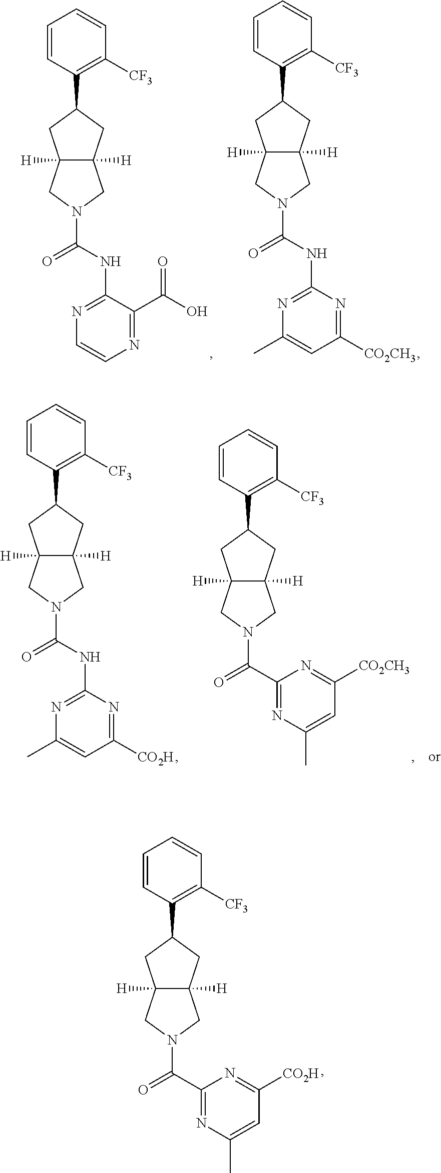 [SMILES: O=C(O)c1nccnc1NC(=O)N1C[C@H]2C[C@@H](c3ccccc3C(F)(F)F)C[C@H]2C1 ; COC(=O)c1cc(C)nc(NC(=O)N2C[C@H]3C[C@@H](c4ccccc4C(F)(F)F)C[C@H]3C2)n1 ; Cc1cc(C(=O)O)nc(NC(=O)N2C[C@H]3C[C@@H](c4ccccc4C(F)(F)F)C[C@H]3C2)n1 ; COC(=O)c1cc(C)nc(C(=O)N2C[C@H]3C[C@@H](c4ccccc4C(F)(F)F)C[C@H]3C2)n1 ; Cc1cc(C(=O)O)nc(C(=O)N2C[C@H]3C[C@@H](c4ccccc4C(F)(F)F)C[C@H]3C2)n1]

or a pharmaceutically acceptable salt thereof.

In some embodiments of the method, the selective RBP4 antagonist is

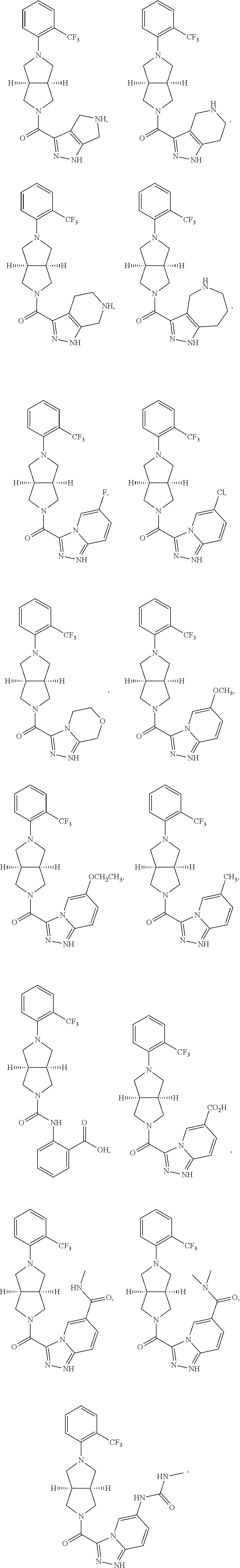

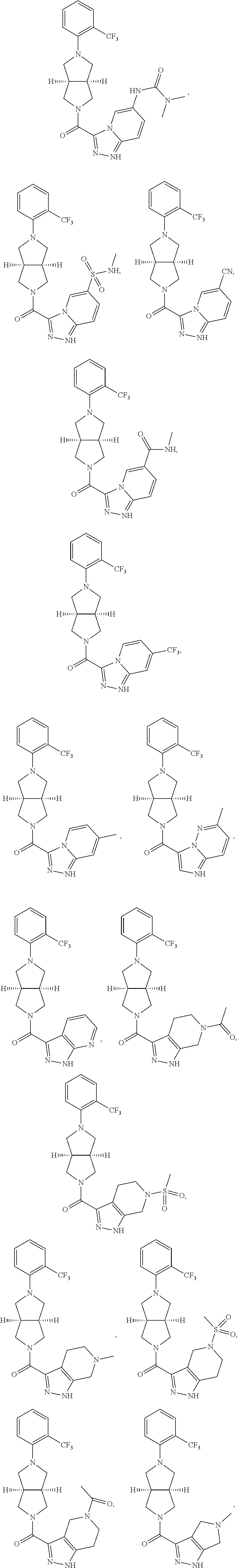

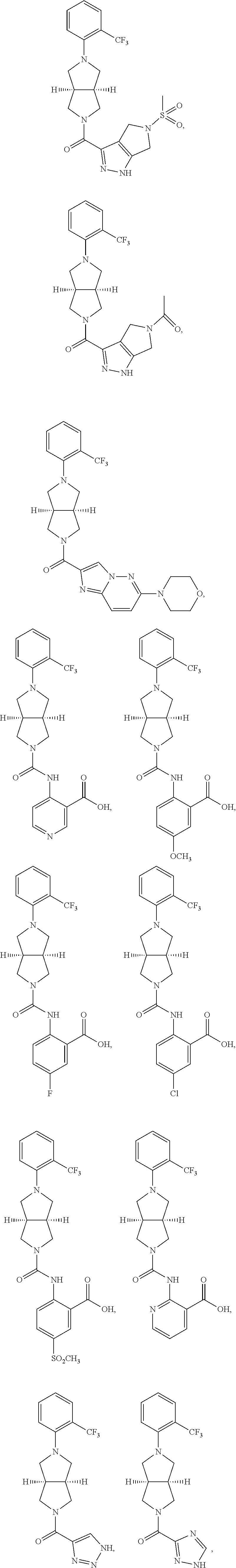

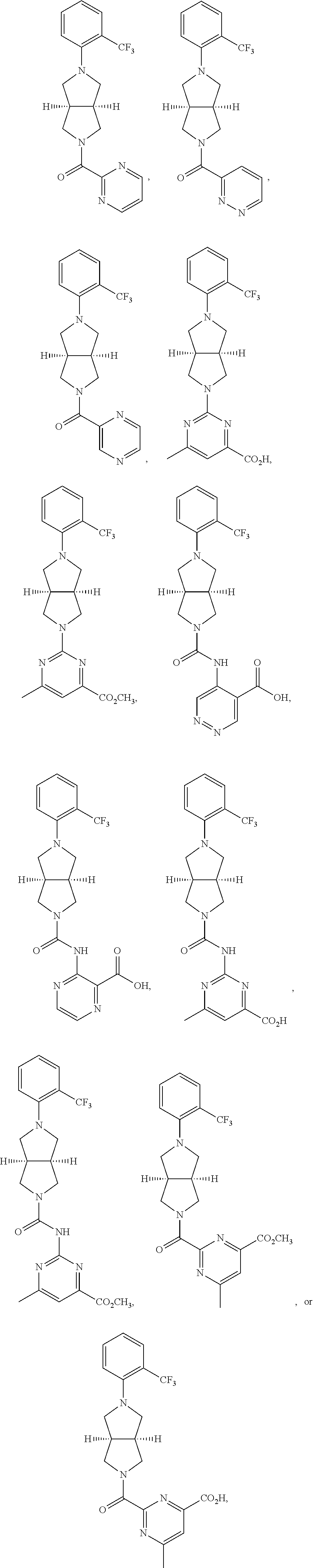

or a pharmaceutically acceptable salt thereof.

In some embodiments of the method, the selective RBP4 antagonist is

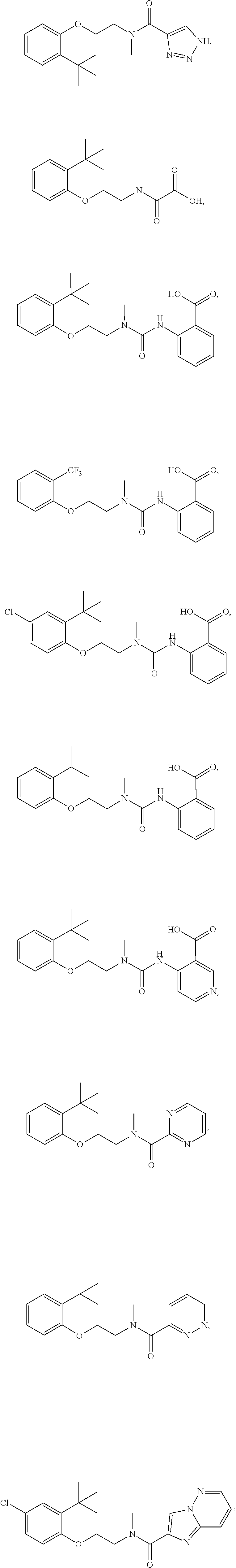

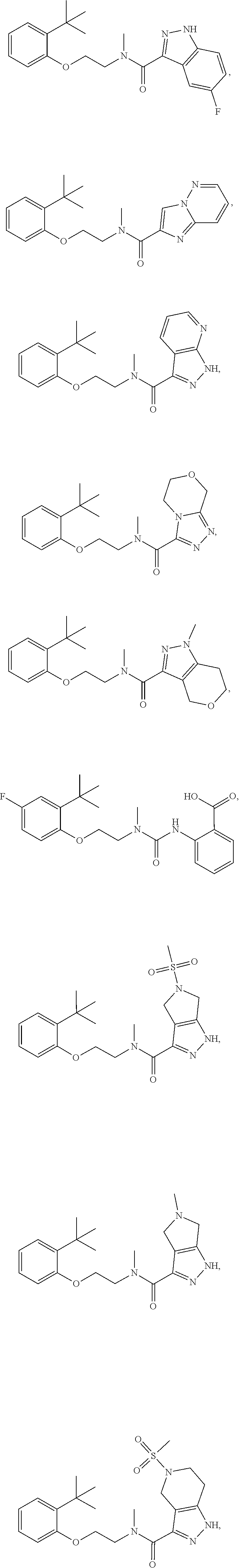

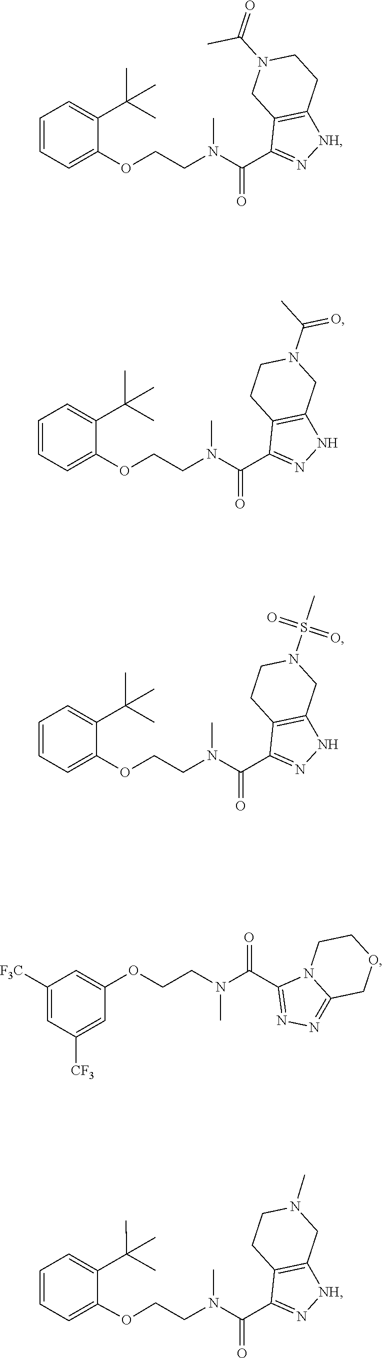 [SMILES: CC(=O)N1CCc2[nH]nc(C(=O)N(C)CCOc3ccccc3C(C)(C)C)c2C1 ; CC(=O)N1CCc2c(C(=O)N(C)CCOc3ccccc3C(C)(C)C)n[nH]c2C1 ; CN(CCOc1ccccc1C(C)(C)C)C(=O)c1n[nH]c2c1CCN(S(C)(=O)=O)C2 ; CN(CCOc1cc(C(F)(F)F)cc(C(F)(F)F)c1)C(=O)c1nnc2n1CCOC2 ; CN1CCc2c(C(=O)N(C)CCOc3ccccc3C(C)(C)C)n[nH]c2C1]

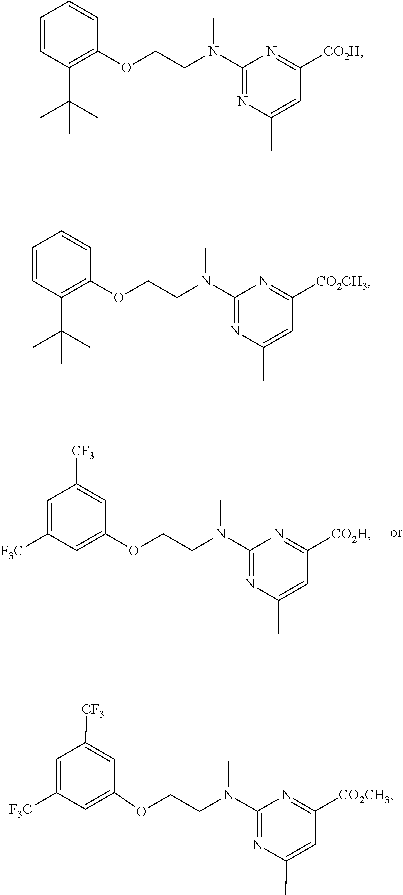 [SMILES: Cc1cc(C(=O)O)nc(N(C)CCOc2ccccc2C(C)(C)C)n1 ; COC(=O)c1cc(C)nc(N(C)CCOc2ccccc2C(C)(C)C)n1 ; Cc1cc(C(=O)O)nc(N(C)CCOc2cc(C(F)(F)F)cc(C(F)(F)F)c2)n1 ; "or" ; COC(=O)c1cc(C)nc(N(C)CCOc2cc(C(F)(F)F)cc(C(F)(F)F)c2)n1]

or a pharmaceutically acceptable salt thereof.

In some embodiments of the method, the selective RBP4 antagonist is

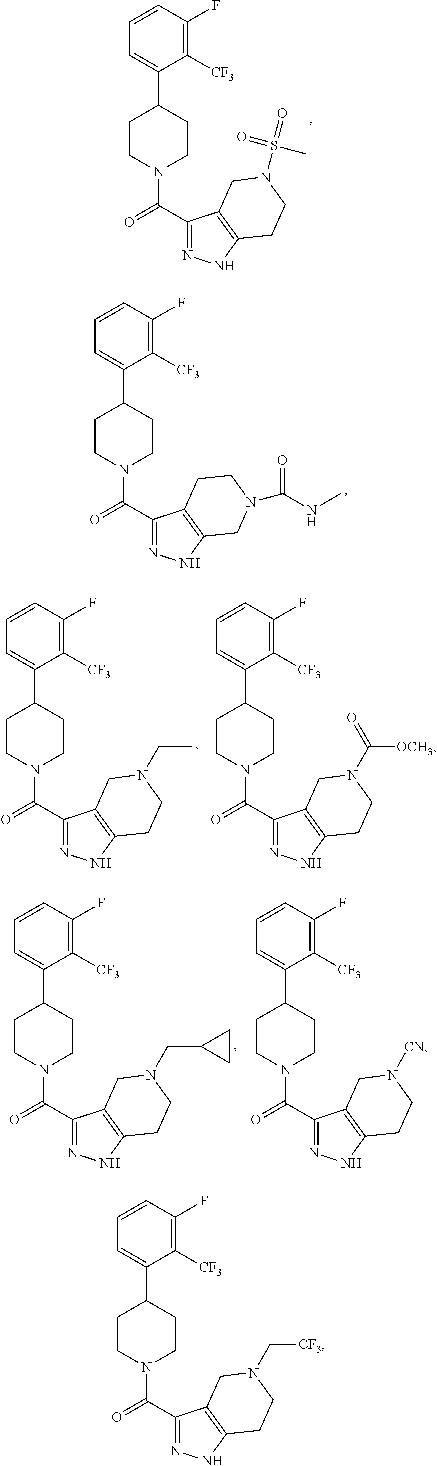

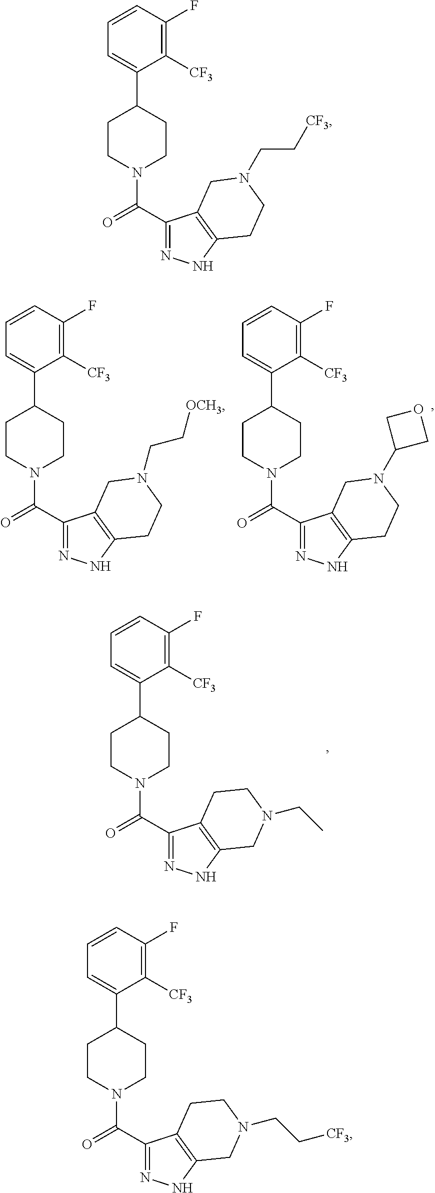 [SMILES: O=C(c1n[nH]c2c1CN(CCC(F)(F)F)CC2)N1CCC(c2cccc(F)c2C(F)(F)F)CC1 ; COCCN1CCc2[nH]nc(C(=O)N3CCC(c4cccc(F)c4C(F)(F)F)CC3)c2C1 ; O=C(c1n[nH]c2c1CN(C1COC1)CC2)N1CCC(c2cccc(F)c2C(F)(F)F)CC1 ; CCN1CCc2c(C(=O)N3CCC(c4cccc(F)c4C(F)(F)F)CC3)n[nH]c2C1 ; O=C(c1n[nH]c2c1CCN(CCC(F)(F)F)C2)N1CCC(c2cccc(F)c2C(F)(F)F)CC1]

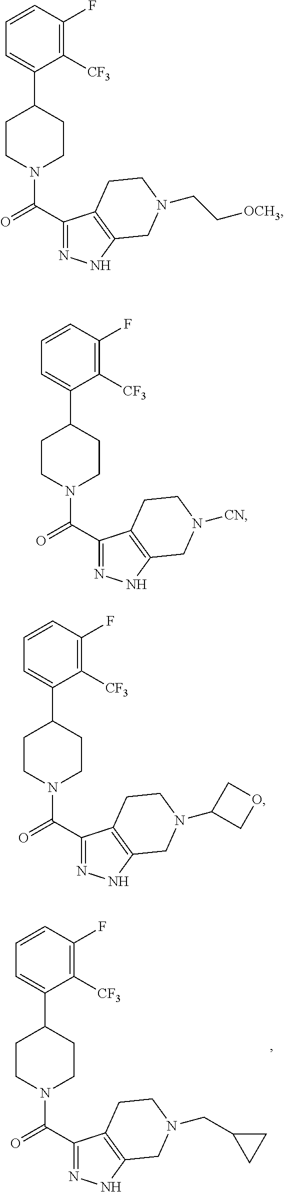

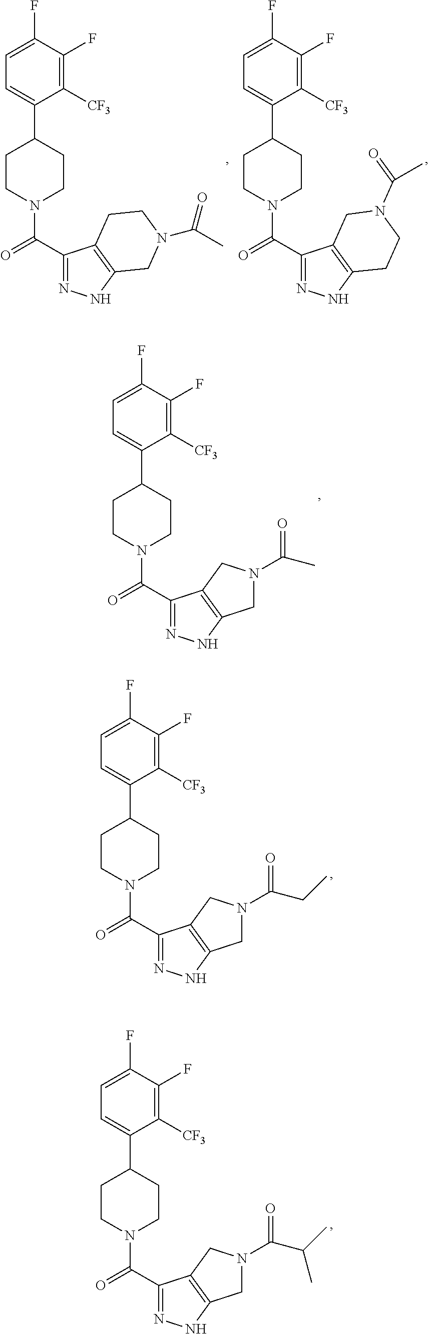

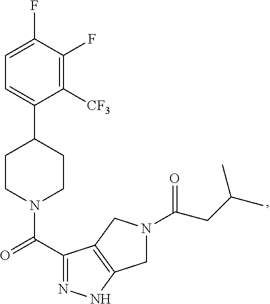 [SMILES: CC(C)CC(=O)N1Cc2[nH]nc(C(=O)N3CCC(c4ccc(F)c(F)c4C(F)(F)F)CC3)c2C1]

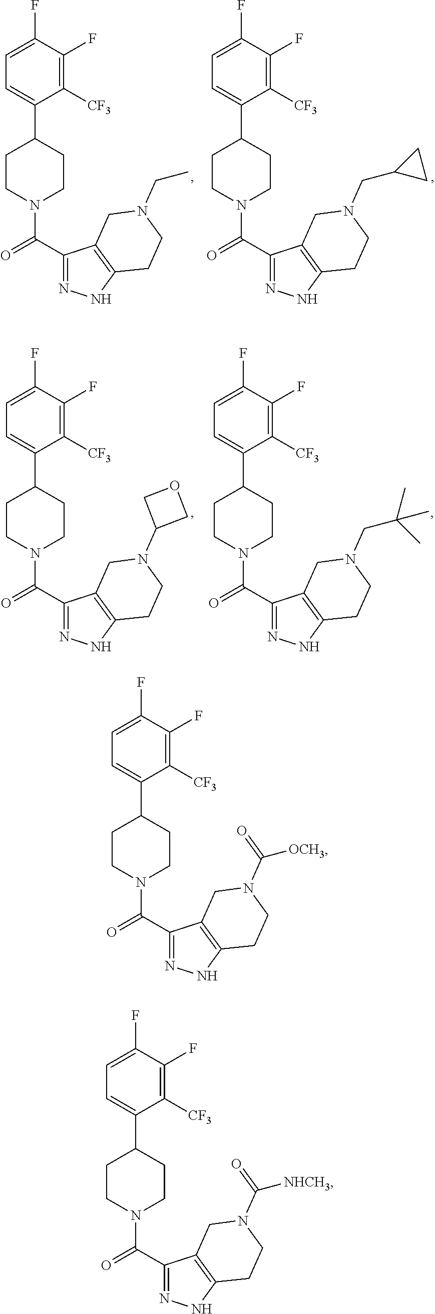

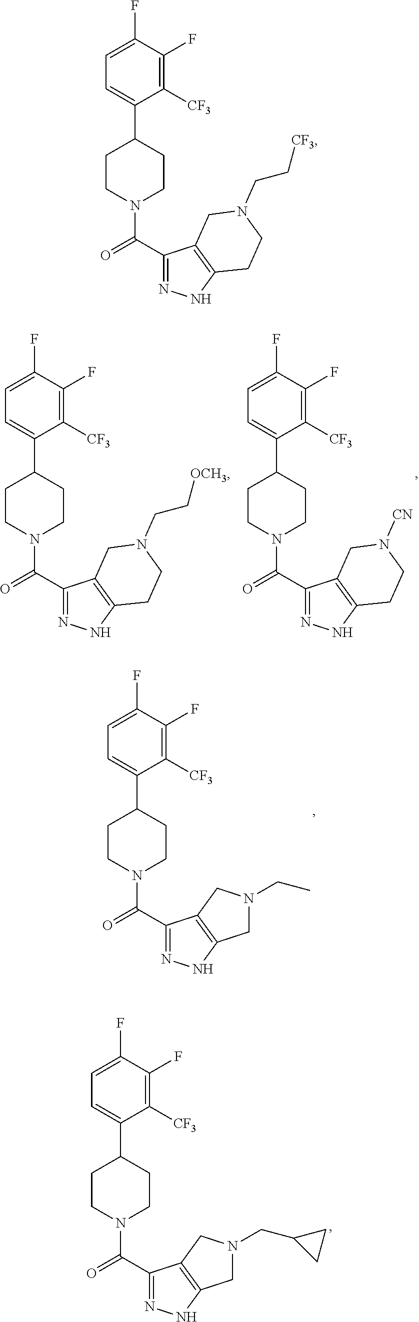 [SMILES: O=C(c1n[nH]c2c1CN(CCC(F)(F)F)CC2)N1CCC(c2ccc(F)c(F)c2C(F)(F)F)CC1 ; COCCN1CCc2[nH]nc(C(=O)N3CCC(c4ccc(F)c(F)c4C(F)(F)F)CC3)c2C1 ; N#CN1CCc2[nH]nc(C(=O)N3CCC(c4ccc(F)c(F)c4C(F)(F)F)CC3)c2C1 ; CCN1Cc2[nH]nc(C(=O)N3CCC(c4ccc(F)c(F)c4C(F)(F)F)CC3)c2C1 ; O=C(c1n[nH]c2c1CN(CC1CC1)C2)N1CCC(c2ccc(F)c(F)c2C(F)(F)F)CC1]

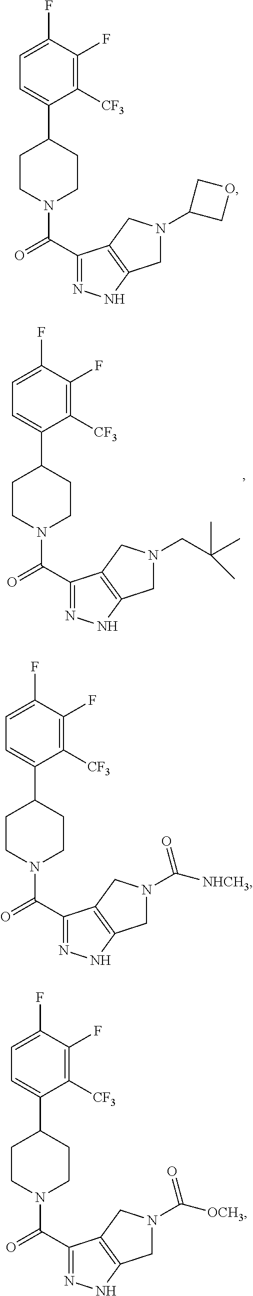 [SMILES: O=C(c1n[nH]c2c1CN(C1COC1)C2)N1CCC(c2ccc(F)c(F)c2C(F)(F)F)CC1 ; CC(C)(C)CN1Cc2[nH]nc(C(=O)N3CCC(c4ccc(F)c(F)c4C(F)(F)F)CC3)c2C1 ; CNC(=O)N1Cc2[nH]nc(C(=O)N3CCC(c4ccc(F)c(F)c4C(F)(F)F)CC3)c2C1 ; COC(=O)N1Cc2[nH]nc(C(=O)N3CCC(c4ccc(F)c(F)c4C(F)(F)F)CC3)c2C1]

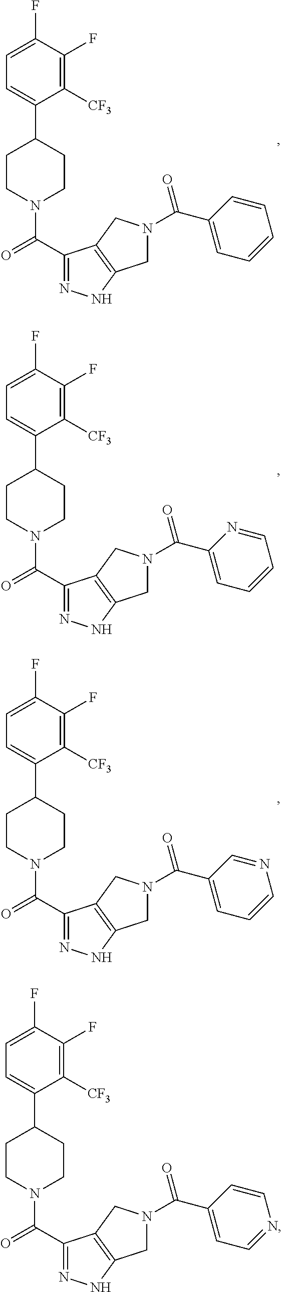 [SMILES: O=C(c1ccccc1)N1Cc2[nH]nc(C(=O)N3CCC(c4ccc(F)c(F)c4C(F)(F)F)CC3)c2C1 ; O=C(c1ccccn1)N1Cc2[nH]nc(C(=O)N3CCC(c4ccc(F)c(F)c4C(F)(F)F)CC3)c2C1 ; O=C(c1cccnc1)N1Cc2[nH]nc(C(=O)N3CCC(c4ccc(F)c(F)c4C(F)(F)F)CC3)c2C1 ; O=C(c1ccncc1)N1Cc2[nH]nc(C(=O)N3CCC(c4ccc(F)c(F)c4C(F)(F)F)CC3)c2C1]

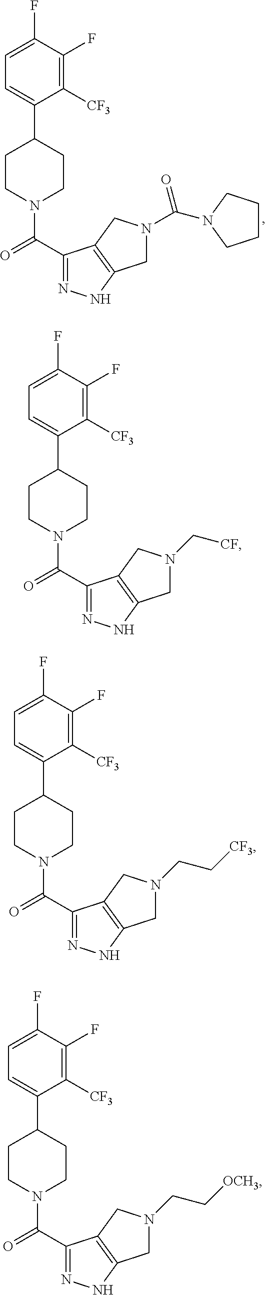

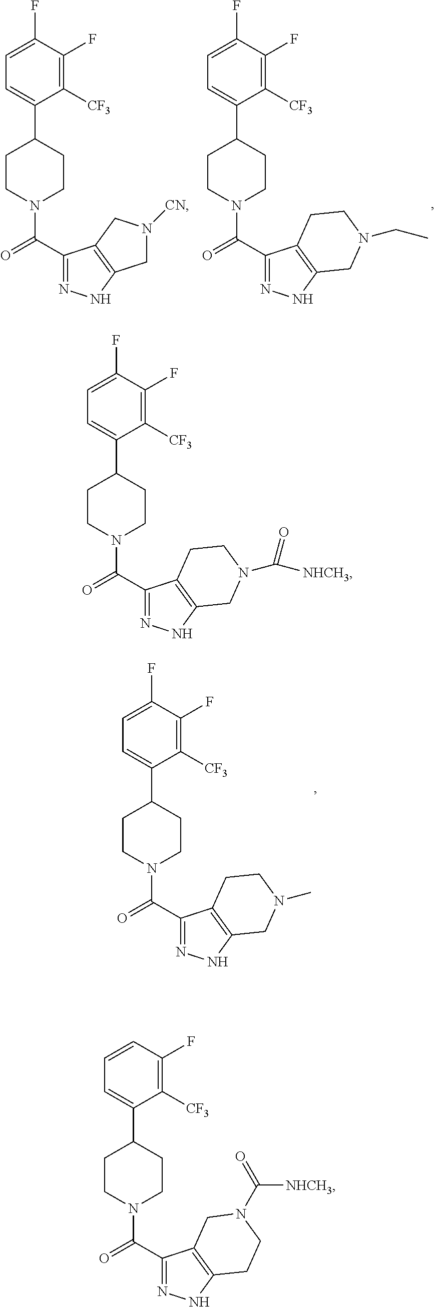

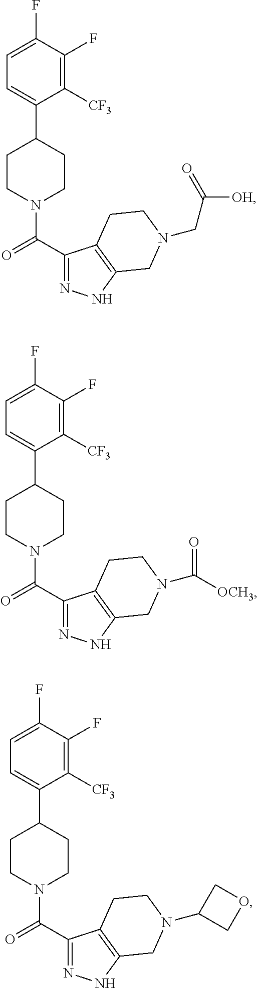 [SMILES: O=C(O)CN1CCc2c(C(=O)N3CCC(c4ccc(F)c(F)c4C(F)(F)F)CC3)n[nH]c2C1 ; COC(=O)N1CCc2c(C(=O)N3CCC(c4ccc(F)c(F)c4C(F)(F)F)CC3)n[nH]c2C1 ; O=C(c1n[nH]c2c1CCN(C1COC1)C2)N1CCC(c2ccc(F)c(F)c2C(F)(F)F)CC1]

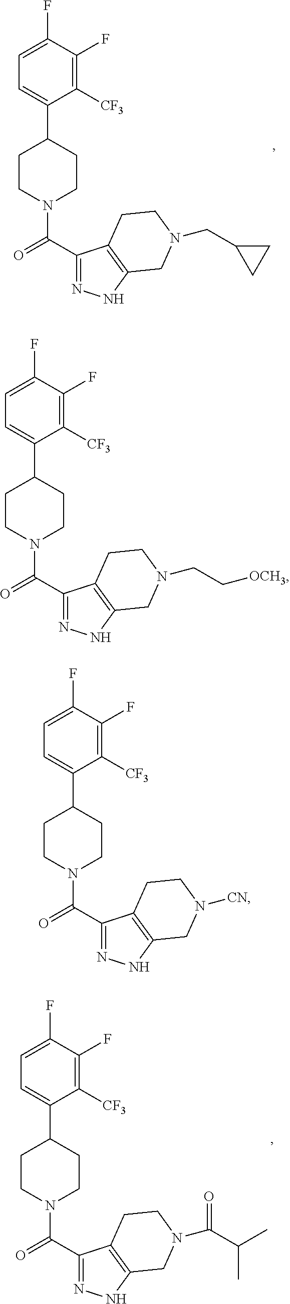

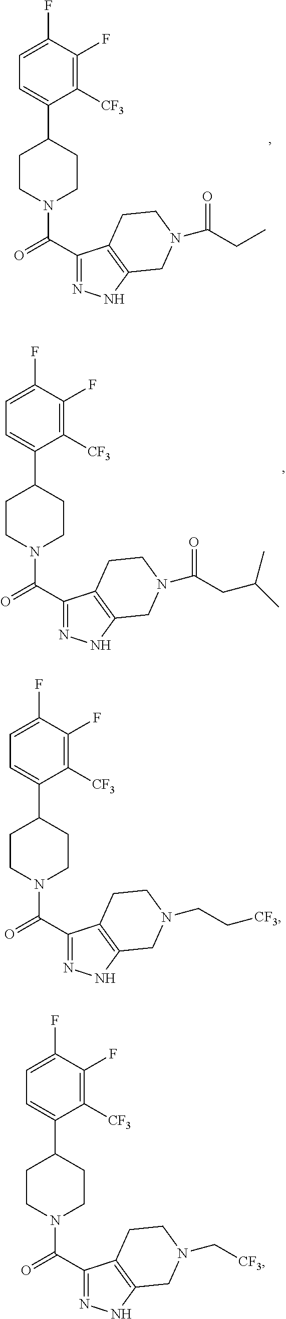 [SMILES: CCC(=O)N1CCc2c(C(=O)N3CCC(c4ccc(F)c(F)c4C(F)(F)F)CC3)n[nH]c2C1 ; CC(C)CC(=O)N1CCc2c(C(=O)N3CCC(c4ccc(F)c(F)c4C(F)(F)F)CC3)n[nH]c2C1 ; O=C(c1n[nH]c2c1CCN(CCC(F)(F)F)C2)N1CCC(c2ccc(F)c(F)c2C(F)(F)F)CC1 ; O=C(c1n[nH]c2c1CCN(CC(F)(F)F)C2)N1CCC(c2ccc(F)c(F)c2C(F)(F)F)CC1]

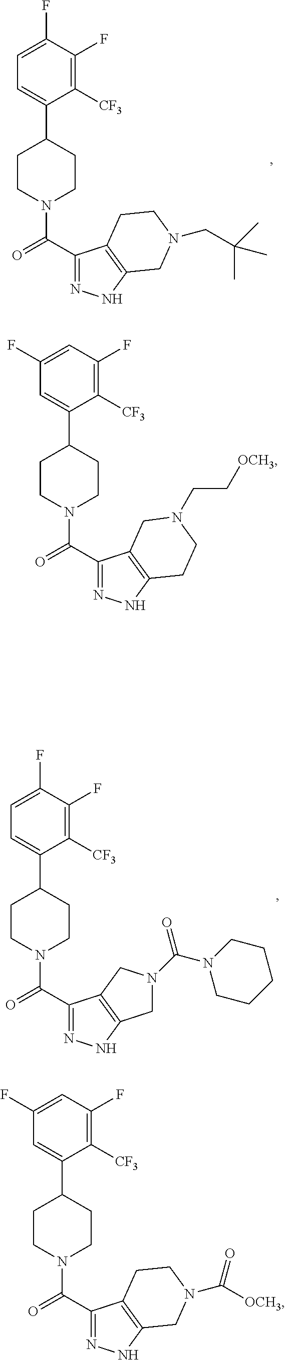 [SMILES: CC(C)(C)CN1CCc2c(C(=O)N3CCC(c4ccc(F)c(F)c4C(F)(F)F)CC3)n[nH]c2C1 ; COCCN1CCc2[nH]nc(C(=O)N3CCC(c4cc(F)cc(F)c4C(F)(F)F)CC3)c2C1 ; O=C(c1n[nH]c2c1CN(C(=O)N1CCCCC1)C2)N1CCC(c2ccc(F)c(F)c2C(F)(F)F)CC1 ; COC(=O)N1CCc2c(C(=O)N3CCC(c4cc(F)cc(F)c4C(F)(F)F)CC3)n[nH]c2C1]

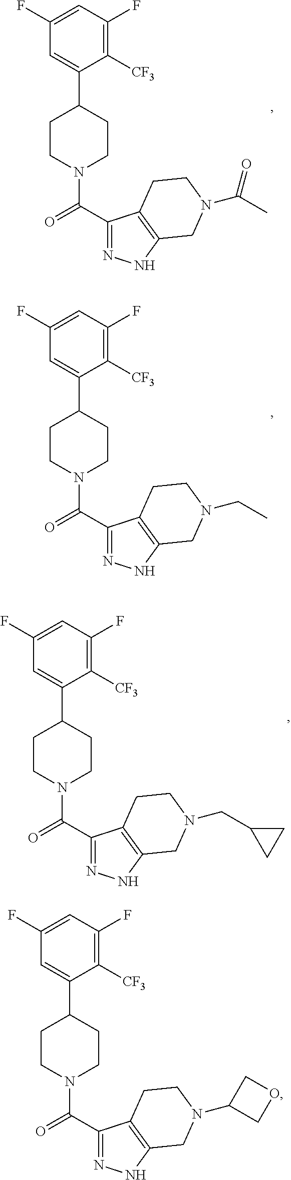

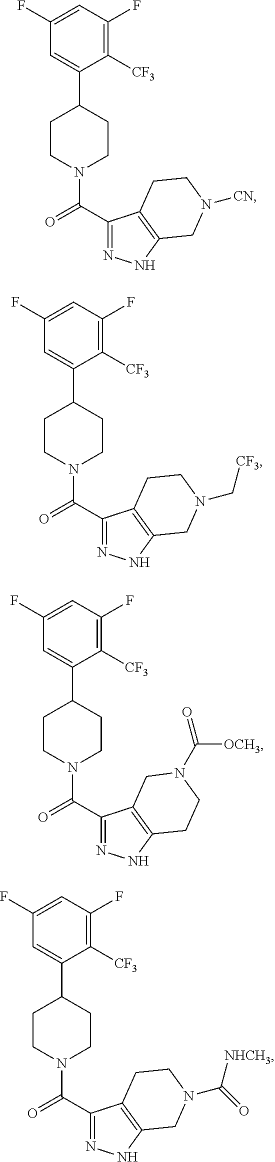

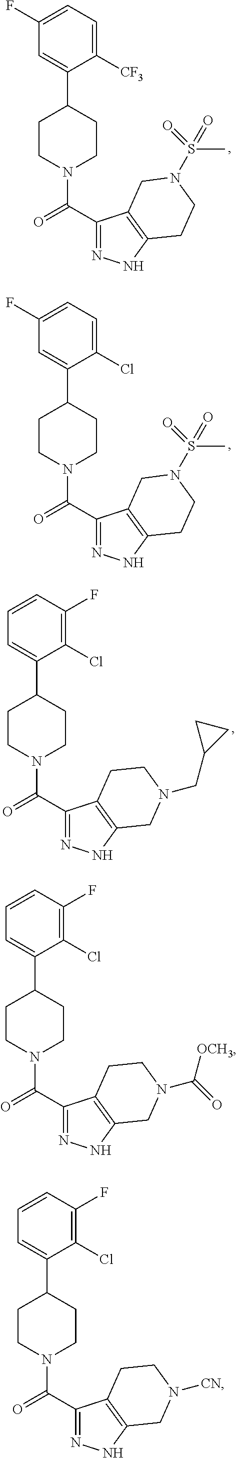

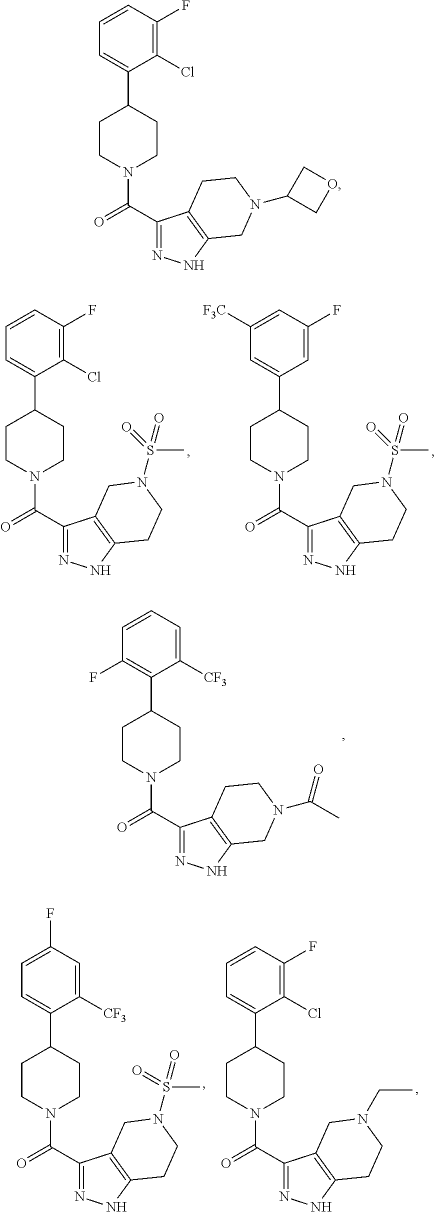

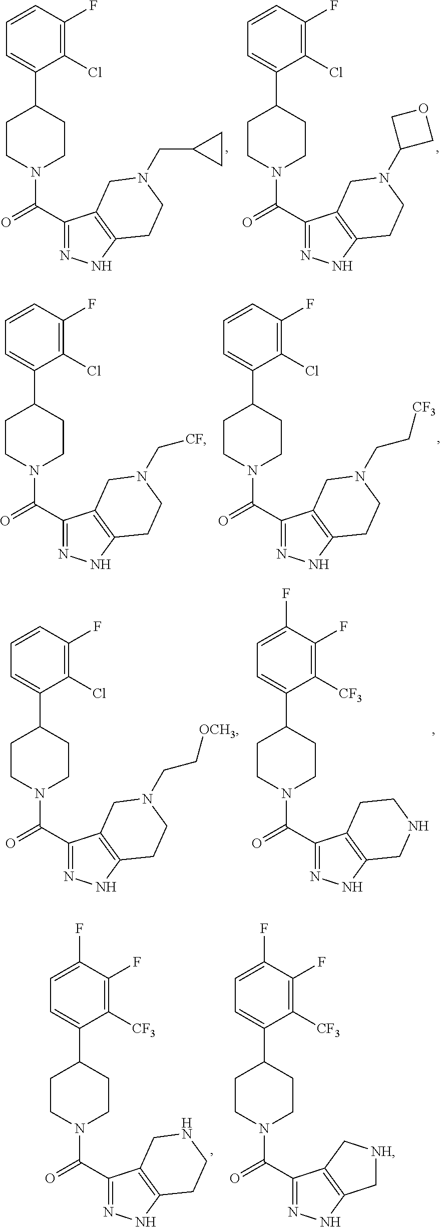

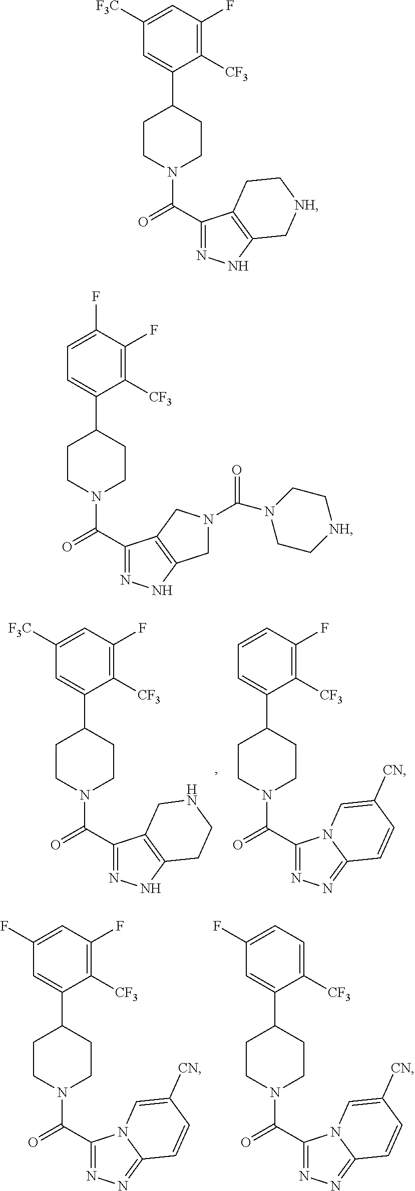

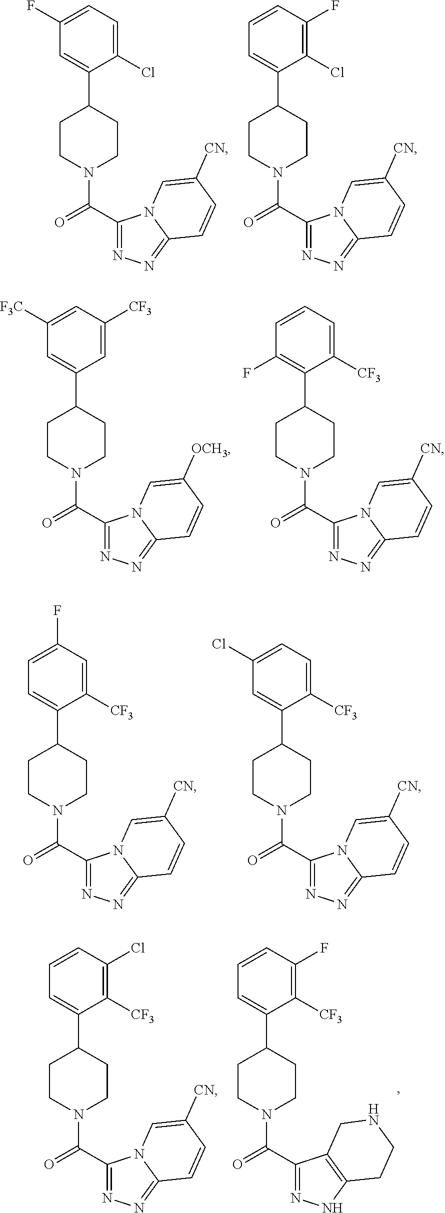

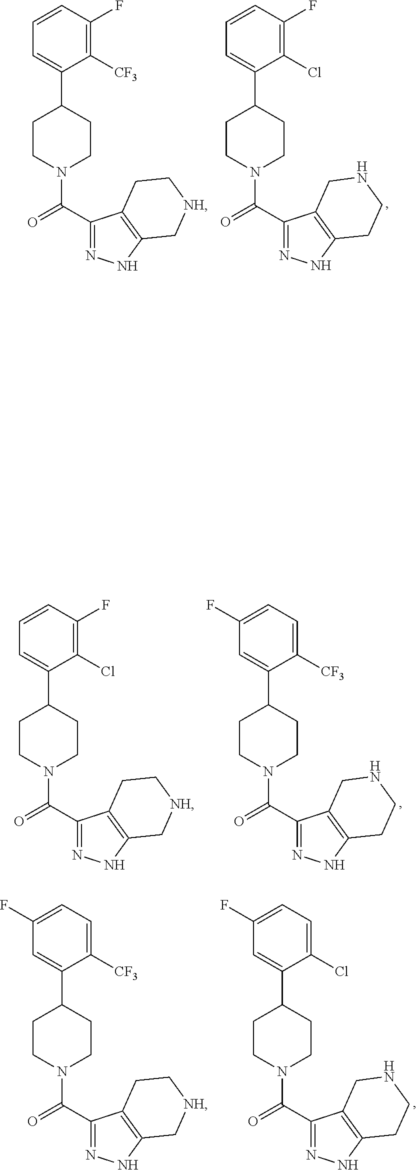

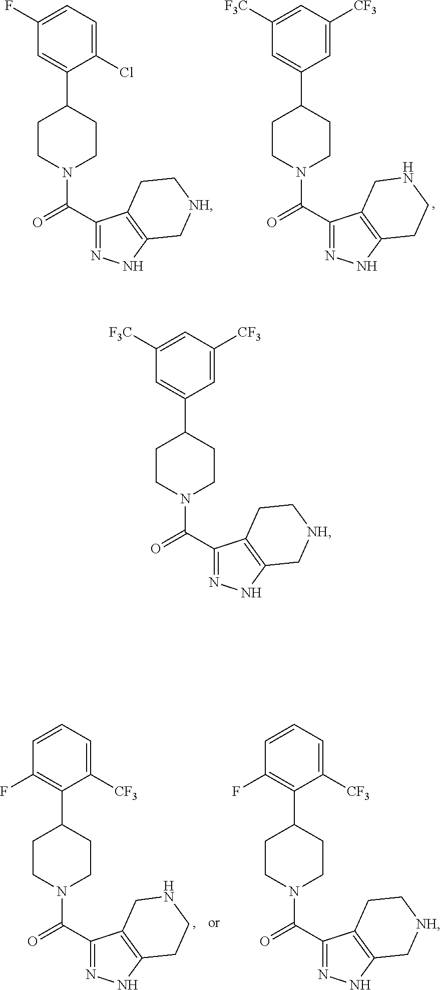 [SMILES: O=C(c1n[nH]c2c1CCNC2)N1CCC(c2cc(F)ccc2Cl)CC1 ; O=C(c1n[nH]c2c1CNCC2)N1CCC(c2cc(C(F)(F)F)cc(C(F)(F)F)c2)CC1 ; O=C(c1n[nH]c2c1CCNC2)N1CCC(c2cc(C(F)(F)F)cc(C(F)(F)F)c2)CC1 ; O=C(c1n[nH]c2c1CNCC2)N1CCC(c2c(F)cccc2C(F)(F)F)CC1 ; O=C(c1n[nH]c2c1CCNC2)N1CCC(c2c(F)cccc2C(F)(F)F)CC1]

or a pharmaceutically acceptable salt thereof.

In some embodiments of the method, the selective RBP4 antagonist is

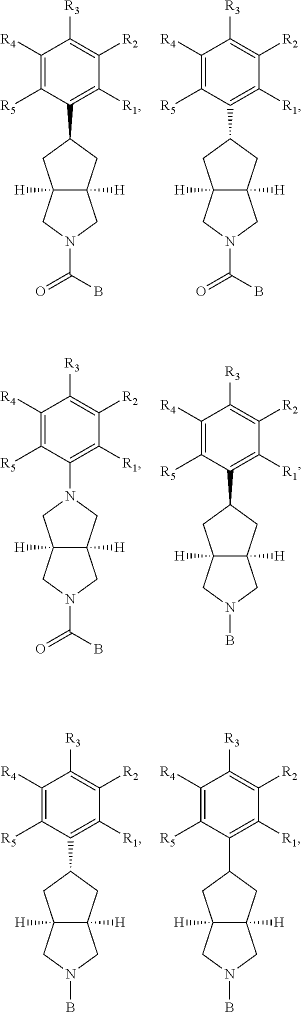

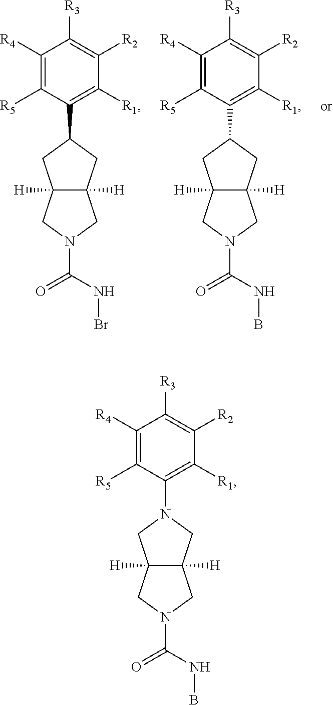

or a pharmaceutically acceptable salt thereof.

In some embodiments of the method, the selective RBP4 antagonist is

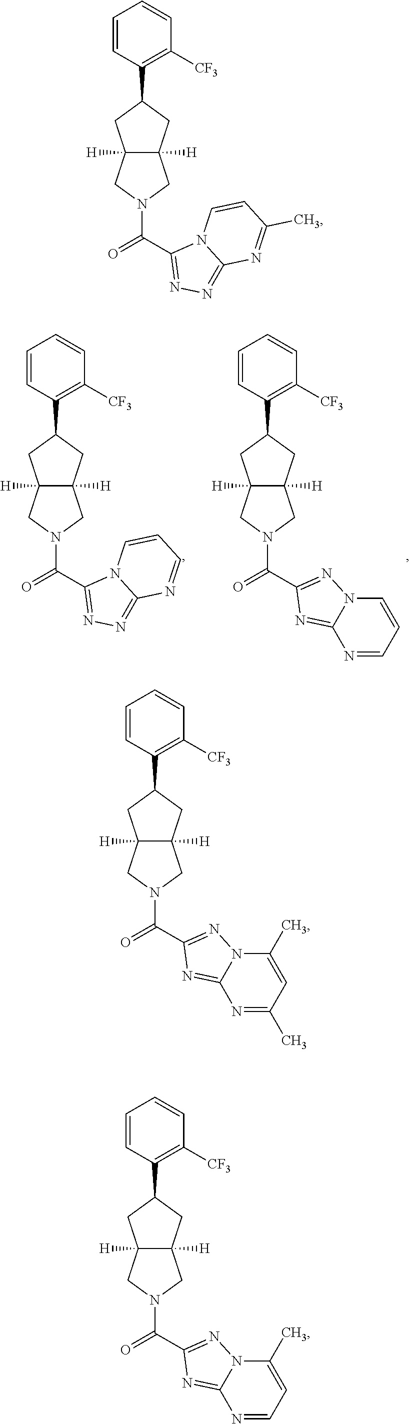 [SMILES: Cc1ccn2c(C(=O)N3C[C@H]4C[C@@H](c5ccccc5C(F)(F)F)C[C@H]4C3)nnc2n1 ; O=C(c1nnc2ncccn12)N1C[C@H]2C[C@@H](c3ccccc3C(F)(F)F)C[C@H]2C1 ; O=C(c1nc2ncccn2n1)N1C[C@H]2C[C@@H](c3ccccc3C(F)(F)F)C[C@H]2C1 ; Cc1cc(C)n2nc(C(=O)N3C[C@H]4C[C@@H](c5ccccc5C(F)(F)F)C[C@H]4C3)nc2n1 ; Cc1ccnc2nc(C(=O)N3C[C@H]4C[C@@H](c5ccccc5C(F)(F)F)C[C@H]4C3)nn12]

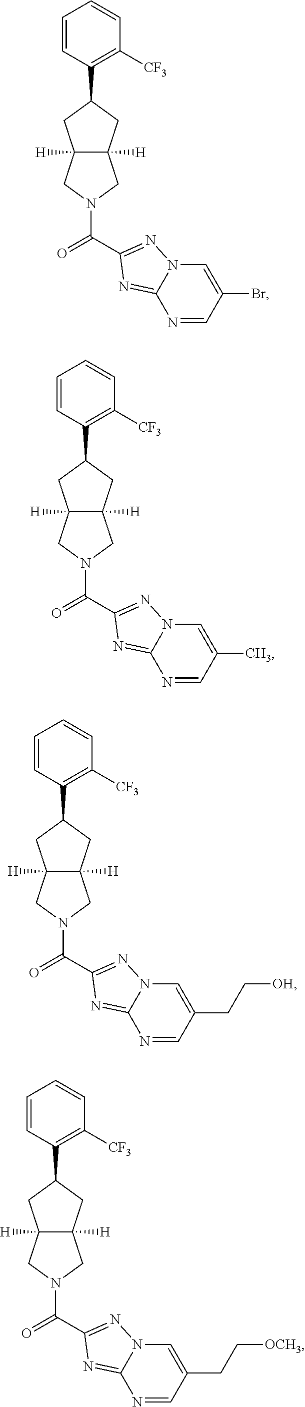 [SMILES: O=C(c1nc2ncc(Br)cn2n1)N1C[C@H]2C[C@@H](c3ccccc3C(F)(F)F)C[C@H]2C1 ; Cc1cnc2nc(C(=O)N3C[C@H]4C[C@@H](c5ccccc5C(F)(F)F)C[C@H]4C3)nn2c1 ; O=C(c1nc2ncc(CCO)cn2n1)N1C[C@H]2C[C@@H](c3ccccc3C(F)(F)F)C[C@H]2C1 ; COCCc1cnc2nc(C(=O)N3C[C@H]4C[C@@H](c5ccccc5C(F)(F)F)C[C@H]4C3)nn2c1]

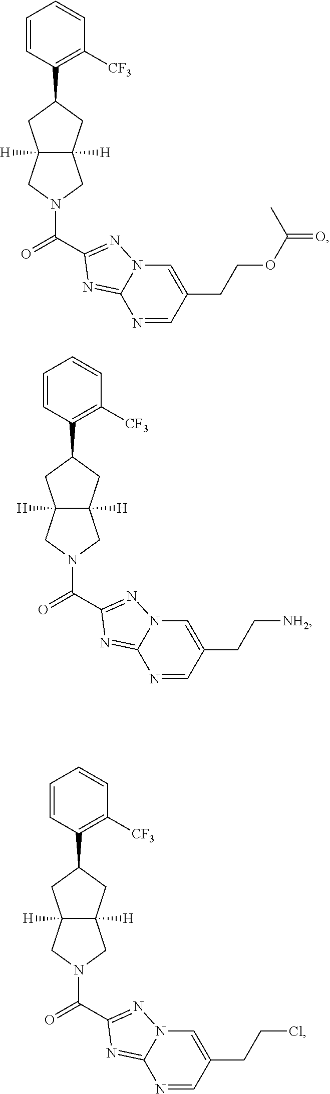 [SMILES: CC(=O)OCCc1cnc2nc(C(=O)N3C[C@H]4C[C@@H](c5ccccc5C(F)(F)F)C[C@H]4C3)nn2c1 ; NCCc1cnc2nc(C(=O)N3C[C@H]4C[C@@H](c5ccccc5C(F)(F)F)C[C@H]4C3)nn2c1 ; O=C(c1nc2ncc(CCCl)cn2n1)N1C[C@H]2C[C@@H](c3ccccc3C(F)(F)F)C[C@H]2C1]

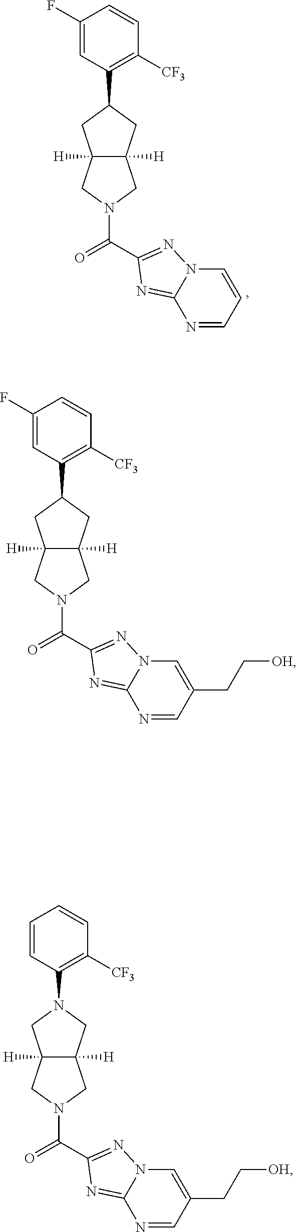

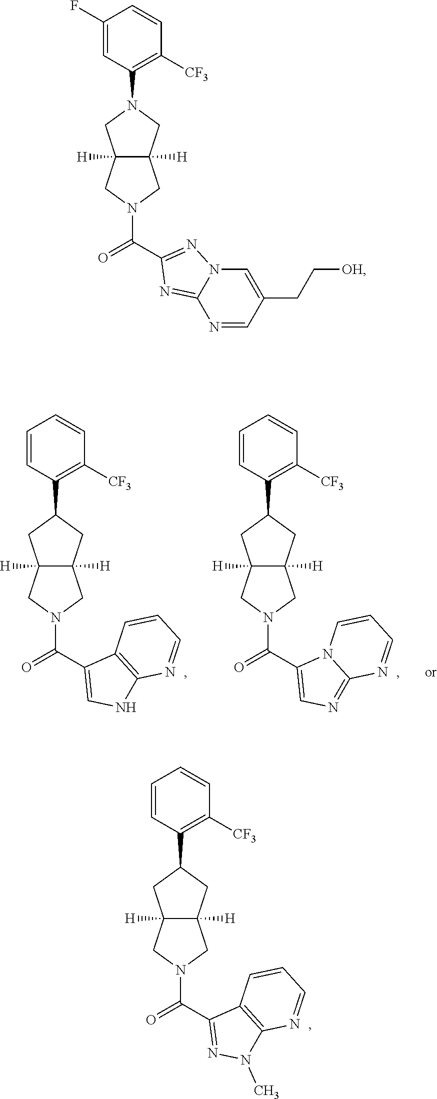

or a pharmaceutically acceptable salt thereof.

In some embodiments of the method, the selective RBP4 antagonist is the compound having the structure

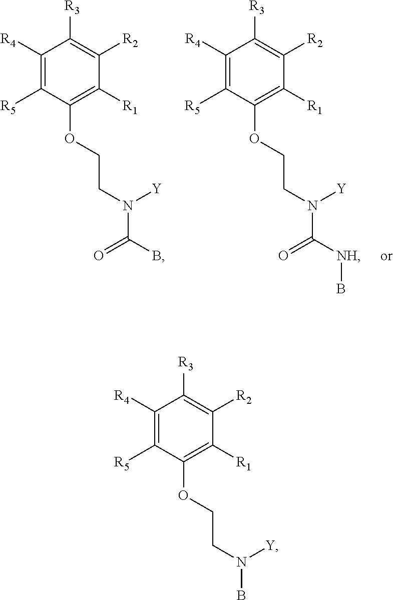

or a pharmaceutically acceptable salt thereof.

In some embodiments of the method, the selective RBP4 antagonist is

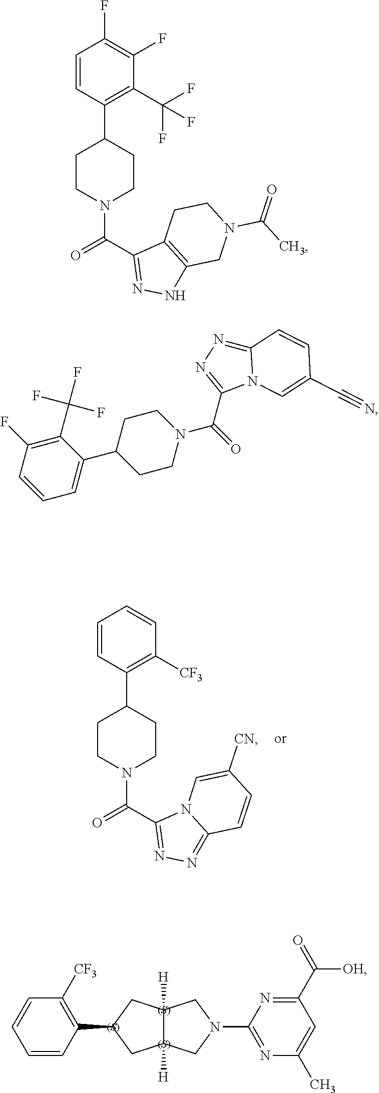

or a pharmaceutically acceptable salt thereof.

In some embodiments of the method, the selective RBP4 antagonist is the compound having the structure:

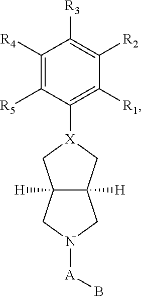

wherein
R₁, R₂, R₃, R₄, and R₅ are each independently H, halogen, CF₃, or C₁-C₄ alkyl;
X is N or CR₆,
   wherein R₆ is H, OH, or halogen;
A is absent or present, and when present, is

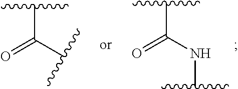

B has the structure:

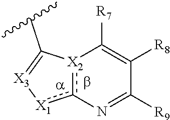

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

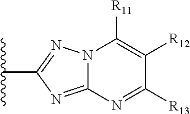

wherein
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments of the method, the selective RBP4 antagonist is the compound having the structure

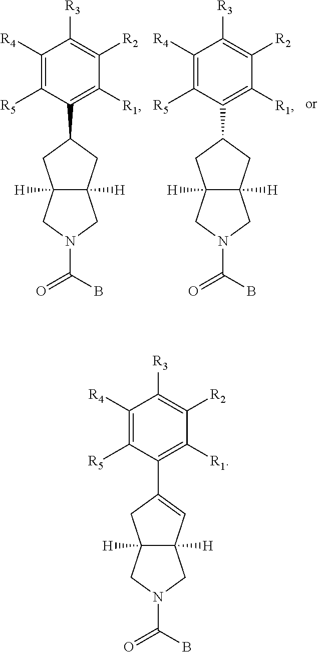

In some embodiments of the method, the selective RBP4 antagonist is the compound having the structure

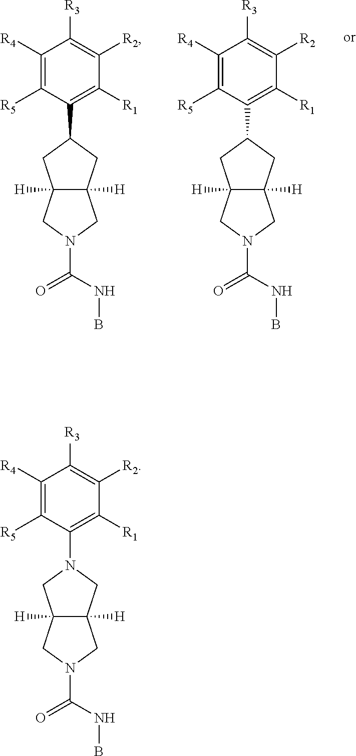

In some embodiments of the method, the selective RBP4 antagonist is the compound having the structure

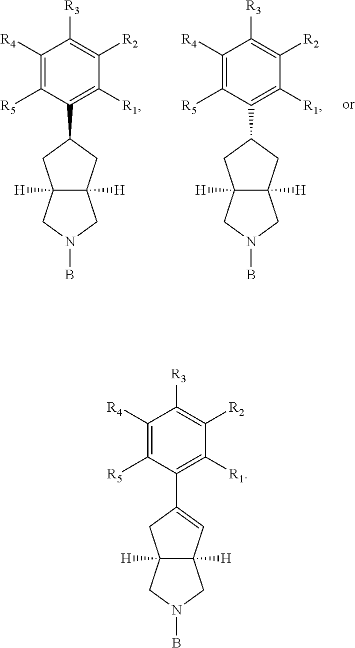

In some embodiments of the method, the selective RBP4 antagonist is the compound having the structure:

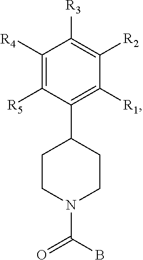

wherein
R₁, R₂, R₃, R₄, and R₅ are each independently H, halogen, CF₃, or C₁-C₄ alkyl; and
B has the structure:

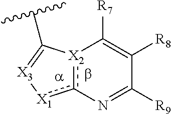

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

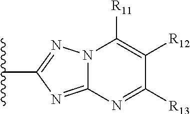

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
   or a pharmaceutically acceptable salt thereof.

In some embodiments of the method, the selective RBP4 antagonist is the compound having the structure:

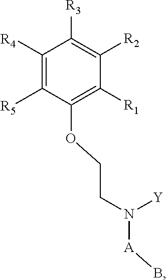

wherein
R₁, R₂, R₃, R₄, and R₅ are each independently H, halogen, CF₃, or C₁-C₄ alkyl;
Y is alkyl;
A is absent or present, and when present, is

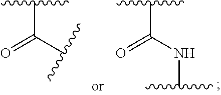

and
B has the structure:

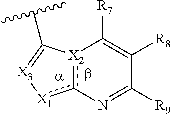

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

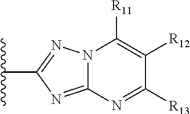

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
   or a pharmaceutically acceptable salt thereof.

In some embodiments of the method, the selective RBP4 antagonist is the compound having the structure

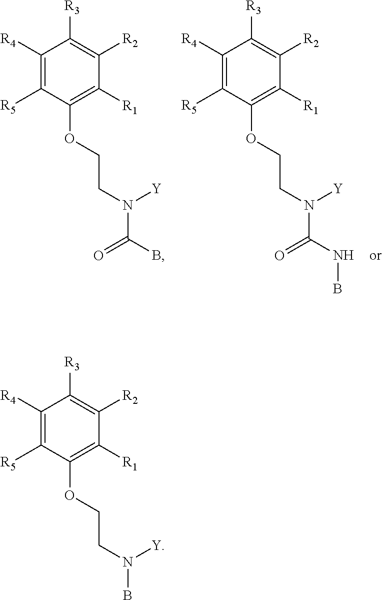

In some embodiments of the method, the selective RBP4 antagonist is the compound wherein B has the structure:

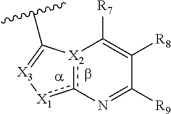

wherein
α and β are each a bond that is present or absent;
X₁ is N, NH, or NR₁₀,
   wherein R₁₀ is alkyl, alkenyl, or alkynyl;
X₂ is C or N;
X₃ is CH or N; and
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H.

In some embodiments of the method, the selective RBP4 antagonist is the compound wherein B or B₂ has the structure

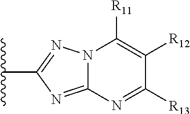

wherein
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments of the method, the selective RBP4 antagonist is the compound wherein B or B₂ has the structure

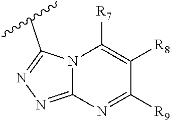

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments of the method, the selective RBP4 antagonist is the compound wherein B or B₂ has the structure

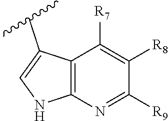

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments of the method, the selective RBP4 antagonist is the compound wherein B or B₂ has the structure

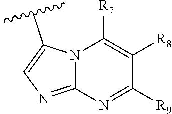

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments of the method, the selective RBP4 antagonist is the compound wherein B or B₂ has the structure:

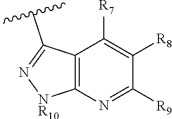

wherein
R₇, R₈, and R₉ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃; and
R₁₀ is alkyl, alkenyl, or alkynyl.

In some embodiments of the method, the selective RBP4 antagonist is the compound wherein R₇, R₈, and R₉ are each independently H, Cl, Br, F, OCH₃, OCH₂CH₃, CF₃, CN, CH₃, CH₂CH₃, C(O)OH, or C(O)—NH₂.

In some embodiments of the method, the selective RBP4 antagonist is the compound wherein R₇, R₈, and R₉ are each independently H, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein R₇, R₈, and R₉ are each independently H, halogen, or alkyl.

In some embodiments of the method, the selective RBP4 antagonist is the compound wherein two of R₇, R₈, and R₉ are each H and the remaining one of R₇, R₈, and R₉ is other than H.

In some embodiments of the method, the selective RBP4 antagonist is the compound wherein one of R₇, R₈, and R₉ is H and the remaining two of R₇, R₈, and R₉ are each other than H.

In some embodiments of the method, the selective RBP4 antagonist is the compound wherein B or B₂ has the structure

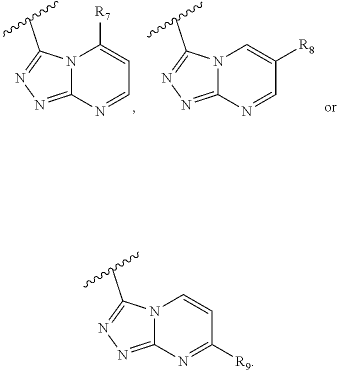

In some embodiments of the method, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein B or B₂ has the structure

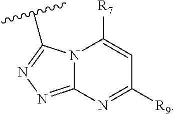

In some embodiments of the method, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein B or B₂ has the structure

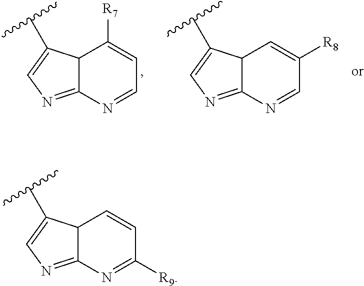

In some embodiments of the method, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein B or B₂ has the structure

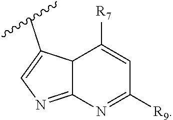

In some embodiments of the method, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein B has the structure

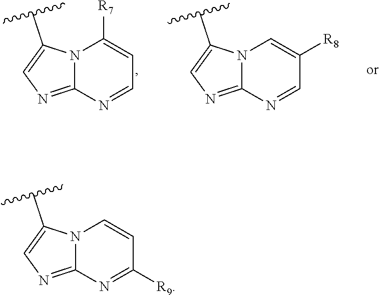

In some embodiments of the method, the selective RBP4 antagonist is the compound wherein R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein B or B₂ has the structure

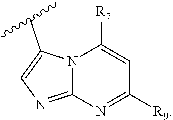

In some embodiments of the method, the selective RBP4 antagonist is the compound wherein R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein B or B₂ has the structure

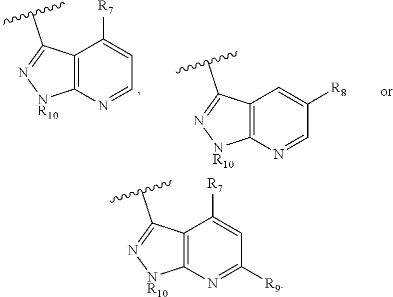

In some embodiments of the method, the selective RBP4 antagonist is the compound wherein:
R₇, R₈, and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br; and R₁₀ is alkyl.

In some embodiments of the method, the selective RBP4 antagonist is the compound wherein B or B₂ has the structure

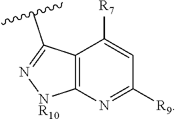

In some embodiments of the method, the selective RBP4 antagonist is the compound wherein:
R₇ and R₉ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br; and
R₁₀ is alkyl.

In some embodiments of the method, the selective RBP4 antagonist is the compound wherein B or B₂ has the structure

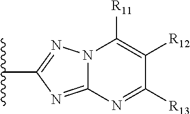

wherein R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃.

In some embodiments of the method, the selective RBP4 antagonist is the compound wherein R₁₁, R₁₂, and R₁₃ are each independently H, Cl, Br, F, OCH₃, OCH₂CH₃, CF₃, CN, CH₃, CH₂CH₃, C(O)OH, or C(O)—NH₂.

In some embodiments of the method, the selective RBP4 antagonist is the compound wherein R₁₁, R₁₂, and R₁₃ are each independently H, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein R₁₁, R₁₂, and R₁₃ are each independently H, halogen, or alkyl.

In some embodiments of the method, the selective RBP4 antagonist is the compound wherein two of R₁₁, R₁₂, and R₁₃ are each H and the remaining one of R₁₁, R₁₂, and R₁₃ is other than H.

In some embodiments of the method, the selective RBP4 antagonist is the compound wherein one of R₁₁, R₁₂, and R₁₃ is H and the remaining two of R₁₁, R₁₂, and R₁₃ are each other than H.

In some embodiments of the method, the selective RBP4 antagonist is the compound wherein B or B₂ has the structure

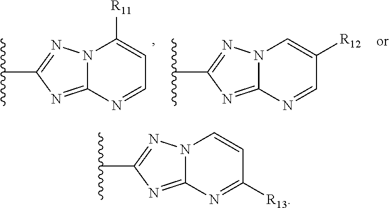

In some embodiments of the method, the selective RBP4 antagonist is the compound wherein R₁₁, R₁₂, and R₁₃ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein B or B₂ has the structure

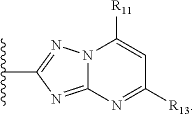

In some embodiments of the method, the selective RBP4 antagonist is the compound wherein R₁₁ and R₁₃ are each independently H, CH₃, Br, Cl, F, CH₂CH₂OH, CH₂CH₂OCH₃, CH₂CH₂OAc, CH₂CH₂Cl, CH₂CH₂F, or CH₂CH₂Br.

In some embodiments of the method, the selective RBP4 antagonist is the compound wherein X is N.

In some embodiments of the method, the selective RBP4 antagonist is the compound wherein X is CH.

In some embodiments of the method, the selective RBP4 antagonist is the compound wherein R₁, R₂, R₃, R₄, and R₅ are each H, t-Bu, Cl, F, or CF₃.

In some embodiments of the method, the selective RBP4 antagonist is the compound wherein:
R₁, R₂, R₃, and R₄ are each H; and
R₅ is CF₃ or t-Bu.

In some embodiments of the method, the selective RBP4 antagonist is the compound wherein:
R₁, R₃, and R₄ are each H;
R₂ is halogen; and
R₅ is CF₃ or t-Bu.

In some embodiments of the method, the selective RBP4 antagonist is the compound wherein:
R₁, R₂, R₃, and R₄ are each H; and
R₅ is CF₃ or t-Bu.

In some embodiments of the method, the selective RBP4 antagonist is the compound wherein:
R₁, R₂, R₃, and R₄ are each H; and
R₅ is CF₃.

In some embodiments of the method, the selective RBP4 antagonist is the compound wherein one of R₁, R₂, R₃, R₄, and R₅ is other than H.

In some embodiments of the method, the selective RBP4 antagonist is the compound wherein two of R₁, R₂, R₃, R₄, and R₅ are other than H.

In some embodiments of the method, the selective RBP4 antagonist is the compound wherein two or more of R₁, R₂, R₃, R₄, and R₅ are other than H.

In some embodiments of the method, the selective RBP4 antagonist is the compound wherein three of R₁, R₂, R₃, R₄, and R₅ are other than H.

In some embodiments of the method, the selective RBP4 antagonist is the compound wherein three or more of R₁, R₂, R₃, R₄, and R₅ are other than H.

In some embodiments of the method, the selective RBP4 antagonist is

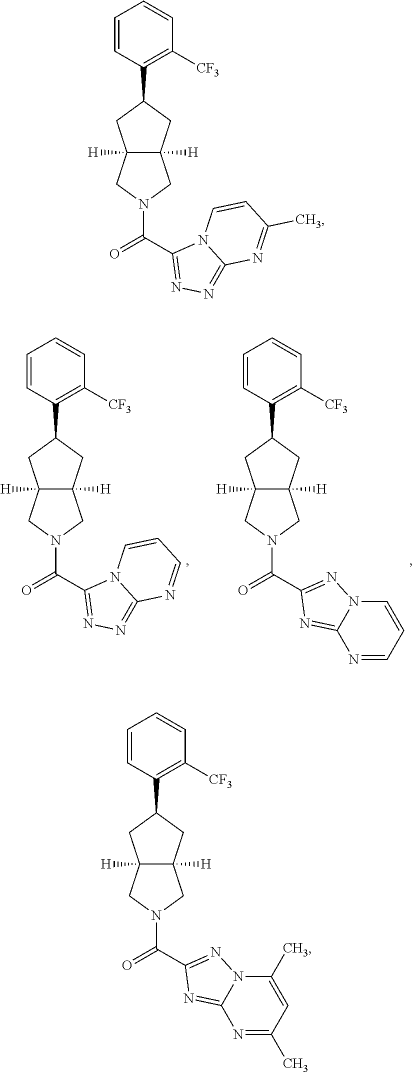 [SMILES: Cc1ccn2c(C(=O)N3C[C@H]4C[C@@H](c5ccccc5C(F)(F)F)C[C@H]4C3)nnc2n1 ; O=C(c1nnc2ncccn12)N1C[C@H]2C[C@@H](c3ccccc3C(F)(F)F)C[C@H]2C1 ; "," ; O=C(c1nc2ncccn2n1)N1C[C@H]2C[C@@H](c3ccccc3C(F)(F)F)C[C@H]2C1 ; "," ; Cc1cc(C)n2nc(C(=O)N3C[C@H]4C[C@@H](c5ccccc5C(F)(F)F)C[C@H]4C3)nc2n1]

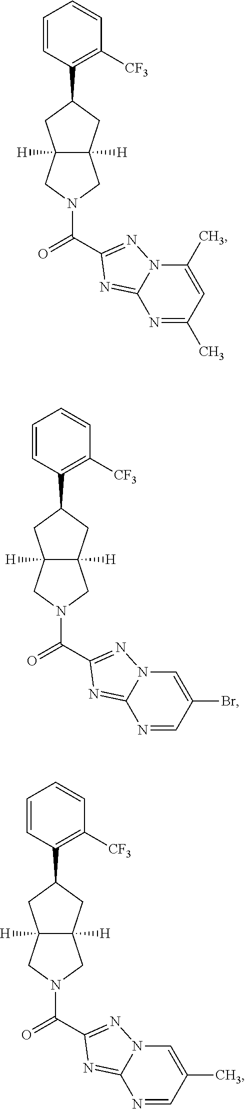 [SMILES: Cc1cc(C)n2nc(C(=O)N3C[C@H]4C[C@@H](c5ccccc5C(F)(F)F)C[C@H]4C3)nc2n1 ; O=C(c1nc2ncc(Br)cn2n1)N1C[C@H]2C[C@@H](c3ccccc3C(F)(F)F)C[C@H]2C1 ; Cc1cnc2nc(C(=O)N3C[C@H]4C[C@@H](c5ccccc5C(F)(F)F)C[C@H]4C3)nn2c1]

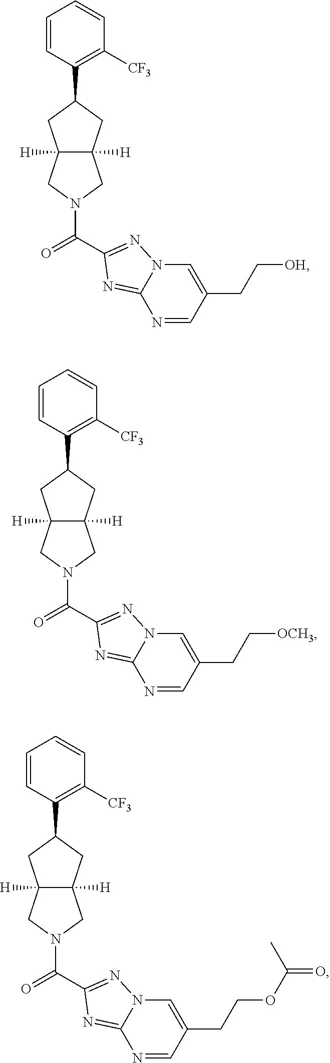 [SMILES: O=C(c1nc2ncc(CCO)cn2n1)N1C[C@H]2C[C@@H](c3ccccc3C(F)(F)F)C[C@H]2C1 ; COCCc1cnc2nc(C(=O)N3C[C@H]4C[C@@H](c5ccccc5C(F)(F)F)C[C@H]4C3)nn2c1 ; CC(=O)OCCc1cnc2nc(C(=O)N3C[C@H]4C[C@@H](c5ccccc5C(F)(F)F)C[C@H]4C3)nn2c1]

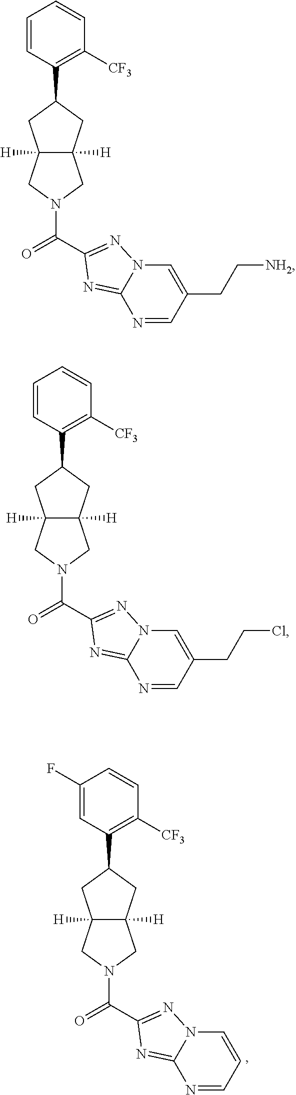 [SMILES: NCCc1cnc2nc(C(=O)N3C[C@H]4C[C@@H](c5ccccc5C(F)(F)F)C[C@H]4C3)nn2c1 ; O=C(c1nc2ncc(CCCl)cn2n1)N1C[C@H]2C[C@@H](c3ccccc3C(F)(F)F)C[C@H]2C1 ; O=C(c1nc2ncccn2n1)N1C[C@H]2C[C@@H](c3cc(F)ccc3C(F)(F)F)C[C@H]2C1]

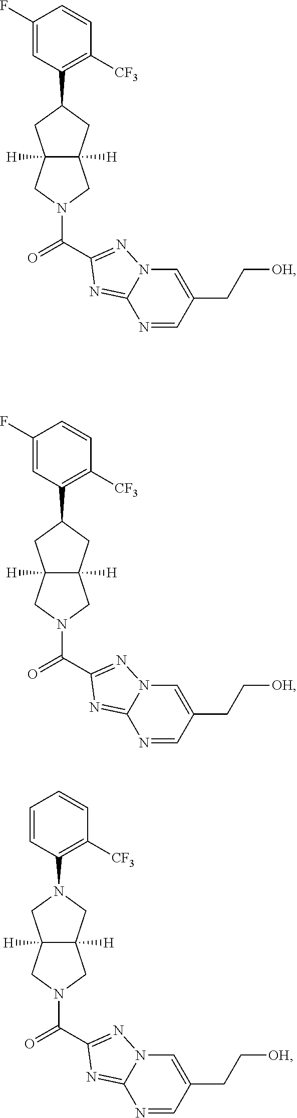

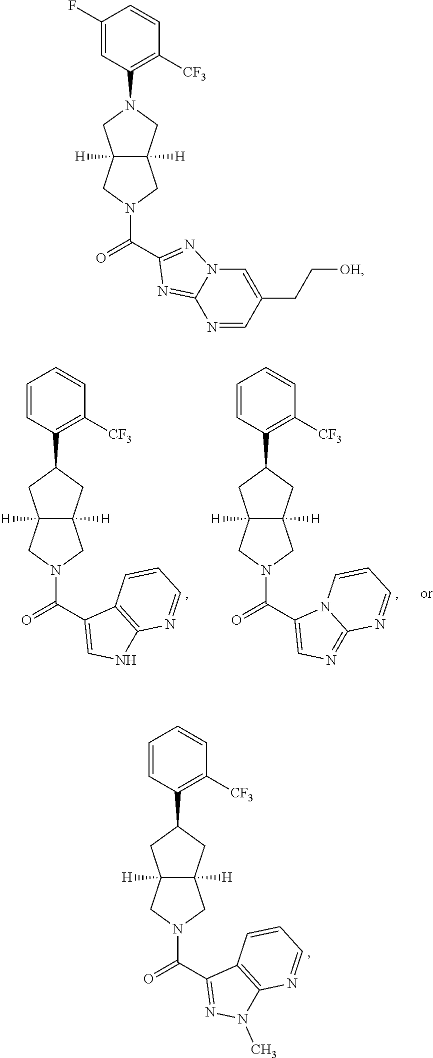

or a pharmaceutically acceptable salt thereof.

In some embodiments of the method, the selective RBP4 antagonist is the compound wherein B or B₂ is other than

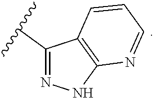 [SMILES: *c1n[nH]c2ncccc12]

In some embodiments of the method, the selective RBP4 antagonist is the compound having the structure:

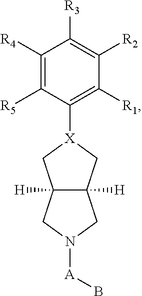

wherein
R₁, R₂, R₃, R₄, and R₅ are each independently H, halogen, alkyl, haloalkyl, O-haloalkyl, aryl, or heteroaryl;
X is N or CR₆,
   wherein R₆ is H, OH, or halogen;
A is absent or present, and when present, is

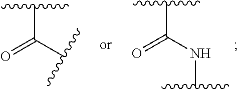

and
B has the structure:

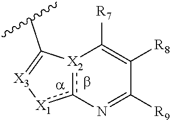

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

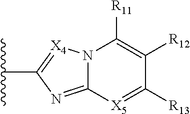

wherein
X₄ and X₅ are each independently N or CH; and
R₁₁, R₁₂, and R₁₃ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments of the method, the selective RBP4 antagonist is the compound having the structure:

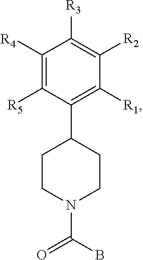

wherein
R₁, R₂, R₃, R₄, and R₅ are each independently H, halogen, CF₃, C₁-C₄ alkyl, aryl, or heteroaryl; and
B has the structure:

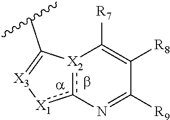

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

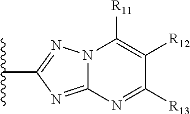

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments of the method, the selective RBP4 antagonist is the compound having the structure:

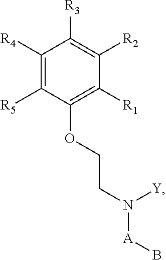

wherein
R₁, R₂, R₃, R₄, and R₅ are each independently H, halogen, CF₃, C₁-C₄ alkyl, aryl, or heteroaryl;
Y is alkyl;
A is absent or present, and when present, is

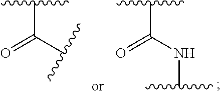

and
B has the structure:

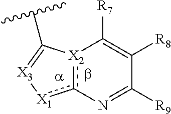

wherein
α and β are each a bond that is present or absent;
X₁ is N, NH, or NR₁₀,
   wherein R₁₀ is alkyl, alkenyl, or alkynyl;
X₂ is C or N;
X₃ is CH or N;
R₇, R₈, and R₉ are each independently H, halogen, alkyl, alkenyl, alkynyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃,
wherein
   X₁, X₂, and X₃ are each N, α is present, and β is absent; or
   X₁ is NH, X₂ is C, X₃ is CH, α is absent, and β is present; or
   X₁ is N, X₂ is N, X₃ is CH, α is present, and β is absent; or
   X₁ is NH or NR₁₀, X₂ is C, X₃ is N, α is absent, and β is present, wherein when X₁ is NH, X₂ is C, X₃ is N, α is absent, and β is present, then one of R₇, R₈, and R₉ is other than H;
or
B has the structure:

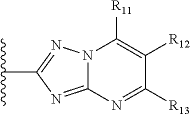

wherein
R₁₁, R₁₂, and R₁₃ are each independently H, halogen, alkyl, alkylenyl-OH, alkylenyl-NH₂, alkylenyl-OAc, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
   or a pharmaceutically acceptable salt thereof.

In some embodiments of the method, the selective RBP4 antagonist is the compound wherein one of R₁, R₂, R₃, R₄, and R₅ is other than H.

In some embodiments of the method, the selective RBP4 antagonist is the compound wherein two of R₁, R₂, R₃, R₄, and R₅ are other than H.

In some embodiments of the method, the selective RBP4 antagonist is the compound wherein R₁, R₂, R₃, R₄, and R₅ are each H, methyl, ethyl, phenyl, t-Bu, i-Pr, OCF₃, CF₃, OCF₂CF₃, CF₂CF₃, Cl, Br, or F.

In some embodiments of the method, the selective RBP4 antagonist is the compound wherein:
R₁, R₂, R₃, and R₄ are each H; and
R₅ is —H, OCF₃, CF₂CF₃, methyl, ethyl, i-Pr, or phenyl.

In some embodiments of the method, the selective RPB4 antagonist is

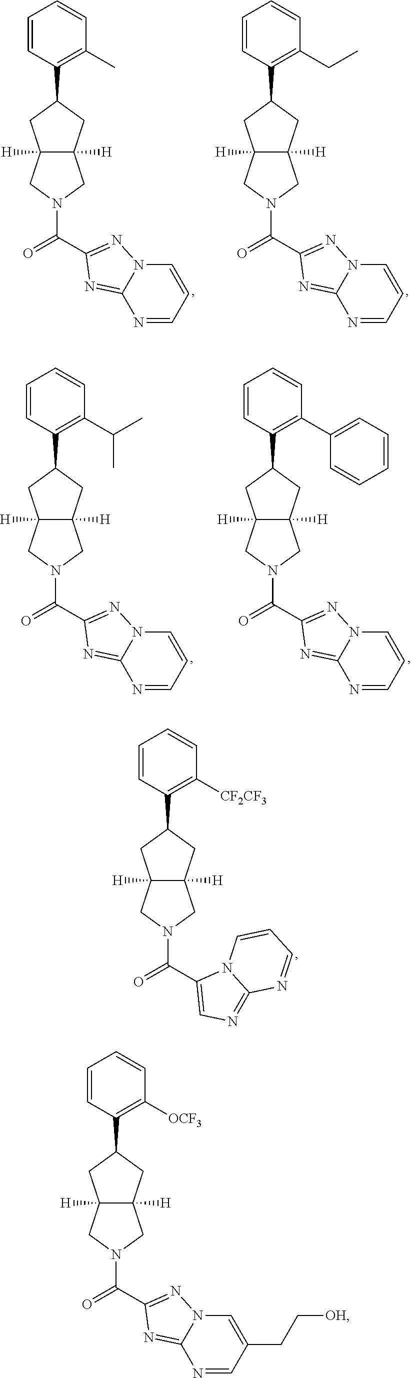

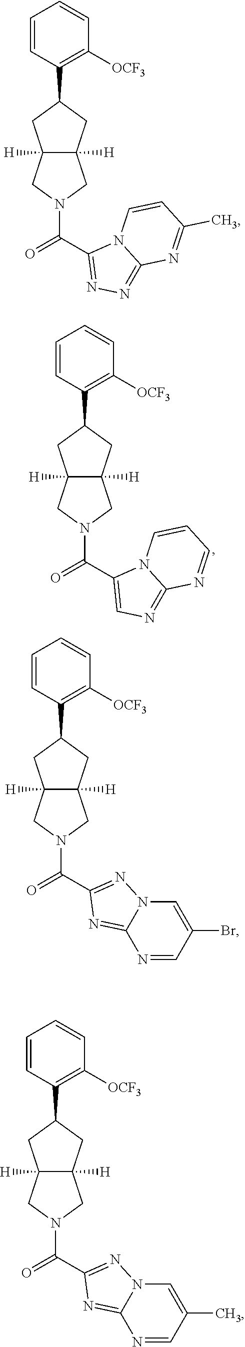

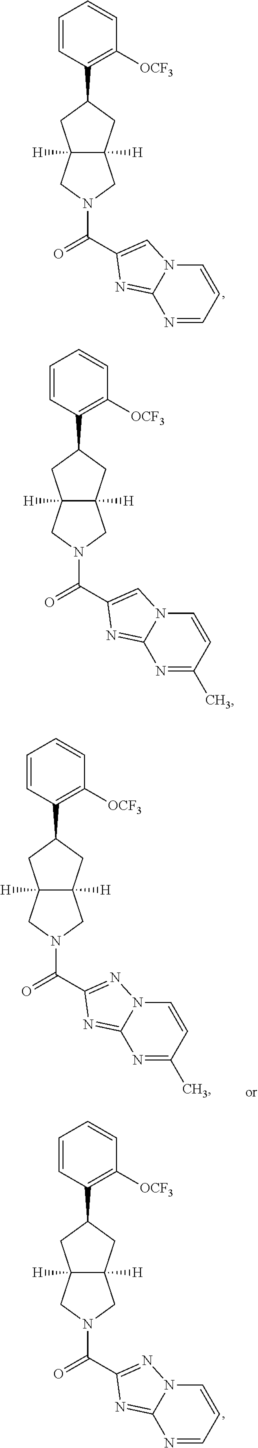 [SMILES: O=C(c1cn2cccnc2n1)N1C[C@H]2C[C@@H](c3ccccc3OC(F)(F)F)C[C@H]2C1 ; Cc1ccn2cc(C(=O)N3C[C@H]4C[C@@H](c5ccccc5OC(F)(F)F)C[C@H]4C3)nc2n1 ; Cc1ccn2nc(C(=O)N3C[C@H]4C[C@@H](c5ccccc5OC(F)(F)F)C[C@H]4C3)nc2n1 ; O=C(c1nc2ncccn2n1)N1C[C@H]2C[C@@H](c3ccccc3OC(F)(F)F)C[C@H]2C1]

or a pharmaceutically acceptable salt thereof.

Except where otherwise specified, if the structure of a compound of the present disclosure includes an asymmetric carbon atom, it is understood that the compound occurs as a racemate, a racemic mixture, scalemic mixtures, and isolated single enantiomers. All such isomeric forms of these compounds are expressly included in the present disclosure. Except where otherwise specified, each stereogenic carbon may be of the R or S configuration. It is to be understood accordingly that the isomers arising from such asymmetry (e.g., all enantiomers and diastereomers) are included within the scope of the present disclosure, unless indicated otherwise. Such isomers can be obtained in substantially pure form by classical separation techniques and by stereochemically controlled syntheses, such as those described in: “Enantiomers, Racemates and Resolutions” by J. Jacques, A. Collet and S. Wilen, Pub. John Wiley & Sons, NY, 1981. For example, the resolution may be carried out by preparative chromatography on a chiral column.

Except where otherwise specified, the present disclosure is intended to include all isotopes of atoms occurring on the compounds disclosed herein. Isotopes include those atoms having the same atomic number but different mass numbers. By way of a general example and without limitation, isotopes of hydrogen include tritium and deuterium. Isotopes of carbon include C-13 and C-14.

It will be noted that any notations of a carbon in structures throughout the present disclosure, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notations of a hydrogen (H) in structures throughout the present disclosure, when used without further notation, are intended to represent all isotopes of hydrogen, such as ¹H, ²H (D), or ³H (T) except where otherwise specified.

Furthermore, any compounds containing ²H or ³H may specifically have the structure of any of the compounds disclosed herein except where otherwise specified.

Isotopically labeled compounds can generally be prepared by conventional techniques known to those skilled in the art using appropriate isotopically-labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes ¹H (hydrogen or protium), D (2H or deuterium), and T (³H or tritium). The natural abundance of deuterium is 0.0156%. Thus, in a composition comprising molecules of a naturally occurring compound, the level of deuterium at a particular hydrogen atom site in that compound is expected to be 0.0156%. Thus, a composition comprising a compound with a level of deuterium at any site of hydrogen atom in the compound that has been enriched to be greater than its natural abundance of 0.0156% is novel over its naturally occurring counterpart. A person skilled in the art may use the techniques disclosed herein to prepare deuterium analogs thereof.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon or hydrogen atom(s) are replaced by one or more bonds, including double or triple bonds, to a heteroatom. Examples of substituent groups include: the functional groups described above; halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy groups, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro; nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds used in the method of the present disclosure, the substituents may be substituted or unsubstituted, unless specifically defined otherwise.

In the compounds used in the method of the present disclosure, alkyl, haloalkyl, alkenyl, alkynyl, aryl, heteroaryl, cycloalkyl, cycloalkylalkyl, heteroalkyl, heterocycle, heterocycloalkyl, alkylheteroalkyl, alkylaryl, monocycle, bicycle, heteromonocycle, and heterobicycle groups can be further substituted by replacing one or more hydrogen atoms with alternative non-hydrogen groups. These include, but are not limited to, halo, hydroxy, mercapto, amino, carboxy, cyano, and carbamoyl.

It is understood that substituents and substitution patterns on the compounds used in the method of the present disclosure can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present disclosure, one of ordinary skill in the art will recognize that the various substituents, i.e., R₁, R₂, etc., are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆ as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, and hexyl. Unless otherwise specified, an alkyl group contains one to ten carbons. Alkyl groups can be unsubstituted or substituted with one or more substituents, including but not limited to halogen, alkoxy, alkylthio, trifluoromethyl, difluoromethyl, methoxy, and hydroxyl. “Haloalkyl” includes any alkyl group containing at least one halogen atom.

The term “alkenyl” refers to a non-aromatic hydrocarbon radical, straight or branched, containing at least 1 carbon-to-carbon double bond, and up to the maximum possible number of non-aromatic carbon-carbon double bonds may be present. Thus, C₂-Cₙ alkenyl is defined to include groups having 2, 3 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl, and cyclohexenyl. As described above with respect to alkyl, the straight, branched, or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical, straight or branched, containing at least 1 carbon-to-carbon triple bond, and up to the maximum possible number of non-aromatic carbon-carbon triple bonds may be present. Thus, C₂-Cₙ alkynyl is defined to include groups having 2, 3 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl, and butynyl.

As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

The term “alkylenyl” refers to a divalent analog of a linear or branched alkyl group.

As used herein, “aryl” is intended to mean any stable monocyclic, bicyclic, or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl, or acenaphthyl. In cases where the aryl substituent is bicyclic and one ring is non-aromatic, it is understood that attachment is via the aromatic ring.

The term “heteroaryl”, as used herein, represents a stable monocyclic, bicyclic, or polycyclic ring of up to 10 atoms in each ring, wherein at least one ring is aromatic and contains from 1 to 4 heteroatoms selected from the group consisting of O, N, and S. Bicyclic aromatic heteroaryl groups include phenyl, pyridine, pyrimidine, or pyridazine rings that are: (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N, or S.

Heteroaryl groups within the scope of this definition include but are not limited to: benzimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, and tetra-hydroquinoline. In cases where the heteroaryl substituent is bicyclic and one ring is non-aromatic or contains no heteroatoms, it is understood that attachment is via the aromatic ring or via the heteroatom-containing ring, respectively. If the heteroaryl contains nitrogen atoms, it is understood that the corresponding N-oxides thereof are also encompassed by this definition.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl, or cyclooctyl). “Cycloalkylalkyl” includes any alkyl group containing at least one cycloalkyl ring.

As used herein, “C₁-C₄ alkyl” includes both branched and straight-chain C₁-C₄ alkyl.

As used herein, “heteroalkyl” includes both branched and straight-chain saturated aliphatic hydrocarbon groups having at least 1 heteroatom within the chain or branch. “Alkylheteroalkyl” includes any alkyl group containing at least one heteroalkyl group.

The term “heterocycle”, “heterocyclyl”, or “heterocyclic” refers to a mono- or poly-cyclic ring system which can be saturated or contains one or more degrees of unsaturation, and contains one or more heteroatoms. Preferred heteroatoms include N, O, and/or S, including N-oxides, sulfur oxides, and dioxides. Preferably the ring is three to ten-membered, and is either saturated or has one or more degrees of unsaturation. The heterocycle may be unsubstituted or substituted, with multiple degrees of substitution being allowed. Such rings may be optionally fused to one or more of another heterocyclic ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

As used herein, “heterocyclyl” is intended to mean a 5- to 10-membered nonaromatic ring containing from 1 to 4 heteroatoms selected from the group consisting of O, N, and S, and includes bicyclic groups. “Heterocyclyl” therefore includes but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl, and the like. If the heterocycle contains nitrogen, it is understood that the corresponding N-oxides thereof are also encompassed by this definition.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of alkylaryl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl, and the like.

As used herein, “monocycle” includes any stable cyclic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “heteromonocycle” includes any monocycle containing at least one heteroatom.

As used herein, “bicycle” includes any stable cyclic carbon ring of up to 10 atoms that is fused to a cyclic carbon ring of up to 10 atoms with each ring being independently unsubstituted or substituted.

Examples of such non-aromatic bicycle elements include but are not limited to: decahydronaphthalene. Examples of such aromatic bicycle elements include but are not limited to: naphthalene. As used herein, “heterobicycle” includes any bicycle containing at least one heteroatom.

The compounds used in the method of the present disclosure may be prepared by techniques well known in organic syntheses and familiar to a practitioner ordinarily skilled in the art. However, these may not be the only means by which to synthesize or obtain the desired compounds.

The compounds used in the method of the present disclosure may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example, those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the present disclosure comprises a compound or composition of the present disclosure as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present disclosure using standard esterification reactions and methods readily available and known to those having ordinary skill in the art of chemical syntheses. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present disclosure may be in a salt form. As used herein, a “salt” is a salt of the instant compounds which has been modified by making acid or base salts of the compounds. In the case of compounds used to treat a disease or medical disorder, the salt is pharmaceutically acceptable. Examples of pharmaceutically acceptable salts include, but are not limited to: mineral or organic acid salts of basic residues such as amines; alkali or organic salts of acidic residues such as phenols; and alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the sodium, potassium, or lithium salts, and the like. Carboxylate salts are the sodium, potassium, or lithium salts, and the like. The term “pharmaceutically acceptable salt” in this respect refers to the relatively non-toxic, inorganic or organic, acid or base addition salts of compounds of the present disclosure. These salts can be prepared in situ during the final isolation and purification of the compounds of the present disclosure, or by separately reacting a purified compound of the present disclosure in its free base or free acid form with a suitable organic or inorganic acid or base, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. (See, e.g., Berge et al. (1977) “Pharmaceutical Salts”, J. Pharm. Sci. 66:1-19).

A salt or pharmaceutically acceptable salt is contemplated for all compounds disclosed herein.

As used herein, “treating” means preventing, slowing, halting, or reversing the progression of a disease. Treating may also mean improving one or more symptoms of a disease.

As used herein, “normalize,” as to normalize a concentration in a subject afflicted with a disease, means increasing or reducing the concentration such that the concentration is closer to what the concentration would be in a subject without the disease.

As used herein, “elevated,” as in the RBP4 concentration is elevated in a subject afflicted with a disease, means that the RBP4 concentration is elevated in comparison to what the concentration would be in a subject without the disease.

The compounds used in the method of the present disclosure may be administered in various forms, including those detailed herein. The treatment with the compound may be a component of a combination therapy or an adjunct therapy, i.e., the subject or patient in need of the drug is treated with or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent, or vehicle, for delivering the instant compounds to the animal or human. The carrier may be liquid or solid and is selected with the planned manner of administration in mind. Liposomes are also a pharmaceutically acceptable carrier, as are capsules, coatings, and various syringes.

The dosage of the compounds administered in treatment will vary depending upon factors such as: the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment; and the desired therapeutic effect.

A dosage unit of the compounds used in the method of the present disclosure may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g., by injection, topical application, or other methods, into or onto a site of disease, all using dosage forms well known to those of ordinary skill in the pharmaceutical arts.

The compounds used in the method of the present disclosure can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous, or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder, or in a liquid form. Examples of suitable solid carriers include lactose, sucrose, gelatin, and agar. Capsule or tablets can be easily formulated and can be made easy to swallow or chew. Other solid forms include granules and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups, or elixirs, suspensions, solutions and/or suspensions reconstituted from non-effervescent granules and effervescent preparations reconstituted from effervescent granules.

Such liquid dosage forms may contain, for example, suitable solvents, preservatives, emulsifying agents, suspending agents, diluents, sweeteners, thickeners, and melting agents. Oral dosage forms optionally contain flavoring and coloring agents. Parenteral and intravenous forms may also include minerals and other materials to make them compatible with the type of injection or delivery system chosen.

Techniques and compositions for making dosage forms useful in the present disclosure are described in the following references: 7 Modern Pharmaceutics, Chapters 9 and 10 (Banker & Rhodes, Editors, 1979); Pharmaceutical Dosage Forms: Tablets (Lieberman et al. 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); and Modern Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, 17th ed., 1989, a standard reference text in this field.

The compounds used in the method of the present disclosure may also be administered in intranasal form via use of suitable intranasal vehicles, or via transdermal routes, using those forms of transdermal skin patches well known to those of ordinary skill in the art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the present disclosure. Any of the disclosed generic or specific compounds may be applicable to any of the disclosed compositions, processes, or methods.

### Experimental Details

### Co-Administration of the Selective TTR Ligand and β-Carotene Restored the Concentration of Functional Rhodopsin in Mice.

ACPHS-14 was previously identified and described as a selective TTR ligand capable of significantly decreasing the concentration of serum RBP4 in mice while inducing stabilization of TTR tetramers (Cioffi, C. L. et al. 2021). Thus, ACPHS-14 was used as a representative “first component” of the present disclosure in order to assess the feasibility of using β-carotene as the “second component” in the co-drug strategy. Accordingly, an experiment was conducted to determine whether partial reduction in functional rhodopsin (opsin with conjugated 11-cis-retinal) induced by oral ACPHS-14 in mice can be compensated by co-administration with β-carotene. An experiment was also conducted to determine whether co-administration of ACPHS-14 and β-carotene can restore retinal function as can be assessed by electroretinography (ERG). It was previously reported that ACPHS-14 can induce a pronounced reduction in serum RBP4 along with stabilization of TTR tetramers (Cioffi, C. L. et al. 2021).

The experiments were conducted in Balb/c mice. Three groups of mice were used: control untreated mice; mice treated with ACPHS-14; and mice treated with the ACPHS-14 plus β-carotene combination. ACPHS-14 was administered through oral gavage at a 25 mg/kg dose formulated into the chow to ensure a standard daily oral dosing of the compound at the 25 mg/kg dose. β-carotene was dissolved in olive oil and was administered through oral gavage daily at a daily 6 mg/kg dose. Dosing duration was 10 days. Following compound dosing, retinal function in the mice was assessed by ERG as previously described (Racz, B. et al. 2018). Following ERG analysis, retinal extracts were prepared as previously described (Racz, B. et al. 2018). Rhodopsin was measured spectrophotometrically in the retinal extracts using a previously published protocol (Racz, B. et al. 2018).

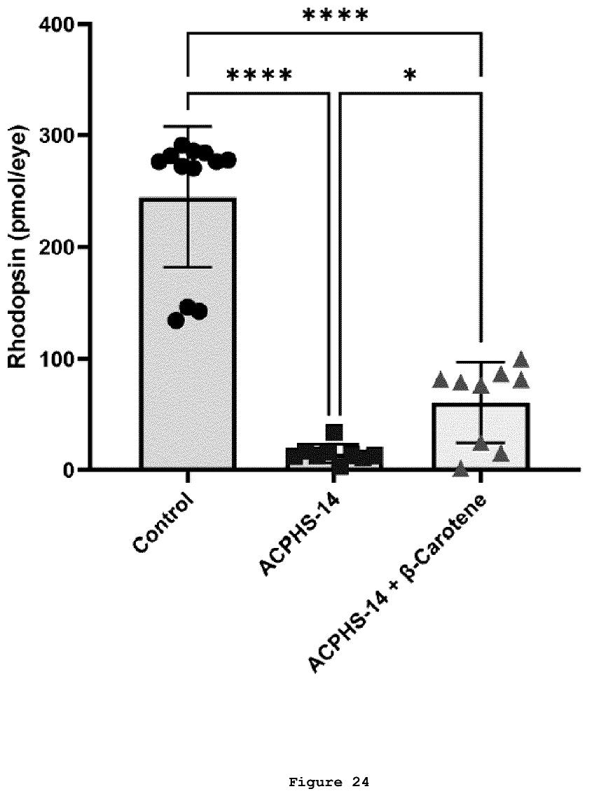

As shown in FIG. 24, ACPHS-14 given for 10 days to wild-type mice significantly reduced rhodopsin in comparison to untreated mice. On the other hand, co-administration of ACPHS-14 with β-carotene induced a 4-fold increase in concentration of rhodopsin in comparison to the ACPHS-14-treated mice.

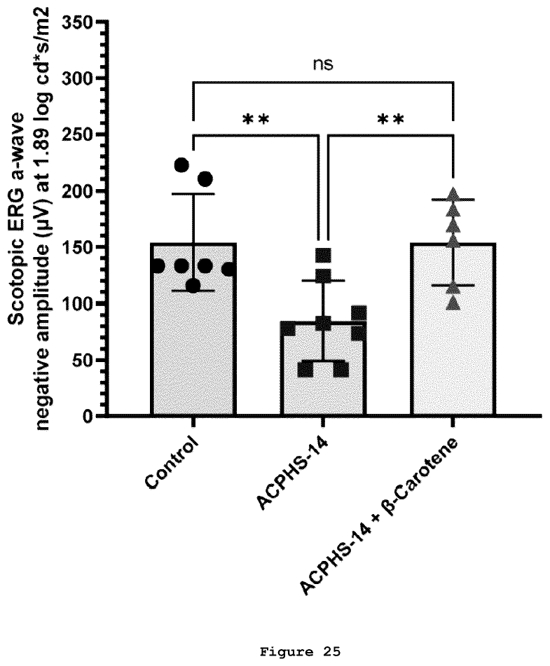

FIG. 25 shows the dynamics of the scotopic ERG a-wave amplitude elicited at the 1.89 log cd*s/m² light intensity in the three groups of mice. As shown in FIG. 17, ACPHS-14 given for 10 days to wild-type mice reduced the a-wave amplitude by 45% in comparison to untreated mice. On the other hand, co-administration of ACPHS-14 with β-carotene induced an increase in a-wave amplitude back to the normal level seen in untreated mice. This data proves that β-carotene can be used as a highly effective “second component” of the present disclosure in implementing the co-drug strategy.

Animal Care and Use Statement: All procedures are in compliance with: the U.S. Department of Agriculture's (USDA) Animal Welfare Act (9 CFR Parts 1, 2, and 3); the Guide for the Care and Use of Laboratory Animals, Institute of Laboratory Animal Resources, National Academy Press, Washington, D.C., 1996; and the National Institutes of Health, Office of Laboratory Animal Welfare. Whenever possible, procedures in this study are designed to avoid or minimize discomfort, distress, and pain to animals.

### Co-Administration of Bispecific RBP4/TTR Ligand ACPHS-52 and C20-D3-Retinyl Acetate Restored the Concentration of Functional Rhodopsin in Mice.

ACPHS-52 was previously identified and described as a compound that exemplifies a novel class of bispecific ligands that act as both RBP4 antagonists and kinetic stabilizers of TTR tetramers (Cioffi, C. L. et al. 2020). Thus, ACPHS-52 was used as a representative “first component” of the present disclosure in order to assess the feasibility of the co-drug strategy. Accordingly, an experiment was conducted to determine whether partial reduction in functional rhodopsin (opsin with conjugated 11-cis-retinal) induced by oral ACPHS-52 in mice can be compensated by co-administration with C20-D3-retinyl acetate. It was previously reported that ACPHS-52 can induce a pronounced reduction in serum RBP4 along with stabilization of TTR tetramers (Cioffi, C. L. et al. 2020).

The experiment was conducted in Balb/c mice. Three groups of mice were used: control untreated mice; mice treated with ACPHS-52; and mice treated with the ACPHS-52 plus C20-D3-retinyl acetate combination. ACPHS-52 was formulated into the chow to ensure a standard daily oral dosing of the compound at a 25 mg/kg dose. C20-D3-retinyl acetate was dissolved in olive oil and was administered through oral gavage daily at a 3.3 mg/kg dose. Dosing duration was 7 days. Following compound dosing, retinal extracts were prepared as previously described (Racz, B. et al. 2018). Rhodopsin was measured spectrophotometrically in retinal extracts (n=8) from dark-adapted treated and control mice using a previously published protocol (Racz, B. et al. 2018).

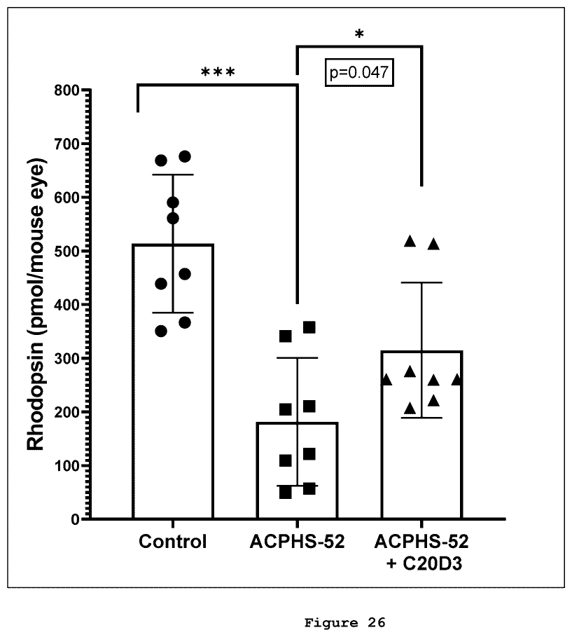

As shown in FIG. 26, ACPHS-52 given for 7 days to wild-type mice induced a 3-fold reduction in rhodopsin in comparison to untreated mice. However, an unpaired t-test on the ungrouped data revealed a statistically significant 1.8-fold increase in rhodopsin concentration in mice co-treated with C20-D3-retinyl acetate and ACPHS-52 in comparison to the ACPHS-52-treated group (P<0.05).

Therefore, co-administration of ACPHS-52 with C20-D3-retinyl acetate restored the concentration of rhodopsin to ~65% of the normal level, proving that deuterated retinoids delivered to the retina by RBP4-independent routes can indeed compensate for the rhodopsin reduction induced by RBP4 lowering.

Partial reduction of rhodopsin associated with RBP4 lowering is at the core of mechanism-based ocular AEs (adverse effects) associated with pharmacological RBP4 reduction, and the experimental results show that co-administration with C20-D3-retinoid can partially restore the level of rhodopsin in ACPHS-52-treated mice.

Animal Care and Use Statement: All procedures are in compliance with: the U.S. Department of Agriculture's (USDA) Animal Welfare Act (9 CFR Parts 1, 2, and 3); the Guide for the Care and Use of Laboratory Animals, Institute of Laboratory Animal Resources, National Academy Press, Washington, D.C., 1996; and the National Institutes of Health, Office of Laboratory Animal Welfare. Whenever possible, procedures in this study are designed to avoid or minimize discomfort, distress, and pain to animals.

### DISCUSSION

The experiments above tested co-administration of β-carotene along with a potent selective TTR ligand, ACPHS-14, that induces a pronounced serum RBP4 reduction in mice. The results show that co-administration of β-carotene can partially restore the concentration of rhodopsin as well as confer functional restoration of the ERG function in the mouse eyes. This proves the feasibility of using β-carotene as the “second component” of the present disclosure in the implementation of the co-drug strategy.

Reduction of rhodopsin levels caused by pharmacological RBP4 reduction is at the core of mechanism-based ocular AEs such as nyctalopia, chromatopsia, and delayed dark adaptation. The present disclosure shows that co-administration of C20-D3-retinyl ester with the compound that induces a pronounced serum RBP4 reduction can partially restore the level of rhodopsin.

The present disclosure constitutes proof of principle and the co-dosing data generated for the TTR ligand and a bispecific compound may be applied to any RBP4-lowering medication, including selective RBPE antagonists.

### REFERENCES

Adams, W. R. et al. Proc. Soc. Exp. Biol. Med. 208, 178-185 (1995).
Aeberli, I. et al. J. Clin. Endocrinol. Metab. 92, 4359-4365 (2007).
Alas, M. et al. J. Med. Chem. 64, 216-232 (2021).
Belite Bio Announces Positive Results from Phase 1 Clinical Trials of LBS-008. 2020; https://www.prnewswire.com/news-releases/belite-bio-announces-positive-results-from-phase-1-clinical-trials-of-lbs-008-301095197.html.
Ben-Shabat, S. et al. J. Biol. Chem. 277, 7183-7190 (2002).
Bergen, H. R. et al. J. Label. Compd. Radiopharm. 25, 11-21 (1988).
Bergmann, M. et al. FASEB J. 18, 562-564 (2004).
Berni, R. & Formelli, F. FEBS Lett. 308, 43-45 (1992).
Birnbach, C. D. et al. Ophthalmology 101, 1211-1219 (1994).
Borhan, B. et al. Chem. Eur. 5, 1172-1175 (1999).
Boyer, N. P. et al. J. Biol. Chem. 287, 22276-22286 (2012).
Brandstetter, C. et al. J. Biol. Chem. 290, 31189-31198 (2015A).
Brandstetter, C. et al. J. Mol. Med. (Berl.) 93, 905-916 (2015B).
Camerini, T. et al. J. Clin. Oncol. 19, 1664-1670 (2001).
Charbel, I. P. et al. Proc. Natl. Acad. Sci. U.S.A. 112, 8415-8420 (2015).
Cioffi, C. L. et al. J. Med. Chem. 57, 7731-7757 (2014).
Cioffi, C. L. et al. J. Med. Chem. 58, 5863-5888 (2015).
Cioffi, C. L. et al. J. Med. Chem. 62, 5470-5500 (2019).
Clagett-Dame, M. & DeLuca, H. F. Annu. Rev. Nutr. 22, 347-381 (2002).
Connelly, S. et al. Current opinion in structural biology 20, 54-62 (2010).
De Laey, J. J. & Verougstraete, C. Retina (Philadelphia, Pa) 15, 399-406 (1995).
De, S. & Sakmar, T. P. J. Gen. Physiol. 120, 147-157 (2002).
Delori, F. C. in G. Coscas FCP, ed. Retinal Pigment Epithelium and Macular Disease (Documenta Ophthalmologica) Vol 62, 37-45 (1995) (Dordrecht, The Netherlands Kluwer Academic Publishers).
Dobri, N. et al. Invest. Ophth. Vis. Sci. 54, 85-95 (2013).
Dontsov, A. E. et al. Dokl. Biochem. Biophys. 425, 98-101 (2009).
Dorey, C. K. et al. Invest. Ophth. Vis. Sci. 30, 1691-1699 (1989).
Eagle, R. C. et al. Ophthalmology 87, 1189-1200 (1980).
Falk, R. H. et al. N. Engl. J. Med. 337, 898-909 (1997).
Finnemann, S. C. et al. Proc. Natl. Acad. Sci. U.S.A. 99, 3842-3847 (2002).
Finniss, M. C. et al. Med. Chem. Commun. 5, 1355-1358 (2014).
Geerlings, M. J. et al. Mol. Immunol. 84, 65-76 (2017).
Gliem, M. et al. JAMA Ophthalmol. 134, 817-824 (2016).
Goswami, B. C. & Barua, A. B. J. Label. Compd. Radiopharm. 46, 843-849 (2003).
Graham, T. E. et al. N. Engl. J. Med. 354, 2552-2563 (2006).
Haskell, M. J. et al. Am. J. Clin. Nutr. 66, 67-74 (1997).
Haskell, M. J. et al. Am. J. Clin. Nutr. 70, 874-880 (1999).
Holz, F. G. et al. Graefes Arch. Clin. Exp. Ophthalmol. 237, 145-152 (1999).
Holz, F. G. et al. Invest. Ophth. Vis. Sci. 42, 1051-1056 (2001).
Holz, F. G. et al. Am. J. Ophthalmol. 143, 463-472 (2007).
Ingelsson, E. et al. Atherosclerosis 206, 239-244 (2009).
Kaufman, Y. et al. J. Biol. Chem. 286, 7958-7965 (2011).
Keller, K. A. et al. Tetrahedron Lett. 46, 1181-1184 (2005).
Kennedy, C. J. et al. Eye (Lond.) 9, 763-771 (1995).
Kiser, P. D. et al. Chem. Rev. 114, 194-232 (2014).
Kowalska, I. et al. J. Clin. Endocrinol. Metab. 93, 2786-2789 (2008).
Lamb, Y. N. & Deeks, E. D. Drugs 79, 863-874 (2019).
Lee, B. P. et al. Macromolecules 39, 1740-1748 (2006).
Lee, S. A. et al. Hepatology 64, 1534-1546 (2016).
Ma, L. et al. J. Biol. Chem. 286, 7966-7974 (2011).
Maeda, A. et al. Mol. Pharmacol. 70, 1220-1229 (2006).
Maeda, A. et al. Nat. Chem. Biol. 8, 170-178 (2012).
Mata, N. L. et al. Retina 33, 498-507 (2013).
Motani, A. et al. J. Biol. Chem. 284, 7673-7680 (2009).
Norseen, J. et al. Mol. Cell. Biol. 32, 2010-2019 (2012).
Nollevaux, M. C. et al. Liver Int. 26, 182-186 (2006).
O'Broin, C. Q. & Guiry, P. J. Org. Lett. 22, 879-883 (2020).
Palczewski, K. Trends Pharmacol. Sci. 31, 284-295 (2010).
Park, J. et al. Ann. Pharmacother. 54, 470-477 (2020).
Petrukhin, K. Expert Opin. Ther. Tar. 11, 625-639 (2007).
Petrukhin, K. Drug Discov. Today Ther. Strateg. 10, e11-e20 (2013).
Petrukhin, K. Recent Developments in Agents for the Treatment of Age-Related Macular Degeneration and Stargardt Disease. In: Cioffi, C. L. ed. Drug Delivery Challenges and Novel Therapeutic Approaches for Retinal Diseases. Cham: Springer International Publishing, 125-160 (2020).
Qi, Q. et al. J. Clin. Endocrinol. Metab. 92, 4827-4834 (2007).
Racz, B. et al. J. Biol. Chem. 293, 11574-11588 (2018).
Racz, B. et al. PLOS One 15, e0228291 (2020).
Radu, R. A. et al. Proc. Natl. Acad. Sci. U.S.A. 100, 4742-4747 (2003).
Radu, R. A. et al. Invest. Ophth. Vis. Sci. 46, 4393-4401 (2005).
Radu, R. A. et al. J. Biol. Chem. 286, 18593-18601 (2011).
Radu, R. A. et al. J. Biol. Chem. 289, 9113-9120 (2014).
Rozanowska, M. et al. J. Biol. Chem. 270, 18825-18830 (1995).
Schmitz-Valckenberg, S. et al. Surv. Ophthalmol. 54, 96-117 (2009).
Smith, R. T. et al. Invest. Ophthalmol. Vis. Sci. 54, 5543 (2013).
Sparrow, J. R. et al. Invest. Ophth. Vis. Sci. 40, 2988-2995 (1999).
Sparrow, J. R. & Cai, B. Invest. Ophth. Vis. Sci. 42, 1356-1362 (2001).
Sparrow, J. R. et al. Invest. Ophth. Vis. Sci. 43, 1222-1227 (2002).
Sparrow, J. R. et al. Vis. Res. 43, 2983-2990 (2003).
Sparrow, J. R. & Boulton, M. Exp. Eye Res. 80, 595-606 (2005).
Sparrow, J. R. et al. Prog. Retin. Eye Res. 31, 121-135 (2012).
Sparrow, J. R. et al. Invest. Ophthalmol. Vis. Sci. 54, 8325-8326 (2013).
Steinmetz, A. C. et al. Annu. Rev. Biophys. Biomol. Struct. 30, 329-359 (2001).
Su, Z. et al. Acta Pharm. Sin. B. 11, 3889-3907 (2021).
Suter, M. et al. J. Biol. Chem. 275, 39625-39630 (2000).
Tang, P. H. et al. Prog. Retin. Eye Res. (2012).
Tsin, A. et al. J. Biol. Chem. 293, 13016-13021 (2018).
Tsuchikama, K. & An, Z. Protein Cell 9, 33-46 (2018).
Vives-Bauza, C. et al. J. Biol. Chem. 283, 24770-24780 (2008).
Vogel, S. et al. Biochemistry 41, 15360-15368 (2002).
Washington, I. et al. Adv. Exp. Med. Biol. 854, 347-353 (2016).
Weng, J. et al. Cell 98, 13-23 (1999).
Wolf, G. Physiological Rev. 64, 873-937 (1984).
Yang, Q. et al. Nature 436, 356-362 (2005).
Young, R. W. Surv. Ophthalmol. 31, 291-306 (1987).
Zhou, J. et al. Proc. Natl. Acad. Sci. U.S.A. 103, 16182-16187 (2006).

1. A compound having the general structure (P-)ₐM(-Q)_{b} or P(-Q)_{b}, wherein:
P represents a single-valence group formed by eliminating a hydrogen atom bonded to a heteroatom or by eliminating a hydroxyl group from a selective RBP4 antagonist;
a is 1, 2, 3, 4, or 5;
Q represents a single-valence group formed by eliminating a hydrogen atom bonded to a heteroatom or by eliminating a hydroxyl group from a C20-D3-visual-chromophore-producing compound;
b is 1, 2, 3, 4, or 5;
M represents an (a+b)-valence group comprising carbon, hydrogen, nitrogen, and oxygen atoms;
each P in (P-)ₐM(-Q)_{b} is independently bonded to M via an ester, carbonyl, peptide, carbamate, or hemiaminal linkage that is cleavable at an acidic pH or is enzymatically cleavable;
each Q in (P-)ₐM(-Q)_{b} is independently bonded to M via a carbonate, carbonyl, peptide, silyl ether, or ester linkage that is cleavable at an acidic pH or is enzymatically cleavable;
each Q in P(-Q)_{b} is independently bonded to P via an ester, carbonyl, peptide, carbamate, or hemiaminal linkage that is cleavable at an acidic pH or is enzymatically cleavable;
the selective RBP4 antagonist is a compound having the structure: 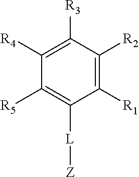
wherein L is a linking group having the structure 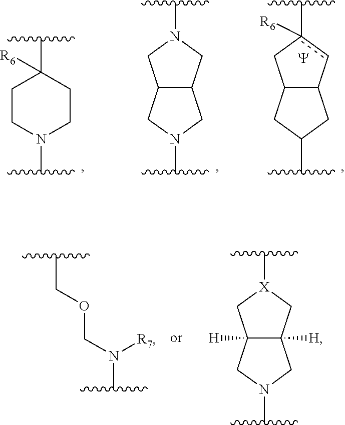
and
Z is a group having the structure 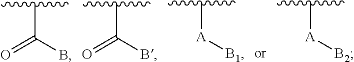
wherein
R₁, R₂, R₃, R₄, and R₅ are each independently H, halogen, CF₃, C₁-C₄ alkyl, aryl, or heteroaryl;
R₆ is H, OH, or halogen, or is absent;
ψ is absent or present, and when present, is a bond;
B is a substituted or unsubstituted heterobicycle, pyridazine, pyrazole, pyrazine, thiadiazole, or triazole, wherein
the heterobicycle is other than chloro-substituted indole; and
the pyrazole, when substituted, is substituted with other than trifluoromethyl;
B′ is a substituted or unsubstituted phenyl, pyridine, pyrimidine, benzyl, pyrrolidine, sulfolane, oxetane, CO₂H, or (C₁-C₄ alkylenyl)-CO₂H, wherein
the substituted phenyl is substituted with other than trifluoromethyl or 3-(methyl carboxylate),
the substituted pyridine is substituted with other than trifluoromethyl,
the substituted pyrrolidine is substituted with other than hydroxamic acid, and
the substituted or unsubstituted pyrrolidine is bound to the carbonyl through a carbon-carbon bond;
A is absent or present, and when present, is 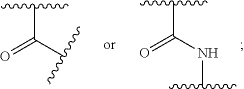
B₁ is a substituted or unsubstituted monocycle, bicycle, heteromonocycle, heterobicycle, benzyl, CO₂H, or (C₁-C₄ alkylenyl)-CO₂H,
wherein when B₁ is CO₂H, then A is present and is 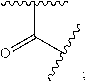 [SMILES: *C(*)=O]
R₇ is alkyl;
X is N or CR₈,
wherein R₈ is H, OH, or halogen; and
B₂ has the structure: 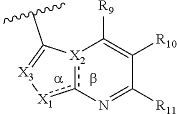
wherein
α and β are each a bond that is present or absent;
X₁ is N, NH, or NR₉₉,
wherein R₉₉ is alkyl, alkenyl, or alkynyl;
X₂ is C or N;
X₃ is CH or N;
R₉, R₁₀, and R₁₁ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃, wherein
X₁, X₂, and X₃ are each N, α is present, and β is absent; or
X₁ is NH, X₂ is C, X₃ is CH, α is absent, and β is present; or
X₁ is N, X₂ is N, X₃ is CH, α is present, and β is absent; or
X₁ is NH or NR₉₉, X₂ is C, X₃ is N, α is absent, and β is present, wherein when X₁ is NH, X₂ is C, X₃ is N, α is absent, and β is present, then one of R₉, R₁₀, and R₁₁ is other than H;
or
B₂ has the structure: 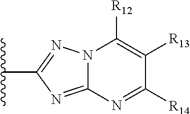
wherein
R₁₂, R₁₃, and R₁₄ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
or a pharmaceutically acceptable salt thereof; and
the C20-D3-visual-chromophore-producing compound is selected from the group consisting of C20-D3-retinol, C20-D3-9-cis-retinol, C20-D3-11-cis-retinol, and C20-D3-C20′-D3-β-carotene.

2. The compound of claim 1 having the general structure (P-)ₐM(-Q)_{b} or P(-Q)_{b}, wherein:
P represents a single-valence group formed by eliminating a hydrogen atom bonded to a heteroatom or by eliminating a hydroxyl group from a selective RBP4 antagonist;
a is 1, 2, 3, 4, or 5;
Q represents a single-valence group formed by eliminating a hydrogen atom bonded to a heteroatom or by eliminating a hydroxyl group from a C20-D3-visual-chromophore-producing compound;
b is 1, 2, 3, 4, or 5;
M represents an (a+b)-valence group comprising carbon, hydrogen, and oxygen atoms;
each P in (P-)ₐM(-Q)_{b} is independently bonded to M via an ester, carbamate, or hemiaminal linkage that is cleavable at an acidic pH or is enzymatically cleavable;
each Q in (P-)ₐM(-Q)_{b} is independently bonded to M via a carbonate, silyl ether, or ester linkage that is cleavable at an acidic pH or is enzymatically cleavable;
each Q in P(-Q)_{b} is independently bonded to P via an ester, carbamate, or hemiaminal linkage that is cleavable at an acidic pH or is enzymatically cleavable;
the selective RBP4 antagonist is a compound having the structure: 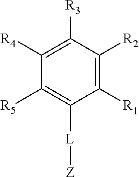
wherein L is a linking group having the structure 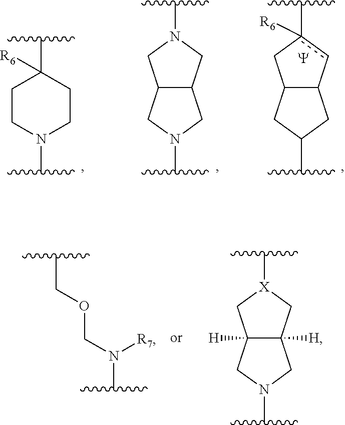
and
Z is a group having the structure 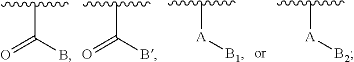
wherein
R₁, R₂, R₃, R₄, and R₅ are each independently H, halogen, CF₃, C₁-C₄ alkyl, aryl, or heteroaryl;
R₆ is H, OH, or halogen, or is absent;
ψ is absent or present, and when present, is a bond;
B is a substituted or unsubstituted heterobicycle, pyridazine, pyrazole, pyrazine, thiadiazole, or triazole, wherein
the heterobicycle is other than chloro-substituted indole; and
the pyrazole, when substituted, is substituted with other than trifluoromethyl;
B′ is a substituted or unsubstituted phenyl, pyridine, pyrimidine, benzyl, pyrrolidine, sulfolane, oxetane, CO₂H, or (C₁-C₄ alkylenyl)-CO₂H, wherein
the substituted phenyl is substituted with other than trifluoromethyl or 3-(methyl carboxylate),
the substituted pyridine is substituted with other than trifluoromethyl,
the substituted pyrrolidine is substituted with other than hydroxamic acid, and
the substituted or unsubstituted pyrrolidine is bound to the carbonyl through a carbon-carbon bond;
A is absent or present, and when present, is 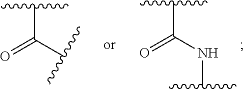
B₁ is a substituted or unsubstituted monocycle, bicycle, heteromonocycle, heterobicycle, benzyl, CO₂H, or (C₁-C₄ alkylenyl)-CO₂H,
wherein when B₁ is CO₂H, then A is present and is 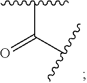 [SMILES: *C(*)=O]
R₇ is alkyl;
X is N or CR₈,
wherein R₈ is H, OH, or halogen; and
B₂ has the structure: 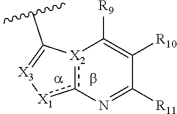
wherein
α and β are each a bond that is present or absent;
X₁ is N, NH, or NR₉₉,
wherein R₉₉ is alkyl, alkenyl, or alkynyl;
X₂ is C or N;
X₃ is CH or N;
R₉, R₁₀, and R₁₁ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃, wherein
X₁, X₂, and X₃ are each N, α is present, and β is absent; or
X₁ is NH, X₂ is C, X₃ is CH, α is absent, and β is present; or
X₁ is N, X₂ is N, X₃ is CH, α is present, and β is absent; or
X₁ is NH or NR₉₉, X₂ is C, X₃ is N, α is absent, and β is present, wherein when X₁ is NH, X₂ is C, X₃ is N, α is absent, and β is present, then one of R₉, R₁₀, and R₁₁ is other than H;
or
B₂ has the structure: 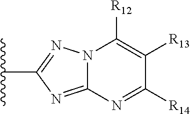
wherein
R₁₂, R₁₃, and R₁₄ are each independently H, halogen, alkyl, alkenyl, alkynyl, alkylenyl-OH, alkylenyl-NH₂, alkylenyl-OAc, alkylenyl-O(CO)-alkyl, alkylenyl-O-alkyl, haloalkyl, cycloalkyl, O-alkyl, NH-alkyl, C(O)OH, C(O)—NH₂, C(O)—N(CH₃)₂, C(O)—NHCH₃, NHC(O)—N(CH₃)₂, CN, or CF₃;
or a pharmaceutically acceptable salt thereof; and
the C20-D3-visual-chromophore-producing compound is selected from the group consisting of C20-D3-retinol, C20-D3-9-cis-retinol, C20-D3-11-cis-retinol, and C20-D3-C20′-D3-β-carotene.

3. The compound of claim 1, wherein the selective RBP4 antagonist is the compound wherein R₁, R₂, R₃, R₄, and R₅ are each independently H, halogen, CF₃, or C₁-C₄ alkyl.

4. The compound of claim 1, wherein
(a) the selective RPB4 antagonist is the compound wherein L is 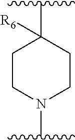
and Z is 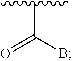
(b) the selective RBP4 antagonist is the compound wherein L is 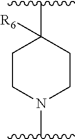
and Z is 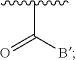
(c) the selective RBP4 antagonist is the compound wherein
L is 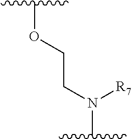
and Z is 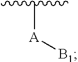
(d) the selective RBP4 antagonist is the compound wherein
L is 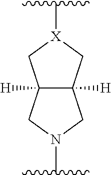
and Z is 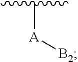
(e) the selective RBP4 antagonist is the compound wherein L is 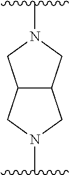 [SMILES: *N1CC2CN(*)CC2C1]
and Z is 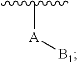
or
(f) the selective RBP4 antagonist is the compound wherein
L is 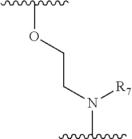
and Z is

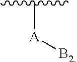

5. The compound of claim 1, wherein the selective RBP4 antagonist is the compound wherein
L is 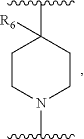
Z is 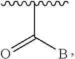
two or more of R₁, R₂, R₃, R₄, and R₅ are other than H, and when
R₁ is CF₃, R₂ is H, R₃ is F, R₄ is H, and R₅ is H, or
R₁ is H, R₂ is CF₃, R₃ is H, R₄ is CF₃, and R₅ is H, or
R₁ is Cl, R₂ is H, R₃ is H, R₄ is F, and R₅ is H, or
R₁ is CF₃, R₂ is H, R₃ is F, R₄ is H, and R₅ is H, or
R₁ is CF₃, R₂ is F, R₃ is H, R₄ is H, and R₅ is H, or
R₁ is Cl, R₂ is F, R₃ is H, R₄ is H, and R₅ is H,
then B is other than

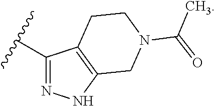 [SMILES: *c1n[nH]c2c1CCN(C(C)=O)C2]

6. The compound of claim 1, wherein the selective RBP4 antagonist is the compound wherein
L is 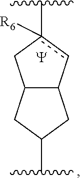
Z is 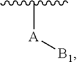
R₆ is absent or present, and when present, is H, OH, or halogen, and
when ψ is present, then R₆ is absent, and when y is absent, then R₆ is present.

7. The compound of claim 1, wherein the selective RBP4 antagonist is the compound wherein
L is 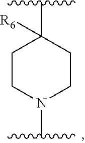
Z is 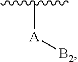
R₆ is H, and
A is

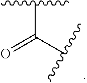 [SMILES: *C(*)=O]

8. The compound of claim 1, wherein the selective RBP4 antagonist is 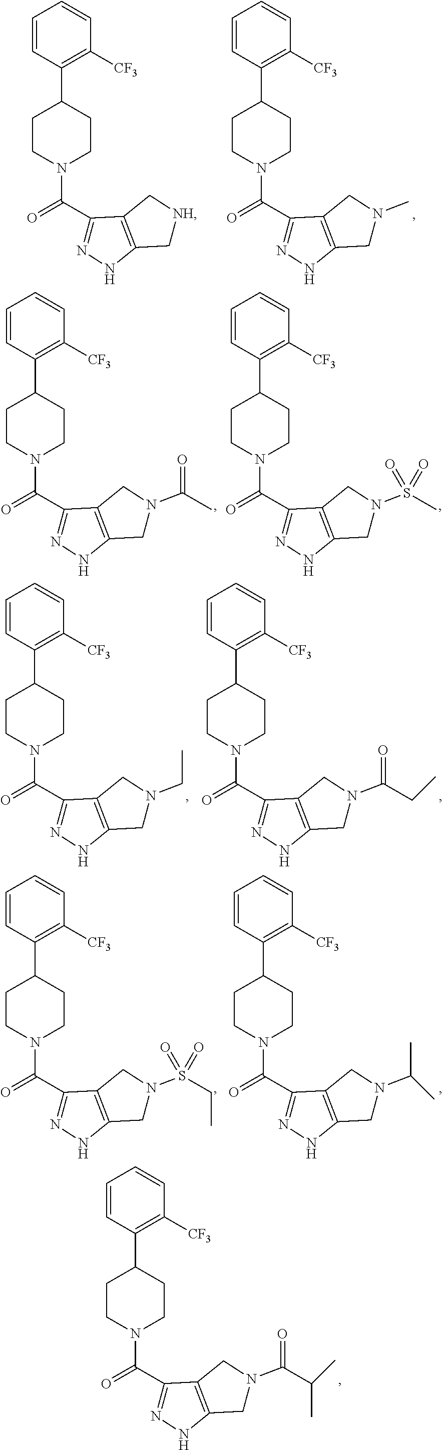 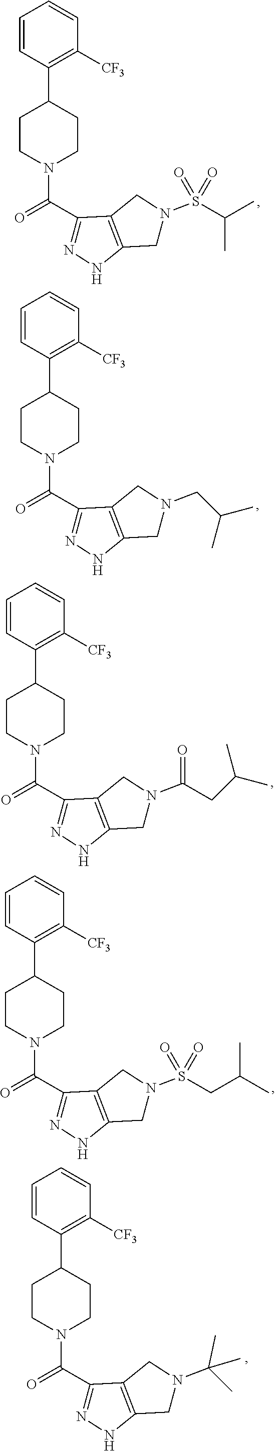 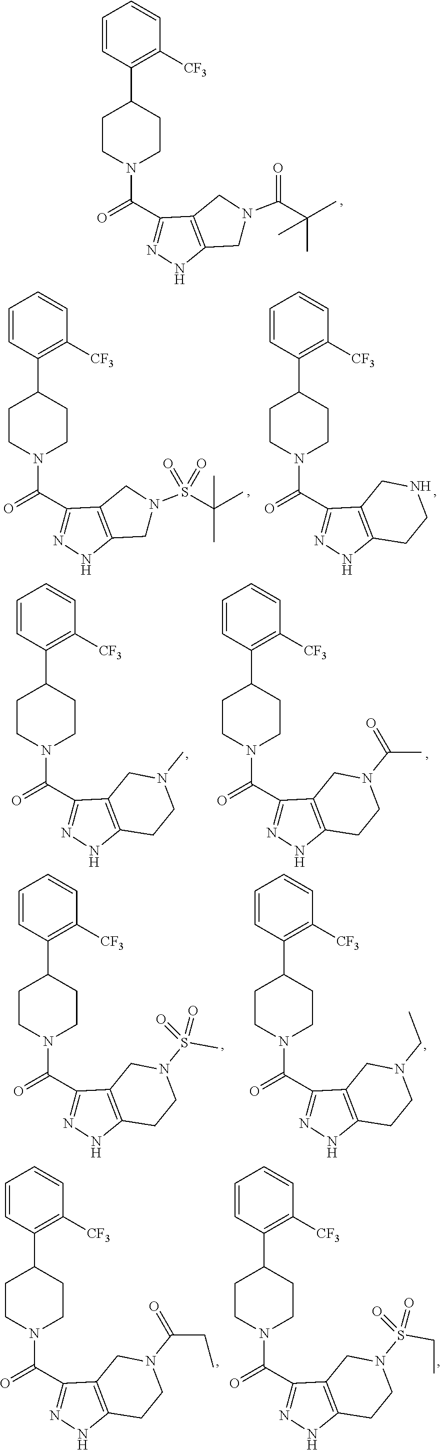 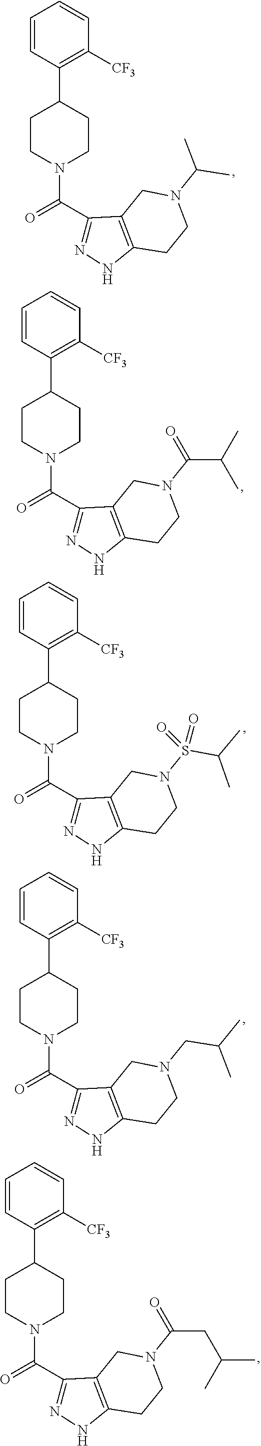 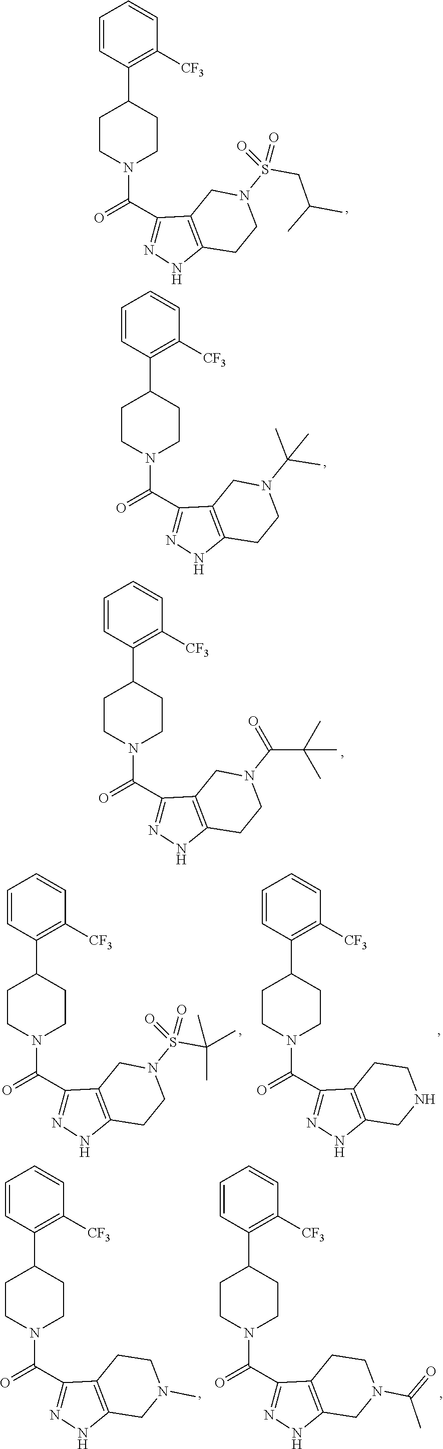 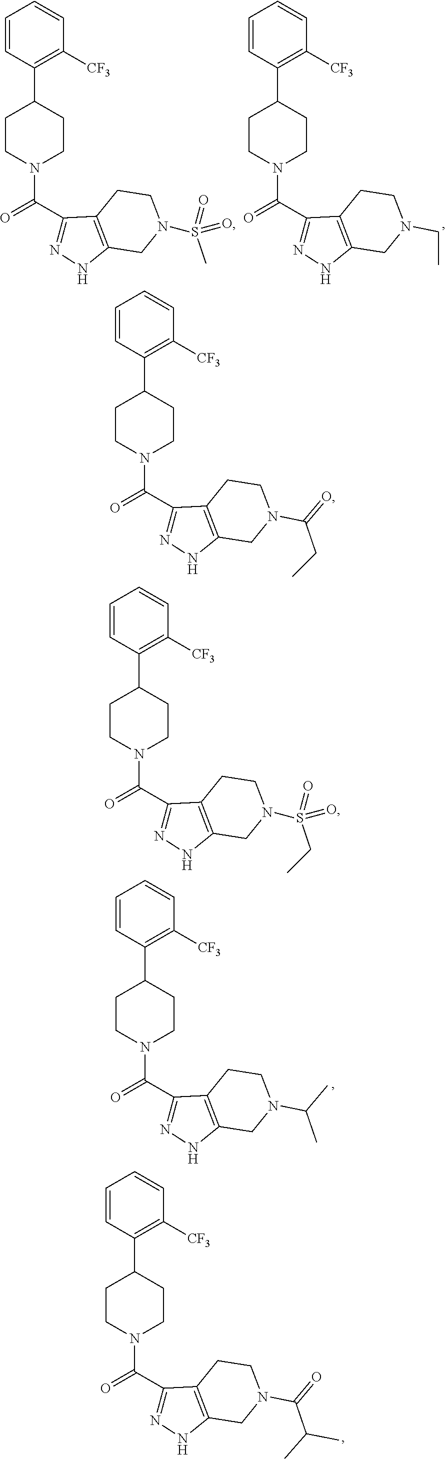 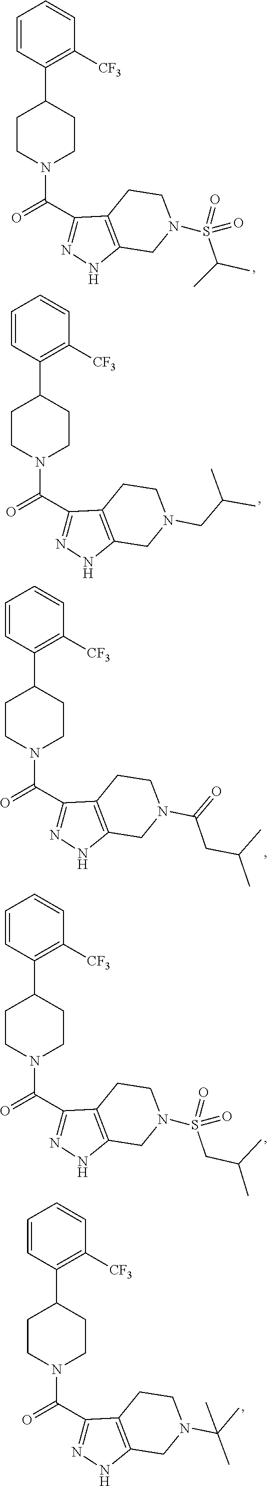 [SMILES: CC(C)S(=O)(=O)N1CCc2c(C(=O)N3CCC(c4ccccc4C(F)(F)F)CC3)n[nH]c2C1 ; CC(C)CN1CCc2c(C(=O)N3CCC(c4ccccc4C(F)(F)F)CC3)n[nH]c2C1 ; CC(C)CC(=O)N1CCc2c(C(=O)N3CCC(c4ccccc4C(F)(F)F)CC3)n[nH]c2C1 ; CC(C)CS(=O)(=O)N1CCc2c(C(=O)N3CCC(c4ccccc4C(F)(F)F)CC3)n[nH]c2C1 ; CC(C)(C)N1CCc2c(C(=O)N3CCC(c4ccccc4C(F)(F)F)CC3)n[nH]c2C1] 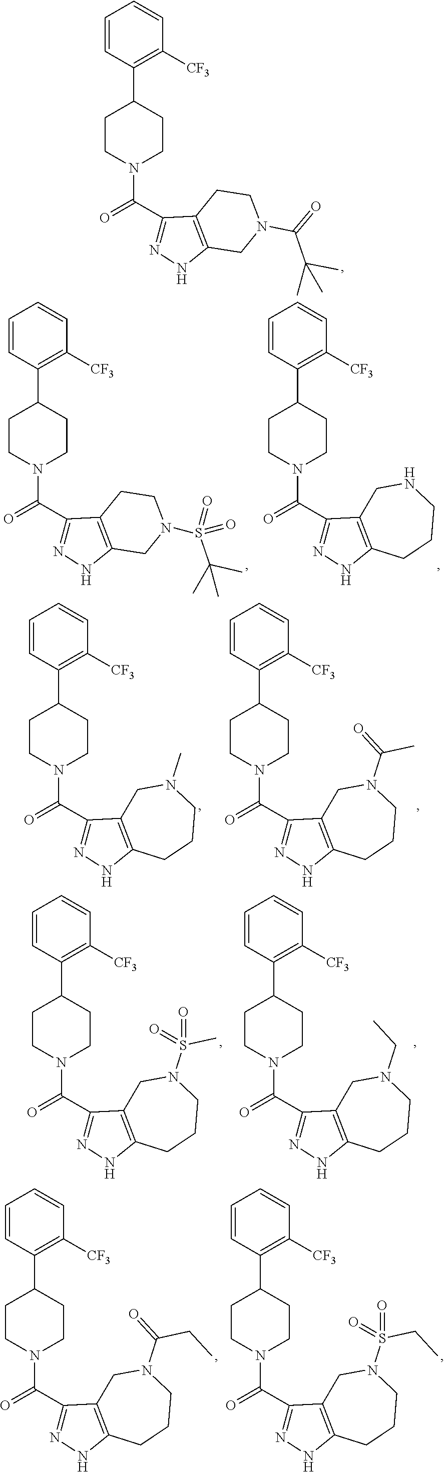 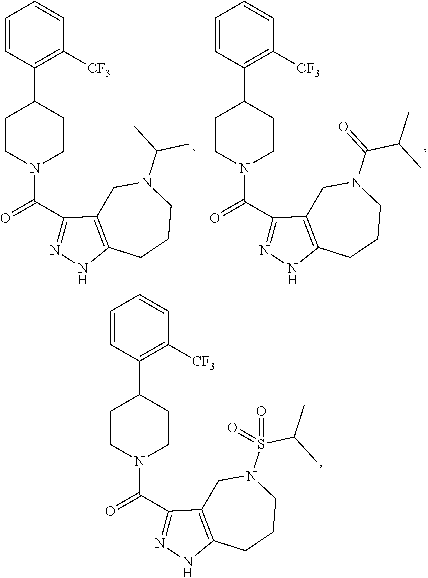 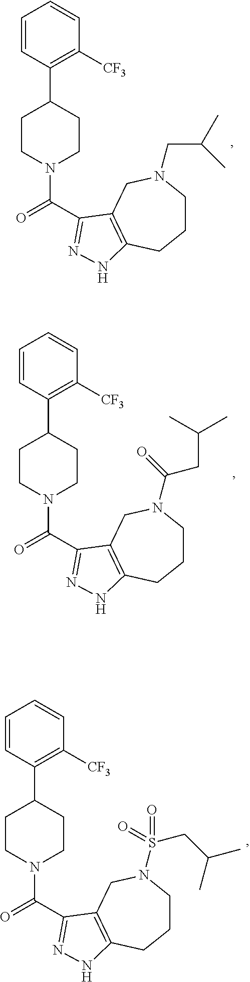 [SMILES: CC(C)CN1CCCc2[nH]nc(C(=O)N3CCC(c4ccccc4C(F)(F)F)CC3)c2C1 ; CC(C)CC(=O)N1CCCc2[nH]nc(C(=O)N3CCC(c4ccccc4C(F)(F)F)CC3)c2C1 ; CC(C)CS(=O)(=O)N1CCCc2[nH]nc(C(=O)N3CCC(c4ccccc4C(F)(F)F)CC3)c2C1] 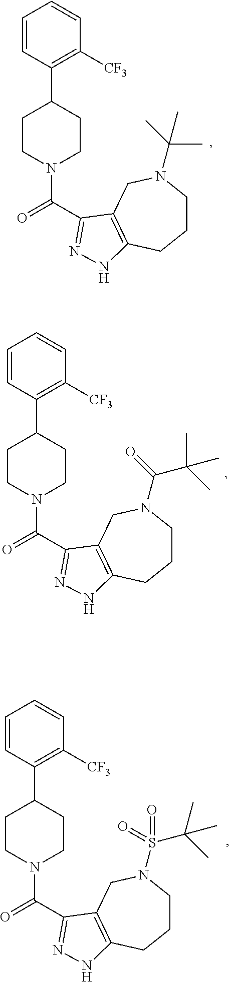 [SMILES: CC(C)(C)N1CCCc2[nH]nc(C(=O)N3CCC(c4ccccc4C(F)(F)F)CC3)c2C1 ; CC(C)(C)C(=O)N1CCCc2[nH]nc(C(=O)N3CCC(c4ccccc4C(F)(F)F)CC3)c2C1 ; CC(C)(C)S(=O)(=O)N1CCCc2[nH]nc(C(=O)N3CCC(c4ccccc4C(F)(F)F)CC3)c2C1] 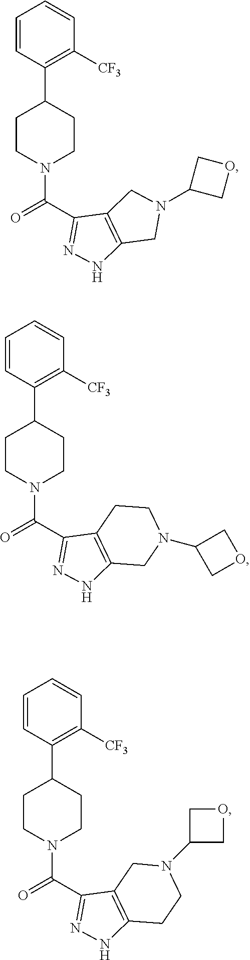 [SMILES: O=C(c1n[nH]c2c1CN(C1COC1)C2)N1CCC(c2ccccc2C(F)(F)F)CC1 ; O=C(c1n[nH]c2c1CCN(C1COC1)C2)N1CCC(c2ccccc2C(F)(F)F)CC1 ; O=C(c1n[nH]c2c1CN(C1COC1)CC2)N1CCC(c2ccccc2C(F)(F)F)CC1] 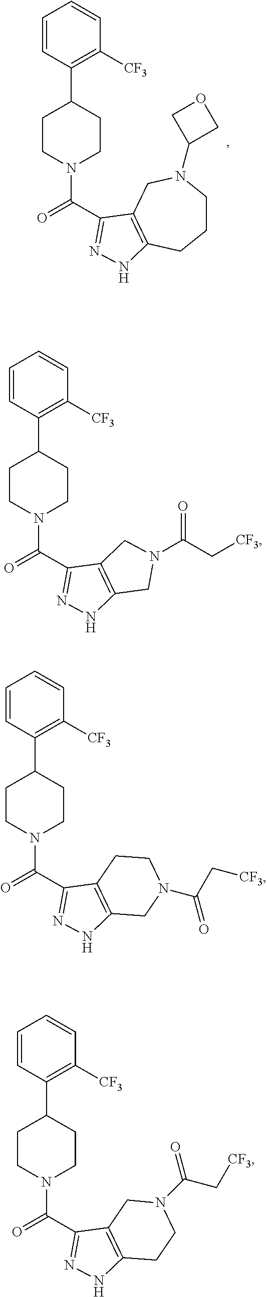 [SMILES: O=C(c1n[nH]c2c1CN(C1COC1)CCC2)N1CCC(c2ccccc2C(F)(F)F)CC1 ; O=C(CC(F)(F)F)N1Cc2[nH]nc(C(=O)N3CCC(c4ccccc4C(F)(F)F)CC3)c2C1 ; O=C(CC(F)(F)F)N1CCc2c(C(=O)N3CCC(c4ccccc4C(F)(F)F)CC3)n[nH]c2C1 ; O=C(CC(F)(F)F)N1CCc2[nH]nc(C(=O)N3CCC(c4ccccc4C(F)(F)F)CC3)c2C1] 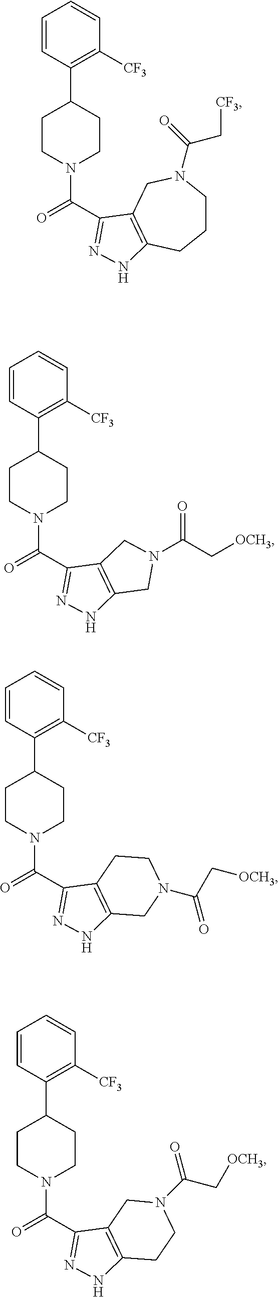 [SMILES: O=C(CC(F)(F)F)N1CCCc2[nH]nc(C(=O)N3CCC(c4ccccc4C(F)(F)F)CC3)c2C1 ; COCC(=O)N1Cc2[nH]nc(C(=O)N3CCC(c4ccccc4C(F)(F)F)CC3)c2C1 ; COCC(=O)N1CCc2c(C(=O)N3CCC(c4ccccc4C(F)(F)F)CC3)n[nH]c2C1 ; COCC(=O)N1CCc2[nH]nc(C(=O)N3CCC(c4ccccc4C(F)(F)F)CC3)c2C1] 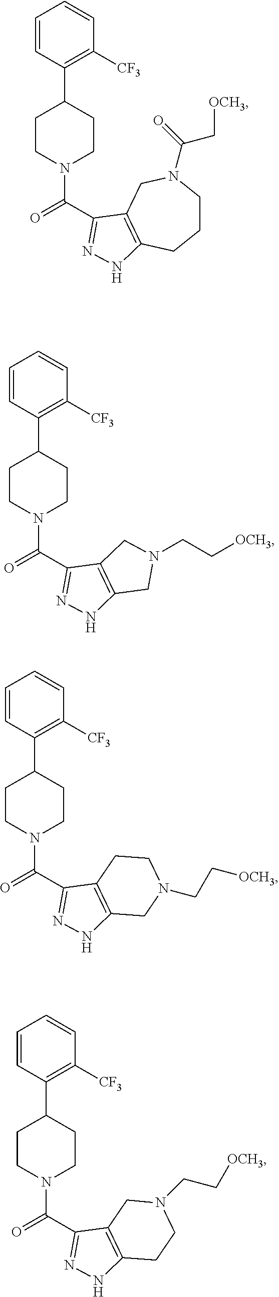 [SMILES: COCC(=O)N1CCCc2[nH]nc(C(=O)N3CCC(c4ccccc4C(F)(F)F)CC3)c2C1 ; COCCN1Cc2[nH]nc(C(=O)N3CCC(c4ccccc4C(F)(F)F)CC3)c2C1 ; COCCN1CCc2c(C(=O)N3CCC(c4ccccc4C(F)(F)F)CC3)n[nH]c2C1 ; COCCN1CCc2[nH]nc(C(=O)N3CCC(c4ccccc4C(F)(F)F)CC3)c2C1] 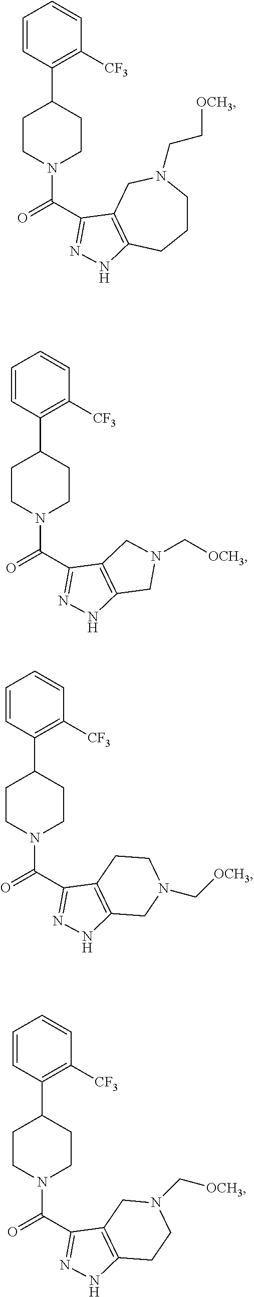 [SMILES: COCCN1CCCc2[nH]nc(C(=O)N3CCC(c4ccccc4C(F)(F)F)CC3)c2C1 ; COCN1Cc2[nH]nc(C(=O)N3CCC(c4ccccc4C(F)(F)F)CC3)c2C1 ; COCN1CCc2c(C(=O)N3CCC(c4ccccc4C(F)(F)F)CC3)n[nH]c2C1 ; COCN1CCc2[nH]nc(C(=O)N3CCC(c4ccccc4C(F)(F)F)CC3)c2C1] 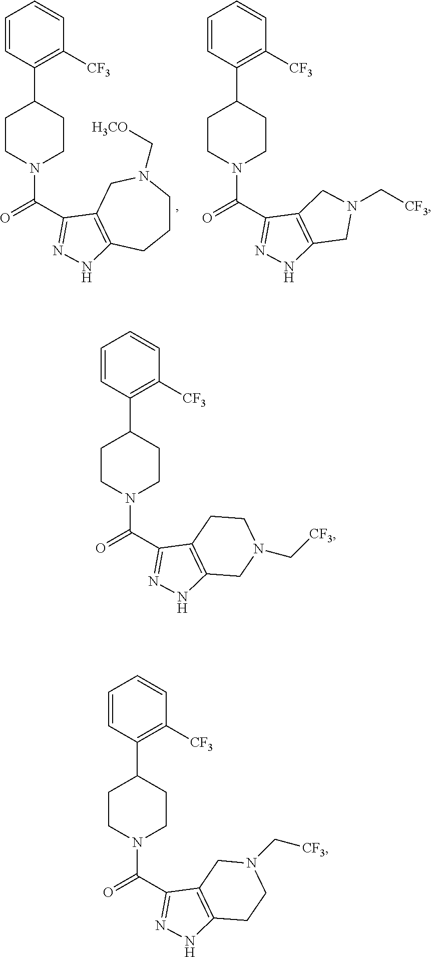 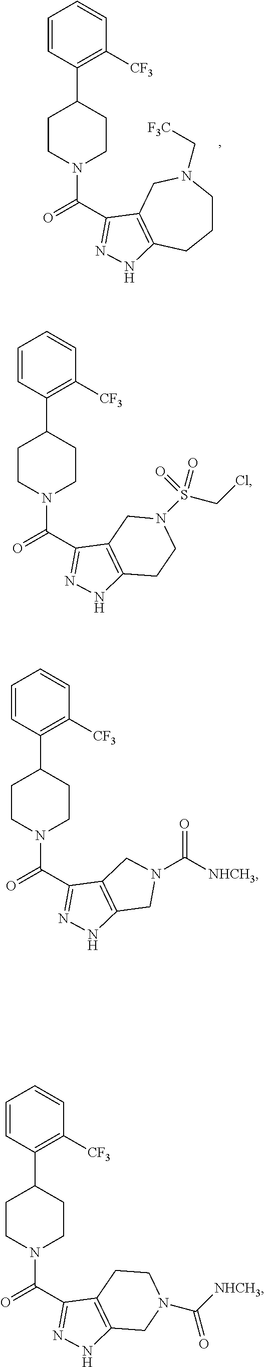 [SMILES: O=C(c1n[nH]c2c1CN(CC(F)(F)F)CCC2)N1CCC(c2ccccc2C(F)(F)F)CC1 ; O=C(c1n[nH]c2c1CN(S(=O)(=O)CCl)CC2)N1CCC(c2ccccc2C(F)(F)F)CC1 ; CNC(=O)N1Cc2[nH]nc(C(=O)N3CCC(c4ccccc4C(F)(F)F)CC3)c2C1 ; CNC(=O)N1CCc2c(C(=O)N3CCC(c4ccccc4C(F)(F)F)CC3)n[nH]c2C1] 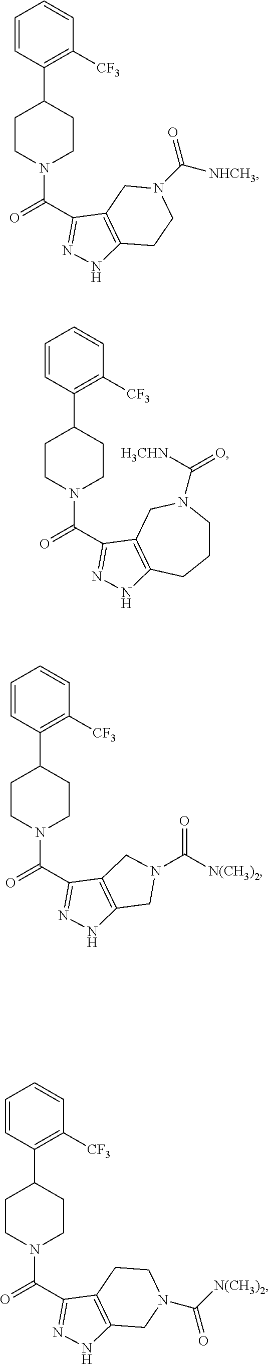 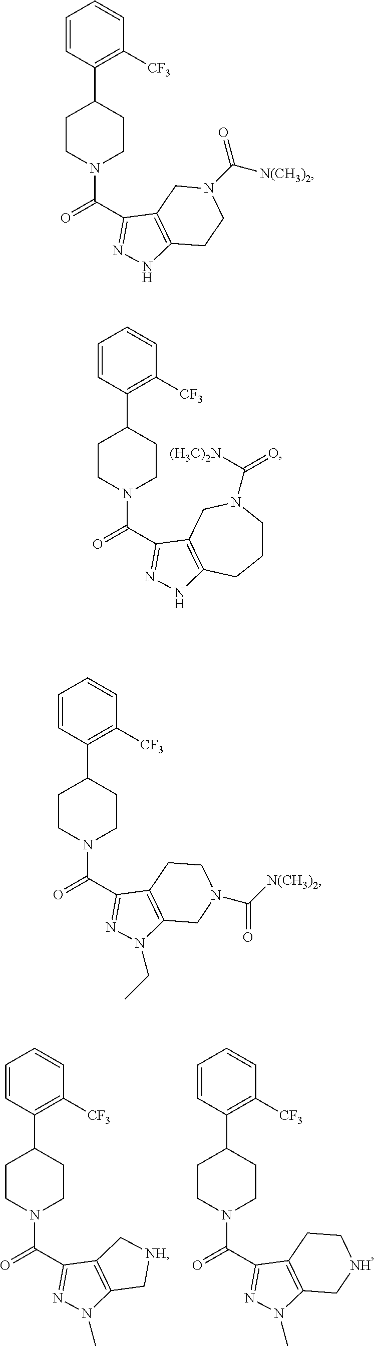 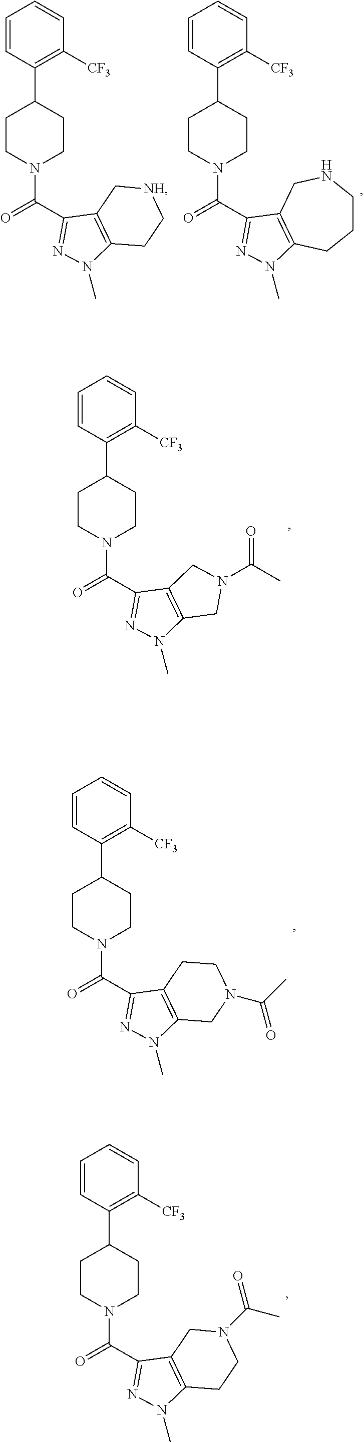 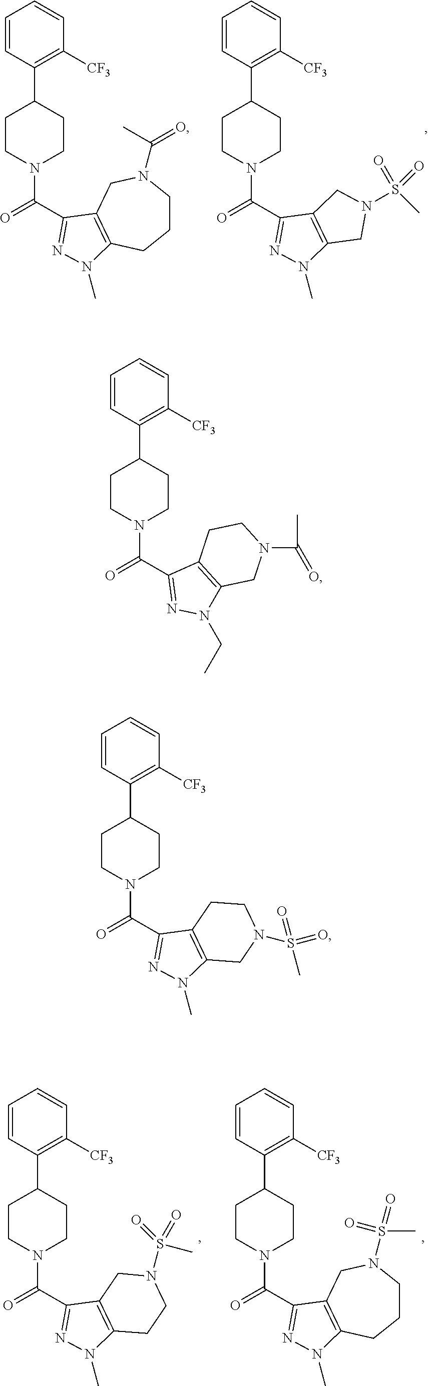 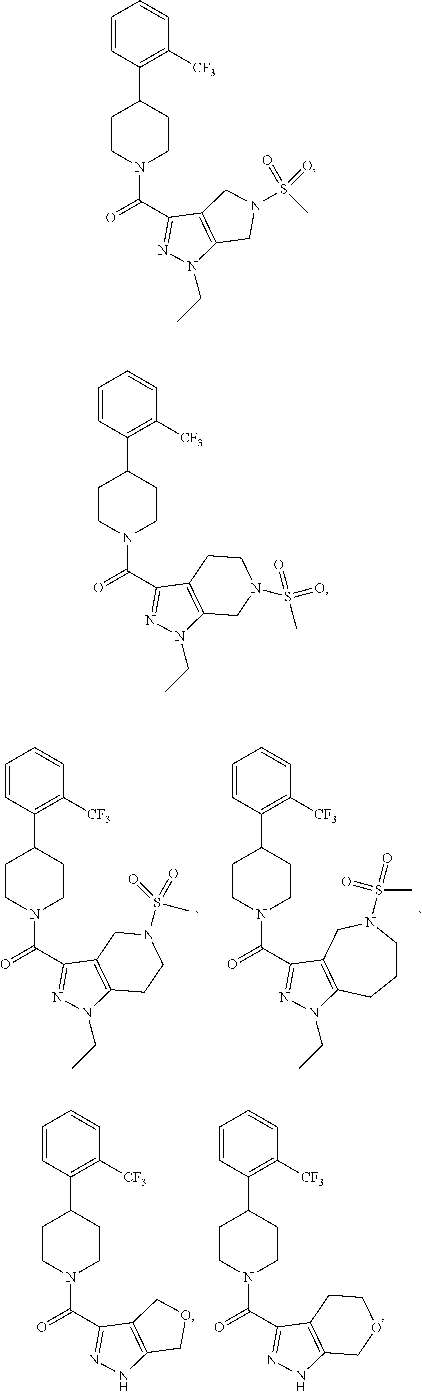 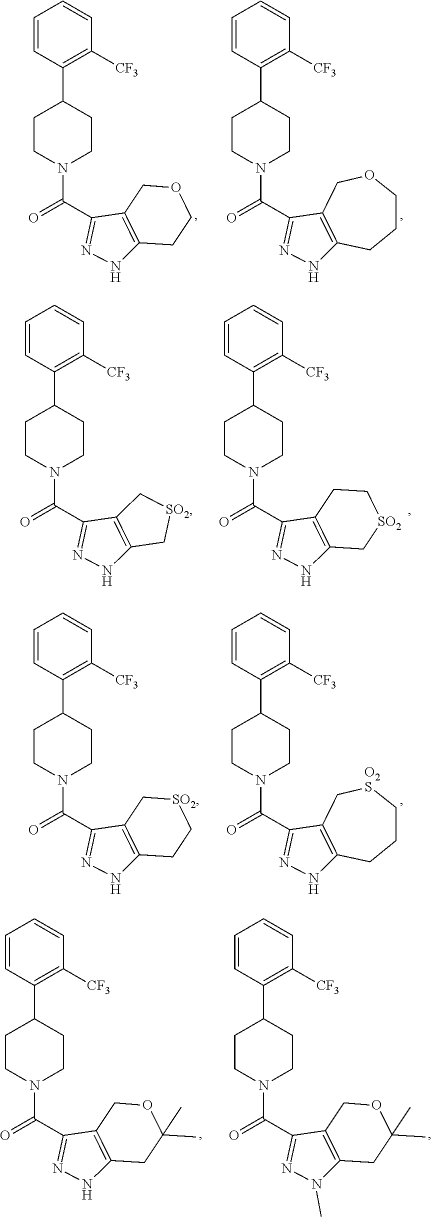 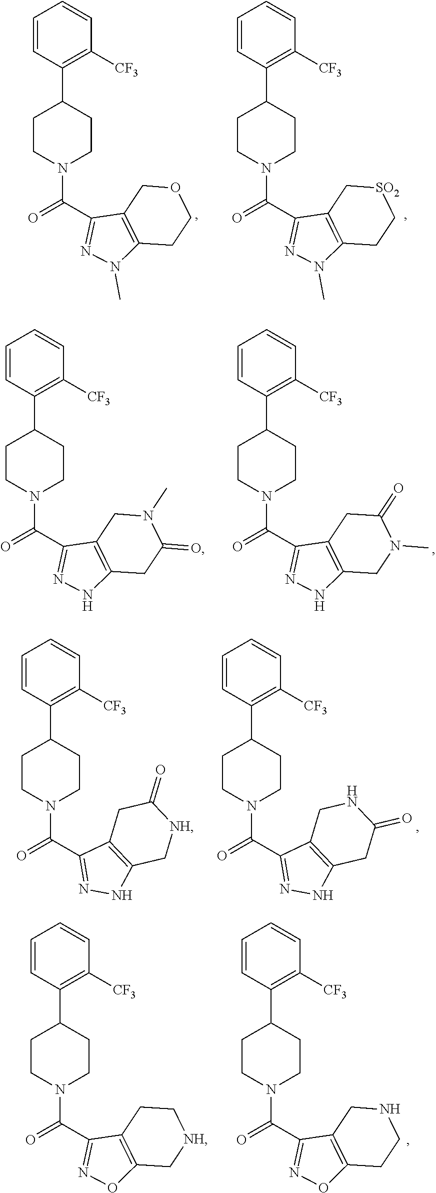 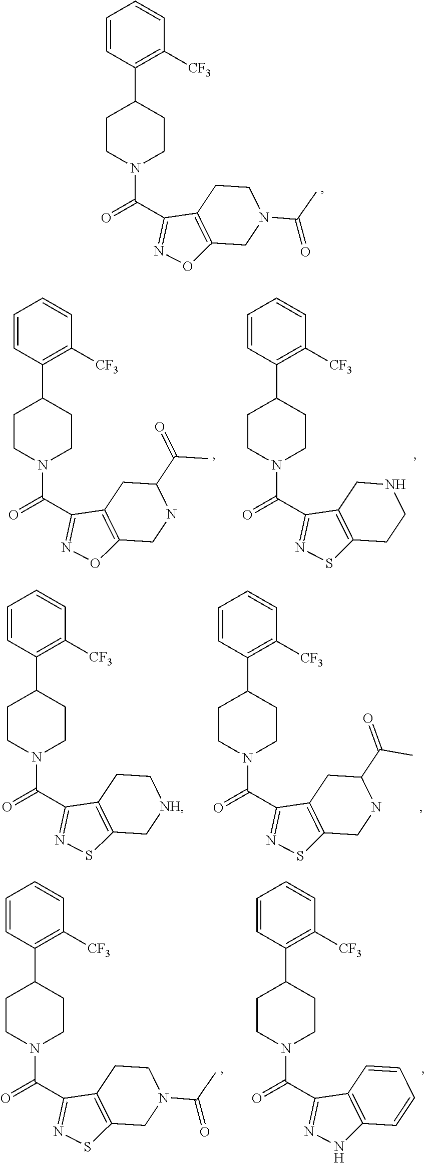 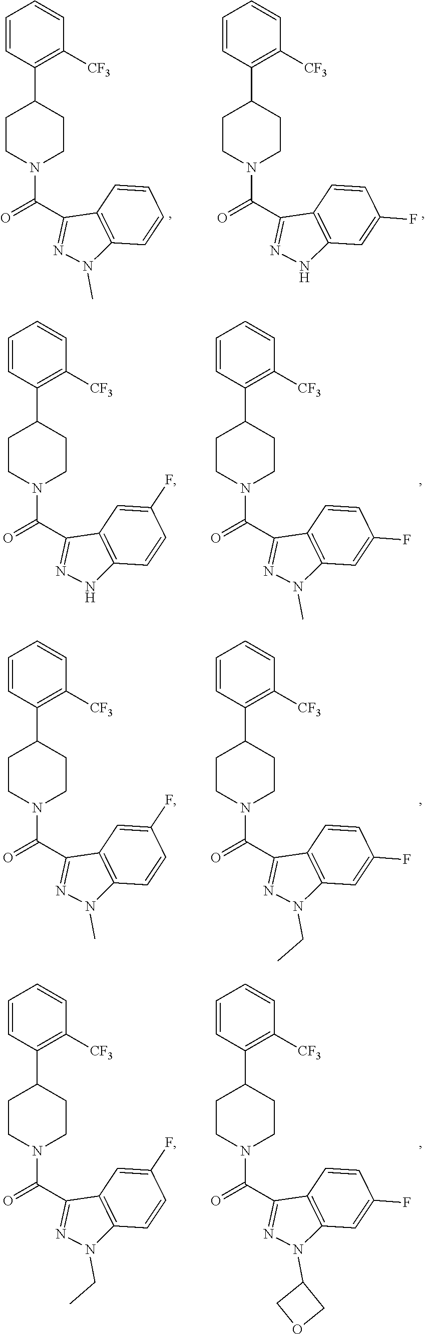 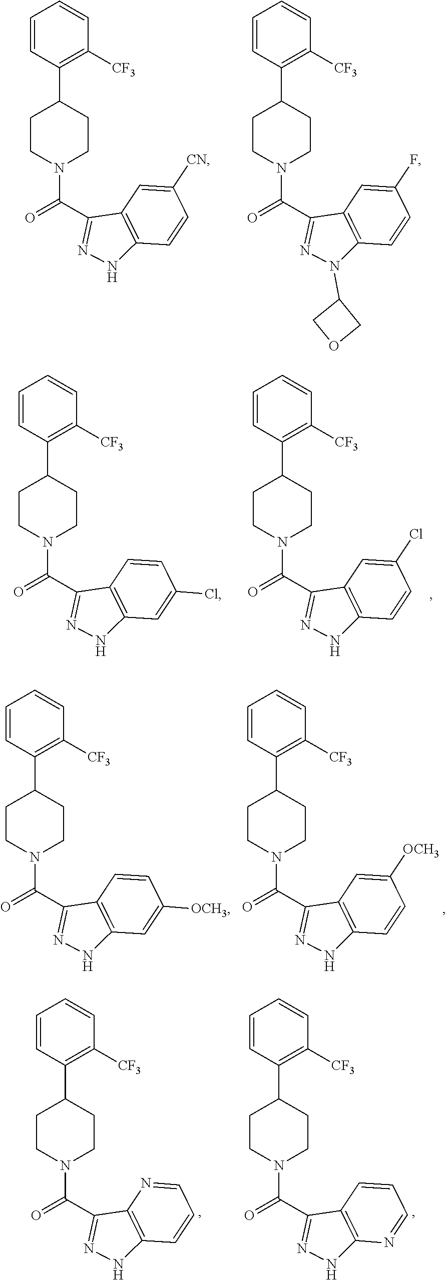 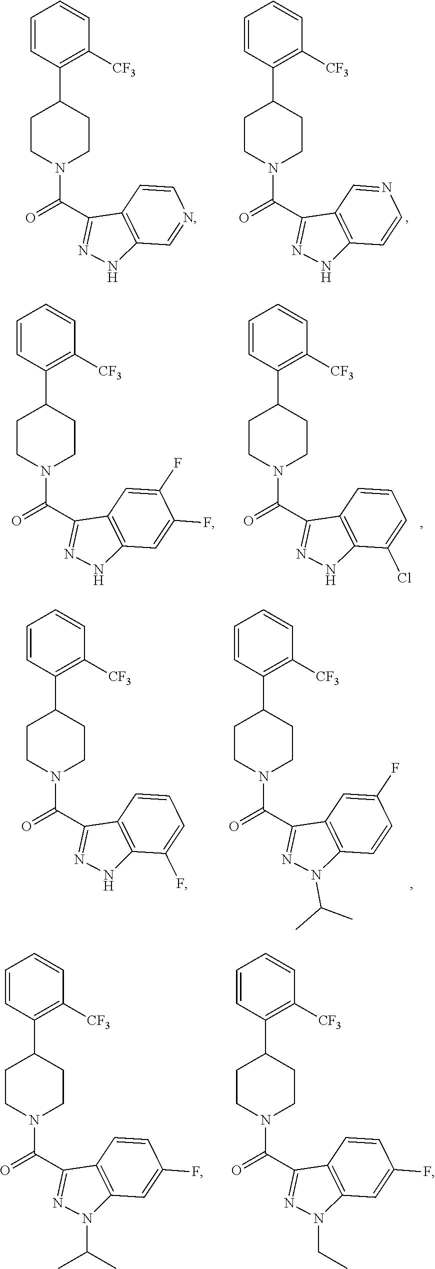 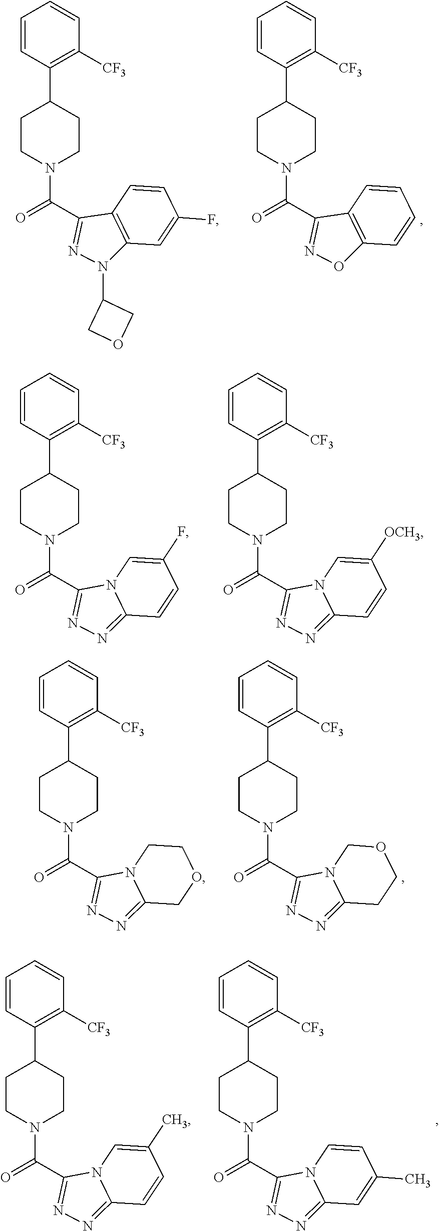 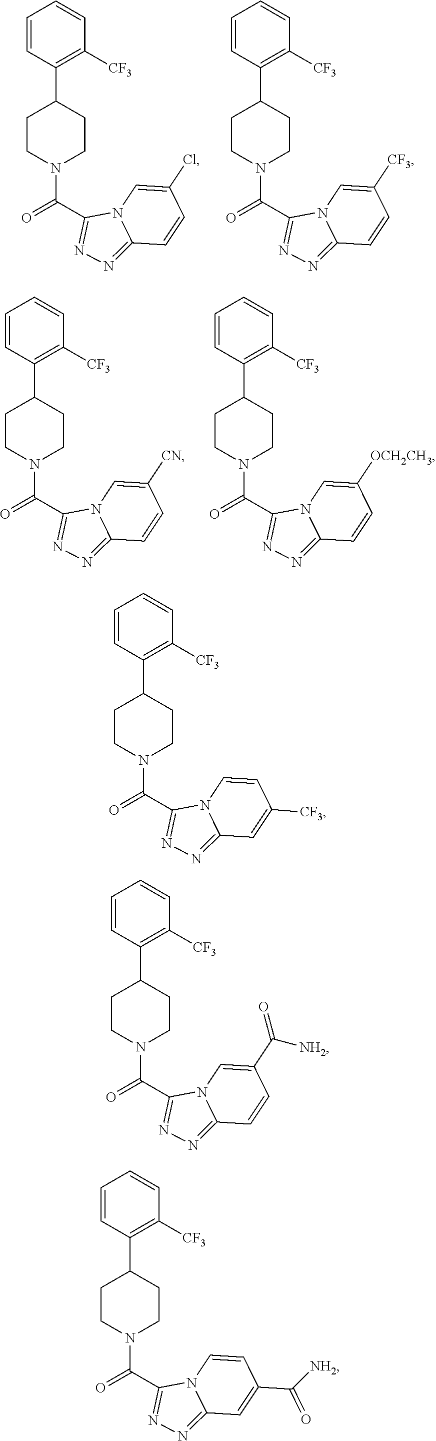 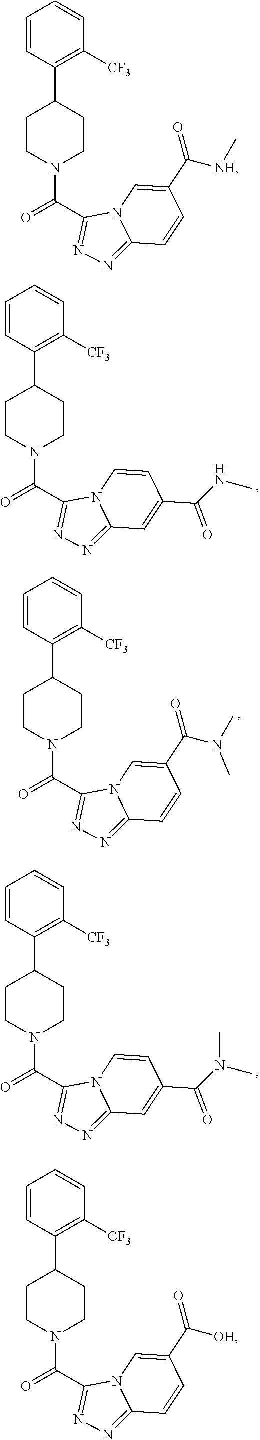 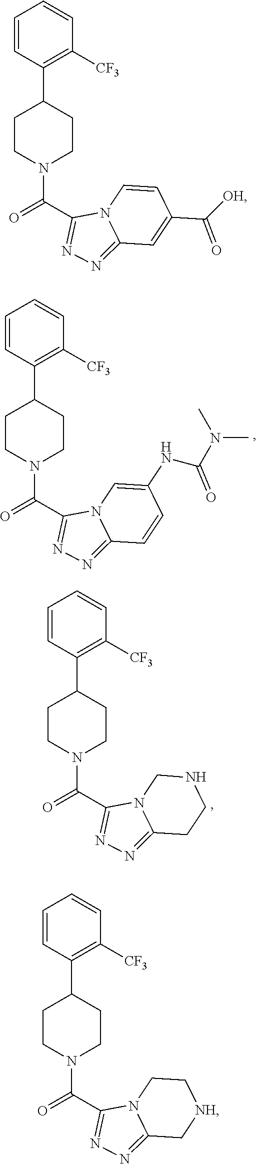 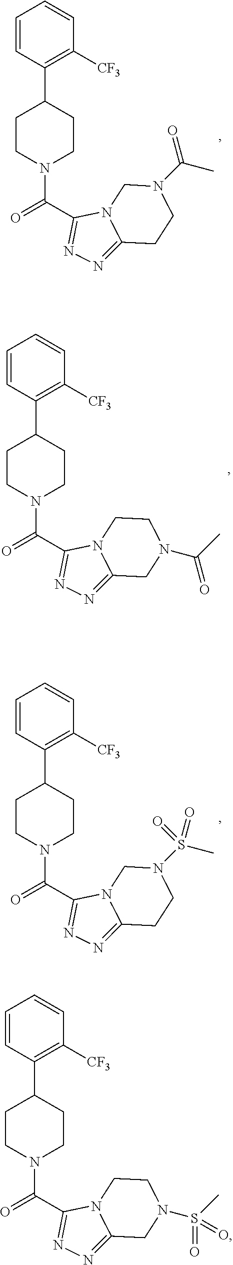 [SMILES: CC(=O)N1CCc2nnc(C(=O)N3CCC(c4ccccc4C(F)(F)F)CC3)n2C1 ; CC(=O)N1CCn2c(nnc2C(=O)N2CCC(c3ccccc3C(F)(F)F)CC2)C1 ; CS(=O)(=O)N1CCc2nnc(C(=O)N3CCC(c4ccccc4C(F)(F)F)CC3)n2C1 ; CS(=O)(=O)N1CCn2c(nnc2C(=O)N2CCC(c3ccccc3C(F)(F)F)CC2)C1] 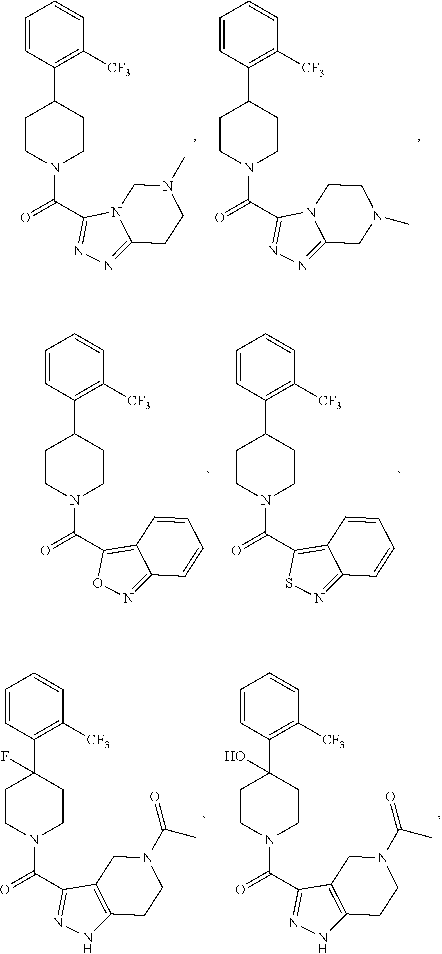 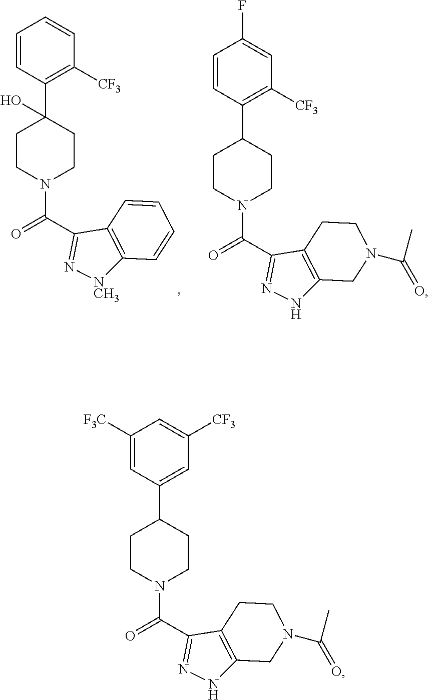 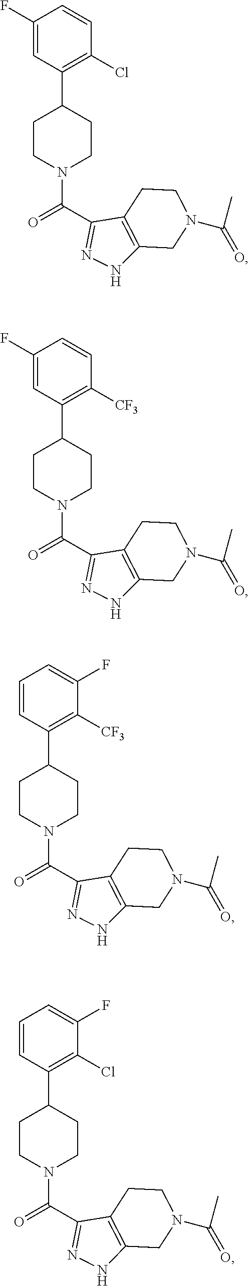 [SMILES: CC(=O)N1CCc2c(C(=O)N3CCC(c4cc(F)ccc4Cl)CC3)n[nH]c2C1 ; CC(=O)N1CCc2c(C(=O)N3CCC(c4cc(F)ccc4C(F)(F)F)CC3)n[nH]c2C1 ; CC(=O)N1CCc2c(C(=O)N3CCC(c4cccc(F)c4C(F)(F)F)CC3)n[nH]c2C1 ; CC(=O)N1CCc2c(C(=O)N3CCC(c4cccc(F)c4Cl)CC3)n[nH]c2C1] 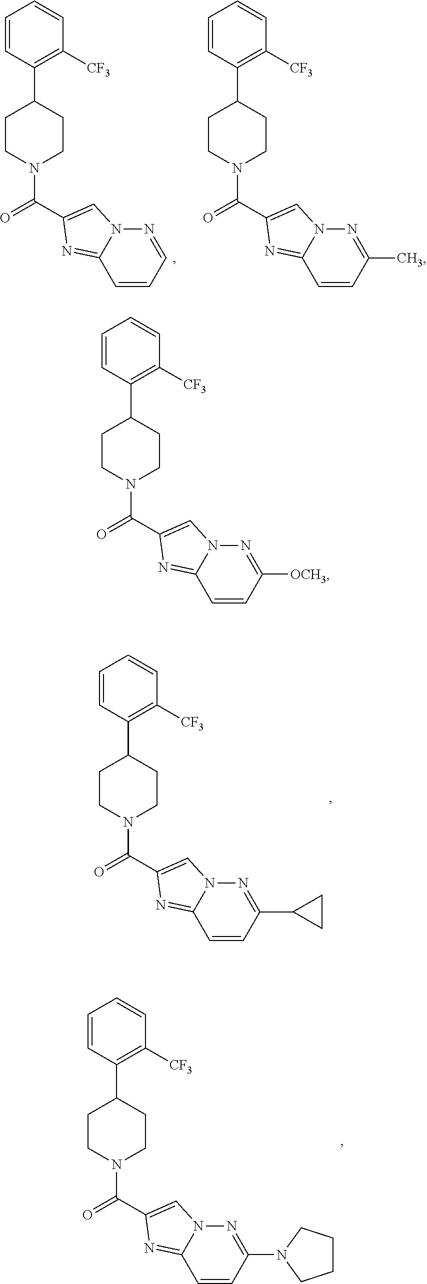 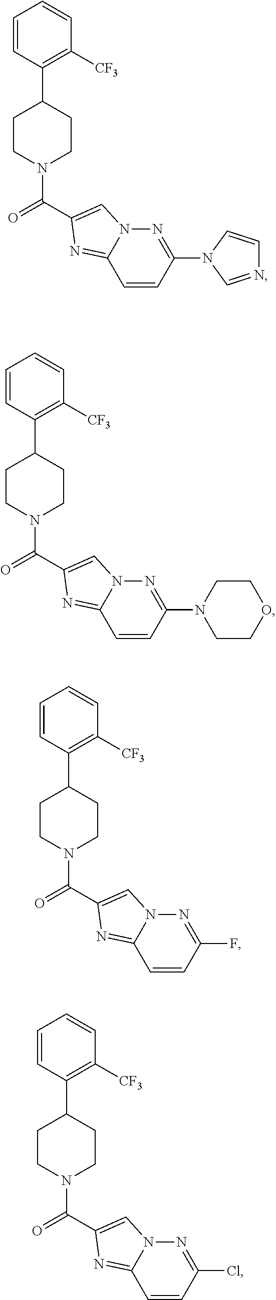 [SMILES: O=C(c1cn2nc(-n3ccnc3)ccc2n1)N1CCC(c2ccccc2C(F)(F)F)CC1 ; O=C(c1cn2nc(N3CCOCC3)ccc2n1)N1CCC(c2ccccc2C(F)(F)F)CC1 ; O=C(c1cn2nc(F)ccc2n1)N1CCC(c2ccccc2C(F)(F)F)CC1 ; O=C(c1cn2nc(Cl)ccc2n1)N1CCC(c2ccccc2C(F)(F)F)CC1] 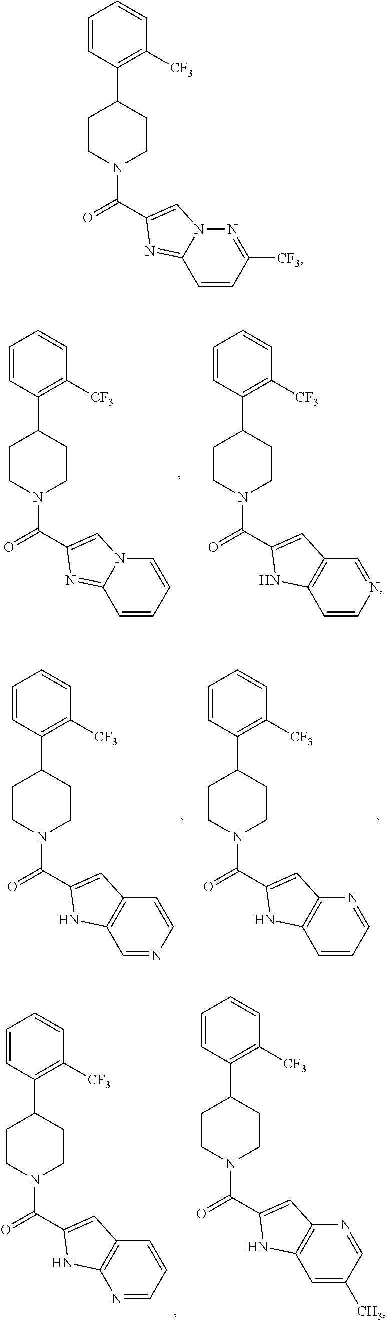 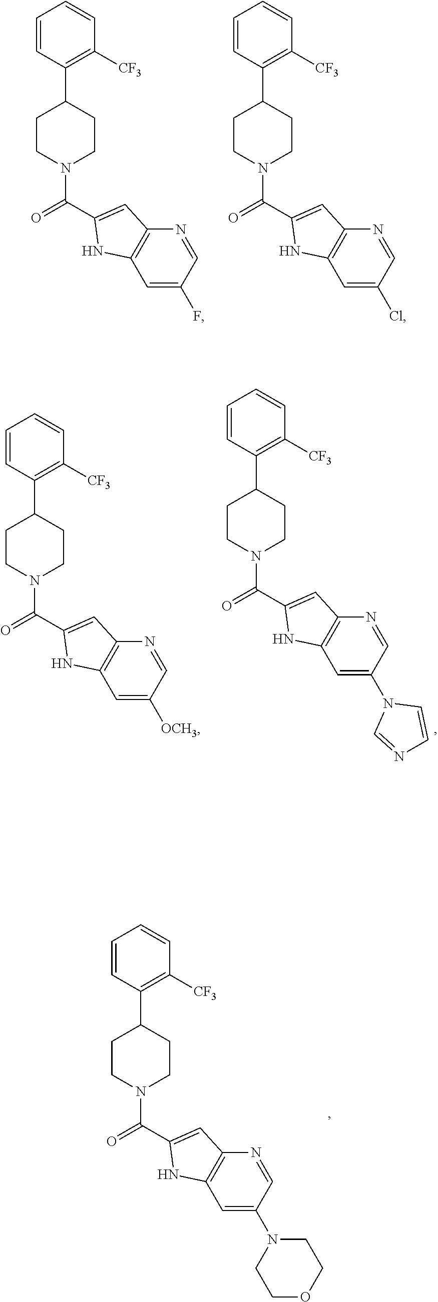 [SMILES: O=C(c1cc2ncc(F)cc2[nH]1)N1CCC(c2ccccc2C(F)(F)F)CC1 ; O=C(c1cc2ncc(Cl)cc2[nH]1)N1CCC(c2ccccc2C(F)(F)F)CC1 ; COc1cnc2cc(C(=O)N3CCC(c4ccccc4C(F)(F)F)CC3)[nH]c2c1 ; O=C(c1cc2ncc(-n3ccnc3)cc2[nH]1)N1CCC(c2ccccc2C(F)(F)F)CC1 ; O=C(c1cc2ncc(N3CCOCC3)cc2[nH]1)N1CCC(c2ccccc2C(F)(F)F)CC1] 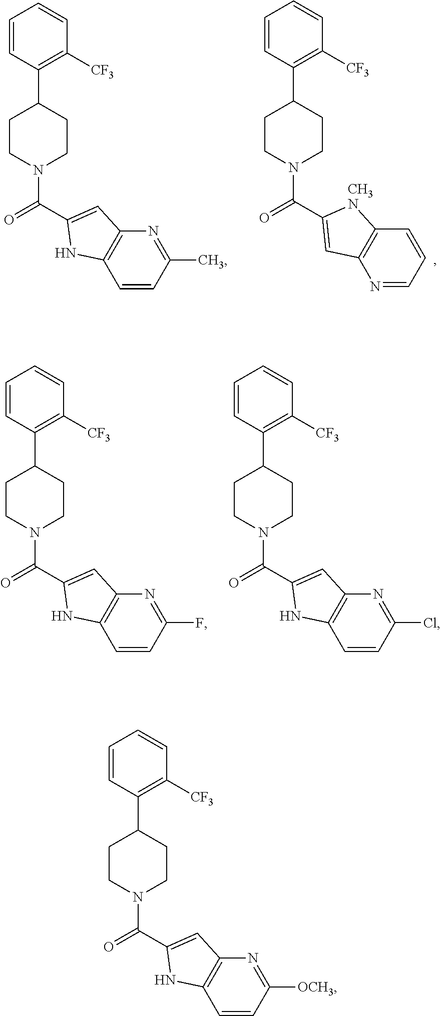 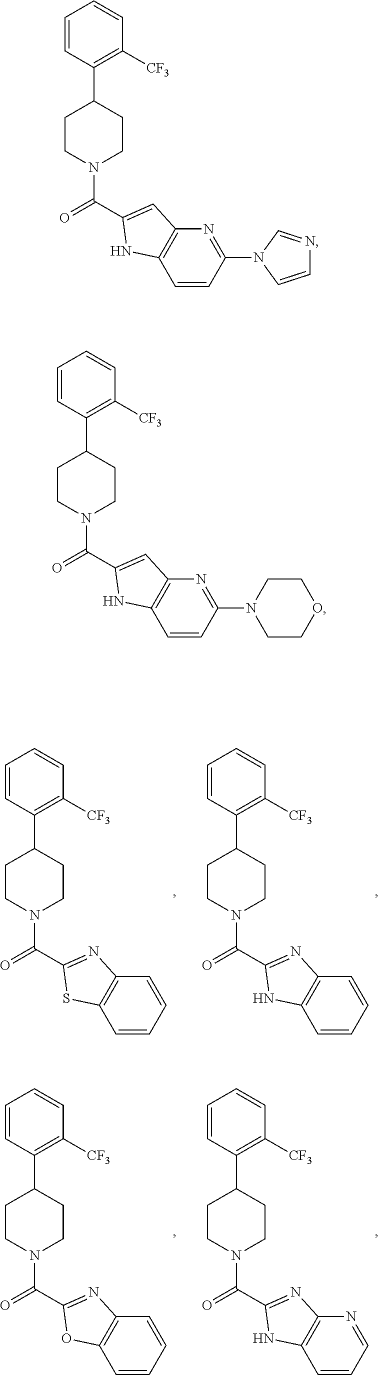 [SMILES: O=C(c1cc2nc(-n3ccnc3)ccc2[nH]1)N1CCC(c2ccccc2C(F)(F)F)CC1 ; O=C(c1cc2nc(N3CCOCC3)ccc2[nH]1)N1CCC(c2ccccc2C(F)(F)F)CC1 ; O=C(c1nc2ccccc2s1)N1CCC(c2ccccc2C(F)(F)F)CC1 ; O=C(c1nc2ccccc2[nH]1)N1CCC(c2ccccc2C(F)(F)F)CC1 ; O=C(c1nc2ccccc2o1)N1CCC(c2ccccc2C(F)(F)F)CC1 ; O=C(c1nc2ncccc2[nH]1)N1CCC(c2ccccc2C(F)(F)F)CC1] 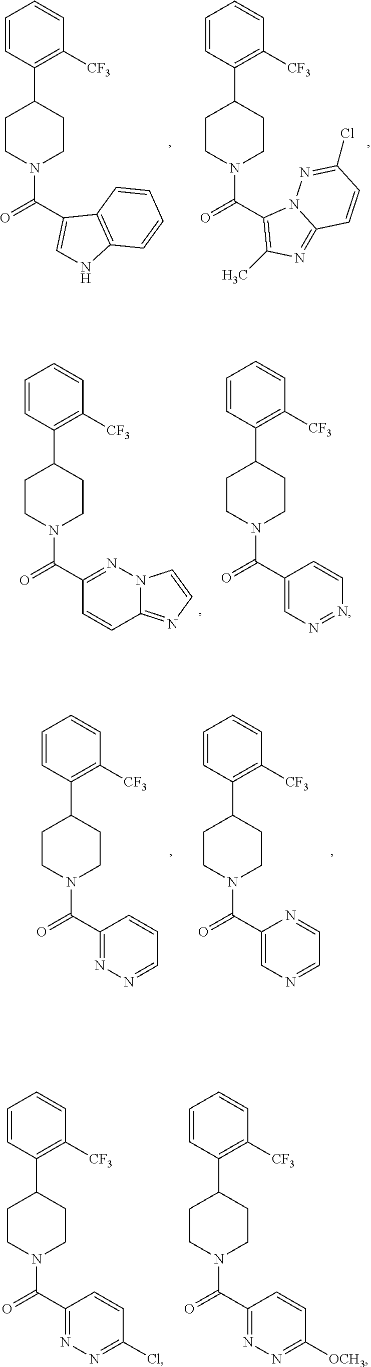 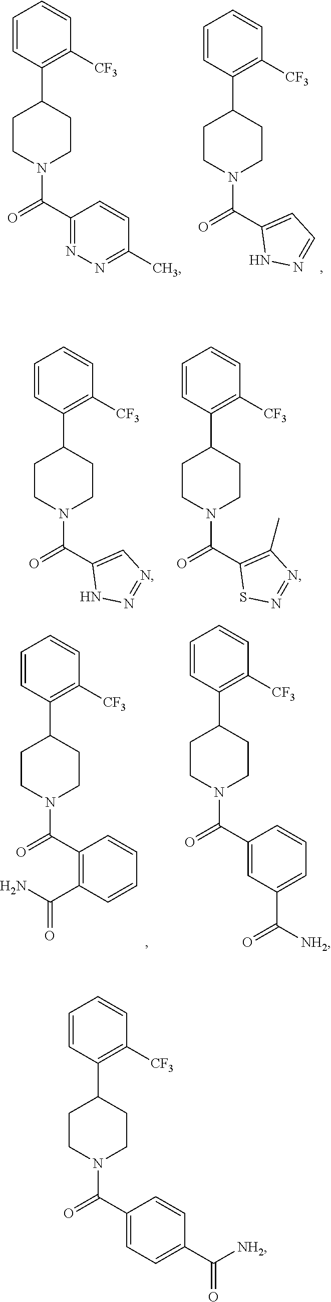 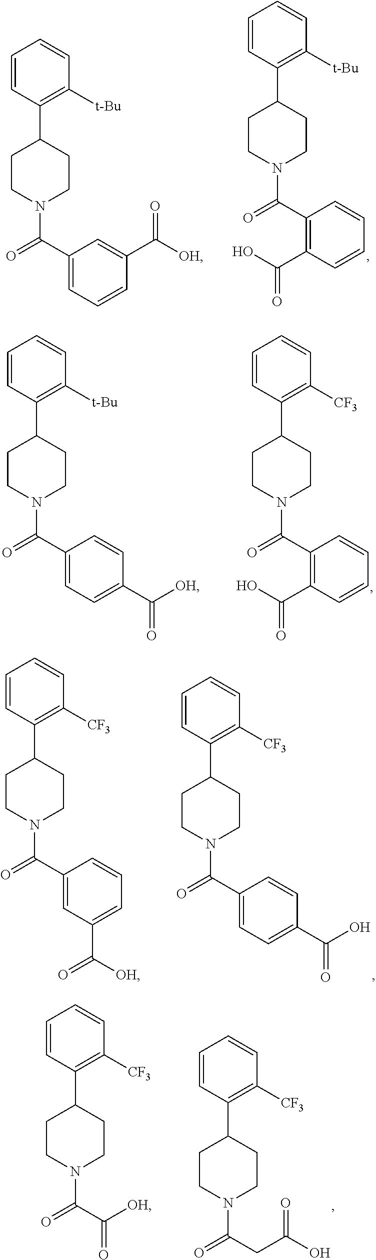 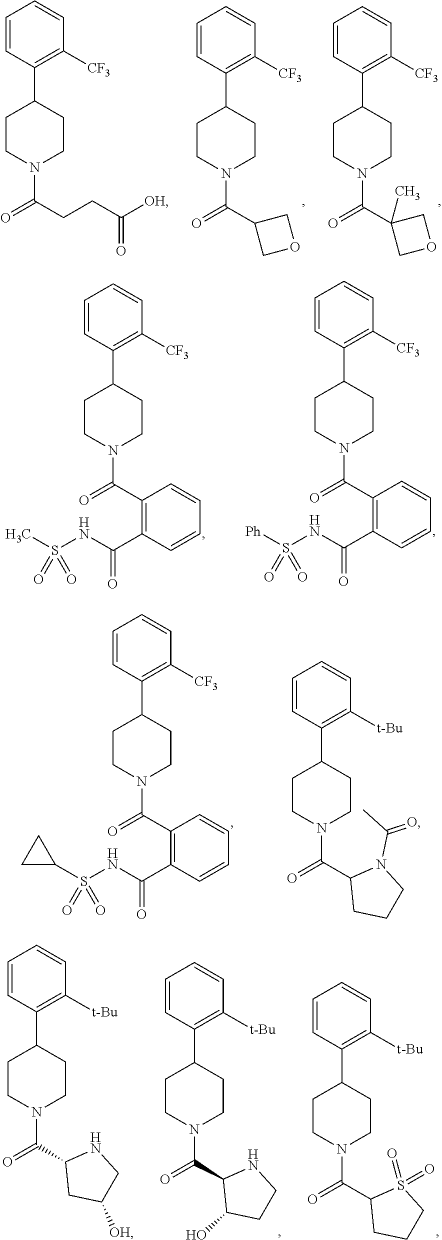 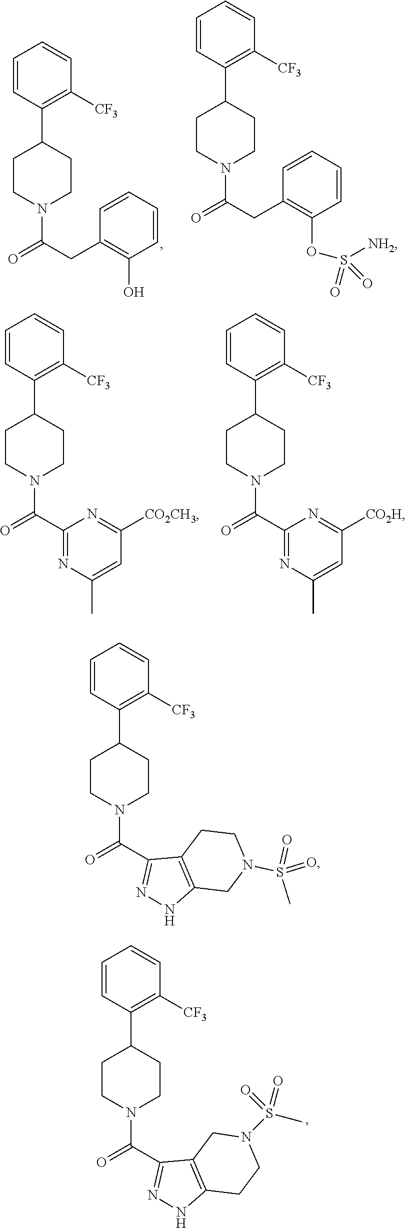 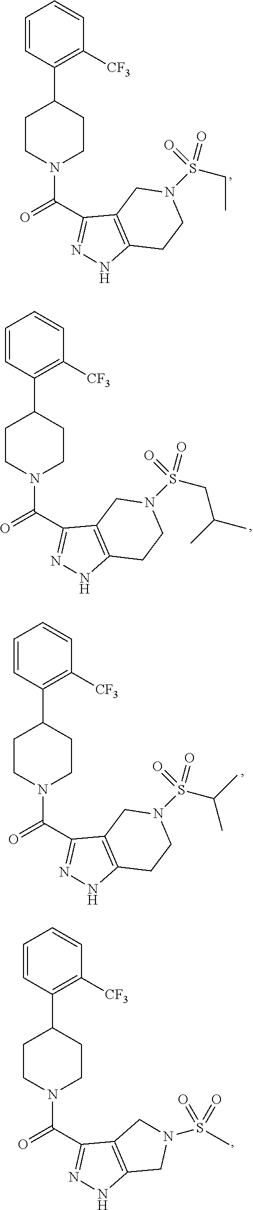 [SMILES: CCS(=O)(=O)N1CCc2[nH]nc(C(=O)N3CCC(c4ccccc4C(F)(F)F)CC3)c2C1 ; CC(C)CS(=O)(=O)N1CCc2[nH]nc(C(=O)N3CCC(c4ccccc4C(F)(F)F)CC3)c2C1 ; CC(C)S(=O)(=O)N1CCc2[nH]nc(C(=O)N3CCC(c4ccccc4C(F)(F)F)CC3)c2C1 ; CS(=O)(=O)N1Cc2[nH]nc(C(=O)N3CCC(c4ccccc4C(F)(F)F)CC3)c2C1] 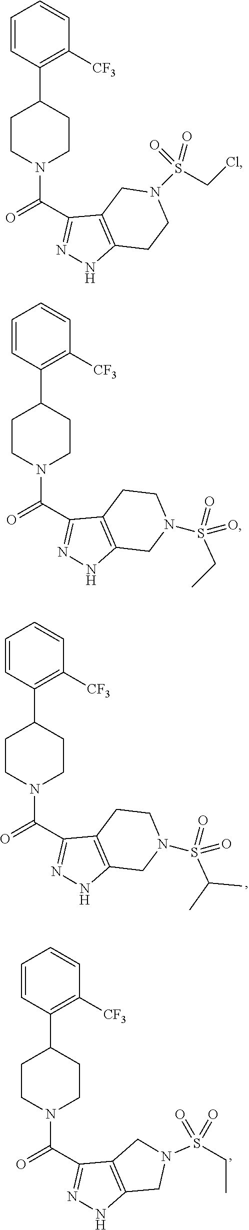 [SMILES: O=C(c1n[nH]c2c1CN(S(=O)(=O)CCl)CC2)N1CCC(c2ccccc2C(F)(F)F)CC1 ; CCS(=O)(=O)N1CCc2c(C(=O)N3CCC(c4ccccc4C(F)(F)F)CC3)n[nH]c2C1 ; CC(C)S(=O)(=O)N1CCc2c(C(=O)N3CCC(c4ccccc4C(F)(F)F)CC3)n[nH]c2C1 ; CCS(=O)(=O)N1Cc2[nH]nc(C(=O)N3CCC(c4ccccc4C(F)(F)F)CC3)c2C1] 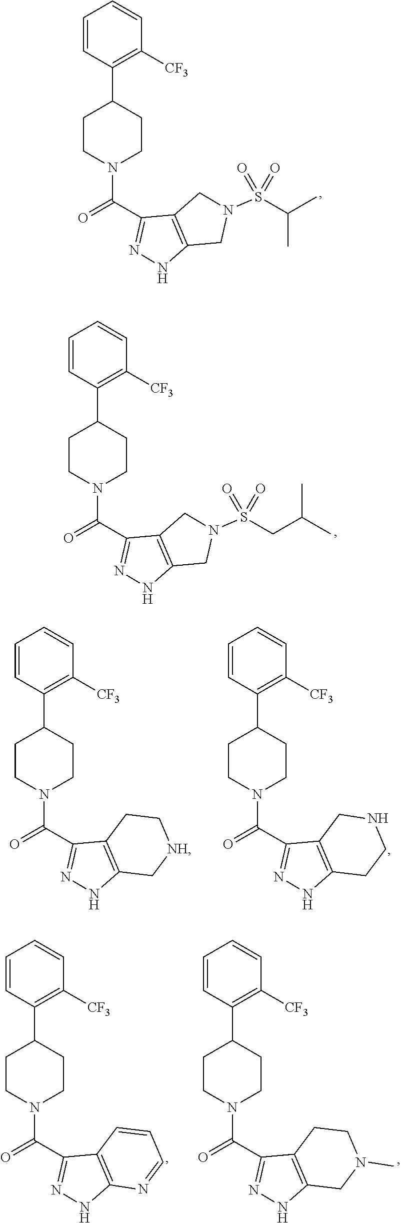 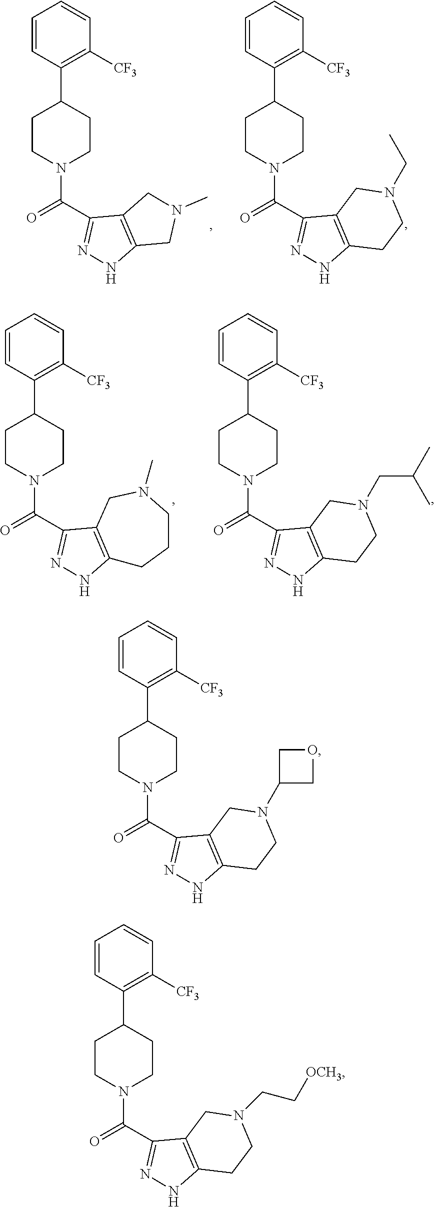 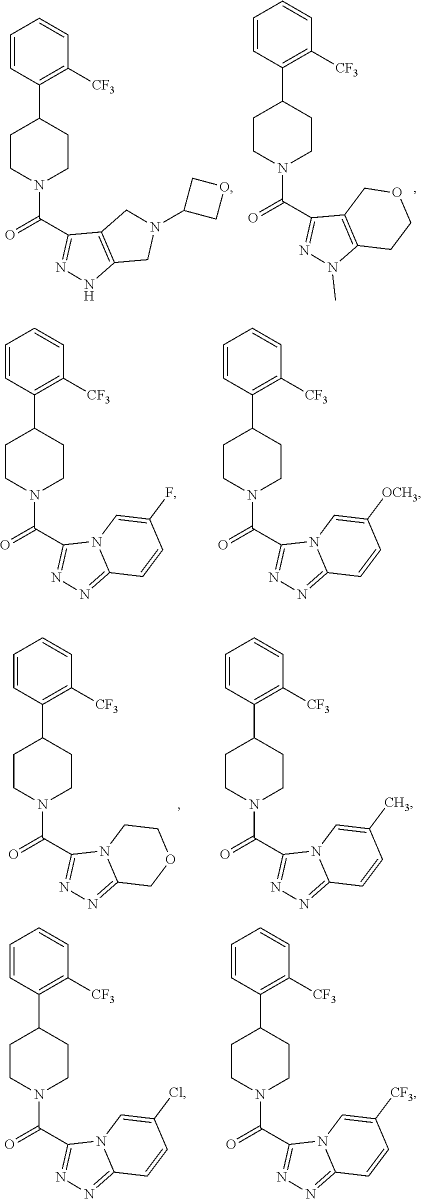 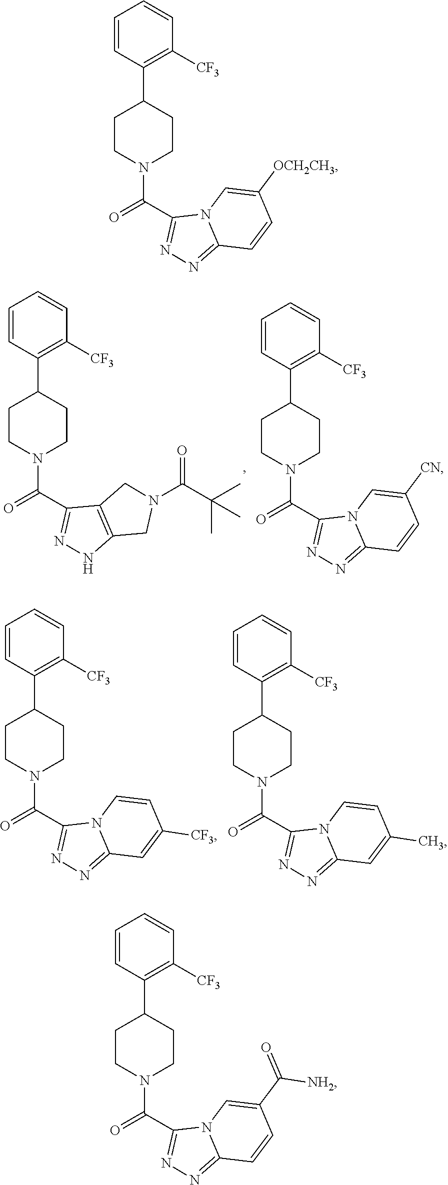 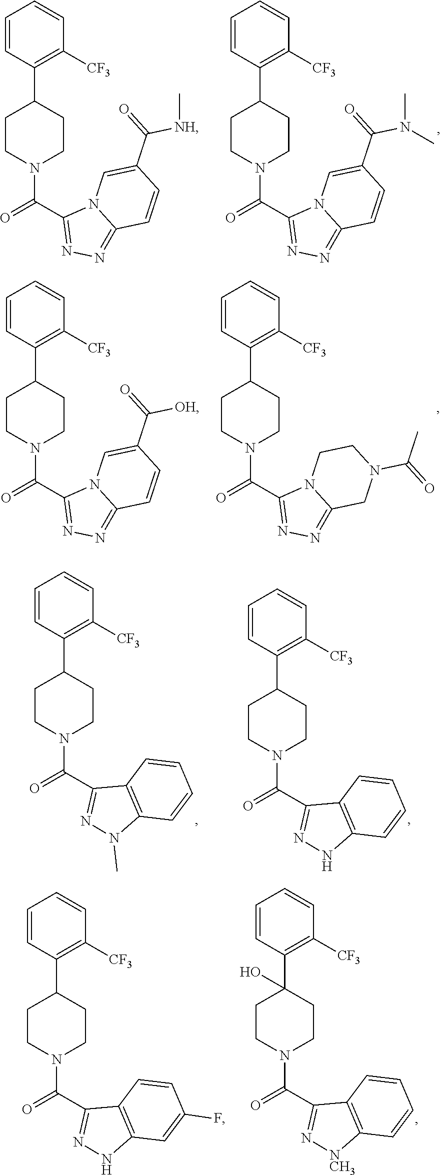 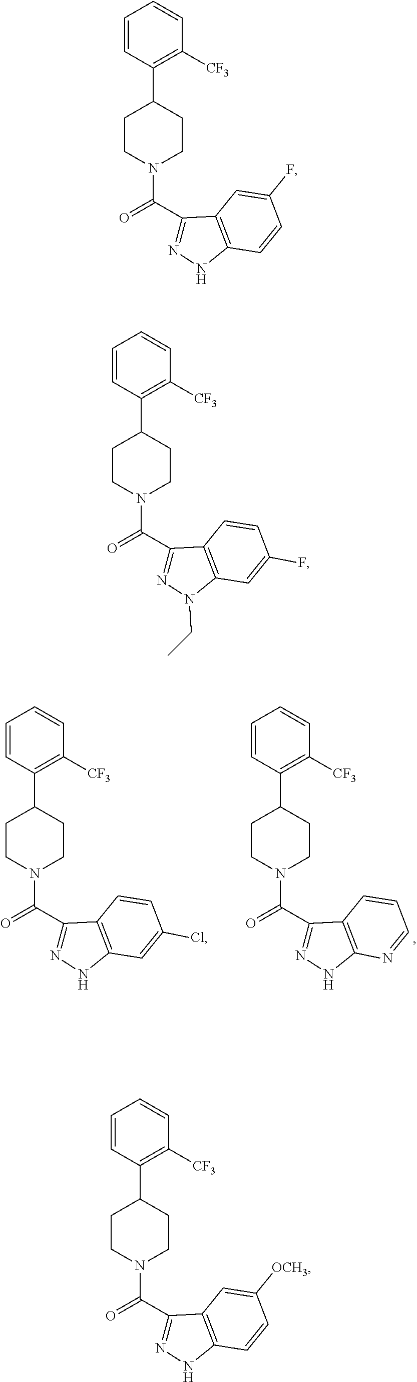 [SMILES: O=C(c1n[nH]c2ccc(F)cc12)N1CCC(c2ccccc2C(F)(F)F)CC1 ; CCn1nc(C(=O)N2CCC(c3ccccc3C(F)(F)F)CC2)c2ccc(F)cc21 ; O=C(c1n[nH]c2cc(Cl)ccc12)N1CCC(c2ccccc2C(F)(F)F)CC1 ; O=C(c1n[nH]c2ncccc12)N1CCC(c2ccccc2C(F)(F)F)CC1 ; COc1ccc2[nH]nc(C(=O)N3CCC(c4ccccc4C(F)(F)F)CC3)c2c1] 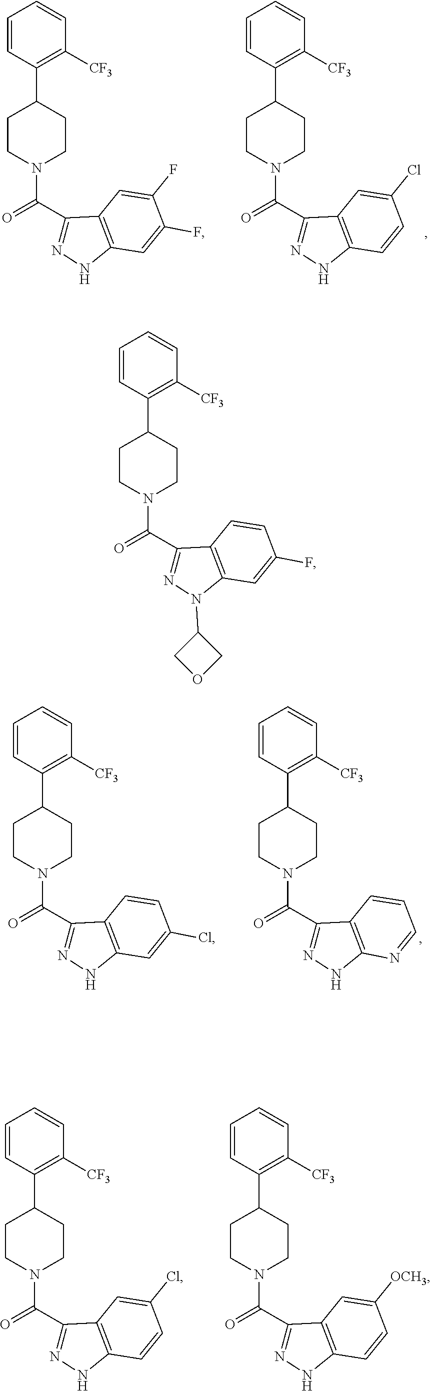 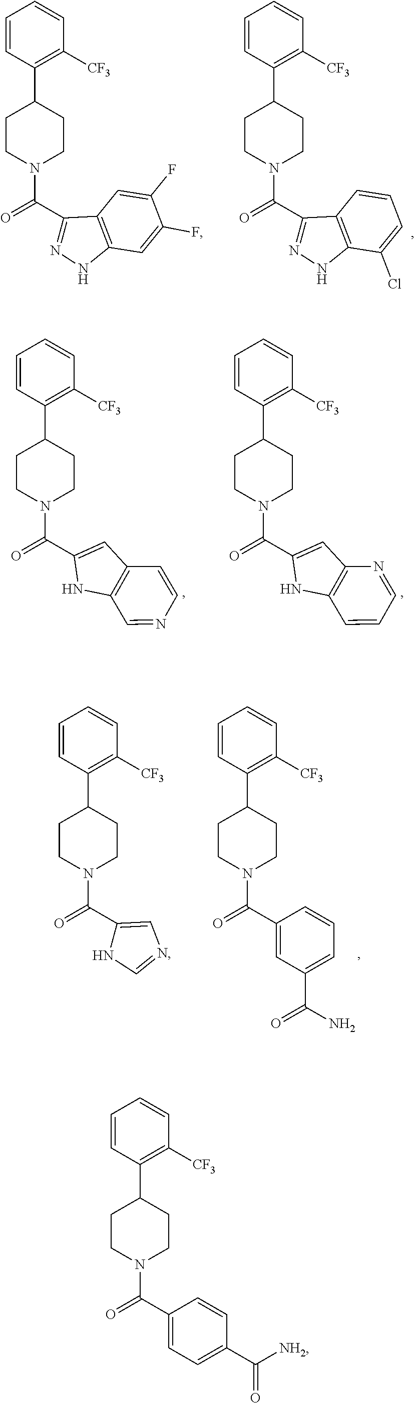 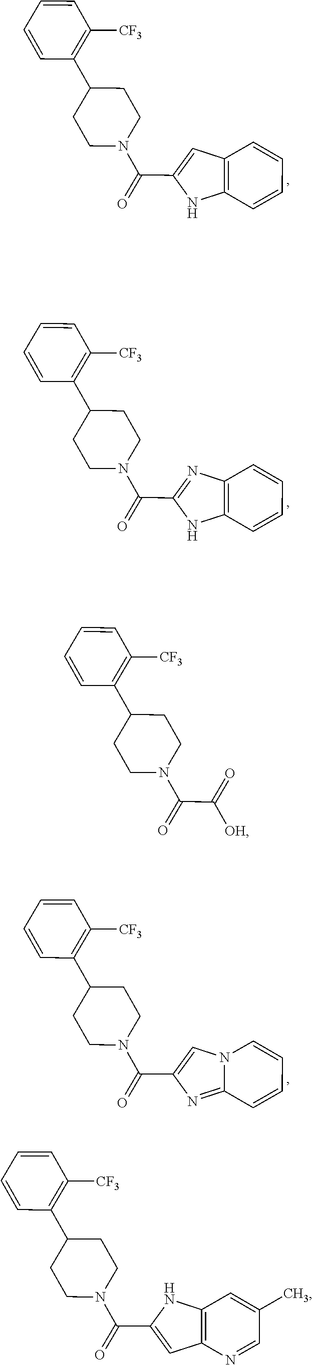 [SMILES: O=C(c1cc2ccccc2[nH]1)N1CCC(c2ccccc2C(F)(F)F)CC1 ; O=C(c1nc2ccccc2[nH]1)N1CCC(c2ccccc2C(F)(F)F)CC1 ; O=C(O)C(=O)N1CCC(c2ccccc2C(F)(F)F)CC1 ; O=C(c1cn2ccccc2n1)N1CCC(c2ccccc2C(F)(F)F)CC1 ; Cc1cnc2cc(C(=O)N3CCC(c4ccccc4C(F)(F)F)CC3)[nH]c2c1] 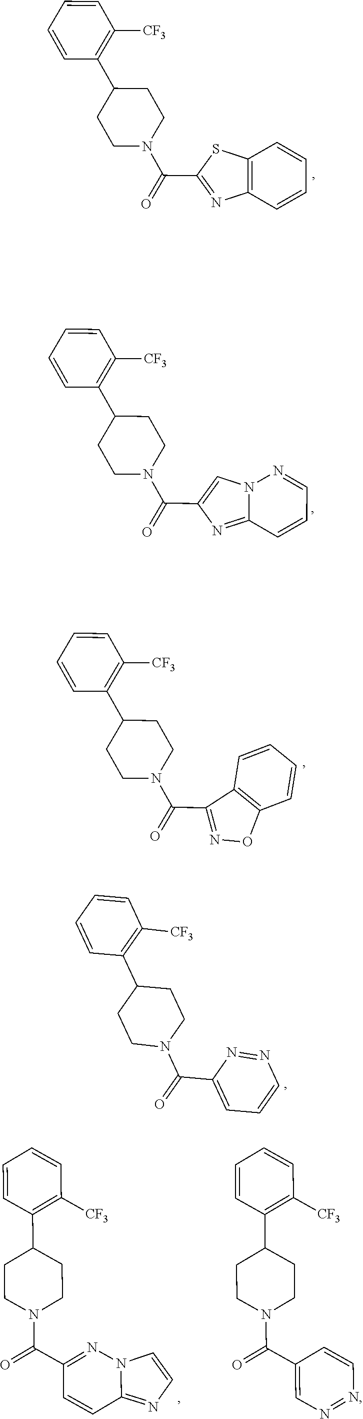 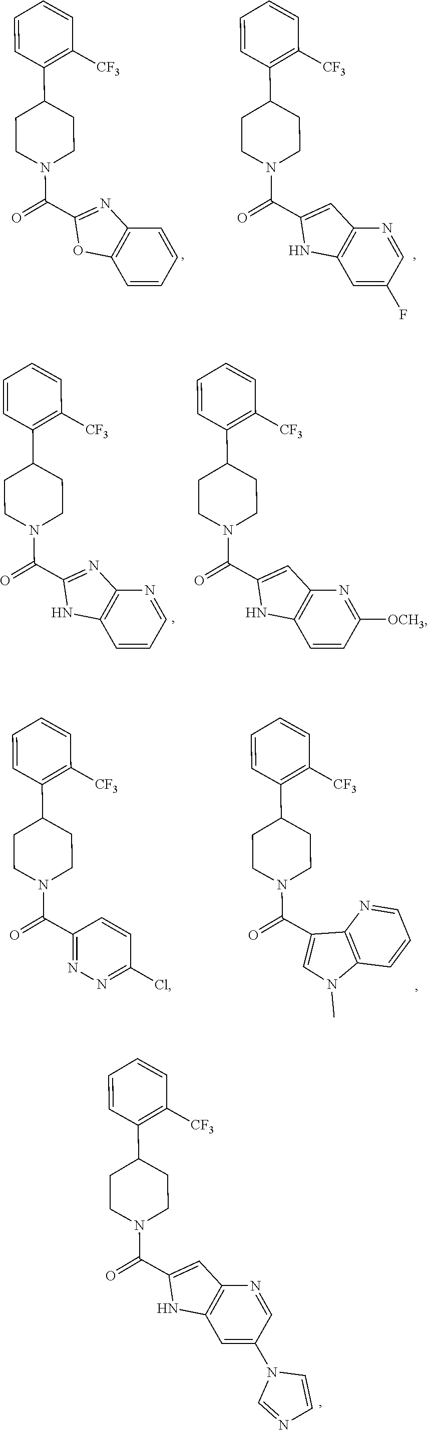 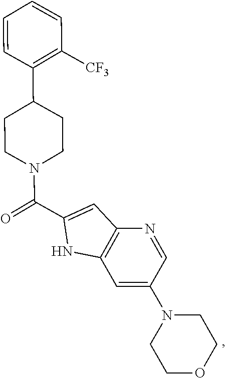 [SMILES: O=C(c1cc2ncc(N3CCOCC3)cc2[nH]1)N1CCC(c2ccccc2C(F)(F)F)CC1] 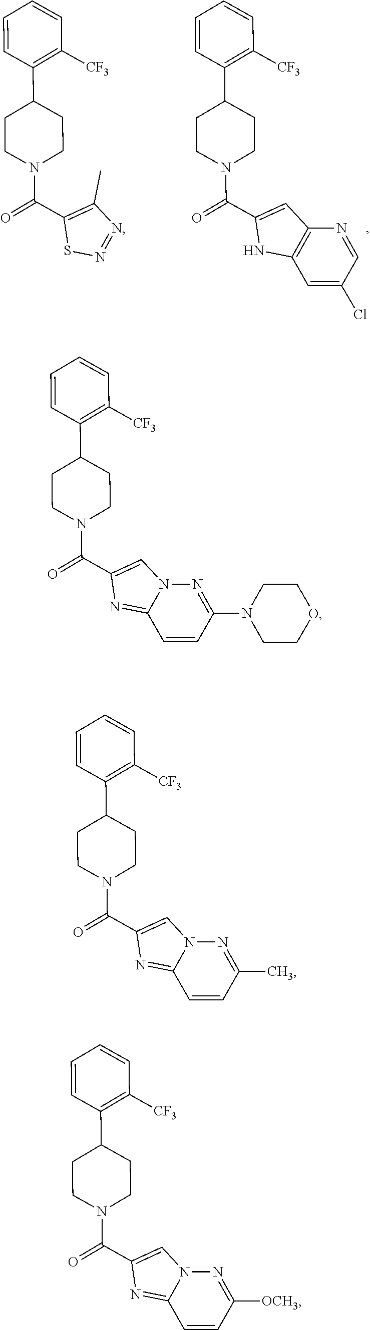 [SMILES: Cc1nnsc1C(=O)N1CCC(c2ccccc2C(F)(F)F)CC1 ; O=C(c1cc2ncc(Cl)cc2[nH]1)N1CCC(c2ccccc2C(F)(F)F)CC1 ; O=C(c1cn2nc(N3CCOCC3)ccc2n1)N1CCC(c2ccccc2C(F)(F)F)CC1 ; Cc1ccc2nc(C(=O)N3CCC(c4ccccc4C(F)(F)F)CC3)cn2n1 ; COc1ccc2nc(C(=O)N3CCC(c4ccccc4C(F)(F)F)CC3)cn2n1] 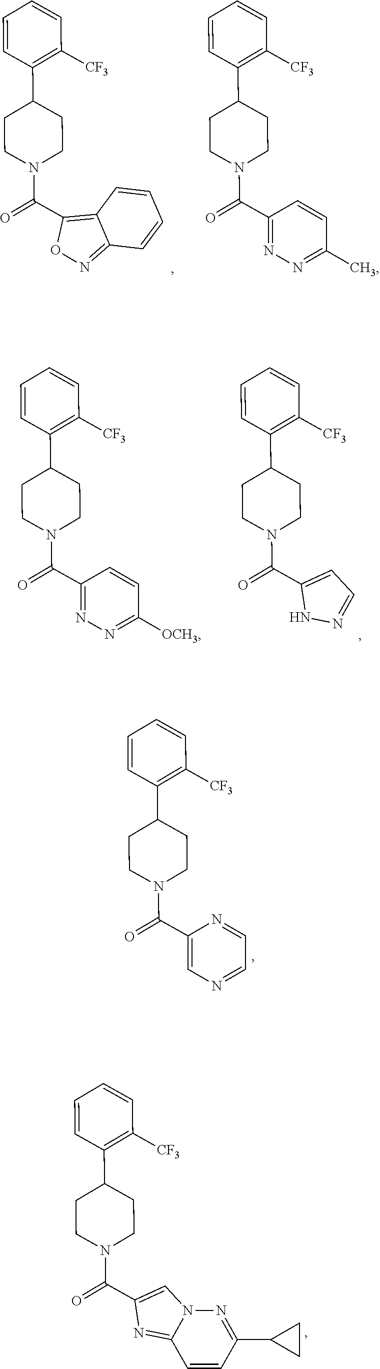 [SMILES: O=C(c1onc2ccccc12)N1CCC(c2ccccc2C(F)(F)F)CC1 ; Cc1ccc(C(=O)N2CCC(c3ccccc3C(F)(F)F)CC2)nn1 ; COc1ccc(C(=O)N2CCC(c3ccccc3C(F)(F)F)CC2)nn1 ; O=C(c1ccn[nH]1)N1CCC(c2ccccc2C(F)(F)F)CC1 ; O=C(c1cnccn1)N1CCC(c2ccccc2C(F)(F)F)CC1 ; O=C(c1cn2nc(C3CC3)ccc2n1)N1CCC(c2ccccc2C(F)(F)F)CC1] 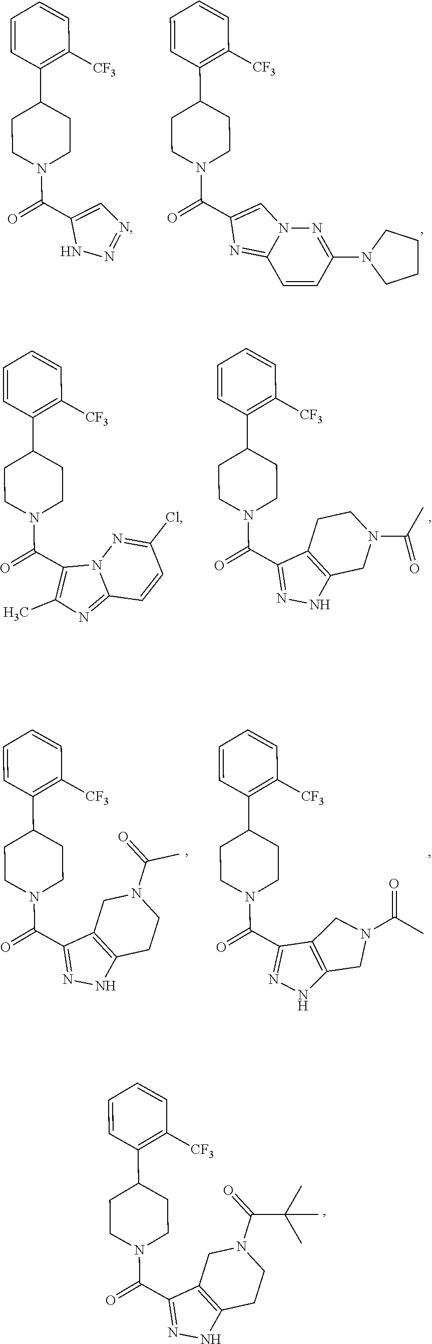 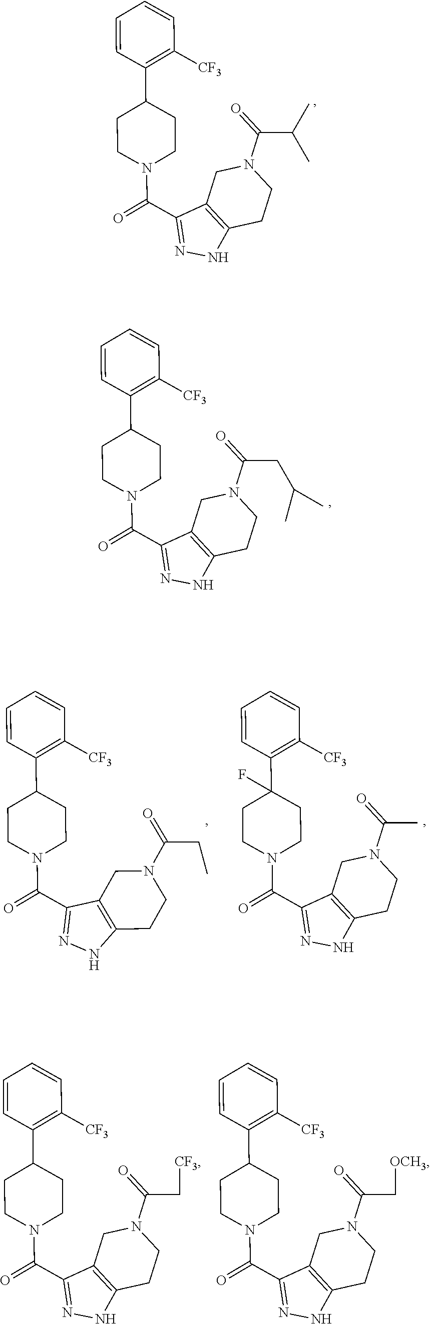 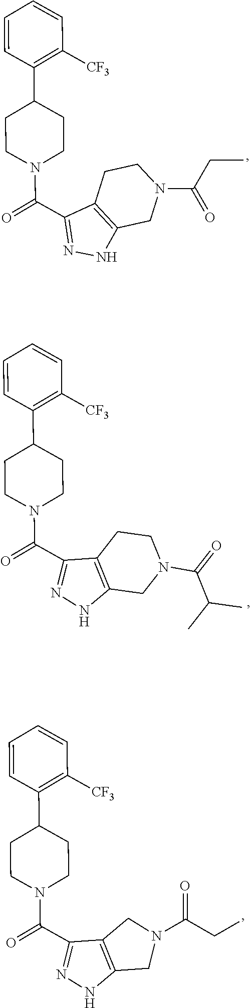 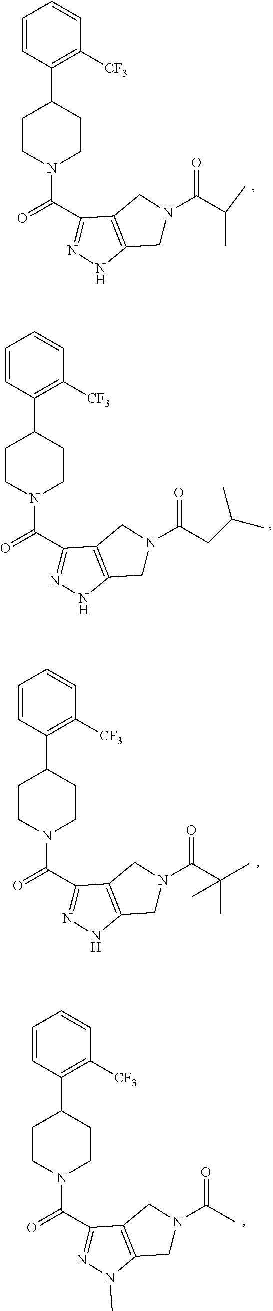 [SMILES: CC(C)C(=O)N1Cc2[nH]nc(C(=O)N3CCC(c4ccccc4C(F)(F)F)CC3)c2C1 ; CC(C)CC(=O)N1Cc2[nH]nc(C(=O)N3CCC(c4ccccc4C(F)(F)F)CC3)c2C1 ; CC(C)(C)C(=O)N1Cc2[nH]nc(C(=O)N3CCC(c4ccccc4C(F)(F)F)CC3)c2C1 ; CC(=O)N1Cc2c(C(=O)N3CCC(c4ccccc4C(F)(F)F)CC3)nn(C)c2C1] 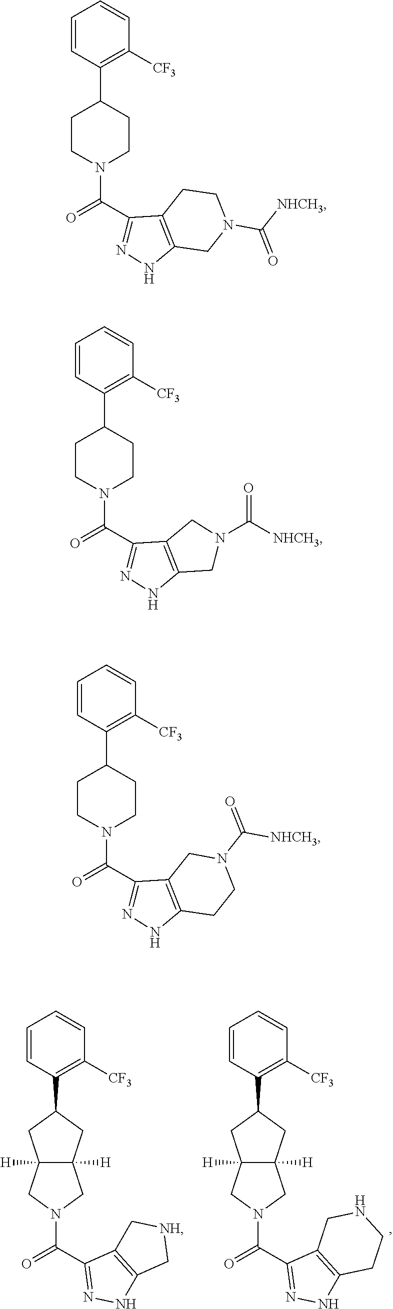 [SMILES: CNC(=O)N1CCc2c(C(=O)N3CCC(c4ccccc4C(F)(F)F)CC3)n[nH]c2C1 ; CNC(=O)N1Cc2[nH]nc(C(=O)N3CCC(c4ccccc4C(F)(F)F)CC3)c2C1 ; CNC(=O)N1CCc2[nH]nc(C(=O)N3CCC(c4ccccc4C(F)(F)F)CC3)c2C1 ; O=C(c1n[nH]c2c1CNC2)N1C[C@H]2C[C@@H](c3ccccc3C(F)(F)F)C[C@H]2C1 ; O=C(c1n[nH]c2c1CNCC2)N1C[C@H]2C[C@@H](c3ccccc3C(F)(F)F)C[C@H]2C1] 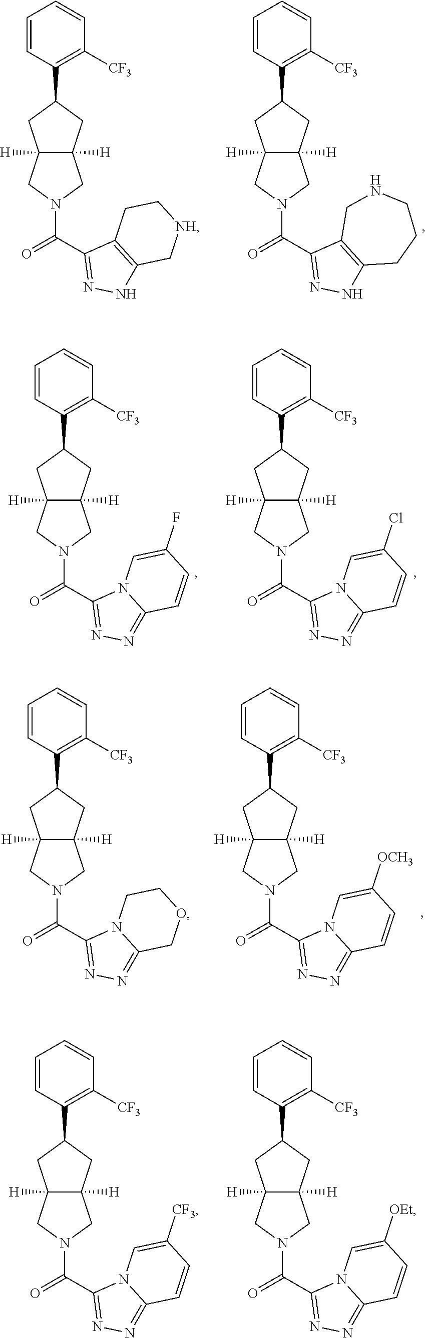 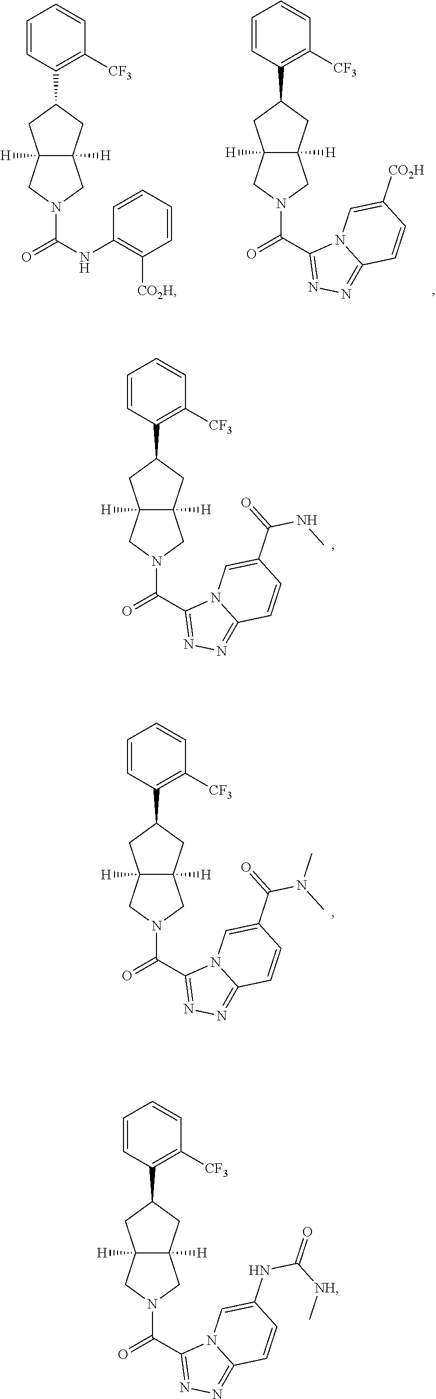 [SMILES: O=C(O)c1ccccc1NC(=O)N1C[C@H]2C[C@H](c3ccccc3C(F)(F)F)C[C@H]2C1 ; O=C(O)c1ccc2nnc(C(=O)N3C[C@H]4C[C@@H](c5ccccc5C(F)(F)F)C[C@H]4C3)n2c1 ; CNC(=O)c1ccc2nnc(C(=O)N3C[C@H]4C[C@@H](c5ccccc5C(F)(F)F)C[C@H]4C3)n2c1 ; CN(C)C(=O)c1ccc2nnc(C(=O)N3C[C@H]4C[C@@H](c5ccccc5C(F)(F)F)C[C@H]4C3)n2c1 ; CNC(=O)Nc1ccc2nnc(C(=O)N3C[C@H]4C[C@@H](c5ccccc5C(F)(F)F)C[C@H]4C3)n2c1] 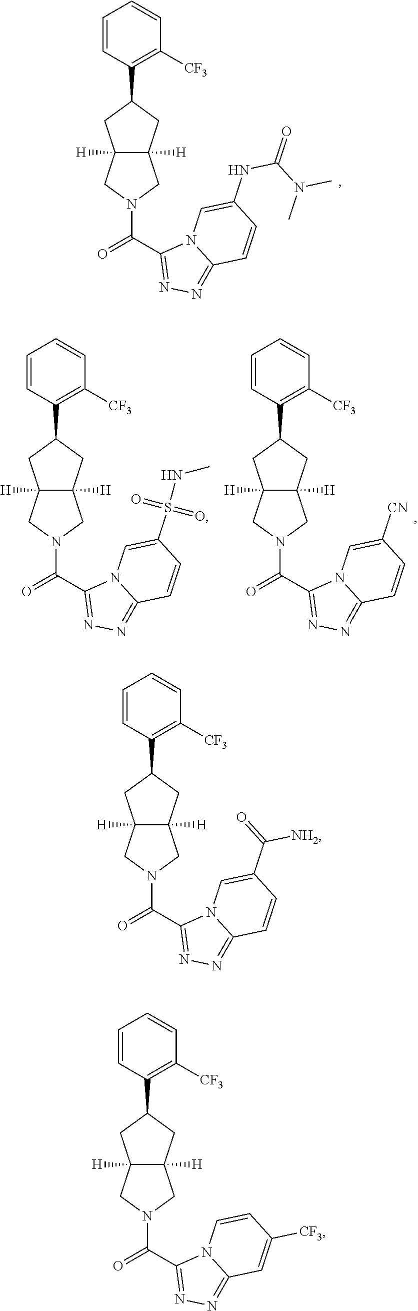 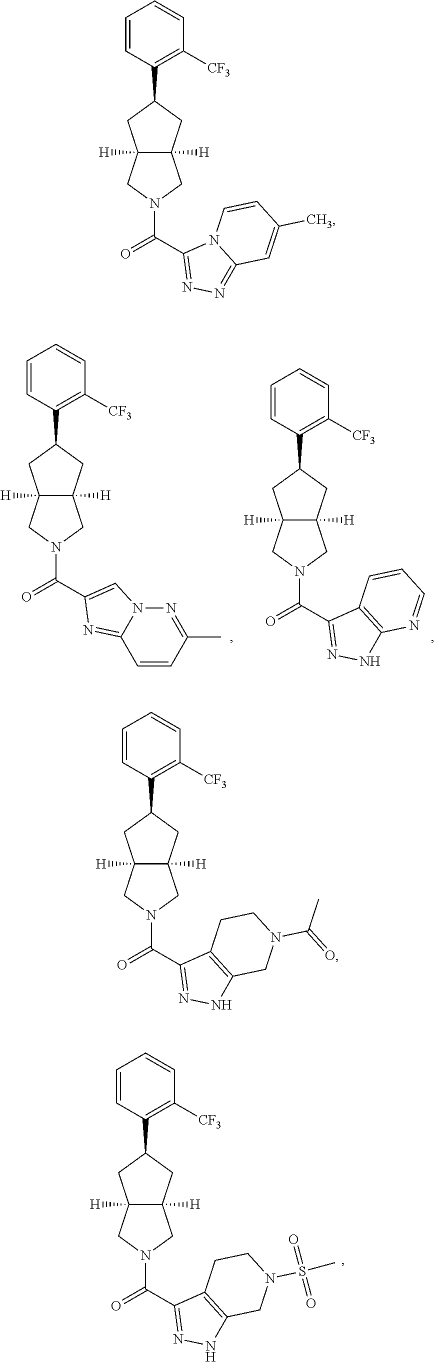 [SMILES: Cc1ccn2c(C(=O)N3C[C@H]4C[C@@H](c5ccccc5C(F)(F)F)C[C@H]4C3)nnc2c1 ; Cc1ccc2nc(C(=O)N3C[C@H]4C[C@@H](c5ccccc5C(F)(F)F)C[C@H]4C3)cn2n1 ; O=C(c1n[nH]c2ncccc12)N1C[C@H]2C[C@@H](c3ccccc3C(F)(F)F)C[C@H]2C1 ; CC(=O)N1CCc2c(C(=O)N3C[C@H]4C[C@@H](c5ccccc5C(F)(F)F)C[C@H]4C3)n[nH]c2C1 ; CS(=O)(=O)N1CCc2c(C(=O)N3C[C@H]4C[C@@H](c5ccccc5C(F)(F)F)C[C@H]4C3)n[nH]c2C1] 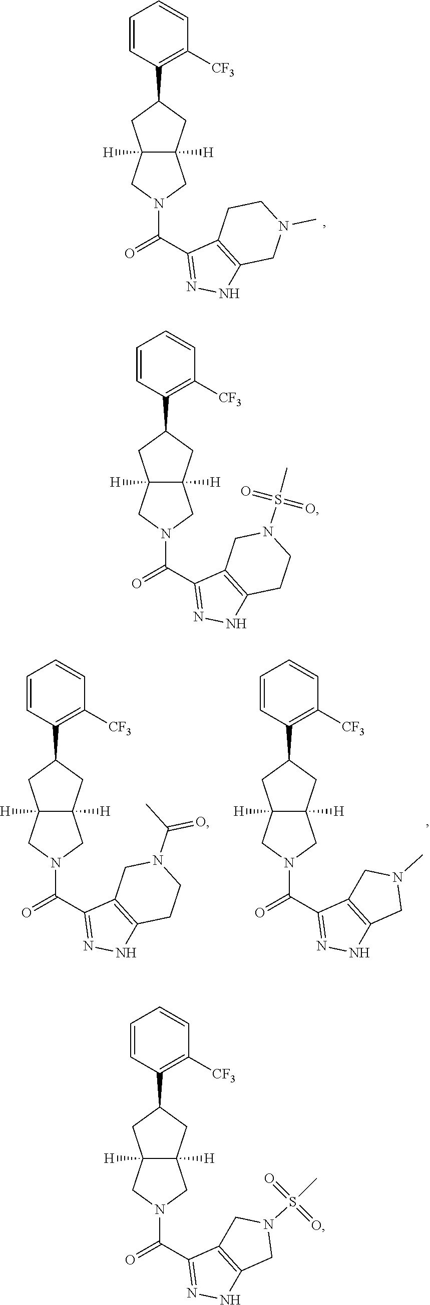 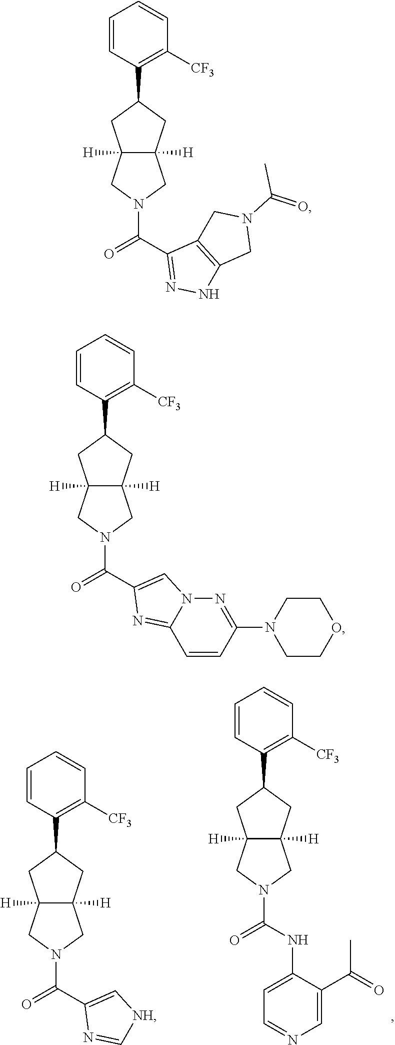 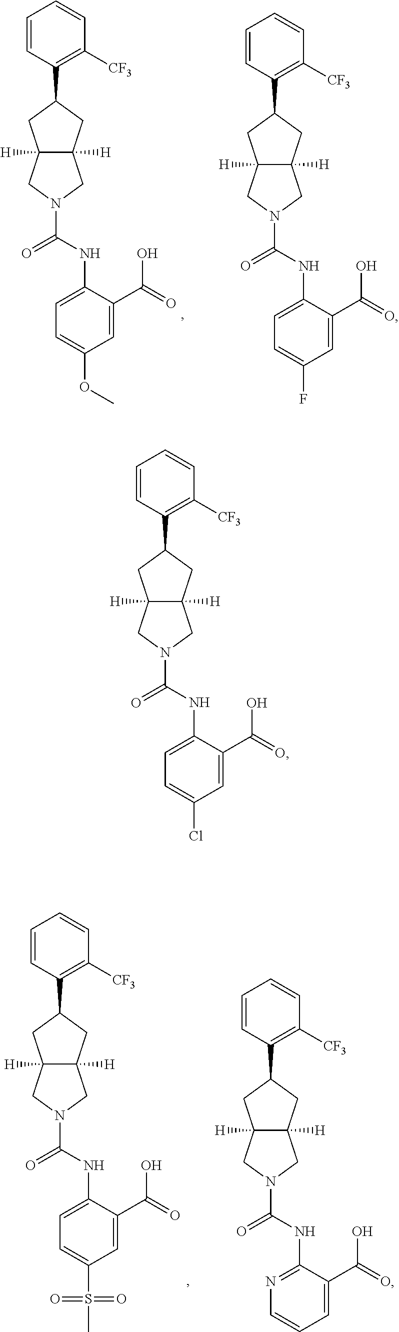 [SMILES: COc1ccc(NC(=O)N2C[C@H]3C[C@@H](c4ccccc4C(F)(F)F)C[C@H]3C2)c(C(=O)O)c1 ; O=C(O)c1cc(F)ccc1NC(=O)N1C[C@H]2C[C@@H](c3ccccc3C(F)(F)F)C[C@H]2C1 ; O=C(O)c1cc(Cl)ccc1NC(=O)N1C[C@H]2C[C@@H](c3ccccc3C(F)(F)F)C[C@H]2C1 ; CS(=O)(=O)c1ccc(NC(=O)N2C[C@H]3C[C@@H](c4ccccc4C(F)(F)F)C[C@H]3C2)c(C(=O)O)c1 ; O=C(O)c1cccnc1NC(=O)N1C[C@H]2C[C@@H](c3ccccc3C(F)(F)F)C[C@H]2C1] 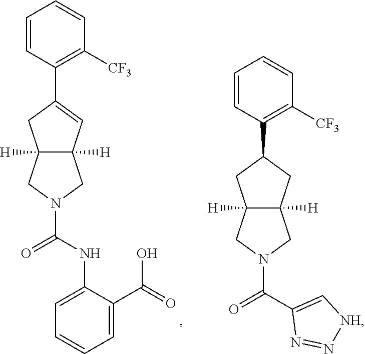 [SMILES: O=C(O)c1ccccc1NC(=O)N1C[C@H]2CC(c3ccccc3C(F)(F)F)=C[C@H]2C1 ; O=C(c1c[nH]nn1)N1C[C@H]2C[C@@H](c3ccccc3C(F)(F)F)C[C@H]2C1] 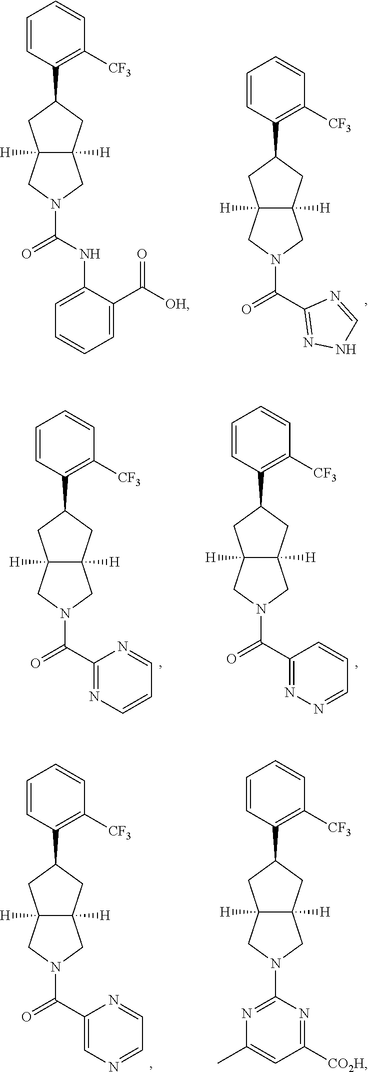 [SMILES: O=C(O)c1ccccc1NC(=O)N1C[C@H]2C[C@@H](c3ccccc3C(F)(F)F)C[C@H]2C1 ; O=C(c1nc[nH]n1)N1C[C@H]2C[C@@H](c3ccccc3C(F)(F)F)C[C@H]2C1 ; O=C(c1ncccn1)N1C[C@H]2C[C@@H](c3ccccc3C(F)(F)F)C[C@H]2C1 ; O=C(c1cccnn1)N1C[C@H]2C[C@@H](c3ccccc3C(F)(F)F)C[C@H]2C1 ; O=C(c1cnccn1)N1C[C@H]2C[C@@H](c3ccccc3C(F)(F)F)C[C@H]2C1 ; Cc1cc(C(=O)O)nc(N2C[C@H]3C[C@@H](c4ccccc4C(F)(F)F)C[C@H]3C2)n1] 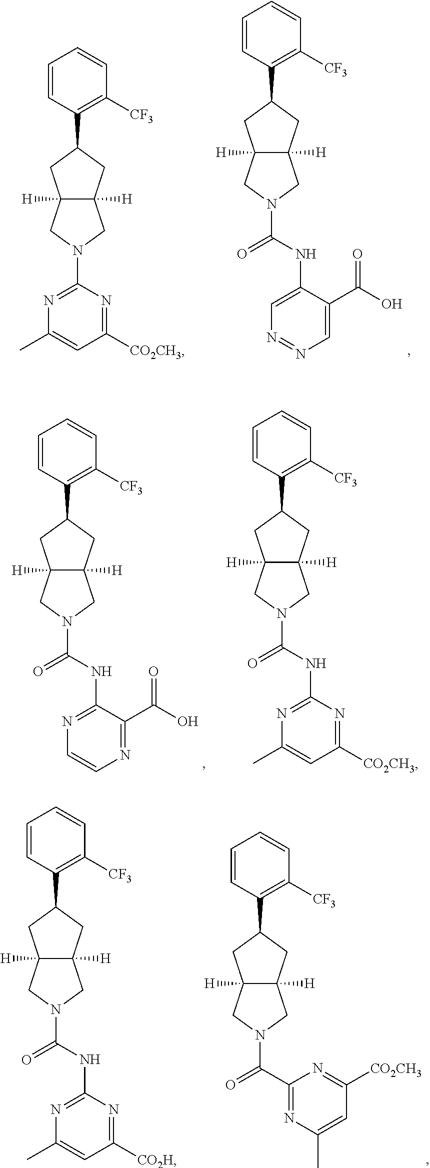 [SMILES: COC(=O)c1cc(C)nc(N2C[C@H]3C[C@@H](c4ccccc4C(F)(F)F)C[C@H]3C2)n1 ; O=C(O)c1cnncc1NC(=O)N1C[C@H]2C[C@@H](c3ccccc3C(F)(F)F)C[C@H]2C1 ; O=C(O)c1nccnc1NC(=O)N1C[C@H]2C[C@@H](c3ccccc3C(F)(F)F)C[C@H]2C1 ; COC(=O)c1cc(C)nc(NC(=O)N2C[C@H]3C[C@@H](c4ccccc4C(F)(F)F)C[C@H]3C2)n1 ; Cc1cc(C(=O)O)nc(NC(=O)N2C[C@H]3C[C@@H](c4ccccc4C(F)(F)F)C[C@H]3C2)n1 ; COC(=O)c1cc(C)nc(C(=O)N2C[C@H]3C[C@@H](c4ccccc4C(F)(F)F)C[C@H]3C2)n1] 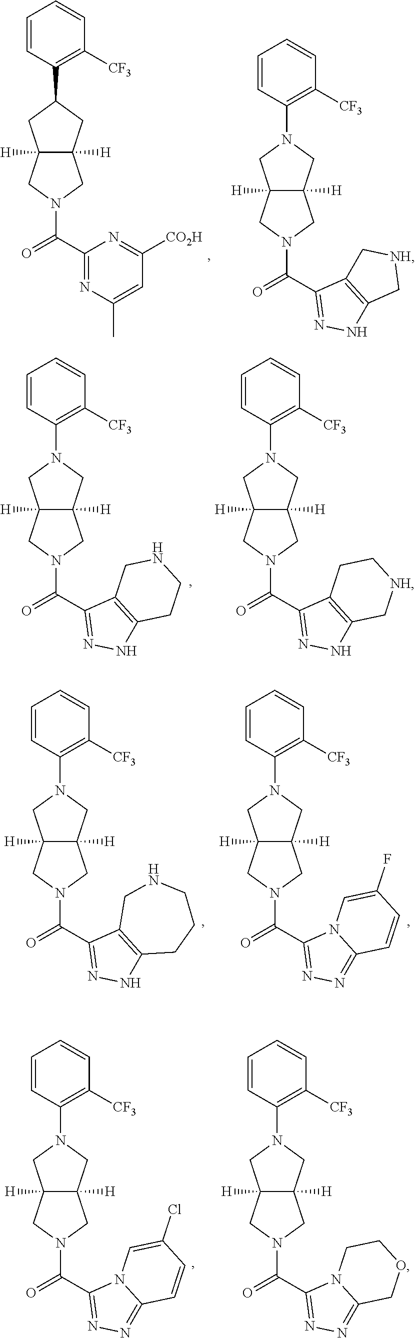 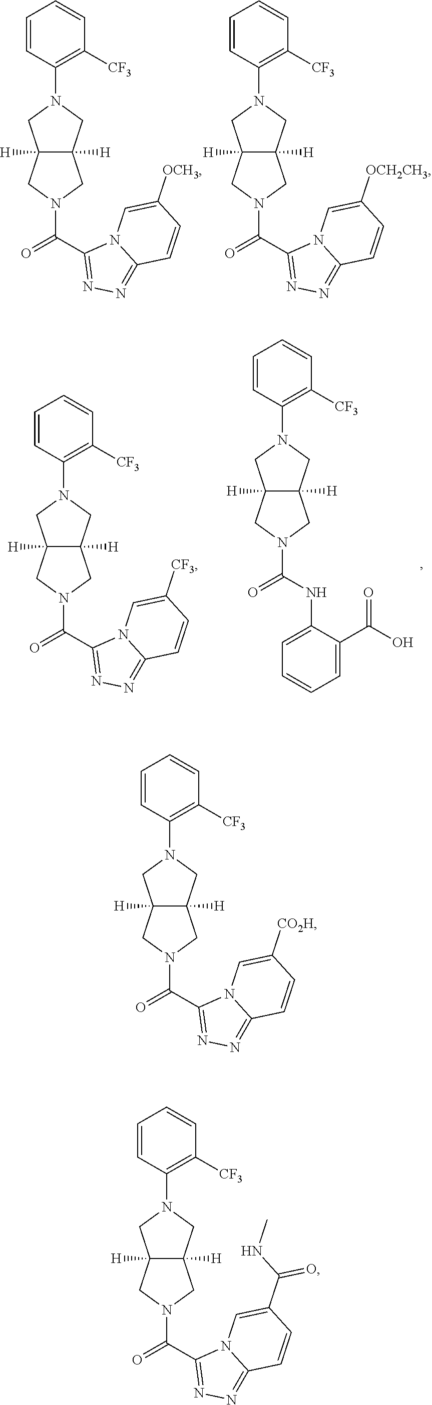 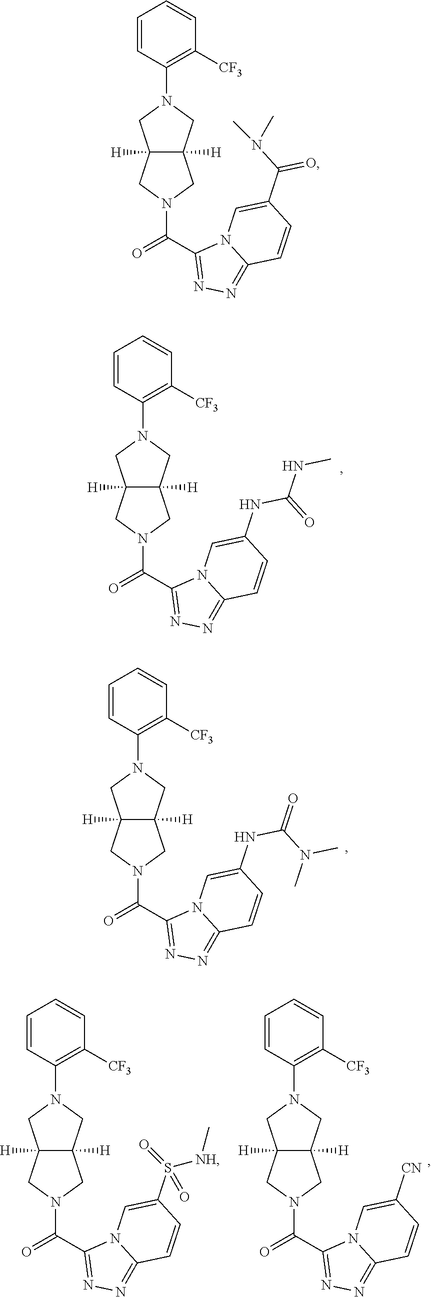 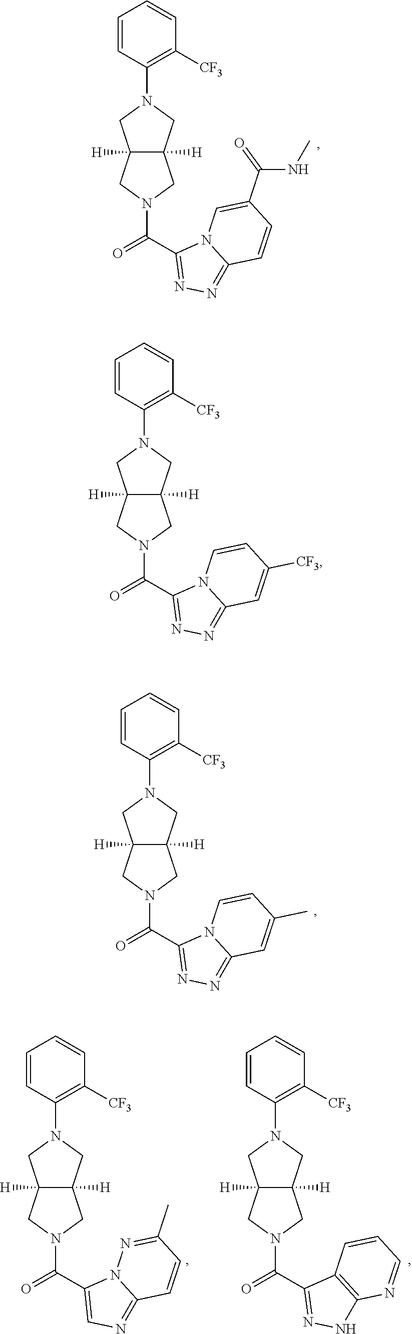 [SMILES: CNC(=O)c1ccc2nnc(C(=O)N3C[C@@H]4CN(c5ccccc5C(F)(F)F)C[C@@H]4C3)n2c1 ; O=C(c1nnc2cc(C(F)(F)F)ccn12)N1C[C@@H]2CN(c3ccccc3C(F)(F)F)C[C@@H]2C1 ; Cc1ccn2c(C(=O)N3C[C@@H]4CN(c5ccccc5C(F)(F)F)C[C@@H]4C3)nnc2c1 ; Cc1ccc2ncc(C(=O)N3C[C@@H]4CN(c5ccccc5C(F)(F)F)C[C@@H]4C3)n2n1 ; O=C(c1n[nH]c2ncccc12)N1C[C@@H]2CN(c3ccccc3C(F)(F)F)C[C@@H]2C1] 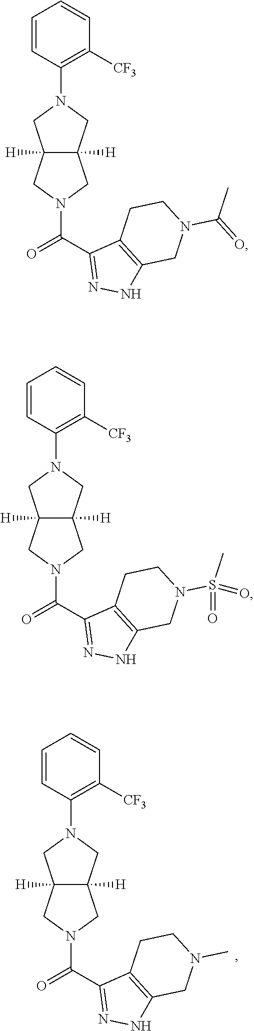 [SMILES: CC(=O)N1CCc2c(C(=O)N3C[C@@H]4CN(c5ccccc5C(F)(F)F)C[C@@H]4C3)n[nH]c2C1 ; CS(=O)(=O)N1CCc2c(C(=O)N3C[C@@H]4CN(c5ccccc5C(F)(F)F)C[C@@H]4C3)n[nH]c2C1 ; CN1CCc2c(C(=O)N3C[C@@H]4CN(c5ccccc5C(F)(F)F)C[C@@H]4C3)n[nH]c2C1] 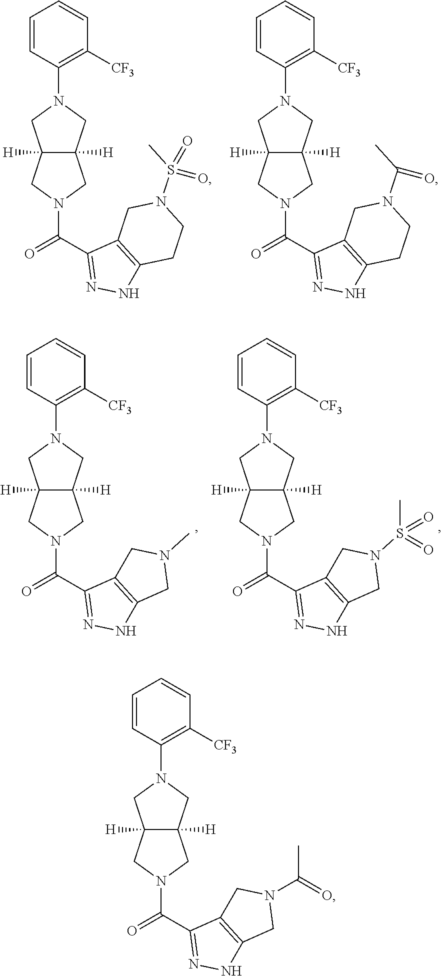 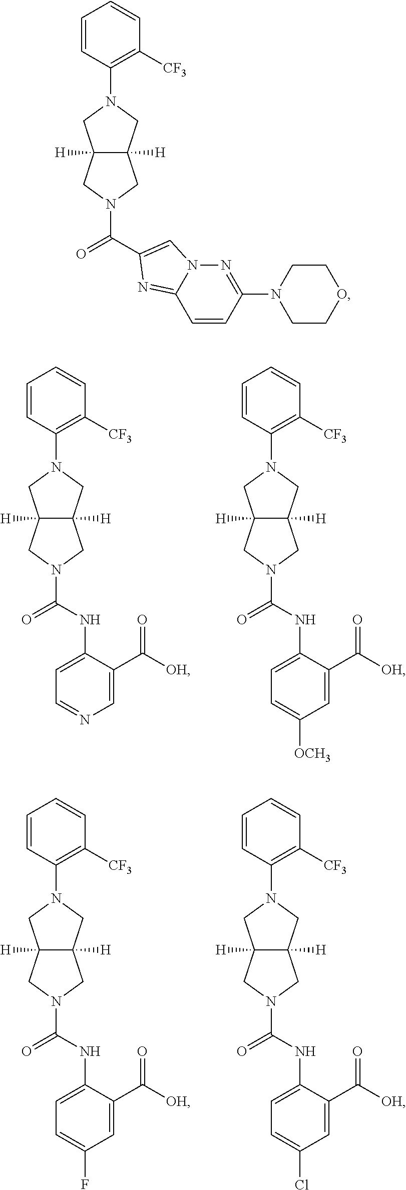 [SMILES: O=C(c1cn2nc(N3CCOCC3)ccc2n1)N1C[C@@H]2CN(c3ccccc3C(F)(F)F)C[C@@H]2C1 ; O=C(O)c1cnccc1NC(=O)N1C[C@@H]2CN(c3ccccc3C(F)(F)F)C[C@@H]2C1 ; COc1ccc(NC(=O)N2C[C@@H]3CN(c4ccccc4C(F)(F)F)C[C@@H]3C2)c(C(=O)O)c1 ; O=C(O)c1cc(F)ccc1NC(=O)N1C[C@@H]2CN(c3ccccc3C(F)(F)F)C[C@@H]2C1 ; O=C(O)c1cc(Cl)ccc1NC(=O)N1C[C@@H]2CN(c3ccccc3C(F)(F)F)C[C@@H]2C1] 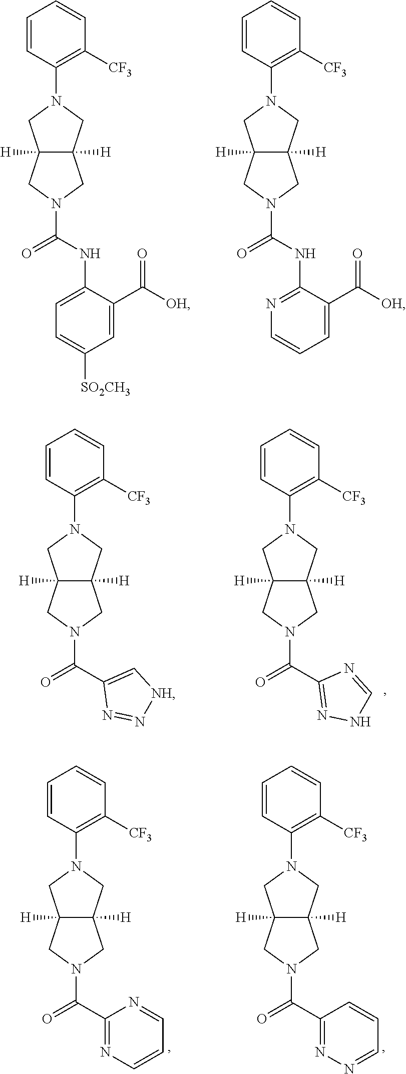 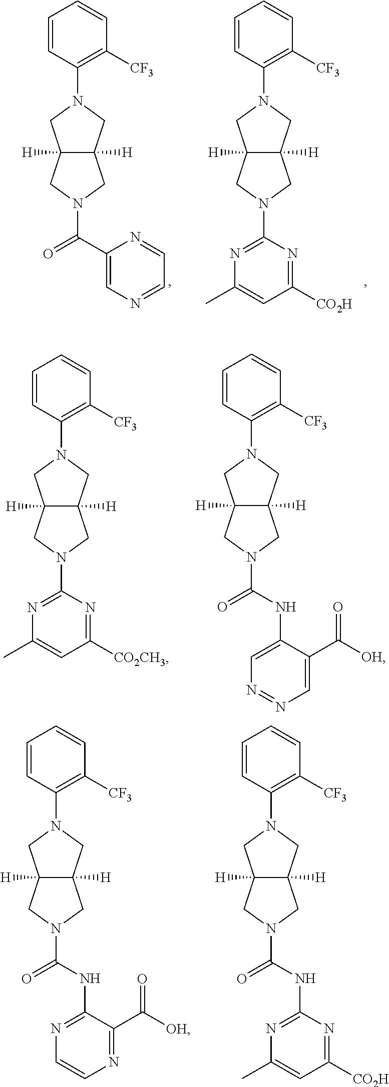 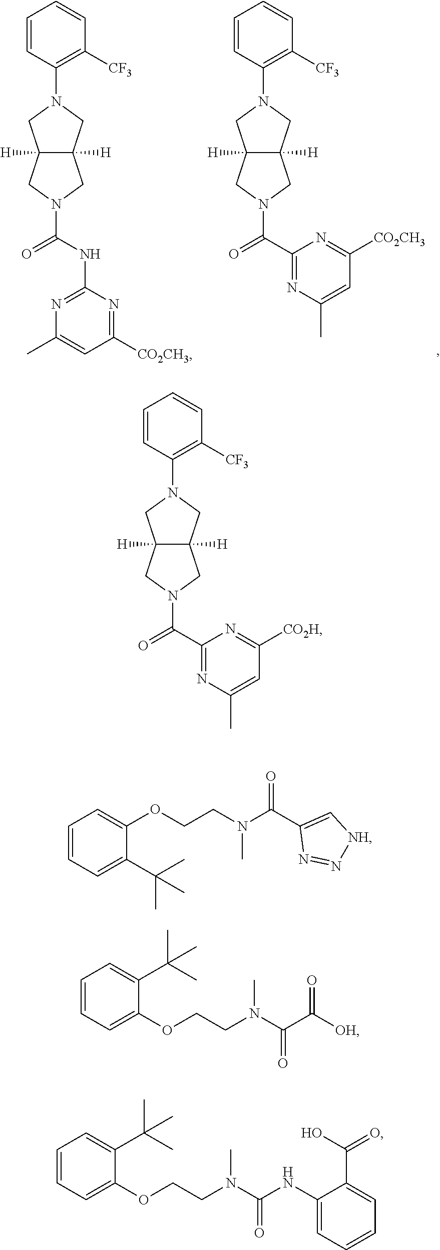 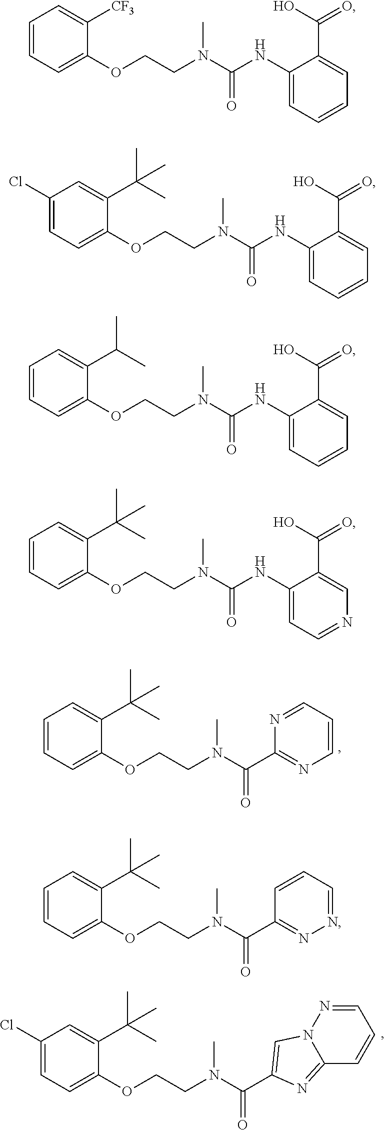 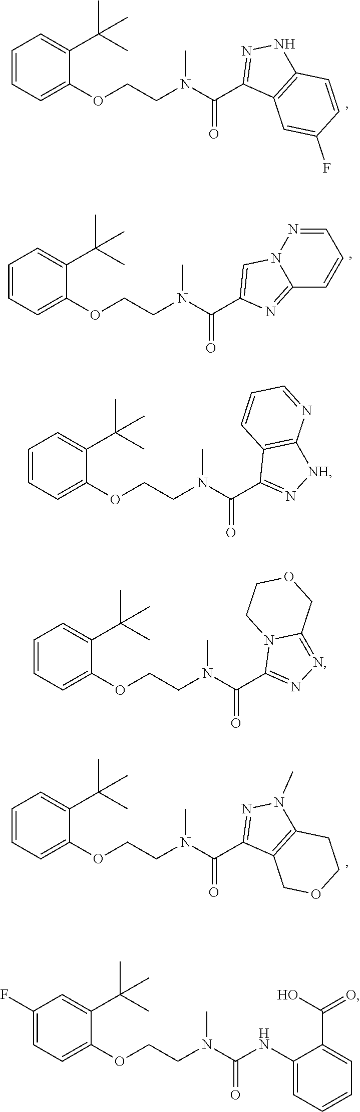 [SMILES: CN(CCOc1ccccc1C(C)(C)C)C(=O)c1n[nH]c2ccc(F)cc12 ; CN(CCOc1ccccc1C(C)(C)C)C(=O)c1cn2ncccc2n1 ; CN(CCOc1ccccc1C(C)(C)C)C(=O)c1n[nH]c2ncccc12 ; CN(CCOc1ccccc1C(C)(C)C)C(=O)c1nnc2n1CCOC2 ; CN(CCOc1ccccc1C(C)(C)C)C(=O)c1nn(C)c2c1COCC2 ; CN(CCOc1ccc(F)cc1C(C)(C)C)C(=O)Nc1ccccc1C(=O)O] 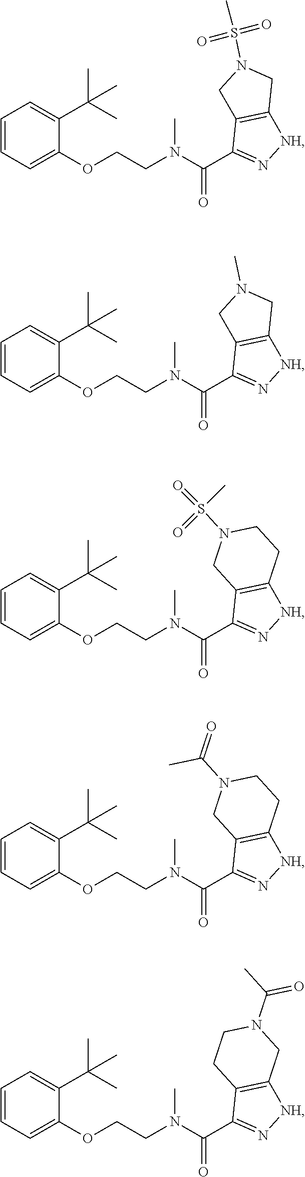 [SMILES: CN(CCOc1ccccc1C(C)(C)C)C(=O)c1n[nH]c2c1CN(S(C)(=O)=O)C2 ; CN1Cc2[nH]nc(C(=O)N(C)CCOc3ccccc3C(C)(C)C)c2C1 ; CN(CCOc1ccccc1C(C)(C)C)C(=O)c1n[nH]c2c1CN(S(C)(=O)=O)CC2 ; CC(=O)N1CCc2[nH]nc(C(=O)N(C)CCOc3ccccc3C(C)(C)C)c2C1 ; CC(=O)N1CCc2c(C(=O)N(C)CCOc3ccccc3C(C)(C)C)n[nH]c2C1] 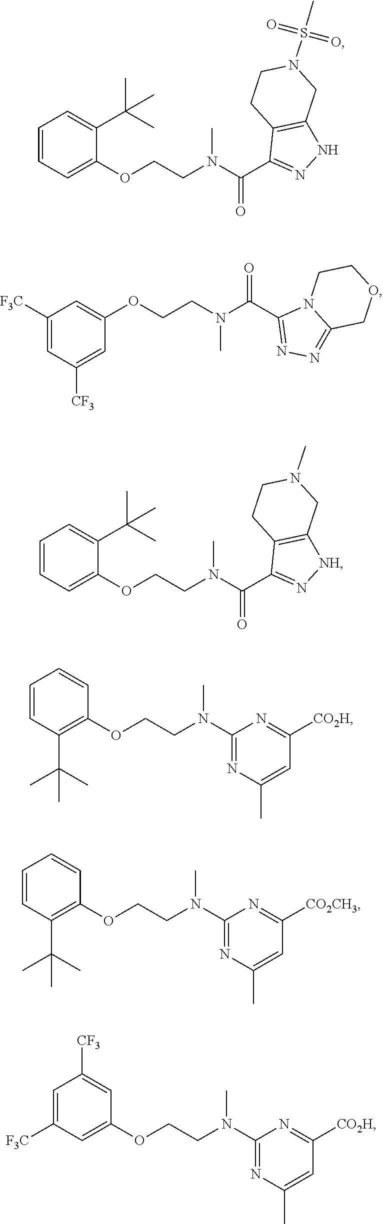 [SMILES: CN(CCOc1ccccc1C(C)(C)C)C(=O)c1n[nH]c2c1CCN(S(C)(=O)=O)C2 ; CN(CCOc1cc(C(F)(F)F)cc(C(F)(F)F)c1)C(=O)c1nnc2n1CCOC2 ; CN1CCc2c(C(=O)N(C)CCOc3ccccc3C(C)(C)C)n[nH]c2C1 ; Cc1cc(C(=O)O)nc(N(C)CCOc2ccccc2C(C)(C)C)n1 ; COC(=O)c1cc(C)nc(N(C)CCOc2ccccc2C(C)(C)C)n1 ; Cc1cc(C(=O)O)nc(N(C)CCOc2cc(C(F)(F)F)cc(C(F)(F)F)c2)n1] 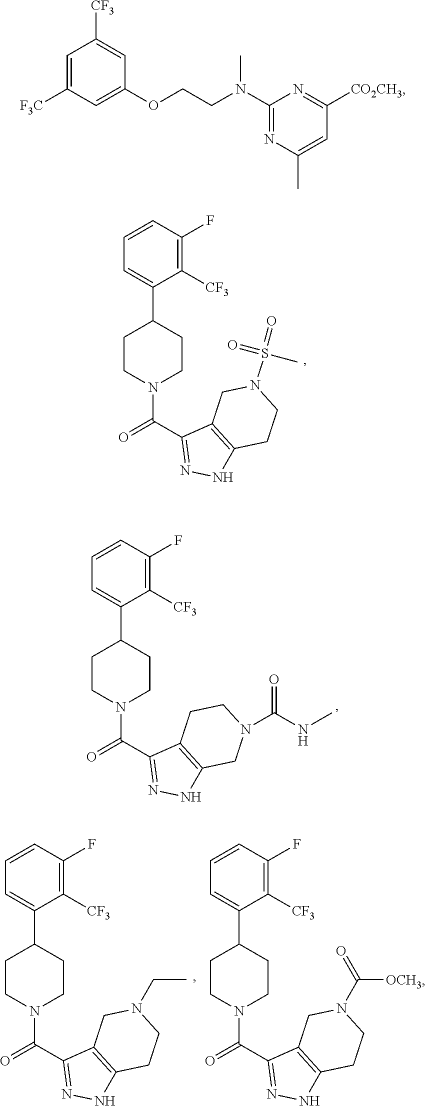 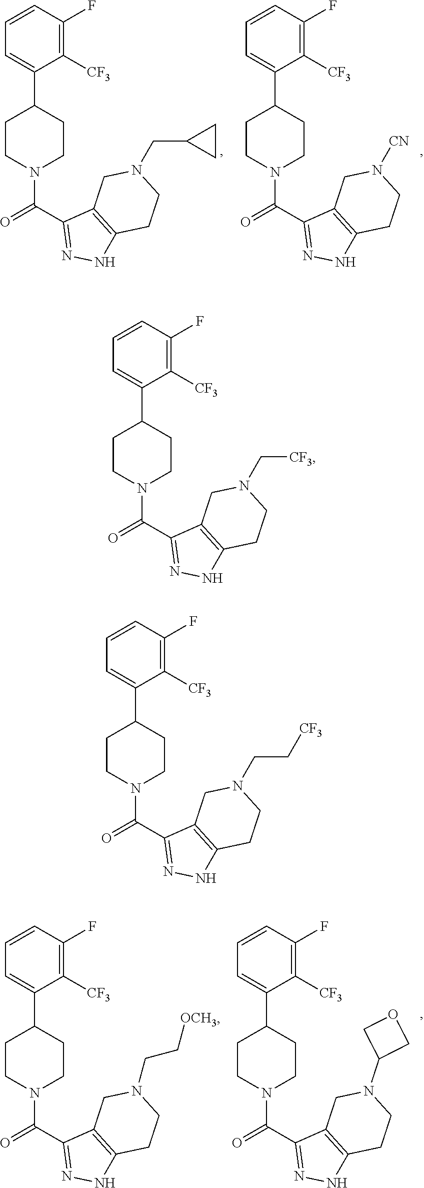 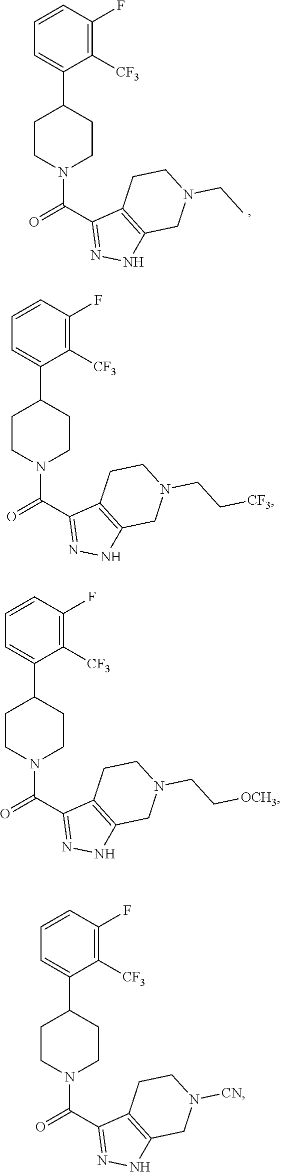 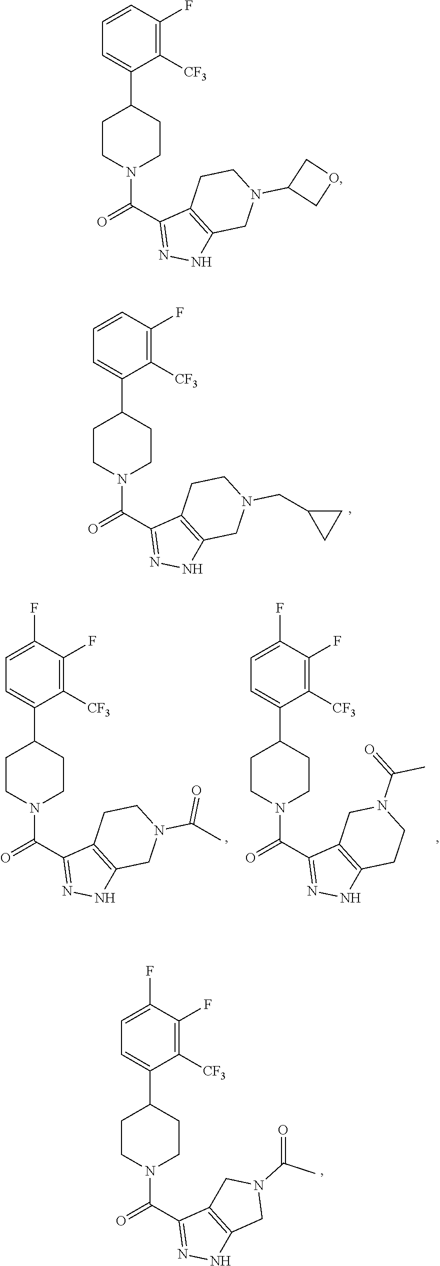 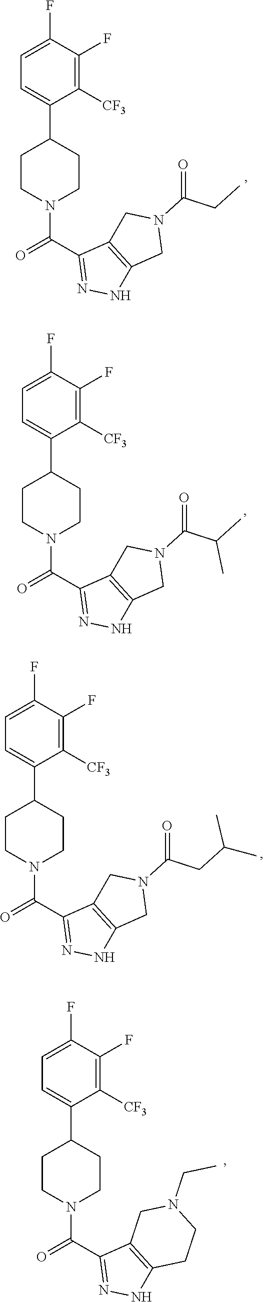 [SMILES: CCC(=O)N1Cc2[nH]nc(C(=O)N3CCC(c4ccc(F)c(F)c4C(F)(F)F)CC3)c2C1 ; CC(C)C(=O)N1Cc2[nH]nc(C(=O)N3CCC(c4ccc(F)c(F)c4C(F)(F)F)CC3)c2C1 ; CC(C)CC(=O)N1Cc2[nH]nc(C(=O)N3CCC(c4ccc(F)c(F)c4C(F)(F)F)CC3)c2C1 ; CCN1CCc2[nH]nc(C(=O)N3CCC(c4ccc(F)c(F)c4C(F)(F)F)CC3)c2C1] 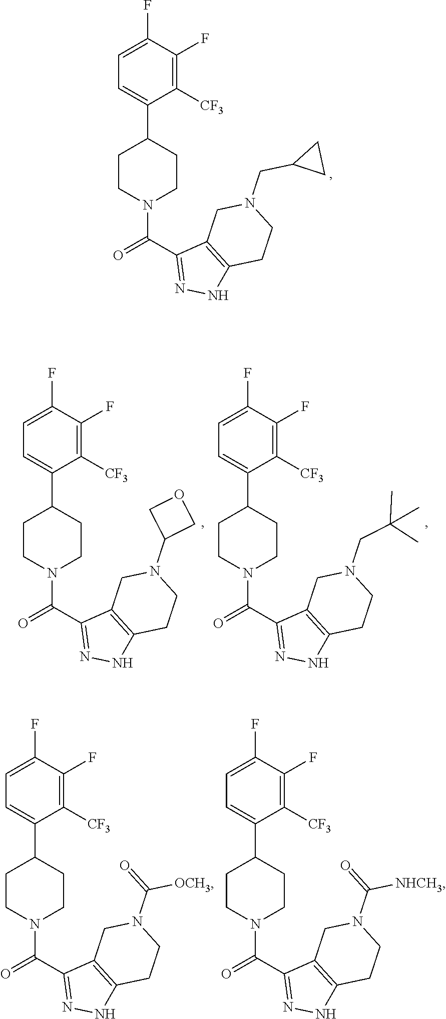 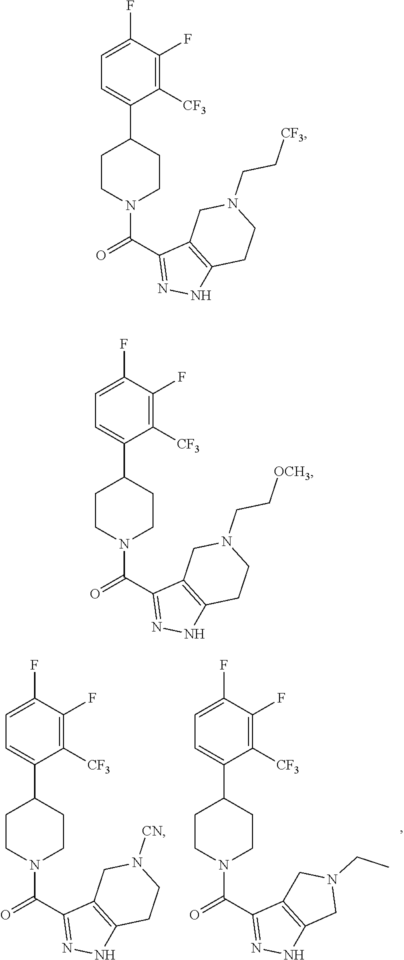 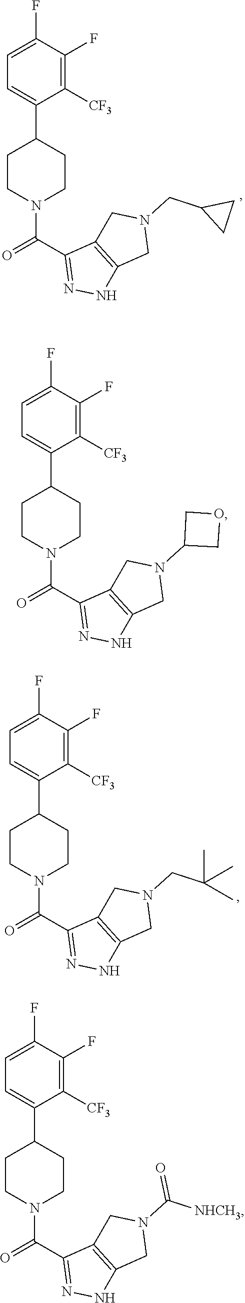 [SMILES: O=C(c1n[nH]c2c1CN(CC1CC1)C2)N1CCC(c2ccc(F)c(F)c2C(F)(F)F)CC1 ; O=C(c1n[nH]c2c1CN(C1COC1)C2)N1CCC(c2ccc(F)c(F)c2C(F)(F)F)CC1 ; CC(C)(C)CN1Cc2[nH]nc(C(=O)N3CCC(c4ccc(F)c(F)c4C(F)(F)F)CC3)c2C1 ; CNC(=O)N1Cc2[nH]nc(C(=O)N3CCC(c4ccc(F)c(F)c4C(F)(F)F)CC3)c2C1] 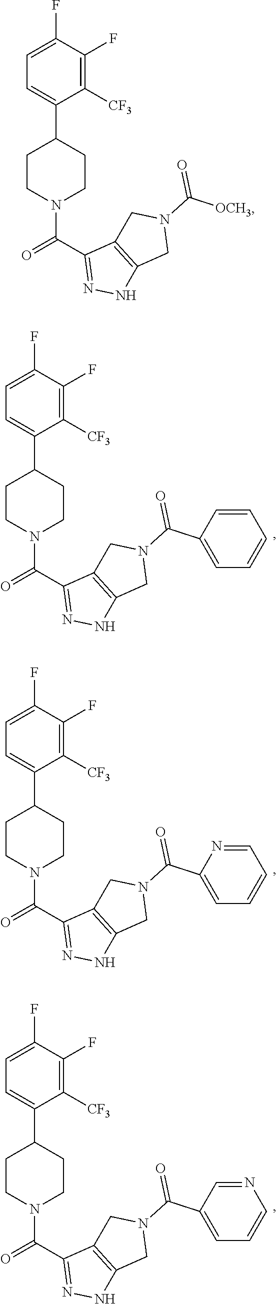 [SMILES: COC(=O)N1Cc2[nH]nc(C(=O)N3CCC(c4ccc(F)c(F)c4C(F)(F)F)CC3)c2C1 ; O=C(c1ccccc1)N1Cc2[nH]nc(C(=O)N3CCC(c4ccc(F)c(F)c4C(F)(F)F)CC3)c2C1 ; O=C(c1ccccn1)N1Cc2[nH]nc(C(=O)N3CCC(c4ccc(F)c(F)c4C(F)(F)F)CC3)c2C1 ; O=C(c1cccnc1)N1Cc2[nH]nc(C(=O)N3CCC(c4ccc(F)c(F)c4C(F)(F)F)CC3)c2C1] 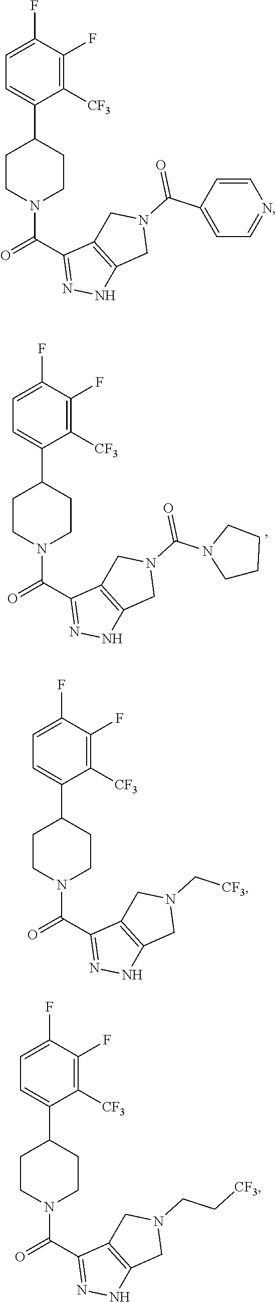 [SMILES: O=C(c1ccncc1)N1Cc2[nH]nc(C(=O)N3CCC(c4ccc(F)c(F)c4C(F)(F)F)CC3)c2C1 ; O=C(c1n[nH]c2c1CN(C(=O)N1CCCC1)C2)N1CCC(c2ccc(F)c(F)c2C(F)(F)F)CC1 ; O=C(c1n[nH]c2c1CN(CC(F)(F)F)C2)N1CCC(c2ccc(F)c(F)c2C(F)(F)F)CC1 ; O=C(c1n[nH]c2c1CN(CCC(F)(F)F)C2)N1CCC(c2ccc(F)c(F)c2C(F)(F)F)CC1] 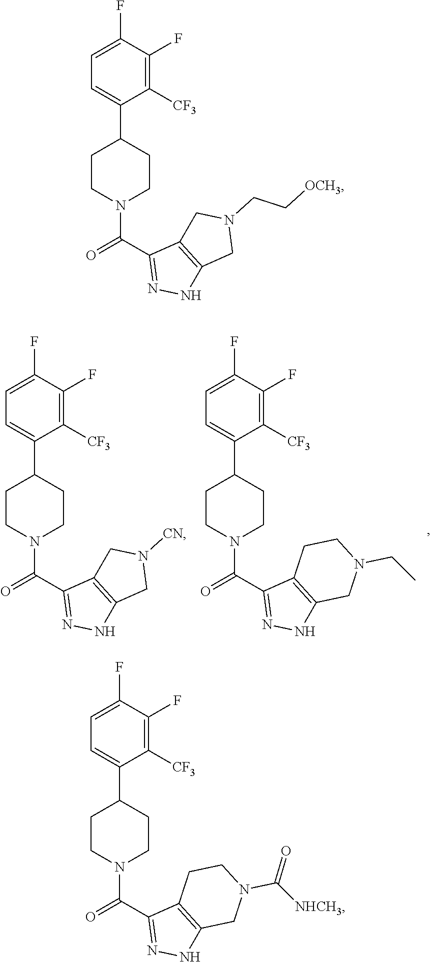 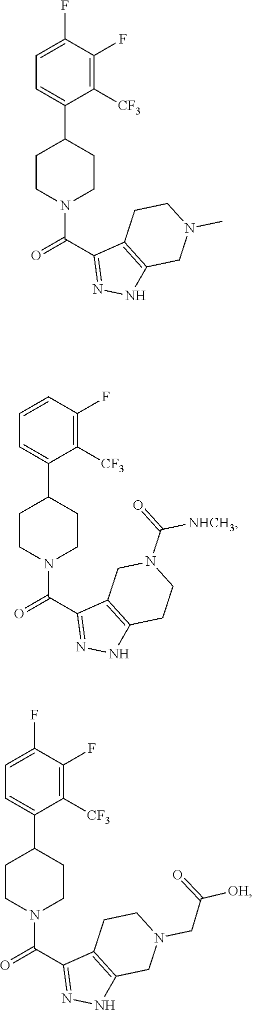 [SMILES: CN1CCc2c(C(=O)N3CCC(c4ccc(F)c(F)c4C(F)(F)F)CC3)n[nH]c2C1 ; CNC(=O)N1CCc2[nH]nc(C(=O)N3CCC(c4cccc(F)c4C(F)(F)F)CC3)c2C1 ; O=C(O)CN1CCc2c(C(=O)N3CCC(c4ccc(F)c(F)c4C(F)(F)F)CC3)n[nH]c2C1] 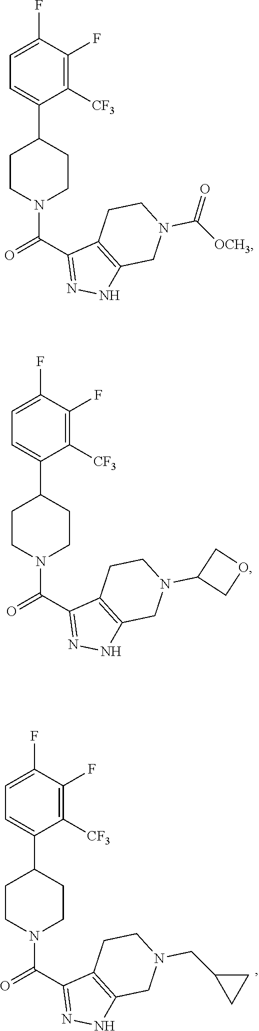 [SMILES: COC(=O)N1CCc2c(C(=O)N3CCC(c4ccc(F)c(F)c4C(F)(F)F)CC3)n[nH]c2C1 ; O=C(c1n[nH]c2c1CCN(C1COC1)C2)N1CCC(c2ccc(F)c(F)c2C(F)(F)F)CC1 ; O=C(c1n[nH]c2c1CCN(CC1CC1)C2)N1CCC(c2ccc(F)c(F)c2C(F)(F)F)CC1] 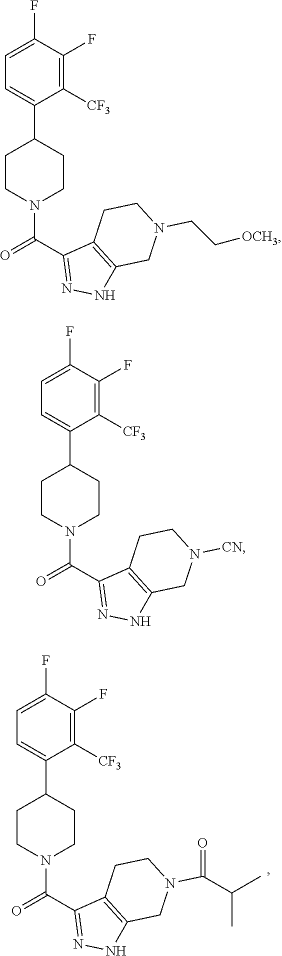 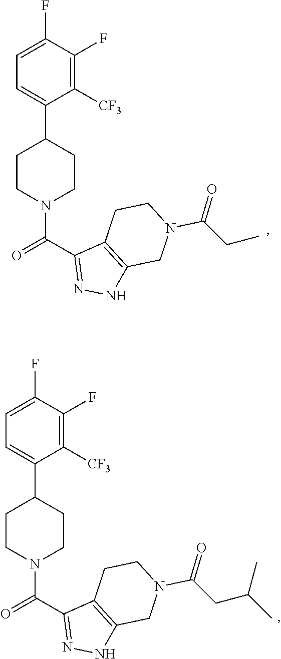 [SMILES: CCC(=O)N1CCc2c(C(=O)N3CCC(c4ccc(F)c(F)c4C(F)(F)F)CC3)n[nH]c2C1 ; CC(C)CC(=O)N1CCc2c(C(=O)N3CCC(c4ccc(F)c(F)c4C(F)(F)F)CC3)n[nH]c2C1] 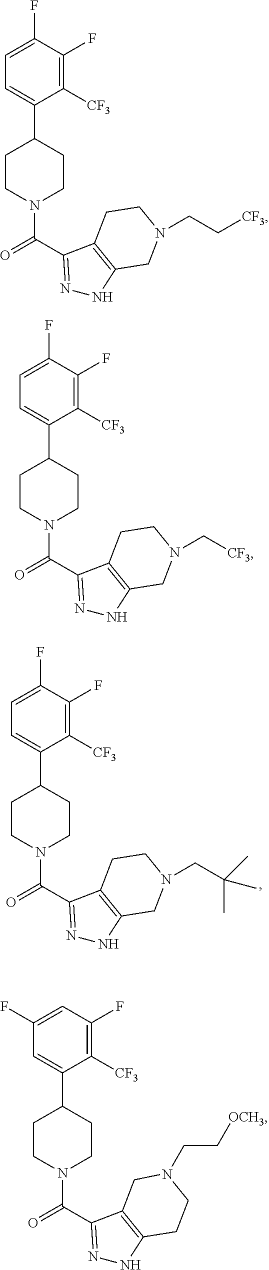 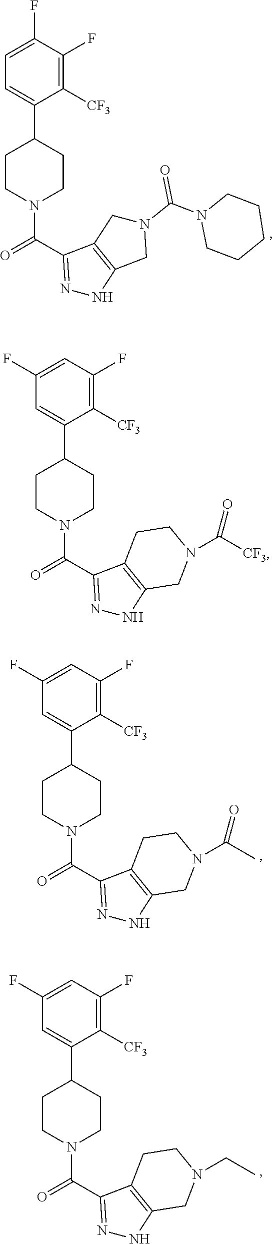 [SMILES: O=C(c1n[nH]c2c1CN(C(=O)N1CCCCC1)C2)N1CCC(c2ccc(F)c(F)c2C(F)(F)F)CC1 ; O=C(c1n[nH]c2c1CCN(C(=O)C(F)(F)F)C2)N1CCC(c2cc(F)cc(F)c2C(F)(F)F)CC1 ; CC(=O)N1CCc2c(C(=O)N3CCC(c4cc(F)cc(F)c4C(F)(F)F)CC3)n[nH]c2C1 ; CCN1CCc2c(C(=O)N3CCC(c4cc(F)cc(F)c4C(F)(F)F)CC3)n[nH]c2C1] 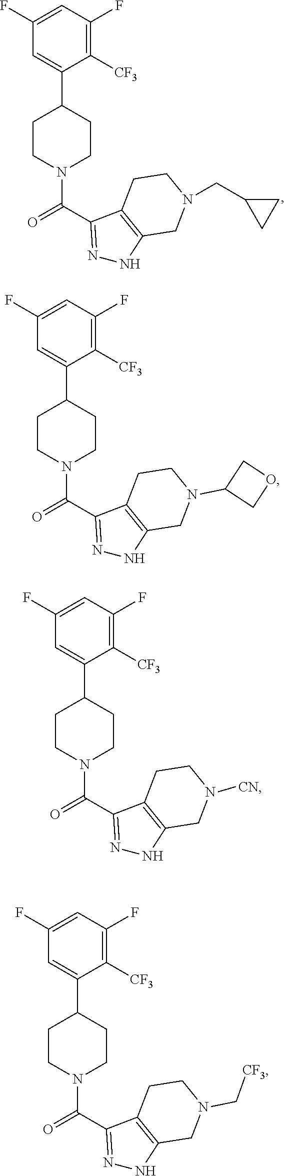 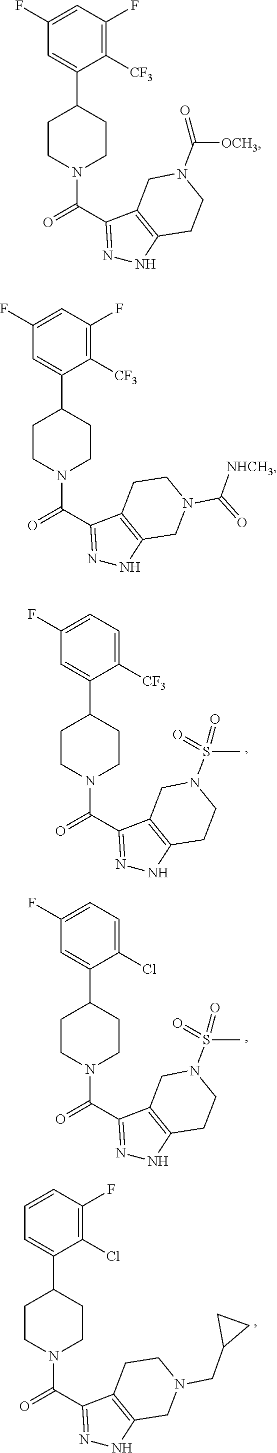 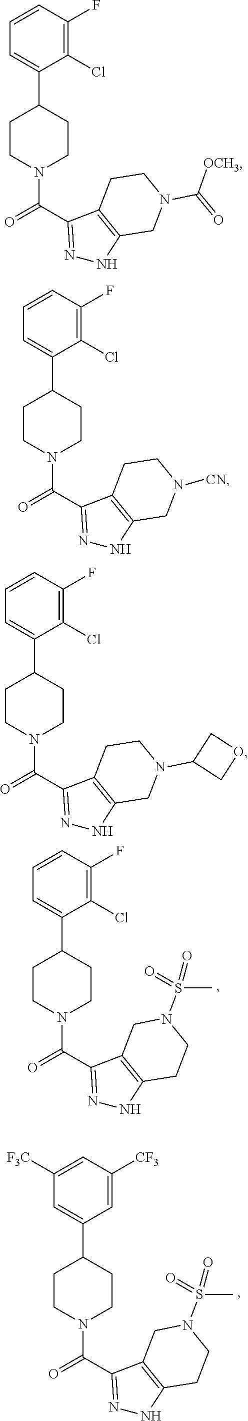 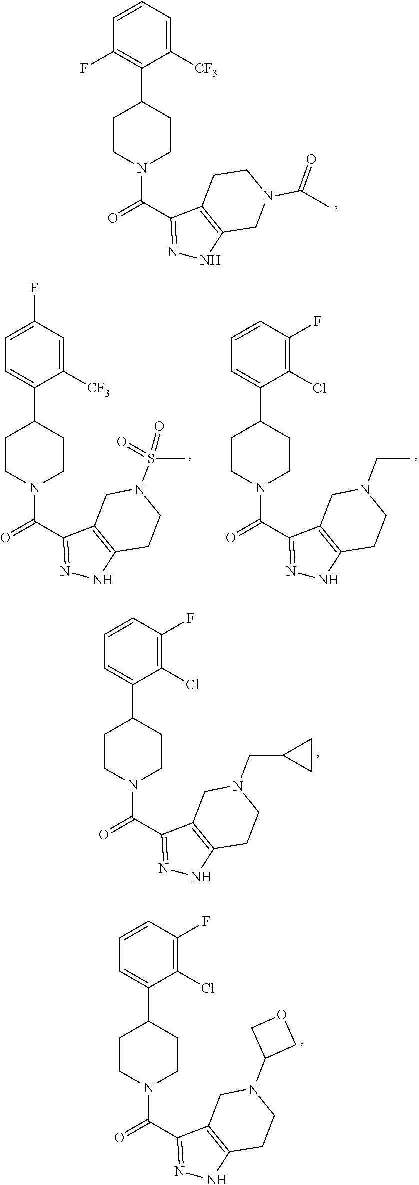 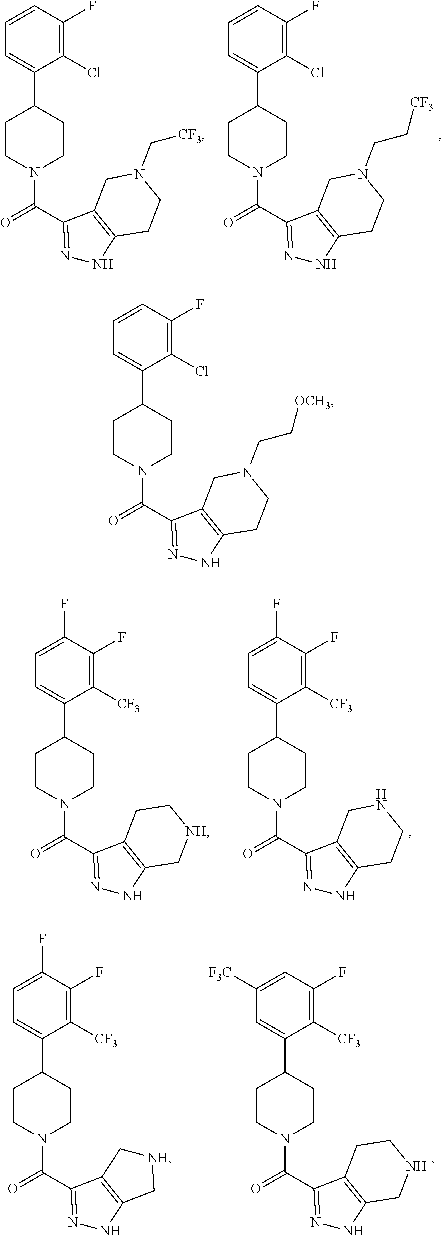 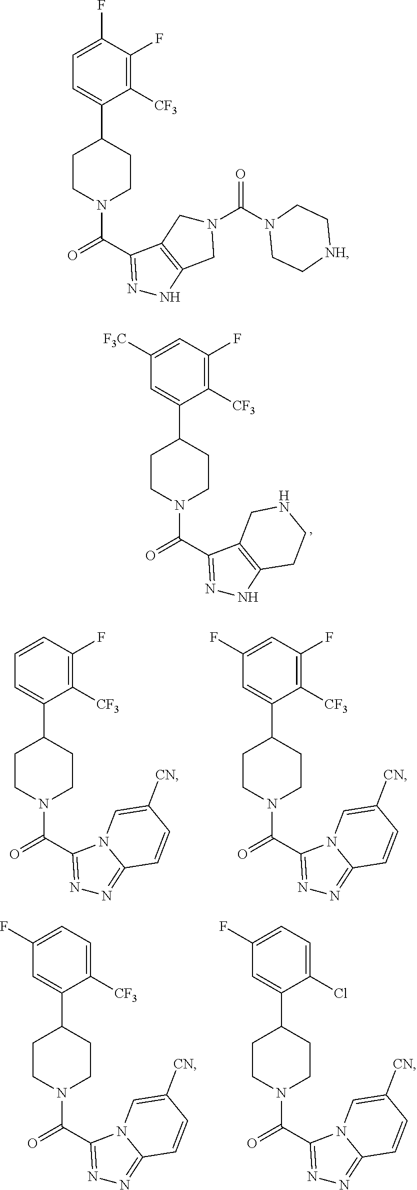 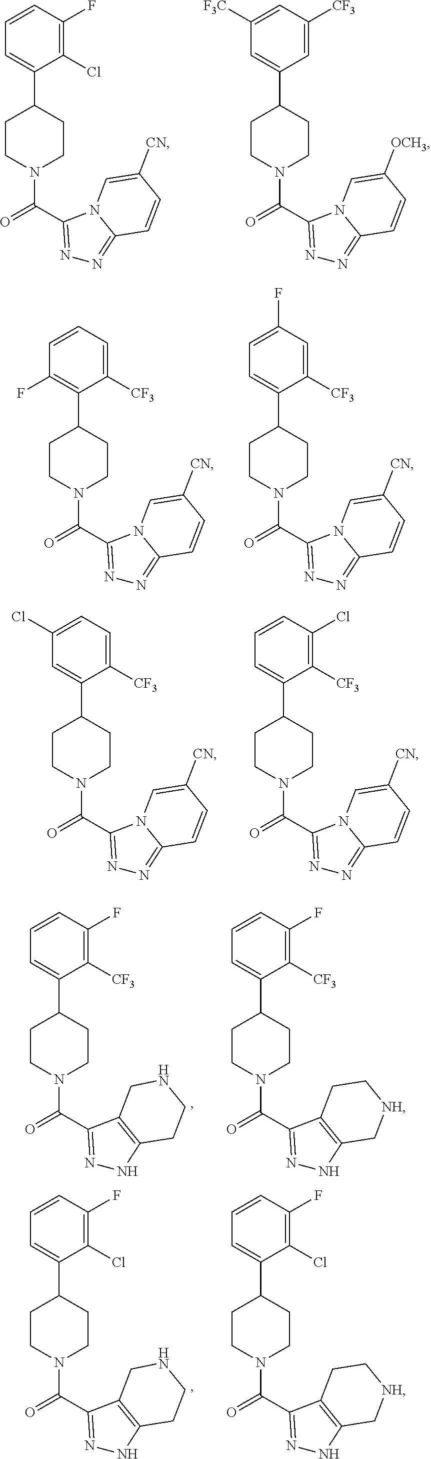 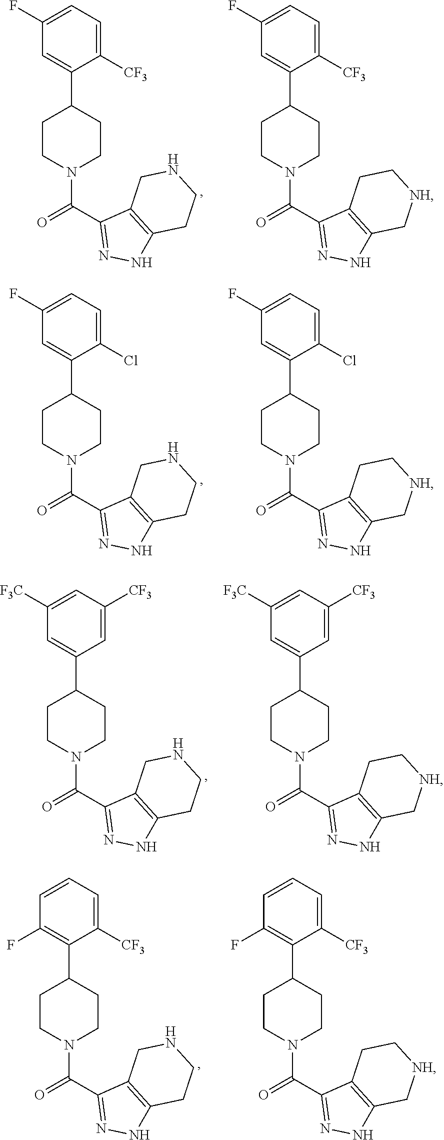 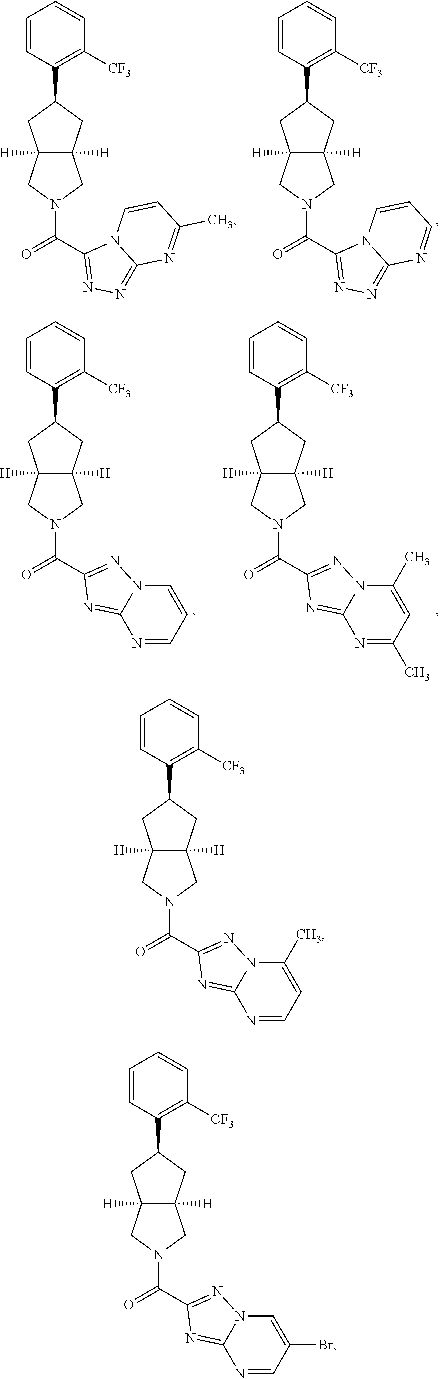 [SMILES: Cc1ccn2c(C(=O)N3C[C@H]4C[C@@H](c5ccccc5C(F)(F)F)C[C@H]4C3)nnc2n1 ; O=C(c1nnc2ncccn12)N1C[C@H]2C[C@@H](c3ccccc3C(F)(F)F)C[C@H]2C1 ; O=C(c1nc2ncccn2n1)N1C[C@H]2C[C@@H](c3ccccc3C(F)(F)F)C[C@H]2C1 ; Cc1cc(C)n2nc(C(=O)N3C[C@H]4C[C@@H](c5ccccc5C(F)(F)F)C[C@H]4C3)nc2n1 ; Cc1ccnc2nc(C(=O)N3C[C@H]4C[C@@H](c5ccccc5C(F)(F)F)C[C@H]4C3)nn12 ; O=C(c1nc2ncc(Br)cn2n1)N1C[C@H]2C[C@@H](c3ccccc3C(F)(F)F)C[C@H]2C1] 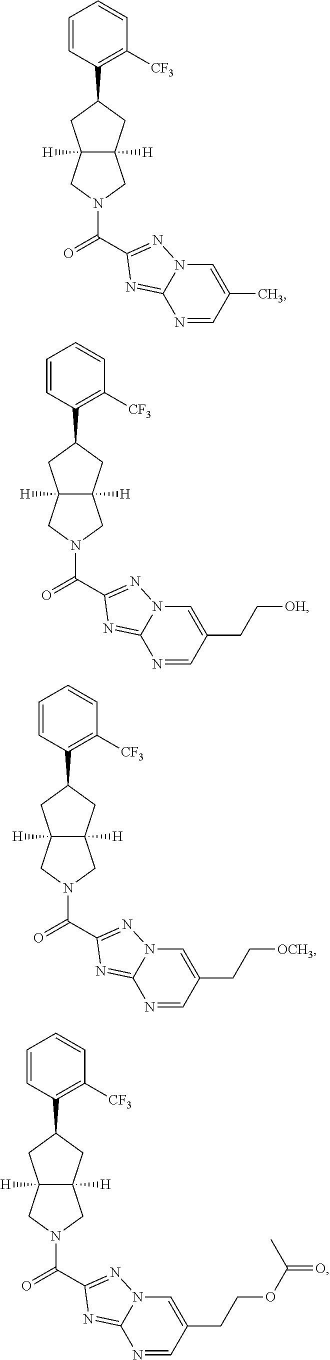 [SMILES: Cc1cnc2nc(C(=O)N3C[C@H]4C[C@@H](c5ccccc5C(F)(F)F)C[C@H]4C3)nn2c1 ; O=C(c1nc2ncc(CCO)cn2n1)N1C[C@H]2C[C@@H](c3ccccc3C(F)(F)F)C[C@H]2C1 ; COCCc1cnc2nc(C(=O)N3C[C@H]4C[C@@H](c5ccccc5C(F)(F)F)C[C@H]4C3)nn2c1 ; CC(=O)OCCc1cnc2nc(C(=O)N3C[C@H]4C[C@@H](c5ccccc5C(F)(F)F)C[C@H]4C3)nn2c1] 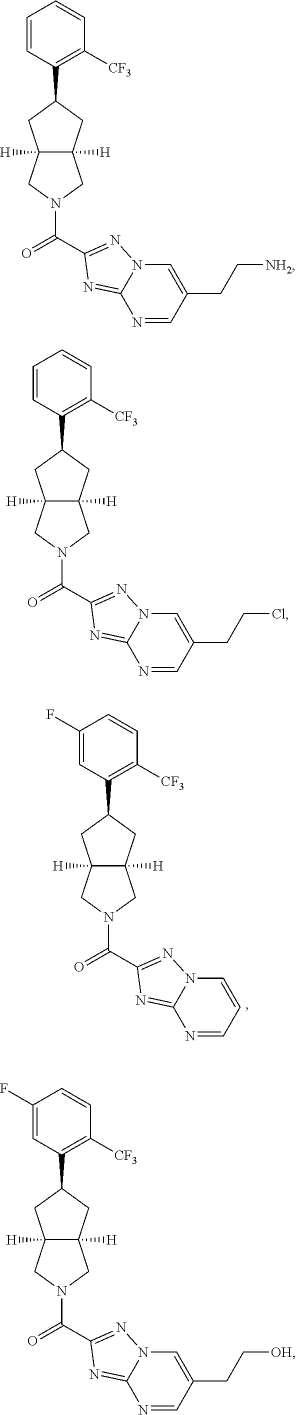 [SMILES: NCCc1cnc2nc(C(=O)N3C[C@H]4C[C@@H](c5ccccc5C(F)(F)F)C[C@H]4C3)nn2c1 ; O=C(c1nc2ncc(CCCl)cn2n1)N1C[C@H]2C[C@@H](c3ccccc3C(F)(F)F)C[C@H]2C1 ; O=C(c1nc2ncccn2n1)N1C[C@H]2C[C@@H](c3cc(F)ccc3C(F)(F)F)C[C@H]2C1 ; O=C(c1nc2ncc(CCO)cn2n1)N1C[C@H]2C[C@@H](c3cc(F)ccc3C(F)(F)F)C[C@H]2C1] 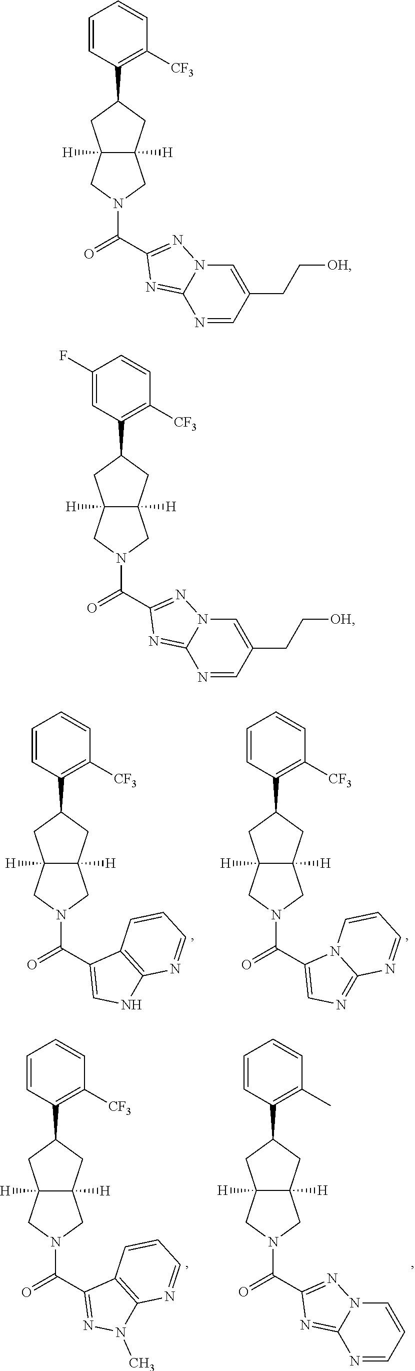 [SMILES: O=C(c1nc2ncc(CCO)cn2n1)N1C[C@H]2C[C@@H](c3ccccc3C(F)(F)F)C[C@H]2C1 ; O=C(c1nc2ncc(CCO)cn2n1)N1C[C@H]2C[C@@H](c3cc(F)ccc3C(F)(F)F)C[C@H]2C1 ; O=C(c1c[nH]c2ncccc12)N1C[C@H]2C[C@@H](c3ccccc3C(F)(F)F)C[C@H]2C1 ; O=C(c1cnc2ncccn12)N1C[C@H]2C[C@@H](c3ccccc3C(F)(F)F)C[C@H]2C1 ; Cn1nc(C(=O)N2C[C@H]3C[C@@H](c4ccccc4C(F)(F)F)C[C@H]3C2)c2cccnc21 ; Cc1ccccc1[C@@H]1C[C@@H]2CN(C(=O)c3nc4ncccn4n3)C[C@@H]2C1] 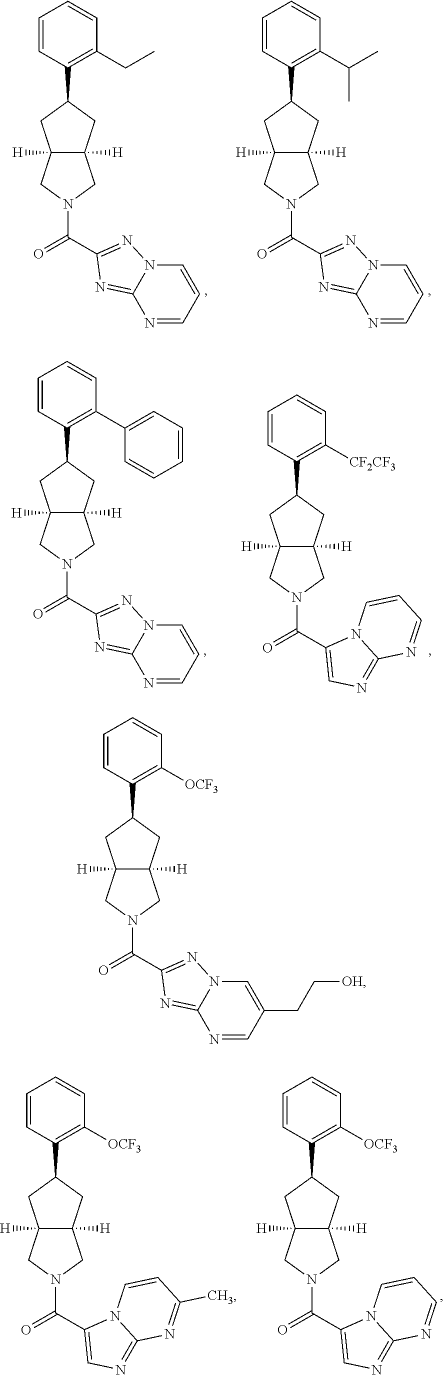 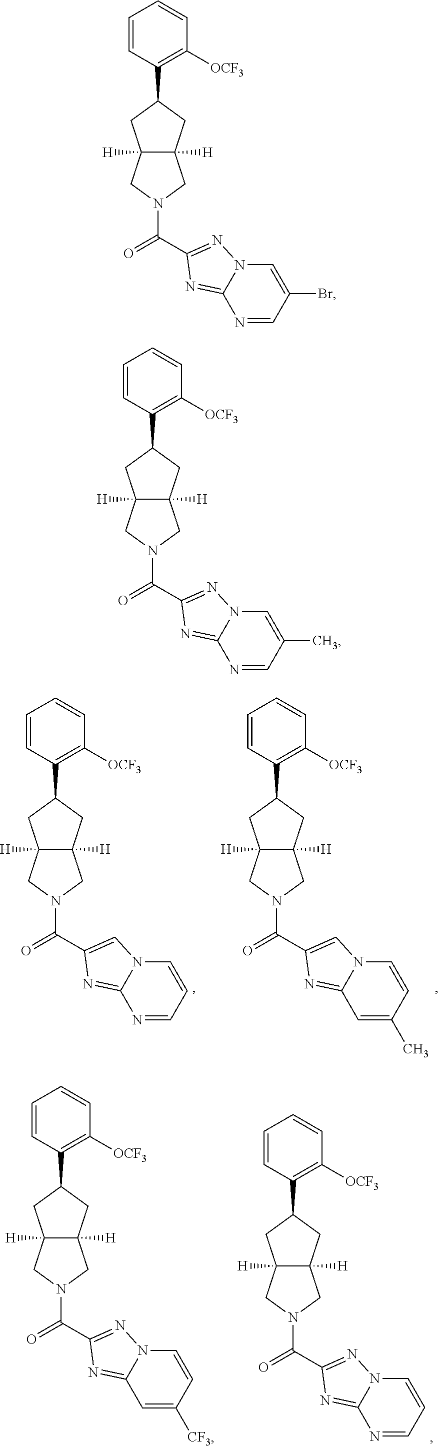 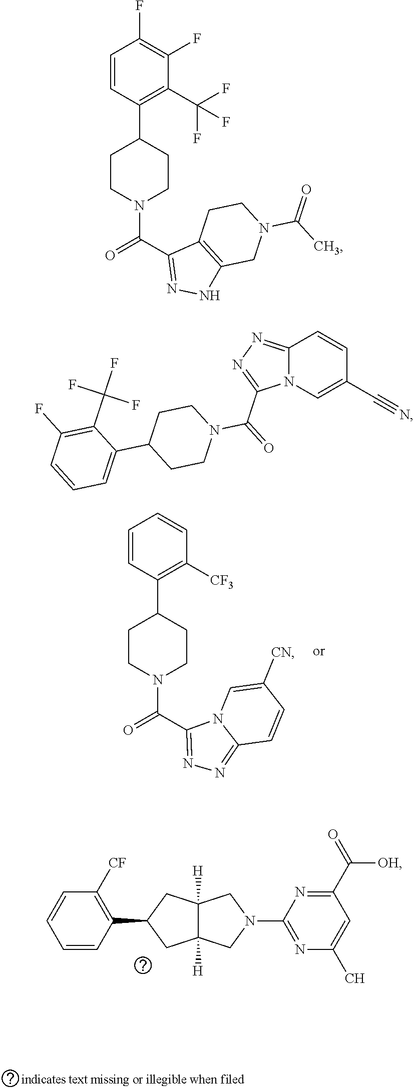
or a pharmaceutically acceptable salt thereof.

9-15. (canceled)

16. The compound of claim 1, wherein:
a is 2 and b is 1; or
a is 1 and b is 1; or
a is 2, b is 1, and M is 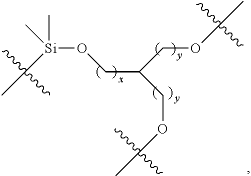
wherein x is 1, 2, 3, 4, or 5, and y is 1, 2, 3, 4, or 5;
or
a is 2, b is 1, and M is 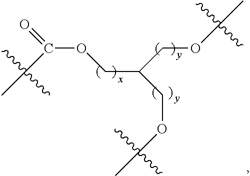
wherein x is 1, 2, 3, 4, or 5, and y is 1, 2, 3, 4, or 5;
or
a is 2, b is 1, and M is 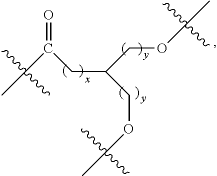
wherein x is 0, 1, 2, 3, or 4, and y is 1, 2, 3, 4, or 5;
or
a is 1, b is 1, and M is 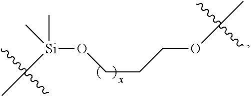
wherein x is 1, 2, 3, 4, or 5;
or
a is 1, b is 1, and M is 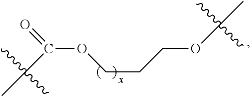
wherein x is 1, 2, 3, 4, or 5;
or
a is 1, b is 1, and M is 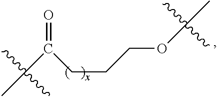
wherein x is 0, 1, 2, 3, or 4, and/or
wherein P is 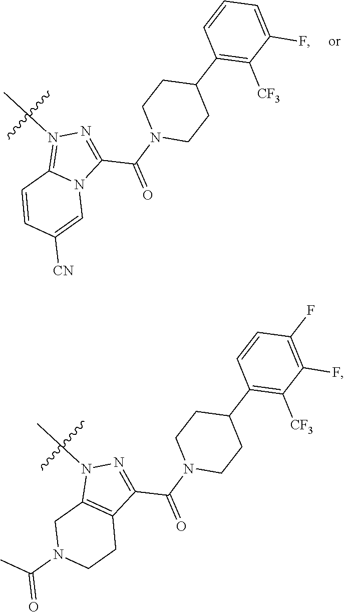
and/or
wherein Q is 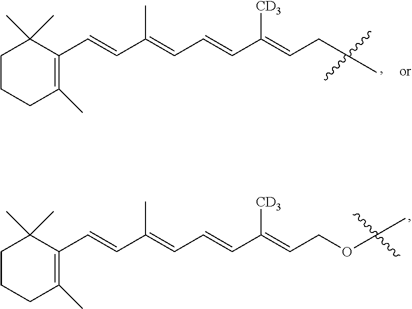 [SMILES: *C/C=C(/C=C/C=C(C)/C=C/C1=C(C)CCCC1(C)C)C([2H])([2H])[2H] ; *OC/C=C(/C=C/C=C(C)/C=C/C1=C(C)CCCC1(C)C)C([2H])([2H])[2H]]
and/or
wherein M is 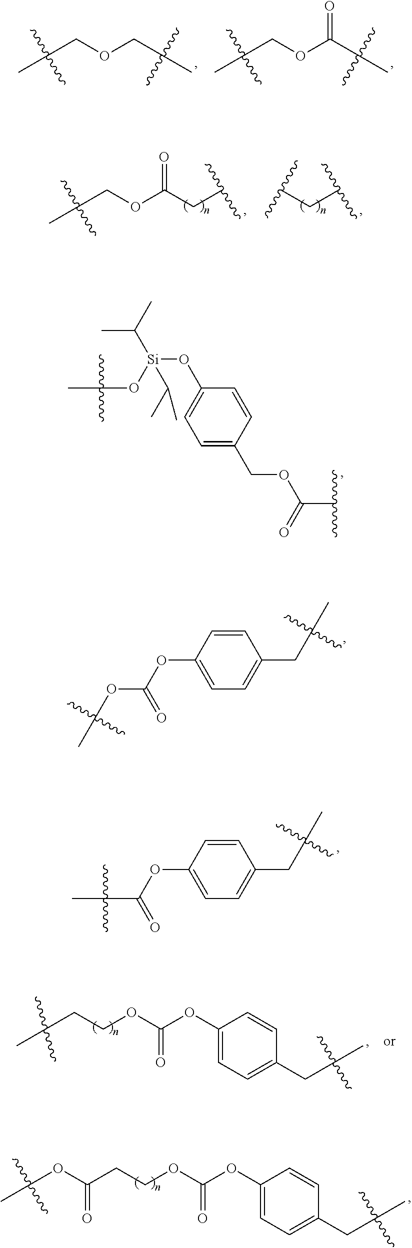
wherein n is 0-20.

17-19. (canceled)

20. The compound of claim 1 having the following structure: 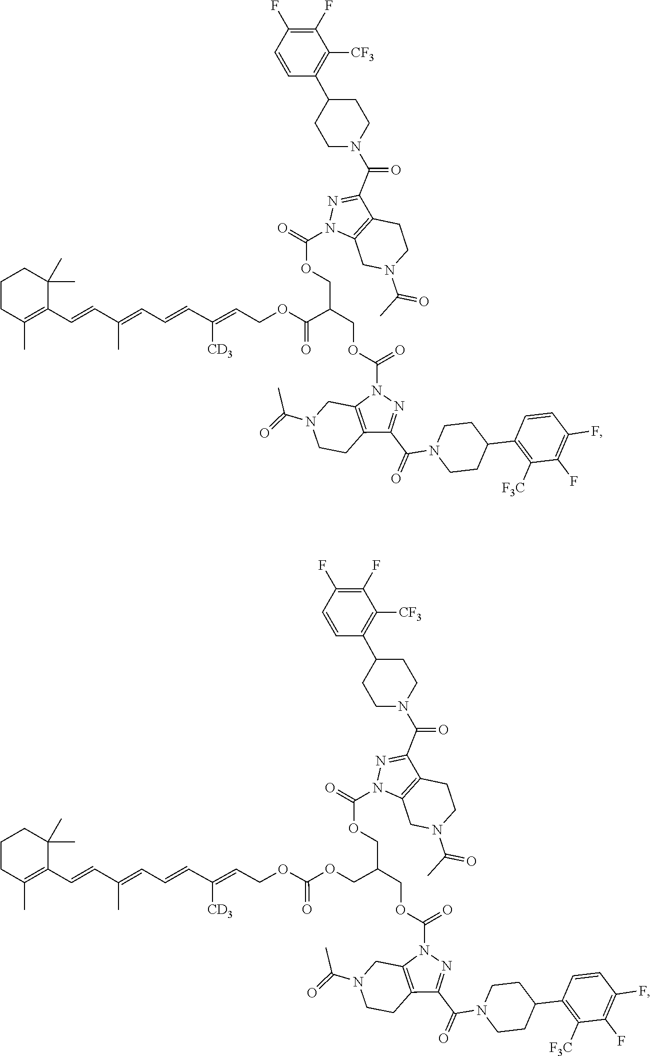 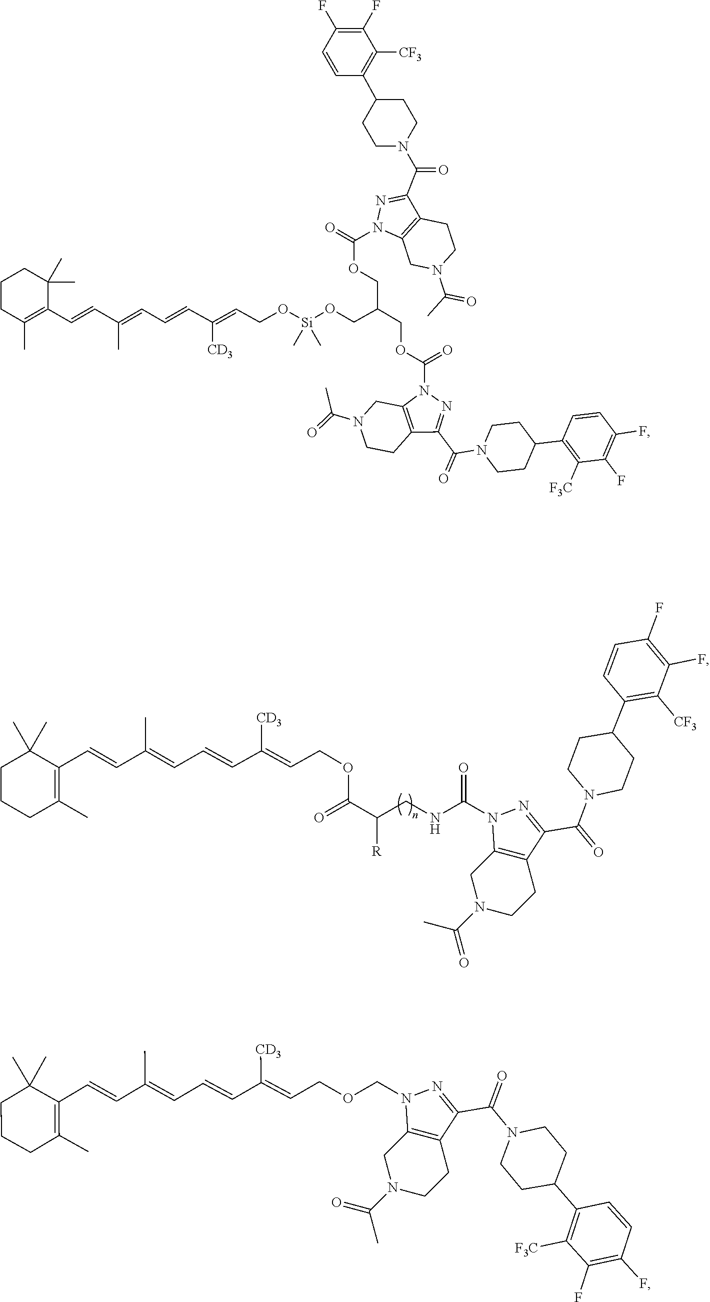 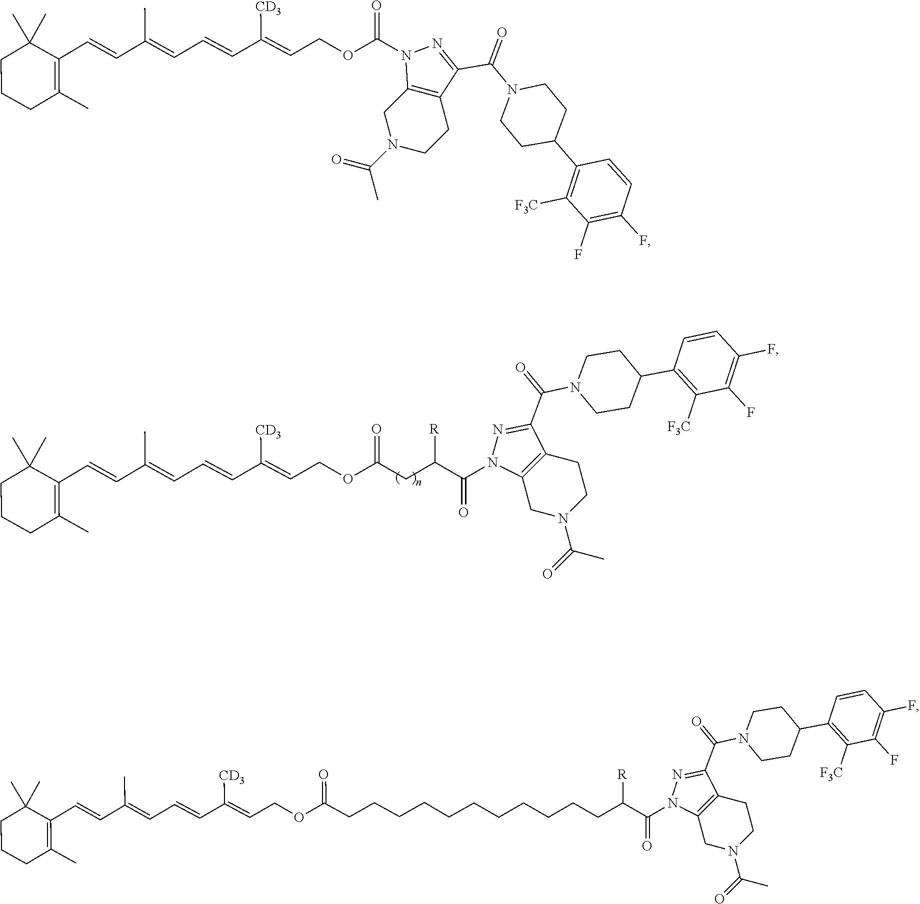
wherein n is 0-20; R is H, Trp, Phe, Leu, or Tyr, 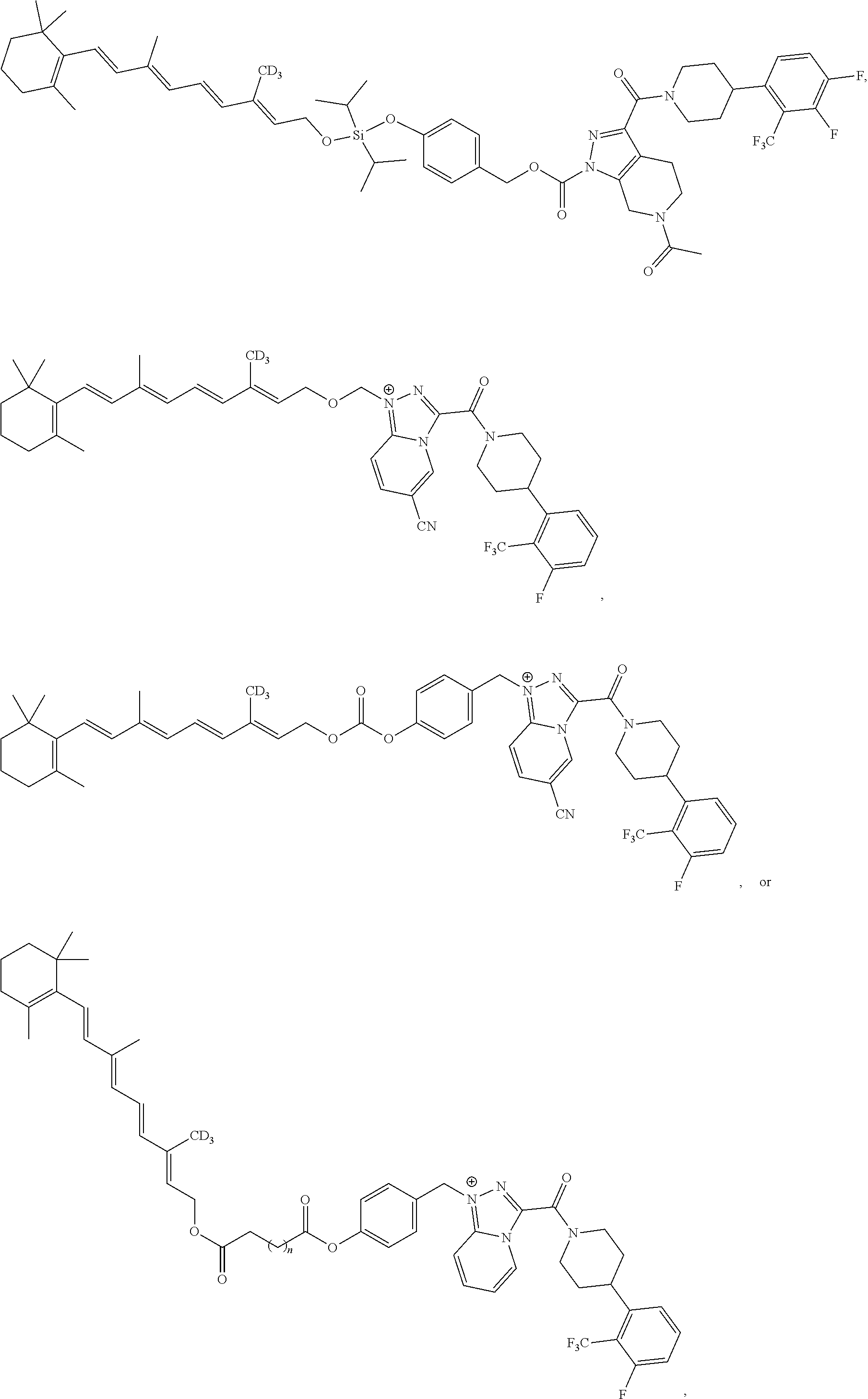
wherein n is 0-20.

21. A pharmaceutical composition comprising the compound of claim 1 and a pharmaceutically acceptable carrier or a pharmaceutical composition comprising the selective RBP4 antagonist from claim 1 and a C20-D3-visual-chromophore-producing compound, preferably, the C20-D3-visual-chromophore-producing compound is selected from the group consisting of C20-D3-retinol, C20-D3-retinaldehyde, C20-D3-retinyl esters, C20-D3-9-cis-retinol, C20-D3-9-cis-retinaldehye, C20-D3-9-cis-retinyl esters, C20-D3-11-cis-retinol, C20-D3-11-cis-retinaldehye, C20-D3-11-cis-retinyl esters, and C20-D3-C20′-D3-β-carotene; more preferably the C20-D3-visual-chromophore-producing compound is selected from the group consisting of C20-D3-retinol, C20-D3-9-cis-retinol, and C20-D3-11-cis-retinol.

22. A method for treating a disease characterized by excessive or age-related lipofuscin accumulation in the retina in a mammal afflicted therewith, and for promoting rhodopsin and cone opsins production, comprising administering to the mammal an effective amount of the compound of the pharmaceutical composition of claim 21; preferably comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first,
preferably, wherein the disease is further characterized by bisretinoid-mediated macular degeneration, or
wherein the disease characterized by age-related lipofuscin accumulation in the retina is
(a) Age-Related Macular Degeneration;
(b) dry (atrophic) Age-Related Macular Degeneration;
(c) Stargardt Disease or other forms of retinopathy caused by or associated with mutations in the ABCA4 gene, such as retinitis pigmentosa (RP19) or cone-rod dystrophy (CORD3);
(d) Best disease;
(e) adult vitelliform maculopathy; or
(f) Stargardt-like macular dystrophy.

23. (canceled)

24. The method of claim 22, wherein the amount of the compound or the amount of the pharmaceutical composition is effective to lower the serum concentration of RBP4 in the mammal, or wherein the amount of the compound or the amount of the pharmaceutical composition is effective to lower the retinal concentration of a bisretinoid in lipofuscin in the mammal,
preferably, wherein the bisretinoid is A2E, isoA2E, A2-DHP-PE, or at RAL di-PE.

25-26. (canceled)

27. The method of claim 22, wherein the administration is effective to reduce photoreceptor degeneration.

28. A method for
(a) treating type 2 diabetes and for promoting rhodopsin and cone opsins production in a mammal, comprising administering to the mammal an amount of the pharmaceutical composition of claim 21 effective to treat type 2 diabetes and to promote rhodopsin and cone opsins production, preferably comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first,
(b) treating obesity and for promoting rhodopsin and cone opsins production in a mammal, comprising administering to the mammal an amount of the pharmaceutical composition of claim 21 effective to treat obesity and to promote rhodopsin and cone opsins production, preferably comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first,
(c) treating insulin resistance and for promoting rhodopsin and cone opsins production in a mammal, comprising administering to the mammal an amount of the pharmaceutical composition of claim 21 effective to treat insulin resistance and to promote rhodopsin and cone opsins production, preferably comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first,
(d) treating cardiovascular disease and for promoting rhodopsin and cone opsins production in a mammal, comprising administering to the mammal an amount of the pharmaceutical composition of claim 21 effective to treat cardiovascular disease and to promote rhodopsin and cone opsins production, preferably comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first,
(e) treating hepatic steatosis and for promoting rhodopsin and cone opsins production in a mammal, comprising administering to the mammal an amount of the pharmaceutical composition of claim 21 effective to treat hepatic steatosis and to promote rhodopsin and cone opsins production, preferably comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first, or
(f) treating a non-alcoholic fatty liver disease (NAFLD) in a mammal afflicted therewith, and for promoting rhodopsin and cone opsins production, comprising administering to the mammal an amount of the pharmaceutical composition of claim 21 effective to treat a non-alcoholic fatty liver disease (NAFLD) and to promote rhodopsin and cone opsins production, optionally comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first.

29-32. (canceled)

33. A method for
(a) treating a non-alcoholic fatty liver disease (NAFLD) in a mammal afflicted therewith, and for promoting rhodopsin and cone opsins production, comprising administering to the mammal an amount of the pharmaceutical composition of claim 21 effective to treat a non-alcoholic fatty liver disease (NAFLD) and to promote rhodopsin and cone opsins production, optionally comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first,
preferably, wherein the mammal is afflicted with a NAFLD selected from the group consisting of hepatic steatosis (fatty liver), non-alcoholic steatohepatitis (NASH), cirrhosis, and hepatocellular carcinoma, or
(b) treating gout in a mammal afflicted therewith, and for promoting rhodopsin and cone opsins production, comprising administering to the mammal an amount of the pharmaceutical composition of claim 21 effective to treat gout and to promote rhodopsin and cone opsins production, preferably comprising a sequential, simultaneous, contemporaneous, or concomitant administration of the compound or composition and the C20-D3-visual-chromophore-producing compound; preferably, in a sequential administration, the selective RBP4 antagonist may be administered first or the C20-D3-visual-chromophore-producing compound may be administered first,
preferably wherein the mammal is afflicted with gout;
preferably, the gout is chronic gout or acute gout.

34-35. (canceled)

36. The method of claim 33, wherein the method further comprises
(a) a step of determining, or having determined, the level of RBP4 in adipose tissue in the mammal and administering to the mammal the compound or the pharmaceutical composition if the level of RBP4 in adipose tissue is elevated; or
(b) a step of determining, or having determined, the level of RBP4 in serum in the mammal and administering to the mammal the compound or the pharmaceutical composition if the level of RBP4 in serum is elevated.

37. The method of any ene of claim 33, wherein the amount of the compound or the amount of the pharmaceutical composition is
(a) effective in reducing RBP4 levels in adipose tissue in the mammal or in reducing RBP4 levels in serum in the mammal;
(b) effective in reducing uric acid levels in the serum of the mammal;
(c) effective to normalize the concentration of triglycerides in the liver of the mammal, is effective to normalize the concentration of free fatty acids in the serum of the mammal, or is effective to normalize the concentration of free fatty acids in the liver of the mammal; or
(d) effective to prevent trafficking of a fatty acid by RBP4, is effective to prevent trafficking of a fatty acid to the liver by RBP4, or is effective to inhibit binding between RBP4 and a fatty acid-,
preferably, wherein
(i) the fatty acid is from adipose tissue;
(ii) the mammal has elevated serum RBP4 levels; or
(iii) the serum RBP4 level of the mammal is elevated by more than 3 micrograms per ml.

38. (canceled)

39. The method of claim 33, wherein the amount of the compound administered is such that the amount of the selective RBP4 antagonist released in the body of the mammal is 5-1000 mg, 5-800 mg, 5-200 mg, 45-200 mg, 45-1000 mg, 45-800 mg, 10-50 mg, 96 mg, 24 mg, or 10 mg per day.

40. The method of claim 33, wherein the method further comprises administering an amount of a second agent which is (R)-(+)-(5,6-dichloro 2,3,9,9a-tetrahydro 3-oxo-9a-propyl-1H-fluoren-7-yl)oxy]acetic acid (DPOFA), a Nonsteroidal Anti-inflammatory Drug (NSAID) such as indomethacin, colchicine, lesinurad, corticosteroids (e.g., betamethasone, prednisone, dexamethasone, cortisone, hydrocortisone, methylprednisone, prednisolone), biologic anti-IL-1alpha/beta agents (e.g., canakinumab, rilonacept, anakinra), allopurinol, benzbromarone, pegloticase, and other forms of uricase enzymes, topiroxostat (FYX-051), ulodesine (BCX4208), KUX-1151, RLBN1001, RDEA3170, arhalofenate (MBX-102), levotofisopam, UR-1102, PF-06743649, BCX4208, SHR4640, Lumiracoxib, Tranilast, Topiroxostat, LC350189, Bucillamine, AC-201, HuZhen Capsules (including Polygonum cuspidatum and Ligustrum lucidum), MPC-004, FYU-981, Sodium Bicarbonate, SEL-212, SEL-037, Apremilast, TMX-67, SSS11, D-0120, febuxostat, or probenecid, or esters or salts thereof, effective to treat the mammal,
preferably, wherein the amount of the second agent and/or the amount of the compound or the pharmaceutical composition is effective in reducing uric acid levels in the blood of the mammal or is effective in decreasing uric acid reabsorption in the kidneys of the mammal.

41-42. (canceled)
